(19) United States
(12) Patent Application Publication
(10) Pub. No.: US 2021/0215697 A1
(43) Pub. Date: Jul. 15, 2021

(54) Serum Biomarkers

(71) Applicant: BerGenBio ASA, Bergen (NO)

(72) Inventors: Robert Holt, Bergen (NO); David Micklem, Bergen (NO)

(73) Assignee: BerGenBio ASA, Bergen (NO)

(21) Appl. No.: 17/054,193

(22) PCT Filed: May 13, 2019
(86) PCT No.: PCT/EP2019/062215

§ 371 Date: Nov. 10, 2020

(30) Foreign Application Priority Data
May 14, 2018 (GB) 1807789.1
Oct. 15, 2018 (GB) 1816764.3
Oct. 19, 2018 (GB) 1817024.1

(51) Int. Cl. G01N 33/574; A61K 31/7105; A61K 31/711; C07K 16/28

(52) U.S. Cl. CPC G01N 33/574; A61K 31/7105; G01N 2800/52; C07K 16/2863; G01N 2496/00; A61K 31/711

(57) ABSTRACT

The present disclosure relates to the field of cancer biomarkers and treatments, and more particularly to methods of predicting susceptibility to cancer treatments, in particular treatments with Axl inhibitors. Also disclosed are products, such as kits, having utility in performing the disclosed methods.

### EARLIER APPLICATIONS

This application claims priority from: United Kingdom application GB1807789.1, filed on 14 May 2018; United Kingdom application GB1817024.1, filed on 19 Oct. 2018; and United Kingdom application GB1816764.3, filed on 15 Oct. 2018. The disclosure of these three priority document is incorporated by reference into the present application for all purposes.

### FIELD OF THE DISCLOSURE

The present disclosure relates to the field of cancer treatment. In particular the present disclosure relates to the field of cancer biomarkers and treatments, and more particularly to methods of predicting susceptibility to cancer treatments, as well as products, such as kits, to perform these methods.

### BACKGROUND

Heterogeneous Patient Populations

Cancers are a large family of diseases characterised by abnormal cell growth and having the potential to invade or spread to other parts of the body. Whilst cancers are categorised into classes which share broad characteristics, some of these classes are particularly heterogeneous. As a result, patients in these heterogeneous populations can display heterogeneity in their drug response—that is, patients in heterogeneous cancer groups can have different responses to the same cancer treatment. By way of example, acute myeloid leukemia (AML) is a heterogeneous cancer in which different subjects can have different responses to the same cancer treatment (De Kouchkovsky et al, 2016).

By identifying biomarkers that reliably distinguish between subjects who respond to or benefit from treatment and those who do not, it should be possible to use these biomarkers to predict clinical outcome for such patients.

Predictive Methods

Predictive methods provide information on the likely outcome of a particular treatment regimen, and have the power to guide the use of tailored therapies. Such methods can provide information regarding, for example, the likelihood of a subject responding to a treatment, how aggressively an individual should be treated within a particular treatment regimen, and/or how aggressively an individual should be treated with conventional therapeutic methods such as radiation/chemotherapy.

Despite advances in the field of predictive biomarkers and methods, there remains a need for sensitive, efficient, and rapid non-invasive methods of predicting patient response to cancer treatments in a number of heterogeneous cancers.

The present disclosure has been devised in light of the above considerations.

### SUMMARY OF THE DISCLOSURE

The present disclosure addresses the need for methods of predicting the susceptibility of a subject having, suspected of having, or diagnosed with cancer to cancer treatments by assessing biomarkers in the subject or in a sample obtained from the subject. In particular, the disclosure provides such predictive methods which assess one or more of the biomarkers described herein in a serum sample from the subject.

The present authors herein describe biomarkers and combinations thereof, methods and products for use in such methods that are of significant value in predicting clinical outcome for cancer patients. In particular, the present authors have evaluated and identified biomarkers and combinations thereof which are of significant value in the prediction of a subject's response to cancer therapies; a clinical outcome of particular interest is prediction of the subject's response to an agent capable of inhibiting or reversing EMT, in particular the subject's response to Axl kinase inhibitors such as BGB324.

Accordingly, in one facet of the present disclosure the authors have identified biomarkers and combinations thereof of significant value in the prediction of response to cancer therapies for a subject having, suspected of having, or diagnosed with acute myelocytic leukemia (AML).

In a first aspect of this facet the present disclosure provides a method of predicting a cancer-related outcome in a subject, the method comprising assessing the activity, expression, or amount of one or more biomarker in the subject, or in a sample derived from the subject; wherein the one or more biomarker is selected from the group consisting of: Axl, FGF-21, RAGE, CEA, FSH, MMP-10, Omentin, CA-19-9, LH, Haptoglobin, NAP-2, IgE, and/or CD40-L; and, wherein the subject has, is suspected of having, or has been diagnosed with acute myelocytic leukemia (AML).

In some embodiments the method comprises assessing the activity, expression, or amount of one or more biomarker in the subject, or in a sample derived from the subject, to obtain a sample profile of the one or more biomarker; and, making a prediction based on the sample profile of the one or more biomarker.

In some embodiments the prediction is made by comparing the sample profile to a control profile. In some such embodiments the sample profile is obtained before the subject is contacted with or administered an agent capable of inhibiting or reversing EMT or a chemotherapeutic agent.

In embodiments in which the sample profile is obtained before the subject is contacted with or administered an agent capable of inhibiting or reversing EMT or a chemotherapeutic agent, the control profile may be: (i) obtained from a population of control subjects having AML; (ii) obtained from a control subject or population of control subjects having AML and previously found to lack susceptibility to treatment with an agent capable of inhibiting or reversing EMT or a chemotherapeutic agent; (iii) a predetermined profile of “average, median, or mean” or “standard ranges” of biomarker expression, activity, or amount values obtained from a control subject or population of control subjects having AML and previously found to lack susceptibility to treatment with an agent capable of inhibiting or reversing EMT or a chemotherapeutic agent; (iv) obtained from a control sample having a known “average, median, or mean” value of biomarkers indicative of a control subject or population of control subjects having AML and previously found to lack susceptibility to treatment with an agent capable of inhibiting or reversing EMT or a chemotherapeutic agent; (v) a predetermined profile of biomarker expression, activity, or amount “threshold” values obtained from a control subject or population of control subjects having AML and previously found to be susceptible to treatment with an agent capable of inhibiting or reversing EMT or a chemotherapeutic agent; or (vi) obtained from a control sample having a known “threshold” value of biomarkers indicative of a control subject or population of control subjects having AML and previously found to be susceptible to treatment with an agent capable of inhibiting or reversing EMT or a chemotherapeutic agent.

In embodiments in which the sample profile is obtained before the subject is contacted with or administered an agent capable of inhibiting or reversing EMT or a chemotherapeutic agent, the one or more biomarker may include at least one of: Haptoglobin, NAP-2, IgE, and/or CD40-L; wherein a higher activity, expression, or amount of one or more of said biomarkers in the sample profile as compared with the control profile is indicative of susceptibility to treatment with an agent capable of inhibiting or reversing EMT or a chemotherapeutic agent. In other embodiments, the one or more biomarker may include at least one of: Axl, FGF-21, RAGE, CEA, FSH, MMP-10, Omentin, CA-19-9, and/or LH; wherein a lower activity, expression, or amount of one or more of said biomarkers in the sample profile as compared with the control profile is indicative of susceptibility to treatment with an agent capable of inhibiting or reversing EMT or a chemotherapeutic agent.

In some embodiments in which the sample profile is obtained before the subject is contacted with or administered an agent capable of inhibiting or reversing EMT or a chemotherapeutic agent, the one or more biomarker may comprise Axl. In other embodiments the one or more biomarker may comprise Axl and at least 1, 2, 3, 4, 5, 6, 7, 8, 9, 10, or more further biomarkers selected from: FGF-21, RAGE, CEA, FSH, MMP-10, Omentin, CA-19-9, LH, Haptoglobin, NAP-2, IgE, and/or CD40-L.

In some embodiments the sample profile is obtained after contacting the subject with an agent capable of inhibiting or reversing EMT or a chemotherapeutic agent. In some such embodiments the control profile is obtained from the same subject prior to contacting said subject with the agent capable of inhibiting or reversing EMT or a chemotherapeutic agent.

In some embodiments in which the sample profile is obtained after contacting the subject with an agent capable of inhibiting or reversing EMT or a chemotherapeutic agent, wherein an increase in the activity, expression, or amount of one or more biomarker in the sample profile as compared with the control profile is indicative of higher susceptibility to treatment with an agent capable of inhibiting or reversing EMT or a chemotherapeutic agent. In some such embodiments the one or more biomarker includes at least Axl.

In some embodiments the method comprises determining the amount of at least 2, 3, 4, 5, 6, 7, 8, 9, 10, 11, 12, 13, 14, 15, 20 of said biomarkers in the subject, or sample derived from the subject. In some embodiments assessing the expression or amount of one or more biomarker in the subject, or in a sample derived from the subject, comprises: contacting a sample, or an extract from the sample, with at least 1, 2, 3, 4, 5, 6, 7, 8, 9, 10 or more specific binding members, each of which selectively binds to a respective biomarker protein; and detecting and/or quantifying formation of complex formed by said specific binding member and said biomarker protein. In some embodiments the specific binding member comprises an antibody molecule or binding fragment thereof.

In some embodiments the agent capable of inhibiting or reversing EMT or chemotherapeutic agent is an Axl inhibitor. Preferably the Axl inhibitor is BGB324/R428/bemcentinib, or an anti-Axl antibody described in WO2015/193428, WO2015/193430, WO2016/097370, or WO2016/166296.

In some embodiments the agent capable of inhibiting or reversing EMT is administered in combination with a further cancer treatment. In some preferred embodiments the further cancer treatment is cytarabine or decitabine.

In preferred embodiments the subject is mammalian, more preferably human. In preferred embodiments the sample is a blood, serum, or plasma sample, most preferably a serum sample.

In some embodiments the method is performed in vitro or ex vivo.

In another aspect of this facet the disclosure provides methods of selecting subjects having, suspected of having, or diagnosed with acute myelocytic leukemia (AML) for treatment with an agent capable of inhibiting or reversing EMT or a chemotherapeutic agent comprising: identifying subjects susceptible to treatment with an agent capable of inhibiting or reversing EMT or a chemotherapeutic agent using a predictive method as outlined above; and selecting thus identified subjects for treatment. In some embodiments the method is a method of selecting subjects having, suspected of having, or diagnosed with acute myelocytic leukemia (AML) for continued treatment with an agent capable of inhibiting or reversing EMT or a chemotherapeutic agent, the method comprising: identifying subjects susceptible to treatment with an agent capable of inhibiting or reversing EMT or a chemotherapeutic agent using a predictive method as outlined above; and selecting thus identified subjects for continued treatment.

A further aspect of this facet of the disclosure relates to a diagnostic kit or test device comprising: 1, 2, 3, 4, 5, 6, 7, 8, 9, 10 or more specific binding members, each of which selectively binds to a biomarker selected from the group consisting of: Axl, FGF-21, RAGE, CEA, FSH, MMP-10, Omentin, CA-19-9, LH, Haptoglobin, NAP-2, IgE, and/or CD40-L; and one or more reagents for detecting said one or more specific binding members or one or more reagents for detecting and/or quantifying formation of a complex formed by said specific binding member and said biomarker. In some embodiments the diagnostic kit or test device is for use in a method of predicting a cancer-related outcome in a subject. In some embodiments the method is a predictive method as outlined above.

A further aspect of this facet of the disclosure relates to use of one or more of: Axl, FGF-21, RAGE, CEA, FSH, MMP-10, Omentin, CA-19-9, LH, Haptoglobin, NAP-2, IgE, and/or CD40-L, as a biomarker for determining if a subject is susceptible to treatment with an agent capable of inhibiting or reversing EMT or a chemotherapeutic agent. In some embodiments the use is as a biomarker in a predictive method as outlined above.

Further facets of the disclosure relate to biomarkers and combinations thereof, methods and products for use in such methods that are of significant value in predicting clinical outcome for subjects having, suspected of having, or diagnosed with myelodysplastic syndromes (MDS), melanoma, triple-negative breast cancer (TNBC), non-small cell lung cancer (NSCLC), and lung adenocarcinoma. In particular, the present authors have evaluated and identified biomarkers and combinations thereof which are of significant value in the prediction of a subject having one of these cancers' response to cancer therapies; a clinical outcome of particular interest is prediction of the subject's response to an agent capable of inhibiting or reversing EMT, in particular the subject's response to Axl kinase inhibitors such as BGB324. Each of these facets has aspects corresponding to those set out above for AML; these and other aspects are disclosed in more detail below.

The disclosure includes the combination of the aspects and preferred features described except where such a combination is clearly impermissible or expressly avoided.

### DETAILED DESCRIPTION OF THE DISCLOSURE

Facets, aspects and embodiments of the present disclosure will now be discussed with reference to the accompanying figures. Further aspects and embodiments will be apparent to those skilled in the art. All documents mentioned in this text are incorporated herein by reference.

The present authors herein describe biomarkers and combinations thereof, as well as methods and products for use in such methods that are of significant value for the prediction of cancer-related outcomes. In particular, the present authors have identified and evaluated serum-based markers, and combinations thereof, which are of significant value in the prediction of a subject's response to cancer therapies. In some embodiments the cancer therapy is an agent capable of inhibiting or reversing EMT, for example an Axl inhibitor or Akt3 inhibitor.

Biomarkers in Predictive Methods

Heterogeneous Patient Populations

Whilst cancers are categorised into classes which share broad characteristics, these classes may be composed of heterogeneous sub-populations. Patients in different sub-populations may have different responses to the same cancer treatment.

For example, acute myeloid leukemia (AML) is known to be a heterogeneous cancer with existing cell lines such as MOLM13 and Mv4-11 that have a low IC50 to Axl inhibitors such as BGB324 (i.e. cells that respond well to treatment with Axl-inhibitors, so-called “responder” cell lines), and cells such as Kasumi, OCI-M1 and OCI-AML5 that have a high 1050 to Axl inhibitors such as BGB324 (i.e. cells that respond poorly to treatment with Axl-inhibitors, so-called “non-responder” cell lines; Ben-Batalla et al, 2013). Similarly, the present authors have found that in AML in humans there are subjects who respond to or benefit from treatment with Axl inhibitors such as BGB324 (so-called “responder” subjects), and subjects who do not respond to or benefit from treatment with Axl inhibitors such as BGB324 (so-called “non-responder” subjects).

By identifying biomarkers that reliably distinguish these responder and non-responder subjects it is possible to use the biomarkers to predict the clinical outcome for subjects.

Predictive Methods

The term “predictive method” as used herein means a method that enables a determination of the likelihood of a subject being susceptible or responsive to treatment with a particular agent/regimen. Such predictive methods provide information on the likely outcome of a particular treatment regimen, for example, the likelihood of a subject responding to said treatment, and/or information as to how aggressively an individual should be treated within a particular treatment regimen, and/or how aggressively an individual should be treated with conventional therapeutic methods such as radiation/chemotherapy. The predictive methods described herein therefore have important applications in the field of personalised medicines.

The predictive methods described herein enable predictions regarding the susceptibility of human or animal subjects diagnosed with a disease to treatment with an agent capable of inhibiting or reversing EMT or a chemotherapeutic agent. More specifically, the disease of interest may be cancer, including acute myelocytic leukemias (AMLs) or myelodysplastic syndromes (MDSs), breast, lung, prostate, ovarian, colorectal, glioma, melanoma, gastric, head and neck, renal, pancreatic, uterine, hepatic, bladder, and endometrial cancers, as well as other leukemias. AML and MDS are of particular interest, as are lung cancers such as non-small cell lung carcinoma, in particular lung adenocarcinoma.

The authors herein describe biomarkers and combinations thereof that are of significant value in predicting clinical outcome for cancer patients. In particular, the present authors have evaluated and identified serum-based markers which are of significant value in the prediction of a subject's response to cancer therapies. Also contemplated are panels comprising two or more of the biomarkers, offering increased sensitivity and reliability in predicting cancer-related outcomes, for example susceptibility to treatment with an agent capable of inhibiting or reversing EMT or a chemotherapeutic agent.

Solid and Liquid Tumours

Tumours may be termed “solid” or “liquid” dependent upon where in the body they grow. The majority of cancers are caused by solid tumours present as a mass of cells in particular organ or tissue. Common “solid” tumour cancers include breast, lung, prostate, and colon cancers. Liquid tumours are those which develop in the blood or bone marrow and which can travel to any part of the body, for example leukaemia or myeloma. “Liquid” tumour cancers may also be referred to as “blood cancers”.

The authors have observed that different markers are predictive of a subject's response to cancer therapies in solid as compared to liquid tumour cancers.

Accordingly, the disclosure provides methods of predicting a cancer-related outcome in a subject having, suspected of having, or diagnosed with a solid tumour cancer, the method comprising assessing the activity, expression, or amount of one or more of the biomarkers described herein in the subject, or in a sample derived from the subject.

The disclosure also provides methods of predicting a cancer-related outcome in a subject having, suspected of having, or diagnosed with a liquid tumour cancer, the method comprising assessing the activity, expression, or amount of one or more of the biomarkers described herein in the subject, or in a sample derived from the subject.

In some embodiments the cancer-related outcome is susceptibility to treatment with an agent capable of inhibiting or reversing EMT or a chemotherapeutic agent. In some preferred embodiments the cancer-related outcome is susceptibility to treatment with an Axl inhibitor.

Solid tumour cancers include, for example, breast, renal, endometrial, ovarian, thyroid, and non-small cell lung carcinoma, melanoma, prostate carcinoma, sarcoma, gastric cancer and uveal melanoma. Liquid tumour cancers include, for example, leukemias (particularly myeloid leukemias) and lymphomas.

Facet 1: Acute Myelocytic Leukemia (AML)

Acute myeloid leukemia (AML) is a clonal disease of hematopoietic progenitors that is characterized by numerous heterogeneous genetic changes that alter the cells' normal mechanisms of proliferation, differentiation and cell death (Burnett et al., 2011). Currently, most AML patients are treated with chemotherapy such as Cytarabine. Apart from the BCR-Abl targeting drug Gleevec for treatment of chronic myelogenous leukemia (CML) and the differentiation-inducing drug ATRA for treatment of acute promyelocytic leukemia (APL), targeted therapy has so far been extensively lacking in the field of leukemia. This despite AML being a well characterized disease with several mutated oncogenes that could potentially be therapeutic targets (Haferlach, 2008).

Accordingly, robust biomarkers for predicting cancer-related outcomes in a subject, for example susceptibility to treatment with an agent capable of inhibiting or reversing EMT or a chemotherapeutic agent, would be useful in identifying subjects who are most likely to respond to or benefit from treatment with a particular agent/regimen.

A “responder” AML cell line is a cell line with a low IC₅₀ for Axl inhibitors, for example the small molecule Axl kinase inhibitor BGB324/R428/bemcentinib; examples include the MOLM13 and Mv4-11 cell lines, with an IC₅₀ of 0.45 μM and 0.14 μM, respectively. Correspondingly, a “non-responder” AML cell line is a cell line with a high IC₅₀ for Axl inhibitors, for example the small molecule Axl kinase inhibitor BGB324/R428/bemcentinib; examples are the Kasumi and OCI-M1 cell lines, with an IC₅₀ of 1.2 μM and 1.8 μM, respectively.

A “responder” subject as described herein is a subject who is likely to, or who does, respond to treatment (or benefit from treatment) with an agent capable of inhibiting or reversing EMT or a chemotherapeutic agent, for example the small molecule Axl kinase inhibitor BGB324/R428/bemcentinib when administered either as a single agent or in combination with another cancer treatment.

Correspondingly, a “non-responder” subject as described herein is a subject who is not likely to, or who does not, respond to treatment (or benefit from treatment) with an agent capable of inhibiting or reversing EMT or a chemotherapeutic agent, for example the small molecule Axl kinase inhibitor BGB324/R428/bemcentinib when administered either as a single agent or in combination with another cancer treatment.

To “respond to treatment” or “benefit from treatment” as used herein means to experience an overall clinical benefit from the treatment. This overall clinical benefit can be any of: prolonged survival, partial or complete disease remission (for example, as assessed by % bone marrow myeloblasts and/or normal maturation of cell lines), slowing or absence of disease progression (for example, as assessed by change in % bone marrow myeloblasts), improved quality of life (for example, as assessed using a health-related quality of life questionnaire such as a Functional Assessment of Cancer Therapy (FACT) questionnaire), progression-free survival, hematologic improvement (for example: increased blood haemoglobin, platelet count, and/or neutrophil count), bone marrow response (for example: bone marrow with 5% myeloblasts; 30%, 40%, 50% or more reduction in bone marrow myeloblasts; absence of circulating myeloblasts and myeloblasts with Auer rods; absence of extramedullary disease), hematologic recovery (for example: ≥1 g/dL haemoglobin, ≥100×10⁹/L platelets, and/or ≥1×10⁹/L neutrophils in peripheral blood), negative response for a genetic marker (for example, CEBPA, NPM1, or FLT3), or any other positive patient outcome.

Biomarkers

The term “marker” or “biomarker” is used herein to refer to a gene or protein whose expression in a subject or sample derived from a subject is altered or modulated, for example, up or down regulated, in cancer. Where the biomarker is a protein, modulation or alteration of expression encompasses modulation through different post-translational modifications.

In accordance with any aspect of this facet of the present disclosure the biomarkers described herein may be defined as follows, wherein:
an Axl biomarker comprises an amino acid sequence having at least 70%, 80%, 90%, 95%, 99% or 100% sequence identity with the full-length of the amino acid sequence disclosed at UniProt accession no: P30530-1 or P30530-2 (entry version 196) or a fragment thereof;
a Fibroblast Growth Factor 21 (FGF-21) biomarker comprises an amino acid sequence having at least 70%, 80%, 90%, 95%, 99% or 100% sequence identity with the full-length of the amino acid sequence disclosed at UniProt accession no: Q9NSA1 (entry version 145) or a fragment thereof;
a Receptor for advanced glycosylation end products (RAGE) biomarker comprises an amino acid sequence having at least 70%, 80%, 90%, 95%, 99% or 100% sequence identity with the full-length of the amino acid sequence disclosed at UniProt accession no: Q15109-1, Q15109-2, Q15109-3, Q15109-4, Q15109-5, Q15109-6, Q15109-7, Q15109-8, Q15109-9, Q15109-10 (entry version 189) or a fragment thereof;
a Carcinoembryonic Antigen (CEA) biomarker comprises an amino acid sequence having at least 70%, 80%, 90%, 95%, 99% or 100% sequence identity with the full-length of the amino acid sequence disclosed at UniProt accession no: P06731-1 or P06731-2 (entry version 179) or a fragment thereof;
a Follicle-Stimulating Hormone (FSH) biomarker comprises an amino acid sequence having at least 70%, 80%, 90%, 95%, 99% or 100% sequence identity with the full-length of the amino acid sequence disclosed at UniProt accession no: P01225 (entry version 189) or a fragment thereof;
a Matrix Metalloproteinase-10 (MMP-10) biomarker comprises an amino acid sequence having at least 70%, 80%, 90%, 95%, 99% or 100% sequence identity with the full-length of the amino acid sequence disclosed at UniProt accession no: P09238 (entry version 185) or a fragment thereof;
an Omentin biomarker comprises an amino acid sequence having at least 70%, 80%, 90%, 95%, 99% or 100% sequence identity with the full-length of the amino acid sequence disclosed at UniProt accession no: Q8WWA0 (entry version 127) or a fragment thereof;
a Cancer Antigen 19-9 (CA-19-9) biomarker comprises an amino acid sequence having at least 70%, 80%, 90%, 95%, 99% or 100% sequence identity with the full-length of the amino acid sequence disclosed at UniProt accession no: Q9BXJ9-1 or Q9BXJ9-2 (entry version 160) or a fragment thereof;
a Luteinizing Hormone (LH) biomarker comprises an amino acid sequence having at least 70%, 80%, 90%, 95%, 99% or 100% sequence identity with the full-length of the amino acid sequence disclosed at UniProt accession no: P01229 (entry version 179) or a fragment thereof;
a Haptoglobin (HP) biomarker comprises an amino acid sequence having at least 70%, 80%, 90%, 95%, 99% or 100% sequence identity with the full-length of the amino acid sequence disclosed at UniProt accession no: P00738-1 or P00738-2 (entry version 200) or a fragment thereof;
a Neutrophil Activating Peptide 2 (NAP-2) biomarker comprises an amino acid sequence having at least 70%, 80%, 90%, 95%, 99% or 100% sequence identity with the full-length of the amino acid sequence disclosed at UniProt accession no: P02775 (entry version 191) or a fragment thereof;
an Immunoglobulin E (IgE) biomarker comprises an amino acid sequence having at least 70%, 80%, 90%, 95%, 99% or 100% sequence identity with the full-length of the amino acid sequence disclosed at UniProt accession no: P01854 (entry version 162) or a fragment thereof; and/or
a CD40 Ligand (CD40-L) biomarker comprises an amino acid sequence having at least 70%, 80%, 90%, 95%, 99% or 100% sequence identity with the full-length of the amino acid sequence disclosed at UniProt accession no: P29965 (entry version 199) or a fragment thereof.

The biomarkers described herein may also be defined as a nucleic acid (either DNA or RNA) having at least 70%, 80%, 90%, 95%, 99% or 100% sequence identity with the full-length nucleic acid sequence encoding any of the above amino acid sequences.

Identity may be as defined using sequence comparisons made using FASTA and FASTP (see Pearson & Lipman, 1988. Methods in Enzymology 183: 63-98). Parameters are preferably set, using the default matrix, as follows: Gapopen (penalty for the first residue in a gap): −12 for proteins/-16 for DNA; Gapext (penalty for additional residues in a gap): −2 for proteins/-4 for DNA; KTUP word length: 2 for proteins/6 for DNA.

Pre-Treatment Predictive Biomarkers and Methods

By analysing patient data obtained from clinical trial BGBC003 (NCT02488408), the authors have discovered that, prior to treatment of subjects having AML, certain of the biomarkers described herein are present in the serum of “responder” subjects at higher levels than in the serum of “non-responder” subjects—see Example 1. Similarly, prior to treatment of the subject certain of the biomarkers described herein are present in the serum of “responder” subjects at lower levels than in the serum of “non-responder” subjects. Thus, by assessing the activity, expression, or amount of one or more of the biomarkers described herein prior to treatment with an agent capable of inhibiting or reversing EMT or a chemotherapeutic agent it is possible to determine the likelihood of that subject being susceptible or responsive to treatment with said agent.

Accordingly, in a first aspect, the disclosure provides a method of predicting a cancer-related outcome in a subject having, suspected of having, or diagnosed with acute myelocytic leukemia (AML), the method comprising assessing the activity, expression, or amount of one or more of the biomarkers described herein in the subject, or in a sample derived from the subject.

In some embodiments the one or more biomarker is selected from the group consisting of: Axl, Fibroblast Growth Factor 21 (FGF-21), Receptor for advanced glycosylation end products (RAGE), Carcinoembryonic Antigen (CEA), Follicle-Stimulating Hormone (FSH), Matrix Metalloproteinase-10 (MMP-10), Omentin, Cancer Antigen 19-9 (CA-19-9), Luteinizing Hormone (LH), Haptoglobin (HP), Neutrophil Activating Peptide 2 (NAP-2), Immunoglobulin E (IgE), and/or CD40 Ligand (CD40-L).

In some embodiments the one or more biomarker is, or includes, Axl.

A “cancer-related outcome” as described herein is a clinical prediction or prognosis associated with cancer. In some embodiments the cancer-related outcome is susceptibility to treatment with an agent capable of inhibiting or reversing EMT or a chemotherapeutic agent.

In some embodiments the method comprises assessing the activity, expression, or amount of one or more of the biomarkers described herein in the subject, or in a sample derived from the subject, to obtain a sample profile of the one or more biomarker; and, making a prediction based on the sample profile of the one or more biomarker.

A “sample profile” as used herein is a profile of numerical values or numerical ranges corresponding to the activity, expression, or amount of respective biomarkers determined in the subject or sample derived from the subject.

In some embodiments of the method the prediction is made by comparing the sample profile to a control profile. A “control profile” as used herein is a profile of numerical values or numerical ranges corresponding to the activity, expression, or amount of the same biomarkers as for the sample profile, determined in a control subject or population of control subjects. Suitable control profiles are described in more detail below.

Prior to treatment of a subject, certain of the biomarkers described herein have been found to exhibit up-regulated expression in “responder” subjects—that is, certain of the biomarkers described herein have been found to be present in the serum of “responder” subjects at higher levels than in the serum of “non-responder” subjects. Similarly, prior to treatment of a subject, certain of the biomarkers described herein have been found to exhibit down-regulated expression in “responder” subjects—that is, certain of the biomarkers described herein have been found to be present in the serum of “responder” subjects at lower levels than in the serum of “non-responder” subjects.

This difference in expression level between “responder” and “non-responder” subjects can also be expressed in the opposite way. That is to say, prior to treatment of a subject, certain of the biomarkers described herein have been found to exhibit down-regulated expression in “non-responder” subjects—that is, certain of the biomarkers described herein have been found to be present in the serum of “non-responder” subjects at lower levels than in the serum of “responder” subjects. Similarly, prior to treatment of a subject, certain of the biomarkers described herein have been found to exhibit up-regulated expression in “non-responder” subjects—that is, certain of the biomarkers described herein have been found to be present in the serum of “non-responder” subjects at higher levels than in the serum of “responder” subjects.

The discovery of these differential patterns of expression mean that, by assessing the activity, expression, or amount of one or more of the biomarkers described herein prior to treatment of a subject with an agent, it is possible to determine the likelihood of that subject being susceptible or responsive to treatment with said agent. Accordingly, in particularly preferred embodiments the sample profile is obtained before the subject is contacted with or administered the agent capable of inhibiting or reversing EMT or a chemotherapeutic agent.

A subject who is “susceptible to treatment” with an agent capable of inhibiting or reversing EMT or a chemotherapeutic agent is one who is likely to, or who does, respond to treatment (or benefit from treatment) with an agent capable of inhibiting or reversing EMT or a chemotherapeutic agent. That is, a subject who is “susceptible to treatment” with an agent is a “responder” subject for that agent, as defined above. A prediction that is “indicative of susceptibility” is one which suggests or points towards the likelihood that a subject will respond to or benefit from treatment.

In some embodiments the predictive methods of the disclosure comprise assessing the activity, expression, or amount of one or more of the biomarkers described herein in the subject, or in a sample derived from the subject, to obtain a sample profile of the one or more biomarker; and, making a prediction based on the sample profile of the one or more biomarker. In some embodiments of the method the prediction is made by comparing the sample profile to a control profile, wherein the control profile is a profile of numerical values or numerical ranges corresponding to the activity, expression, or amount of the same biomarkers as for the sample profile, determined in a control subject or population of control subjects.

In some embodiments the control profile may be obtained from a population of control subjects having AML. In some embodiments the control profile may be obtained from a population of control subjects not having AML. In other embodiments the control profile may be obtained from a control subject or population of control subjects having AML and previously found to lack susceptibility to treatment with an agent capable of inhibiting or reversing EMT or a chemotherapeutic agent (that is, non-responder subjects).

In some embodiments the control profile may a predetermined profile of biomarker expression, activity, or amount values, for example a profile of “mean” values, “threshold” values, or “standard ranges” of values. This predetermined profile of biomarker expression, activity, or amount values can be obtained, for example, from a control subject or population of control subjects having AML, a control subject or population of control subjects not having AML, or a control subject or population of control subjects having AML and previously found to lack susceptibility to treatment with an agent capable of inhibiting or reversing EMT or a chemotherapeutic agent (that is, non-responder subjects).

In some other embodiments the control profile may be obtained from a control subject or population of control subjects having AML and previously found to be susceptible to treatment with an agent capable of inhibiting or reversing EMT or a chemotherapeutic agent (that is, responder subjects). In some embodiments the control profile may a predetermined profile of biomarker expression, activity, or amount values, for example a profile of “threshold”, values or “standard ranges” of values. This predetermined profile of biomarker expression, activity, or amount values can be obtained, for example, from a control subject or population of control subjects having AML and previously found to be susceptible to treatment with an agent capable of inhibiting or reversing EMT or a chemotherapeutic agent (that is, responder subjects).

In some other embodiments the control profile may be obtained from a control sample having a known amount (for example a “threshold” amount) of the same biomarkers as will be determined in the sample profile. This threshold amount can be obtained, for example, from non-responder or responder subjects as outlined above.

The skilled person is readily able to determine suitable “mean”, “threshold”, or “standard range” values for a particular biomarker.

For example, a “mean” value may be determined by: assessing the activity, expression, or amount of a particular biomarker in a population of subjects having (or not having) AML; and, averaging the biomarker activity, expression, or amount determined to determine a “mean” value.

For example, a “mean” value may be determined by: assessing the activity, expression, or amount of a particular biomarker in a population of subjects; determining which subjects in the population are susceptible to treatment with an agent capable of inhibiting or reversing EMT or a chemotherapeutic agent; and, averaging the biomarker activity, expression, or amount determined in subjects found to be susceptible to treatment with an agent capable of inhibiting or reversing EMT or a chemotherapeutic agent (that is, responder subjects) to determine a “mean” value.

For example, a “threshold” value may be determined by: assessing the activity, expression, or amount of a particular biomarker in a population of subjects; determining which subjects in the population are susceptible to treatment with an agent capable of inhibiting or reversing EMT or a chemotherapeutic agent; and, selecting as appropriate the highest or lowest biomarker activity, expression, or amount determined in a subject found to be susceptible to treatment with an agent capable of inhibiting or reversing EMT or a chemotherapeutic agent (that is, a responder subject) as the “threshold value”.

For example, a “standard range” of values may be determined by: assessing the activity, expression, or amount of a particular biomarker in a population of subjects having (or not having) AML; and, determining a “standard range” of values based on this assessment.

For example, a “standard range” of values may be determined by: assessing the activity, expression, or amount of a particular biomarker in a population of subjects; determining which subjects in the population are susceptible to treatment with an agent capable of inhibiting or reversing EMT or a chemotherapeutic agent; and, determining a “standard range” of values for subjects found to be susceptible to treatment with an agent capable of inhibiting or reversing EMT or a chemotherapeutic agent (that is, a responder subject) based on this assessment.

Where the control profile is a predetermined profile of biomarker expression, activity, or amount values, this may be provided as a “look-up table” or data record. Predetermined control profiles of this type may be a profile of numerical values or numerical ranges corresponding to a measure of central tendency (such as an average, median or mean) amount of the respective biomarkers determined in a plurality of control subjects (for example non-responder or responder subjects as outlined above). The skilled person is readily able to determine both a measure of central tendency and a measure of variability within a population using standard protein quantification techniques and suitable statistical tools.

Accordingly, in embodiments in which the control profile is: obtained from a population of control subjects having AML; obtained from a population of control subjects not having AML; obtained from a control subject or population of control subjects having AML and previously found to lack susceptibility to treatment with an agent capable of inhibiting or reversing EMT or a chemotherapeutic agent (that is non-responder subjects); a predetermined profile of biomarker expression, activity, or amount values (for example a profile of “average, median, or mean” values or “standard ranges” of values) obtained from non-responder subjects; obtained from a control sample having a known “average, median, or mean” value of biomarkers indicative of non-responder subjects; a predetermined profile of biomarker expression, activity, or amount “threshold” values obtained from responder subjects; or, obtained from a control sample having a known “threshold” value of biomarkers indicative of responder subjects; then, a higher activity, expression, or amount of one or more of the biomarkers described herein in a sample profile as compared with the control profile may be indicative of susceptibility to treatment with an agent capable of inhibiting or reversing EMT or a chemotherapeutic agent. Similarly, a lower activity, expression, or amount of one or more of the biomarkers described herein in the sample profile as compared with a control profile may be indicative of susceptibility to treatment with an agent capable of inhibiting or reversing EMT or a chemotherapeutic agent.

In some such embodiments, the method may comprise assessing the activity, expression, or amount of one or more of biomarker selected from: Haptoglobin, NAP-2, IgE, and/or CD40-L; wherein a higher activity, expression, or amount of one or more of said biomarkers in the sample profile as compared with the control profile is indicative of susceptibility to treatment with an agent capable of inhibiting or reversing EMT or a chemotherapeutic agent.

In some such embodiments, the method may comprise assessing the activity, expression, or amount of one or more of biomarker selected from: Axl, FGF-21, RAGE, CEA, FSH, MMP-10, Omentin, CA-19-9, and/or LH; wherein a lower activity, expression, or amount of one or more of said biomarkers in the sample profile as compared with the control profile is indicative of susceptibility to treatment with an agent capable of inhibiting or reversing EMT or a chemotherapeutic agent.

In some such embodiments: a lower activity, expression, or amount of Axl; and/or a lower activity, expression, or amount of FGF-21; and/or a lower activity, expression, or amount of RAGE; and/or a lower activity, expression, or amount of CEA; and/or a lower activity, expression, or amount of FSH; and/or a lower activity, expression, or amount of MMP-10; and/or a lower activity, expression, or amount of Omentin; and/or a lower activity, expression, or amount of CA-19-9; and/or a lower activity, expression, or amount of LH; and/or a higher activity, expression, or amount of Haptoglobin; and/or a higher activity, expression, or amount of NAP-2; and/or a higher activity, expression, or amount of IgE; and/or a higher activity, expression, or amount of CD40-L; in the sample profile as compared with the control profile is indicative of susceptibility to treatment with an agent capable of inhibiting or reversing EMT or a chemotherapeutic agent.

In embodiments in which the control profile is: obtained from a control subject or population of control subjects having AML and previously found to be susceptible to treatment with an agent capable of inhibiting or reversing EMT or a chemotherapeutic agent (that is, responder subjects); then, if the expression, activity, or amount values in the sample profile are above a “threshold” value typical of a responder subject (that is, a subject having AML and previously found to be susceptible to treatment with an agent capable of inhibiting or reversing EMT or a chemotherapeutic agent) this is indicative of susceptibility to treatment with an agent capable of inhibiting or reversing EMT or a chemotherapeutic agent. Similarly, if the expression, activity, or amount values in the sample profile are below the “threshold” value typical of a responder subject (that is, a subject having AML and previously found to be susceptible to treatment with an agent capable of inhibiting or reversing EMT or a chemotherapeutic agent) this is indicative of a lack of susceptibility to treatment with an agent capable of inhibiting or reversing EMT or a chemotherapeutic agent.

In embodiments in which the control profile is: a predetermined profile of “standard ranges” of biomarker expression, activity, or amount values obtained from responder subjects; then, if the expression, activity, or amount values in the sample profile are within the “standard range” of values typical of a responder subject (that is, a subject having AML and previously found to be susceptible to treatment with an agent capable of inhibiting or reversing EMT or a chemotherapeutic agent) this is indicative of susceptibility to treatment with an agent capable of inhibiting or reversing EMT or a chemotherapeutic agent. Similarly, if the expression, activity, or amount values in the sample profile are outside the “standard range” of values typical of a responder subject (that is, a subject having AML and previously found to be susceptible to treatment with an agent capable of inhibiting or reversing EMT or a chemotherapeutic agent) this is indicative of a lack of susceptibility to treatment with an agent capable of inhibiting or reversing EMT or a chemotherapeutic agent.

In embodiments in which the control profile is: a predetermined profile of “standard ranges” of biomarker expression, activity, or amount values obtained from non-responder subjects; then, if the expression, activity, or amount values in the sample profile are within the “standard range” of values typical of a non-responder subject (that is, a subject having AML and previously found to lack susceptibility to treatment with an agent capable of inhibiting or reversing EMT or a chemotherapeutic agent) this is indicative of a lack of susceptibility to treatment with an agent capable of inhibiting or reversing EMT or a chemotherapeutic agent. Similarly, if the expression, activity, or amount values in the sample profile are outside the “standard range” of values typical of a non-responder subject (that is, a subject having AML and previously found to lack susceptibility to treatment with an agent capable of inhibiting or reversing EMT or a chemotherapeutic agent) this may be indicative of susceptibility to treatment with an agent capable of inhibiting or reversing EMT or a chemotherapeutic agent.

In embodiments in which the control profile is: obtained from a population of control subjects not having AML; then, if the expression, activity, or amount values in the sample profile are outside a “standard range” of values typical of subjects not having AML, this is indicative of susceptibility to treatment with an agent capable of inhibiting or reversing EMT or a chemotherapeutic agent. Similarly, if the expression, activity, or amount values in the sample profile are within a “standard range” of values typical of subjects not having AML, this is indicative of a lack of susceptibility to treatment with an agent capable of inhibiting or reversing EMT or a chemotherapeutic agent. In such cases, an expression, activity or amount value may be outside the “standard range” of values when it is either above the upper limit of normal or below the lower limit of normal for a given biomarker. The upper limit of normal and lower limit of normal are the limits of the normal distribution of a given biomarker in a population of control subjects not having AML.

The skilled person is readily able to determine whether the sample profile is indicative of susceptibility or lack of susceptibility to treatment with an agent capable of inhibiting or reversing EMT or a chemotherapeutic agent using suitable statistical tools and comparison with the appropriate control.

In some embodiments the sample profile may be compared with two or more control profiles as described above.

As used herein, the term “one or more” means 1, 2, 3, 4, 5, 6, 7, 8, 9, 10, 11, 12, 13, 14, 15, 16, 17, 18, 19, or 20 of the biomarkers described herein. In some embodiments the one or more biomarker is Axl. In some embodiments the one or more biomarker is FGF-21. In some embodiments the one or more biomarker is RAGE. In some embodiments the one or more biomarker is CEA. In some embodiments the one or more biomarker is FSH. In some embodiments the one or more biomarker is MMP-10. In some embodiments the one or more biomarker is Omentin. In some embodiments the one or more biomarker is CA-19-9. In some embodiments the one or more biomarker is LH. In some embodiments the one or more biomarker is Haptoglobin. In some embodiments the one or more biomarker is NAP-2. In some embodiments the one or more biomarker is IgE. In some embodiments the one or more biomarker is CD40-L.

In some embodiments the method may comprise assessing the activity, expression, or amount of at least 2, 3, 4, 5, 6, 7, 8, 9, 10, 11, 12, 13, 14, 15, 20 of said biomarkers in the subject, or in a sample derived from the subject. In some embodiments the method may comprise assessing the activity, expression, or amount of Axl and at least 1, 2, 3, 4, 5, 6, 7, 8, 9, 10, 11, 12, 13, 14, 15, 20 further biomarkers described herein in the subject, or in a sample derived from the subject.

Subject

A “subject” as referred to herein is any species of animal classified as a mammal, and includes but is not restricted to, domestic and farm animals, primates and humans. In some preferred embodiments the subject is a human of any sex or race. In some embodiments, the human is an adult human.

In some embodiments the predictive methods described herein are performed before the subject receives a treatment or course of treatment for AML. In other embodiments the predictive method is performed during or after a treatment or course of treatment for AML. In some embodiments the treatment or course of treatment for AML is an agent capable of inhibiting or reversing EMT. In other embodiments the treatment or course of treatment for AML is a chemotherapeutic treatment that is not an agent capable of inhibiting or reversing EMT.

In some embodiments of the predictive methods described herein the subject has previously been treated with an agent capable of inhibiting or reversing EMT or a chemotherapeutic agent. In some embodiments the subject has previously been treated with an agent selected from: a PHGDH inhibitor, a Slfn11 inhibitor, an Axl inhibitor, or an Akt3 inhibitor.

In some embodiments of the predictive methods described herein the subject has previously been treated with an agent that is different to the agent capable of inhibiting or reversing EMT or chemotherapeutic agent for which the subject's response is being predicted. That is, the subject has previously been treated with an agent that is not the agent which the predictive method is determining the subject's susceptibility to. Accordingly, in some embodiments, the subject has not previously been treated with an agent capable of inhibiting or reversing EMT or a chemotherapeutic agent. In some preferred embodiments, the subject has not previously been treated with an Axl inhibitor. In other preferred embodiments, the subject has not previously been treated with an Akt3 inhibitor.

Post-Treatment Changes in Biomarkers

In some embodiments of this aspect of the disclosure, the predictive method further comprises a subsequent step of assessing the activity, expression, or amount of one or more biomarker in the subject, or in a sample derived from the subject, to obtain a second sample profile of one or more of the biomarkers described herein after the subject has received a treatment or course of treatment an agent capable of inhibiting or reversing EMT or a chemotherapeutic agent.

That is, in some embodiments, the predictive method comprises:
(a) determining if a subject is susceptible to treatment with an agent capable of inhibiting or reversing EMT or a chemotherapeutic agent by assessing the activity, expression, or amount of one or more biomarker in the subject, as outlined above;
(b) administering to the subject a therapeutically effective amount of an agent capable of inhibiting or reversing EMT or a chemotherapeutic agent; and
(c) subsequently assessing the activity, expression, or amount of one or more biomarker in the subject, or in a sample derived from the subject.

In some embodiments, the predictive method comprises:
(a) determining if a subject is susceptible to treatment with an agent capable of inhibiting or reversing EMT or a chemotherapeutic agent by assessing the activity, expression, or amount of one or more biomarker in the subject, or in a sample derived from the subject, wherein the one or more biomarker is selected from the group consisting of: Axl, FGF-21, RAGE, CEA, FSH, MMP-10, Omentin, CA-19-9, LH, Haptoglobin, NAP-2, IgE, and/or CD40-L.;
(b) administering to the subject a therapeutically effective amount of an agent capable of inhibiting or reversing EMT or a chemotherapeutic agent; and
(c) subsequently assessing the activity, expression, or amount of one or more biomarker in the subject, or in a sample derived from the subject.

In some preferred embodiments the methods comprise subsequently assessing the activity, expression, or amount of Axl in the subject, or in a sample derived from the subject. In some embodiments an increase in the activity, expression, or amount of Axl in the second sample profile as compared with a control profile after contacting the subject with a cancer therapeutic agent is indicative of higher susceptibility to treatment with an agent capable of inhibiting or reversing EMT or a chemotherapeutic agent. In some preferred embodiments the control profile is obtained from the same subject prior to treatment with an agent capable of inhibiting or reversing EMT or a chemotherapeutic agent.

Post-Treatment Predictive Biomarkers and Methods

By analysing patient data obtained from clinical trial BGBC003 (NCT02488408), the authors have discovered that, following treatment of subjects having AML with an agent capable of inhibiting or reversing EMT or a chemotherapeutic agent, the levels of certain of the biomarkers described herein increases in the serum of “responder” subjects but not in the serum of “non-responder” subjects—see Example 1. Similarly, following treatment with an agent capable of inhibiting or reversing EMT or a chemotherapeutic agent, the levels of certain of the biomarkers described herein decreases in the serum of “responder” subjects but not in the serum of “non-responder” subjects. Thus, by assessing the activity, expression, or amount of one or more of the biomarkers described herein prior to and following treatment with an agent capable of inhibiting or reversing EMT or a chemotherapeutic agent it is possible to determine or confirm that the subject is susceptible or responsive to treatment with said agent.

Accordingly, in a second aspect of this facet the disclosure provides a method of predicting a cancer-related outcome in a subject having, suspected of having, or diagnosed with acute myelocytic leukemia (AML), the method comprising assessing the activity, expression, or amount of one or more of the biomarkers described herein in a subject, or in a sample derived from the subject after the subject has been contacted with an agent capable of inhibiting or reversing EMT or a chemotherapeutic agent. In some embodiments the cancer-related outcome is susceptibility to treatment with an agent capable of inhibiting or reversing EMT or a chemotherapeutic agent.

In some embodiments the method comprises assessing the activity, expression, or amount of one or more of the biomarkers described herein in the subject, or in a sample derived from the subject, to obtain a sample profile of the one or more biomarker; and, making a prediction based on the sample profile of the one or more biomarker. In some embodiments of the method the prediction is made by comparing the sample profile to a control profile. In preferred embodiments the sample profile is obtained after contacting the subject with an agent capable of inhibiting or reversing EMT or a chemotherapeutic agent.

A “control profile” as used herein is a profile of numerical values or numerical ranges corresponding to the activity, expression, or amount of the same biomarkers as for the sample profile, determined in a control subject or population of control subjects.

In preferred embodiments of this aspect of the disclosure the control profile is obtained from the same subject prior to treatment with an agent capable of inhibiting or reversing EMT or a chemotherapeutic agent. In some embodiments the control profile is obtained from a population of control subjects having AML. In other embodiments, the control profile is a predetermined profile of biomarker expression, activity, or amount values, for example a profile of “threshold” values or “standard ranges” of values.

Accordingly, in embodiments in which the control profile is obtained from the same subject prior to treatment with an agent capable of inhibiting or reversing EMT or a chemotherapeutic agent, an increase in the activity, expression, or amount of one or more biomarker in the sample profile as compared with a control profile after contacting the subject with an agent capable of inhibiting or reversing EMT or a cancer therapeutic agent may be indicative of susceptibility to treatment with an agent capable of inhibiting or reversing EMT or a chemotherapeutic agent. In such embodiments, a decrease in the activity, expression, or amount of one or more biomarker in the sample profile as compared with a control profile after contacting the subject with a cancer therapeutic agent may be indicative of higher susceptibility to treatment with an agent capable of inhibiting or reversing EMT or a chemotherapeutic agent.

In some such embodiments, an increase in the activity, expression, or amount of one or more biomarker in the sample profile as compared with a control profile after contacting the subject with a cancer therapeutic agent may be indicative of lower susceptibility to treatment with an agent capable of inhibiting or reversing EMT or a chemotherapeutic agent. In some such embodiments, a decrease in the activity, expression, or amount of one or more biomarker in the sample profile as compared with a control profile after contacting the subject with a cancer therapeutic agent may be indicative of lower susceptibility to treatment with an agent capable of inhibiting or reversing EMT or a chemotherapeutic agent.

In some preferred embodiments the one or more biomarker includes at least Axl. In some embodiments the one or more biomarker is Axl. In some embodiments an increase in the activity, expression, or amount of Axl in the sample profile as compared with a control profile after contacting the subject with a cancer therapeutic agent is indicative of higher susceptibility to treatment with an agent capable of inhibiting or reversing EMT or a chemotherapeutic agent.

Subject

A “subject” as referred to herein is any species of animal classified as a mammal, and includes but is not restricted to, domestic and farm animals, primates and humans. In some preferred embodiments the subject is a human of any sex or race. In some embodiments, the human is an adult human.

In some embodiments of this aspect of the disclosure, the subject was selected for treatment with the agent capable of inhibiting or reversing EMT or a chemotherapeutic agent, using a predictive method according to the first aspect of the disclosure outlined above.

Agents Capable of Inhibiting or Reversing EMT

The present disclosure provides methods for predicting a cancer-related outcome in a subject having, suspected of having, or diagnosed with acute myelocytic leukemia (AML). A “cancer-related outcome” is a clinical prediction or prognosis associated with cancer.

In some embodiments the cancer-related outcome is susceptibility to treatment with an agent capable of inhibiting or reversing EMT or a chemotherapeutic agent. The term “agent capable of inhibiting EMT” (used interchangeably with the term “EMT inhibitor”) as used herein means an agent that prevents or reduces the rate of the epithelial-mesenchymal transition (EMT). The term “agent capable of reversing EMT” as used herein means an agent which promotes the reverse of EMT, that is, promotes the mesenchymal-to-epithelial (MET) transition.

Agents capable of inhibiting or reversing EMT include inhibitors of the Axl kinase (Axl inhibitors) and inhibitors of the Akt3 kinase (Akt3 inhibitors). Accordingly, in some embodiments the agent capable of inhibiting or reversing EMT or chemotherapeutic agent is an Axl inhibitor, Akt3 inhibitor, Slfn11 inhibitor, or PHGDH inhibitor. In the most preferred embodiments the agent capable of inhibiting or reversing EMT or chemotherapeutic agent is an Axl inhibitor.

Axl inhibitors include, for example the small molecule Axl inhibitor BGB324/R428/bemcentinib, as well as cabozantinib, TP-0903, foretinib, merestinib, bosutinib, gilteritinib, crizotinib, amuvatinib, sunitinib, sitravatinib, LDC1267, and UNC2025. Other Axl inhibitors include the anti-Axl antibodies described in WO2015/193428, WO2015/193430, WO2016/097370, and WO2016/166296. In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In preferred embodiments, the agent capable of inhibiting or reversing EMT or chemotherapeutic agent is an Akt3 inhibitor. Akt3 inhibitors are described, for example in WO2016/102672. Accordingly, in some embodiments, the agent capable of inhibiting or reversing EMT or chemotherapeutic agent is an Akt3 inhibitor described in WO2016/102672.

In some embodiments the agent capable of inhibiting or reversing EMT is not an Akt3 inhibitor. In some embodiments the agent capable of inhibiting or reversing EMT is not a Slfn11 inhibitor. In some embodiments the agent capable of inhibiting or reversing EMT is not a PHGDH inhibitor.

In some embodiments of the predictive methods described herein, the agent capable of inhibiting or reversing EMT is administered as a single agent. In some other embodiments, the agent capable of inhibiting or reversing EMT is administered in combination with a further cancer treatment.

Suitable further cancer treatments include but are not limited to:
(i) alkylating agents, including alkyl sulfonates such as busulfan;
(ii) nitrogen mustards such as chlorambucil, cyclophosphamide, estramustine, ifosfamide, mechlorethamine, melphalan, and uramustine;
(iii) ethyleneimine derivatives such as thiotepa;
(iv) nitrosoureas such as carmustine, lomustine, and streptozocin;
(v) triazenes such as dacarbazine, procarbazine, and temozolamide;
(vi) platinum compounds such as cisplatin, carboplatin, oxaliplatin, satraplatin, and picoplatin onnaplatin, tetraplatin, sprioplatin, iproplatin, chloro(diethylenediamino)-platinum (II) chloride, dichloro(ethylenediamino)-platinum (II), diamino(2-ethylmalonato)platinum (II), (1,2-diaminocyclohexane)malonatoplatinum (II), (4-carboxyphthalo)-(1,2-diaminocyclohexane)platinum (II), (1,2-diaminocyclohexane)-(isocitrato)platinum (II), and (1,2-diaminocyclohexane)-cis-(pyruvato)platinum (II);
(vii) antimetabolites, including antifolates such as methotrexate, permetrexed, raltitrexed, and trimetrexate;
(viii) pyrimidine analogues such as azacitidine, capecitabine, cytarabine, edatrexate, floxuridine, fluorouracil, gemcitabine, and troxacitabine;
(ix) purine analogues such as cladribine, chlorodeoxyadenosine, clofarabine, fludarabine, mercaptopurine, pentostatin, and thioguanine;
(x) natural products, including antitumor antibiotics such as bleomycin, dactinomycin, mithramycin, mitomycin, mitoxantrone, porfiromycin;
(xi) anthracyclines such as daunorubicin, doxorubicin, epirubicin, idarubicin, and valrubicin;
(xii) mitotic inhibitors such as the vinca alkaloids vinblastine, vinvesir, vincristine, vindesine, and vinorelbine;
(xiii) enzymes such as L-asparaginase and PEG-L-asparaginase;
(xiv) microtubule polymer stabilizers such as the taxanes paclitaxel and docetaxel;
(xv) topoisomerase I inhibitors such as the camptothecins irinotecan and topotecan;
(xvi) topoisomerase II inhibitors such as podophyllotoxin, amsacrine, etoposide, teniposide, losoxantrone and actinomycin;
(xvii) hormones and hormone antagonists, including
   androgens such as fluoxymesterone and testolactone;
   antiandrogens such as bicalutamide, cyproterone, flutamide, and nilutamide;
   corticosteroids such as dexamethasone and prednisone;
   aromatase inhibitors such as aminoglutethimide, anastrozole, exemestane, formestane, and letrozole;
   estrogens such as diethylstilbestrol;
   antiestrogens such as fulvestrant, raloxifene, tamoxifen, and toremifine;
   luteinising hormone-releasing hormone (LHRH) agonists and antagonists such as abarelix, buserelin, goserelin, leuprolide, histrelin, desorelin, nafarelin acetate and triptorelin;
   progestins such as medroxyprogesterone acetate and megestrol acetate; and
   thyroid hormones such as levothyroxine and liothyronine;
(xviii) PKB pathway inhibitors, including perifosine, enzastaurin hydrochloride, and triciribine;
(xix) P13K inhibitors such as semaphore and SF1126, and MTOR inhibitors such as rapamycin and analogues;
(xx) CDK inhibitors, including seliciclib, alvocidib, and 7-hydroxystaurosporine;
(xxi) COX-2 inhibitors, including celecoxib;
(xxii) HDAC inhibitiors, including trichostatin A, suberoylanilide hydroxamic acid, and chlamydocin;
(xxiii) DNA methylase inhibitors, including temozolomide, and miscellaneous agents, including altretamine, arsenic trioxide, thalidomide, lenalidomide, gallium nitrate, levamisole, mitotane, hydroxyurea, octreotide, procarbazine, suramin, photodynamic compounds such as methoxsalen and sodium porfimer;
(xxiv) proteasome inhibitors such as bortezomib;
(xxv) molecular targeted therapy agents including: functional therapeutic agents, including gene therapy agents and antisense therapy agents;
(xxvi) tyrosine kinase inhibitors such as erlotinib hydrochloride, gefitinib, imatinib mesylate, and semaxanib;
(xxvii) Raf inhibitors such as sorafenib, and gene expression modulators such as the retinoids and rexinoids, for example adapalene, bexarotene, trans-retinoic acid, 9-cis-retinoic acid, and N-(4-hydroxyphenyl)retinamide;
(xxviii) phenotype-directed therapy agents, including monoclonal antibodies such as pembrolizumab, alemtuzumab, bevacizumab, cetuximab, ibritumomab tiuxetan, rituximab, and trastuzumab, immunotoxins such as gemtuzumab ozogamicin, radioimmunoconjugates such as I-tositumobab;
(xxix) cancer vaccines;
(xxx) biologic therapy agents including: interferons such as interferon-[alpha]2a and interferon-[alpha]2b, and interleukins such as aldesleukin, denileukin diftitox, and oprelvekin;
(xxxi) anticancer therapies involving the use of protective or adjunctive agents, including: cytoprotective agents such as amifostine, and dexrazoxane, phosphonates such as pamidronate and zoledronic acid, and stimulating factors such as epoetin, darbeopetin, filgrastim, PEG-filgrastim, and sargramostim;
(xxxii) Axl inhibitors such as 1-(6,7-dihydro-5H-benzo[6,7]cyclohepta[1,2-c]pyridazin-3-yl)-N3-((7-(S)-pyrrolidin-1-yl)-6,7,8,9-tetrahydro-5H-benzo[7]annulene-2-yl)-1H-1,2,4-triazole-3,5-diamine; and/or
(xxxiii) further combination chemotherapeutic regimens, such as combinations of carboplatin/paclitaxel, capecitabine/docetaxel, fluorauracil/levamisole, fluorauracil/leucovorin, methotrexate/leucovorin, and trastuzumab/paclitaxel, alone or in further combination with carboplatin.

In some preferred embodiments the further cancer treatment is a pyrimidine analogue, for example azacitidine, capecitabine, cytarabine, edatrexate, floxuridine, fluorouracil, gemcitabine, or troxacitabine, or a cytidine analogue, for example decitabine. In some particularly preferred embodiments the further cancer treatment is cytarabine. In other particularly preferred embodiments the further cancer treatment is decitabine.

Assessing Biomarker Activity, Expression, Amount

In some embodiments of the predictive methods described herein, assessing the activity, expression, or amount of one or more biomarker in a subject, or in a sample derived from a subject, comprises: contacting a sample, or an extract from a sample, with at least 1, 2, 3, 4, 5, 6, 7, 8, 9, 10 or more reagents for detecting the activity, expression, or amount of one or more biomarkers, each of which is selective for a particular biomarker; and, detecting and/or quantifying said reagents for detecting.

In some preferred embodiments, assessing the expression or amount of the one or more biomarker comprises determining the level of the one or more biomarker protein or mRNA in the subject or sample derived from the subject. Preferably the expression or amount of the one or more biomarker is assessed by determining the level of protein expression. In particularly preferred embodiments the expression or amount of the one or more biomarker is assessed by determining the level of protein expression in serum.

The term “expression” refers to the transcription of a gene's DNA template to produce the corresponding mRNA and translation of this mRNA to produce the corresponding gene product (i.e., a peptide, polypeptide, or protein) as well as the “expression” of a protein in one or more forms that may have been modified post translation.

In some embodiments determining the level of the one or more biomarker protein or mRNA comprises: contacting a sample, or an extract from a sample, with at least 1, 2, 3, 4, 5, 6, 7, 8, 9, 10 or more specific binding members, each of which selectively binds to a respective biomarker protein or mRNA; and, detecting and/or quantifying formation of complex formed by said specific binding member and said biomarker protein or mRNA. In some embodiments the specific binding member may comprise an antibody molecule or a binding fragment thereof.

A range of suitable techniques for determining protein and mRNA expression levels are well known in the art, for example microarray analysis, Western blotting, and PCR techniques such as QPCR. In some embodiments, determining the expression or amount of the one or more biomarker protein or mRNA may comprise use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.

In some embodiments, the expression or amount of the one or more biomarker is determined as an absolute amount of the respective biomarker protein or mRNA in the sample. In such cases, the respective biomarker protein or mRNA may be quantified in the sample and the amount expressed as a measure of the sample, for example the volume, mass, or total protein content of the sample.

In some embodiments, the expression or amount of the one or more biomarker is determined as a relative amount of the respective biomarker protein or mRNA in the sample, wherein the relative amount is determined relative to a reference protein or mRNA in the sample. Determining the relative amount of biomarker protein or mRNA in this way allows for normalisation to, e.g., account for differences in total protein concentration and to remove bias from sample to sample.

Preferably the reference protein or mRNA is one whose expression or amount does not vary significantly between subjects having cancer and subjects not having cancer, in particular AML. That is, the reference protein or mRNA is preferably one whose expression or amount is not altered by AML. Accordingly, the reference protein or mRNA may comprise one or more protein or mRNA which is not one of the biomarkers described herein. When expressing the expression or amount of the one or more biomarker as a relative amount, the absolute amount of the respective biomarker protein or mRNA in the sample may be divided by the absolute amount of the reference protein or mRNA in the sample.

Assessing Biomarker Expression at the RNA Level

Gene expression can be detected at the RNA level. RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland). Typical assay formats utilising ribonucleic acid hybridisation include nuclear run-on assays, RT-PCR, RNase protection assays (Melton et al., Nuc. Acids Res. 12:7035), Northern blotting and In Situ hybridization. Gene expression can also be detected by microarray analysis as described below.

Assessing Biomarker Expression at the Protein Level

Preferably biomarker level is assessed by measuring protein expression. Altered gene or protein expression may also be detected by measuring the polypeptides encoded by the gene. This may be achieved by using molecules which bind to the polypeptides encoded by the biomarker gene. Suitable molecules/agents which bind either directly or indirectly to the polypeptides in order to detect the presence of the protein include naturally occurring molecules such as peptides and proteins, for example antibodies, or they may be synthetic molecules.

Antibodies may be derived from commercial sources or through techniques which are familiar to those skilled in the art. In one embodiment, and where altered expression manifests itself through the expression of alteration of post translationally-modified forms of a protein biomarker, antibodies specific for those different forms may be used. For the purposes of this disclosure, the term “antibody”, unless specified to the contrary, includes whole antibodies, or fragments of whole antibodies which retain their binding activity for a target antigen. Such fragments include Fv, F(ab′) and F(ab′)2 fragments, as well as single chain antibodies (scFv). Furthermore, the antibodies and fragments thereof may be humanised antibodies, for example as described in EP239400A. For example: monoclonal and polyclonal antibodies, recombinant antibodies, proteolytic and recombinant fragments of antibodies (Fab, Fv, scFv, diabodies), single-domain antibodies (VHH, sdAb, nanobodies, IgNAR, VNAR), and proteins unrelated to antibodies, which have been engineered to have antibody-like specific binding. Antibodies may be bound to a solid support and/or packaged into kits in a suitable container along with suitable reagents, controls, instructions and the like.

Arrays

Array technology and the various techniques and applications associated with it are well known in the art. Array technology overcomes the disadvantages with traditional methods in molecular biology, which generally work on a “one gene in one experiment” basis, resulting in low throughput and the inability to appreciate the “whole picture” of gene function. In the context of the products and methods of the present disclosure, array technology can be used, for example, in the analysis of the expression of biomarker proteins or mRNA.

In general, any library or group of samples may be arranged in an orderly manner into an array, by spatially separating the members of the library or group. Examples of suitable libraries for arraying include nucleic acid libraries (including DNA, cDNA, oligonucleotide, etc. libraries), peptide, polypeptide and protein libraries, as well as libraries comprising any molecules, such as ligand libraries, among others. Accordingly, where reference is made to a “library” in this document, unless the context dictates otherwise, such reference should be taken to include reference to a library in the form of an array.

Proteins, polypeptides, etc., may also be immobilised in arrays. For example, antibodies have been used in microarray analysis of the proteome using protein chips (Borrebaeck C A, 2000, Immunol Today 21(8):379-82). Polypeptide arrays are reviewed in, for example, MacBeath and Schreiber, 2000, Science, 289(5485):1760-1763.

Sample

Suitable samples include, but are not limited to, tissue samples such as tissue biopsy, blood, urine, buccal scrapes etc., as well as serum, plasma, or tissue culture supernatant samples. In preferred embodiments the expression or amount of the one or more biomarker is assessed by determining the level of protein expression in the sample. In some preferred embodiments the sample is a blood, serum, or plasma sample. In some particularly preferred embodiments the sample is a serum sample.

When assessing the expression or amount of the one or more biomarker proteins or mRNA in serum or plasma samples derived from a subject, samples are removed and subjected to analytical techniques such as flow cytometry, mass cytometry (CyTOF), ELISA, PET, and SELDI-TOF MS. In some embodiments, the method may comprise extracting RNA from said sample and detecting gene expression by QPCR. In some other embodiments, gene expression may be detected by detecting protein products by, for example, Western Blot.

In some embodiments, the level of expression of the one or more biomarker is assessed by determining the copy number of the gene encoding the one or more biomarker. Copy number (i.e. gene duplication events) may be determined using standard techniques known in the art, e.g. using a DNA chip as described in Jiang et al. (Jiang Q, Ho Y Y, Hao L, Nichols Berrios C, Chakravarti A. Copy number variants in candidate genes are genetic modifiers of Hirschsprung disease. PLoS One. 2011; 6(6)).

Is some preferred embodiments of the predictive methods described herein, the method is performed in vitro or ex vivo.

Methods of Selecting a Subject for Treatment

A third aspect of this facet of the present disclosure relates to methods of selecting subjects having, suspected of having, or diagnosed with acute myelocytic leukemia (AML) for treatment with an agent capable of inhibiting or reversing EMT or a chemotherapeutic agent. In some embodiments the method comprises: identifying subjects susceptible to treatment with an agent capable of inhibiting or reversing EMT or a chemotherapeutic agent using a predictive method defined in accordance with the above described first and second aspects of the disclosure; and, selecting thus identified subjects for treatment.

In some embodiments this aspect relates to methods of selecting subjects having, suspected of having, or diagnosed with acute myelocytic leukemia (AML) for continued treatment with an agent capable of inhibiting or reversing EMT or a chemotherapeutic agent. In some embodiments the method comprises: identifying subjects susceptible to treatment with an agent capable of inhibiting or reversing EMT or a chemotherapeutic agent using a predictive method defined in accordance with the above described second aspect of the disclosure; and, selecting thus identified subjects for treatment.

In some embodiments of this aspect of the disclosure, the treatment comprises administering to the subject a therapeutically effective amount of an agent capable of inhibiting or reversing EMT or a chemotherapeutic agent as described herein. Accordingly, in some embodiments the treatment comprises an Axl inhibitor, Akt3 inhibitor, Slfn11 inhibitor, or PHGDH inhibitor. In some preferred embodiments the treatment comprises an Axl inhibitor, for example, BGB324/R428/bemcentinib, cabozantinib, TP-0903, foretinib, merestinib, bosutinib, gilteritinib, crizotinib, amuvatinib, sunitinib, sitravatinib, LDC1267, or UNC2025. Other Axl inhibitors include the anti-Axl antibodies described in WO2015/193428, WO2015/193430, WO2016/097370, and WO2016/166296. In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In some other preferred embodiments, the treatment comprises an Akt3 inhibitor. In some embodiments the Akt3 inhibitor is an Akt3 inhibitor disclosed in WO2016/102672.

In some embodiments of this aspect of the disclosure, the treatment is administered as a single agent. In other embodiments, the treatment is administered in combination with a further cancer treatment. Suitable further cancer treatments are outlined in detail above. In some embodiments the further cancer treatment is a pyrimidine analogue, for example azacitidine, capecitabine, cytarabine, edatrexate, floxuridine, fluorouracil, gemcitabine, or troxacitabine, or a cytidine analogue, for example decitabine. In some particularly preferred embodiments the further cancer treatment is cytarabine. In other particularly preferred embodiments the further cancer treatment is decitabine.

Kits

A fourth aspect of this facet of the present disclosure relates to diagnostic kits and test devices. In some embodiments the disclosure provides a diagnostic kit comprising 1, 2, 3, 4, 5, 6, 7, 8, 9, 10 or more reagents for detecting the activity, expression, or amount of one or more of the biomarkers described herein, each of which is selective for a particular biomarker. In some embodiments each of the reagents for detecting is selective for a biomarker selected from the group consisting of: Axl, FGF-21, RAGE, CEA, FSH, MMP-10, Omentin, CA-19-9, LH, Haptoglobin, NAP-2, IgE, and/or CD40-L.

In other embodiments the disclosure provides a test device comprising 1, 2, 3, 4, 5, 6, 7, 8, 9, 10 or more reagents for detecting the activity, expression, or amount of one or more of the biomarkers described herein, each of which is selective for a particular biomarker. In some embodiments each of the reagents for detecting is selective for a biomarker selected from the group consisting of: Axl, FGF-21, RAGE, CEA, FSH, MMP-10, Omentin, CA-19-9, LH, Haptoglobin, NAP-2, IgE, and/or CD40-L.

In some embodiments the diagnostic kit or test device comprises 1, 2, 3, 4, 5, 6, 7, 8, 9, 10 or more specific binding members, each of which selectively binds to one of the biomarkers described herein; and, one or more reagents for detecting said one or more specific binding members, or one or more reagents for detecting and/or quantifying formation of a complex formed by said specific binding member and said biomarker. In some embodiments each specific binding member selectively binds to a biomarker selected from the group consisting of: Axl, FGF-21, RAGE, CEA, FSH, MMP-10, Omentin, CA-19-9, LH, Haptoglobin, NAP-2, IgE, and/or CD40-L.

In some embodiments the diagnostic kit or test device comprises a specific binding member which selectively binds to Axl, and at least 1, 2, 3, 4, 5, 6, 7, 8, 9, 10, or more specific binding members, each of which selectively binds to a biomarker selected from the group consisting of: FGF-21, RAGE, CEA, FSH, MMP-10, Omentin, CA-19-9, LH, Haptoglobin, NAP-2, IgE, and/or CD40-L.

In some embodiments of this aspect of the disclosure the diagnostic kit or test device comprises a plurality of said specific binding members provided in the form of an array on a substrate or conjugated to a plurality of particles, such as beads or microspheres. The particles may be encoded with a detectable label. In some embodiments the specific binding member comprises an antibody molecule or a binding fragment thereof.

In some embodiments of the diagnostic kit or test device detecting formation of a complex formed by a specific binding member and a biomarker is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.

In some embodiments of the diagnostic kit or test device, the kit or test device comprises no more than 15, 20, 25, 30, 40, or 50 reagents for detecting. In some embodiments of the diagnostic kit or test device, the kit or test device comprises no more than 15, 20, 25, 30, 40, or 50 specific binding members.

The present disclosure also provides use of such diagnostic kits and test devices in methods of predicting a cancer-related outcome in a subject. In preferred embodiments the cancer-related outcome is susceptibility to treatment with an agent capable of inhibiting or reversing EMT or a chemotherapeutic agent as described herein.

In some embodiments the agent capable of inhibiting or reversing EMT or chemotherapeutic agent is an Axl inhibitor, Akt3 inhibitor, Slfn11 inhibitor, or PHGDH inhibitor. In some preferred embodiments the agent capable of inhibiting or reversing EMT or chemotherapeutic agent is an Axl inhibitor, for example BGB324/R428/bemcentinib, cabozantinib, TP-0903, foretinib, merestinib, bosutinib, gilteritinib, crizotinib, amuvatinib, sunitinib, sitravatinib, LDC1267, or UNC2025. Other Axl inhibitors include the anti-Axl antibodies described in WO2015/193428, WO2015/193430, WO2016/097370, and WO2016/166296. In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In some embodiments of the diagnostic kit or test device for use in a predictive method, the subject has, is suspected of having, or has been diagnosed with AML.

In some embodiments the method may be as defined in accordance with the above described first and second aspects of the disclosure.

A fifth aspect of this facet of the present disclosure relates to the use of a reagent for detecting the activity, expression, or amount of a biomarker selected from the group consisting of: Axl, FGF-21, RAGE, CEA, FSH, MMP-10, Omentin, CA-19-9, LH, Haptoglobin, NAP-2, IgE, and/or CD40-L, in a method of predicting a cancer-related outcome in a subject.

A sixth aspect of this facet of the disclosure relates to the use of 1, 2, 3, 4, 5, 6, 7, 8, 9, 10 or more reagents for detecting the activity, expression, or amount a biomarker, each of which is selective for a particular biomarker selected from the group consisting of: Axl, FGF-21, RAGE, CEA, FSH, MMP-10, Omentin, CA-19-9, LH, Haptoglobin, NAP-2, IgE, and/or CD40-L, in a method of manufacture of a diagnostic kit or test device for use in a method of predicting a cancer-related outcome in a subject. In some embodiments of these uses the reagent for detecting comprises a specific binding member selective for a particular biomarker. The specific binding member may comprise an antibody molecule or a binding fragment thereof.

In some embodiments the method may be as defined in accordance with the above described first and second aspects of this facet of the disclosure.

Uses

A seventh aspect of this facet of the present disclosure relates to the use of one or more of: Axl, FGF-21, RAGE, CEA, FSH, MMP-10, Omentin, CA-19-9, LH, Haptoglobin, NAP-2, IgE, and/or CD40-L, as a biomarker for determining if a subject is susceptible to treatment with an agent capable of inhibiting or reversing EMT or a chemotherapeutic agent.

In some embodiments the use relates to use of Axl, and at least 1, 2, 3, 4, 5, 6, 7, 8, 9, 10, or more of: FGF-21, RAGE, CEA, FSH, MMP-10, Omentin, CA-19-9, LH, Haptoglobin, NAP-2, IgE, and/or CD40-L, as a biomarker for determining if a subject is susceptible to treatment with an agent capable of inhibiting or reversing EMT or a chemotherapeutic agent.

In some embodiments the use is as a biomarker in a method as defined in accordance with the above described first and second aspects of this facet of the disclosure.

Methods of Treating a Subject

An eighth aspect of this facet of the present disclosure relates to methods of treating a subject having, suspected of having, or diagnosed with acute myelocytic leukemia (AML). In some embodiments the method comprises: identifying a subject for treatment using a predictive method defined in accordance with the above described first and second aspects of this facet of the disclosure; and, administering to the subject a therapeutically effective amount of an agent capable of inhibiting or reversing EMT or a chemotherapeutic agent.

Accordingly, in some embodiments, the method comprises: (a) obtaining a sample from the subject; (b) determining if the subject is susceptible to treatment with an agent capable of inhibiting or reversing EMT or a chemotherapeutic agent using a predictive method defined in accordance with the above described first and second aspects of this facet of the disclosure; and, (c) administering to the subject a therapeutically effective amount of an agent capable of inhibiting or reversing EMT or a chemotherapeutic agent.

In some embodiments the method comprises administering a therapeutically effective amount of an agent capable of inhibiting or reversing EMT or chemotherapeutic agent to a subject identified as susceptible to said treatment using a predictive method defined in accordance with the above described first and second aspects of this facet of the disclosure.

In other embodiments the method comprises: (a) administering to a subject a therapeutically effective amount of an agent capable of inhibiting or reversing EMT or a chemotherapeutic agent; and, (b) determining if the subject is susceptible to said treatment using a predictive method defined in accordance with the above described second aspect of this facet of the disclosure. In some embodiments the method further comprises: (c) administering to the subject one or more further therapeutically effective amounts of an agent capable of inhibiting or reversing EMT or a chemotherapeutic agent provided that the subject is identified as susceptible to said treatment.

A ninth aspect of this facet of the present disclosure relates to an agent capable of inhibiting or reversing EMT or chemotherapeutic agent for use in a method of treating a subject having, suspected of having, or diagnosed with acute myelocytic leukemia (AML). In some embodiments the method of treatment is a method as defined above.

A tenth aspect of this facet of the present disclosure relates to use of an agent capable of inhibiting or reversing EMT or chemotherapeutic agent for use in a method of manufacture of a medicament for use in a method of treating a subject having, suspected of having, or diagnosed with acute myelocytic leukemia (AML). In some embodiments the method of treatment is a method as defined above.

In some embodiments of these methods, agents for use, or uses, the agent capable of inhibiting or reversing EMT or chemotherapeutic agent comprises an Axl inhibitor, Akt3 inhibitor, Slfn11 inhibitor, or PHGDH inhibitor. In some preferred embodiments the agent capable of inhibiting or reversing EMT or chemotherapeutic agent is an Axl inhibitor, for example BGB324/R428/bemcentinib, cabozantinib, TP-0903, foretinib, merestinib, bosutinib, gilteritinib, crizotinib, amuvatinib, sunitinib, sitravatinib, LDC1267, or UNC2025. Other Axl inhibitors include the anti-Axl antibodies described in WO2015/193428, WO2015/193430, WO2016/097370, and WO2016/166296. In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In some other preferred embodiments, the treatment comprises an Akt3 inhibitor. In some embodiments the Akt3 inhibitor is an Akt3 inhibitor disclosed in WO2016/102672.

In some embodiments of these methods, agents for use, or uses, the treatment is administered as a single agent. In other embodiments, the treatment is administered in combination with a further cancer treatment. Suitable further cancer treatments are outlined in detail above. In some embodiments the further cancer treatment is a pyrimidine analogue, for example azacitidine, capecitabine, cytarabine, edatrexate, floxuridine, fluorouracil, gemcitabine, or troxacitabine, or a cytidine analogue, for example decitabine. In some particularly preferred embodiments the further cancer treatment is cytarabine. In other particularly preferred embodiments the further cancer treatment is decitabine.

Statements of Disclosure

The following numbered statements relate to aspects of this facet of the present disclosure, and form part of the description:
101. A method of predicting a cancer-related outcome in a subject, the method comprising assessing the activity, expression, or amount of one or more biomarker in the subject, or in a sample derived from the subject;
   wherein the one or more biomarker is selected from the group consisting of: Axl, FGF-21, RAGE, CEA, FSH, MMP-10, Omentin, CA-19-9, LH, Haptoglobin, NAP-2, IgE, and/or CD40-L; and
   wherein the subject has, is suspected of having, or has been diagnosed with acute myelocytic leukemia (AML).
102. A method according to statement 101, wherein the method comprises:
   assessing the activity, expression, or amount of one or more biomarker in the subject, or in a sample derived from the subject, to obtain a sample profile of the one or more biomarker; and
   making a prediction based on the sample profile of the one or more biomarker.
103. A method according to statement 102, wherein the prediction is made by comparing the sample profile to a control profile.
104. A method according to statement 103, wherein the sample profile is obtained before the subject is contacted with or administered an agent capable of inhibiting or reversing EMT or a chemotherapeutic agent.
105. A method according to statement 104, wherein the control profile is:
   (i) obtained from a population of control subjects having AML; or
   (ii) obtained from a control subject or population of control subjects having AML and previously found to lack susceptibility to treatment with an agent capable of inhibiting or reversing EMT or a chemotherapeutic agent (that is, non-responder subjects);
   (iii) a predetermined profile of biomarker expression, activity, or amount values, for example a profile of “average, median, or mean” values or “standard ranges” of values, obtained from a control subject or population of control subjects having AML and previously found to lack susceptibility to treatment with an agent capable of inhibiting or reversing EMT or a chemotherapeutic agent (that is, non-responder subjects);
   (iv) obtained from a control sample having a known “average, median, or mean” value of biomarkers indicative of a control subject or population of control subjects having AML and previously found to lack susceptibility to treatment with an agent capable of inhibiting or reversing EMT or a chemotherapeutic agent (that is, non-responder subjects);
   (v) a predetermined profile of biomarker expression, activity, or amount “threshold” values obtained from a control subject or population of control subjects having AML and previously found to be susceptible to treatment with an agent capable of inhibiting or reversing EMT or a chemotherapeutic agent (that is, responder subjects); or
   (vi) obtained from a control sample having a known “threshold” value of biomarkers indicative of a control subject or population of control subjects having AML and previously found to be susceptible to treatment with an agent capable of inhibiting or reversing EMT or a chemotherapeutic agent (that is, responder subjects).
106. A method according to statement 105, wherein a higher activity, expression, or amount of one or more biomarker in the sample profile as compared with the control profile is indicative of susceptibility to treatment with an agent capable of inhibiting or reversing EMT or a chemotherapeutic agent.
107. A method according to statement 105, wherein a lower activity, expression, or amount of one or more biomarker in the sample profile as compared with the control profile is indicative of susceptibility to treatment with an agent capable of inhibiting or reversing EMT or a chemotherapeutic agent.
108. A method according to any one of statements 105-107, wherein the one or more biomarker includes at least one of: Haptoglobin, NAP-2, IgE, and/or CD40-L,
   and wherein a higher activity, expression, or amount of one or more of said biomarkers in the sample profile as compared with the control profile is indicative of susceptibility to treatment with an agent capable of inhibiting or reversing EMT or a chemotherapeutic agent.
109. A method according to any one of statements 105-108, wherein the one or more biomarker includes at least one of: Axl, FGF-21, RAGE, CEA, FSH, MMP-10, Omentin, CA-19-9, and/or LH,
   and wherein a lower activity, expression, or amount of one or more of said biomarkers in the sample profile as compared with the control profile is indicative of susceptibility to treatment with an agent capable of inhibiting or reversing EMT or a chemotherapeutic agent.
110. A method according to any one of statements 105-109, wherein:
   a lower activity, expression, or amount of Axl; and/or
   a lower activity, expression, or amount of FGF-21; and/or
   a lower activity, expression, or amount of RAGE; and/or
   a lower activity, expression, or amount of CEA; and/or
   a lower activity, expression, or amount of FSH; and/or
   a lower activity, expression, or amount of MMP-10; and/or
   a lower activity, expression, or amount of Omentin; and/or
   a lower activity, expression, or amount of CA-19-9; and/or
   a lower activity, expression, or amount of LH; and/or
   a higher activity, expression, or amount of Haptoglobin; and/or
   a higher activity, expression, or amount of NAP-2; and/or
   a higher activity, expression, or amount of IgE; and/or
   a higher activity, expression, or amount of CD40-L;
   in the sample profile as compared with the control profile is indicative of susceptibility to treatment with an agent capable of inhibiting or reversing EMT or a chemotherapeutic agent.
111. A method according to any one of statements 101-110, wherein the one or more biomarker comprises Axl,
   optionally wherein the one or more biomarker comprises Axl and at least 1, 2, 3, 4, 5, 6, 7, 8, 9, 10, or more further biomarkers selected from: FGF-21, RAGE, CEA, FSH, MMP-10, Omentin, CA-19-9, LH, Haptoglobin, NAP-2, IgE, and/or CD40-L.
112. A method according to any one of statements 101-111, wherein:
   said Axl biomarker comprises an amino acid sequence having at least 70%, 80%, 90%, 95%, 99% or 100% sequence identity with the full-length of the amino acid sequence disclosed at UniProt accession no: P30530-1 or P30530-2 (entry version 196) or a fragment thereof;
   said Fibroblast Growth Factor 21 (FGF-21) biomarker comprises an amino acid sequence having at least 70%, 80%, 90%, 95%, 99% or 100% sequence identity with the full-length of the amino acid sequence disclosed at UniProt accession no: Q9NSA1 (entry version 145) or a fragment thereof;
   said Receptor for advanced glycosylation end products (RAGE) biomarker comprises an amino acid sequence having at least 70%, 80%, 90%, 95%, 99% or 100% sequence identity with the full-length of the amino acid sequence disclosed at UniProt accession no: Q15109-1, Q15109-2, Q15109-3, Q15109-4, Q15109-5, Q15109-6, Q15109-7, Q15109-8, Q15109-9, Q15109-10 (entry version 189) or a fragment thereof;
   said Carcinoembryonic Antigen (CEA) biomarker comprises an amino acid sequence having at least 70%, 80%, 90%, 95%, 99% or 100% sequence identity with the full-length of the amino acid sequence disclosed at UniProt accession no: P06731-1 or P06731-2 (entry version 179) or a fragment thereof;
   said Follicle-Stimulating Hormone (FSH) biomarker comprises an amino acid sequence having at least 70%, 80%, 90%, 95%, 99% or 100% sequence identity with the full-length of the amino acid sequence disclosed at UniProt accession no: P01225 (entry version 189) or a fragment thereof;
   said Matrix Metalloproteinase-10 (MMP-10) biomarker comprises an amino acid sequence having at least 70%, 80%, 90%, 95%, 99% or 100% sequence identity with the full-length of the amino acid sequence disclosed at UniProt accession no: P09238 (entry version 185) or a fragment thereof;
   said Omentin biomarker comprises an amino acid sequence having at least 70%, 80%, 90%, 95%, 99% or 100% sequence identity with the full-length of the amino acid sequence disclosed at UniProt accession no: Q8WWA0 (entry version 127) or a fragment thereof;
   said Cancer Antigen 19-9 (CA-19-9) biomarker comprises an amino acid sequence having at least 70%, 80%, 90%, 95%, 99% or 100% sequence identity with the full-length of the amino acid sequence disclosed at UniProt accession no: Q9BXJ9-1 or Q9BXJ9-2 (entry version 160) or a fragment thereof;
   said Luteinizing Hormone (LH) biomarker comprises an amino acid sequence having at least 70%, 80%, 90%, 95%, 99% or 100% sequence identity with the full-length of the amino acid sequence disclosed at UniProt accession no: P01229 (entry version 179) or a fragment thereof;
   said Haptoglobin (HP) biomarker comprises an amino acid sequence having at least 70%, 80%, 90%, 95%, 99% or 100% sequence identity with the full-length of the amino acid sequence disclosed at UniProt accession no: P00738-1 or P00738-2 (entry version 200) or a fragment thereof;
   said Neutrophil Activating Peptide 2 (NAP-2) biomarker comprises an amino acid sequence having at least 70%, 80%, 90%, 95%, 99% or 100% sequence identity with the full-length of the amino acid sequence disclosed at UniProt accession no: P02775 (entry version 191) or a fragment thereof;
   said Immunoglobulin E (IgE) biomarker comprises an amino acid sequence having at least 70%, 80%, 90%, 95%, 99% or 100% sequence identity with the full-length of the amino acid sequence disclosed at UniProt accession no: P01854 (entry version 162) or a fragment thereof; and/or
   said CD40 Ligand (CD40-L) biomarker comprises an amino acid sequence having at least 70%, 80%, 90%, 95%, 99% or 100% sequence identity with the full-length of the amino acid sequence disclosed at UniProt accession no: P29965 (entry version 199) or a fragment thereof;
   or a nucleic acid (either DNA or RNA) having at least 70%, 80%, 90%, 95%, 99% or 100% sequence identity with the full-length nucleic acid sequence encoding said amino acid sequences.
113. A method according to any one of statement 101-112, wherein the method comprises determining the amount of at least 2, 3, 4, 5, 6, 7, 8, 9, 10, 11, 12, 13, 14, 15, 20 of said biomarkers in the subject, or sample derived from the subject.
114. A method according to any one of statements 104-113, wherein assessing the activity, expression, or amount of one or more biomarker in the subject, or in a sample derived from the subject, comprises:
   contacting a sample, or an extract from the sample, with at least 1, 2, 3, 4, 5, 6, 7, 8, 9, 10 or more reagents for detecting the activity, expression, or amount of one or more biomarkers, each of which is selective for a particular biomarker; and
   detecting and/or quantifying said reagents for detecting.
115. A method according to statement 114, wherein assessing the expression or amount of one or more biomarker in the subject, or in a sample derived from the subject, comprises:
   contacting a sample, or an extract from the sample, with at least 1, 2, 3, 4, 5, 6, 7, 8, 9, 10 or more specific binding members, each of which selectively binds to a respective biomarker protein or mRNA; and
   detecting and/or quantifying formation of complex formed by said specific binding member and said biomarker protein or mRNA.
116. A method according to statement 115 wherein the specific binding member comprises an antibody molecule or binding fragment thereof.
117. A method according to statement 115 or 116, wherein detecting formation of a complex formed by the specific binding member and the biomarker protein or mRNA is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.
118. A method according to any one of statements 115-117 wherein the expression or amount of the one or more biomarker is determined as the absolute amount of the respective biomarker protein or mRNA in the sample.
119. A method according to any one of statements 115-117 wherein the expression or amount of the one or more biomarker is determined as a relative amount of the respective biomarker protein or mRNA in the sample,
   optionally wherein the relative amount is determined relative to a reference protein or mRNA in the sample, the expression of which is not altered by AML.
120. A method according to any one of statements 101-119, wherein the cancer-related outcome is susceptibility to treatment with an agent capable of inhibiting or reversing EMT or a chemotherapeutic agent.
121. A method according to statement 120, wherein the agent capable of inhibiting or reversing EMT or chemotherapeutic agent is an Axl inhibitor, Akt3 inhibitor, Slfn11 inhibitor, or PHGDH inhibitor.
122. A method according to statement 121, wherein the agent capable of inhibiting or reversing EMT or chemotherapeutic agent is an Axl inhibitor.
123. A method according to statement 122, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
124. A method according to statement 123, wherein the Axl inhibitor is BGB324/R428/bemcentinib.
125. A method according to statement 123, wherein the Axl inhibitor is an anti-Axl antibody described in WO2015/193428, WO2015/193430, WO2016/097370, or WO2016/166296.
126. A method according to statement 121, wherein the agent capable of inhibiting or reversing EMT or chemotherapeutic agent is an Akt3 inhibitor.
127. A method according to statement 126, wherein the Akt3 inhibitor is an Akt3 inhibitor disclosed in WO2016/102672.
128. A method according to any one of statements 120-127, wherein the agent capable of inhibiting or reversing EMT is administered as a single agent.
129. A method according to any one of statements 120-127, wherein the agent capable of inhibiting or reversing EMT is administered in combination with a further cancer treatment.
130. A method according to statement 129, wherein the further cancer treatment is selected from:
   (i) alkylating agents, including alkyl sulfonates such as busulfan;
   (ii) nitrogen mustards such as chlorambucil, cyclophosphamide, estramustine, ifosfamide, mechlorethamine, melphalan, and uramustine;
   (iii) ethyleneimine derivatives such as thiotepa;
   (iv) nitrosoureas such as carmustine, lomustine, and streptozocin;
   (v) triazenes such as dacarbazine, procarbazine, and temozolamide;
   (vi) platinum compounds such as cisplatin, carboplatin, oxaliplatin, satraplatin, and picoplatin onnaplatin, tetraplatin, sprioplatin, iproplatin, chloro(diethylenediamino)-platinum (II) chloride, dichloro(ethylenediamino)-platinum (II), diamino(2-ethylmalonato)platinum (II), (1,2-diaminocyclohexane)malonatoplatinum (II), (4-carboxyphthalo)-(1,2-diaminocyclohexane)platinum (II), (1,2-diaminocyclohexane)-(isocitrato)platinum (II), and (1,2-diaminocyclohexane)-cis-(pyruvato)platinum (II);
   (vii) antimetabolites, including antifolates such as methotrexate, permetrexed, raltitrexed, and trimetrexate;
   (viii) pyrimidine analogues such as azacitidine, capecitabine, cytarabine, edatrexate, floxuridine, fluorouracil, gemcitabine, and troxacitabine;
   (ix) purine analogues such as cladribine, chlorodeoxyadenosine, clofarabine, fludarabine, mercaptopurine, pentostatin, and thioguanine;
   (x) natural products, including antitumor antibiotics such as bleomycin, dactinomycin, mithramycin, mitomycin, mitoxantrone, porfiromycin;
   (xi) anthracyclines such as daunorubicin, doxorubicin, epirubicin, idarubicin, and valrubicin;
   (xii) mitotic inhibitors such as the vinca alkaloids vinblastine, vinvesir, vincristine, vindesine, and vinorelbine;
   (xiii) enzymes such as L-asparaginase and PEG-L-asparaginase;
   (xiv) microtubule polymer stabilizers such as the taxanes paclitaxel and docetaxel;
   (xv) topoisomerase I inhibitors such as the camptothecins irinotecan and topotecan;
   (xvi) topoisomerase II inhibitors such as podophyllotoxin, amsacrine, etoposide, teniposide, losoxantrone and actinomycin;
   (xvii) hormones and hormone antagonists, including
      androgens such as fluoxymesterone and testolactone;
      antiandrogens such as bicalutamide, cyproterone, flutamide, and nilutamide;
      corticosteroids such as dexamethasone and prednisone;
      aromatase inhibitors such as aminoglutethimide, anastrozole, exemestane, formestane, and letrozole;
      estrogens such as diethylstilbestrol;
      antiestrogens such as fulvestrant, raloxifene, tamoxifen, and toremifine;
      luteinising hormone-releasing hormone (LHRH) agonists and antagonists such as abarelix, buserelin, goserelin, leuprolide, histrelin, desorelin, nafarelin acetate and triptorelin;
      progestins such as medroxyprogesterone acetate and megestrol acetate; and
      thyroid hormones such as levothyroxine and liothyronine;
   (xviii) PKB pathway inhibitors, including perifosine, enzastaurin hydrochloride, and triciribine;
   (xix) P13K inhibitors such as semaphore and SF1126, and MTOR inhibitors such as rapamycin and analogues;
   (xx) CDK inhibitors, including seliciclib, alvocidib, and 7-hydroxystaurosporine;
   (xxi) COX-2 inhibitors, including celecoxib;
   (xxii) HDAC inhibitiors, including trichostatin A, suberoylanilide hydroxamic acid, and chlamydocin;
   (xxiii) DNA methylase inhibitors, including temozolomide, and miscellaneous agents, including altretamine, arsenic trioxide, thalidomide, lenalidomide, gallium nitrate, levamisole, mitotane, hydroxyurea, octreotide, procarbazine, suramin, photodynamic compounds such as methoxsalen and sodium porfimer;
   (xxiv) proteasome inhibitors such as bortezomib;
   (xxv) molecular targeted therapy agents including: functional therapeutic agents, including gene therapy agents and antisense therapy agents;
   (xxvi) tyrosine kinase inhibitors such as erlotinib hydrochloride, gefitinib, imatinib mesylate, and semaxanib;
   (xxvii) Raf inhibitors such as sorafenib, and gene expression modulators such as the retinoids and rexinoids, for example adapalene, bexarotene, trans-retinoic acid, 9-cis-retinoic acid, and N-(4-hydroxyphenyl)retinamide;
   (xxviii) phenotype-directed therapy agents, including monoclonal antibodies such as pembrolizumab, alemtuzumab, bevacizumab, cetuximab, ibritumomab tiuxetan, rituximab, and trastuzumab, immunotoxins such as gemtuzumab ozogamicin, radioimmunoconjugates such as I-tositumobab;
   (xxix) cancer vaccines;
   (xxx) biologic therapy agents including: interferons such as interferon-[alpha]2a and interferon-[alpha]2b, and interleukins such as aldesleukin, denileukin diftitox, and oprelvekin; (xxxi) anticancer therapies involving the use of protective or adjunctive agents, including: cytoprotective agents such as amifostine, and dexrazoxane, phosphonates such as pamidronate and zoledronic acid, and stimulating factors such as epoetin, darbeopetin, filgrastim, PEG-filgrastim, and sargramostim;
   (xxxii) Axl inhibitors such as 1-(6,7-dihydro-5H-benzo[6,7]cyclohepta[1,2-c]pyridazin-3-yl)-N3-((7-(S)-pyrrolidin-1-yl)-6,7,8,9-tetrahydro-5H-benzo[7]annulene-2-yl)-1H-1,2,4-triazole-3,5-diamine; and/or
   (xxxiii) further combination chemotherapeutic regimens, such as combinations of carboplatin/paclitaxel, capecitabine/docetaxel, fluorauracil/levamisole, fluorauracil/leucovorin, methotrexate/leucovorin, and trastuzumab/paclitaxel, alone or in further combination with carboplatin.
131. A method according to statement 129, wherein the further cancer treatment is a pyrimidine analogue, for example azacitidine, capecitabine, cytarabine, edatrexate, floxuridine, fluorouracil, gemcitabine, or troxacitabine, or a cytidine analogue, for example decitabine.
132. A method according to statement 131, wherein the further cancer treatment is cytarabine.
133. A method according to statement 131, wherein the further cancer treatment is decitabine.
134. A method according to any one of statements 101-133, wherein the subject is mammalian.
135. A method according to statement 134, wherein the subject is human.
136. A method according to any one of statements 101-135, wherein the predictive method is performed before the subject receives a treatment or course of treatment for AML.
137. A method according to any one of statement 101-136, wherein the predictive method is performed during or after a treatment or course of treatment for AML.
138. A method according to any one of statements 101-135 or 137, wherein the subject has previously been treated with an agent capable of inhibiting or reversing EMT or a chemotherapeutic agent.
139. A method according to statement 138, wherein the subject has previously been treated with an agent selected from: a PHGDH inhibitor, a Slfn11 inhibitor, an Axl inhibitor, or an Akt3 inhibitor.
140. A method according to any one of statements 138-139, wherein the subject has previously been treated with an agent that is different to the agent capable of inhibiting or reversing EMT or chemotherapeutic agent for which the subject's susceptibility is being predicted.
141. A method according to any one of statements 101-136, wherein the subject has not previously been treated with an agent capable of inhibiting or reversing EMT or a chemotherapeutic agent.
142. A method according to statement 141, wherein the subject has not previously been treated with an Axl inhibitor.
143. A method according to statement 141, wherein the subject has not previously been treated with an Akt3 inhibitor.
144. A method according to any one of statements 101-143, wherein the sample is selected from: blood, serum, plasma, tissue biopsy, culture supernatant, or urine.
145. A method according to statement 144, wherein the sample is a blood, serum, or plasma sample.
146. A method according to statement 145, wherein the sample is a serum sample.
147. A method according to any one of statements 104-146, wherein the method further comprises a subsequent step of assessing the activity, expression, or amount of one or more biomarker in the subject, in which a sample profile is obtained after contacting the subject with an agent capable of inhibiting or reversing EMT or a chemotherapeutic agent.
148. A method according to statement 103, wherein the sample profile is obtained after contacting the subject with an agent capable of inhibiting or reversing EMT or a chemotherapeutic agent.
149. A method according to statement 147 or 148, wherein the control profile is obtained from the same subject prior to contacting said subject with the agent capable of inhibiting or reversing EMT or a chemotherapeutic agent.
150. A method according to statement 149, wherein an increase in the activity, expression, or amount of one or more biomarker in the sample profile as compared with the control profile is indicative of higher susceptibility to treatment with an agent capable of inhibiting or reversing EMT or a chemotherapeutic agent.
151. A method according to statement 149, wherein a decrease in the activity, expression, or amount of one or more biomarker in the sample profile as compared with the control profile is indicative of higher susceptibility to treatment with an agent capable of inhibiting or reversing EMT or a chemotherapeutic agent.
152. A method according to statement 149, wherein an increase in the activity, expression, or amount of one or more biomarker in the sample profile as compared with the control profile is indicative of lower susceptibility to treatment with an agent capable of inhibiting or reversing EMT or a chemotherapeutic agent.
153. A method according to statement 149, wherein a decrease in the activity, expression, or amount of one or more biomarker in the sample profile as compared with the control profile is indicative of lower susceptibility to treatment with an agent capable of inhibiting or reversing EMT or a chemotherapeutic agent.
154. A method according to statement 150, wherein the one or more biomarker includes at least Axl.
155. A method according to any one of statements 147-154, wherein assessing the activity, expression, or amount of one or more biomarker in the subject, or in a sample derived from the subject, comprises:
   contacting a sample, or an extract from the sample, with at least 1, 2, 3, 4, 5, 6, 7, 8, 9, 10 or more reagents for detecting the activity, expression, or amount of one or more biomarkers, each of which is selective for a particular biomarker; and
   detecting and/or quantifying said reagents for detecting.
156. A method according to statement 155, wherein assessing the expression or amount of one or more biomarker in the subject, or in a sample derived from the subject, comprises:
   contacting a sample, or an extract from the sample, with at least 1, 2, 3, 4, 5, 6, 7, 8, 9, 10 or more specific binding members, each of which selectively binds to a respective biomarker protein or mRNA; and
   detecting and/or quantifying formation of a complex formed by said specific binding member and said biomarker protein or mRNA.
157. A method according to statement 156, wherein the specific binding member comprises an antibody molecule or binding fragment thereof.
158. A method according to statement 156 or 157, wherein detecting formation of a complex formed by the specific binding member and the biomarker protein or mRNA is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.
159. A method according to any one of statements 156-158 wherein the expression or amount of the one or more biomarker is determined as the absolute amount of the respective biomarker protein or mRNA in the sample.
160. A method according to any one of statements 156-158 wherein the expression or amount of the one or more biomarker is determined as a relative amount of the respective biomarker protein or mRNA in the sample,
   optionally wherein the relative amount is determined relative to a reference protein or mRNA in the sample, the expression of which is not altered by AML.
161. A method according to any one of statements 148-160, wherein the agent capable of inhibiting or reversing EMT or chemotherapeutic agent is an Axl inhibitor, Akt3 inhibitor, Slfn11 inhibitor, or PHGDH inhibitor.
162. A method according to statement 161, wherein the agent capable of inhibiting or reversing EMT or chemotherapeutic agent is an Axl inhibitor.
163. A method according to statement 162, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
164. A method according to statement 163, wherein the Axl inhibitor is BGB324/R428/bemcentinib.
165. A method according to statement 163, wherein the Axl inhibitor is an anti-Axl antibody described in WO2015/193428, WO2015/193430, WO2016/097370, or WO2016/166296.
166. A method according to statement 161, wherein the agent capable of inhibiting or reversing EMT or chemotherapeutic agent is an Akt3 inhibitor.
167. A method according to statement 166, wherein the Akt3 inhibitor is an Akt3 inhibitor disclosed in WO2016/102672.
168. A method according to any one of statements 161-167, wherein the agent capable of inhibiting or reversing EMT is administered as a single agent.
169. A method according to any one of statements 161-167, wherein the agent capable of inhibiting or reversing EMT is administered in combination with a further cancer treatment.
170. A method according to statement 169, wherein the further cancer treatment is selected from the further cancer treatments listed in statement 130.
171. A method according to statement 169, wherein the further cancer treatment is a pyrimidine analogue, for example azacitidine, capecitabine, cytarabine, edatrexate, floxuridine, fluorouracil, gemcitabine, or troxacitabine, or a cytidine analogue, for example decitabine.
172. A method according to statement 171, wherein the further cancer treatment is cytarabine.
173. A method according to statement 171, wherein the further cancer treatment is decitabine.
174. A method according to any one of statements 148-173, wherein the subject is mammalian.
175. A method according to statement 174, wherein the subject is human.
176. A method according to any one of statements 148-175, wherein the subject was selected for treatment with the agent capable of inhibiting or reversing EMT or a chemotherapeutic agent, using a predictive method according to any one of statements 101-147.
177. A method according to any one of statements 148-176, wherein the sample is selected from: blood, serum, plasma, tissue biopsy, culture supernatant, or urine.
178. A method according to statement 177, wherein the sample is a blood, serum, or plasma sample.
179. A method according to statement 178, wherein the sample is a serum sample.
180. A method according to any one of statements 1-179, wherein the method is performed in vitro or ex vivo.
201. A method of selecting subjects having, suspected of having, or diagnosed with acute myelocytic leukemia (AML) for treatment with an agent capable of inhibiting or reversing EMT or a chemotherapeutic agent, the method comprising:
   identifying subjects susceptible to treatment with an agent capable of inhibiting or reversing EMT or a chemotherapeutic agent using a method according to any one of statements 101-180; and
   selecting thus identified subjects for treatment.
202. A method of selecting subjects having, suspected of having, or diagnosed with acute myelocytic leukemia (AML) for continued treatment with an agent capable of inhibiting or reversing EMT or a chemotherapeutic agent, the method comprising:
   identifying subjects susceptible to treatment with an agent capable of inhibiting or reversing EMT or a chemotherapeutic agent using a method according to any one of statements 148-180; and
   selecting thus identified subjects for treatment.
203. A method according to any one of statements 201-202, wherein treatment comprises administering to the subject a therapeutically effective amount of an agent capable of inhibiting or reversing EMT or a chemotherapeutic agent.
204. A method according to statement 203, wherein the treatment comprises an Axl inhibitor, Akt3 inhibitor, Slfn11 inhibitor, or PHGDH inhibitor.
205. A method according to statement 204, wherein the treatment comprises an Axl inhibitor.
206. A method according to statement 205, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
207. A method according to statement 206, wherein the Axl inhibitor is BGB324/R428/bemcentinib.
208. A method according to statement 206, wherein the Axl inhibitor is an anti-Axl antibody described in WO2015/193428, WO2015/193430, WO2016/097370, or WO2016/166296.
209. A method according to statement 204, wherein the treatment comprises an Akt3 inhibitor.
210. A method according to statement 209, wherein the Akt3 inhibitor is an Akt3 inhibitor disclosed in WO2016/102672.
211. A method according to any one of statements 203-210, wherein the treatment is administered as a single agent.
212. A method according to any one of statements 203-211, wherein the treatment is administered in combination with a further cancer treatment.
213. A method according to statement 212, wherein the further cancer treatment is selected from the further cancer treatments listed in statement 130.
214. A method according to statement 212, wherein the further cancer treatment is a pyrimidine analogue, for example azacitidine, capecitabine, cytarabine, edatrexate, floxuridine, fluorouracil, gemcitabine, or troxacitabine, or a cytidine analogue, for example decitabine.
215. A method according to statement 214, wherein the further cancer treatment is cytarabine.
216. A method according to statement 214, wherein the further cancer treatment is decitabine.
301. A diagnostic kit comprising 1, 2, 3, 4, 5, 6, 7, 8, 9, 10 or more reagents for detecting the activity, expression, or amount of one or more biomarkers, each of which is selective for a particular biomarker selected from the group consisting of: Axl, FGF-21, RAGE, CEA, FSH, MMP-10, Omentin, CA-19-9, LH, Haptoglobin, NAP-2, IgE, and/or CD40-L.
302. A test device comprising 1, 2, 3, 4, 5, 6, 7, 8, 9, 10 or more reagents for detecting the activity, expression, or amount of one or more biomarkers, each of which is selective for a particular biomarker selected from the group consisting of: Axl, FGF-21, RAGE, CEA, FSH, MMP-10, Omentin, CA-19-9, LH, Haptoglobin, NAP-2, IgE, and/or CD40-L.
303. A diagnostic kit or test device according to any one of statements 301 or 302, comprising:
   1, 2, 3, 4, 5, 6, 7, 8, 9, 10 or more specific binding members, each of which selectively binds to a biomarker selected from the group consisting of: Axl, FGF-21, RAGE, CEA, FSH, MMP-10, Omentin, CA-19-9, LH, Haptoglobin, NAP-2, IgE, and/or CD40-L; and
   one or more reagents for detecting said one or more specific binding members or one or more reagents for detecting and/or quantifying formation of a complex formed by said specific binding member and said biomarker.
304. A diagnostic kit or test device according to statement 303, wherein the specific binding member comprises an antibody molecule or binding fragment thereof.
305. A diagnostic kit or test device according to statement 303 or 304, wherein detecting formation of a complex formed by the specific binding member and the biomarker is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.
306. A diagnostic kit or test device according to any one of statements 301-305, wherein the kit or test device comprises no more than 15, 20, 25, 30, 40, or 50 reagents for detecting.
307. A diagnostic kit or test device according to any one of statements 301-306 for use in a method of predicting a cancer-related outcome in a subject.
308. A diagnostic kit for use, or test device for use, according to statement 307, wherein the cancer-related outcome is susceptibility to treatment with an agent capable of inhibiting or reversing EMT or a chemotherapeutic agent.
309. A diagnostic kit for use, or test device for use, according to statement 308, wherein the agent capable of inhibiting or reversing EMT or chemotherapeutic agent is an Axl inhibitor, Akt3 inhibitor, Slfn11 inhibitor, or PHGDH inhibitor.
310. A diagnostic kit for use, or test device for use, according to statement 308, wherein the agent capable of inhibiting or reversing EMT or chemotherapeutic agent is an Axl inhibitor.
311. A diagnostic kit for use, or test device for use, according to statement 310, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
312. A diagnostic kit for use, or test device for use, according to statement 311, wherein the Axl inhibitor is BGB324/R428/bemcentinib.
313. A diagnostic kit for use, or test device for use, according to statement 311, wherein the Axl inhibitor is an anti-Axl antibody described in WO2015/193428, WO2015/193430, WO2016/097370, or WO2016/166296.
314. A diagnostic kit for use, or test device for use, according to statement 308, wherein the agent capable of inhibiting or reversing EMT or chemotherapeutic agent is an Akt3 inhibitor.
315. A diagnostic kit for use, or test device for use, according to any one of statements 307-310, wherein the subject has, is suspected of having, or has been diagnosed with acute myelocytic leukemia (AML).
316. A diagnostic kit for use, or test device for use, according to any one of statements 307-315, wherein the method is a method according to any one of statements 101-180.
317. Use of a reagent for detecting the activity, expression, or amount of a biomarker selected from the group consisting of: Axl, FGF-21, RAGE, CEA, FSH, MMP-10, Omentin, CA-19-9, LH, Haptoglobin, NAP-2, IgE, and/or CD40-L, in a method of predicting a cancer-related outcome in a subject.
318. Use of 1, 2, 3, 4, 5, 6, 7, 8, 9, 10 or more reagents for detecting the activity, expression, or amount a biomarker, each of which is selective for a particular biomarker selected from the group consisting of: Axl, FGF-21, RAGE, CEA, FSH, MMP-10, Omentin, CA-19-9, LH, Haptoglobin, NAP-2, IgE, and/or CD40-L, in a method of manufacture of a diagnostic kit or test device for use in a method of predicting a cancer-related outcome in a subject.
319. A use according to statement 317 or 318, wherein the reagent for detecting comprises a specific binding member selective for a particular biomarker.
320. A use according to statement 317 or 318, wherein the method of predicting a cancer-related outcome in a subject is a method according to any one of statements 101-180.
401. Use of one or more of: Axl, FGF-21, RAGE, CEA, FSH, MMP-10, Omentin, CA-19-9, LH, Haptoglobin, NAP-2, IgE, and/or CD40-L, as a biomarker for determining if a subject is susceptible to treatment with an agent capable of inhibiting or reversing EMT or a chemotherapeutic agent.
402. A use according to statement 401, wherein:
   (i) a higher activity, expression, or amount of one or more of said biomarkers in the subject, or sample derived from the subject, compared with a control profile for the respective biomarker is indicative of susceptibility to treatment with an agent capable of inhibiting or reversing EMT or a chemotherapeutic agent; or
   (ii) a lower activity, expression, or amount of one or more of said biomarkers in the subject, or sample derived from the subject, compared with a control profile for the respective biomarker is indicative of susceptibility to treatment with an agent capable of inhibiting or reversing EMT or a chemotherapeutic agent.
403. A use according to statement 401, wherein:
   (i) an increase in the activity, expression, or amount of one or more biomarker relative to a control profile for the respective biomarker after contacting the subject with a cancer therapeutic agent is indicative of increased susceptibility to treatment with an agent capable of inhibiting or reversing EMT or a chemotherapeutic agent; or
   (ii) a decrease in the activity, expression, or amount of one or more biomarker relative to a control profile for the respective biomarker after contacting the subject with a cancer therapeutic agent is indicative of increased susceptibility to treatment with an agent capable of inhibiting or reversing EMT or a chemotherapeutic agent.
404. A use according to any one of statements 401-403, wherein the use is as a biomarker in a method according to any one of statements 101-180
501. A method of treating a subject having, suspected of having, or diagnosed with acute myelocytic leukemia (AML), the method comprising:
   identifying a subject susceptible to treatment using a method according to any one of statements 101-180; and
   administering to the subject a therapeutically effective amount of an agent capable of inhibiting or reversing EMT or a chemotherapeutic agent.
502. A method of treating a subject having, suspected of having, or diagnosed with acute myelocytic leukemia (AML), the method comprising:
   (a) obtaining a sample from the subject;
   (b) determining if the subject is susceptible to treatment with an agent capable of inhibiting or reversing EMT or a chemotherapeutic agent using a method according to any one of statements 101-180;
   (c) administering to the subject a therapeutically effective amount of an agent capable of inhibiting or reversing EMT or a chemotherapeutic agent.
503. A method of treating a subject having, suspected of having, or diagnosed with acute myelocytic leukemia (AML), the method comprising administering a therapeutically effective amount of an agent capable of inhibiting or reversing EMT or chemotherapeutic agent to a subject identified as susceptible to said treatment using a method according to any one of statements 101-180.
504. A method of treating a subject having, suspected of having, or diagnosed with acute myelocytic leukemia (AML), the method comprising:
   administering to the subject a therapeutically effective amount of an agent capable of inhibiting or reversing EMT or a chemotherapeutic agent; and
   determining if the subject is susceptible to said treatment using a method according to any one of statements 148-180.
505. A method according to statement 504, wherein the method comprises administering to the subject one or more further therapeutically effective amounts of an agent capable of inhibiting or reversing EMT or a chemotherapeutic agent provided that the subject is identified as susceptible to said treatment.
506. An agent capable of inhibiting or reversing EMT or chemotherapeutic agent for use in a method of treating a subject having, suspected of having, or diagnosed with acute myelocytic leukemia (AML),
   wherein the method is a method according to any one of statements 501-505.
507. Use of an agent capable of inhibiting or reversing EMT or chemotherapeutic agent for use in a method of manufacture of a medicament for use in a method of treating a subject having, suspected of having, or diagnosed with acute myelocytic leukemia (AML),
   wherein the method of treatment is a method according to any one of statements 501-505.
508. A method, agent for use, or use according to any one of statements 501-507, wherein the agent capable of inhibiting or reversing EMT or chemotherapeutic agent comprises an Axl inhibitor, Akt3 inhibitor, Slfn11 inhibitor, or PHGDH inhibitor.
509. A method, agent for use, or use according to statement 508, wherein the agent capable of inhibiting or reversing EMT or chemotherapeutic agent comprises an Axl inhibitor.
510. A method, agent for use, or use according to statement 509, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
511. A method, agent for use, or use according to statement 510, wherein the Axl inhibitor is BGB324/R428/bemcentinib.
512. A method, agent for use, or use according to statement 510, wherein the Axl inhibitor is an anti-Axl antibody described in WO2015/193428, WO2015/193430, WO2016/097370, or WO2016/166296.
512. A method, agent for use, or use according to statement 508, wherein the agent capable of inhibiting or reversing EMT or chemotherapeutic agent comprises an Akt3 inhibitor.
513. A method, agent for use, or use according to statement 512, wherein the Akt3 inhibitor is an Akt3 inhibitor disclosed in WO2016/102672.
514. A method, agent for use, or use according to any one of statements 501-513, wherein the agent capable of inhibiting or reversing EMT or chemotherapeutic agent is administered as a single agent.
515. A method, agent for use, or use according to any one of statements 501-513, wherein the agent capable of inhibiting or reversing EMT or chemotherapeutic agent is administered in combination with a further cancer treatment.
516. A method, agent for use, or use according to statement 515, wherein the further cancer treatment is selected from the further cancer treatments listed in statement 130.
517. A method, agent for use, or use according to statement 515, wherein the further cancer treatment is a pyrimidine analogue, for example azacitidine, capecitabine, cytarabine, edatrexate, floxuridine, fluorouracil, gemcitabine, or troxacitabine, or a cytidine analogue, for example decitabine.
518. A method, agent for use, or use according to statement 517, wherein the further cancer treatment is cytarabine.
519. A method, agent for use, or use according to statement 517, wherein the further cancer treatment is decitabine.

Facet 2: Myelodysplastic Syndrome (MDS)

Myelodysplastic syndromes (MDS) are a diverse group of clonal and malignant myeloid disorders characterized by ineffective hematopoiesis, resultant peripheral cytopenias, and increased risk of progression to acute myeloid leukemia. Chemotherapeutic drug therapies for MDS include lenalidomide, antithymocyte globulin, azacitidine, and decitabine. However, current therapies are limited in their efficacy, and their remains a need for combination and targeted therapies for treatment of MDS (Ma et al, 2018).

Accordingly, robust biomarkers for predicting cancer-related outcomes in a subject having MDS, for example susceptibility to treatment with an agent capable of inhibiting or reversing EMT or a chemotherapeutic agent, would be useful in identifying subjects who are most likely to respond to or benefit from treatment with a particular agent/regimen.

A “responder” subject as described herein is a subject who is likely to, or who does, respond to treatment (or benefit from treatment) with an agent capable of inhibiting or reversing EMT or a chemotherapeutic agent, for example the small molecule Axl kinase inhibitor BGB324/R428/bemcentinib when administered either as a single agent or in combination with another cancer treatment.

Correspondingly, a “non-responder” subject as described herein is a subject who is not likely to, or who does not, respond to treatment (or benefit from treatment) with an agent capable of inhibiting or reversing EMT or a chemotherapeutic agent, for example the small molecule Axl kinase inhibitor BGB324/R428/bemcentinib when administered either as a single agent or in combination with another cancer treatment.

To “respond to treatment” or “benefit from treatment” as used herein means to experience an overall clinical benefit from the treatment. This overall clinical benefit can be any of: prolonged survival, partial or complete disease remission, (for example, as assessed by % bone marrow myeloblasts and/or normal maturation of cell lines), slowing or absence of disease progression (for example, as assessed by change in % bone marrow myeloblasts), improved quality of life (for example, as assessed using a health-related quality of life questionnaire such as a Functional Assessment of Cancer Therapy (FACT) questionnaire), progression-free survival, hematologic improvement (for example: increased blood haemoglobin, platelet count, and/or neutrophil count), bone marrow response (for example: bone marrow with 5% myeloblasts; 30%, 40%, 50% or more reduction in bone marrow myeloblasts; absence of circulating myeloblasts and myeloblasts with Auer rods; absence of extramedullary disease), hematologic recovery (for example: ≥11 g/dL haemoglobin, ≥100×10⁹/L platelets, and/or ≥1×10⁹/L neutrophils in peripheral blood), negative response for a genetic marker (for example, CEBPA, NPM1, or FLT3), or any other positive patient outcome.

Biomarkers

The term “marker” or “biomarker” is used herein to refer to a gene or protein whose expression in a subject or sample derived from a subject is altered or modulated, for example, up or down regulated, in cancer. Where the biomarker is a protein, modulation or alteration of expression encompasses modulation through different post-translational modifications.

In accordance with any aspect of this facet of the present disclosure the biomarkers described herein may be defined as follows, wherein:
an Axl biomarker comprises an amino acid sequence having at least 70%, 80%, 90%, 95%, 99% or 100% sequence identity with the full-length of the amino acid sequence disclosed at UniProt accession no: P30530-1 or P30530-2 (entry version 196) or a fragment thereof;
a Transferrin receptor protein 1 (TFR1) biomarker comprises an amino acid sequence having at least 70%, 80%, 90%, 95%, 99% or 100% sequence identity with the full-length of the amino acid sequence disclosed at UniProt accession no: P02786 (entry version 222) or a fragment thereof;
a FASLG Receptor (FAS) biomarker comprises an amino acid sequence having at least 70%, 80%, 90%, 95%, 99% or 100% sequence identity with the full-length of the amino acid sequence disclosed at UniProt accession no: P25445-1, P25445-2, P25445-3, P25445-4, P25445-5, P25445-6, or P25445-7 (entry version 227) or a fragment thereof;
an Interleukin-8 (IL-8) biomarker comprises an amino acid sequence having at least 70%, 80%, 90%, 95%, 99% or 100% sequence identity with the full-length of the amino acid sequence disclosed at UniProt accession no: P10145 (entry version 210) or a fragment thereof;
a Tamm-Horsfall Urinary Glycoprotein (THP) biomarker comprises an amino acid sequence having at least 70%, 80%, 90%, 95%, 99% or 100% sequence identity with the full-length of the amino acid sequence disclosed at UniProt accession no: P07911-1, P07911-2, P07911-3, P07911-4, or P07911-5 (entry version 182) or a fragment thereof;
a TNF-Related Apoptosis-Inducing Ligand Receptor 3 (TRAIL-R3) biomarker comprises an amino acid sequence having at least 70%, 80%, 90%, 95%, 99% or 100% sequence identity with the full-length of the amino acid sequence disclosed at UniProt accession no: 014798 (entry version 152) or a fragment thereof;
a Macrophage-Derived Chemokine (MDC) biomarker comprises an amino acid sequence having at least 70%, 80%, 90%, 95%, 99% or 100% sequence identity with the full-length of the amino acid sequence disclosed at UniProt accession no: 000626 (entry version 153) or a fragment thereof;
an Antileukoproteinase (ALP) biomarker comprises an amino acid sequence having at least 70%, 80%, 90%, 95%, 99% or 100% sequence identity with the full-length of the amino acid sequence disclosed at UniProt accession no: P03973 (entry version 184) or a fragment thereof;
a Trefoil Factor 3 (TFF3) biomarker comprises an amino acid sequence having at least 70%, 80%, 90%, 95%, 99% or 100% sequence identity with the full-length of the amino acid sequence disclosed at UniProt accession no: Q07654 (entry version 161) or a fragment thereof;
a Fatty Acid-Binding Protein, adipocyte (FABP) biomarker comprises an amino acid sequence having at least 70%, 80%, 90%, 95%, 99% or 100% sequence identity with the full-length of the amino acid sequence disclosed at UniProt accession no: P15090 (entry version 183) or a fragment thereof;
a von Willebrand Factor (vWF) biomarker comprises an amino acid sequence having at least 70%, 80%, 90%, 95%, 99% or 100% sequence identity with the full-length of the amino acid sequence disclosed at UniProt accession no: P04275-1 or P04275-2 (entry version 233) or a fragment thereof;
a Vitamin D-Binding Protein (VDBP) biomarker comprises an amino acid sequence having at least 70%, 80%, 90%, 95%, 99% or 100% sequence identity with the full-length of the amino acid sequence disclosed at UniProt accession no: P02774-1, P02774-2, or P02774-3 (entry version 201) or a fragment thereof;
a Cystatin-B biomarker comprises an amino acid sequence having at least 70%, 80%, 90%, 95%, 99% or 100% sequence identity with the full-length of the amino acid sequence disclosed at UniProt accession no: P04080 (entry version 201) or a fragment thereof;
a Uteroglobin biomarker comprises an amino acid sequence having at least 70%, 80%, 90%, 95%, 99% or 100% sequence identity with the full-length of the amino acid sequence disclosed at UniProt accession no: P11684 (entry version 163) or a fragment thereof;
a Fibrinogen biomarker comprises an amino acid sequence having at least 70%, 80%, 90%, 95%, 99% or 100% sequence identity with the full-length of the amino acid sequence disclosed at UniProt accession no: P02671-1 or P02671-2 (entry version 227) or a fragment thereof;
an Epidermal Growth Factor Receptor (EGFR) biomarker comprises an amino acid sequence having at least 70%, 80%, 90%, 95%, 99% or 100% sequence identity with the full-length of the amino acid sequence disclosed at UniProt accession no: Q504U8 (entry version 138) or a fragment thereof; and/or
a Leptin biomarker comprises an amino acid sequence having at least 70%, 80%, 90%, 95%, 99% or 100% sequence identity with the full-length of the amino acid sequence disclosed at UniProt accession no: P41159 (entry version 176) or a fragment thereof.

The biomarkers described herein may also be defined as a nucleic acid (either DNA or RNA) having at least 70%, 80%, 90%, 95%, 99% or 100% sequence identity with the full-length nucleic acid sequence encoding any of the above amino acid sequences.

Identity may be as defined using sequence comparisons made using FASTA and FASTP (see Pearson & Lipman, 1988. Methods in Enzymology 183: 63-98). Parameters are preferably set, using the default matrix, as follows: Gapopen (penalty for the first residue in a gap): −12 for proteins/-16 for DNA; Gapext (penalty for additional residues in a gap): −2 for proteins/-4 for DNA; KTUP word length: 2 for proteins/6 for DNA.

Pre-Treatment Predictive Biomarkers and Methods

By analysing patient data obtained from clinical trial BGBC003 (NCT02488408), the authors have discovered that, prior to treatment of subjects having MDS, certain of the biomarkers described herein are present in the serum of “responder” subjects at higher levels than in the serum of “non-responder” subjects—see Example 1. Similarly, prior to treatment of the subject certain of the biomarkers described herein are present in the serum of “responder” subjects at lower levels than in the serum of “non-responder” subjects. Thus, by assessing the activity, expression, or amount of one or more of the biomarkers described herein prior to treatment with an agent capable of inhibiting or reversing EMT or a chemotherapeutic agent it is possible to determine the likelihood of that subject being susceptible or responsive to treatment with said agent.

Accordingly, in a first aspect of this facet, the disclosure provides a method of predicting a cancer-related outcome in a subject having, suspected of having, or diagnosed with myelodysplastic syndrome (MDS), the method comprising assessing the activity, expression, or amount of one or more of the biomarkers described herein in the subject, or in a sample derived from the subject.

In some embodiments the one or more biomarker is selected from the group consisting of: Axl, Transferrin receptor protein 1 (TFR1), FASLG Receptor (FAS), Interleukin-8 (IL-8), Tamm-Horsfall Urinary Glycoprotein (THP), TNF-Related Apoptosis-Inducing Ligand Receptor 3 (TRAIL-R3), Macrophage-Derived Chemokine (MDC), Antileukoproteinase (ALP), Trefoil Factor 3 (TFF3), Fatty Acid-Binding Protein, adipocyte (FABP), von Willebrand Factor (vWF), Vitamin D-Binding Protein (VDBP), Cystatin-B, Uteroglobin, Fibrinogen, Epidermal Growth Factor Receptor (EGFR), and/or Leptin.

A “cancer-related outcome” as described herein is a clinical prediction or prognosis associated with cancer. In some embodiments the cancer-related outcome is susceptibility to treatment with an agent capable of inhibiting or reversing EMT or a chemotherapeutic agent.

In some embodiments the method comprises assessing the activity, expression, or amount of one or more of the biomarkers described herein in the subject, or in a sample derived from the subject, to obtain a sample profile of the one or more biomarker; and, making a prediction based on the sample profile of the one or more biomarker.

A “sample profile” as used herein is a profile of numerical values or numerical ranges corresponding to the activity, expression, or amount of respective biomarkers determined in the subject or sample derived from the subject.

In some embodiments of the method the prediction is made by comparing the sample profile to a control profile. A “control profile” as used herein is a profile of numerical values or numerical ranges corresponding to the activity, expression, or amount of the same biomarkers as for the sample profile, determined in a control subject or population of control subjects. Suitable control profiles are described in more detail below.

Prior to treatment of a subject, certain of the biomarkers described herein have been found to exhibit up-regulated expression in “responder” subjects—that is, certain of the biomarkers described herein have been found to be present in the serum of “responder” subjects at higher levels than in the serum of “non-responder” subjects. Similarly, prior to treatment of a subject, certain of the biomarkers described herein have been found to exhibit down-regulated expression in “responder” subjects—that is, certain of the biomarkers described herein have been found to be present in the serum of “responder” subjects at lower levels than in the serum of “non-responder” subjects.

This difference in expression level between “responder” and “non-responder” subjects can also be expressed in the opposite way. That is to say, prior to treatment of a subject, certain of the biomarkers described herein have been found to exhibit down-regulated expression in “non-responder” subjects—that is, certain of the biomarkers described herein have been found to be present in the serum of “non-responder” subjects at lower levels than in the serum of “responder” subjects. Similarly, prior to treatment of a subject, certain of the biomarkers described herein have been found to exhibit up-regulated expression in “non-responder” subjects—that is, certain of the biomarkers described herein have been found to be present in the serum of “non-responder” subjects at higher levels than in the serum of “responder” subjects.

The discovery of these differential patterns of expression mean that, by assessing the activity, expression, or amount of one or more of the biomarkers described herein prior to treatment of a subject with an agent, it is possible to determine the likelihood of that subject being susceptible or responsive to treatment with said agent. Accordingly, in particularly preferred embodiments the sample profile is obtained before the subject is contacted with or administered the agent capable of inhibiting or reversing EMT or a chemotherapeutic agent.

A subject who is “susceptible to treatment” with an agent capable of inhibiting or reversing EMT or a chemotherapeutic agent is one who is likely to, or who does, respond to treatment (or benefit from treatment) with an agent capable of inhibiting or reversing EMT or a chemotherapeutic agent. That is, a subject who is “susceptible to treatment” with an agent is a “responder” subject for that agent, as defined above. A prediction that is “indicative of susceptibility” is one which suggests or points towards the likelihood that a subject will respond to or benefit from treatment.

In some embodiments the predictive methods of the disclosure comprise assessing the activity, expression, or amount of one or more of the biomarkers described herein in the subject, or in a sample derived from the subject, to obtain a sample profile of the one or more biomarker; and, making a prediction based on the sample profile of the one or more biomarker. In some embodiments of the method the prediction is made by comparing the sample profile to a control profile, wherein the control profile is a profile of numerical values or numerical ranges corresponding to the activity, expression, or amount of the same biomarkers as for the sample profile, determined in a control subject or population of control subjects.

In some embodiments the control profile may be obtained from a population of control subjects having MDS. In some embodiments the control profile may be obtained from a population of control subjects not having MDS. In other embodiments the control profile may be obtained from a control subject or population of control subjects having MDS and previously found to lack susceptibility to treatment with an agent capable of inhibiting or reversing EMT or a chemotherapeutic agent (that is, non-responder subjects).

In some embodiments the control profile may a predetermined profile of biomarker expression, activity, or amount values, for example a profile of “mean” values, “threshold” values, or “standard ranges” of values. This predetermined profile of biomarker expression, activity, or amount values can be obtained, for example, from a control subject or population of control subjects having MDS, a control subject or population of control subjects not having MDS, or a control subject or population of control subjects having MDS and previously found to lack susceptibility to treatment with an agent capable of inhibiting or reversing EMT or a chemotherapeutic agent (that is, non-responder subjects).

In some other embodiments the control profile may be obtained from a control subject or population of control subjects having MDS and previously found to be susceptible to treatment with an agent capable of inhibiting or reversing EMT or a chemotherapeutic agent (that is, responder subjects). In some embodiments the control profile may a predetermined profile of biomarker expression, activity, or amount values, for example a profile of “threshold”, values or “standard ranges” of values. This predetermined profile of biomarker expression, activity, or amount values can be obtained, for example, from a control subject or population of control subjects having MDS and previously found to be susceptible to treatment with an agent capable of inhibiting or reversing EMT or a chemotherapeutic agent (that is, responder subjects).

In some other embodiments the control profile may be obtained from a control sample having a known amount (for example a “threshold” amount) of the same biomarkers as will be determined in the sample profile. This threshold amount can be obtained, for example, from non-responder or responder subjects as outlined above.

The skilled person is readily able to determine suitable “mean”, “threshold”, or “standard range” values for a particular biomarker.

For example, a “mean” value may be determined by: assessing the activity, expression, or amount of a particular biomarker in a population of subjects having (or not having) MDS; and, averaging the biomarker activity, expression, or amount determined to determine a “mean” value.

For example, a “mean” value may be determined by: assessing the activity, expression, or amount of a particular biomarker in a population of subjects; determining which subjects in the population are susceptible to treatment with an agent capable of inhibiting or reversing EMT or a chemotherapeutic agent; and, averaging the biomarker activity, expression, or amount determined in subjects found to be susceptible to treatment with an agent capable of inhibiting or reversing EMT or a chemotherapeutic agent (that is, responder subjects) to determine a “mean” value.

For example, a “threshold” value may be determined by: assessing the activity, expression, or amount of a particular biomarker in a population of subjects; determining which subjects in the population are susceptible to treatment with an agent capable of inhibiting or reversing EMT or a chemotherapeutic agent; and, selecting as appropriate the highest or lowest biomarker activity, expression, or amount determined in a subject found to be susceptible to treatment with an agent capable of inhibiting or reversing EMT or a chemotherapeutic agent (that is, a responder subject) as the “threshold value”.

For example, a “standard range” of values may be determined by: assessing the activity, expression, or amount of a particular biomarker in a population of subjects having (or not having) MDS; and, determining a “standard range” of values based on this assessment.

For example, a “standard range” of values may be determined by: assessing the activity, expression, or amount of a particular biomarker in a population of subjects; determining which subjects in the population are susceptible to treatment with an agent capable of inhibiting or reversing EMT or a chemotherapeutic agent; and, determining a “standard range” of values for subjects found to be susceptible to treatment with an agent capable of inhibiting or reversing EMT or a chemotherapeutic agent (that is, a responder subject) based on this assessment.

Where the control profile is a predetermined profile of biomarker expression, activity, or amount values, this may be provided as a “look-up table” or data record. Predetermined control profiles of this type may be a profile of numerical values or numerical ranges corresponding to a measure of central tendency (such as an average, median or mean) amount of the respective biomarkers determined in a plurality of control subjects (for example non-responder or responder subjects as outlined above). The skilled person is readily able to determine both a measure of central tendency and a measure of variability within a population using standard protein quantification techniques and suitable statistical tools.

Accordingly, in embodiments in which the control profile is: obtained from a population of control subjects having MDS; obtained from a population of control subjects not having MDS; obtained from a control subject or population of control subjects having MDS and previously found to lack susceptibility to treatment with an agent capable of inhibiting or reversing EMT or a chemotherapeutic agent (that is non-responder subjects); a predetermined profile of biomarker expression, activity, or amount values (for example a profile of “average, median, or mean” values or “standard ranges” of values) obtained from non-responder subjects; obtained from a control sample having a known “average, median, or mean” value of biomarkers indicative of non-responder subjects; a predetermined profile of biomarker expression, activity, or amount “threshold” values obtained from responder subjects; or, obtained from a control sample having a known “threshold” value of biomarkers indicative of responder subjects; then, a higher activity, expression, or amount of one or more of the biomarkers described herein in a sample profile as compared with the control profile may be indicative of susceptibility to treatment with an agent capable of inhibiting or reversing EMT or a chemotherapeutic agent. Similarly, a lower activity, expression, or amount of one or more of the biomarkers described herein in the sample profile as compared with a control profile may be indicative of susceptibility to treatment with an agent capable of inhibiting or reversing EMT or a chemotherapeutic agent.

In some such embodiments, the method may comprise assessing the activity, expression, or amount of one or more of biomarker selected from: THP, TRAIL-R3, MDC, ALP, TFF3, FABP, vWF, VDBP, Cystatin-B, Uteroglobin, Fibrinogen, EGFR, and/or Leptin; wherein a higher activity, expression, or amount of one or more of said biomarkers in the sample profile as compared with the control profile is indicative of susceptibility to treatment with an agent capable of inhibiting or reversing EMT or a chemotherapeutic agent.

In some such embodiments, the method may comprise assessing the activity, expression, or amount of one or more of biomarker selected from: Axl, TFR1, FAS, and/or IL-8; wherein a lower activity, expression, or amount of one or more of said biomarkers in the sample profile as compared with the control profile is indicative of susceptibility to treatment with an agent capable of inhibiting or reversing EMT or a chemotherapeutic agent.

In some such embodiments: a lower activity, expression, or amount of Axl; and/or a lower activity, expression, or amount of TFR1; and/or a lower activity, expression, or amount of FAS; and/or a lower activity, expression, or amount of IL-8; and/or a higher activity, expression, or amount of THP; and/or a higher activity, expression, or amount of TRAIL-R3; and/or a higher activity, expression, or amount of MDC; and/or a higher activity, expression, or amount of ALP; and/or a higher activity, expression, or amount of TFF3; and/or a higher activity, expression, or amount of FABP; and/or a higher activity, expression, or amount of vWF; and/or a higher activity, expression, or amount of VDBP; and/or a higher activity, expression, or amount of Cystatin-B; and/or a higher activity, expression, or amount of Uteroglobin; and/or a higher activity, expression, or amount of Fibrinogen; and/or a higher activity, expression, or amount of EGFR; and/or a higher activity, expression, or amount of Leptin; in the sample profile as compared with the control profile is indicative of susceptibility to treatment with an agent capable of inhibiting or reversing EMT or a chemotherapeutic agent.

In embodiments in which the control profile is: obtained from a control subject or population of control subjects having MDS and previously found to be susceptible to treatment with an agent capable of inhibiting or reversing EMT or a chemotherapeutic agent (that is, responder subjects); then, if the expression, activity, or amount values in the sample profile are above a “threshold” value typical of a responder subject (that is, a subject having MDS and previously found to be susceptible to treatment with an agent capable of inhibiting or reversing EMT or a chemotherapeutic agent) this is indicative of susceptibility to treatment with an agent capable of inhibiting or reversing EMT or a chemotherapeutic agent. Similarly, if the expression, activity, or amount values in the sample profile are below the “threshold” value typical of a responder subject (that is, a subject having MDS and previously found to be susceptible to treatment with an agent capable of inhibiting or reversing EMT or a chemotherapeutic agent) this is indicative of a lack of susceptibility to treatment with an agent capable of inhibiting or reversing EMT or a chemotherapeutic agent.

In embodiments in which the control profile is: a predetermined profile of “standard ranges” of biomarker expression, activity, or amount values obtained from responder subjects; then, if the expression, activity, or amount values in the sample profile are within the “standard range” of values typical of a responder subject (that is, a subject having MDS and previously found to be susceptible to treatment with an agent capable of inhibiting or reversing EMT or a chemotherapeutic agent) this is indicative of susceptibility to treatment with an agent capable of inhibiting or reversing EMT or a chemotherapeutic agent. Similarly, if the expression, activity, or amount values in the sample profile are outside the “standard range” of values typical of a responder subject (that is, a subject having MDS and previously found to be susceptible to treatment with an agent capable of inhibiting or reversing EMT or a chemotherapeutic agent) this is indicative of a lack of susceptibility to treatment with an agent capable of inhibiting or reversing EMT or a chemotherapeutic agent.

In embodiments in which the control profile is: a predetermined profile of “standard ranges” of biomarker expression, activity, or amount values obtained from non-responder subjects; then, if the expression, activity, or amount values in the sample profile are within the “standard range” of values typical of a non-responder subject (that is, a subject having MDS and previously found to lack susceptibility to treatment with an agent capable of inhibiting or reversing EMT or a chemotherapeutic agent) this is indicative of a lack of susceptibility to treatment with an agent capable of inhibiting or reversing EMT or a chemotherapeutic agent. Similarly, if the expression, activity, or amount values in the sample profile are outside the “standard range” of values typical of a non-responder subject (that is, a subject having MDS and previously found to lack susceptibility to treatment with an agent capable of inhibiting or reversing EMT or a chemotherapeutic agent) this may be indicative of susceptibility to treatment with an agent capable of inhibiting or reversing EMT or a chemotherapeutic agent.

In embodiments in which the control profile is: obtained from a population of control subjects not having MDS; then, if the expression, activity, or amount values in the sample profile are outside a “standard range” of values typical of subjects not having MDS, this is indicative of susceptibility to treatment with an agent capable of inhibiting or reversing EMT or a chemotherapeutic agent. Similarly, if the expression, activity, or amount values in the sample profile are within a “standard range” of values typical of subjects not having MDS, this is indicative of a lack of susceptibility to treatment with an agent capable of inhibiting or reversing EMT or a chemotherapeutic agent. In such cases, an expression, activity or amount value may be outside the “standard range” of values when it is either above the upper limit of normal or below the lower limit of normal for a given biomarker. The upper limit of normal and lower limit of normal are the limits of the normal distribution of a given biomarker in a population of control subjects not having MDS.

The skilled person is readily able to determine whether the sample profile is indicative of susceptibility or lack of susceptibility to treatment with an agent capable of inhibiting or reversing EMT or a chemotherapeutic agent using suitable statistical tools and comparison with the appropriate control.

In some embodiments the sample profile may be compared with two or more control profiles as described above.

As used herein, the term “one or more” means 1, 2, 3, 4, 5, 6, 7, 8, 9, 10, 11, 12, 13, 14, 15, 16, 17, 18, 19, or 20 of the biomarkers described herein. In some embodiments the one or more biomarker is Axl. In some embodiments the one or more biomarker is TFR1. In some embodiments the one or more biomarker is FAS. In some embodiments the one or more biomarker is IL-8. In some embodiments the one or more biomarker is THP. In some embodiments the one or more biomarker is TRAIL-R3. In some embodiments the one or more biomarker is MDC. In some embodiments the one or more biomarker is ALP. In some embodiments the one or more biomarker is TFF3. In some embodiments the one or more biomarker is FABP. In some embodiments the one or more biomarker is vWF. In some embodiments the one or more biomarker is VDBP. In some embodiments the one or more biomarker is Cystatin B. In some embodiments the one or more biomarker is Uteroglobin. In some embodiments the one or more biomarker is Fibrinogen. In some embodiments the one or more biomarker is EGFR. In some embodiments the one or more biomarker is Leptin.

In some embodiments the method may comprise assessing the activity, expression, or amount of at least 2, 3, 4, 5, 6, 7, 8, 9, 10, 11, 12, 13, 14, 15, 20 of said biomarkers in the subject, or in a sample derived from the subject. In some embodiments the method may comprise assessing the activity, expression, or amount of Axl and at least 1, 2, 3, 4, 5, 6, 7, 8, 9, 10, 11, 12, 13, 14, 15, 20 further biomarkers described herein in the subject, or in a sample derived from the subject.

Subject

A “subject” as referred to herein is any species of animal classified as a mammal, and includes but is not restricted to, domestic and farm animals, primates and humans. In some preferred embodiments the subject is a human of any sex or race. In some embodiments, the human is an adult human.

In some embodiments the predictive methods described herein are performed before the subject receives a treatment or course of treatment for MDS. In other embodiments the predictive method is performed during or after a treatment or course of treatment for MDS. In some embodiments the treatment or course of treatment for MDS is an agent capable of inhibiting or reversing EMT. In other embodiments the treatment or course of treatment for MDS is a chemotherapeutic treatment that is not an agent capable of inhibiting or reversing EMT.

In some embodiments of the predictive methods described herein the subject has previously been treated with an agent capable of inhibiting or reversing EMT or a chemotherapeutic agent. In some embodiments the subject has previously been treated with an agent selected from: a PHGDH inhibitor, a Slfn11 inhibitor, an Axl inhibitor, or an Akt3 inhibitor.

In some embodiments of the predictive methods described herein the subject has previously been treated with an agent that is different to the agent capable of inhibiting or reversing EMT or chemotherapeutic agent for which the subject's response is being predicted. That is, the subject has previously been treated with an agent that is not the agent which the predictive method is determining the subject's susceptibility to. Accordingly, in some embodiments, the subject has not previously been treated with an agent capable of inhibiting or reversing EMT or a chemotherapeutic agent. In some preferred embodiments, the subject has not previously been treated with an Axl inhibitor. In other preferred embodiments, the subject has not previously been treated with an Akt3 inhibitor.

Post-Treatment Changes in Biomarkers

In some embodiments of this aspect of the disclosure, the predictive method further comprises a subsequent step of assessing the activity, expression, or amount of one or more biomarker in the subject, or in a sample derived from the subject, to obtain a second sample profile of one or more of the biomarkers described herein after the subject has received a treatment or course of treatment an agent capable of inhibiting or reversing EMT or a chemotherapeutic agent.

That is, in some embodiments, the predictive method comprises:
(a) determining if a subject is susceptible to treatment with an agent capable of inhibiting or reversing EMT or a chemotherapeutic agent by assessing the activity, expression, or amount of one or more biomarker in the subject, as outlined above;
(b) administering to the subject a therapeutically effective amount of an agent capable of inhibiting or reversing EMT or a chemotherapeutic agent; and
(c) subsequently assessing the activity, expression, or amount of one or more biomarker in the subject, or in a sample derived from the subject.

In some embodiments, the predictive method comprises:
(a) determining if a subject is susceptible to treatment with an agent capable of inhibiting or reversing EMT or a chemotherapeutic agent by assessing the activity, expression, or amount of one or more biomarker in the subject, or in a sample derived from the subject, wherein the one or more biomarker is selected from the group consisting of: Axl, TFR1, FAS, IL-8, THP, TRAIL-R3, MDC, ALP, TFF3, FABP, vWF, VDBP, Cystatin-B, Uteroglobin, Fibrinogen, EGFR, and/or Leptin;
(b) administering to the subject a therapeutically effective amount of an agent capable of inhibiting or reversing EMT or a chemotherapeutic agent; and
(c) subsequently assessing the activity, expression, or amount of one or more biomarker in the subject, or in a sample derived from the subject.

In some preferred embodiments the methods comprise subsequently assessing the activity, expression, or amount of Axl in the subject, or in a sample derived from the subject. In some embodiments an increase in the activity, expression, or amount of Axl in the second sample profile as compared with a control profile after contacting the subject with a cancer therapeutic agent is indicative of higher susceptibility to treatment with an agent capable of inhibiting or reversing EMT or a chemotherapeutic agent. In some preferred embodiments the control profile is obtained from the same subject prior to treatment with an agent capable of inhibiting or reversing EMT or a chemotherapeutic agent.

Post-Treatment Predictive Biomarkers and Methods

By analysing patient data obtained from clinical trial BGBC003 (NCT02488408), the authors have discovered that, following treatment of subjects having MDS with an agent capable of inhibiting or reversing EMT or a chemotherapeutic agent, the levels of certain of the biomarkers described herein increases in the serum of “responder” subjects but not in the serum of “non-responder” subjects—see Example 1. Similarly, following treatment with an agent capable of inhibiting or reversing EMT or a chemotherapeutic agent, the levels of certain of the biomarkers described herein decreases in the serum of “responder” subjects but not in the serum of “non-responder” subjects. Thus, by assessing the activity, expression, or amount of one or more of the biomarkers described herein prior to and following treatment with an agent capable of inhibiting or reversing EMT or a chemotherapeutic agent it is possible to determine or confirm that the subject is susceptible or responsive to treatment with said agent.

Accordingly, in a second aspect of this facet, the disclosure provides a method of predicting a cancer-related outcome in a subject having, suspected of having, or diagnosed with myelodysplastic syndrome (MDS), the method comprising assessing the activity, expression, or amount of one or more of the biomarkers described herein in a subject, or in a sample derived from the subject after the subject has been contacted with an agent capable of inhibiting or reversing EMT or a chemotherapeutic agent. In some embodiments the cancer-related outcome is susceptibility to treatment with an agent capable of inhibiting or reversing EMT or a chemotherapeutic agent.

In some embodiments the method comprises assessing the activity, expression, or amount of one or more of the biomarkers described herein in the subject, or in a sample derived from the subject, to obtain a sample profile of the one or more biomarker; and, making a prediction based on the sample profile of the one or more biomarker. In some embodiments of the method the prediction is made by comparing the sample profile to a control profile. In preferred embodiments the sample profile is obtained after contacting the subject with an agent capable of inhibiting or reversing EMT or a chemotherapeutic agent.

A “control profile” as used herein is a profile of numerical values or numerical ranges corresponding to the activity, expression, or amount of the same biomarkers as for the sample profile, determined in a control subject or population of control subjects.

In preferred embodiments of this aspect of the disclosure the control profile is obtained from the same subject prior to treatment with an agent capable of inhibiting or reversing EMT or a chemotherapeutic agent. In some embodiments the control profile is obtained from a population of control subjects having MDS. In other embodiments, the control profile is a predetermined profile of biomarker expression, activity, or amount values, for example a profile of “threshold” values or “standard ranges” of values.

Accordingly, in embodiments in which the control profile is obtained from the same subject prior to treatment with an agent capable of inhibiting or reversing EMT or a chemotherapeutic agent, an increase in the activity, expression, or amount of one or more biomarker in the sample profile as compared with a control profile after contacting the subject with an agent capable of inhibiting or reversing EMT or a cancer therapeutic agent may be indicative of susceptibility to treatment with an agent capable of inhibiting or reversing EMT or a chemotherapeutic agent. In such embodiments, a decrease in the activity, expression, or amount of one or more biomarker in the sample profile as compared with a control profile after contacting the subject with a cancer therapeutic agent may be indicative of higher susceptibility to treatment with an agent capable of inhibiting or reversing EMT or a chemotherapeutic agent.

In some such embodiments, an increase in the activity, expression, or amount of one or more biomarker in the sample profile as compared with a control profile after contacting the subject with a cancer therapeutic agent may be indicative of lower susceptibility to treatment with an agent capable of inhibiting or reversing EMT or a chemotherapeutic agent. In some such embodiments, a decrease in the activity, expression, or amount of one or more biomarker in the sample profile as compared with a control profile after contacting the subject with a cancer therapeutic agent may be indicative of lower susceptibility to treatment with an agent capable of inhibiting or reversing EMT or a chemotherapeutic agent.

In some preferred embodiments the one or more biomarker includes at least Axl. In some embodiments the one or more biomarker is Axl. In some embodiments an increase in the activity, expression, or amount of Axl in the sample profile as compared with a control profile after contacting the subject with a cancer therapeutic agent is indicative of higher susceptibility to treatment with an agent capable of inhibiting or reversing EMT or a chemotherapeutic agent.

Subject

A “subject” as referred to herein is any species of animal classified as a mammal, and includes but is not restricted to, domestic and farm animals, primates and humans. In some preferred embodiments the subject is a human of any sex or race. In some embodiments, the human is an adult human.

In some embodiments of this aspect of the disclosure, the subject was selected for treatment with the agent capable of inhibiting or reversing EMT or a chemotherapeutic agent, using a predictive method according to the first aspect of this facet of the disclosure outlined above.

Agents Capable of Inhibiting or Reversing EMT

The present disclosure provides methods for predicting a cancer-related outcome in a subject having, suspected of having, or diagnosed with myelodysplastic syndrome (MDS). A “cancer-related outcome” is a clinical prediction or prognosis associated with cancer.

The agents capable of inhibiting or reversing EMT for use in this MDS facet of the disclosure are defined as described above in the AML facet (see identically titled section above on page 14, line 19 to page 17, line 7).

Assessing Biomarker Activity, Expression, Amount

In some embodiments of the predictive methods described herein, assessing the activity, expression, or amount of one or more biomarker in a subject, or in a sample derived from a subject, comprises: contacting a sample, or an extract from a sample, with at least 1, 2, 3, 4, 5, 6, 7, 8, 9, 10 or more reagents for detecting the activity, expression, or amount of one or more biomarkers, each of which is selective for a particular biomarker; and, detecting and/or quantifying said reagents for detecting.

In some preferred embodiments, assessing the expression or amount of the one or more biomarker comprises determining the level of the one or more biomarker protein or mRNA in the subject or sample derived from the subject. Preferably the expression or amount of the one or more biomarker is assessed by determining the level of protein expression. In particularly preferred embodiments the expression or amount of the one or more biomarker is assessed by determining the level of protein expression in serum.

The term “expression” refers to the transcription of a gene's DNA template to produce the corresponding mRNA and translation of this mRNA to produce the corresponding gene product (i.e., a peptide, polypeptide, or protein) as well as the “expression” of a protein in one or more forms that may have been modified post translation.

In some embodiments determining the level of the one or more biomarker protein or mRNA comprises: contacting a sample, or an extract from a sample, with at least 1, 2, 3, 4, 5, 6, 7, 8, 9, 10 or more specific binding members, each of which selectively binds to a respective biomarker protein or mRNA; and, detecting and/or quantifying formation of complex formed by said specific binding member and said biomarker protein or mRNA. In some embodiments the specific binding member may comprise an antibody molecule or a binding fragment thereof.

A range of suitable techniques for determining protein and mRNA expression levels are well known in the art, for example microarray analysis, Western blotting, and PCR techniques such as QPCR. In some embodiments, determining the expression or amount of the one or more biomarker protein or mRNA may comprise use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.

In some embodiments, the expression or amount of the one or more biomarker is determined as an absolute amount of the respective biomarker protein or mRNA in the sample. In such cases, the respective biomarker protein or mRNA may be quantified in the sample and the amount expressed as a measure of the sample, for example the volume, mass, or total protein content of the sample.

In some embodiments, the expression or amount of the one or more biomarker is determined as a relative amount of the respective biomarker protein or mRNA in the sample, wherein the relative amount is determined relative to a reference protein or mRNA in the sample. Determining the relative amount of biomarker protein or mRNA in this way allows for normalisation to, e.g., account for differences in total protein concentration and to remove bias from sample to sample.

Preferably the reference protein or mRNA is one whose expression or amount does not vary significantly between subjects having cancer and subjects not having cancer, in particular MDS. That is, the reference protein or mRNA is preferably one whose expression or amount is not altered by MDS. Accordingly, the reference protein or mRNA may comprise one or more protein or mRNA which is not one of the biomarkers described herein. When expressing the expression or amount of the one or more biomarker as a relative amount, the absolute amount of the respective biomarker protein or mRNA in the sample may be divided by the absolute amount of the reference protein or mRNA in the sample.

Assessing Biomarker Expression at the RNA Level

Gene expression can be detected at the RNA level. RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland). Typical assay formats utilising ribonucleic acid hybridisation include nuclear run-on assays, RT-PCR, RNase protection assays (Melton et al., Nuc. Acids Res. 12:7035), Northern blotting and In Situ hybridization. Gene expression can also be detected by microarray analysis as described below.

Assessing Biomarker Expression at the Protein Level

Preferably biomarker level is assessed by measuring protein expression. Altered gene or protein expression may also be detected by measuring the polypeptides encoded by the gene. This may be achieved by using molecules which bind to the polypeptides encoded by the biomarker gene. Suitable molecules/agents which bind either directly or indirectly to the polypeptides in order to detect the presence of the protein include naturally occurring molecules such as peptides and proteins, for example antibodies, or they may be synthetic molecules.

Antibodies may be derived from commercial sources or through techniques which are familiar to those skilled in the art. In one embodiment, and where altered expression manifests itself through the expression of alteration of post translationally-modified forms of a protein biomarker, antibodies specific for those different forms may be used. For the purposes of this disclosure, the term “antibody”, unless specified to the contrary, includes whole antibodies, or fragments of whole antibodies which retain their binding activity for a target antigen. Such fragments include Fv, F(ab′) and F(ab′)2 fragments, as well as single chain antibodies (scFv). Furthermore, the antibodies and fragments thereof may be humanised antibodies, for example as described in EP239400A. For example: monoclonal and polyclonal antibodies, recombinant antibodies, proteolytic and recombinant fragments of antibodies (Fab, Fv, scFv, diabodies), single-domain antibodies (VHH, sdAb, nanobodies, IgNAR, VNAR), and proteins unrelated to antibodies, which have been engineered to have antibody-like specific binding. Antibodies may be bound to a solid support and/or packaged into kits in a suitable container along with suitable reagents, controls, instructions and the like.

Arrays

Array technology and the various techniques and applications associated with it are well known in the art. Array technology overcomes the disadvantages with traditional methods in molecular biology, which generally work on a “one gene in one experiment” basis, resulting in low throughput and the inability to appreciate the “whole picture” of gene function. In the context of the products and methods of the present disclosure, array technology can be used, for example, in the analysis of the expression of biomarker proteins or mRNA.

In general, any library or group of samples may be arranged in an orderly manner into an array, by spatially separating the members of the library or group. Examples of suitable libraries for arraying include nucleic acid libraries (including DNA, cDNA, oligonucleotide, etc. libraries), peptide, polypeptide and protein libraries, as well as libraries comprising any molecules, such as ligand libraries, among others. Accordingly, where reference is made to a “library” in this document, unless the context dictates otherwise, such reference should be taken to include reference to a library in the form of an array.

Proteins, polypeptides, etc., may also be immobilised in arrays. For example, antibodies have been used in microarray analysis of the proteome using protein chips (Borrebaeck C A, 2000, Immunol Today 21(8):379-82). Polypeptide arrays are reviewed in, for example, MacBeath and Schreiber, 2000, Science, 289(5485):1760-1763.

Sample

Suitable samples include, but are not limited to, tissue samples such as tissue biopsy, blood, urine, buccal scrapes etc., as well as serum, plasma, or tissue culture supernatant samples. In preferred embodiments the expression or amount of the one or more biomarker is assessed by determining the level of protein expression in the sample. In some preferred embodiments the sample is a blood, serum, or plasma sample. In some particularly preferred embodiments the sample is a serum sample.

When assessing the expression or amount of the one or more biomarker proteins or mRNA in serum or plasma samples derived from a subject, samples are removed and subjected to analytical techniques such as flow cytometry, mass cytometry (CyTOF), ELISA, PET, and SELDI-TOF MS. In some embodiments, the method may comprise extracting RNA from said sample and detecting gene expression by QPCR. In some other embodiments, gene expression may be detected by detecting protein products by, for example, Western Blot.

In some embodiments, the level of expression of the one or more biomarker is assessed by determining the copy number of the gene encoding the one or more biomarker. Copy number (i.e. gene duplication events) may be determined using standard techniques known in the art, e.g. using a DNA chip as described in Jiang et al. (Jiang Q, Ho Y Y, Hao L, Nichols Berrios C, Chakravarti A. Copy number variants in candidate genes are genetic modifiers of Hirschsprung disease. PLoS One. 2011; 6(6)).

Is some preferred embodiments of the predictive methods described herein, the method is performed in vitro or ex vivo.

Methods of Selecting a Subject for Treatment

A third aspect of this facet of the present disclosure relates to methods of selecting subjects having, suspected of having, or diagnosed with myelodysplastic syndrome (MDS) for treatment with an agent capable of inhibiting or reversing EMT or a chemotherapeutic agent. In some embodiments the method comprises: identifying subjects susceptible to treatment with an agent capable of inhibiting or reversing EMT or a chemotherapeutic agent using a predictive method defined in accordance with the above described first and second aspects of this facet of the disclosure; and, selecting thus identified subjects for treatment.

In some embodiments this aspect relates to methods of selecting subjects having, suspected of having, or diagnosed with myelodysplastic syndrome (MDS) for continued treatment with an agent capable of inhibiting or reversing EMT or a chemotherapeutic agent. In some embodiments the method comprises: identifying subjects susceptible to treatment with an agent capable of inhibiting or reversing EMT or a chemotherapeutic agent using a predictive method defined in accordance with the above described second aspect of this facet of the disclosure; and, selecting thus identified subjects for treatment.

In some embodiments of this aspect of the disclosure, the treatment comprises administering to the subject a therapeutically effective amount of an agent capable of inhibiting or reversing EMT or a chemotherapeutic agent as described herein. Accordingly, in some embodiments the treatment comprises an Axl inhibitor, Akt3 inhibitor, Slfn11 inhibitor, or PHGDH inhibitor. In some preferred embodiments the treatment comprises an Axl inhibitor, for example, BGB324/R428/bemcentinib, cabozantinib, TP-0903, foretinib, merestinib, bosutinib, gilteritinib, crizotinib, amuvatinib, sunitinib, sitravatinib, LDC1267, or UNC2025. Other Axl inhibitors include the anti-Axl antibodies described in WO2015/193428, WO2015/193430, WO2016/097370, and WO2016/166296. In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In some other preferred embodiments, the treatment comprises an Akt3 inhibitor. In some embodiments the Akt3 inhibitor is an Akt3 inhibitor disclosed in WO2016/102672.

In some embodiments of this aspect of the disclosure, the treatment is administered as a single agent. In other embodiments, the treatment is administered in combination with a further cancer treatment. Suitable further cancer treatments are outlined in detail above. In some embodiments the further cancer treatment is a pyrimidine analogue, for example azacitidine, capecitabine, cytarabine, edatrexate, floxuridine, fluorouracil, gemcitabine, or troxacitabine, or a cytidine analogue, for example decitabine. In some particularly preferred embodiments the further cancer treatment is cytarabine. In other particularly preferred embodiments the further cancer treatment is decitabine.

Kits

A fourth aspect of this facet of the present disclosure relates to diagnostic kits and test devices. In some embodiments the disclosure provides a diagnostic kit comprising 1, 2, 3, 4, 5, 6, 7, 8, 9, 10 or more reagents for detecting the activity, expression, or amount of one or more of the biomarkers described herein, each of which is selective for a particular biomarker. In some embodiments each of the reagents for detecting is selective for a biomarker selected from the group consisting of: Axl, TFR1, FAS, IL-8, THP, TRAIL-R3, MDC, ALP, TFF3, FABP, vWF, VDBP, Cystatin-B, Uteroglobin, Fibrinogen, EGFR, and/or Leptin.

In other embodiments the disclosure provides a test device comprising 1, 2, 3, 4, 5, 6, 7, 8, 9, 10 or more reagents for detecting the activity, expression, or amount of one or more of the biomarkers described herein, each of which is selective for a particular biomarker. In some embodiments each of the reagents for detecting is selective for a biomarker selected from the group consisting of: Axl, TFR1, FAS, IL-8, THP, TRAIL-R3, MDC, ALP, TFF3, FABP, vWF, VDBP, Cystatin-B, Uteroglobin, Fibrinogen, EGFR, and/or Leptin.

In some embodiments the diagnostic kit or test device comprises 1, 2, 3, 4, 5, 6, 7, 8, 9, 10 or more specific binding members, each of which selectively binds to one of the biomarkers described herein; and, one or more reagents for detecting said one or more specific binding members, or one or more reagents for detecting and/or quantifying formation of a complex formed by said specific binding member and said biomarker. In some embodiments each specific binding member selectively binds to a biomarker selected from the group consisting of: Axl, TFR1, FAS, IL-8, THP, TRAIL-R3, MDC, ALP, TFF3, FABP, vWF, VDBP, Cystatin-B, Uteroglobin, Fibrinogen, EGFR, and/or Leptin.

In some embodiments the diagnostic kit or test device comprises a specific binding member which selectively binds to Axl, and at least 1, 2, 3, 4, 5, 6, 7, 8, 9, 10, or more specific binding members, each of which selectively binds to a biomarker selected from the group consisting of: TFR1, FAS, IL-8, THP, TRAIL-R3, MDC, ALP, TFF3, FABP, vWF, VDBP, Cystatin-B, Uteroglobin, Fibrinogen, EGFR, and/or Leptin.

In some embodiments of this aspect of the disclosure the diagnostic kit or test device comprises a plurality of said specific binding members provided in the form of an array on a substrate or conjugated to a plurality of particles, such as beads or microspheres. The particles may be encoded with a detectable label. In some embodiments the specific binding member comprises an antibody molecule or a binding fragment thereof.

In some embodiments of the diagnostic kit or test device detecting formation of a complex formed by a specific binding member and a biomarker is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.

In some embodiments of the diagnostic kit or test device, the kit or test device comprises no more than 15, 20, 25, 30, 40, or 50 reagents for detecting. In some embodiments of the diagnostic kit or test device, the kit or test device comprises no more than 15, 20, 25, 30, 40, or 50 specific binding members.

The present disclosure also provides use of such diagnostic kits and test devices in methods of predicting a cancer-related outcome in a subject. In preferred embodiments the cancer-related outcome is susceptibility to treatment with an agent capable of inhibiting or reversing EMT or a chemotherapeutic agent as described herein.

In some embodiments the agent capable of inhibiting or reversing EMT or chemotherapeutic agent is an Axl inhibitor, Akt3 inhibitor, Slfn11 inhibitor, or PHGDH inhibitor. In some preferred embodiments the agent capable of inhibiting or reversing EMT or chemotherapeutic agent is an Axl inhibitor, for example BGB324/R428/bemcentinib, cabozantinib, TP-0903, foretinib, merestinib, bosutinib, gilteritinib, crizotinib, amuvatinib, sunitinib, sitravatinib, LDC1267, or UNC2025. Other Axl inhibitors include the anti-Axl antibodies described in WO2015/193428, WO2015/193430, WO2016/097370, and WO2016/166296. In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In some embodiments of the diagnostic kit or test device for use in a predictive method, the subject has, is suspected of having, or has been diagnosed with MDS.

In some embodiments the method may be as defined in accordance with the above described first and second aspects of this facet of the disclosure.

A fifth aspect of this facet of the present disclosure relates to the use of a reagent for detecting the activity, expression, or amount of a biomarker selected from the group consisting of: Axl, TFR1, FAS, IL-8, THP, TRAIL-R3, MDC, ALP, TFF3, FABP, vWF, VDBP, Cystatin-B, Uteroglobin, Fibrinogen, EGFR, and/or Leptin, in a method of predicting a cancer-related outcome in a subject.

A sixth aspect of this facet of the disclosure relates to the use of 1, 2, 3, 4, 5, 6, 7, 8, 9, 10 or more reagents for detecting the activity, expression, or amount a biomarker, each of which is selective for a particular biomarker selected from the group consisting of: Axl, TFR1, FAS, IL-8, THP, TRAIL-R3, MDC, ALP, TFF3, FABP, vWF, VDBP, Cystatin-B, Uteroglobin, Fibrinogen, EGFR, and/or Leptin, in a method of manufacture of a diagnostic kit or test device for use in a method of predicting a cancer-related outcome in a subject. In some embodiments of these uses the reagent for detecting comprises a specific binding member selective for a particular biomarker. The specific binding member may comprise an antibody molecule or a binding fragment thereof.

In some embodiments the method may be as defined in accordance with the above described first and second aspects of this facet of the disclosure.

Uses

A seventh aspect of this facet of the present disclosure relates to the use of one or more of: Axl, TFR1, FAS, IL-8, THP, TRAIL-R3, MDC, ALP, TFF3, FABP, vWF, VDBP, Cystatin-B, Uteroglobin, Fibrinogen, EGFR, and/or Leptin, as a biomarker for determining if a subject is susceptible to treatment with an agent capable of inhibiting or reversing EMT or a chemotherapeutic agent.

In some embodiments the use relates to use of Axl, and at least 1, 2, 3, 4, 5, 6, 7, 8, 9, 10, or more of: TFR1, FAS, IL-8, THP, TRAIL-R3, MDC, ALP, TFF3, FABP, vWF, VDBP, Cystatin-B, Uteroglobin, Fibrinogen, EGFR, and/or Leptin, as a biomarker for determining if a subject is susceptible to treatment with an agent capable of inhibiting or reversing EMT or a chemotherapeutic agent.

In some embodiments the use is as a biomarker in a method as defined in accordance with the above described first and second aspects of this facet of the disclosure.

Methods of Treating a Subject

An eighth aspect of this facet of the present disclosure relates to methods of treating a subject having, suspected of having, or diagnosed with myelodysplastic syndrome (MDS). In some embodiments the method comprises: identifying a subject for treatment using a predictive method defined in accordance with the above described first and second aspects of this facet of the disclosure; and, administering to the subject a therapeutically effective amount of an agent capable of inhibiting or reversing EMT or a chemotherapeutic agent.

Accordingly, in some embodiments, the method comprises: (a) obtaining a sample from the subject; (b) determining if the subject is susceptible to treatment with an agent capable of inhibiting or reversing EMT or a chemotherapeutic agent using a predictive method defined in accordance with the above described first and second aspects of this facet of the disclosure; and, (c) administering to the subject a therapeutically effective amount of an agent capable of inhibiting or reversing EMT or a chemotherapeutic agent.

In some embodiments the method comprises administering a therapeutically effective amount of an agent capable of inhibiting or reversing EMT or chemotherapeutic agent to a subject identified as susceptible to said treatment using a predictive method defined in accordance with the above described first and second aspects of this facet of the disclosure.

In other embodiments the method comprises: (a) administering to a subject a therapeutically effective amount of an agent capable of inhibiting or reversing EMT or a chemotherapeutic agent; and, (b) determining if the subject is susceptible to said treatment using a predictive method defined in accordance with the above described second aspect of this facet of the disclosure. In some embodiments the method further comprises: (c) administering to the subject one or more further therapeutically effective amounts of an agent capable of inhibiting or reversing EMT or a chemotherapeutic agent provided that the subject is identified as susceptible to said treatment.

A ninth aspect of this facet of the present disclosure relates to an agent capable of inhibiting or reversing EMT or chemotherapeutic agent for use in a method of treating a subject having, suspected of having, or diagnosed with myelodysplastic syndrome (MDS). In some embodiments the method of treatment is a method as defined above.

A tenth aspect of this facet of the present disclosure relates to use of an agent capable of inhibiting or reversing EMT or chemotherapeutic agent for use in a method of manufacture of a medicament for use in a method of treating a subject having, suspected of having, or diagnosed with myelodysplastic syndrome (MDS). In some embodiments the method of treatment is a method as defined above.

In some embodiments of these methods, agents for use, or uses, the agent capable of inhibiting or reversing EMT or chemotherapeutic agent comprises an Axl inhibitor, Akt3 inhibitor, Slfn11 inhibitor, or PHGDH inhibitor. In some preferred embodiments the agent capable of inhibiting or reversing EMT or chemotherapeutic agent is an Axl inhibitor, for example BGB324/R428/bemcentinib, cabozantinib, TP-0903, foretinib, merestinib, bosutinib, gilteritinib, crizotinib, amuvatinib, sunitinib, sitravatinib, LDC1267, or UNC2025. Other Axl inhibitors include the anti-Axl antibodies described in WO2015/193428, WO2015/193430, WO2016/097370, and WO2016/166296. In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In some other preferred embodiments, the treatment comprises an Akt3 inhibitor. In some embodiments the Akt3 inhibitor is an Akt3 inhibitor disclosed in WO2016/102672.

In some embodiments of these methods, agents for use, or uses, the treatment is administered as a single agent. In other embodiments, the treatment is administered in combination with a further cancer treatment. Suitable further cancer treatments are outlined in detail above. In some embodiments the further cancer treatment is a pyrimidine analogue, for example azacitidine, capecitabine, cytarabine, edatrexate, floxuridine, fluorouracil, gemcitabine, or troxacitabine, or a cytidine analogue, for example decitabine. In some particularly preferred embodiments the further cancer treatment is cytarabine. In other particularly preferred embodiments the further cancer treatment is decitabine.

Statements of Disclosure

The following numbered statements relate to aspects of this facet of the present disclosure, and form part of the description:
101. A method of predicting a cancer-related outcome in a subject, the method comprising assessing the activity, expression, or amount of one or more biomarker in the subject, or in a sample derived from the subject;
   wherein the one or more biomarker is selected from the group consisting of: Axl, TFR1, FAS, IL-8, THP, TRAIL-R3, MDC, ALP, TFF3, FABP, vWF, VDBP, Cystatin-B, Uteroglobin, Fibrinogen, EGFR, and/or Leptin; and
   wherein the subject has, is suspected of having, or has been diagnosed with myelodysplastic syndrome (MDS).
102. A method according to statement 101, wherein the method comprises:
   assessing the activity, expression, or amount of one or more biomarker in the subject, or in a sample derived from the subject, to obtain a sample profile of the one or more biomarker; and
   making a prediction based on the sample profile of the one or more biomarker.
103. A method according to statement 102, wherein the prediction is made by comparing the sample profile to a control profile.
104. A method according to statement 103, wherein the sample profile is obtained before the subject is contacted with or administered an agent capable of inhibiting or reversing EMT or a chemotherapeutic agent.
105. A method according to statement 104, wherein the control profile is:
   (i) obtained from a population of control subjects having MDS; or
   (ii) obtained from a control subject or population of control subjects having MDS and previously found to lack susceptibility to treatment with an agent capable of inhibiting or reversing EMT or a chemotherapeutic agent (that is, non-responder subjects);
   (iii) a predetermined profile of biomarker expression, activity, or amount values, for example a profile of “average, median, or mean” values or “standard ranges” of values, obtained from a control subject or population of control subjects having MDS and previously found to lack susceptibility to treatment with an agent capable of inhibiting or reversing EMT or a chemotherapeutic agent (that is, non-responder subjects);
   (iv) obtained from a control sample having a known “average, median, or mean” value of biomarkers indicative of a control subject or population of control subjects having MDS and previously found to lack susceptibility to treatment with an agent capable of inhibiting or reversing EMT or a chemotherapeutic agent (that is, non-responder subjects);
   (v) a predetermined profile of biomarker expression, activity, or amount “threshold” values obtained from a control subject or population of control subjects having MDS and previously found to be susceptible to treatment with an agent capable of inhibiting or reversing EMT or a chemotherapeutic agent (that is, responder subjects); or
   (vi) obtained from a control sample having a known “threshold” value of biomarkers indicative of a control subject or population of control subjects having MDS and previously found to be susceptible to treatment with an agent capable of inhibiting or reversing EMT or a chemotherapeutic agent (that is, responder subjects).
106. A method according to statement 105, wherein a higher activity, expression, or amount of one or more biomarker in the sample profile as compared with the control profile is indicative of susceptibility to treatment with an agent capable of inhibiting or reversing EMT or a chemotherapeutic agent.
107. A method according to statement 105, wherein a lower activity, expression, or amount of one or more biomarker in the sample profile as compared with the control profile is indicative of susceptibility to treatment with an agent capable of inhibiting or reversing EMT or a chemotherapeutic agent.
108. A method according to any one of statements 105-107, wherein the one or more biomarker includes at least one of: THP, TRAIL-R3, MDC, ALP, TFF3, FABP, vWF, VDBP, Cystatin-B, Uteroglobin, Fibrinogen, EGFR, and/or Leptin,
   and wherein a higher activity, expression, or amount of one or more of said biomarkers in the sample profile as compared with the control profile is indicative of susceptibility to treatment with an agent capable of inhibiting or reversing EMT or a chemotherapeutic agent.
109. A method according to any one of statements 105-108, wherein the one or more biomarker includes at least one of: Axl, TFR1, FAS, and/or IL-8,
   and wherein a lower activity, expression, or amount of one or more of said biomarkers in the sample profile as compared with the control profile is indicative of susceptibility to treatment with an agent capable of inhibiting or reversing EMT or a chemotherapeutic agent.
110. A method according to any one of statements 105-109, wherein:
   a lower activity, expression, or amount of Axl; and/or
   a lower activity, expression, or amount of TFR1; and/or
   a lower activity, expression, or amount of FAS; and/or
   a lower activity, expression, or amount of IL-8; and/or
   a higher activity, expression, or amount of THP; and/or
   a higher activity, expression, or amount of TRAIL-R3; and/or
   a higher activity, expression, or amount of MDC; and/or
   a higher activity, expression, or amount of ALP; and/or
   a higher activity, expression, or amount of TFF3; and/or
   a higher activity, expression, or amount of FABP; and/or
   a higher activity, expression, or amount of vWF; and/or
   a higher activity, expression, or amount of VDBP; and/or
   a higher activity, expression, or amount of Cystatin-B; and/or
   a higher activity, expression, or amount of Uteroglobin; and/or
   a higher activity, expression, or amount of Fibrinogen; and/or
   a higher activity, expression, or amount of EGFR; and/or
   a higher activity, expression, or amount of Leptin;
   in the sample profile as compared with the control profile is indicative of susceptibility to treatment with an agent capable of inhibiting or reversing EMT or a chemotherapeutic agent.
111. A method according to any one of statements 101-110, wherein the one or more biomarker comprises Axl,
   optionally wherein the one or more biomarker comprises Axl and at least 1, 2, 3, 4, 5, 6, 7, 8, 9, 10, or more further biomarkers selected from: TFR1, FAS, IL-8, THP, TRAIL-R3, MDC, ALP, TFF3, FABP, vWF, VDBP, Cystatin-B, Uteroglobin, Fibrinogen, EGFR, and/or Leptin.
112. A method according to any one of statements 101-111, wherein:
   said Axl biomarker comprises an amino acid sequence having at least 70%, 80%, 90%, 95%, 99% or 100% sequence identity with the full-length of the amino acid sequence disclosed at UniProt accession no: P30530-1 or P30530-2 (entry version 196) or a fragment thereof;
   said Transferrin receptor protein 1 (TFR1) biomarker comprises an amino acid sequence having at least 70%, 80%, 90%, 95%, 99% or 100% sequence identity with the full-length of the amino acid sequence disclosed at UniProt accession no: P02786 (entry version 222) or a fragment thereof;
   said FASLG Receptor (FAS) biomarker comprises an amino acid sequence having at least 70%, 80%, 90%, 95%, 99% or 100% sequence identity with the full-length of the amino acid sequence disclosed at UniProt accession no: P25445-1, P25445-2, P25445-3, P25445-4, P25445-5, P25445-6, or P25445-7 (entry version 227) or a fragment thereof;
   said Interleukin-8 (IL-8) biomarker comprises an amino acid sequence having at least 70%, 80%, 90%, 95%, 99% or 100% sequence identity with the full-length of the amino acid sequence disclosed at UniProt accession no: P10145 (entry version 210) or a fragment thereof;
   said Tamm-Horsfall Urinary Glycoprotein (THP) biomarker comprises an amino acid sequence having at least 70%, 80%, 90%, 95%, 99% or 100% sequence identity with the full-length of the amino acid sequence disclosed at UniProt accession no: P07911-1, P07911-2, P07911-3, P07911-4, or P07911-5 (entry version 182) or a fragment thereof;
   said TNF-Related Apoptosis-Inducing Ligand Receptor 3 (TRAIL-R3) biomarker comprises an amino acid sequence having at least 70%, 80%, 90%, 95%, 99% or 100% sequence identity with the full-length of the amino acid sequence disclosed at UniProt accession no: 014798 (entry version 152) or a fragment thereof;
   said Macrophage-Derived Chemokine (MDC) biomarker comprises an amino acid sequence having at least 70%, 80%, 90%, 95%, 99% or 100% sequence identity with the full-length of the amino acid sequence disclosed at UniProt accession no: 000626 (entry version 153) or a fragment thereof;
   said Antileukoproteinase (ALP) biomarker comprises an amino acid sequence having at least 70%, 80%, 90%, 95%, 99% or 100% sequence identity with the full-length of the amino acid sequence disclosed at UniProt accession no: P03973 (entry version 184) or a fragment thereof;
   said Trefoil Factor 3 (TFF3) biomarker comprises an amino acid sequence having at least 70%, 80%, 90%, 95%, 99% or 100% sequence identity with the full-length of the amino acid sequence disclosed at UniProt accession no: Q07654 (entry version 161) or a fragment thereof;
   said Fatty Acid-Binding Protein, adipocyte (FABP) biomarker comprises an amino acid sequence having at least 70%, 80%, 90%, 95%, 99% or 100% sequence identity with the full-length of the amino acid sequence disclosed at UniProt accession no: P15090 (entry version 183) or a fragment thereof;
   said von Willebrand Factor (vWF) biomarker comprises an amino acid sequence having at least 70%, 80%, 90%, 95%, 99% or 100% sequence identity with the full-length of the amino acid sequence disclosed at UniProt accession no: P04275-1 or P04275-2 (entry version 233) or a fragment thereof;
   said Vitamin D-Binding Protein (VDBP) biomarker comprises an amino acid sequence having at least 70%, 80%, 90%, 95%, 99% or 100% sequence identity with the full-length of the amino acid sequence disclosed at UniProt accession no: P02774-1, P02774-2, or P02774-3 (entry version 201) or a fragment thereof;
   said Cystatin-B biomarker comprises an amino acid sequence having at least 70%, 80%, 90%, 95%, 99% or 100% sequence identity with the full-length of the amino acid sequence disclosed at UniProt accession no: P04080 (entry version 201) or a fragment thereof;
   said Uteroglobin biomarker comprises an amino acid sequence having at least 70%, 80%, 90%, 95%, 99% or 100% sequence identity with the full-length of the amino acid sequence disclosed at UniProt accession no: P11684 (entry version 163) or a fragment thereof;
   said Fibrinogen biomarker comprises an amino acid sequence having at least 70%, 80%, 90%, 95%, 99% or 100% sequence identity with the full-length of the amino acid sequence disclosed at UniProt accession no: P02671-1 or P02671-2 (entry version 227) or a fragment thereof;
   said Epidermal Growth Factor Receptor (EGFR) biomarker comprises an amino acid sequence having at least 70%, 80%, 90%, 95%, 99% or 100% sequence identity with the full-length of the amino acid sequence disclosed at UniProt accession no: Q504U8 (entry version 138) or a fragment thereof; and/or
   said Leptin biomarker comprises an amino acid sequence having at least 70%, 80%, 90%, 95%, 99% or 100% sequence identity with the full-length of the amino acid sequence disclosed at UniProt accession no: P41159 (entry version 176) or a fragment thereof;
   or a nucleic acid (either DNA or RNA) having at least 70%, 80%, 90%, 95%, 99% or 100% sequence identity with the full-length nucleic acid sequence encoding said amino acid sequences.
113. A method according to any one of statement 101-112, wherein the method comprises determining the amount of at least 2, 3, 4, 5, 6, 7, 8, 9, 10, 11, 12, 13, 14, 15, 20 of said biomarkers in the subject, or sample derived from the subject.
114. A method according to any one of statements 104-113, wherein assessing the activity, expression, or amount of one or more biomarker in the subject, or in a sample derived from the subject, comprises:
   contacting a sample, or an extract from the sample, with at least 1, 2, 3, 4, 5, 6, 7, 8, 9, 10 or more reagents for detecting the activity, expression, or amount of one or more biomarkers, each of which is selective for a particular biomarker; and
   detecting and/or quantifying said reagents for detecting.
115. A method according to statement 114, wherein assessing the expression or amount of one or more biomarker in the subject, or in a sample derived from the subject, comprises:
   contacting a sample, or an extract from the sample, with at least 1, 2, 3, 4, 5, 6, 7, 8, 9, 10 or more specific binding members, each of which selectively binds to a respective biomarker protein or mRNA; and
   detecting and/or quantifying formation of complex formed by said specific binding member and said biomarker protein or mRNA.
116. A method according to statement 115 wherein the specific binding member comprises an antibody molecule or binding fragment thereof.
117. A method according to statement 115 or 116, wherein detecting formation of a complex formed by the specific binding member and the biomarker protein or mRNA is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.
118. A method according to any one of statements 115-117 wherein the expression or amount of the one or more biomarker is determined as the absolute amount of the respective biomarker protein or mRNA in the sample.
119. A method according to any one of statements 115-117 wherein the expression or amount of the one or more biomarker is determined as a relative amount of the respective biomarker protein or mRNA in the sample,
   optionally wherein the relative amount is determined relative to a reference protein or mRNA in the sample, the expression of which is not altered by MDS.
120. A method according to any one of statements 101-119, wherein the cancer-related outcome is susceptibility to treatment with an agent capable of inhibiting or reversing EMT or a chemotherapeutic agent.
121. A method according to statement 120, wherein the agent capable of inhibiting or reversing EMT or chemotherapeutic agent is an Axl inhibitor, Akt3 inhibitor, Slfn11 inhibitor, or PHGDH inhibitor.
122. A method according to statement 121, wherein the agent capable of inhibiting or reversing EMT or chemotherapeutic agent is an Axl inhibitor.
123. A method according to statement 122, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
124. A method according to statement 123, wherein the Axl inhibitor is BGB324/R428/bemcentinib.
125. A method according to statement 123, wherein the Axl inhibitor is an anti-Axl antibody described in WO2015/193428, WO2015/193430, WO2016/097370, or WO2016/166296.
126. A method according to statement 121, wherein the agent capable of inhibiting or reversing EMT or chemotherapeutic agent is an Akt3 inhibitor.
127. A method according to statement 126, wherein the Akt3 inhibitor is an Akt3 inhibitor disclosed in WO2016/102672.
128. A method according to any one of statements 120-127, wherein the agent capable of inhibiting or reversing EMT is administered as a single agent.
129. A method according to any one of statements 120-127, wherein the agent capable of inhibiting or reversing EMT is administered in combination with a further cancer treatment.
130. A method according to statement 129, wherein the further cancer treatment is selected from:
   (i) alkylating agents, including alkyl sulfonates such as busulfan;
   (ii) nitrogen mustards such as chlorambucil, cyclophosphamide, estramustine, ifosfamide, mechlorethamine, melphalan, and uramustine;
   (iii) ethyleneimine derivatives such as thiotepa;
   (iv) nitrosoureas such as carmustine, lomustine, and streptozocin;
   (v) triazenes such as dacarbazine, procarbazine, and temozolamide;
   (vi) platinum compounds such as cisplatin, carboplatin, oxaliplatin, satraplatin, and picoplatin onnaplatin, tetraplatin, sprioplatin, iproplatin, chloro(diethylenediamino)-platinum (II) chloride, dichloro(ethylenediamino)-platinum (II), diamino(2-ethylmalonato)platinum (II), (1,2-diaminocyclohexane)malonatoplatinum (II), (4-carboxyphthalo)-(1,2-diaminocyclohexane)platinum (II), (1,2-diaminocyclohexane)-(isocitrato)platinum (II), and (1,2-diaminocyclohexane)-cis-(pyruvato)platinum (II);
   (vii) antimetabolites, including antifolates such as methotrexate, permetrexed, raltitrexed, and trimetrexate;
   (viii) pyrimidine analogues such as azacitidine, capecitabine, cytarabine, edatrexate, floxuridine, fluorouracil, gemcitabine, and troxacitabine;
   (ix) purine analogues such as cladribine, chlorodeoxyadenosine, clofarabine, fludarabine, mercaptopurine, pentostatin, and thioguanine;
   (x) natural products, including antitumor antibiotics such as bleomycin, dactinomycin, mithramycin, mitomycin, mitoxantrone, porfiromycin;
   (xi) anthracyclines such as daunorubicin, doxorubicin, epirubicin, idarubicin, and valrubicin;
   (xii) mitotic inhibitors such as the vinca alkaloids vinblastine, vinvesir, vincristine, vindesine, and vinorelbine;
   (xiii) enzymes such as L-asparaginase and PEG-L-asparaginase;
   (xiv) microtubule polymer stabilizers such as the taxanes paclitaxel and docetaxel;
   (xv) topoisomerase I inhibitors such as the camptothecins irinotecan and topotecan;
   (xvi) topoisomerase II inhibitors such as podophyllotoxin, amsacrine, etoposide, teniposide, losoxantrone and actinomycin;
   (xvii) hormones and hormone antagonists, including
      androgens such as fluoxymesterone and testolactone;
      antiandrogens such as bicalutamide, cyproterone, flutamide, and nilutamide;
      corticosteroids such as dexamethasone and prednisone;
      aromatase inhibitors such as aminoglutethimide, anastrozole, exemestane, formestane, and letrozole;
      estrogens such as diethylstilbestrol;
      antiestrogens such as fulvestrant, raloxifene, tamoxifen, and toremifine;
      luteinising hormone-releasing hormone (LHRH) agonists and antagonists such as abarelix, buserelin, goserelin, leuprolide, histrelin, desorelin, nafarelin acetate and triptorelin;
      progestins such as medroxyprogesterone acetate and megestrol acetate; and
      thyroid hormones such as levothyroxine and liothyronine;
   (xviii) PKB pathway inhibitors, including perifosine, enzastaurin hydrochloride, and triciribine;
   (xix) P13K inhibitors such as semaphore and SF1126, and MTOR inhibitors such as rapamycin and analogues;
   (xx) CDK inhibitors, including seliciclib, alvocidib, and 7-hydroxystaurosporine;
   (xxi) COX-2 inhibitors, including celecoxib;
   (xxii) HDAC inhibitiors, including trichostatin A, suberoylanilide hydroxamic acid, and chlamydocin;
   (xxiii) DNA methylase inhibitors, including temozolomide, and miscellaneous agents, including altretamine, arsenic trioxide, thalidomide, lenalidomide, gallium nitrate, levamisole, mitotane, hydroxyurea, octreotide, procarbazine, suramin, photodynamic compounds such as methoxsalen and sodium porfimer;
   (xxiv) proteasome inhibitors such as bortezomib;
   (xxv) molecular targeted therapy agents including: functional therapeutic agents, including gene therapy agents and antisense therapy agents;
   (xxvi) tyrosine kinase inhibitors such as erlotinib hydrochloride, gefitinib, imatinib mesylate, and semaxanib;
   (xxvii) Raf inhibitors such as sorafenib, and gene expression modulators such as the retinoids and rexinoids, for example adapalene, bexarotene, trans-retinoic acid, 9-cis-retinoic acid, and N-(4-hydroxyphenyl)retinamide;
   (xxviii) phenotype-directed therapy agents, including monoclonal antibodies such as pembrolizumab, alemtuzumab, bevacizumab, cetuximab, ibritumomab tiuxetan, rituximab, and trastuzumab, immunotoxins such as gemtuzumab ozogamicin, radioimmunoconjugates such as I-tositumobab;
   (xxix) cancer vaccines;
   (xxx) biologic therapy agents including: interferons such as interferon-[alpha]2a and interferon-[alpha]2b, and interleukins such as aldesleukin, denileukin diftitox, and oprelvekin;
   (xxxi) anticancer therapies involving the use of protective or adjunctive agents, including: cytoprotective agents such as amifostine, and dexrazoxane, phosphonates such as pamidronate and zoledronic acid, and stimulating factors such as epoetin, darbeopetin, filgrastim, PEG-filgrastim, and sargramostim;
   (xxxii) Axl inhibitors such as 1-(6,7-dihydro-5H-benzo[6,7]cyclohepta[1,2-c]pyridazin-3-yl)-N3-((7-(S)-pyrrolidin-1-yl)-6,7,8,9-tetrahydro-5H-benzo[7]annulene-2-yl)-1H-1,2,4-triazole-3,5-diamine; and/or
   (xxxiii) further combination chemotherapeutic regimens, such as combinations of carboplatin/paclitaxel, capecitabine/docetaxel, fluorauracil/levamisole, fluorauracil/leucovorin, methotrexate/leucovorin, and trastuzumab/paclitaxel, alone or in further combination with carboplatin.
131. A method according to statement 129, wherein the further cancer treatment is a pyrimidine analogue, for example azacitidine, capecitabine, cytarabine, edatrexate, floxuridine, fluorouracil, gemcitabine, or troxacitabine, or a cytidine analogue, for example decitabine.
132. A method according to statement 131, wherein the further cancer treatment is cytarabine.
133. A method according to statement 131, wherein the further cancer treatment is decitabine.
134. A method according to any one of statements 101-133, wherein the subject is mammalian.
135. A method according to statement 134, wherein the subject is human.
136. A method according to any one of statements 101-135, wherein the predictive method is performed before the subject receives a treatment or course of treatment for MDS.
137. A method according to any one of statement 101-136, wherein the predictive method is performed during or after a treatment or course of treatment for MDS.
138. A method according to any one of statements 101-135 or 137, wherein the subject has previously been treated with an agent capable of inhibiting or reversing EMT or a chemotherapeutic agent.
139. A method according to statement 138, wherein the subject has previously been treated with an agent selected from: a PHGDH inhibitor, a Slfn11 inhibitor, an Axl inhibitor, or an Akt3 inhibitor.
140. A method according to any one of statements 138-139, wherein the subject has previously been treated with an agent that is different to the agent capable of inhibiting or reversing EMT or chemotherapeutic agent for which the subject's susceptibility is being predicted.
141. A method according to any one of statements 101-136, wherein the subject has not previously been treated with an agent capable of inhibiting or reversing EMT or a chemotherapeutic agent.
142. A method according to statement 141, wherein the subject has not previously been treated with an Axl inhibitor.
143. A method according to statement 141, wherein the subject has not previously been treated with an Akt3 inhibitor.
144. A method according to any one of statements 101-143, wherein the sample is selected from: blood, serum, plasma, tissue biopsy, culture supernatant, or urine.
145. A method according to statement 144, wherein the sample is a blood, serum, or plasma sample.
146. A method according to statement 145, wherein the sample is a serum sample.
147. A method according to any one of statements 104-146, wherein the method further comprises a subsequent step of assessing the activity, expression, or amount of one or more biomarker in the subject, in which a sample profile is obtained after contacting the subject with an agent capable of inhibiting or reversing EMT or a chemotherapeutic agent.
148. A method according to statement 103, wherein the sample profile is obtained after contacting the subject with an agent capable of inhibiting or reversing EMT or a chemotherapeutic agent.
149. A method according to statement 147 or 148, wherein the control profile is obtained from the same subject prior to contacting said subject with the agent capable of inhibiting or reversing EMT or a chemotherapeutic agent.
150. A method according to statement 149, wherein an increase in the activity, expression, or amount of one or more biomarker in the sample profile as compared with the control profile is indicative of higher susceptibility to treatment with an agent capable of inhibiting or reversing EMT or a chemotherapeutic agent.
151. A method according to statement 149, wherein a decrease in the activity, expression, or amount of one or more biomarker in the sample profile as compared with the control profile is indicative of higher susceptibility to treatment with an agent capable of inhibiting or reversing EMT or a chemotherapeutic agent.
152. A method according to statement 149, wherein an increase in the activity, expression, or amount of one or more biomarker in the sample profile as compared with the control profile is indicative of lower susceptibility to treatment with an agent capable of inhibiting or reversing EMT or a chemotherapeutic agent.
153. A method according to statement 149, wherein a decrease in the activity, expression, or amount of one or more biomarker in the sample profile as compared with the control profile is indicative of lower susceptibility to treatment with an agent capable of inhibiting or reversing EMT or a chemotherapeutic agent.
154. A method according to statement 150, wherein the one or more biomarker includes at least Axl.
155. A method according to any one of statements 147-154, wherein assessing the activity, expression, or amount of one or more biomarker in the subject, or in a sample derived from the subject, comprises:
   contacting a sample, or an extract from the sample, with at least 1, 2, 3, 4, 5, 6, 7, 8, 9, 10 or more reagents for detecting the activity, expression, or amount of one or more biomarkers, each of which is selective for a particular biomarker; and
   detecting and/or quantifying said reagents for detecting.
156. A method according to statement 155, wherein assessing the expression or amount of one or more biomarker in the subject, or in a sample derived from the subject, comprises:
   contacting a sample, or an extract from the sample, with at least 1, 2, 3, 4, 5, 6, 7, 8, 9, 10 or more specific binding members, each of which selectively binds to a respective biomarker protein or mRNA; and
   detecting and/or quantifying formation of a complex formed by said specific binding member and said biomarker protein or mRNA.
157. A method according to statement 156, wherein the specific binding member comprises an antibody molecule or binding fragment thereof.
158. A method according to statement 156 or 157, wherein detecting formation of a complex formed by the specific binding member and the biomarker protein or mRNA is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.
159. A method according to any one of statements 156-158 wherein the expression or amount of the one or more biomarker is determined as the absolute amount of the respective biomarker protein or mRNA in the sample.
160. A method according to any one of statements 156-158 wherein the expression or amount of the one or more biomarker is determined as a relative amount of the respective biomarker protein or mRNA in the sample,
   optionally wherein the relative amount is determined relative to a reference protein or mRNA in the sample, the expression of which is not altered by MDS.
161. A method according to any one of statements 148-160, wherein the agent capable of inhibiting or reversing EMT or chemotherapeutic agent is an Axl inhibitor, Akt3 inhibitor, Slfn11 inhibitor, or PHGDH inhibitor.
162. A method according to statement 161, wherein the agent capable of inhibiting or reversing EMT or chemotherapeutic agent is an Axl inhibitor.
163. A method according to statement 162, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
164. A method according to statement 163, wherein the Axl inhibitor is BGB324/R428/bemcentinib.
165. A method according to statement 163, wherein the Axl inhibitor is an anti-Axl antibody described in WO2015/193428, WO2015/193430, WO2016/097370, or WO2016/166296.
166. A method according to statement 161, wherein the agent capable of inhibiting or reversing EMT or chemotherapeutic agent is an Akt3 inhibitor.
167. A method according to statement 166, wherein the Akt3 inhibitor is an Akt3 inhibitor disclosed in WO2016/102672.
168. A method according to any one of statements 161-167, wherein the agent capable of inhibiting or reversing EMT is administered as a single agent.
169. A method according to any one of statements 161-167, wherein the agent capable of inhibiting or reversing EMT is administered in combination with a further cancer treatment.
170. A method according to statement 169, wherein the further cancer treatment is selected from the further cancer treatments listed in statement 130.
171. A method according to statement 169, wherein the further cancer treatment is a pyrimidine analogue, for example azacitidine, capecitabine, cytarabine, edatrexate, floxuridine, fluorouracil, gemcitabine, or troxacitabine, or a cytidine analogue, for example decitabine.
172. A method according to statement 171, wherein the further cancer treatment is cytarabine. 173. A method according to statement 171, wherein the further cancer treatment is decitabine.
174. A method according to any one of statements 148-173, wherein the subject is mammalian.
175. A method according to statement 174, wherein the subject is human.
176. A method according to any one of statements 148-175, wherein the subject was selected for treatment with the agent capable of inhibiting or reversing EMT or a chemotherapeutic agent, using a predictive method according to any one of statements 101-147.
177. A method according to any one of statements 148-176, wherein the sample is selected from: blood, serum, plasma, tissue biopsy, culture supernatant, or urine.
178. A method according to statement 177, wherein the sample is a blood, serum, or plasma sample.
179. A method according to statement 178, wherein the sample is a serum sample.
180. A method according to any one of statements 1-179, wherein the method is performed in vitro or ex vivo.
201. A method of selecting subjects having, suspected of having, or diagnosed with myelodysplastic syndrome (MDS) for treatment with an agent capable of inhibiting or reversing EMT or a chemotherapeutic agent, the method comprising:
   identifying subjects susceptible to treatment with an agent capable of inhibiting or reversing EMT or a chemotherapeutic agent using a method according to any one of statements 101-180; and
   selecting thus identified subjects for treatment.
202. A method of selecting subjects having, suspected of having, or diagnosed with myelodysplastic syndrome (MDS) for continued treatment with an agent capable of inhibiting or reversing EMT or a chemotherapeutic agent, the method comprising:
   identifying subjects susceptible to treatment with an agent capable of inhibiting or reversing EMT or a chemotherapeutic agent using a method according to any one of statements 148-180; and
   selecting thus identified subjects for treatment.
203. A method according to any one of statements 201-202, wherein treatment comprises administering to the subject a therapeutically effective amount of an agent capable of inhibiting or reversing EMT or a chemotherapeutic agent.
204. A method according to statement 203, wherein the treatment comprises an Axl inhibitor, Akt3 inhibitor, Slfn11 inhibitor, or PHGDH inhibitor.
205. A method according to statement 204, wherein the treatment comprises an Axl inhibitor.
206. A method according to statement 205, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
207. A method according to statement 206, wherein the Axl inhibitor is BGB324/R428/bemcentinib.
208. A method according to statement 206, wherein the Axl inhibitor is an anti-Axl antibody described in WO2015/193428, WO2015/193430, WO2016/097370, or WO2016/166296.
209. A method according to statement 204, wherein the treatment comprises an Akt3 inhibitor.
210. A method according to statement 209, wherein the Akt3 inhibitor is an Akt3 inhibitor disclosed in WO2016/102672.
211. A method according to any one of statements 203-210, wherein the treatment is administered as a single agent.
212. A method according to any one of statements 203-211, wherein the treatment is administered in combination with a further cancer treatment.
213. A method according to statement 212, wherein the further cancer treatment is selected from the further cancer treatments listed in statement 130.
214. A method according to statement 212, wherein the further cancer treatment is a pyrimidine analogue, for example azacitidine, capecitabine, cytarabine, edatrexate, floxuridine, fluorouracil, gemcitabine, or troxacitabine, or a cytidine analogue, for example decitabine.
215. A method according to statement 214, wherein the further cancer treatment is cytarabine.
216. A method according to statement 214, wherein the further cancer treatment is decitabine.
301. A diagnostic kit comprising 1, 2, 3, 4, 5, 6, 7, 8, 9, 10 or more reagents for detecting the activity, expression, or amount of one or more biomarkers, each of which is selective for a particular biomarker selected from the group consisting of: Axl, TFR1, FAS, IL-8, THP, TRAIL-R3, MDC, ALP, TFF3, FABP, vWF, VDBP, Cystatin-B, Uteroglobin, Fibrinogen, EGFR, and/or Leptin.
302. A test device comprising 1, 2, 3, 4, 5, 6, 7, 8, 9, 10 or more reagents for detecting the activity, expression, or amount of one or more biomarkers, each of which is selective for a particular biomarker selected from the group consisting of: Axl, TFR1, FAS, IL-8, THP, TRAIL-R3, MDC, ALP, TFF3, FABP, vWF, VDBP, Cystatin-B, Uteroglobin, Fibrinogen, EGFR, and/or Leptin.
303. A diagnostic kit or test device according to any one of statements 301 or 302, comprising:
   1, 2, 3, 4, 5, 6, 7, 8, 9, 10 or more specific binding members, each of which selectively binds to a biomarker selected from the group consisting of: Axl, TFR1, FAS, IL-8, THP, TRAIL-R3, MDC, ALP, TFF3, FABP, vWF, VDBP, Cystatin-B, Uteroglobin, Fibrinogen, EGFR, and/or Leptin; and
   one or more reagents for detecting said one or more specific binding members or one or more reagents for detecting and/or quantifying formation of a complex formed by said specific binding member and said biomarker.
304. A diagnostic kit or test device according to statement 303, wherein the specific binding member comprises an antibody molecule or binding fragment thereof.
305. A diagnostic kit or test device according to statement 303 or 304, wherein detecting formation of a complex formed by the specific binding member and the biomarker is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.
306. A diagnostic kit or test device according to any one of statements 301-305, wherein the kit or test device comprises no more than 15, 20, 25, 30, 40, or 50 reagents for detecting.
307. A diagnostic kit or test device according to any one of statements 301-306 for use in a method of predicting a cancer-related outcome in a subject.
308. A diagnostic kit for use, or test device for use, according to statement 307, wherein the cancer-related outcome is susceptibility to treatment with an agent capable of inhibiting or reversing EMT or a chemotherapeutic agent.
309. A diagnostic kit for use, or test device for use, according to statement 308, wherein the agent capable of inhibiting or reversing EMT or chemotherapeutic agent is an Axl inhibitor, Akt3 inhibitor, Slfn11 inhibitor, or PHGDH inhibitor.
310. A diagnostic kit for use, or test device for use, according to statement 308, wherein the agent capable of inhibiting or reversing EMT or chemotherapeutic agent is an Axl inhibitor.
311. A diagnostic kit for use, or test device for use, according to statement 310, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
312. A diagnostic kit for use, or test device for use, according to statement 311, wherein the Axl inhibitor is BGB324/R428/bemcentinib.
313. A diagnostic kit for use, or test device for use, according to statement 311, wherein the Axl inhibitor is an anti-Axl antibody described in WO2015/193428, WO2015/193430, WO2016/097370, or WO2016/166296.
314. A diagnostic kit for use, or test device for use, according to statement 308, wherein the agent capable of inhibiting or reversing EMT or chemotherapeutic agent is an Akt3 inhibitor.
315. A diagnostic kit for use, or test device for use, according to any one of statements 307-310, wherein the subject has, is suspected of having, or has been diagnosed with myelodysplastic syndrome (MDS).
316. A diagnostic kit for use, or test device for use, according to any one of statements 307-315, wherein the method is a method according to any one of statements 101-180.
317. Use of a reagent for detecting the activity, expression, or amount of a biomarker selected from the group consisting of: Axl, TFR1, FAS, IL-8, THP, TRAIL-R3, MDC, ALP, TFF3, FABP, vWF, VDBP, Cystatin-B, Uteroglobin, Fibrinogen, EGFR, and/or Leptin, in a method of predicting a cancer-related outcome in a subject.
318. Use of 1, 2, 3, 4, 5, 6, 7, 8, 9, 10 or more reagents for detecting the activity, expression, or amount a biomarker, each of which is selective for a particular biomarker selected from the group consisting of: Axl, TFR1, FAS, IL-8, THP, TRAIL-R3, MDC, ALP, TFF3, FABP, vWF, VDBP, Cystatin-B, Uteroglobin, Fibrinogen, EGFR, and/or Leptin, in a method of manufacture of a diagnostic kit or test device for use in a method of predicting a cancer-related outcome in a subject.
319. A use according to statement 317 or 318, wherein the reagent for detecting comprises a specific binding member selective for a particular biomarker.
320. A use according to statement 317 or 318, wherein the method of predicting a cancer-related outcome in a subject is a method according to any one of statements 101-180.
401. Use of one or more of: Axl, TFR1, FAS, IL-8, THP, TRAIL-R3, MDC, ALP, TFF3, FABP, vWF, VDBP, Cystatin-B, Uteroglobin, Fibrinogen, EGFR, and/or Leptin, as a biomarker for determining if a subject is susceptible to treatment with an agent capable of inhibiting or reversing EMT or a chemotherapeutic agent.
402. A use according to statement 401, wherein:
   (i) a higher activity, expression, or amount of one or more of said biomarkers in the subject, or sample derived from the subject, compared with a control profile for the respective biomarker is indicative of susceptibility to treatment with an agent capable of inhibiting or reversing EMT or a chemotherapeutic agent; or
   (ii) a lower activity, expression, or amount of one or more of said biomarkers in the subject, or sample derived from the subject, compared with a control profile for the respective biomarker is indicative of susceptibility to treatment with an agent capable of inhibiting or reversing EMT or a chemotherapeutic agent.
403. A use according to statement 401, wherein:
   (i) an increase in the activity, expression, or amount of one or more biomarker relative to a control profile for the respective biomarker after contacting the subject with a cancer therapeutic agent is indicative of increased susceptibility to treatment with an agent capable of inhibiting or reversing EMT or a chemotherapeutic agent; or
   (ii) a decrease in the activity, expression, or amount of one or more biomarker relative to a control profile for the respective biomarker after contacting the subject with a cancer therapeutic agent is indicative of increased susceptibility to treatment with an agent capable of inhibiting or reversing EMT or a chemotherapeutic agent.
404. A use according to any one of statements 401-403, wherein the use is as a biomarker in a method according to any one of statements 101-180
501. A method of treating a subject having, suspected of having, or diagnosed with myelodysplastic syndrome (MDS), the method comprising:
   identifying a subject susceptible to treatment using a method according to any one of statements 101-180; and
   administering to the subject a therapeutically effective amount of an agent capable of inhibiting or reversing EMT or a chemotherapeutic agent.
502. A method of treating a subject having, suspected of having, or diagnosed with myelodysplastic syndrome (MDS), the method comprising:
   (a) obtaining a sample from the subject;
   (b) determining if the subject is susceptible to treatment with an agent capable of inhibiting or reversing EMT or a chemotherapeutic agent using a method according to any one of statements 101-180;
   (c) administering to the subject a therapeutically effective amount of an agent capable of inhibiting or reversing EMT or a chemotherapeutic agent.
503. A method of treating a subject having, suspected of having, or diagnosed with myelodysplastic syndrome (MDS), the method comprising administering a therapeutically effective amount of an agent capable of inhibiting or reversing EMT or chemotherapeutic agent to a subject identified as susceptible to said treatment using a method according to any one of statements 101-180.
504. A method of treating a subject having, suspected of having, or diagnosed with myelodysplastic syndrome (MDS), the method comprising:
   administering to the subject a therapeutically effective amount of an agent capable of inhibiting or reversing EMT or a chemotherapeutic agent; and
   determining if the subject is susceptible to said treatment using a method according to any one of statements 148-180.
505. A method according to statement 504, wherein the method comprises administering to the subject one or more further therapeutically effective amounts of an agent capable of inhibiting or reversing EMT or a chemotherapeutic agent provided that the subject is identified as susceptible to said treatment.
506. An agent capable of inhibiting or reversing EMT or chemotherapeutic agent for use in a method of treating a subject having, suspected of having, or diagnosed with myelodysplastic syndrome (MDS),
   wherein the method is a method according to any one of statements 501-505.
507. Use of an agent capable of inhibiting or reversing EMT or chemotherapeutic agent for use in a method of manufacture of a medicament for use in a method of treating a subject having, suspected of having, or diagnosed with myelodysplastic syndrome (MDS),
   wherein the method of treatment is a method according to any one of statements 501-505.
508. A method, agent for use, or use according to any one of statements 501-507, wherein the agent capable of inhibiting or reversing EMT or chemotherapeutic agent comprises an Axl inhibitor, Akt3 inhibitor, Slfn11 inhibitor, or PHGDH inhibitor.
509. A method, agent for use, or use according to statement 508, wherein the agent capable of inhibiting or reversing EMT or chemotherapeutic agent comprises an Axl inhibitor.
510. A method, agent for use, or use according to statement 509, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
511. A method, agent for use, or use according to statement 510, wherein the Axl inhibitor is BGB324/R428/bemcentinib.
512. A method, agent for use, or use according to statement 510, wherein the Axl inhibitor is an anti-Axl antibody described in WO2015/193428, WO2015/193430, WO2016/097370, or WO2016/166296.
512. A method, agent for use, or use according to statement 508, wherein the agent capable of inhibiting or reversing EMT or chemotherapeutic agent comprises an Akt3 inhibitor.
513. A method, agent for use, or use according to statement 512, wherein the Akt3 inhibitor is an Akt3 inhibitor disclosed in WO2016/102672.
514. A method, agent for use, or use according to any one of statements 501-513, wherein the agent capable of inhibiting or reversing EMT or chemotherapeutic agent is administered as a single agent.
515. A method, agent for use, or use according to any one of statements 501-513, wherein the agent capable of inhibiting or reversing EMT or chemotherapeutic agent is administered in combination with a further cancer treatment.
516. A method, agent for use, or use according to statement 515, wherein the further cancer treatment is selected from the further cancer treatments listed in statement 130.
517. A method, agent for use, or use according to statement 515, wherein the further cancer treatment is a pyrimidine analogue, for example azacitidine, capecitabine, cytarabine, edatrexate, floxuridine, fluorouracil, gemcitabine, or troxacitabine, or a cytidine analogue, for example decitabine.
518. A method, agent for use, or use according to statement 517, wherein the further cancer treatment is cytarabine.
519. A method, agent for use, or use according to statement 517, wherein the further cancer treatment is decitabine.

Facet 3: Acute Myelocytic Leukemia (AML) & Myelodysplastic Syndrome (MDS)

Biomarkers

The term “marker” or “biomarker” is used herein to refer to a gene or protein whose expression in a subject or sample derived from a subject is altered or modulated, for example, up or down regulated, in cancer. Where the biomarker is a protein, modulation or alteration of expression encompasses modulation through different post-translational modifications.

In accordance with any aspect of this facet of the present disclosure the biomarkers described herein may be defined as follows, wherein:
an Axl biomarker comprises an amino acid sequence having at least 70%, 80%, 90%, 95%, 99% or 100% sequence identity with the full-length of the amino acid sequence disclosed at UniProt accession no: P30530-1 or P30530-2 (entry version 196) or a fragment thereof;
a Fibroblast Growth Factor 21 (FGF-21) biomarker comprises an amino acid sequence having at least 70%, 80%, 90%, 95%, 99% or 100% sequence identity with the full-length of the amino acid sequence disclosed at UniProt accession no: Q9NSA1 (entry version 145) or a fragment thereof;
a Matrix Metalloproteinase-10 (MMP-10) biomarker comprises an amino acid sequence having at least 70%, 80%, 90%, 95%, 99% or 100% sequence identity with the full-length of the amino acid sequence disclosed at UniProt accession no: P09238 (entry version 185) or a fragment thereof;
a Transferrin receptor protein 1 (TFR1) biomarker comprises an amino acid sequence having at least 70%, 80%, 90%, 95%, 99% or 100% sequence identity with the full-length of the amino acid sequence disclosed at UniProt accession no: P02786 (entry version 222) or a fragment thereof;
a Tamm-Horsfall Urinary Glycoprotein (THP) biomarker comprises an amino acid sequence having at least 70%, 80%, 90%, 95%, 99% or 100% sequence identity with the full-length of the amino acid sequence disclosed at UniProt accession no: P07911-1, P07911-2, P07911-3, P07911-4, or P07911-5 (entry version 182) or a fragment thereof;
a Hemopexin biomarker comprises an amino acid sequence having at least 70%, 80%, 90%, 95%, 99% or 100% sequence identity with the full-length of the amino acid sequence disclosed at UniProt accession no: P02790 (entry version 179) or a fragment thereof;
a Haptoglobin biomarker comprises an amino acid sequence having at least 70%, 80%, 90%, 95%, 99% or 100% sequence identity with the full-length of the amino acid sequence disclosed at UniProt accession no: P00738-1 or P00738-2 (entry version 200) or a fragment thereof; and/or
an Immunoglobulin M (IgM) biomarker comprises an amino acid sequence having at least 70%, 80%, 90%, 95%, 99% or 100% sequence identity with the full-length of the amino acid sequence disclosed at UniProt accession no: P01871-1 or P01871-2 (entry version 180) or a fragment thereof.

The biomarkers described herein may also be defined as a nucleic acid (either DNA or RNA) having at least 70%, 80%, 90%, 95%, 99% or 100% sequence identity with the full-length nucleic acid sequence encoding any of the above amino acid sequences.

Identity may be as defined using sequence comparisons made using FASTA and FASTP (see Pearson & Lipman, 1988. Methods in Enzymology 183: 63-98). Parameters are preferably set, using the default matrix, as follows: Gapopen (penalty for the first residue in a gap): −12 for proteins/-16 for DNA; Gapext (penalty for additional residues in a gap): −2 for proteins/-4 for DNA; KTUP word length: 2 for proteins/6 for DNA.

Pre-Treatment Predictive Biomarkers and Methods

By analysing patient data obtained from clinical trial BGBC003 (NCT02488408), the authors have discovered that, prior to treatment of subjects having AML or MDS, certain of the biomarkers described herein are present in the serum of “responder” subjects at higher levels than in the serum of “non-responder” subjects—see Example 1. Similarly, prior to treatment of the subject certain of the biomarkers described herein are present in the serum of “responder” subjects at lower levels than in the serum of “non-responder” subjects. Thus, by assessing the activity, expression, or amount of one or more of the biomarkers described herein prior to treatment with an agent capable of inhibiting or reversing EMT or a chemotherapeutic agent it is possible to determine the likelihood of that subject being susceptible or responsive to treatment with said agent.

Accordingly, in a first aspect of this facet, the disclosure provides a method of predicting a cancer-related outcome in a subject having, suspected of having, or diagnosed with acute myelocytic leukemia (AML) or myelodysplastic syndrome (MDS), the method comprising assessing the activity, expression, or amount of one or more of the biomarkers described herein in the subject, or in a sample derived from the subject.

In some embodiments the one or more biomarker is selected from the group consisting of: Axl, Fibroblast Growth Factor 21 (FGF-21), Matrix Metalloproteinase-10 (MMP-10), Transferrin receptor protein 1 (TFR1), Tamm-Horsfall Urinary Glycoprotein (THP), Hemopexin, Haptoglobin, and/or Immunoglobulin M (IgM).

A “cancer-related outcome” as described herein is a clinical prediction or prognosis associated with cancer. In some embodiments the cancer-related outcome is susceptibility to treatment with an agent capable of inhibiting or reversing EMT or a chemotherapeutic agent.

In some embodiments the method comprises assessing the activity, expression, or amount of one or more of the biomarkers described herein in the subject, or in a sample derived from the subject, to obtain a sample profile of the one or more biomarker; and, making a prediction based on the sample profile of the one or more biomarker.

A “sample profile” as used herein is a profile of numerical values or numerical ranges corresponding to the activity, expression, or amount of respective biomarkers determined in the subject or sample derived from the subject.

In some embodiments of the method the prediction is made by comparing the sample profile to a control profile. A “control profile” as used herein is a profile of numerical values or numerical ranges corresponding to the activity, expression, or amount of the same biomarkers as for the sample profile, determined in a control subject or population of control subjects. Suitable control profiles are described in more detail below.

Prior to treatment of a subject, certain of the biomarkers described herein have been found to exhibit up-regulated expression in “responder” subjects—that is, certain of the biomarkers described herein have been found to be present in the serum of “responder” subjects at higher levels than in the serum of “non-responder” subjects. Similarly, prior to treatment of a subject, certain of the biomarkers described herein have been found to exhibit down-regulated expression in “responder” subjects—that is, certain of the biomarkers described herein have been found to be present in the serum of “responder” subjects at lower levels than in the serum of “non-responder” subjects.

This difference in expression level between “responder” and “non-responder” subjects can also be expressed in the opposite way. That is to say, prior to treatment of a subject, certain of the biomarkers described herein have been found to exhibit down-regulated expression in “non-responder” subjects—that is, certain of the biomarkers described herein have been found to be present in the serum of “non-responder” subjects at lower levels than in the serum of “responder” subjects. Similarly, prior to treatment of a subject, certain of the biomarkers described herein have been found to exhibit up-regulated expression in “non-responder” subjects—that is, certain of the biomarkers described herein have been found to be present in the serum of “non-responder” subjects at higher levels than in the serum of “responder” subjects.

The discovery of these differential patterns of expression mean that, by assessing the activity, expression, or amount of one or more of the biomarkers described herein prior to treatment of a subject with an agent, it is possible to determine the likelihood of that subject being susceptible or responsive to treatment with said agent. Accordingly, in particularly preferred embodiments the sample profile is obtained before the subject is contacted with or administered the agent capable of inhibiting or reversing EMT or a chemotherapeutic agent.

A subject who is “susceptible to treatment” with an agent capable of inhibiting or reversing EMT or a chemotherapeutic agent is one who is likely to, or who does, respond to treatment (or benefit from treatment) with an agent capable of inhibiting or reversing EMT or a chemotherapeutic agent. That is, a subject who is “susceptible to treatment” with an agent is a “responder” subject for that agent, as defined above. A prediction that is “indicative of susceptibility” is one which suggests or points towards the likelihood that a subject will respond to or benefit from treatment.

In some embodiments the predictive methods of the disclosure comprise assessing the activity, expression, or amount of one or more of the biomarkers described herein in the subject, or in a sample derived from the subject, to obtain a sample profile of the one or more biomarker; and, making a prediction based on the sample profile of the one or more biomarker. In some embodiments of the method the prediction is made by comparing the sample profile to a control profile, wherein the control profile is a profile of numerical values or numerical ranges corresponding to the activity, expression, or amount of the same biomarkers as for the sample profile, determined in a control subject or population of control subjects.

In some embodiments the control profile may be obtained from a population of control subjects having AML or MDS. In some embodiments the control profile may be obtained from a population of control subjects not having AML or MDS. In other embodiments the control profile may be obtained from a control subject or population of control subjects having AML or MDS and previously found to lack susceptibility to treatment with an agent capable of inhibiting or reversing EMT or a chemotherapeutic agent (that is, non-responder subjects).

In some embodiments the control profile may a predetermined profile of biomarker expression, activity, or amount values, for example a profile of “mean” values, “threshold” values, or “standard ranges” of values. This predetermined profile of biomarker expression, activity, or amount values can be obtained, for example, from a control subject or population of control subjects having AML or MDS, a control subject or population of control subjects not having AML or MDS, or a control subject or population of control subjects having AML or MDS and previously found to lack susceptibility to treatment with an agent capable of inhibiting or reversing EMT or a chemotherapeutic agent (that is, non-responder subjects).

In some other embodiments the control profile may be obtained from a control subject or population of control subjects having AML or MDS and previously found to be susceptible to treatment with an agent capable of inhibiting or reversing EMT or a chemotherapeutic agent (that is, responder subjects). In some embodiments the control profile may a predetermined profile of biomarker expression, activity, or amount values, for example a profile of “threshold”, values or “standard ranges” of values. This predetermined profile of biomarker expression, activity, or amount values can be obtained, for example, from a control subject or population of control subjects having AML or MDS and previously found to be susceptible to treatment with an agent capable of inhibiting or reversing EMT or a chemotherapeutic agent (that is, responder subjects).

In some other embodiments the control profile may be obtained from a control sample having a known amount (for example a “threshold” amount) of the same biomarkers as will be determined in the sample profile. This threshold amount can be obtained, for example, from non-responder or responder subjects as outlined above.

The skilled person is readily able to determine suitable “mean”, “threshold”, or “standard range” values for a particular biomarker.

For example, a “mean” value may be determined by: assessing the activity, expression, or amount of a particular biomarker in a population of subjects having (or not having) AML or MDS; and, averaging the biomarker activity, expression, or amount determined to determine a “mean” value.

For example, a “mean” value may be determined by: assessing the activity, expression, or amount of a particular biomarker in a population of subjects; determining which subjects in the population are susceptible to treatment with an agent capable of inhibiting or reversing EMT or a chemotherapeutic agent; and, averaging the biomarker activity, expression, or amount determined in subjects found to be susceptible to treatment with an agent capable of inhibiting or reversing EMT or a chemotherapeutic agent (that is, responder subjects) to determine a “mean” value.

For example, a “threshold” value may be determined by: assessing the activity, expression, or amount of a particular biomarker in a population of subjects; determining which subjects in the population are susceptible to treatment with an agent capable of inhibiting or reversing EMT or a chemotherapeutic agent; and, selecting as appropriate the highest or lowest biomarker activity, expression, or amount determined in a subject found to be susceptible to treatment with an agent capable of inhibiting or reversing EMT or a chemotherapeutic agent (that is, a responder subject) as the “threshold value”.

For example, a “standard range” of values may be determined by: assessing the activity, expression, or amount of a particular biomarker in a population of subjects having (or not having) AML or MDS; and, determining a “standard range” of values based on this assessment.

For example, a “standard range” of values may be determined by: assessing the activity, expression, or amount of a particular biomarker in a population of subjects; determining which subjects in the population are susceptible to treatment with an agent capable of inhibiting or reversing EMT or a chemotherapeutic agent; and, determining a “standard range” of values for subjects found to be susceptible to treatment with an agent capable of inhibiting or reversing EMT or a chemotherapeutic agent (that is, a responder subject) based on this assessment.

Where the control profile is a predetermined profile of biomarker expression, activity, or amount values, this may be provided as a “look-up table” or data record. Predetermined control profiles of this type may be a profile of numerical values or numerical ranges corresponding to a measure of central tendency (such as an average, median or mean) amount of the respective biomarkers determined in a plurality of control subjects (for example non-responder or responder subjects as outlined above). The skilled person is readily able to determine both a measure of central tendency and a measure of variability within a population using standard protein quantification techniques and suitable statistical tools.

Accordingly, in embodiments in which the control profile is: obtained from a population of control subjects having AML or MDS; obtained from a population of control subjects not having AML or MDS; obtained from a control subject or population of control subjects having AML or MDS and previously found to lack susceptibility to treatment with an agent capable of inhibiting or reversing EMT or a chemotherapeutic agent (that is non-responder subjects); a predetermined profile of biomarker expression, activity, or amount values (for example a profile of “average, median, or mean” values or “standard ranges” of values) obtained from non-responder subjects; obtained from a control sample having a known “average, median, or mean” value of biomarkers indicative of non-responder subjects; a predetermined profile of biomarker expression, activity, or amount “threshold” values obtained from responder subjects; or, obtained from a control sample having a known “threshold” value of biomarkers indicative of responder subjects; then, a higher activity, expression, or amount of one or more of the biomarkers described herein in a sample profile as compared with the control profile may be indicative of susceptibility to treatment with an agent capable of inhibiting or reversing EMT or a chemotherapeutic agent. Similarly, a lower activity, expression, or amount of one or more of the biomarkers described herein in the sample profile as compared with a control profile may be indicative of susceptibility to treatment with an agent capable of inhibiting or reversing EMT or a chemotherapeutic agent.

In some such embodiments, the method may comprise assessing the activity, expression, or amount of one or more of biomarker selected from: THP, Hemopexin, Haptoglobin, and/or IgM; wherein a higher activity, expression, or amount of one or more of said biomarkers in the sample profile as compared with the control profile is indicative of susceptibility to treatment with an agent capable of inhibiting or reversing EMT or a chemotherapeutic agent.

In some such embodiments, the method may comprise assessing the activity, expression, or amount of one or more of biomarker selected from: Axl, FGF-21, MMP-10, and/or TFR1; wherein a lower activity, expression, or amount of one or more of said biomarkers in the sample profile as compared with the control profile is indicative of susceptibility to treatment with an agent capable of inhibiting or reversing EMT or a chemotherapeutic agent.

In some such embodiments: a lower activity, expression, or amount of Axl; and/or a lower activity, expression, or amount of FGF-21; and/or a lower activity, expression, or amount of MMP-10; and/or a lower activity, expression, or amount of TFR1; and/or a higher activity, expression, or amount of THP; and/or a higher activity, expression, or amount of Hemopexin; and/or a higher activity, expression, or amount of Haptoglobin; and/or a higher activity, expression, or amount of IgM; in the sample profile as compared with the control profile is indicative of susceptibility to treatment with an agent capable of inhibiting or reversing EMT or a chemotherapeutic agent.

In embodiments in which the control profile is: obtained from a control subject or population of control subjects having AML or MDS and previously found to be susceptible to treatment with an agent capable of inhibiting or reversing EMT or a chemotherapeutic agent (that is, responder subjects); then, if the expression, activity, or amount values in the sample profile are above a “threshold” value typical of a responder subject (that is, a subject having AML or MDS and previously found to be susceptible to treatment with an agent capable of inhibiting or reversing EMT or a chemotherapeutic agent) this is indicative of susceptibility to treatment with an agent capable of inhibiting or reversing EMT or a chemotherapeutic agent. Similarly, if the expression, activity, or amount values in the sample profile are below the “threshold” value typical of a responder subject (that is, a subject having AML or MDS and previously found to be susceptible to treatment with an agent capable of inhibiting or reversing EMT or a chemotherapeutic agent) this is indicative of a lack of susceptibility to treatment with an agent capable of inhibiting or reversing EMT or a chemotherapeutic agent.

In embodiments in which the control profile is: a predetermined profile of “standard ranges” of biomarker expression, activity, or amount values obtained from responder subjects; then, if the expression, activity, or amount values in the sample profile are within the “standard range” of values typical of a responder subject (that is, a subject having AML or MDS and previously found to be susceptible to treatment with an agent capable of inhibiting or reversing EMT or a chemotherapeutic agent) this is indicative of susceptibility to treatment with an agent capable of inhibiting or reversing EMT or a chemotherapeutic agent. Similarly, if the expression, activity, or amount values in the sample profile are outside the “standard range” of values typical of a responder subject (that is, a subject having AML or MDS and previously found to be susceptible to treatment with an agent capable of inhibiting or reversing EMT or a chemotherapeutic agent) this is indicative of a lack of susceptibility to treatment with an agent capable of inhibiting or reversing EMT or a chemotherapeutic agent.

In embodiments in which the control profile is: a predetermined profile of “standard ranges” of biomarker expression, activity, or amount values obtained from non-responder subjects; then, if the expression, activity, or amount values in the sample profile are within the “standard range” of values typical of a non-responder subject (that is, a subject having AML or MDS and previously found to lack susceptibility to treatment with an agent capable of inhibiting or reversing EMT or a chemotherapeutic agent) this is indicative of a lack of susceptibility to treatment with an agent capable of inhibiting or reversing EMT or a chemotherapeutic agent. Similarly, if the expression, activity, or amount values in the sample profile are outside the “standard range” of values typical of a non-responder subject (that is, a subject having AML or MDS and previously found to lack susceptibility to treatment with an agent capable of inhibiting or reversing EMT or a chemotherapeutic agent) this may be indicative of susceptibility to treatment with an agent capable of inhibiting or reversing EMT or a chemotherapeutic agent.

In embodiments in which the control profile is: obtained from a population of control subjects not having AML or MDS; then, if the expression, activity, or amount values in the sample profile are outside a “standard range” of values typical of subjects not having AML or MDS, this is indicative of susceptibility to treatment with an agent capable of inhibiting or reversing EMT or a chemotherapeutic agent. Similarly, if the expression, activity, or amount values in the sample profile are within a “standard range” of values typical of subjects not having AML or MDS, this is indicative of a lack of susceptibility to treatment with an agent capable of inhibiting or reversing EMT or a chemotherapeutic agent. In such cases, an expression, activity or amount value may be outside the “standard range” of values when it is either above the upper limit of normal or below the lower limit of normal for a given biomarker. The upper limit of normal and lower limit of normal are the limits of the normal distribution of a given biomarker in a population of control subjects not having AML or MDS.

The skilled person is readily able to determine whether the sample profile is indicative of susceptibility or lack of susceptibility to treatment with an agent capable of inhibiting or reversing EMT or a chemotherapeutic agent using suitable statistical tools and comparison with the appropriate control.

In some embodiments the sample profile may be compared with two or more control profiles as described above.

As used herein, the term “one or more” means 1, 2, 3, 4, 5, 6, 7, 8, 9, 10, 11, 12, 13, 14, 15, 16, 17, 18, 19, or 20 of the biomarkers described herein. In some embodiments the one or more biomarker is Axl. In some embodiments the one or more biomarker is FGF-21. In some embodiments the one or more biomarker is MMP-10. In some embodiments the one or more biomarker is TFR1. In some embodiments the one or more biomarker is THP. In some embodiments the one or more biomarker is Hemopexin. In some embodiments the one or more biomarker is Haptoglobin. In some embodiments the one or more biomarker is IgM.

In some embodiments the method may comprise assessing the activity, expression, or amount of at least 2, 3, 4, 5, 6, 7, 8, 9, 10, 11, 12, 13, 14, 15, 20 of said biomarkers in the subject, or in a sample derived from the subject. In some embodiments the method may comprise assessing the activity, expression, or amount of Axl and at least 1, 2, 3, 4, 5, 6, 7, 8, 9, 10, 11, 12, 13, 14, 15, 20 further biomarkers described herein in the subject, or in a sample derived from the subject.

Subject

A “subject” as referred to herein is any species of animal classified as a mammal, and includes but is not restricted to, domestic and farm animals, primates and humans. In some preferred embodiments the subject is a human of any sex or race. In some embodiments, the human is an adult human.

In some embodiments the predictive methods described herein are performed before the subject receives a treatment or course of treatment for AML or MDS. In other embodiments the predictive method is performed during or after a treatment or course of treatment for AML or MDS. In some embodiments the treatment or course of treatment for AML or MDS is an agent capable of inhibiting or reversing EMT. In other embodiments the treatment or course of treatment for AML or MDS is a chemotherapeutic treatment that is not an agent capable of inhibiting or reversing EMT.

In some embodiments of the predictive methods described herein the subject has previously been treated with an agent capable of inhibiting or reversing EMT or a chemotherapeutic agent. In some embodiments the subject has previously been treated with an agent selected from: a PHGDH inhibitor, a Slfn11 inhibitor, an Axl inhibitor, or an Akt3 inhibitor.

In some embodiments of the predictive methods described herein the subject has previously been treated with an agent that is different to the agent capable of inhibiting or reversing EMT or chemotherapeutic agent for which the subject's response is being predicted. That is, the subject has previously been treated with an agent that is not the agent which the predictive method is determining the subject's susceptibility to. Accordingly, in some embodiments, the subject has not previously been treated with an agent capable of inhibiting or reversing EMT or a chemotherapeutic agent. In some preferred embodiments, the subject has not previously been treated with an Axl inhibitor. In other preferred embodiments, the subject has not previously been treated with an Akt3 inhibitor.

Post-Treatment Changes in Biomarkers

In some embodiments of this aspect of the disclosure, the predictive method further comprises a subsequent step of assessing the activity, expression, or amount of one or more biomarker in the subject, or in a sample derived from the subject, to obtain a second sample profile of one or more of the biomarkers described herein after the subject has received a treatment or course of treatment an agent capable of inhibiting or reversing EMT or a chemotherapeutic agent.

That is, in some embodiments, the predictive method comprises:
(a) determining if a subject is susceptible to treatment with an agent capable of inhibiting or reversing EMT or a chemotherapeutic agent by assessing the activity, expression, or amount of one or more biomarker in the subject, as outlined above;
(b) administering to the subject a therapeutically effective amount of an agent capable of inhibiting or reversing EMT or a chemotherapeutic agent; and
(c) subsequently assessing the activity, expression, or amount of one or more biomarker in the subject, or in a sample derived from the subject.

In some embodiments, the predictive method comprises:
(a) determining if a subject is susceptible to treatment with an agent capable of inhibiting or reversing EMT or a chemotherapeutic agent by assessing the activity, expression, or amount of one or more biomarker in the subject, or in a sample derived from the subject, wherein the one or more biomarker is selected from the group consisting of: Axl, FGF-21, MMP-10, TFR1, THP, Hemopexin, Haptoglobin, and/or IgM;
(b) administering to the subject a therapeutically effective amount of an agent capable of inhibiting or reversing EMT or a chemotherapeutic agent; and
(c) subsequently assessing the activity, expression, or amount of one or more biomarker in the subject, or in a sample derived from the subject.

In some preferred embodiments the methods comprise subsequently assessing the activity, expression, or amount of Axl in the subject, or in a sample derived from the subject. In some embodiments an increase in the activity, expression, or amount of Axl in the second sample profile as compared with a control profile after contacting the subject with a cancer therapeutic agent is indicative of higher susceptibility to treatment with an agent capable of inhibiting or reversing EMT or a chemotherapeutic agent. In some preferred embodiments the control profile is obtained from the same subject prior to treatment with an agent capable of inhibiting or reversing EMT or a chemotherapeutic agent.

Post-Treatment Predictive Biomarkers and Methods

By analysing patient data obtained from clinical trial BGBC003 (NCT02488408), the authors have discovered that, following treatment of subjects having AML or MDS with an agent capable of inhibiting or reversing EMT or a chemotherapeutic agent, the levels of certain of the biomarkers described herein increases in the serum of “responder” subjects but not in the serum of “non-responder” subjects—see Example 1. Similarly, following treatment with an agent capable of inhibiting or reversing EMT or a chemotherapeutic agent, the levels of certain of the biomarkers described herein decreases in the serum of “responder” subjects but not in the serum of “non-responder” subjects. Thus, by assessing the activity, expression, or amount of one or more of the biomarkers described herein prior to and following treatment with an agent capable of inhibiting or reversing EMT or a chemotherapeutic agent it is possible to determine or confirm that the subject is susceptible or responsive to treatment with said agent.

Accordingly, in a second aspect of this facet the disclosure provides a method of predicting a cancer-related outcome in a subject having, suspected of having, or diagnosed with acute myelocytic leukemia (AML) or myelodysplastic syndrome (MDS), the method comprising assessing the activity, expression, or amount of one or more of the biomarkers described herein in a subject, or in a sample derived from the subject after the subject has been contacted with an agent capable of inhibiting or reversing EMT or a chemotherapeutic agent. In some embodiments the cancer-related outcome is susceptibility to treatment with an agent capable of inhibiting or reversing EMT or a chemotherapeutic agent.

In some embodiments the method comprises assessing the activity, expression, or amount of one or more of the biomarkers described herein in the subject, or in a sample derived from the subject, to obtain a sample profile of the one or more biomarker; and, making a prediction based on the sample profile of the one or more biomarker. In some embodiments of the method the prediction is made by comparing the sample profile to a control profile. In preferred embodiments the sample profile is obtained after contacting the subject with an agent capable of inhibiting or reversing EMT or a chemotherapeutic agent.

A “control profile” as used herein is a profile of numerical values or numerical ranges corresponding to the activity, expression, or amount of the same biomarkers as for the sample profile, determined in a control subject or population of control subjects.

In preferred embodiments of this aspect of the disclosure the control profile is obtained from the same subject prior to treatment with an agent capable of inhibiting or reversing EMT or a chemotherapeutic agent. In some embodiments the control profile is obtained from a population of control subjects having AML or MDS. In other embodiments, the control profile is a predetermined profile of biomarker expression, activity, or amount values, for example a profile of “threshold” values or “standard ranges” of values.

Accordingly, in embodiments in which the control profile is obtained from the same subject prior to treatment with an agent capable of inhibiting or reversing EMT or a chemotherapeutic agent, an increase in the activity, expression, or amount of one or more biomarker in the sample profile as compared with a control profile after contacting the subject with an agent capable of inhibiting or reversing EMT or a cancer therapeutic agent may be indicative of susceptibility to treatment with an agent capable of inhibiting or reversing EMT or a chemotherapeutic agent. In such embodiments, a decrease in the activity, expression, or amount of one or more biomarker in the sample profile as compared with a control profile after contacting the subject with a cancer therapeutic agent may be indicative of higher susceptibility to treatment with an agent capable of inhibiting or reversing EMT or a chemotherapeutic agent.

In some such embodiments, an increase in the activity, expression, or amount of one or more biomarker in the sample profile as compared with a control profile after contacting the subject with a cancer therapeutic agent may be indicative of lower susceptibility to treatment with an agent capable of inhibiting or reversing EMT or a chemotherapeutic agent. In some such embodiments, a decrease in the activity, expression, or amount of one or more biomarker in the sample profile as compared with a control profile after contacting the subject with a cancer therapeutic agent may be indicative of lower susceptibility to treatment with an agent capable of inhibiting or reversing EMT or a chemotherapeutic agent.

In some preferred embodiments the one or more biomarker includes at least Axl. In some embodiments the one or more biomarker is Axl. In some embodiments an increase in the activity, expression, or amount of Axl in the sample profile as compared with a control profile after contacting the subject with a cancer therapeutic agent is indicative of higher susceptibility to treatment with an agent capable of inhibiting or reversing EMT or a chemotherapeutic agent.

Subject

A “subject” as referred to herein is any species of animal classified as a mammal, and includes but is not restricted to, domestic and farm animals, primates and humans. In some preferred embodiments the subject is a human of any sex or race. In some embodiments, the human is an adult human.

In some embodiments of this aspect of the disclosure, the subject was selected for treatment with the agent capable of inhibiting or reversing EMT or a chemotherapeutic agent, using a predictive method according to the first aspect of this facet of the disclosure outlined above.

Agents Capable of Inhibiting or Reversing EMT

The present disclosure provides methods for predicting a cancer-related outcome in a subject having, suspected of having, or diagnosed with acute myelocytic leukemia (AML) or myelodysplastic syndrome (MDS). A “cancer-related outcome” is a clinical prediction or prognosis associated with cancer.

The agents capable of inhibiting or reversing EMT for use in this AML or MDS facet of the disclosure are defined as described above in the AML facet (see identically titled section above on page 14, line 19 to page 17, line 7).

Assessing Biomarker Activity, Expression, Amount

In some embodiments of the predictive methods described herein, assessing the activity, expression, or amount of one or more biomarker in a subject, or in a sample derived from a subject, comprises: contacting a sample, or an extract from a sample, with at least 1, 2, 3, 4, 5, 6, 7, 8, 9, 10 or more reagents for detecting the activity, expression, or amount of one or more biomarkers, each of which is selective for a particular biomarker; and, detecting and/or quantifying said reagents for detecting.

In some preferred embodiments, assessing the expression or amount of the one or more biomarker comprises determining the level of the one or more biomarker protein or mRNA in the subject or sample derived from the subject. Preferably the expression or amount of the one or more biomarker is assessed by determining the level of protein expression. In particularly preferred embodiments the expression or amount of the one or more biomarker is assessed by determining the level of protein expression in serum.

The term “expression” refers to the transcription of a gene's DNA template to produce the corresponding mRNA and translation of this mRNA to produce the corresponding gene product (i.e., a peptide, polypeptide, or protein) as well as the “expression” of a protein in one or more forms that may have been modified post translation.

In some embodiments determining the level of the one or more biomarker protein or mRNA comprises: contacting a sample, or an extract from a sample, with at least 1, 2, 3, 4, 5, 6, 7, 8, 9, 10 or more specific binding members, each of which selectively binds to a respective biomarker protein or mRNA; and, detecting and/or quantifying formation of complex formed by said specific binding member and said biomarker protein or mRNA. In some embodiments the specific binding member may comprise an antibody molecule or a binding fragment thereof.

A range of suitable techniques for determining protein and mRNA expression levels are well known in the art, for example microarray analysis, Western blotting, and PCR techniques such as QPCR. In some embodiments, determining the expression or amount of the one or more biomarker protein or mRNA may comprise use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.

In some embodiments, the expression or amount of the one or more biomarker is determined as an absolute amount of the respective biomarker protein or mRNA in the sample. In such cases, the respective biomarker protein or mRNA may be quantified in the sample and the amount expressed as a measure of the sample, for example the volume, mass, or total protein content of the sample.

In some embodiments, the expression or amount of the one or more biomarker is determined as a relative amount of the respective biomarker protein or mRNA in the sample, wherein the relative amount is determined relative to a reference protein or mRNA in the sample. Determining the relative amount of biomarker protein or mRNA in this way allows for normalisation to, e.g., account for differences in total protein concentration and to remove bias from sample to sample.

Preferably the reference protein or mRNA is one whose expression or amount does not vary significantly between subjects having cancer and subjects not having cancer, in particular AML or MDS. That is, the reference protein or mRNA is preferably one whose expression or amount is not altered by AML or MDS. Accordingly, the reference protein or mRNA may comprise one or more protein or mRNA which is not one of the biomarkers described herein. When expressing the expression or amount of the one or more biomarker as a relative amount, the absolute amount of the respective biomarker protein or mRNA in the sample may be divided by the absolute amount of the reference protein or mRNA in the sample.

Assessing Biomarker Expression at the RNA Level

Gene expression can be detected at the RNA level. RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland). Typical assay formats utilising ribonucleic acid hybridisation include nuclear run-on assays, RT-PCR, RNase protection assays (Melton et al., Nuc. Acids Res. 12:7035), Northern blotting and In Situ hybridization. Gene expression can also be detected by microarray analysis as described below.

Assessing Biomarker Expression at the Protein Level

Preferably biomarker level is assessed by measuring protein expression. Altered gene or protein expression may also be detected by measuring the polypeptides encoded by the gene. This may be achieved by using molecules which bind to the polypeptides encoded by the biomarker gene. Suitable molecules/agents which bind either directly or indirectly to the polypeptides in order to detect the presence of the protein include naturally occurring molecules such as peptides and proteins, for example antibodies, or they may be synthetic molecules.

Antibodies may be derived from commercial sources or through techniques which are familiar to those skilled in the art. In one embodiment, and where altered expression manifests itself through the expression of alteration of post translationally-modified forms of a protein biomarker, antibodies specific for those different forms may be used. For the purposes of this disclosure, the term “antibody”, unless specified to the contrary, includes whole antibodies, or fragments of whole antibodies which retain their binding activity for a target antigen. Such fragments include Fv, F(ab′) and F(ab′)2 fragments, as well as single chain antibodies (scFv). Furthermore, the antibodies and fragments thereof may be humanised antibodies, for example as described in EP239400A. For example: monoclonal and polyclonal antibodies, recombinant antibodies, proteolytic and recombinant fragments of antibodies (Fab, Fv, scFv, diabodies), single-domain antibodies (VHH, sdAb, nanobodies, IgNAR, VNAR), and proteins unrelated to antibodies, which have been engineered to have antibody-like specific binding. Antibodies may be bound to a solid support and/or packaged into kits in a suitable container along with suitable reagents, controls, instructions and the like.

Arrays

Array technology and the various techniques and applications associated with it are well known in the art. Array technology overcomes the disadvantages with traditional methods in molecular biology, which generally work on a “one gene in one experiment” basis, resulting in low throughput and the inability to appreciate the “whole picture” of gene function. In the context of the products and methods of the present disclosure, array technology can be used, for example, in the analysis of the expression of biomarker proteins or mRNA.

In general, any library or group of samples may be arranged in an orderly manner into an array, by spatially separating the members of the library or group. Examples of suitable libraries for arraying include nucleic acid libraries (including DNA, cDNA, oligonucleotide, etc. libraries), peptide, polypeptide and protein libraries, as well as libraries comprising any molecules, such as ligand libraries, among others. Accordingly, where reference is made to a “library” in this document, unless the context dictates otherwise, such reference should be taken to include reference to a library in the form of an array.

Proteins, polypeptides, etc., may also be immobilised in arrays. For example, antibodies have been used in microarray analysis of the proteome using protein chips (Borrebaeck C A, 2000, Immunol Today 21(8):379-82). Polypeptide arrays are reviewed in, for example, MacBeath and Schreiber, 2000, Science, 289(5485):1760-1763.

Sample

Suitable samples include, but are not limited to, tissue samples such as tissue biopsy, blood, urine, buccal scrapes etc., as well as serum, plasma, or tissue culture supernatant samples. In preferred embodiments the expression or amount of the one or more biomarker is assessed by determining the level of protein expression in the sample. In some preferred embodiments the sample is a blood, serum, or plasma sample. In some particularly preferred embodiments the sample is a serum sample.

When assessing the expression or amount of the one or more biomarker proteins or mRNA in serum or plasma samples derived from a subject, samples are removed and subjected to analytical techniques such as flow cytometry, mass cytometry (CyTOF), ELISA, PET, and SELDI-TOF MS. In some embodiments, the method may comprise extracting RNA from said sample and detecting gene expression by QPCR. In some other embodiments, gene expression may be detected by detecting protein products by, for example, Western Blot.

In some embodiments, the level of expression of the one or more biomarker is assessed by determining the copy number of the gene encoding the one or more biomarker. Copy number (i.e. gene duplication events) may be determined using standard techniques known in the art, e.g. using a DNA chip as described in Jiang et al. (Jiang Q, Ho Y Y, Hao L, Nichols Berrios C, Chakravarti A. Copy number variants in candidate genes are genetic modifiers of Hirschsprung disease. PLoS One. 2011; 6(6)).

Is some preferred embodiments of the predictive methods described herein, the method is performed in vitro or ex vivo.

Methods of Selecting a Subject for Treatment

A third aspect of this facet of the present disclosure relates to methods of selecting subjects having, suspected of having, or diagnosed with acute myelocytic leukemia (AML) or myelodysplastic syndrome (MDS) for treatment with an agent capable of inhibiting or reversing EMT or a chemotherapeutic agent. In some embodiments the method comprises: identifying subjects susceptible to treatment with an agent capable of inhibiting or reversing EMT or a chemotherapeutic agent using a predictive method defined in accordance with the above described first and second aspects of this facet of the disclosure; and, selecting thus identified subjects for treatment.

In some embodiments this aspect relates to methods of selecting subjects having, suspected of having, or diagnosed with acute myelocytic leukemia (AML) or myelodysplastic syndrome (MDS) for continued treatment with an agent capable of inhibiting or reversing EMT or a chemotherapeutic agent. In some embodiments the method comprises: identifying subjects susceptible to treatment with an agent capable of inhibiting or reversing EMT or a chemotherapeutic agent using a predictive method defined in accordance with the above described second aspect of this facet of the disclosure; and, selecting thus identified subjects for treatment.

In some embodiments of this aspect of the disclosure, the treatment comprises administering to the subject a therapeutically effective amount of an agent capable of inhibiting or reversing EMT or a chemotherapeutic agent as described herein. Accordingly, in some embodiments the treatment comprises an Axl inhibitor, Akt3 inhibitor, Slfn11 inhibitor, or PHGDH inhibitor. In some preferred embodiments the treatment comprises an Axl inhibitor, for example, BGB324/R428/bemcentinib, cabozantinib, TP-0903, foretinib, merestinib, bosutinib, gilteritinib, crizotinib, amuvatinib, sunitinib, sitravatinib, LDC1267, or UNC2025. Other Axl inhibitors include the anti-Axl antibodies described in WO2015/193428, WO2015/193430, WO2016/097370, and WO2016/166296. In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In some other preferred embodiments, the treatment comprises an Akt3 inhibitor. In some embodiments the Akt3 inhibitor is an Akt3 inhibitor disclosed in WO2016/102672.

In some embodiments of this aspect of the disclosure, the treatment is administered as a single agent. In other embodiments, the treatment is administered in combination with a further cancer treatment. Suitable further cancer treatments are outlined in detail above. In some embodiments the further cancer treatment is a pyrimidine analogue, for example azacitidine, capecitabine, cytarabine, edatrexate, floxuridine, fluorouracil, gemcitabine, or troxacitabine, or a cytidine analogue, for example decitabine. In some particularly preferred embodiments the further cancer treatment is cytarabine. In other particularly preferred embodiments the further cancer treatment is decitabine.

Kits

A fourth aspect of this facet of the present disclosure relates to diagnostic kits and test devices. In some embodiments the disclosure provides a diagnostic kit comprising 1, 2, 3, 4, 5, 6, 7, 8, 9, 10 or more reagents for detecting the activity, expression, or amount of one or more of the biomarkers described herein, each of which is selective for a particular biomarker. In some embodiments each of the reagents for detecting is selective for a biomarker selected from the group consisting of: Axl, FGF-21, MMP-10, TFR1, THP, Hemopexin, Haptoglobin, and/or IgM.

In other embodiments the disclosure provides a test device comprising 1, 2, 3, 4, 5, 6, 7, 8, 9, 10 or more reagents for detecting the activity, expression, or amount of one or more of the biomarkers described herein, each of which is selective for a particular biomarker. In some embodiments each of the reagents for detecting is selective for a biomarker selected from the group consisting of: Axl, FGF-21, MMP-10, TFR1, THP, Hemopexin, Haptoglobin, and/or IgM.

In some embodiments the diagnostic kit or test device comprises 1, 2, 3, 4, 5, 6, 7, 8, 9, 10 or more specific binding members, each of which selectively binds to one of the biomarkers described herein; and, one or more reagents for detecting said one or more specific binding members, or one or more reagents for detecting and/or quantifying formation of a complex formed by said specific binding member and said biomarker. In some embodiments each specific binding member selectively binds to a biomarker selected from the group consisting of: Axl, FGF-21, MMP-10, TFR1, THP, Hemopexin, Haptoglobin, and/or IgM.

In some embodiments the diagnostic kit or test device comprises a specific binding member which selectively binds to Axl, and at least 1, 2, 3, 4, 5, 6, 7, 8, 9, 10, or more specific binding members, each of which selectively binds to a biomarker selected from the group consisting of: FGF-21, MMP-10, TFR1, THP, Hemopexin, Haptoglobin, and/or IgM.

In some embodiments of this aspect of the disclosure the diagnostic kit or test device comprises a plurality of said specific binding members provided in the form of an array on a substrate or conjugated to a plurality of particles, such as beads or microspheres. The particles may be encoded with a detectable label. In some embodiments the specific binding member comprises an antibody molecule or a binding fragment thereof.

In some embodiments of the diagnostic kit or test device detecting formation of a complex formed by a specific binding member and a biomarker is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.

In some embodiments of the diagnostic kit or test device, the kit or test device comprises no more than 15, 20, 25, 30, 40, or 50 reagents for detecting. In some embodiments of the diagnostic kit or test device, the kit or test device comprises no more than 15, 20, 25, 30, 40, or 50 specific binding members.

The present disclosure also provides use of such diagnostic kits and test devices in methods of predicting a cancer-related outcome in a subject. In preferred embodiments the cancer-related outcome is susceptibility to treatment with an agent capable of inhibiting or reversing EMT or a chemotherapeutic agent as described herein.

In some embodiments the agent capable of inhibiting or reversing EMT or chemotherapeutic agent is an Axl inhibitor, Akt3 inhibitor, Slfn11 inhibitor, or PHGDH inhibitor. In some preferred embodiments the agent capable of inhibiting or reversing EMT or chemotherapeutic agent is an Axl inhibitor, for example BGB324/R428/bemcentinib, cabozantinib, TP-0903, foretinib, merestinib, bosutinib, gilteritinib, crizotinib, amuvatinib, sunitinib, sitravatinib, LDC1267, or UNC2025. Other Axl inhibitors include the anti-Axl antibodies described in WO2015/193428, WO2015/193430, WO2016/097370, and WO2016/166296. In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In some embodiments of the diagnostic kit or test device for use in a predictive method, the subject has, is suspected of having, or has been diagnosed with AML or MDS.

In some embodiments the method may be as defined in accordance with the above described first and second aspects of this facet of the disclosure.

A fifth aspect of this facet of the present disclosure relates to the use of a reagent for detecting the activity, expression, or amount of a biomarker selected from the group consisting of: Axl, FGF-21, MMP-10, TFR1, THP, Hemopexin, Haptoglobin, and/or IgM, in a method of predicting a cancer-related outcome in a subject.

A sixth aspect of this facet of the disclosure relates to the use of 1, 2, 3, 4, 5, 6, 7, 8, 9, 10 or more reagents for detecting the activity, expression, or amount a biomarker, each of which is selective for a particular biomarker selected from the group consisting of: Axl, FGF-21, MMP-10, TFR1, THP, Hemopexin, Haptoglobin, and/or IgM, in a method of manufacture of a diagnostic kit or test device for use in a method of predicting a cancer-related outcome in a subject. In some embodiments of these uses the reagent for detecting comprises a specific binding member selective for a particular biomarker. The specific binding member may comprise an antibody molecule or a binding fragment thereof.

In some embodiments the method may be as defined in accordance with the above described first and second aspects of this facet of the disclosure.

Uses

A seventh aspect of this facet of the present disclosure relates to the use of one or more of: Axl, FGF-21, MMP-10, TFR1, THP, Hemopexin, Haptoglobin, and/or IgM, as a biomarker for determining if a subject is susceptible to treatment with an agent capable of inhibiting or reversing EMT or a chemotherapeutic agent.

In some embodiments the use relates to use of Axl, and at least 1, 2, 3, 4, 5, 6, 7, 8, 9, 10, or more of: FGF-21, MMP-10, TFR1, THP, Hemopexin, Haptoglobin, and/or IgM, as a biomarker for determining if a subject is susceptible to treatment with an agent capable of inhibiting or reversing EMT or a chemotherapeutic agent.

In some embodiments the use is as a biomarker in a method as defined in accordance with the above described first and second aspects of this facet of the disclosure.

Methods of Treating a Subject

A eighth aspect of this facet of the present disclosure relates to methods of treating a subject having, suspected of having, or diagnosed with acute myelocytic leukemia (AML) or myelodysplastic syndrome (MDS). In some embodiments the method comprises: identifying a subject for treatment using a predictive method defined in accordance with the above described first and second aspects of this facet of the disclosure; and, administering to the subject a therapeutically effective amount of an agent capable of inhibiting or reversing EMT or a chemotherapeutic agent.

Accordingly, in some embodiments, the method comprises: (a) obtaining a sample from the subject; (b) determining if the subject is susceptible to treatment with an agent capable of inhibiting or reversing EMT or a chemotherapeutic agent using a predictive method defined in accordance with the above described first and aspects of this facet of the disclosure; and, (c) administering to the subject a therapeutically effective amount of an agent capable of inhibiting or reversing EMT or a chemotherapeutic agent.

In some embodiments the method comprises administering a therapeutically effective amount of an agent capable of inhibiting or reversing EMT or chemotherapeutic agent to a subject identified as susceptible to said treatment using a predictive method defined in accordance with the above described first and second aspect of this facets of the disclosure.

In other embodiments the method comprises: (a) administering to a subject a therapeutically effective amount of an agent capable of inhibiting or reversing EMT or a chemotherapeutic agent; and, (b) determining if the subject is susceptible to said treatment using a predictive method defined in accordance with the above described second aspect of this facet of the disclosure. In some embodiments the method further comprises: (c) administering to the subject one or more further therapeutically effective amounts of an agent capable of inhibiting or reversing EMT or a chemotherapeutic agent provided that the subject is identified as susceptible to said treatment.

A ninth aspect of this facet of the present disclosure relates to an agent capable of inhibiting or reversing EMT or chemotherapeutic agent for use in a method of treating a subject having, suspected of having, or diagnosed with acute myelocytic leukemia (AML) or myelodysplastic syndrome (MDS). In some embodiments the method of treatment is a method as defined above.

A tenth aspect of this facet of the present disclosure relates to use of an agent capable of inhibiting or reversing EMT or chemotherapeutic agent for use in a method of manufacture of a medicament for use in a method of treating a subject having, suspected of having, or diagnosed with acute myelocytic leukemia (AML) or myelodysplastic syndrome (MDS). In some embodiments the method of treatment is a method as defined above.

In some embodiments of these methods, agents for use, or uses, the agent capable of inhibiting or reversing EMT or chemotherapeutic agent comprises an Axl inhibitor, Akt3 inhibitor, Slfn11 inhibitor, or PHGDH inhibitor. In some preferred embodiments the agent capable of inhibiting or reversing EMT or chemotherapeutic agent is an Axl inhibitor, for example BGB324/R428/bemcentinib, cabozantinib, TP-0903, foretinib, merestinib, bosutinib, gilteritinib, crizotinib, amuvatinib, sunitinib, sitravatinib, LDC1267, or UNC2025. Other Axl inhibitors include the anti-Axl antibodies described in WO2015/193428, WO2015/193430, WO2016/097370, and WO2016/166296. In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In some other preferred embodiments, the treatment comprises an Akt3 inhibitor. In some embodiments the Akt3 inhibitor is an Akt3 inhibitor disclosed in WO2016/102672.

In some embodiments of these methods, agents for use, or uses, the treatment is administered as a single agent. In other embodiments, the treatment is administered in combination with a further cancer treatment. Suitable further cancer treatments are outlined in detail above. In some embodiments the further cancer treatment is a pyrimidine analogue, for example azacitidine, capecitabine, cytarabine, edatrexate, floxuridine, fluorouracil, gemcitabine, or troxacitabine, or a cytidine analogue, for example decitabine. In some particularly preferred embodiments the further cancer treatment is cytarabine. In other particularly preferred embodiments the further cancer treatment is decitabine.

Statements of Disclosure

The following numbered statements relate to aspects of this facet of the present disclosure, and form part of the description:
101. A method of predicting a cancer-related outcome in a subject, the method comprising assessing the activity, expression, or amount of one or more biomarker in the subject, or in a sample derived from the subject;
   wherein the one or more biomarker is selected from the group consisting of: Axl, FGF-21, MMP-10, TFR1, THP, Hemopexin, Haptoglobin, and/or IgM; and
   wherein the subject has, is suspected of having, or has been diagnosed with acute myelocytic leukemia (AML) or myelodysplastic syndrome (MDS).
102. A method according to statement 101, wherein the method comprises:
   assessing the activity, expression, or amount of one or more biomarker in the subject, or in a sample derived from the subject, to obtain a sample profile of the one or more biomarker; and
   making a prediction based on the sample profile of the one or more biomarker.
103. A method according to statement 102, wherein the prediction is made by comparing the sample profile to a control profile.
104. A method according to statement 103, wherein the sample profile is obtained before the subject is contacted with or administered an agent capable of inhibiting or reversing EMT or a chemotherapeutic agent.
105. A method according to statement 104, wherein the control profile is:
   (i) obtained from a population of control subjects having AML or MDS; or
   (ii) obtained from a control subject or population of control subjects having AML or MDS and previously found to lack susceptibility to treatment with an agent capable of inhibiting or reversing EMT or a chemotherapeutic agent (that is, non-responder subjects);
   (iii) a predetermined profile of biomarker expression, activity, or amount values, for example a profile of “average, median, or mean” values or “standard ranges” of values, obtained from a control subject or population of control subjects having AML or MDS and previously found to lack susceptibility to treatment with an agent capable of inhibiting or reversing EMT or a chemotherapeutic agent (that is, non-responder subjects);
   (iv) obtained from a control sample having a known “average, median, or mean” value of biomarkers indicative of a control subject or population of control subjects having AML or MDS and previously found to lack susceptibility to treatment with an agent capable of inhibiting or reversing EMT or a chemotherapeutic agent (that is, non-responder subjects);
   (v) a predetermined profile of biomarker expression, activity, or amount “threshold” values obtained from a control subject or population of control subjects having AML or MDS and previously found to be susceptible to treatment with an agent capable of inhibiting or reversing EMT or a chemotherapeutic agent (that is, responder subjects); or
   (vi) obtained from a control sample having a known “threshold” value of biomarkers indicative of a control subject or population of control subjects having AML or MDS and previously found to be susceptible to treatment with an agent capable of inhibiting or reversing EMT or a chemotherapeutic agent (that is, responder subjects).
106. A method according to statement 105, wherein a higher activity, expression, or amount of one or more biomarker in the sample profile as compared with the control profile is indicative of susceptibility to treatment with an agent capable of inhibiting or reversing EMT or a chemotherapeutic agent.
107. A method according to statement 105, wherein a lower activity, expression, or amount of one or more biomarker in the sample profile as compared with the control profile is indicative of susceptibility to treatment with an agent capable of inhibiting or reversing EMT or a chemotherapeutic agent.
108. A method according to any one of statements 105-107, wherein the one or more biomarker includes at least one of: THP, Hemopexin, Haptoglobin, and/or IgM,
   and wherein a higher activity, expression, or amount of one or more of said biomarkers in the sample profile as compared with the control profile is indicative of susceptibility to treatment with an agent capable of inhibiting or reversing EMT or a chemotherapeutic agent.
109. A method according to any one of statements 105-108, wherein the one or more biomarker includes at least one of: Axl, FGF-21, MMP-10, and/or TFR1,
   and wherein a lower activity, expression, or amount of one or more of said biomarkers in the sample profile as compared with the control profile is indicative of susceptibility to treatment with an agent capable of inhibiting or reversing EMT or a chemotherapeutic agent.
110. A method according to any one of statements 105-109, wherein:
   a lower activity, expression, or amount of Axl; and/or
   a lower activity, expression, or amount of FGF-21; and/or
   a lower activity, expression, or amount of MMP-10; and/or
   a lower activity, expression, or amount of TFR1; and/or
   a higher activity, expression, or amount of THP; and/or
   a higher activity, expression, or amount of Hemopexin; and/or
   a higher activity, expression, or amount of Haptoglobin; and/or
   a higher activity, expression, or amount of IgM;
   in the sample profile as compared with the control profile is indicative of susceptibility to treatment with an agent capable of inhibiting or reversing EMT or a chemotherapeutic agent.
111. A method according to any one of statements 101-110, wherein the one or more biomarker comprises Axl,
   optionally wherein the one or more biomarker comprises Axl and at least 1, 2, 3, 4, 5, 6, 7, 8, 9, 10, or more further biomarkers selected from: FGF-21, MMP-10, TFR1, THP, Hemopexin, Haptoglobin, and/or IgM.
112. A method according to any one of statements 101-111, wherein:
   said Axl biomarker comprises an amino acid sequence having at least 70%, 80%, 90%, 95%, 99% or 100% sequence identity with the full-length of the amino acid sequence disclosed at UniProt accession no: P30530-1 or P30530-2 (entry version 196) or a fragment thereof;
   said Fibroblast Growth Factor 21 (FGF-21) biomarker comprises an amino acid sequence having at least 70%, 80%, 90%, 95%, 99% or 100% sequence identity with the full-length of the amino acid sequence disclosed at UniProt accession no: Q9NSA1 (entry version 145) or a fragment thereof;
   said Matrix Metalloproteinase-10 (MMP-10) biomarker comprises an amino acid sequence having at least 70%, 80%, 90%, 95%, 99% or 100% sequence identity with the full-length of the amino acid sequence disclosed at UniProt accession no: P09238 (entry version 185) or a fragment thereof;
   said Transferrin receptor protein 1 (TFR1) biomarker comprises an amino acid sequence having at least 70%, 80%, 90%, 95%, 99% or 100% sequence identity with the full-length of the amino acid sequence disclosed at UniProt accession no: P02786 (entry version 222) or a fragment thereof;
   said Tamm-Horsfall Urinary Glycoprotein (THP) biomarker comprises an amino acid sequence having at least 70%, 80%, 90%, 95%, 99% or 100% sequence identity with the full-length of the amino acid sequence disclosed at UniProt accession no: P07911-1, P07911-2, P07911-3, P07911-4, or P07911-5 (entry version 182) or a fragment thereof;
   said Hemopexin biomarker comprises an amino acid sequence having at least 70%, 80%, 90%, 95%, 99% or 100% sequence identity with the full-length of the amino acid sequence disclosed at UniProt accession no: P02790 (entry version 179) or a fragment thereof;
   said Haptoglobin biomarker comprises an amino acid sequence having at least 70%, 80%, 90%, 95%, 99% or 100% sequence identity with the full-length of the amino acid sequence disclosed at UniProt accession no: P00738-1 or P00738-2 (entry version 200) or a fragment thereof; and/or
   said Immunoglobulin M (IgM) biomarker comprises an amino acid sequence having at least 70%, 80%, 90%, 95%, 99% or 100% sequence identity with the full-length of the amino acid sequence disclosed at UniProt accession no: P01871-1 or P01871-2 (entry version 180) or a fragment thereof;
   or a nucleic acid (either DNA or RNA) having at least 70%, 80%, 90%, 95%, 99% or 100% sequence identity with the full-length nucleic acid sequence encoding said amino acid sequences.
113. A method according to any one of statement 101-112, wherein the method comprises determining the amount of at least 2, 3, 4, 5, 6, 7, 8, 9, 10, 11, 12, 13, 14, 15, 20 of said biomarkers in the subject, or sample derived from the subject.
114. A method according to any one of statements 104-113, wherein assessing the activity, expression, or amount of one or more biomarker in the subject, or in a sample derived from the subject, comprises:
   contacting a sample, or an extract from the sample, with at least 1, 2, 3, 4, 5, 6, 7, 8, 9, 10 or more reagents for detecting the activity, expression, or amount of one or more biomarkers, each of which is selective for a particular biomarker; and
   detecting and/or quantifying said reagents for detecting.
115. A method according to statement 114, wherein assessing the expression or amount of one or more biomarker in the subject, or in a sample derived from the subject, comprises:
   contacting a sample, or an extract from the sample, with at least 1, 2, 3, 4, 5, 6, 7, 8, 9, 10 or more specific binding members, each of which selectively binds to a respective biomarker protein or mRNA; and
   detecting and/or quantifying formation of complex formed by said specific binding member and said biomarker protein or mRNA.
116. A method according to statement 115 wherein the specific binding member comprises an antibody molecule or binding fragment thereof.
117. A method according to statement 115 or 116, wherein detecting formation of a complex formed by the specific binding member and the biomarker protein or mRNA is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.
118. A method according to any one of statements 115-117 wherein the expression or amount of the one or more biomarker is determined as the absolute amount of the respective biomarker protein or mRNA in the sample.
119. A method according to any one of statements 115-117 wherein the expression or amount of the one or more biomarker is determined as a relative amount of the respective biomarker protein or mRNA in the sample,
   optionally wherein the relative amount is determined relative to a reference protein or mRNA in the sample, the expression of which is not altered by AML or MDS.
120. A method according to any one of statements 101-119, wherein the cancer-related outcome is susceptibility to treatment with an agent capable of inhibiting or reversing EMT or a chemotherapeutic agent.
121. A method according to statement 120, wherein the agent capable of inhibiting or reversing EMT or chemotherapeutic agent is an Axl inhibitor, Akt3 inhibitor, Slfn11 inhibitor, or PHGDH inhibitor.
122. A method according to statement 121, wherein the agent capable of inhibiting or reversing EMT or chemotherapeutic agent is an Axl inhibitor.
123. A method according to statement 122, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
124. A method according to statement 123, wherein the Axl inhibitor is BGB324/R428/bemcentinib.
125. A method according to statement 123, wherein the Axl inhibitor is an anti-Axl antibody described in WO2015/193428, WO2015/193430, WO2016/097370, or WO2016/166296.
126. A method according to statement 121, wherein the agent capable of inhibiting or reversing EMT or chemotherapeutic agent is an Akt3 inhibitor.
127. A method according to statement 126, wherein the Akt3 inhibitor is an Akt3 inhibitor disclosed in WO2016/102672.
128. A method according to any one of statements 120-127, wherein the agent capable of inhibiting or reversing EMT is administered as a single agent.
129. A method according to any one of statements 120-127, wherein the agent capable of inhibiting or reversing EMT is administered in combination with a further cancer treatment.
130. A method according to statement 129, wherein the further cancer treatment is selected from:
   (i) alkylating agents, including alkyl sulfonates such as busulfan;
   (ii) nitrogen mustards such as chlorambucil, cyclophosphamide, estramustine, ifosfamide, mechlorethamine, melphalan, and uramustine;
   (iii) ethyleneimine derivatives such as thiotepa;
   (iv) nitrosoureas such as carmustine, lomustine, and streptozocin;
   (v) triazenes such as dacarbazine, procarbazine, and temozolamide;
   (vi) platinum compounds such as cisplatin, carboplatin, oxaliplatin, satraplatin, and picoplatin onnaplatin, tetraplatin, sprioplatin, iproplatin, chloro(diethylenediamino)-platinum (II) chloride, dichloro(ethylenediamino)-platinum (II), diamino(2-ethylmalonato)platinum (II), (1,2-diaminocyclohexane)malonatoplatinum (II), (4-carboxyphthalo)-(1,2-diaminocyclohexane)platinum (II), (1,2-diaminocyclohexane)-(isocitrato)platinum (II), and (1,2-diaminocyclohexane)-cis-(pyruvato)platinum (II);
   (vii) antimetabolites, including antifolates such as methotrexate, permetrexed, raltitrexed, and trimetrexate;
   (viii) pyrimidine analogues such as azacitidine, capecitabine, cytarabine, edatrexate, floxuridine, fluorouracil, gemcitabine, and troxacitabine;
   (ix) purine analogues such as cladribine, chlorodeoxyadenosine, clofarabine, fludarabine, mercaptopurine, pentostatin, and thioguanine;
   (x) natural products, including antitumor antibiotics such as bleomycin, dactinomycin, mithramycin, mitomycin, mitoxantrone, porfiromycin;
   (xi) anthracyclines such as daunorubicin, doxorubicin, epirubicin, idarubicin, and valrubicin;
   (xii) mitotic inhibitors such as the vinca alkaloids vinblastine, vinvesir, vincristine, vindesine, and vinorelbine;
   (xiii) enzymes such as L-asparaginase and PEG-L-asparaginase;
   (xiv) microtubule polymer stabilizers such as the taxanes paclitaxel and docetaxel;
   (xv) topoisomerase I inhibitors such as the camptothecins irinotecan and topotecan;
   (xvi) topoisomerase II inhibitors such as podophyllotoxin, amsacrine, etoposide, teniposide, losoxantrone and actinomycin;
   (xvii) hormones and hormone antagonists, including
      androgens such as fluoxymesterone and testolactone;
      antiandrogens such as bicalutamide, cyproterone, flutamide, and nilutamide;
      corticosteroids such as dexamethasone and prednisone;
      aromatase inhibitors such as aminoglutethimide, anastrozole, exemestane, formestane, and letrozole;
      estrogens such as diethylstilbestrol;
      antiestrogens such as fulvestrant, raloxifene, tamoxifen, and toremifine;
      luteinising hormone-releasing hormone (LHRH) agonists and antagonists such as abarelix, buserelin, goserelin, leuprolide, histrelin, desorelin, nafarelin acetate and triptorelin;
      progestins such as medroxyprogesterone acetate and megestrol acetate; and
      thyroid hormones such as levothyroxine and liothyronine;
   (xviii) PKB pathway inhibitors, including perifosine, enzastaurin hydrochloride, and triciribine;
   (xix) P13K inhibitors such as semaphore and SF1126, and MTOR inhibitors such as rapamycin and analogues;
   (xx) CDK inhibitors, including seliciclib, alvocidib, and 7-hydroxystaurosporine;
   (xxi) COX-2 inhibitors, including celecoxib;
   (xxii) HDAC inhibitiors, including trichostatin A, suberoylanilide hydroxamic acid, and chlamydocin;
   (xxiii) DNA methylase inhibitors, including temozolomide, and miscellaneous agents, including altretamine, arsenic trioxide, thalidomide, lenalidomide, gallium nitrate, levamisole, mitotane, hydroxyurea, octreotide, procarbazine, suramin, photodynamic compounds such as methoxsalen and sodium porfimer;
   (xxiv) proteasome inhibitors such as bortezomib;
   (xxv) molecular targeted therapy agents including: functional therapeutic agents, including gene therapy agents and antisense therapy agents;
   (xxvi) tyrosine kinase inhibitors such as erlotinib hydrochloride, gefitinib, imatinib mesylate, and semaxanib;
   (xxvii) Raf inhibitors such as sorafenib, and gene expression modulators such as the retinoids and rexinoids, for example adapalene, bexarotene, trans-retinoic acid, 9-cis-retinoic acid, and N-(4-hydroxyphenyl)retinamide;
   (xxviii) phenotype-directed therapy agents, including monoclonal antibodies such as pembrolizumab, alemtuzumab, bevacizumab, cetuximab, ibritumomab tiuxetan, rituximab, and trastuzumab, immunotoxins such as gemtuzumab ozogamicin, radioimmunoconjugates such as I-tositumobab;
   (xxix) cancer vaccines;
   (xxx) biologic therapy agents including: interferons such as interferon-[alpha]2a and interferon-[alpha]2b, and interleukins such as aldesleukin, denileukin diftitox, and oprelvekin; (xxxi) anticancer therapies involving the use of protective or adjunctive agents, including: cytoprotective agents such as amifostine, and dexrazoxane, phosphonates such as pamidronate and zoledronic acid, and stimulating factors such as epoetin, darbeopetin, filgrastim, PEG-filgrastim, and sargramostim;
   (xxxii) Axl inhibitors such as 1-(6,7-dihydro-5H-benzo[6,7]cyclohepta[1,2-c]pyridazin-3-yl)-N3-((7-(S)-pyrrolidin-1-yl)-6,7,8,9-tetrahydro-5H-benzo[7]annulene-2-yl)-1H-1,2,4-triazole-3,5-diamine; and/or
   (xxxiii) further combination chemotherapeutic regimens, such as combinations of carboplatin/paclitaxel, capecitabine/docetaxel, fluorauracil/levamisole, fluorauracil/leucovorin, methotrexate/leucovorin, and trastuzumab/paclitaxel, alone or in further combination with carboplatin.
131. A method according to statement 129, wherein the further cancer treatment is a pyrimidine analogue, for example azacitidine, capecitabine, cytarabine, edatrexate, floxuridine, fluorouracil, gemcitabine, or troxacitabine, or a cytidine analogue, for example decitabine.
132. A method according to statement 131, wherein the further cancer treatment is cytarabine.
133. A method according to statement 131, wherein the further cancer treatment is decitabine.
134. A method according to any one of statements 101-133, wherein the subject is mammalian.
135. A method according to statement 134, wherein the subject is human.
136. A method according to any one of statements 101-135, wherein the predictive method is performed before the subject receives a treatment or course of treatment for AML or MDS.
137. A method according to any one of statement 101-136, wherein the predictive method is performed during or after a treatment or course of treatment for AML or MDS.
138. A method according to any one of statements 101-135 or 137, wherein the subject has previously been treated with an agent capable of inhibiting or reversing EMT or a chemotherapeutic agent.
139. A method according to statement 138, wherein the subject has previously been treated with an agent selected from: a PHGDH inhibitor, a Slfn11 inhibitor, an Axl inhibitor, or an Akt3 inhibitor.
140. A method according to any one of statements 138-139, wherein the subject has previously been treated with an agent that is different to the agent capable of inhibiting or reversing EMT or chemotherapeutic agent for which the subject's susceptibility is being predicted.
141. A method according to any one of statements 101-136, wherein the subject has not previously been treated with an agent capable of inhibiting or reversing EMT or a chemotherapeutic agent.
142. A method according to statement 141, wherein the subject has not previously been treated with an Axl inhibitor.
143. A method according to statement 141, wherein the subject has not previously been treated with an Akt3 inhibitor.
144. A method according to any one of statements 101-143, wherein the sample is selected from: blood, serum, plasma, tissue biopsy, culture supernatant, or urine.
145. A method according to statement 144, wherein the sample is a blood, serum, or plasma sample.
146. A method according to statement 145, wherein the sample is a serum sample.
147. A method according to any one of statements 104-146, wherein the method further comprises a subsequent step of assessing the activity, expression, or amount of one or more biomarker in the subject, in which a sample profile is obtained after contacting the subject with an agent capable of inhibiting or reversing EMT or a chemotherapeutic agent.
148. A method according to statement 103, wherein the sample profile is obtained after contacting the subject with an agent capable of inhibiting or reversing EMT or a chemotherapeutic agent.
149. A method according to statement 147 or 148, wherein the control profile is obtained from the same subject prior to contacting said subject with the agent capable of inhibiting or reversing EMT or a chemotherapeutic agent.
150. A method according to statement 149, wherein an increase in the activity, expression, or amount of one or more biomarker in the sample profile as compared with the control profile is indicative of higher susceptibility to treatment with an agent capable of inhibiting or reversing EMT or a chemotherapeutic agent.
151. A method according to statement 149, wherein a decrease in the activity, expression, or amount of one or more biomarker in the sample profile as compared with the control profile is indicative of higher susceptibility to treatment with an agent capable of inhibiting or reversing EMT or a chemotherapeutic agent.
152. A method according to statement 149, wherein an increase in the activity, expression, or amount of one or more biomarker in the sample profile as compared with the control profile is indicative of lower susceptibility to treatment with an agent capable of inhibiting or reversing EMT or a chemotherapeutic agent.
153. A method according to statement 149, wherein a decrease in the activity, expression, or amount of one or more biomarker in the sample profile as compared with the control profile is indicative of lower susceptibility to treatment with an agent capable of inhibiting or reversing EMT or a chemotherapeutic agent.
154. A method according to statement 150, wherein the one or more biomarker includes at least Axl.
155. A method according to any one of statements 147-154, wherein assessing the activity, expression, or amount of one or more biomarker in the subject, or in a sample derived from the subject, comprises:
   contacting a sample, or an extract from the sample, with at least 1, 2, 3, 4, 5, 6, 7, 8, 9, 10 or more reagents for detecting the activity, expression, or amount of one or more biomarkers, each of which is selective for a particular biomarker; and
   detecting and/or quantifying said reagents for detecting.
156. A method according to statement 155, wherein assessing the expression or amount of one or more biomarker in the subject, or in a sample derived from the subject, comprises:
   contacting a sample, or an extract from the sample, with at least 1, 2, 3, 4, 5, 6, 7, 8, 9, 10 or more specific binding members, each of which selectively binds to a respective biomarker protein or mRNA; and
   detecting and/or quantifying formation of a complex formed by said specific binding member and said biomarker protein or mRNA.
157. A method according to statement 156, wherein the specific binding member comprises an antibody molecule or binding fragment thereof.
158. A method according to statement 156 or 157, wherein detecting formation of a complex formed by the specific binding member and the biomarker protein or mRNA is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.
159. A method according to any one of statements 156-158 wherein the expression or amount of the one or more biomarker is determined as the absolute amount of the respective biomarker protein or mRNA in the sample.
160. A method according to any one of statements 156-158 wherein the expression or amount of the one or more biomarker is determined as a relative amount of the respective biomarker protein or mRNA in the sample,
   optionally wherein the relative amount is determined relative to a reference protein or mRNA in the sample, the expression of which is not altered by AML or MDS.
161. A method according to any one of statements 148-160, wherein the agent capable of inhibiting or reversing EMT or chemotherapeutic agent is an Axl inhibitor, Akt3 inhibitor, Slfn11 inhibitor, or PHGDH inhibitor.
162. A method according to statement 161, wherein the agent capable of inhibiting or reversing EMT or chemotherapeutic agent is an Axl inhibitor.
163. A method according to statement 162, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
164. A method according to statement 163, wherein the Axl inhibitor is BGB324/R428/bemcentinib.
165. A method according to statement 163, wherein the Axl inhibitor is an anti-Axl antibody described in WO2015/193428, WO2015/193430, WO2016/097370, or WO2016/166296.
166. A method according to statement 161, wherein the agent capable of inhibiting or reversing EMT or chemotherapeutic agent is an Akt3 inhibitor.
167. A method according to statement 166, wherein the Akt3 inhibitor is an Akt3 inhibitor disclosed in WO2016/102672.
168. A method according to any one of statements 161-167, wherein the agent capable of inhibiting or reversing EMT is administered as a single agent.
169. A method according to any one of statements 161-167, wherein the agent capable of inhibiting or reversing EMT is administered in combination with a further cancer treatment.
170. A method according to statement 169, wherein the further cancer treatment is selected from the further cancer treatments listed in statement 130.
171. A method according to statement 169, wherein the further cancer treatment is a pyrimidine analogue, for example azacitidine, capecitabine, cytarabine, edatrexate, floxuridine, fluorouracil, gemcitabine, or troxacitabine, or a cytidine analogue, for example decitabine.
172. A method according to statement 171, wherein the further cancer treatment is cytarabine.
173. A method according to statement 171, wherein the further cancer treatment is decitabine.
174. A method according to any one of statements 148-173, wherein the subject is mammalian.
175. A method according to statement 174, wherein the subject is human.
176. A method according to any one of statements 148-175, wherein the subject was selected for treatment with the agent capable of inhibiting or reversing EMT or a chemotherapeutic agent, using a predictive method according to any one of statements 101-147.
177. A method according to any one of statements 148-176, wherein the sample is selected from: blood, serum, plasma, tissue biopsy, culture supernatant, or urine.
178. A method according to statement 177, wherein the sample is a blood, serum, or plasma sample.
179. A method according to statement 178, wherein the sample is a serum sample.
180. A method according to any one of statements 1-179, wherein the method is performed in vitro or ex vivo.
201. A method of selecting subjects having, suspected of having, or diagnosed with acute myelocytic leukemia (AML) or myelodysplastic syndrome (MDS) for treatment with an agent capable of inhibiting or reversing EMT or a chemotherapeutic agent, the method comprising:
   identifying subjects susceptible to treatment with an agent capable of inhibiting or reversing EMT or a chemotherapeutic agent using a method according to any one of statements 101-180; and
   selecting thus identified subjects for treatment.
202. A method of selecting subjects having, suspected of having, or diagnosed with acute myelocytic leukemia (AML) or myelodysplastic syndrome (MDS) for continued treatment with an agent capable of inhibiting or reversing EMT or a chemotherapeutic agent, the method comprising:
   identifying subjects susceptible to treatment with an agent capable of inhibiting or reversing EMT or a chemotherapeutic agent using a method according to any one of statements 148-180; and
   selecting thus identified subjects for treatment.
203. A method according to any one of statements 201-202, wherein treatment comprises administering to the subject a therapeutically effective amount of an agent capable of inhibiting or reversing EMT or a chemotherapeutic agent.
204. A method according to statement 203, wherein the treatment comprises an Axl inhibitor, Akt3 inhibitor, Slfn11 inhibitor, or PHGDH inhibitor.
205. A method according to statement 204, wherein the treatment comprises an Axl inhibitor.
206. A method according to statement 205, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
207. A method according to statement 206, wherein the Axl inhibitor is BGB324/R428/bemcentinib.
208. A method according to statement 206, wherein the Axl inhibitor is an anti-Axl antibody described in WO2015/193428, WO2015/193430, WO2016/097370, or WO2016/166296.
209. A method according to statement 204, wherein the treatment comprises an Akt3 inhibitor.
210. A method according to statement 209, wherein the Akt3 inhibitor is an Akt3 inhibitor disclosed in WO2016/102672.
211. A method according to any one of statements 203-210, wherein the treatment is administered as a single agent.
212. A method according to any one of statements 203-211, wherein the treatment is administered in combination with a further cancer treatment.
213. A method according to statement 212, wherein the further cancer treatment is selected from the further cancer treatments listed in statement 130.
214. A method according to statement 212, wherein the further cancer treatment is a pyrimidine analogue, for example azacitidine, capecitabine, cytarabine, edatrexate, floxuridine, fluorouracil, gemcitabine, or troxacitabine, or a cytidine analogue, for example decitabine.
215. A method according to statement 214, wherein the further cancer treatment is cytarabine.
216. A method according to statement 214, wherein the further cancer treatment is decitabine.
301. A diagnostic kit comprising 1, 2, 3, 4, 5, 6, 7, 8, 9, 10 or more reagents for detecting the activity, expression, or amount of one or more biomarkers, each of which is selective for a particular biomarker selected from the group consisting of: Axl, FGF-21, MMP-10, TFR1, THP, Hemopexin, Haptoglobin, and/or IgM.
302. A test device comprising 1, 2, 3, 4, 5, 6, 7, 8, 9, 10 or more reagents for detecting the activity, expression, or amount of one or more biomarkers, each of which is selective for a particular biomarker selected from the group consisting of: Axl, FGF-21, MMP-10, TFR1, THP, Hemopexin, Haptoglobin, and/or IgM.
303. A diagnostic kit or test device according to any one of statements 301 or 302, comprising:
   1, 2, 3, 4, 5, 6, 7, 8, 9, 10 or more specific binding members, each of which selectively binds to a biomarker selected from the group consisting of: Axl, FGF-21, MMP-10, TFR1, THP, Hemopexin, Haptoglobin, and/or IgM; and
   one or more reagents for detecting said one or more specific binding members or one or more reagents for detecting and/or quantifying formation of a complex formed by said specific binding member and said biomarker.
304. A diagnostic kit or test device according to statement 303, wherein the specific binding member comprises an antibody molecule or binding fragment thereof.
305. A diagnostic kit or test device according to statement 303 or 304, wherein detecting formation of a complex formed by the specific binding member and the biomarker is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.
306. A diagnostic kit or test device according to any one of statements 301-305, wherein the kit or test device comprises no more than 15, 20, 25, 30, 40, or 50 reagents for detecting.
307. A diagnostic kit or test device according to any one of statements 301-306 for use in a method of predicting a cancer-related outcome in a subject.
308. A diagnostic kit for use, or test device for use, according to statement 307, wherein the cancer-related outcome is susceptibility to treatment with an agent capable of inhibiting or reversing EMT or a chemotherapeutic agent.
309. A diagnostic kit for use, or test device for use, according to statement 308, wherein the agent capable of inhibiting or reversing EMT or chemotherapeutic agent is an Axl inhibitor, Akt3 inhibitor, Slfn11 inhibitor, or PHGDH inhibitor.
310. A diagnostic kit for use, or test device for use, according to statement 308, wherein the agent capable of inhibiting or reversing EMT or chemotherapeutic agent is an Axl inhibitor.
311. A diagnostic kit for use, or test device for use, according to statement 310, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
312. A diagnostic kit for use, or test device for use, according to statement 311, wherein the Axl inhibitor is BGB324/R428/bemcentinib.
313. A diagnostic kit for use, or test device for use, according to statement 311, wherein the Axl inhibitor is an anti-Axl antibody described in WO2015/193428, WO2015/193430, WO2016/097370, or WO2016/166296.
314. A diagnostic kit for use, or test device for use, according to statement 308, wherein the agent capable of inhibiting or reversing EMT or chemotherapeutic agent is an Akt3 inhibitor.
315. A diagnostic kit for use, or test device for use, according to any one of statements 307-310, wherein the subject has, is suspected of having, or has been diagnosed with acute myelocytic leukemia (AML) or myelodysplastic syndrome (MDS).
316. A diagnostic kit for use, or test device for use, according to any one of statements 307-315, wherein the method is a method according to any one of statements 101-180.
317. Use of a reagent for detecting the activity, expression, or amount of a biomarker selected from the group consisting of: Axl, FGF-21, MMP-10, TFR1, THP, Hemopexin, Haptoglobin, and/or IgM, in a method of predicting a cancer-related outcome in a subject.
318. Use of 1, 2, 3, 4, 5, 6, 7, 8, 9, 10 or more reagents for detecting the activity, expression, or amount a biomarker, each of which is selective for a particular biomarker selected from the group consisting of: Axl, FGF-21, MMP-10, TFR1, THP, Hemopexin, Haptoglobin, and/or IgM, in a method of manufacture of a diagnostic kit or test device for use in a method of predicting a cancer-related outcome in a subject.
319. A use according to statement 317 or 318, wherein the reagent for detecting comprises a specific binding member selective for a particular biomarker.
320. A use according to statement 317 or 318, wherein the method of predicting a cancer-related outcome in a subject is a method according to any one of statements 101-180.
401. Use of one or more of: Axl, FGF-21, MMP-10, TFR1, THP, Hemopexin, Haptoglobin, and/or IgM, as a biomarker for determining if a subject is susceptible to treatment with an agent capable of inhibiting or reversing EMT or a chemotherapeutic agent.
402. A use according to statement 401, wherein:
   (i) a higher activity, expression, or amount of one or more of said biomarkers in the subject, or sample derived from the subject, compared with a control profile for the respective biomarker is indicative of susceptibility to treatment with an agent capable of inhibiting or reversing EMT or a chemotherapeutic agent; or
   (ii) a lower activity, expression, or amount of one or more of said biomarkers in the subject, or sample derived from the subject, compared with a control profile for the respective biomarker is indicative of susceptibility to treatment with an agent capable of inhibiting or reversing EMT or a chemotherapeutic agent.
403. A use according to statement 401, wherein:
   (i) an increase in the activity, expression, or amount of one or more biomarker relative to a control profile for the respective biomarker after contacting the subject with a cancer therapeutic agent is indicative of increased susceptibility to treatment with an agent capable of inhibiting or reversing EMT or a chemotherapeutic agent; or
   (ii) a decrease in the activity, expression, or amount of one or more biomarker relative to a control profile for the respective biomarker after contacting the subject with a cancer therapeutic agent is indicative of increased susceptibility to treatment with an agent capable of inhibiting or reversing EMT or a chemotherapeutic agent.
404. A use according to any one of statements 401-403, wherein the use is as a biomarker in a method according to any one of statements 101-180
501. A method of treating a subject having, suspected of having, or diagnosed with acute myelocytic leukemia (AML) or myelodysplastic syndrome (MDS), the method comprising:
   identifying a subject susceptible to treatment using a method according to any one of statements 101-180; and
   administering to the subject a therapeutically effective amount of an agent capable of inhibiting or reversing EMT or a chemotherapeutic agent.
502. A method of treating a subject having, suspected of having, or diagnosed with acute myelocytic leukemia (AML) or myelodysplastic syndrome (MDS), the method comprising:
   (a) obtaining a sample from the subject;
   (b) determining if the subject is susceptible to treatment with an agent capable of inhibiting or reversing EMT or a chemotherapeutic agent using a method according to any one of statements 101-180;
   (c) administering to the subject a therapeutically effective amount of an agent capable of inhibiting or reversing EMT or a chemotherapeutic agent.
503. A method of treating a subject having, suspected of having, or diagnosed with acute myelocytic leukemia (AML) or myelodysplastic syndrome (MDS), the method comprising administering a therapeutically effective amount of an agent capable of inhibiting or reversing EMT or chemotherapeutic agent to a subject identified as susceptible to said treatment using a method according to any one of statements 101-180.
504. A method of treating a subject having, suspected of having, or diagnosed with acute myelocytic leukemia (AML) or myelodysplastic syndrome (MDS), the method comprising:
   administering to the subject a therapeutically effective amount of an agent capable of inhibiting or reversing EMT or a chemotherapeutic agent; and
   determining if the subject is susceptible to said treatment using a method according to any one of statements 148-180.
505. A method according to statement 504, wherein the method comprises administering to the subject one or more further therapeutically effective amounts of an agent capable of inhibiting or reversing EMT or a chemotherapeutic agent provided that the subject is identified as susceptible to said treatment.
506. An agent capable of inhibiting or reversing EMT or chemotherapeutic agent for use in a method of treating a subject having, suspected of having, or diagnosed with acute myelocytic leukemia (AML) or myelodysplastic syndrome (MDS),
   wherein the method is a method according to any one of statements 501-505.
507. Use of an agent capable of inhibiting or reversing EMT or chemotherapeutic agent for use in a method of manufacture of a medicament for use in a method of treating a subject having, suspected of having, or diagnosed with acute myelocytic leukemia (AML) or myelodysplastic syndrome (MDS),
   wherein the method of treatment is a method according to any one of statements 501-505.
508. A method, agent for use, or use according to any one of statements 501-507, wherein the agent capable of inhibiting or reversing EMT or chemotherapeutic agent comprises an Axl inhibitor, Akt3 inhibitor, Slfn11 inhibitor, or PHGDH inhibitor.
509. A method, agent for use, or use according to statement 508, wherein the agent capable of inhibiting or reversing EMT or chemotherapeutic agent comprises an Axl inhibitor.
510. A method, agent for use, or use according to statement 509, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
511. A method, agent for use, or use according to statement 510, wherein the Axl inhibitor is BGB324/R428/bemcentinib.
512. A method, agent for use, or use according to statement 510, wherein the Axl inhibitor is an anti-Axl antibody described in WO2015/193428, WO2015/193430, WO2016/097370, or WO2016/166296.
512. A method, agent for use, or use according to statement 508, wherein the agent capable of inhibiting or reversing EMT or chemotherapeutic agent comprises an Akt3 inhibitor.
513. A method, agent for use, or use according to statement 512, wherein the Akt3 inhibitor is an Akt3 inhibitor disclosed in WO2016/102672.
514. A method, agent for use, or use according to any one of statements 501-513, wherein the agent capable of inhibiting or reversing EMT or chemotherapeutic agent is administered as a single agent.
515. A method, agent for use, or use according to any one of statements 501-513, wherein the agent capable of inhibiting or reversing EMT or chemotherapeutic agent is administered in combination with a further cancer treatment.
516. A method, agent for use, or use according to statement 515, wherein the further cancer treatment is selected from the further cancer treatments listed in statement 130.
517. A method, agent for use, or use according to statement 515, wherein the further cancer treatment is a pyrimidine analogue, for example azacitidine, capecitabine, cytarabine, edatrexate, floxuridine, fluorouracil, gemcitabine, or troxacitabine, or a cytidine analogue, for example decitabine.
518. A method, agent for use, or use according to statement 517, wherein the further cancer treatment is cytarabine.
519. A method, agent for use, or use according to statement 517, wherein the further cancer treatment is decitabine.

Facet 3a: Acute Myelocytic Leukemia (AML) & Myelodysplastic Syndrome (MDS)

Biomarkers

The term “marker” or “biomarker” is used herein to refer to a gene or protein whose expression in a subject or sample derived from a subject is altered or modulated, for example, up or down regulated, in cancer. Where the biomarker is a protein, modulation or alteration of expression encompasses modulation through different post-translational modifications.

In accordance with any aspect of this facet of the present disclosure the biomarkers described herein may be defined as follows, wherein:
an Axl biomarker comprises an amino acid sequence having at least 70%, 80%, 90%, 95%, 99% or 100% sequence identity with the full-length of the amino acid sequence disclosed at UniProt accession no: P30530-1 or P30530-2 (entry version 196) or a fragment thereof;
a Tamm-Horsfall Urinary Glycoprotein (THP) biomarker comprises an amino acid sequence having at least 70%, 80%, 90%, 95%, 99% or 100% sequence identity with the full-length of the amino acid sequence disclosed at UniProt accession no: P07911-1, P07911-2, P07911-3, P07911-4, or P07911-5 (entry version 182) or a fragment thereof;
a Vitamin D-Binding Protein (VDBP) biomarker comprises an amino acid sequence having at least 70%, 80%, 90%, 95%, 99% or 100% sequence identity with the full-length of the amino acid sequence disclosed at UniProt accession no: P02774-1, P02774-2, or P02774-3 (entry version 201) or a fragment thereof;
a Epithelial cell adhesion molecule (EpCam) biomarker comprises an amino acid sequence having at least 70%, 80%, 90%, 95%, 99% or 100% sequence identity with the full-length of the amino acid sequence disclosed at UniProt accession no: P16422-1 (entry version 186) or a fragment thereof;
a Epidermal Growth Factor Receptor (EGFR) biomarker comprises an amino acid sequence having at least 70%, 80%, 90%, 95%, 99% or 100% sequence identity with the full-length of the amino acid sequence disclosed at UniProt accession no: Q504U8 (entry version 138) or a fragment thereof;
a Fetuin-A biomarker comprises an amino acid sequence having at least 70%, 80%, 90%, 95%, 99% or 100% sequence identity with the full-length of the amino acid sequence disclosed at UniProt accession no: P02765-1 (entry version 205) or a fragment thereof;
a Interleukin-6 receptor subunit beta (IL-6R beta) biomarker comprises an amino acid sequence having at least 70%, 80%, 90%, 95%, 99% or 100% sequence identity with the full-length of the amino acid sequence disclosed at UniProt accession no: P08887-1 or P08887-2 (entry version 210) or a fragment thereof;
a Complement Factor H-Related Protein 1 (CFHR1) biomarker comprises an amino acid sequence having at least 70%, 80%, 90%, 95%, 99% or 100% sequence identity with the full-length of the amino acid sequence disclosed at UniProt accession no: Q03591-1 (entry version 163) or a fragment thereof;
a Growth/differentiation factor 15 (GDF-15) biomarker comprises an amino acid sequence having at least 70%, 80%, 90%, 95%, 99% or 100% sequence identity with the full-length of the amino acid sequence disclosed at UniProt accession no: Q99988-1 (entry version 166) or a fragment thereof;
a Macrophage-Stimulating Protein (MSP) biomarker comprises an amino acid sequence having at least 70%, 80%, 90%, 95%, 99% or 100% sequence identity with the full-length of the amino acid sequence disclosed at UniProt accession no: P26927 (entry version 179) or a fragment thereof;
a Paraoxonase-1 (PON-1) biomarker comprises an amino acid sequence having at least 70%, 80%, 90%, 95%, 99% or 100% sequence identity with the full-length of the amino acid sequence disclosed at UniProt accession no: P27169-1 (entry version 202) or a fragment thereof;
a insulin (C-peptide) biomarker comprises an amino acid sequence having at least 70%, 80%, 90%, 95%, 99% or 100% sequence identity with the full-length of the amino acid sequence disclosed at UniProt accession no: P01308-1 (entry version 245), F8WCM5-1 (entry version 57) or a fragment thereof; and/or
an Apolipoprotein A-II (Apo A-II) biomarker comprises an amino acid sequence having at least 70%, 80%, 90%, 95%, 99% or 100% sequence identity with the full-length of the amino acid sequence disclosed at UniProt accession no: P02652-1 (entry version 217) or a fragment thereof.

The biomarkers described herein may also be defined as a nucleic acid (either DNA or RNA) having at least 70%, 80%, 90%, 95%, 99% or 100% sequence identity with the full-length nucleic acid sequence encoding any of the above amino acid sequences.

Identity may be as defined using sequence comparisons made using FASTA and FASTP (see Pearson & Lipman, 1988. Methods in Enzymology 183: 63-98). Parameters are preferably set, using the default matrix, as follows: Gapopen (penalty for the first residue in a gap): −12 for proteins/-16 for DNA; Gapext (penalty for additional residues in a gap): −2 for proteins/-4 for DNA; KTUP word length: 2 for proteins/6 for DNA.

Pre-Treatment Predictive Biomarkers and Methods

By analysing patient data obtained from clinical trial BGBC003 (NCT02488408), the authors have discovered that, prior to treatment of subjects having AML or MDS, certain of the biomarkers described herein are present in the serum of “responder” subjects at higher levels than in the serum of “non-responder” subjects—see Example 1. Similarly, prior to treatment of the subject certain of the biomarkers described herein are present in the serum of “responder” subjects at lower levels than in the serum of “non-responder” subjects. Thus, by assessing the activity, expression, or amount of one or more of the biomarkers described herein prior to treatment with an agent capable of inhibiting or reversing EMT or a chemotherapeutic agent it is possible to determine the likelihood of that subject being susceptible or responsive to treatment with said agent.

Accordingly, in a first aspect of this facet, the disclosure provides a method of predicting a cancer-related outcome in a subject having, suspected of having, or diagnosed with acute myelocytic leukemia (AML) or myelodysplastic syndrome (MDS), the method comprising assessing the activity, expression, or amount of one or more of the biomarkers described herein in the subject, or in a sample derived from the subject.

In some embodiments the one or more biomarker is selected from the group consisting of: VDBP, THP, EpCam, EGFR, Fetuin-A, IL-6R beta, Axl, CFHR1, GDF-15, MSP, PON-1, insulin (C-peptide), and/or Apo A-II.

A “cancer-related outcome” as described herein is a clinical prediction or prognosis associated with cancer. In some embodiments the cancer-related outcome is susceptibility to treatment with an agent capable of inhibiting or reversing EMT or a chemotherapeutic agent.

In some embodiments the method comprises assessing the activity, expression, or amount of one or more of the biomarkers described herein in the subject, or in a sample derived from the subject, to obtain a sample profile of the one or more biomarker; and, making a prediction based on the sample profile of the one or more biomarker.

A “sample profile” as used herein is a profile of numerical values or numerical ranges corresponding to the activity, expression, or amount of respective biomarkers determined in the subject or sample derived from the subject.

In some embodiments of the method the prediction is made by comparing the sample profile to a control profile. A “control profile” as used herein is a profile of numerical values or numerical ranges corresponding to the activity, expression, or amount of the same biomarkers as for the sample profile, determined in a control subject or population of control subjects. Suitable control profiles are described in more detail below.

Prior to treatment of a subject, certain of the biomarkers described herein have been found to exhibit up-regulated expression in “responder” subjects—that is, certain of the biomarkers described herein have been found to be present in the serum of “responder” subjects at higher levels than in the serum of “non-responder” subjects. Similarly, prior to treatment of a subject, certain of the biomarkers described herein have been found to exhibit down-regulated expression in “responder” subjects—that is, certain of the biomarkers described herein have been found to be present in the serum of “responder” subjects at lower levels than in the serum of “non-responder” subjects.

This difference in expression level between “responder” and “non-responder” subjects can also be expressed in the opposite way. That is to say, prior to treatment of a subject, certain of the biomarkers described herein have been found to exhibit down-regulated expression in “non-responder” subjects—that is, certain of the biomarkers described herein have been found to be present in the serum of “non-responder” subjects at lower levels than in the serum of “responder” subjects. Similarly, prior to treatment of a subject, certain of the biomarkers described herein have been found to exhibit up-regulated expression in “non-responder” subjects—that is, certain of the biomarkers described herein have been found to be present in the serum of “non-responder” subjects at higher levels than in the serum of “responder” subjects.

The discovery of these differential patterns of expression mean that, by assessing the activity, expression, or amount of one or more of the biomarkers described herein prior to treatment of a subject with an agent, it is possible to determine the likelihood of that subject being susceptible or responsive to treatment with said agent. Accordingly, in particularly preferred embodiments the sample profile is obtained before the subject is contacted with or administered the agent capable of inhibiting or reversing EMT or a chemotherapeutic agent.

A subject who is “susceptible to treatment” with an agent capable of inhibiting or reversing EMT or a chemotherapeutic agent is one who is likely to, or who does, respond to treatment (or benefit from treatment) with an agent capable of inhibiting or reversing EMT or a chemotherapeutic agent. That is, a subject who is “susceptible to treatment” with an agent is a “responder” subject for that agent, as defined above. A prediction that is “indicative of susceptibility” is one which suggests or points towards the likelihood that a subject will respond to or benefit from treatment.

In some embodiments the predictive methods of the disclosure comprise assessing the activity, expression, or amount of one or more of the biomarkers described herein in the subject, or in a sample derived from the subject, to obtain a sample profile of the one or more biomarker; and, making a prediction based on the sample profile of the one or more biomarker. In some embodiments of the method the prediction is made by comparing the sample profile to a control profile, wherein the control profile is a profile of numerical values or numerical ranges corresponding to the activity, expression, or amount of the same biomarkers as for the sample profile, determined in a control subject or population of control subjects.

In some embodiments the control profile may be obtained from a population of control subjects having AML or MDS. In some embodiments the control profile may be obtained from a population of control subjects not having AML or MDS. In other embodiments the control profile may be obtained from a control subject or population of control subjects having AML or MDS and previously found to lack susceptibility to treatment with an agent capable of inhibiting or reversing EMT or a chemotherapeutic agent (that is, non-responder subjects).

In some embodiments the control profile may a predetermined profile of biomarker expression, activity, or amount values, for example a profile of “mean” values, “threshold” values, or “standard ranges” of values. This predetermined profile of biomarker expression, activity, or amount values can be obtained, for example, from a control subject or population of control subjects having AML or MDS, a control subject or population of control subjects not having AML or MDS, or a control subject or population of control subjects having AML or MDS and previously found to lack susceptibility to treatment with an agent capable of inhibiting or reversing EMT or a chemotherapeutic agent (that is, non-responder subjects).

In some other embodiments the control profile may be obtained from a control subject or population of control subjects having AML or MDS and previously found to be susceptible to treatment with an agent capable of inhibiting or reversing EMT or a chemotherapeutic agent (that is, responder subjects). In some embodiments the control profile may a predetermined profile of biomarker expression, activity, or amount values, for example a profile of “threshold”, values or “standard ranges” of values. This predetermined profile of biomarker expression, activity, or amount values can be obtained, for example, from a control subject or population of control subjects having AML or MDS and previously found to be susceptible to treatment with an agent capable of inhibiting or reversing EMT or a chemotherapeutic agent (that is, responder subjects).

In some other embodiments the control profile may be obtained from a control sample having a known amount (for example a “threshold” amount) of the same biomarkers as will be determined in the sample profile. This threshold amount can be obtained, for example, from non-responder or responder subjects as outlined above.

The skilled person is readily able to determine suitable “mean”, “threshold”, or “standard range” values for a particular biomarker.

For example, a “mean” value may be determined by: assessing the activity, expression, or amount of a particular biomarker in a population of subjects having (or not having) AML or MDS; and, averaging the biomarker activity, expression, or amount determined to determine a “mean” value.

For example, a “mean” value may be determined by: assessing the activity, expression, or amount of a particular biomarker in a population of subjects; determining which subjects in the population are susceptible to treatment with an agent capable of inhibiting or reversing EMT or a chemotherapeutic agent; and, averaging the biomarker activity, expression, or amount determined in subjects found to be susceptible to treatment with an agent capable of inhibiting or reversing EMT or a chemotherapeutic agent (that is, responder subjects) to determine a “mean” value.

For example, a “threshold” value may be determined by: assessing the activity, expression, or amount of a particular biomarker in a population of subjects; determining which subjects in the population are susceptible to treatment with an agent capable of inhibiting or reversing EMT or a chemotherapeutic agent; and, selecting as appropriate the highest or lowest biomarker activity, expression, or amount determined in a subject found to be susceptible to treatment with an agent capable of inhibiting or reversing EMT or a chemotherapeutic agent (that is, a responder subject) as the “threshold value”.

For example, a “standard range” of values may be determined by: assessing the activity, expression, or amount of a particular biomarker in a population of subjects having (or not having) AML or MDS; and, determining a “standard range” of values based on this assessment.

For example, a “standard range” of values may be determined by: assessing the activity, expression, or amount of a particular biomarker in a population of subjects; determining which subjects in the population are susceptible to treatment with an agent capable of inhibiting or reversing EMT or a chemotherapeutic agent; and, determining a “standard range” of values for subjects found to be susceptible to treatment with an agent capable of inhibiting or reversing EMT or a chemotherapeutic agent (that is, a responder subject) based on this assessment.

Where the control profile is a predetermined profile of biomarker expression, activity, or amount values, this may be provided as a “look-up table” or data record. Predetermined control profiles of this type may be a profile of numerical values or numerical ranges corresponding to a measure of central tendency (such as an average, median or mean) amount of the respective biomarkers determined in a plurality of control subjects (for example non-responder or responder subjects as outlined above). The skilled person is readily able to determine both a measure of central tendency and a measure of variability within a population using standard protein quantification techniques and suitable statistical tools.

Accordingly, in embodiments in which the control profile is: obtained from a population of control subjects having AML or MDS; obtained from a population of control subjects not having AML or MDS; obtained from a control subject or population of control subjects having AML or MDS and previously found to lack susceptibility to treatment with an agent capable of inhibiting or reversing EMT or a chemotherapeutic agent (that is non-responder subjects); a predetermined profile of biomarker expression, activity, or amount values (for example a profile of “average, median, or mean” values or “standard ranges” of values) obtained from non-responder subjects; obtained from a control sample having a known “average, median, or mean” value of biomarkers indicative of non-responder subjects; a predetermined profile of biomarker expression, activity, or amount “threshold” values obtained from responder subjects; or, obtained from a control sample having a known “threshold” value of biomarkers indicative of responder subjects; then, a higher activity, expression, or amount of one or more of the biomarkers described herein in a sample profile as compared with the control profile may be indicative of susceptibility to treatment with an agent capable of inhibiting or reversing EMT or a chemotherapeutic agent. Similarly, a lower activity, expression, or amount of one or more of the biomarkers described herein in the sample profile as compared with a control profile may be indicative of susceptibility to treatment with an agent capable of inhibiting or reversing EMT or a chemotherapeutic agent.

In some such embodiments, the method may comprise assessing the activity, expression, or amount of one or more of biomarker selected from: VDBP, THP, EpCam, EGFR, Fetuin-A, IL-6R beta, MSP, PON-1, and/or Apo A-II.; wherein a higher activity, expression, or amount of one or more of said biomarkers in the sample profile as compared with the control profile is indicative of susceptibility to treatment with an agent capable of inhibiting or reversing EMT or a chemotherapeutic agent.

In some such embodiments, the method may comprise assessing the activity, expression, or amount of one or more of biomarker selected from: Axl, GDF-15, Insulin (c-peptide) and/or CFHR1; wherein a lower activity, expression, or amount of one or more of said biomarkers in the sample profile as compared with the control profile is indicative of susceptibility to treatment with an agent capable of inhibiting or reversing EMT or a chemotherapeutic agent.

In some such embodiments: a lower activity, expression, or amount of Axl; and/or a lower activity, expression, or amount of GDF-15; and/or a lower activity, expression, or amount of Insulin (c-peptide); and/or a lower activity, expression, or amount of CFHR1; and/or a higher activity, expression, or amount of VDBP; and/or a higher activity, expression, or amount of THP; and/or a higher activity, expression, or amount of EpCam; and/or a higher activity, expression, or amount of EGFR; and/or a higher activity, expression, or amount of Fetuin-A and/or a higher activity, expression, or amount of IL-6R beta; and/or a higher activity, expression, or amount of MSP; and/or a higher activity, expression, or amount of PON-1; and/or a higher activity, expression, or amount of Apo A-II; in the sample profile as compared with the control profile is indicative of susceptibility to treatment with an agent capable of inhibiting or reversing EMT or a chemotherapeutic agent.

In embodiments in which the control profile is: obtained from a control subject or population of control subjects having AML or MDS and previously found to be susceptible to treatment with an agent capable of inhibiting or reversing EMT or a chemotherapeutic agent (that is, responder subjects); then, if the expression, activity, or amount values in the sample profile are above a “threshold” value typical of a responder subject (that is, a subject having AML or MDS and previously found to be susceptible to treatment with an agent capable of inhibiting or reversing EMT or a chemotherapeutic agent) this is indicative of susceptibility to treatment with an agent capable of inhibiting or reversing EMT or a chemotherapeutic agent. Similarly, if the expression, activity, or amount values in the sample profile are below the “threshold” value typical of a responder subject (that is, a subject having AML or MDS and previously found to be susceptible to treatment with an agent capable of inhibiting or reversing EMT or a chemotherapeutic agent) this is indicative of a lack of susceptibility to treatment with an agent capable of inhibiting or reversing EMT or a chemotherapeutic agent.

In embodiments in which the control profile is: a predetermined profile of “standard ranges” of biomarker expression, activity, or amount values obtained from responder subjects; then, if the expression, activity, or amount values in the sample profile are within the “standard range” of values typical of a responder subject (that is, a subject having AML or MDS and previously found to be susceptible to treatment with an agent capable of inhibiting or reversing EMT or a chemotherapeutic agent) this is indicative of susceptibility to treatment with an agent capable of inhibiting or reversing EMT or a chemotherapeutic agent. Similarly, if the expression, activity, or amount values in the sample profile are outside the “standard range” of values typical of a responder subject (that is, a subject having AML or MDS and previously found to be susceptible to treatment with an agent capable of inhibiting or reversing EMT or a chemotherapeutic agent) this is indicative of a lack of susceptibility to treatment with an agent capable of inhibiting or reversing EMT or a chemotherapeutic agent.

In embodiments in which the control profile is: a predetermined profile of “standard ranges” of biomarker expression, activity, or amount values obtained from non-responder subjects; then, if the expression, activity, or amount values in the sample profile are within the “standard range” of values typical of a non-responder subject (that is, a subject having AML or MDS and previously found to lack susceptibility to treatment with an agent capable of inhibiting or reversing EMT or a chemotherapeutic agent) this is indicative of a lack of susceptibility to treatment with an agent capable of inhibiting or reversing EMT or a chemotherapeutic agent. Similarly, if the expression, activity, or amount values in the sample profile are outside the “standard range” of values typical of a non-responder subject (that is, a subject having AML or MDS and previously found to lack susceptibility to treatment with an agent capable of inhibiting or reversing EMT or a chemotherapeutic agent) this may be indicative of susceptibility to treatment with an agent capable of inhibiting or reversing EMT or a chemotherapeutic agent.

In embodiments in which the control profile is: obtained from a population of control subjects not having AML or MDS; then, if the expression, activity, or amount values in the sample profile are outside a “standard range” of values typical of subjects not having AML or MDS, this is indicative of susceptibility to treatment with an agent capable of inhibiting or reversing EMT or a chemotherapeutic agent. Similarly, if the expression, activity, or amount values in the sample profile are within a “standard range” of values typical of subjects not having AML or MDS, this is indicative of a lack of susceptibility to treatment with an agent capable of inhibiting or reversing EMT or a chemotherapeutic agent. In such cases, an expression, activity or amount value may be outside the “standard range” of values when it is either above the upper limit of normal or below the lower limit of normal for a given biomarker. The upper limit of normal and lower limit of normal are the limits of the normal distribution of a given biomarker in a population of control subjects not having AML or MDS.

The skilled person is readily able to determine whether the sample profile is indicative of susceptibility or lack of susceptibility to treatment with an agent capable of inhibiting or reversing EMT or a chemotherapeutic agent using suitable statistical tools and comparison with the appropriate control.

In some embodiments the sample profile may be compared with two or more control profiles as described above.

As used herein, the term “one or more” means 1, 2, 3, 4, 5, 6, 7, 8, 9, 10, 11, 12, 13, 14, 15, 16, 17, 18, 19, or 20 of the biomarkers described herein. In some embodiments the one or more biomarker is Axl. In some embodiments the one or more biomarker is VDBP. In some embodiments the one or more biomarker is THP. In some embodiments the one or more biomarker is EpCam. In some embodiments the one or more biomarker is EGFR. In some embodiments the one or more biomarker is Fetuin-A. In some embodiments the one or more biomarker is IL-6R beta.

In some embodiments the method may comprise assessing the activity, expression, or amount of at least 2, 3, 4, 5, 6, 7, 8, 9, 10, 11, 12, 13, 14, 15, 20 of said biomarkers in the subject, or in a sample derived from the subject. In some embodiments the method may comprise assessing the activity, expression, or amount of Axl and at least 1, 2, 3, 4, 5, 6, 7, 8, 9, 10, 11, 12, 13, 14, 15, 20 further biomarkers described herein in the subject, or in a sample derived from the subject.

Subject

A “subject” as referred to herein is any species of animal classified as a mammal, and includes but is not restricted to, domestic and farm animals, primates and humans. In some preferred embodiments the subject is a human of any sex or race. In some embodiments, the human is an adult human.

In some embodiments the predictive methods described herein are performed before the subject receives a treatment or course of treatment for AML or MDS. In other embodiments the predictive method is performed during or after a treatment or course of treatment for AML or MDS. In some embodiments the treatment or course of treatment for AML or MDS is an agent capable of inhibiting or reversing EMT. In other embodiments the treatment or course of treatment for AML or MDS is a chemotherapeutic treatment that is not an agent capable of inhibiting or reversing EMT.

In some embodiments of the predictive methods described herein the subject has previously been treated with an agent capable of inhibiting or reversing EMT or a chemotherapeutic agent. In some embodiments the subject has previously been treated with an agent selected from: a PHGDH inhibitor, a Slfn11 inhibitor, an Axl inhibitor, or an Akt3 inhibitor.

In some embodiments of the predictive methods described herein the subject has previously been treated with an agent that is different to the agent capable of inhibiting or reversing EMT or chemotherapeutic agent for which the subject's response is being predicted. That is, the subject has previously been treated with an agent that is not the agent which the predictive method is determining the subject's susceptibility to. Accordingly, in some embodiments, the subject has not previously been treated with an agent capable of inhibiting or reversing EMT or a chemotherapeutic agent. In some preferred embodiments, the subject has not previously been treated with an Axl inhibitor. In other preferred embodiments, the subject has not previously been treated with an Akt3 inhibitor.

Post-Treatment Changes in Biomarkers

In some embodiments of this aspect of the disclosure, the predictive method further comprises a subsequent step of assessing the activity, expression, or amount of one or more biomarker in the subject, or in a sample derived from the subject, to obtain a second sample profile of one or more of the biomarkers described herein after the subject has received a treatment or course of treatment an agent capable of inhibiting or reversing EMT or a chemotherapeutic agent.

That is, in some embodiments, the predictive method comprises:
(a) determining if a subject is susceptible to treatment with an agent capable of inhibiting or reversing EMT or a chemotherapeutic agent by assessing the activity, expression, or amount of one or more biomarker in the subject, as outlined above;
(b) administering to the subject a therapeutically effective amount of an agent capable of inhibiting or reversing EMT or a chemotherapeutic agent; and
(c) subsequently assessing the activity, expression, or amount of one or more biomarker in the subject, or in a sample derived from the subject.

In some embodiments, the predictive method comprises:
(a) determining if a subject is susceptible to treatment with an agent capable of inhibiting or reversing EMT or a chemotherapeutic agent by assessing the activity, expression, or amount of one or more biomarker in the subject, or in a sample derived from the subject, wherein the one or more biomarker is selected from the group consisting of: VDBP, THP, EpCam, EGFR, Fetuin-A, IL-6R beta, Axl, CFHR1, GDF-15, MSP, PON-1, insulin (C-peptide), and/or Apo A-II;
(b) administering to the subject a therapeutically effective amount of an agent capable of inhibiting or reversing EMT or a chemotherapeutic agent; and
(c) subsequently assessing the activity, expression, or amount of one or more biomarker in the subject, or in a sample derived from the subject.

In some preferred embodiments the methods comprise subsequently assessing the activity, expression, or amount of Axl in the subject, or in a sample derived from the subject. In some embodiments an increase in the activity, expression, or amount of Axl in the second sample profile as compared with a control profile after contacting the subject with a cancer therapeutic agent is indicative of higher susceptibility to treatment with an agent capable of inhibiting or reversing EMT or a chemotherapeutic agent. In some preferred embodiments the control profile is obtained from the same subject prior to treatment with an agent capable of inhibiting or reversing EMT or a chemotherapeutic agent.

In some preferred embodiments the methods comprise subsequently assessing the activity, expression, or amount of VDBP in the subject, or in a sample derived from the subject. In some embodiments an increase in the activity, expression, or amount of VDBP in the second sample profile as compared with a control profile after contacting the subject with a cancer therapeutic agent is indicative of higher susceptibility to treatment with an agent capable of inhibiting or reversing EMT or a chemotherapeutic agent. In some preferred embodiments the control profile is obtained from the same subject prior to treatment with an agent capable of inhibiting or reversing EMT or a chemotherapeutic agent.

In some preferred embodiments the methods comprise subsequently assessing the activity, expression, or amount of THP in the subject, or in a sample derived from the subject. In some embodiments an increase in the activity, expression, or amount of THP in the second sample profile as compared with a control profile after contacting the subject with a cancer therapeutic agent is indicative of higher susceptibility to treatment with an agent capable of inhibiting or reversing EMT or a chemotherapeutic agent. In some preferred embodiments the control profile is obtained from the same subject prior to treatment with an agent capable of inhibiting or reversing EMT or a chemotherapeutic agent.

In some preferred embodiments the methods comprise subsequently assessing the activity, expression, or amount of EpCam in the subject, or in a sample derived from the subject. In some embodiments an increase in the activity, expression, or amount of EpCam in the second sample profile as compared with a control profile after contacting the subject with a cancer therapeutic agent is indicative of higher susceptibility to treatment with an agent capable of inhibiting or reversing EMT or a chemotherapeutic agent. In some preferred embodiments the control profile is obtained from the same subject prior to treatment with an agent capable of inhibiting or reversing EMT or a chemotherapeutic agent.

Post-Treatment Predictive Biomarkers and Methods

By analysing patient data obtained from clinical trial BGBC003 (NCT02488408), the authors have discovered that, following treatment of subjects having AML or MDS with an agent capable of inhibiting or reversing EMT or a chemotherapeutic agent, the levels of certain of the biomarkers described herein increases in the serum of “responder” subjects but not in the serum of “non-responder” subjects—see Example 1. Similarly, following treatment with an agent capable of inhibiting or reversing EMT or a chemotherapeutic agent, the levels of certain of the biomarkers described herein decreases in the serum of “responder” subjects but not in the serum of “non-responder” subjects. Thus, by assessing the activity, expression, or amount of one or more of the biomarkers described herein prior to and following treatment with an agent capable of inhibiting or reversing EMT or a chemotherapeutic agent it is possible to determine or confirm that the subject is susceptible or responsive to treatment with said agent.

Accordingly, in a second aspect of this facet the disclosure provides a method of predicting a cancer-related outcome in a subject having, suspected of having, or diagnosed with acute myelocytic leukemia (AML) or myelodysplastic syndrome (MDS), the method comprising assessing the activity, expression, or amount of one or more of the biomarkers described herein in a subject, or in a sample derived from the subject after the subject has been contacted with an agent capable of inhibiting or reversing EMT or a chemotherapeutic agent. In some embodiments the cancer-related outcome is susceptibility to treatment with an agent capable of inhibiting or reversing EMT or a chemotherapeutic agent.

In some embodiments the method comprises assessing the activity, expression, or amount of one or more of the biomarkers described herein in the subject, or in a sample derived from the subject, to obtain a sample profile of the one or more biomarker; and, making a prediction based on the sample profile of the one or more biomarker. In some embodiments of the method the prediction is made by comparing the sample profile to a control profile. In preferred embodiments the sample profile is obtained after contacting the subject with an agent capable of inhibiting or reversing EMT or a chemotherapeutic agent.

A “control profile” as used herein is a profile of numerical values or numerical ranges corresponding to the activity, expression, or amount of the same biomarkers as for the sample profile, determined in a control subject or population of control subjects.

In preferred embodiments of this aspect of the disclosure the control profile is obtained from the same subject prior to treatment with an agent capable of inhibiting or reversing EMT or a chemotherapeutic agent. In some embodiments the control profile is obtained from a population of control subjects having AML or MDS. In other embodiments, the control profile is a predetermined profile of biomarker expression, activity, or amount values, for example a profile of “threshold” values or “standard ranges” of values.

Accordingly, in embodiments in which the control profile is obtained from the same subject prior to treatment with an agent capable of inhibiting or reversing EMT or a chemotherapeutic agent, an increase in the activity, expression, or amount of one or more biomarker in the sample profile as compared with a control profile after contacting the subject with an agent capable of inhibiting or reversing EMT or a cancer therapeutic agent may be indicative of susceptibility to treatment with an agent capable of inhibiting or reversing EMT or a chemotherapeutic agent. In such embodiments, a decrease in the activity, expression, or amount of one or more biomarker in the sample profile as compared with a control profile after contacting the subject with a cancer therapeutic agent may be indicative of higher susceptibility to treatment with an agent capable of inhibiting or reversing EMT or a chemotherapeutic agent.

In some such embodiments, an increase in the activity, expression, or amount of one or more biomarker in the sample profile as compared with a control profile after contacting the subject with a cancer therapeutic agent may be indicative of lower susceptibility to treatment with an agent capable of inhibiting or reversing EMT or a chemotherapeutic agent. In some such embodiments, a decrease in the activity, expression, or amount of one or more biomarker in the sample profile as compared with a control profile after contacting the subject with a cancer therapeutic agent may be indicative of lower susceptibility to treatment with an agent capable of inhibiting or reversing EMT or a chemotherapeutic agent.

In some preferred embodiments the one or more biomarker includes at least Axl. In some embodiments the one or more biomarker is Axl. In some embodiments an increase in the activity, expression, or amount of Axl in the sample profile as compared with a control profile after contacting the subject with a cancer therapeutic agent is indicative of higher susceptibility to treatment with an agent capable of inhibiting or reversing EMT or a chemotherapeutic agent.

In some preferred embodiments the one or more biomarker includes at least VDBP. In some embodiments the one or more biomarker is VDBP. In some embodiments an increase in the activity, expression, or amount of VDBP in the sample profile as compared with a control profile after contacting the subject with a cancer therapeutic agent is indicative of higher susceptibility to treatment with an agent capable of inhibiting or reversing EMT or a chemotherapeutic agent.

In some preferred embodiments the one or more biomarker includes at least THP. In some embodiments the one or more biomarker is THP. In some embodiments an increase in the activity, expression, or amount of THP in the sample profile as compared with a control profile after contacting the subject with a cancer therapeutic agent is indicative of higher susceptibility to treatment with an agent capable of inhibiting or reversing EMT or a chemotherapeutic agent.

In some preferred embodiments the one or more biomarker includes at least EpCam. In some embodiments the one or more biomarker is EpCam. In some embodiments an increase in the activity, expression, or amount of EpCam in the sample profile as compared with a control profile after contacting the subject with a cancer therapeutic agent is indicative of higher susceptibility to treatment with an agent capable of inhibiting or reversing EMT or a chemotherapeutic agent.

Subject

A “subject” as referred to herein is any species of animal classified as a mammal, and includes but is not restricted to, domestic and farm animals, primates and humans. In some preferred embodiments the subject is a human of any sex or race. In some embodiments, the human is an adult human.

In some embodiments of this aspect of the disclosure, the subject was selected for treatment with the agent capable of inhibiting or reversing EMT or a chemotherapeutic agent, using a predictive method according to the first aspect of this facet of the disclosure outlined above.

Agents Capable of Inhibiting or Reversing EMT

The present disclosure provides methods for predicting a cancer-related outcome in a subject having, suspected of having, or diagnosed with acute myelocytic leukemia (AML) or myelodysplastic syndrome (MDS). A “cancer-related outcome” is a clinical prediction or prognosis associated with cancer.

The agents capable of inhibiting or reversing EMT for use in this AML or MDS facet of the disclosure are defined as described above in the AML facet (see identically titled section above on page 14, line 19 to page 17, line 7).

Assessing Biomarker Activity, Expression, Amount

In some embodiments of the predictive methods described herein, assessing the activity, expression, or amount of one or more biomarker in a subject, or in a sample derived from a subject, comprises: contacting a sample, or an extract from a sample, with at least 1, 2, 3, 4, 5, 6, 7, 8, 9, 10 or more reagents for detecting the activity, expression, or amount of one or more biomarkers, each of which is selective for a particular biomarker; and, detecting and/or quantifying said reagents for detecting.

In some preferred embodiments, assessing the expression or amount of the one or more biomarker comprises determining the level of the one or more biomarker protein or mRNA in the subject or sample derived from the subject. Preferably the expression or amount of the one or more biomarker is assessed by determining the level of protein expression. In particularly preferred embodiments the expression or amount of the one or more biomarker is assessed by determining the level of protein expression in serum.

The term “expression” refers to the transcription of a gene's DNA template to produce the corresponding mRNA and translation of this mRNA to produce the corresponding gene product (i.e., a peptide, polypeptide, or protein) as well as the “expression” of a protein in one or more forms that may have been modified post translation.

In some embodiments determining the level of the one or more biomarker protein or mRNA comprises: contacting a sample, or an extract from a sample, with at least 1, 2, 3, 4, 5, 6, 7, 8, 9, 10 or more specific binding members, each of which selectively binds to a respective biomarker protein or mRNA; and, detecting and/or quantifying formation of complex formed by said specific binding member and said biomarker protein or mRNA. In some embodiments the specific binding member may comprise an antibody molecule or a binding fragment thereof.

A range of suitable techniques for determining protein and mRNA expression levels are well known in the art, for example microarray analysis, Western blotting, and PCR techniques such as QPCR. In some embodiments, determining the expression or amount of the one or more biomarker protein or mRNA may comprise use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.

In some embodiments, the expression or amount of the one or more biomarker is determined as an absolute amount of the respective biomarker protein or mRNA in the sample. In such cases, the respective biomarker protein or mRNA may be quantified in the sample and the amount expressed as a measure of the sample, for example the volume, mass, or total protein content of the sample.

In some embodiments, the expression or amount of the one or more biomarker is determined as a relative amount of the respective biomarker protein or mRNA in the sample, wherein the relative amount is determined relative to a reference protein or mRNA in the sample. Determining the relative amount of biomarker protein or mRNA in this way allows for normalisation to, e.g., account for differences in total protein concentration and to remove bias from sample to sample.

Preferably the reference protein or mRNA is one whose expression or amount does not vary significantly between subjects having cancer and subjects not having cancer, in particular AML or MDS. That is, the reference protein or mRNA is preferably one whose expression or amount is not altered by AML or MDS.

Accordingly, the reference protein or mRNA may comprise one or more protein or mRNA which is not one of the biomarkers described herein. When expressing the expression or amount of the one or more biomarker as a relative amount, the absolute amount of the respective biomarker protein or mRNA in the sample may be divided by the absolute amount of the reference protein or mRNA in the sample.

Assessing Biomarker Expression at the RNA Level

Gene expression can be detected at the RNA level. RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland). Typical assay formats utilising ribonucleic acid hybridisation include nuclear run-on assays, RT-PCR, RNase protection assays (Melton et al., Nuc. Acids Res. 12:7035), Northern blotting and In Situ hybridization. Gene expression can also be detected by microarray analysis as described below.

Assessing Biomarker Expression at the Protein Level

Preferably biomarker level is assessed by measuring protein expression. Altered gene or protein expression may also be detected by measuring the polypeptides encoded by the gene. This may be achieved by using molecules which bind to the polypeptides encoded by the biomarker gene. Suitable molecules/agents which bind either directly or indirectly to the polypeptides in order to detect the presence of the protein include naturally occurring molecules such as peptides and proteins, for example antibodies, or they may be synthetic molecules.

Antibodies may be derived from commercial sources or through techniques which are familiar to those skilled in the art. In one embodiment, and where altered expression manifests itself through the expression of alteration of post translationally-modified forms of a protein biomarker, antibodies specific for those different forms may be used. For the purposes of this disclosure, the term “antibody”, unless specified to the contrary, includes whole antibodies, or fragments of whole antibodies which retain their binding activity for a target antigen. Such fragments include Fv, F(ab′) and F(ab′)2 fragments, as well as single chain antibodies (scFv). Furthermore, the antibodies and fragments thereof may be humanised antibodies, for example as described in EP239400A. For example: monoclonal and polyclonal antibodies, recombinant antibodies, proteolytic and recombinant fragments of antibodies (Fab, Fv, scFv, diabodies), single-domain antibodies (VHH, sdAb, nanobodies, IgNAR, VNAR), and proteins unrelated to antibodies, which have been engineered to have antibody-like specific binding. Antibodies may be bound to a solid support and/or packaged into kits in a suitable container along with suitable reagents, controls, instructions and the like.

Arrays

Array technology and the various techniques and applications associated with it are well known in the art. Array technology overcomes the disadvantages with traditional methods in molecular biology, which generally work on a “one gene in one experiment” basis, resulting in low throughput and the inability to appreciate the “whole picture” of gene function. In the context of the products and methods of the present disclosure, array technology can be used, for example, in the analysis of the expression of biomarker proteins or mRNA.

In general, any library or group of samples may be arranged in an orderly manner into an array, by spatially separating the members of the library or group. Examples of suitable libraries for arraying include nucleic acid libraries (including DNA, cDNA, oligonucleotide, etc. libraries), peptide, polypeptide and protein libraries, as well as libraries comprising any molecules, such as ligand libraries, among others. Accordingly, where reference is made to a “library” in this document, unless the context dictates otherwise, such reference should be taken to include reference to a library in the form of an array.

Proteins, polypeptides, etc., may also be immobilised in arrays. For example, antibodies have been used in microarray analysis of the proteome using protein chips (Borrebaeck C A, 2000, Immunol Today 21(8):379-82). Polypeptide arrays are reviewed in, for example, MacBeath and Schreiber, 2000, Science, 289(5485):1760-1763.

Sample

Suitable samples include, but are not limited to, tissue samples such as tissue biopsy, blood, urine, buccal scrapes etc., as well as serum, plasma, or tissue culture supernatant samples. In preferred embodiments the expression or amount of the one or more biomarker is assessed by determining the level of protein expression in the sample. In some preferred embodiments the sample is a blood, serum, or plasma sample. In some particularly preferred embodiments the sample is a serum sample.

When assessing the expression or amount of the one or more biomarker proteins or mRNA in serum or plasma samples derived from a subject, samples are removed and subjected to analytical techniques such as flow cytometry, mass cytometry (CyTOF), ELISA, PET, and SELDI-TOF MS. In some embodiments, the method may comprise extracting RNA from said sample and detecting gene expression by QPCR. In some other embodiments, gene expression may be detected by detecting protein products by, for example, Western Blot.

In some embodiments, the level of expression of the one or more biomarker is assessed by determining the copy number of the gene encoding the one or more biomarker. Copy number (i.e. gene duplication events) may be determined using standard techniques known in the art, e.g. using a DNA chip as described in Jiang et al. (Jiang Q, Ho Y Y, Hao L, Nichols Berrios C, Chakravarti A. Copy number variants in candidate genes are genetic modifiers of Hirschsprung disease. PLoS One. 2011; 6(6)).

Is some preferred embodiments of the predictive methods described herein, the method is performed in vitro or ex vivo.

Methods of Selecting a Subject for Treatment

A third aspect of this facet of the present disclosure relates to methods of selecting subjects having, suspected of having, or diagnosed with acute myelocytic leukemia (AML) or myelodysplastic syndrome (MDS) for treatment with an agent capable of inhibiting or reversing EMT or a chemotherapeutic agent. In some embodiments the method comprises: identifying subjects susceptible to treatment with an agent capable of inhibiting or reversing EMT or a chemotherapeutic agent using a predictive method defined in accordance with the above described first and second aspects of this facet of the disclosure; and, selecting thus identified subjects for treatment.

In some embodiments this aspect relates to methods of selecting subjects having, suspected of having, or diagnosed with acute myelocytic leukemia (AML) or myelodysplastic syndrome (MDS) for continued treatment with an agent capable of inhibiting or reversing EMT or a chemotherapeutic agent. In some embodiments the method comprises: identifying subjects susceptible to treatment with an agent capable of inhibiting or reversing EMT or a chemotherapeutic agent using a predictive method defined in accordance with the above described second aspect of this facet of the disclosure; and, selecting thus identified subjects for treatment.

In some embodiments of this aspect of the disclosure, the treatment comprises administering to the subject a therapeutically effective amount of an agent capable of inhibiting or reversing EMT or a chemotherapeutic agent as described herein. Accordingly, in some embodiments the treatment comprises an Axl inhibitor, Akt3 inhibitor, Slfn11 inhibitor, or PHGDH inhibitor. In some preferred embodiments the treatment comprises an Axl inhibitor, for example, BGB324/R428/bemcentinib, cabozantinib, TP-0903, foretinib, merestinib, bosutinib, gilteritinib, crizotinib, amuvatinib, sunitinib, sitravatinib, LDC1267, or UNC2025. Other Axl inhibitors include the anti-Axl antibodies described in WO2015/193428, WO2015/193430, WO2016/097370, and WO2016/166296. In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In some other preferred embodiments, the treatment comprises an Akt3 inhibitor. In some embodiments the Akt3 inhibitor is an Akt3 inhibitor disclosed in WO2016/102672.

In some embodiments of this aspect of the disclosure, the treatment is administered as a single agent. In other embodiments, the treatment is administered in combination with a further cancer treatment. Suitable further cancer treatments are outlined in detail above. In some embodiments the further cancer treatment is a pyrimidine analogue, for example azacitidine, capecitabine, cytarabine, edatrexate, floxuridine, fluorouracil, gemcitabine, or troxacitabine, or a cytidine analogue, for example decitabine. In some particularly preferred embodiments the further cancer treatment is cytarabine. In other particularly preferred embodiments the further cancer treatment is decitabine.

Kits

A fourth aspect of this facet of the present disclosure relates to diagnostic kits and test devices. In some embodiments the disclosure provides a diagnostic kit comprising 1, 2, 3, 4, 5, 6, 7, 8, 9, 10 or more reagents for detecting the activity, expression, or amount of one or more of the biomarkers described herein, each of which is selective for a particular biomarker. In some embodiments each of the reagents for detecting is selective for a biomarker selected from the group consisting of: VDBP, THP, EpCam, EGFR, Fetuin-A, IL-6R beta, Axl, CFHR1, GDF-15, MSP, PON-1, insulin (C-peptide), and/or Apo A-II.

In other embodiments the disclosure provides a test device comprising 1, 2, 3, 4, 5, 6, 7, 8, 9, 10 or more reagents for detecting the activity, expression, or amount of one or more of the biomarkers described herein, each of which is selective for a particular biomarker. In some embodiments each of the reagents for detecting is selective for a biomarker selected from the group consisting of: VDBP, THP, EpCam, EGFR, Fetuin-A, IL-6R beta, Axl, CFHR1, GDF-15, MSP, PON-1, insulin (C-peptide), and/or Apo A-II.

In some embodiments the diagnostic kit or test device comprises 1, 2, 3, 4, 5, 6, 7, 8, 9, 10 or more specific binding members, each of which selectively binds to one of the biomarkers described herein; and, one or more reagents for detecting said one or more specific binding members, or one or more reagents for detecting and/or quantifying formation of a complex formed by said specific binding member and said biomarker. In some embodiments each specific binding member selectively binds to a biomarker selected from the group consisting of: VDBP, THP, EpCam, EGFR, Fetuin-A, IL-6R beta, Axl, CFHR1, GDF-15, MSP, PON-1, insulin (C-peptide), and/or Apo A-II.

In some embodiments the diagnostic kit or test device comprises a specific binding member which selectively binds to Axl, and at least 1, 2, 3, 4, 5, 6, 7, 8, 9, 10, or more specific binding members, each of which selectively binds to a biomarker selected from the group consisting of: VDBP, THP, EpCam, EGFR, Fetuin-A, IL-6R beta, Axl, CFHR1, GDF-15, MSP, PON-1, insulin (C-peptide), and/or Apo A-II.

In some embodiments of this aspect of the disclosure the diagnostic kit or test device comprises a plurality of said specific binding members provided in the form of an array on a substrate or conjugated to a plurality of particles, such as beads or microspheres. The particles may be encoded with a detectable label. In some embodiments the specific binding member comprises an antibody molecule or a binding fragment thereof.

In some embodiments of the diagnostic kit or test device detecting formation of a complex formed by a specific binding member and a biomarker is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.

In some embodiments of the diagnostic kit or test device, the kit or test device comprises no more than 15, 20, 25, 30, 40, or 50 reagents for detecting. In some embodiments of the diagnostic kit or test device, the kit or test device comprises no more than 15, 20, 25, 30, 40, or 50 specific binding members.

The present disclosure also provides use of such diagnostic kits and test devices in methods of predicting a cancer-related outcome in a subject. In preferred embodiments the cancer-related outcome is susceptibility to treatment with an agent capable of inhibiting or reversing EMT or a chemotherapeutic agent as described herein.

In some embodiments the agent capable of inhibiting or reversing EMT or chemotherapeutic agent is an Axl inhibitor, Akt3 inhibitor, Slfn11 inhibitor, or PHGDH inhibitor. In some preferred embodiments the agent capable of inhibiting or reversing EMT or chemotherapeutic agent is an Axl inhibitor, for example BGB324/R428/bemcentinib, cabozantinib, TP-0903, foretinib, merestinib, bosutinib, gilteritinib, crizotinib, amuvatinib, sunitinib, sitravatinib, LDC1267, or UNC2025. Other Axl inhibitors include the anti-Axl antibodies described in WO2015/193428, WO2015/193430, WO2016/097370, and WO2016/166296.

In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In some embodiments of the diagnostic kit or test device for use in a predictive method, the subject has, is suspected of having, or has been diagnosed with AML or MDS.

In some embodiments the method may be as defined in accordance with the above described first and second aspects of this facet of the disclosure.

A fifth aspect of this facet of the present disclosure relates to the use of a reagent for detecting the activity, expression, or amount of a biomarker selected from the group consisting of: VDBP, THP, EpCam, EGFR, Fetuin-A, IL-6R beta, Axl, CFHR1, GDF-15, MSP, PON-1, insulin (C-peptide), and/or Apo A-II, in a method of predicting a cancer-related outcome in a subject.

A sixth aspect of this facet of the disclosure relates to the use of 1, 2, 3, 4, 5, 6, 7, 8, 9, 10 or more reagents for detecting the activity, expression, or amount a biomarker, each of which is selective for a particular biomarker selected from the group consisting of: VDBP, THP, EpCam, EGFR, Fetuin-A, IL-6R beta, Axl, CFHR1, GDF-15, MSP, PON-1, insulin (C-peptide), and/or Apo A-II, in a method of manufacture of a diagnostic kit or test device for use in a method of predicting a cancer-related outcome in a subject. In some embodiments of these uses the reagent for detecting comprises a specific binding member selective for a particular biomarker. The specific binding member may comprise an antibody molecule or a binding fragment thereof.

In some embodiments the method may be as defined in accordance with the above described first and second aspects of this facet of the disclosure.

Uses

A seventh aspect of this facet of the present disclosure relates to the use of one or more of: VDBP, THP, EpCam, EGFR, Fetuin-A, IL-6R beta, Axl, CFHR1, GDF-15, MSP, PON-1, insulin (C-peptide), and/or Apo A-II, as a biomarker for determining if a subject is susceptible to treatment with an agent capable of inhibiting or reversing EMT or a chemotherapeutic agent.

In some embodiments the use relates to use of Axl, and at least 1, 2, 3, 4, 5, 6, 7, 8, 9, 10, or more of: VDBP, THP, EpCam, EGFR, Fetuin-A, IL-6R beta, Axl, CFHR1, GDF-15, MSP, PON-1, insulin (C-peptide), and/or Apo A-II, as a biomarker for determining if a subject is susceptible to treatment with an agent capable of inhibiting or reversing EMT or a chemotherapeutic agent.

In some embodiments the use is as a biomarker in a method as defined in accordance with the above described first and second aspects of this facet of the disclosure.

Methods of Treating a Subject

A eighth aspect of this facet of the present disclosure relates to methods of treating a subject having, suspected of having, or diagnosed with acute myelocytic leukemia (AML) or myelodysplastic syndrome (MDS). In some embodiments the method comprises: identifying a subject for treatment using a predictive method defined in accordance with the above described first and second aspects of this facet of the disclosure; and, administering to the subject a therapeutically effective amount of an agent capable of inhibiting or reversing EMT or a chemotherapeutic agent.

Accordingly, in some embodiments, the method comprises: (a) obtaining a sample from the subject; (b) determining if the subject is susceptible to treatment with an agent capable of inhibiting or reversing EMT or a chemotherapeutic agent using a predictive method defined in accordance with the above described first and aspects of this facet of the disclosure; and, (c) administering to the subject a therapeutically effective amount of an agent capable of inhibiting or reversing EMT or a chemotherapeutic agent.

In some embodiments the method comprises administering a therapeutically effective amount of an agent capable of inhibiting or reversing EMT or chemotherapeutic agent to a subject identified as susceptible to said treatment using a predictive method defined in accordance with the above described first and second aspect of this facets of the disclosure.

In other embodiments the method comprises: (a) administering to a subject a therapeutically effective amount of an agent capable of inhibiting or reversing EMT or a chemotherapeutic agent; and, (b) determining if the subject is susceptible to said treatment using a predictive method defined in accordance with the above described second aspect of this facet of the disclosure. In some embodiments the method further comprises: (c) administering to the subject one or more further therapeutically effective amounts of an agent capable of inhibiting or reversing EMT or a chemotherapeutic agent provided that the subject is identified as susceptible to said treatment.

A ninth aspect of this facet of the present disclosure relates to an agent capable of inhibiting or reversing EMT or chemotherapeutic agent for use in a method of treating a subject having, suspected of having, or diagnosed with acute myelocytic leukemia (AML) or myelodysplastic syndrome (MDS). In some embodiments the method of treatment is a method as defined above.

A tenth aspect of this facet of the present disclosure relates to use of an agent capable of inhibiting or reversing EMT or chemotherapeutic agent for use in a method of manufacture of a medicament for use in a method of treating a subject having, suspected of having, or diagnosed with acute myelocytic leukemia (AML) or myelodysplastic syndrome (MDS). In some embodiments the method of treatment is a method as defined above.

In some embodiments of these methods, agents for use, or uses, the agent capable of inhibiting or reversing EMT or chemotherapeutic agent comprises an Axl inhibitor, Akt3 inhibitor, Slfn11 inhibitor, or PHGDH inhibitor. In some preferred embodiments the agent capable of inhibiting or reversing EMT or chemotherapeutic agent is an Axl inhibitor, for example BGB324/R428/bemcentinib, cabozantinib, TP-0903, foretinib, merestinib, bosutinib, gilteritinib, crizotinib, amuvatinib, sunitinib, sitravatinib, LDC1267, or UNC2025. Other Axl inhibitors include the anti-Axl antibodies described in WO2015/193428, WO2015/193430, WO2016/097370, and WO2016/166296. In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In some other preferred embodiments, the treatment comprises an Akt3 inhibitor. In some embodiments the Akt3 inhibitor is an Akt3 inhibitor disclosed in WO2016/102672.

In some embodiments of these methods, agents for use, or uses, the treatment is administered as a single agent. In other embodiments, the treatment is administered in combination with a further cancer treatment. Suitable further cancer treatments are outlined in detail above. In some embodiments the further cancer treatment is a pyrimidine analogue, for example azacitidine, capecitabine, cytarabine, edatrexate, floxuridine, fluorouracil, gemcitabine, or troxacitabine, or a cytidine analogue, for example decitabine. In some particularly preferred embodiments the further cancer treatment is cytarabine. In other particularly preferred embodiments the further cancer treatment is decitabine.

Statements of Disclosure

The following numbered statements relate to aspects of this facet of the present disclosure, and form part of the description:
101. A method of predicting a cancer-related outcome in a subject, the method comprising assessing the activity, expression, or amount of one or more biomarker in the subject, or in a sample derived from the subject;
   wherein the one or more biomarker is selected from the group consisting of: VDBP, THP, EpCam, EGFR, Fetuin-A, IL-6R beta, Axl, CFHR1, GDF-15, MSP, PON-1, insulin (C-peptide), and/or Apo A-II; and
   wherein the subject has, is suspected of having, or has been diagnosed with acute myelocytic leukemia (AML) or myelodysplastic syndrome (MDS).
102. A method according to statement 101, wherein the method comprises:
   assessing the activity, expression, or amount of one or more biomarker in the subject, or in a sample derived from the subject, to obtain a sample profile of the one or more biomarker; and
   making a prediction based on the sample profile of the one or more biomarker.
103. A method according to statement 102, wherein the prediction is made by comparing the sample profile to a control profile.
104. A method according to statement 103, wherein the sample profile is obtained before the subject is contacted with or administered an agent capable of inhibiting or reversing EMT or a chemotherapeutic agent.
105. A method according to statement 104, wherein the control profile is:
   (i) obtained from a population of control subjects having AML or MDS; or
   (ii) obtained from a control subject or population of control subjects having AML or MDS and previously found to lack susceptibility to treatment with an agent capable of inhibiting or reversing EMT or a chemotherapeutic agent (that is, non-responder subjects);
   (iii) a predetermined profile of biomarker expression, activity, or amount values, for example a profile of “average, median, or mean” values or “standard ranges” of values, obtained from a control subject or population of control subjects having AML or MDS and previously found to lack susceptibility to treatment with an agent capable of inhibiting or reversing EMT or a chemotherapeutic agent (that is, non-responder subjects);
   (iv) obtained from a control sample having a known “average, median, or mean” value of biomarkers indicative of a control subject or population of control subjects having AML or MDS and previously found to lack susceptibility to treatment with an agent capable of inhibiting or reversing EMT or a chemotherapeutic agent (that is, non-responder subjects);
   (v) a predetermined profile of biomarker expression, activity, or amount “threshold” values obtained from a control subject or population of control subjects having AML or MDS and previously found to be susceptible to treatment with an agent capable of inhibiting or reversing EMT or a chemotherapeutic agent (that is, responder subjects); or
   (vi) obtained from a control sample having a known “threshold” value of biomarkers indicative of a control subject or population of control subjects having AML or MDS and previously found to be susceptible to treatment with an agent capable of inhibiting or reversing EMT or a chemotherapeutic agent (that is, responder subjects).
106. A method according to statement 105, wherein a higher activity, expression, or amount of one or more biomarker in the sample profile as compared with the control profile is indicative of susceptibility to treatment with an agent capable of inhibiting or reversing EMT or a chemotherapeutic agent.
107. A method according to statement 105, wherein a lower activity, expression, or amount of one or more biomarker in the sample profile as compared with the control profile is indicative of susceptibility to treatment with an agent capable of inhibiting or reversing EMT or a chemotherapeutic agent.
108. A method according to any one of statements 105-107, wherein the one or more biomarker includes at least one of: VDBP, THP, EpCam, EGFR, Fetuin-A, IL-6R beta, MSP, PON-1, and/or Apo A-II,
   and wherein a higher activity, expression, or amount of one or more of said biomarkers in the sample profile as compared with the control profile is indicative of susceptibility to treatment with an agent capable of inhibiting or reversing EMT or a chemotherapeutic agent.
109. A method according to any one of statements 105-108, wherein the one or more biomarker includes at least one of: Axl, GDF-15, Insulin (c-peptide) and/or CFHR1,
   and wherein a lower activity, expression, or amount of one or more of said biomarkers in the sample profile as compared with the control profile is indicative of susceptibility to treatment with an agent capable of inhibiting or reversing EMT or a chemotherapeutic agent.
110. A method according to any one of statements 105-109, wherein:
   a lower activity, expression, or amount of Axl; and/or
   a lower activity, expression, or amount of GDF-15; and/or
   a lower activity, expression, or amount of Insulin (c-peptide); and/or
   a lower activity, expression, or amount of CHFR1; and/or
   a higher activity, expression, or amount of THP; and/or
   a higher activity, expression, or amount of VDBP; and/or
   a higher activity, expression, or amount of EpCam; and/or
   a higher activity, expression, or amount of EGFR; and/or
   a higher activity, expression, or amount of Fetuin-A; and/or
   a higher activity, expression, or amount of IL-6R beta; and/or
   a higher activity, expression, or amount of MSP; and/or
   a higher activity, expression, or amount of PON-1; and/or
   a higher activity, expression, or amount of Apo-AII;
   in the sample profile as compared with the control profile is indicative of susceptibility to treatment with an agent capable of inhibiting or reversing EMT or a chemotherapeutic agent.
111. A method according to any one of statements 101-110, wherein the one or more biomarker comprises Axl,
   optionally wherein the one or more biomarker comprises Axl and at least 1, 2, 3, 4, 5, 6, 7, 8, 9, 10, or more further biomarkers selected from: VDBP, THP, EpCam, EGFR, Fetuin-A, IL-6R beta, CFHR1, GDF-15, MSP, PON-1, insulin (C-peptide), and/or Apo A-II.
112. A method according to any one of statements 101-110, wherein the one or more biomarker comprises VDBP,
   optionally wherein the one or more biomarker comprises VDBP and at least 1, 2, 3, 4, 5, 6, 7, 8, 9, 10, or more further biomarkers selected from: THP, EpCam, EGFR, Fetuin-A, IL-6R beta, Axl, CFHR1, GDF-15, MSP, PON-1, insulin (C-peptide), and/or Apo A-II.
113. A method according to any one of statements 101-110, wherein the one or more biomarker comprises THP,
   optionally wherein the one or more biomarker comprises THP and at least 1, 2, 3, 4, 5, 6, 7, 8, 9, 10, or more further biomarkers selected from: VDBP, EpCam, EGFR, Fetuin-A, IL-6R beta, Axl, CFHR1, GDF-15, MSP, PON-1, insulin (C-peptide), and/or Apo A-II.
114. A method according to any one of statements 101-110, wherein the one or more biomarker comprises EpCam,
   optionally wherein the one or more biomarker comprises EpCam and at least 1, 2, 3, 4, 5, 6, 7, 8, 9, 10, or more further biomarkers selected from: VDBP, THP, EGFR, Fetuin-A, IL-6R beta, Axl, CFHR1, GDF-15, MSP, PON-1, insulin (C-peptide), and/or Apo A-II.
115. A method according to any one of statements 101-110, wherein the one or more biomarker comprises VDBP and THP,
   optionally wherein the one or more biomarker comprises VDBP, THP, and at least 1, 2, 3, 4, 5, 6, 7, 8, 9, 10, or more further biomarkers selected from: EpCam, EGFR, Fetuin-A, IL-6R beta, Axl, CFHR1, GDF-15, MSP, PON-1, insulin (C-peptide), and/or Apo A-II.
116. A method according to any one of statements 101-110, wherein the one or more biomarker comprises VDBP and EpCam,
   optionally wherein the one or more biomarker comprises VDBP, EpCam, and at least 1, 2, 3, 4, 5, 6, 7, 8, 9, 10, or more further biomarkers selected from: THP, EGFR, Fetuin-A, IL-6R beta, Axl, CFHR1, GDF-15, MSP, PON-1, insulin (C-peptide), and/or Apo A-II.
117. A method according to any one of statements 101-110, wherein the one or more biomarker comprises EpCam and THP,
   optionally wherein the one or more biomarker comprises EpCam, THP, and at least 1, 2, 3, 4, 5, 6, 7, 8, 9, 10, or more further biomarkers selected from: VDBP, EGFR, Fetuin-A, IL-6R beta, Axl, CFHR1, GDF-15, MSP, PON-1, insulin (C-peptide), and/or Apo A-II.
118. A method according to any one of statements 101-110, wherein the one or more biomarker comprises VDBP, THP, and EpCam,
   optionally wherein the one or more biomarker comprises VDBP, THP, EpCam, and at least 1, 2, 3, 4, 5, 6, 7, 8, 9, 10, or more further biomarkers selected from: EGFR, Fetuin-A, IL-6R beta, Axl, CFHR1, GDF-15, MSP, PON-1, insulin (C-peptide), and/or Apo A-II.
119. A method according to any one of statements 112-118, wherein the one or more biomarker further comprises Axl.
120. A method according to any one of statements 101-119, wherein:
   the Axl biomarker comprises an amino acid sequence having at least 70%, 80%, 90%, 95%, 99% or 100% sequence identity with the full-length of the amino acid sequence disclosed at UniProt accession no: P30530-1 or P30530-2 (entry version 196) or a fragment thereof;
   the Tamm-Horsfall Urinary Glycoprotein (THP) biomarker comprises an amino acid sequence having at least 70%, 80%, 90%, 95%, 99% or 100% sequence identity with the full-length of the amino acid sequence disclosed at UniProt accession no: P07911-1, P07911-2, P07911-3, P07911-4, or P07911-5 (entry version 182) or a fragment thereof;
   the Vitamin D-Binding Protein (VDBP) biomarker comprises an amino acid sequence having at least 70%, 80%, 90%, 95%, 99% or 100% sequence identity with the full-length of the amino acid sequence disclosed at UniProt accession no: P02774-1, P02774-2, or P02774-3 (entry version 201) or a fragment thereof;
   the Epithelial cell adhesion molecule (EpCam) biomarker comprises an amino acid sequence having at least 70%, 80%, 90%, 95%, 99% or 100% sequence identity with the full-length of the amino acid sequence disclosed at UniProt accession no: P16422-1 (entry version 186) or a fragment thereof;
   the Epidermal Growth Factor Receptor (EGFR) biomarker comprises an amino acid sequence having at least 70%, 80%, 90%, 95%, 99% or 100% sequence identity with the full-length of the amino acid sequence disclosed at UniProt accession no: Q504U8 (entry version 138) or a fragment thereof;
   the Fetuin-A biomarker comprises an amino acid sequence having at least 70%, 80%, 90%, 95%, 99% or 100% sequence identity with the full-length of the amino acid sequence disclosed at UniProt accession no: P02765-1 (entry version 205) or a fragment thereof;
   the Interleukin-6 receptor subunit beta (IL-6R beta) biomarker comprises an amino acid sequence having at least 70%, 80%, 90%, 95%, 99% or 100% sequence identity with the full-length of the amino acid sequence disclosed at UniProt accession no: P08887-1 or P08887-2 (entry version 210) or a fragment thereof;
   the Complement Factor H-Related Protein 1 (CFHR1) biomarker comprises an amino acid sequence having at least 70%, 80%, 90%, 95%, 99% or 100% sequence identity with the full-length of the amino acid sequence disclosed at UniProt accession no: Q03591-1 (entry version 162) or a fragment thereof;
   the Growth/differentiation factor 15 (GDF-15) biomarker comprises an amino acid sequence having at least 70%, 80%, 90%, 95%, 99% or 100% sequence identity with the full-length of the amino acid sequence disclosed at UniProt accession no: Q03591-1 (entry version 165) or a fragment thereof;
   the Macrophage-Stimulating Protein (MSP) biomarker comprises an amino acid sequence having at least 70%, 80%, 90%, 95%, 99% or 100% sequence identity with the full-length of the amino acid sequence disclosed at UniProt accession no: P26927 (entry version 179) or a fragment thereof;
   the Paraoxonase-1 (PON-1) biomarker comprises an amino acid sequence having at least 70%, 80%, 90%, 95%, 99% or 100% sequence identity with the full-length of the amino acid sequence disclosed at UniProt accession no: P27169-1 (entry version 202) or a fragment thereof;
   the insulin (C-peptide) biomarker comprises an amino acid sequence having at least 70%, 80%, 90%, 95%, 99% or 100% sequence identity with the full-length of the amino acid sequence disclosed at UniProt accession no: P01308-1 (entry version 245), F8WCM5-1 (entry version 57) or a fragment thereof; and/or
   the Apolipoprotein A-II (Apo A-II) biomarker comprises an amino acid sequence having at least 70%, 80%, 90%, 95%, 99% or 100% sequence identity with the full-length of the amino acid sequence disclosed at UniProt accession no: P01871-1 or P01871-2 (entry version 180) or a fragment thereof.
121. A method according to any one of statement 101-120, wherein the method comprises determining the amount of at least 2, 3, 4, 5, 6, 7, 8, 9, 10, 11, 12, 13, 14, 15, 20 of said biomarkers in the subject, or sample derived from the subject.
122. A method according to any one of statements 104-121, wherein assessing the activity, expression, or amount of one or more biomarker in the subject, or in a sample derived from the subject, comprises:
   contacting a sample, or an extract from the sample, with at least 1, 2, 3, 4, 5, 6, 7, 8, 9, 10 or more reagents for detecting the activity, expression, or amount of one or more biomarkers, each of which is selective for a particular biomarker; and
   detecting and/or quantifying said reagents for detecting.
123. A method according to statement 122, wherein assessing the expression or amount of one or more biomarker in the subject, or in a sample derived from the subject, comprises:
   contacting a sample, or an extract from the sample, with at least 1, 2, 3, 4, 5, 6, 7, 8, 9, 10 or more specific binding members, each of which selectively binds to a respective biomarker protein or mRNA; and
   detecting and/or quantifying formation of complex formed by said specific binding member and said biomarker protein or mRNA.
124. A method according to statement 123 wherein the specific binding member comprises an antibody molecule or binding fragment thereof.
125. A method according to statement 123 or 124, wherein detecting formation of a complex formed by the specific binding member and the biomarker protein or mRNA is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.
126. A method according to any one of statements 123-125 wherein the expression or amount of the one or more biomarker is determined as the absolute amount of the respective biomarker protein or mRNA in the sample.
127. A method according to any one of statements 123-125 wherein the expression or amount of the one or more biomarker is determined as a relative amount of the respective biomarker protein or mRNA in the sample,
   optionally wherein the relative amount is determined relative to a reference protein or mRNA in the sample, the expression of which is not altered by AML or MDS.
128. A method according to any one of statements 101-127, wherein the cancer-related outcome is susceptibility to treatment with an agent capable of inhibiting or reversing EMT or a chemotherapeutic agent.
129. A method according to statement 128, wherein the agent capable of inhibiting or reversing EMT or chemotherapeutic agent is an Axl inhibitor, Akt3 inhibitor, Slfn11 inhibitor, or PHGDH inhibitor.
130. A method according to statement 129, wherein the agent capable of inhibiting or reversing EMT or chemotherapeutic agent is an Axl inhibitor.
131. A method according to statement 130, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
132. A method according to statement 131, wherein the Axl inhibitor is BGB324/R428/bemcentinib.
133. A method according to statement 131, wherein the Axl inhibitor is an anti-Axl antibody described in WO2015/193428, WO2015/193430, WO2016/097370, or WO2016/166296.
134. A method according to statement 129, wherein the agent capable of inhibiting or reversing EMT or chemotherapeutic agent is an Akt3 inhibitor.
135. A method according to statement 134, wherein the Akt3 inhibitor is an Akt3 inhibitor disclosed in WO2016/102672.
136. A method according to any one of statements 128-135, wherein the agent capable of inhibiting or reversing EMT is administered as a single agent.
137. A method according to any one of statements 128-135, wherein the agent capable of inhibiting or reversing EMT is administered in combination with a further cancer treatment.
138. A method according to statement 137, wherein the further cancer treatment is selected from:
   (i) alkylating agents, including alkyl sulfonates such as busulfan;
   (ii) nitrogen mustards such as chlorambucil, cyclophosphamide, estramustine, ifosfamide, mechlorethamine, melphalan, and uramustine;
   (iii) ethyleneimine derivatives such as thiotepa;
   (iv) nitrosoureas such as carmustine, lomustine, and streptozocin;
   (v) triazenes such as dacarbazine, procarbazine, and temozolamide;
   (vi) platinum compounds such as cisplatin, carboplatin, oxaliplatin, satraplatin, and picoplatin onnaplatin, tetraplatin, sprioplatin, iproplatin, chloro(diethylenediamino)-platinum (II) chloride, dichloro(ethylenediamino)-platinum (II), diamino(2-ethylmalonato)platinum (II), (1,2-diaminocyclohexane)malonatoplatinum (II), (4-carboxyphthalo)-(1,2-diaminocyclohexane)platinum (II), (1,2-diaminocyclohexane)-(isocitrato)platinum (II), and (1,2-diaminocyclohexane)-cis-(pyruvato)platinum (II);
   (vii) antimetabolites, including antifolates such as methotrexate, permetrexed, raltitrexed, and trimetrexate;
   (viii) pyrimidine analogues such as azacitidine, capecitabine, cytarabine, edatrexate, floxuridine, fluorouracil, gemcitabine, and troxacitabine;
   (ix) purine analogues such as cladribine, chlorodeoxyadenosine, clofarabine, fludarabine, mercaptopurine, pentostatin, and thioguanine;
   (x) natural products, including antitumor antibiotics such as bleomycin, dactinomycin, mithramycin, mitomycin, mitoxantrone, porfiromycin;
   (xi) anthracyclines such as daunorubicin, doxorubicin, epirubicin, idarubicin, and valrubicin;
   (xii) mitotic inhibitors such as the vinca alkaloids vinblastine, vinvesir, vincristine, vindesine, and vinorelbine;
   (xiii) enzymes such as L-asparaginase and PEG-L-asparaginase;
   (xiv) microtubule polymer stabilizers such as the taxanes paclitaxel and docetaxel;
   (xv) topoisomerase I inhibitors such as the camptothecins irinotecan and topotecan;
   (xvi) topoisomerase II inhibitors such as podophyllotoxin, amsacrine, etoposide, teniposide, losoxantrone and actinomycin;
   (xvii) hormones and hormone antagonists, including
      androgens such as fluoxymesterone and testolactone;
      antiandrogens such as bicalutamide, cyproterone, flutamide, and nilutamide;
      corticosteroids such as dexamethasone and prednisone;
      aromatase inhibitors such as aminoglutethimide, anastrozole, exemestane, formestane, and letrozole;
      estrogens such as diethylstilbestrol;
      antiestrogens such as fulvestrant, raloxifene, tamoxifen, and toremifine;
      luteinising hormone-releasing hormone (LHRH) agonists and antagonists such as abarelix, buserelin, goserelin, leuprolide, histrelin, desorelin, nafarelin acetate and triptorelin;
      progestins such as medroxyprogesterone acetate and megestrol acetate; and
      thyroid hormones such as levothyroxine and liothyronine;
   (xviii) PKB pathway inhibitors, including perifosine, enzastaurin hydrochloride, and triciribine;
   (xix) P13K inhibitors such as semaphore and SF1126, and MTOR inhibitors such as rapamycin and analogues;
   (xx) CDK inhibitors, including seliciclib, alvocidib, and 7-hydroxystaurosporine;
   (xxi) COX-2 inhibitors, including celecoxib;
   (xxii) HDAC inhibitiors, including trichostatin A, suberoylanilide hydroxamic acid, and chlamydocin;
   (xxiii) DNA methylase inhibitors, including temozolomide, and miscellaneous agents, including altretamine, arsenic trioxide, thalidomide, lenalidomide, gallium nitrate, levamisole, mitotane, hydroxyurea, octreotide, procarbazine, suramin, photodynamic compounds such as methoxsalen and sodium porfimer;
   (xxiv) proteasome inhibitors such as bortezomib;
   (xxv) molecular targeted therapy agents including: functional therapeutic agents, including gene therapy agents and antisense therapy agents;
   (xxvi) tyrosine kinase inhibitors such as erlotinib hydrochloride, gefitinib, imatinib mesylate, and semaxanib;
   (xxvii) Raf inhibitors such as sorafenib, and gene expression modulators such as the retinoids and rexinoids, for example adapalene, bexarotene, trans-retinoic acid, 9-cis-retinoic acid, and N-(4-hydroxyphenyl)retinamide;
   (xxviii) phenotype-directed therapy agents, including monoclonal antibodies such as pembrolizumab, alemtuzumab, bevacizumab, cetuximab, ibritumomab tiuxetan, rituximab, and trastuzumab, immunotoxins such as gemtuzumab ozogamicin, radioimmunoconjugates such as I-tositumobab;
   (xxix) cancer vaccines;
   (xxx) biologic therapy agents including: interferons such as interferon-[alpha]2a and interferon-[alpha]2b, and interleukins such as aldesleukin, denileukin diftitox, and oprelvekin;
   (xxxi) anticancer therapies involving the use of protective or adjunctive agents, including: cytoprotective agents such as amifostine, and dexrazoxane, phosphonates such as pamidronate and zoledronic acid, and stimulating factors such as epoetin, darbeopetin, filgrastim, PEG-filgrastim, and sargramostim;
   (xxxii) Axl inhibitors such as 1-(6,7-dihydro-5H-benzo[6,7]cyclohepta[1,2-c]pyridazin-3-yl)-N3-((7-(S)-pyrrolidin-1-yl)-6,7,8,9-tetrahydro-5H-benzo[7]annulene-2-yl)-1H-1,2,4-triazole-3,5-diamine; and/or
   (xxxiii) further combination chemotherapeutic regimens, such as combinations of carboplatin/paclitaxel, capecitabine/docetaxel, fluorauracil/levamisole, fluorauracil/leucovorin, methotrexate/leucovorin, and trastuzumab/paclitaxel, alone or in further combination with carboplatin.
139. A method according to statement 137, wherein the further cancer treatment is a pyrimidine analogue, for example azacitidine, capecitabine, cytarabine, edatrexate, floxuridine, fluorouracil, gemcitabine, or troxacitabine, or a cytidine analogue, for example decitabine.
140. A method according to statement 139, wherein the further cancer treatment is cytarabine.
141. A method according to statement 139, wherein the further cancer treatment is decitabine.
142. A method according to any one of statements 101-141, wherein the subject is mammalian.
143. A method according to statement 142, wherein the subject is human.
144. A method according to any one of statements 101-143, wherein the predictive method is performed before the subject receives a treatment or course of treatment for AML or MDS.
145. A method according to any one of statement 101-144, wherein the predictive method is performed during or after a treatment or course of treatment for AML or MDS.
146. A method according to any one of statements 101-143 or 145, wherein the subject has previously been treated with an agent capable of inhibiting or reversing EMT or a chemotherapeutic agent.
147. A method according to statement 146, wherein the subject has previously been treated with an agent selected from: a PHGDH inhibitor, a Slfn11 inhibitor, an Axl inhibitor, or an Akt3 inhibitor.
148. A method according to any one of statements 146-147, wherein the subject has previously been treated with an agent that is different to the agent capable of inhibiting or reversing EMT or chemotherapeutic agent for which the subject's susceptibility is being predicted.
149. A method according to any one of statements 101-144, wherein the subject has not previously been treated with an agent capable of inhibiting or reversing EMT or a chemotherapeutic agent.
150. A method according to statement 149, wherein the subject has not previously been treated with an Axl inhibitor.
151. A method according to statement 149, wherein the subject has not previously been treated with an Akt3 inhibitor.
152. A method according to any one of statements 101-151, wherein the sample is selected from: blood, serum, plasma, tissue biopsy, culture supernatant, or urine.
153. A method according to statement 152, wherein the sample is a blood, serum, or plasma sample.
154. A method according to statement 153, wherein the sample is a serum sample.
155. A method according to any one of statements 104-154, wherein the method further comprises a subsequent step of assessing the activity, expression, or amount of one or more biomarker in the subject, in which a sample profile is obtained after contacting the subject with an agent capable of inhibiting or reversing EMT or a chemotherapeutic agent.
156. A method according to statement 103, wherein the sample profile is obtained after contacting the subject with an agent capable of inhibiting or reversing EMT or a chemotherapeutic agent.
157. A method according to statement 155 or 156, wherein the control profile is obtained from the same subject prior to contacting said subject with the agent capable of inhibiting or reversing EMT or a chemotherapeutic agent.
158. A method according to statement 157, wherein an increase in the activity, expression, or amount of one or more biomarker in the sample profile as compared with the control profile is indicative of higher susceptibility to treatment with an agent capable of inhibiting or reversing EMT or a chemotherapeutic agent.
159. A method according to statement 157, wherein a decrease in the activity, expression, or amount of one or more biomarker in the sample profile as compared with the control profile is indicative of higher susceptibility to treatment with an agent capable of inhibiting or reversing EMT or a chemotherapeutic agent.
160. A method according to statement 157, wherein an increase in the activity, expression, or amount of one or more biomarker in the sample profile as compared with the control profile is indicative of lower susceptibility to treatment with an agent capable of inhibiting or reversing EMT or a chemotherapeutic agent.
161. A method according to statement 157, wherein a decrease in the activity, expression, or amount of one or more biomarker in the sample profile as compared with the control profile is indicative of lower susceptibility to treatment with an agent capable of inhibiting or reversing EMT or a chemotherapeutic agent.
162. A method according to statement 158, wherein the one or more biomarker includes at least Axl.
163. A method according to statement 158, wherein the one or more biomarker includes at least VDBP.
164. A method according to statement 158, wherein the one or more biomarker includes at least THP.
165. A method according to statement 158, wherein the one or more biomarker includes at least EpCam.
166. A method according to statement 158, wherein the one or more biomarker includes at least VDBP and THP.
167. A method according to statement 158, wherein the one or more biomarker includes at least VDBP and EpCam.
168. A method according to statement 158, wherein the one or more biomarker includes at least THP and EpCam
169. A method according to statement 158, wherein the one or more biomarker includes at least VDBP, THP, and EpCam.
170. A method according to any one of statements 163-169, wherein the one or more biomarker further includes Axl.
171. A method according to any one of statements 155-170, wherein assessing the activity, expression, or amount of one or more biomarker in the subject, or in a sample derived from the subject, comprises:
   contacting a sample, or an extract from the sample, with at least 1, 2, 3, 4, 5, 6, 7, 8, 9, 10 or more reagents for detecting the activity, expression, or amount of one or more biomarkers, each of which is selective for a particular biomarker; and
   detecting and/or quantifying said reagents for detecting.
172. A method according to statement 171, wherein assessing the expression or amount of one or more biomarker in the subject, or in a sample derived from the subject, comprises:
   contacting a sample, or an extract from the sample, with at least 1, 2, 3, 4, 5, 6, 7, 8, 9, 10 or more specific binding members, each of which selectively binds to a respective biomarker protein or mRNA; and
   detecting and/or quantifying formation of a complex formed by said specific binding member and said biomarker protein or mRNA.
173. A method according to statement 172, wherein the specific binding member comprises an antibody molecule or binding fragment thereof.
174. A method according to statement 172 or 173, wherein detecting formation of a complex formed by the specific binding member and the biomarker protein or mRNA is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.
175. A method according to any one of statements 172-174 wherein the expression or amount of the one or more biomarker is determined as the absolute amount of the respective biomarker protein or mRNA in the sample.
176. A method according to any one of statements 172-174 wherein the expression or amount of the one or more biomarker is determined as a relative amount of the respective biomarker protein or mRNA in the sample,
   optionally wherein the relative amount is determined relative to a reference protein or mRNA in the sample, the expression of which is not altered by AML or MDS.
177. A method according to any one of statements 156-176, wherein the agent capable of inhibiting or reversing EMT or chemotherapeutic agent is an Axl inhibitor, Akt3 inhibitor, Slfn11 inhibitor, or PHGDH inhibitor.
178. A method according to statement 177, wherein the agent capable of inhibiting or reversing EMT or chemotherapeutic agent is an Axl inhibitor.
179. A method according to statement 178, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
180. A method according to statement 179, wherein the Axl inhibitor is BGB324/R428/bemcentinib.
181. A method according to statement 179, wherein the Axl inhibitor is an anti-Axl antibody described in WO2015/193428, WO2015/193430, WO2016/097370, or WO2016/166296.
182. A method according to statement 177, wherein the agent capable of inhibiting or reversing EMT or chemotherapeutic agent is an Akt3 inhibitor.
183. A method according to statement 182, wherein the Akt3 inhibitor is an Akt3 inhibitor disclosed in WO2016/102672.
184. A method according to any one of statements 177-183, wherein the agent capable of inhibiting or reversing EMT is administered as a single agent.
185. A method according to any one of statements 177-183, wherein the agent capable of inhibiting or reversing EMT is administered in combination with a further cancer treatment.
186. A method according to statement 185, wherein the further cancer treatment is selected from the further cancer treatments listed in statement 138.
187. A method according to statement 185, wherein the further cancer treatment is a pyrimidine analogue, for example azacitidine, capecitabine, cytarabine, edatrexate, floxuridine, fluorouracil, gemcitabine, or troxacitabine, or a cytidine analogue, for example decitabine.
188. A method according to statement 187, wherein the further cancer treatment is cytarabine.
189. A method according to statement 187, wherein the further cancer treatment is decitabine.
190. A method according to any one of statements 156-189, wherein the subject is mammalian.
191. A method according to statement 190, wherein the subject is human.
192. A method according to any one of statements 156-191, wherein the subject was selected for treatment with the agent capable of inhibiting or reversing EMT or a chemotherapeutic agent, using a predictive method according to any one of statements 101-155.
193. A method according to any one of statements 156-192, wherein the sample is selected from: blood, serum, plasma, tissue biopsy, culture supernatant, or urine.
194. A method according to statement 193, wherein the sample is a blood, serum, or plasma sample.
195. A method according to statement 194, wherein the sample is a serum sample.
196. A method according to any one of statements 1-195, wherein the method is performed in vitro or ex vivo.
201. A method of selecting subjects having, suspected of having, or diagnosed with acute myelocytic leukemia (AML) or myelodysplastic syndrome (MDS) for treatment with an agent capable of inhibiting or reversing EMT or a chemotherapeutic agent, the method comprising:
   identifying subjects susceptible to treatment with an agent capable of inhibiting or reversing EMT or a chemotherapeutic agent using a method according to any one of statements 101-196; and
   selecting thus identified subjects for treatment.
202. A method of selecting subjects having, suspected of having, or diagnosed with acute myelocytic leukemia (AML) or myelodysplastic syndrome (MDS) for continued treatment with an agent capable of inhibiting or reversing EMT or a chemotherapeutic agent, the method comprising:
   identifying subjects susceptible to treatment with an agent capable of inhibiting or reversing EMT or a chemotherapeutic agent using a method according to any one of statements 156-196; and
   selecting thus identified subjects for treatment.
203. A method according to any one of statements 201-202, wherein treatment comprises administering to the subject a therapeutically effective amount of an agent capable of inhibiting or reversing EMT or a chemotherapeutic agent.
204. A method according to statement 203, wherein the treatment comprises an Axl inhibitor, Akt3 inhibitor, Slfn11 inhibitor, or PHGDH inhibitor.
205. A method according to statement 204, wherein the treatment comprises an Axl inhibitor.
206. A method according to statement 205, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
207. A method according to statement 206, wherein the Axl inhibitor is BGB324/R428/bemcentinib.
208. A method according to statement 206, wherein the Axl inhibitor is an anti-Axl antibody described in WO2015/193428, WO2015/193430, WO2016/097370, or WO2016/166296.
209. A method according to statement 204, wherein the treatment comprises an Akt3 inhibitor.
210. A method according to statement 209, wherein the Akt3 inhibitor is an Akt3 inhibitor disclosed in WO2016/102672.
211. A method according to any one of statements 203-210, wherein the treatment is administered as a single agent.
212. A method according to any one of statements 203-211, wherein the treatment is administered in combination with a further cancer treatment.
213. A method according to statement 212, wherein the further cancer treatment is selected from the further cancer treatments listed in statement 138.
214. A method according to statement 212, wherein the further cancer treatment is a pyrimidine analogue, for example azacitidine, capecitabine, cytarabine, edatrexate, floxuridine, fluorouracil, gemcitabine, or troxacitabine, or a cytidine analogue, for example decitabine.
215. A method according to statement 214, wherein the further cancer treatment is cytarabine.
216. A method according to statement 214, wherein the further cancer treatment is decitabine.
301. A diagnostic kit comprising 1, 2, 3, 4, 5, 6, 7, 8, 9, 10 or more reagents for detecting the activity, expression, or amount of one or more biomarkers, each of which is selective for a particular biomarker selected from the group consisting of: VDBP, THP, EpCam, EGFR, Fetuin-A, IL-6R beta, Axl, CFHR1, GDF-15, MSP, PON-1, insulin (C-peptide), and/or Apo A-II.
302. A test device comprising 1, 2, 3, 4, 5, 6, 7, 8, 9, 10 or more reagents for detecting the activity, expression, or amount of one or more biomarkers, each of which is selective for a particular biomarker selected from the group consisting of: VDBP, THP, EpCam, EGFR, Fetuin-A, IL-6R beta, Axl, CFHR1, GDF-15, MSP, PON-1, insulin (C-peptide), and/or Apo A-II.
303. A diagnostic kit or test device according to any one of statements 301 or 302, comprising:
1, 2, 3, 4, 5, 6, 7, 8, 9, 10 or more specific binding members, each of which selectively binds to a biomarker selected from the group consisting of: VDBP, THP, EpCam, EGFR, Fetuin-A, IL-6R beta, Axl, CFHR1, GDF-15, MSP, PON-1, insulin (C-peptide), and/or Apo A-II; and
   one or more reagents for detecting said one or more specific binding members or one or more reagents for detecting and/or quantifying formation of a complex formed by said specific binding member and said biomarker.
304. A diagnostic kit or test device according to statement 303, wherein the specific binding member comprises an antibody molecule or binding fragment thereof.
305. A diagnostic kit or test device according to statement 303 or 304, wherein detecting formation of a complex formed by the specific binding member and the biomarker is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.
306. A diagnostic kit or test device according to any one of statements 301-305, wherein the kit or test device comprises no more than 15, 20, 25, 30, 40, or 50 reagents for detecting.
307. A diagnostic kit or test device according to any one of statements 301-306 for use in a method of predicting a cancer-related outcome in a subject.
308. A diagnostic kit for use, or test device for use, according to statement 307, wherein the cancer-related outcome is susceptibility to treatment with an agent capable of inhibiting or reversing EMT or a chemotherapeutic agent.
309. A diagnostic kit for use, or test device for use, according to statement 308, wherein the agent capable of inhibiting or reversing EMT or chemotherapeutic agent is an Axl inhibitor, Akt3 inhibitor, Slfn11 inhibitor, or PHGDH inhibitor.
310. A diagnostic kit for use, or test device for use, according to statement 308, wherein the agent capable of inhibiting or reversing EMT or chemotherapeutic agent is an Axl inhibitor.
311. A diagnostic kit for use, or test device for use, according to statement 310, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
312. A diagnostic kit for use, or test device for use, according to statement 311, wherein the Axl inhibitor is BGB324/R428/bemcentinib.
313. A diagnostic kit for use, or test device for use, according to statement 311, wherein the Axl inhibitor is an anti-Axl antibody described in WO2015/193428, WO2015/193430, WO2016/097370, or WO2016/166296.
314. A diagnostic kit for use, or test device for use, according to statement 308, wherein the agent capable of inhibiting or reversing EMT or chemotherapeutic agent is an Akt3 inhibitor.
315. A diagnostic kit for use, or test device for use, according to any one of statements 307-310, wherein the subject has, is suspected of having, or has been diagnosed with acute myelocytic leukemia (AML) or myelodysplastic syndrome (MDS).
316. A diagnostic kit for use, or test device for use, according to any one of statements 307-315, wherein the method is a method according to any one of statements 101-196.
317. Use of a reagent for detecting the activity, expression, or amount of a biomarker selected from the group consisting of: VDBP, THP, EpCam, EGFR, Fetuin-A, IL-6R beta, Axl, CFHR1, GDF-15, MSP, PON-1, insulin (C-peptide), and/or Apo A-II, in a method of predicting a cancer-related outcome in a subject.
318. Use of 1, 2, 3, 4, 5, 6, 7, 8, 9, 10 or more reagents for detecting the activity, expression, or amount a biomarker, each of which is selective for a particular biomarker selected from the group consisting of: VDBP, THP, EpCam, EGFR, Fetuin-A, IL-6R beta, Axl, CFHR1, GDF-15, MSP, PON-1, insulin (C-peptide), and/or Apo A-II, in a method of manufacture of a diagnostic kit or test device for use in a method of predicting a cancer-related outcome in a subject.
319. A use according to statement 317 or 318, wherein the reagent for detecting comprises a specific binding member selective for a particular biomarker.
320. A use according to statement 317 or 318, wherein the method of predicting a cancer-related outcome in a subject is a method according to any one of statements 101-196.
401. Use of one or more of: VDBP, THP, EpCam, EGFR, Fetuin-A, IL-6R beta, Axl, CFHR1, GDF-15, MSP, PON-1, insulin (C-peptide), and/or Apo A-II, as a biomarker for determining if a subject is susceptible to treatment with an agent capable of inhibiting or reversing EMT or a chemotherapeutic agent.
402. A use according to statement 401, wherein:
   (i) a higher activity, expression, or amount of one or more of said biomarkers in the subject, or sample derived from the subject, compared with a control profile for the respective biomarker is indicative of susceptibility to treatment with an agent capable of inhibiting or reversing EMT or a chemotherapeutic agent; or
   (ii) a lower activity, expression, or amount of one or more of said biomarkers in the subject, or sample derived from the subject, compared with a control profile for the respective biomarker is indicative of susceptibility to treatment with an agent capable of inhibiting or reversing EMT or a chemotherapeutic agent.
403. A use according to statement 401, wherein:
   (i) an increase in the activity, expression, or amount of one or more biomarker relative to a control profile for the respective biomarker after contacting the subject with a cancer therapeutic agent is indicative of increased susceptibility to treatment with an agent capable of inhibiting or reversing EMT or a chemotherapeutic agent; or
   (ii) a decrease in the activity, expression, or amount of one or more biomarker relative to a control profile for the respective biomarker after contacting the subject with a cancer therapeutic agent is indicative of increased susceptibility to treatment with an agent capable of inhibiting or reversing EMT or a chemotherapeutic agent.
404. A use according to any one of statements 401-403, wherein the use is as a biomarker in a method according to any one of statements 101-196
501. A method of treating a subject having, suspected of having, or diagnosed with acute myelocytic leukemia (AML) or myelodysplastic syndrome (MDS), the method comprising:
   identifying a subject susceptible to treatment using a method according to any one of statements 101-196; and
   administering to the subject a therapeutically effective amount of an agent capable of inhibiting or reversing EMT or a chemotherapeutic agent.
502. A method of treating a subject having, suspected of having, or diagnosed with acute myelocytic leukemia (AML) or myelodysplastic syndrome (MDS), the method comprising:
   (a) obtaining a sample from the subject;
   (b) determining if the subject is susceptible to treatment with an agent capable of inhibiting or reversing EMT or a chemotherapeutic agent using a method according to any one of statements 101-196;
   (c) administering to the subject a therapeutically effective amount of an agent capable of inhibiting or reversing EMT or a chemotherapeutic agent.
503. A method of treating a subject having, suspected of having, or diagnosed with acute myelocytic leukemia (AML) or myelodysplastic syndrome (MDS), the method comprising administering a therapeutically effective amount of an agent capable of inhibiting or reversing EMT or chemotherapeutic agent to a subject identified as susceptible to said treatment using a method according to any one of statements 101-196.
504. A method of treating a subject having, suspected of having, or diagnosed with acute myelocytic leukemia (AML) or myelodysplastic syndrome (MDS), the method comprising:
   administering to the subject a therapeutically effective amount of an agent capable of inhibiting or reversing EMT or a chemotherapeutic agent; and
   determining if the subject is susceptible to said treatment using a method according to any one of statements 156-196.
505. A method according to statement 504, wherein the method comprises administering to the subject one or more further therapeutically effective amounts of an agent capable of inhibiting or reversing EMT or a chemotherapeutic agent provided that the subject is identified as susceptible to said treatment.
506. An agent capable of inhibiting or reversing EMT or chemotherapeutic agent for use in a method of treating a subject having, suspected of having, or diagnosed with acute myelocytic leukemia (AML) or myelodysplastic syndrome (MDS),
   wherein the method is a method according to any one of statements 501-505.
507. Use of an agent capable of inhibiting or reversing EMT or chemotherapeutic agent for use in a method of manufacture of a medicament for use in a method of treating a subject having, suspected of having, or diagnosed with acute myelocytic leukemia (AML) or myelodysplastic syndrome (MDS),
   wherein the method of treatment is a method according to any one of statements 501-505.
508. A method, agent for use, or use according to any one of statements 501-507, wherein the agent capable of inhibiting or reversing EMT or chemotherapeutic agent comprises an Axl inhibitor, Akt3 inhibitor, Slfn11 inhibitor, or PHGDH inhibitor.
509. A method, agent for use, or use according to statement 508, wherein the agent capable of inhibiting or reversing EMT or chemotherapeutic agent comprises an Axl inhibitor.
510. A method, agent for use, or use according to statement 509, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
511. A method, agent for use, or use according to statement 510, wherein the Axl inhibitor is BGB324/R428/bemcentinib.
512. A method, agent for use, or use according to statement 510, wherein the Axl inhibitor is an anti-Axl antibody described in WO2015/193428, WO2015/193430, WO2016/097370, or WO2016/166296.
512. A method, agent for use, or use according to statement 508, wherein the agent capable of inhibiting or reversing EMT or chemotherapeutic agent comprises an Akt3 inhibitor.
513. A method, agent for use, or use according to statement 512, wherein the Akt3 inhibitor is an Akt3 inhibitor disclosed in WO2016/102672.
514. A method, agent for use, or use according to any one of statements 501-513, wherein the agent capable of inhibiting or reversing EMT or chemotherapeutic agent is administered as a single agent.
515. A method, agent for use, or use according to any one of statements 501-513, wherein the agent capable of inhibiting or reversing EMT or chemotherapeutic agent is administered in combination with a further cancer treatment.
516. A method, agent for use, or use according to statement 515, wherein the further cancer treatment is selected from the further cancer treatments listed in statement 130.
517. A method, agent for use, or use according to statement 515, wherein the further cancer treatment is a pyrimidine analogue, for example azacitidine, capecitabine, cytarabine, edatrexate, floxuridine, fluorouracil, gemcitabine, or troxacitabine, or a cytidine analogue, for example decitabine.
518. A method, agent for use, or use according to statement 517, wherein the further cancer treatment is cytarabine.
519. A method, agent for use, or use according to statement 517, wherein the further cancer treatment is decitabine.

Facet 4: Melanoma

Melanoma, also known as malignant melanoma, is a class of cancer that develops from melanocytes. These cancers typically occur in the skin, but can also occur in the mouth, intestines, or eye. Chemotherapeutic drug therapies for melanoma include, for example ipilimumab, pembrolizumab, and nivolumab. The overall success rate of such treatments in metastatic melanoma remains limited, and there remains a need for targeted therapies for this cancer type (Hsueh et al, 2016).

Accordingly, robust biomarkers for predicting cancer-related outcomes in a subject having melanoma, for example susceptibility to treatment with an agent capable of inhibiting or reversing EMT or a chemotherapeutic agent, would be useful in identifying subjects who are most likely to respond to or benefit from treatment with a particular agent/regimen.

A “responder” subject as described herein is a subject who is likely to, or who does, respond to treatment (or benefit from treatment) with an agent capable of inhibiting or reversing EMT or a chemotherapeutic agent, for example the small molecule Axl kinase inhibitor BGB324/R428/bemcentinib when administered either as a single agent or in combination with another cancer treatment.

Correspondingly, a “non-responder” subject as described herein is a subject who is not likely to, or who does not, respond to treatment (or benefit from treatment) with an agent capable of inhibiting or reversing EMT or a chemotherapeutic agent, for example the small molecule Axl kinase inhibitor BGB324/R428/bemcentinib when administered either as a single agent or in combination with another cancer treatment.

To “respond to treatment” or “benefit from treatment” as used herein means to experience an overall clinical benefit from the treatment. This overall clinical benefit can be any of: prolonged survival, partial or complete disease remission, slowing or absence of disease progression, tumour shrinkage (for example, a reduction in tumour volume of 5, 10, 20, 30, 40% or more), reduction in tumour burden (for example, a reduction in tumour burden of 5, 10, 20, 30, 40% or more), slowing or absence of tumour enlargement, slowing or absence of increase in tumour burden, improved quality of life, progression-free survival, overall survival, or any other positive patient outcome. Suitable methods for determining tumour volume/burden are well known to the skilled person, for example, using: computed tomography (CT), or magnetic resonance imaging (MRI) imaging technologies; X-ray imaging, for example, mammography; ultrasound imaging; nuclear imaging, for example positron emission tomography (PET), PET/CT scans, bone scans, gallium scans, or metaiodobenzylguanidine (MIBG) scans; bioluminescence imaging (BLI); fluorescence imaging (FLI); BD ToF (infrared-based 3D Time-of-Flight camera) imaging.

Biomarkers

The term “marker” or “biomarker” is used herein to refer to a gene or protein whose expression in a subject or sample derived from a subject is altered or modulated, for example, up or down regulated, in cancer. Where the biomarker is a protein, modulation or alteration of expression encompasses modulation through different post-translational modifications.

In accordance with any aspect of this facet of the present disclosure the biomarkers described herein may be defined as follows, wherein:
an Axl biomarker comprises an amino acid sequence having at least 70%, 80%, 90%, 95%, 99% or 100% sequence identity with the full-length of the amino acid sequence disclosed at UniProt accession no: P30530-1 or P30530-2 (entry version 196) or a fragment thereof;
an Interleukin-6 receptor (IL-6r) biomarker comprises an amino acid sequence having at least 70%, 80%, 90%, 95%, 99% or 100% sequence identity with the full-length of the amino acid sequence disclosed at UniProt accession no: P08887-1 or P08887-2 (entry version 210) or a fragment thereof;
a Chemokine CC-4 (HCC-4) biomarker comprises an amino acid sequence having at least 70%, 80%, 90%, 95%, 99% or 100% sequence identity with the full-length of the amino acid sequence disclosed at UniProt accession no: 015467 (entry version 146) or a fragment thereof;
a Tissue Inhibitor of Metalloproteinases 1 (TIMP-1) biomarker comprises an amino acid sequence having at least 70%, 80%, 90%, 95%, 99% or 100% sequence identity with the full-length of the amino acid sequence disclosed at UniProt accession no: P01033 (entry version 202) or a fragment thereof;
a Heparin-Binding EGF-Like Growth Factor (HB-EGF) biomarker comprises an amino acid sequence having at least 70%, 80%, 90%, 95%, 99% or 100% sequence identity with the full-length of the amino acid sequence disclosed at UniProt accession no: Q99075 (entry version 171) or a fragment thereof;
an Antileukoproteinase (ALP) biomarker comprises an amino acid sequence having at least 70%, 80%, 90%, 95%, 99% or 100% sequence identity with the full-length of the amino acid sequence disclosed at UniProt accession no: P03973 (entry version 184) or a fragment thereof;
a Latency-Associated Peptide of Transforming Growth Factor beta 1 (LAP TGF-b1) biomarker comprises an amino acid sequence having at least 70%, 80%, 90%, 95%, 99% or 100% sequence identity with the full-length of the amino acid sequence disclosed at UniProt accession no: P01137 (entry version 235) or a fragment thereof;
an Epidermal Growth Factor (EGF) biomarker comprises an amino acid sequence having at least 70%, 80%, 90%, 95%, 99% or 100% sequence identity with the full-length of the amino acid sequence disclosed at UniProt accession no: P01133-1, P01133-2, or P01133-3 (entry version 204) or a fragment thereof;
a Platelet-Derived Growth Factor BB (PDGF-BB) biomarker comprises an amino acid sequence having at least 70%, 80%, 90%, 95%, 99% or 100% sequence identity with the full-length of the amino acid sequence disclosed at UniProt accession no: P01127-1 or P01127-2 (entry version 217) or a fragment thereof;
a Osteoprotegerin (OPG) biomarker comprises an amino acid sequence having at least 70%, 80%, 90%, 95%, 99% or 100% sequence identity with the full-length of the amino acid sequence disclosed at UniProt accession no: 000300 (entry version 155) or a fragment thereof;
a Neutrophil Activating Peptide 2 (NAP-2) biomarker comprises an amino acid sequence having at least 70%, 80%, 90%, 95%, 99% or 100% sequence identity with the full-length of the amino acid sequence disclosed at UniProt accession no: P02775 (entry version 191) or a fragment thereof;
an ST2 biomarker comprises an amino acid sequence having at least 70%, 80%, 90%, 95%, 99% or 100% sequence identity with the full-length of the amino acid sequence disclosed at UniProt accession no: Q01638-1, Q01638-2, Q01638-3, or Q01638-4 (entry version 179) or a fragment thereof;
a Tumor Necrosis Factor Receptor I (TNF RI) biomarker comprises an amino acid sequence having at least 70%, 80%, 90%, 95%, 99% or 100% sequence identity with the full-length of the amino acid sequence disclosed at UniProt accession no: P19438-1, P19438-2, P19438-3, P19438-4, or P19438-5 (entry version 225) or a fragment thereof;
a Vascular Endothelial Growth Factor (VEGF) biomarker comprises an amino acid sequence having at least 70%, 80%, 90%, 95%, 99% or 100% sequence identity with the full-length of the amino acid sequence disclosed at UniProt accession no: P15692-1, P15692-2, P15692-3, P15692-4, P15692-5, P15692-6, P15692-7, P15692-8, P15692-9, P15692-10, P15692-11, P15692-12, P15692-13, P15692-14, P15692-15, P15692-16, P15692-17, or P15692-18 (entry version 230) or a fragment thereof;
a Growth-Regulated alpha protein (GRO-alpha) biomarker comprises an amino acid sequence having at least 70%, 80%, 90%, 95%, 99% or 100% sequence identity with the full-length of the amino acid sequence disclosed at UniProt accession no: P09341 (entry version 183) or a fragment thereof;
a Lactate Dehydrogenase (LDH) biomarker comprises an amino acid sequence having at least 70%, 80%, 90%, 95%, 99% or 100% sequence identity with the full-length of the amino acid sequence disclosed at UniProt accession no: P00338-1, P00338-2, P00338-3, P00338-4, or P00338-5 (entry version 224) or a fragment thereof; and/or
a Lactate Dehydrogenase (LDH) biomarker comprises an amino acid sequence having at least 70%, 80%, 90%, 95%, 99% or 100% sequence identity with the full-length of the amino acid sequence disclosed at UniProt accession no: P07195-1 (entry version 218) or a fragment thereof.

The biomarkers described herein may also be defined as a nucleic acid (either DNA or RNA) having at least 70%, 80%, 90%, 95%, 99% or 100% sequence identity with the full-length nucleic acid sequence encoding any of the above amino acid sequences.

Identity may be as defined using sequence comparisons made using FASTA and FASTP (see Pearson & Lipman, 1988. Methods in Enzymology 183: 63-98). Parameters are preferably set, using the default matrix, as follows: Gapopen (penalty for the first residue in a gap): −12 for proteins/-16 for DNA; Gapext (penalty for additional residues in a gap): −2 for proteins/-4 for DNA; KTUP word length: 2 for proteins/6 for DNA.

Pre-Treatment Predictive Biomarkers and Methods

By analysing patient data obtained from clinical trial BGBIL006 (NCT02872259), the authors have discovered that, prior to treatment of subjects having melanoma, certain of the biomarkers described herein are present in the serum of “responder” subjects at higher levels than in the serum of “non-responder” subjects—see Example 2 and Example 7. Similarly, prior to treatment of the subject certain of the biomarkers described herein are present in the serum of “responder” subjects at lower levels than in the serum of “non-responder” subjects. Thus, by assessing the activity, expression, or amount of one or more of the biomarkers described herein prior to treatment with an agent capable of inhibiting or reversing EMT or a chemotherapeutic agent it is possible to determine the likelihood of that subject being susceptible or responsive to treatment with said agent.

Accordingly, in a first aspect of this facet, the disclosure provides a method of predicting a cancer-related outcome in a subject having, suspected of having, or diagnosed with melanoma, the method comprising assessing the activity, expression, or amount of one or more of the biomarkers described herein in the subject, or in a sample derived from the subject.

In some embodiments the one or more biomarker is selected from the group consisting of: Axl, Interleukin-6 receptor (IL-6r), Chemokine CC-4 (HCC-4), Tissue Inhibitor of Metalloproteinases 1 (TIMP-1), Heparin-Binding EGF-Like Growth Factor (HB-EGF), Antileukoproteinase (ALP), Latency-Associated Peptide of Transforming Growth Factor beta 1 (LAP TGF-b1), Epidermal Growth Factor (EGF), Platelet-Derived Growth Factor BB (PDGF-BB), Osteoprotegerin (OPG), Neutrophil Activating Peptide 2 (NAP-2), ST2, Tumor Necrosis Factor Receptor I (TNF RI), Vascular Endothelial Growth Factor (VEGF), Growth-Regulated alpha protein (GRO-alpha), and/or Lactate Dehydrogenase (LDH).

In some embodiments the one or more biomarker is selected from the group consisting of: Axl, Interleukin-6 receptor (IL-6r), Chemokine CC-4 (HCC-4), Tissue Inhibitor of Metalloproteinases 1 (TIMP-1), Heparin-Binding EGF-Like Growth Factor (HB-EGF), Antileukoproteinase (ALP), Latency-Associated Peptide of Transforming Growth Factor beta 1 (LAP TGF-b1), Epidermal Growth Factor (EGF), Platelet-Derived Growth Factor BB (PDGF-BB), Osteoprotegerin (OPG), Neutrophil Activating Peptide 2 (NAP-2), ST2, Tumor Necrosis Factor Receptor I (TNF RI), Vascular Endothelial Growth Factor (VEGF), and/or Growth-Regulated alpha protein (GRO-alpha).

A “cancer-related outcome” as described herein is a clinical prediction or prognosis associated with cancer. In some embodiments the cancer-related outcome is susceptibility to treatment with an agent capable of inhibiting or reversing EMT or a chemotherapeutic agent.

In some embodiments the method comprises assessing the activity, expression, or amount of one or more of the biomarkers described herein in the subject, or in a sample derived from the subject, to obtain a sample profile of the one or more biomarker; and, making a prediction based on the sample profile of the one or more biomarker.

A “sample profile” as used herein is a profile of numerical values or numerical ranges corresponding to the activity, expression, or amount of respective biomarkers determined in the subject or sample derived from the subject.

In some embodiments of the method the prediction is made by comparing the sample profile to a control profile. A “control profile” as used herein is a profile of numerical values or numerical ranges corresponding to the activity, expression, or amount of the same biomarkers as for the sample profile, determined in a control subject or population of control subjects. Suitable control profiles are described in more detail below.

Prior to treatment of a subject, certain of the biomarkers described herein have been found to exhibit up-regulated expression in “responder” subjects—that is, certain of the biomarkers described herein have been found to be present in the serum of “responder” subjects at higher levels than in the serum of “non-responder” subjects. Similarly, prior to treatment of a subject, certain of the biomarkers described herein have been found to exhibit down-regulated expression in “responder” subjects—that is, certain of the biomarkers described herein have been found to be present in the serum of “responder” subjects at lower levels than in the serum of “non-responder” subjects.

This difference in expression level between “responder” and “non-responder” subjects can also be expressed in the opposite way. That is to say, prior to treatment of a subject, certain of the biomarkers described herein have been found to exhibit down-regulated expression in “non-responder” subjects—that is, certain of the biomarkers described herein have been found to be present in the serum of “non-responder” subjects at lower levels than in the serum of “responder” subjects. Similarly, prior to treatment of a subject, certain of the biomarkers described herein have been found to exhibit up-regulated expression in “non-responder” subjects—that is, certain of the biomarkers described herein have been found to be present in the serum of “non-responder” subjects at higher levels than in the serum of “responder” subjects.

The discovery of these differential patterns of expression mean that, by assessing the activity, expression, or amount of one or more of the biomarkers described herein prior to treatment of a subject with an agent, it is possible to determine the likelihood of that subject being susceptible or responsive to treatment with said agent. Accordingly, in particularly preferred embodiments the sample profile is obtained before the subject is contacted with or administered the agent capable of inhibiting or reversing EMT or a chemotherapeutic agent.

A subject who is “susceptible to treatment” with an agent capable of inhibiting or reversing EMT or a chemotherapeutic agent is one who is likely to, or who does, respond to treatment (or benefit from treatment) with an agent capable of inhibiting or reversing EMT or a chemotherapeutic agent. That is, a subject who is “susceptible to treatment” with an agent is a “responder” subject for that agent, as defined above. A prediction that is “indicative of susceptibility” is one which suggests or points towards the likelihood that a subject will respond to or benefit from treatment.

In some embodiments the predictive methods of the disclosure comprise assessing the activity, expression, or amount of one or more of the biomarkers described herein in the subject, or in a sample derived from the subject, to obtain a sample profile of the one or more biomarker; and, making a prediction based on the sample profile of the one or more biomarker. In some embodiments of the method the prediction is made by comparing the sample profile to a control profile, wherein the control profile is a profile of numerical values or numerical ranges corresponding to the activity, expression, or amount of the same biomarkers as for the sample profile, determined in a control subject or population of control subjects.

In some embodiments the control profile may be obtained from a population of control subjects having melanoma. In some embodiments the control profile may be obtained from a population of control subjects not having melanoma. In other embodiments the control profile may be obtained from a control subject or population of control subjects having melanoma and previously found to lack susceptibility to treatment with an agent capable of inhibiting or reversing EMT or a chemotherapeutic agent (that is, non-responder subjects).

In some embodiments the control profile may a predetermined profile of biomarker expression, activity, or amount values, for example a profile of “mean” values, “threshold” values, or “standard ranges” of values. This predetermined profile of biomarker expression, activity, or amount values can be obtained, for example, from a control subject or population of control subjects having melanoma, a control subject or population of control subjects not having melanoma, or a control subject or population of control subjects having melanoma and previously found to lack susceptibility to treatment with an agent capable of inhibiting or reversing EMT or a chemotherapeutic agent (that is, non-responder subjects).

In some other embodiments the control profile may be obtained from a control subject or population of control subjects having melanoma and previously found to be susceptible to treatment with an agent capable of inhibiting or reversing EMT or a chemotherapeutic agent (that is, responder subjects). In some embodiments the control profile may a predetermined profile of biomarker expression, activity, or amount values, for example a profile of “threshold”, values or “standard ranges” of values. This predetermined profile of biomarker expression, activity, or amount values can be obtained, for example, from a control subject or population of control subjects having melanoma and previously found to be susceptible to treatment with an agent capable of inhibiting or reversing EMT or a chemotherapeutic agent (that is, responder subjects).

In some other embodiments the control profile may be obtained from a control sample having a known amount (for example a “threshold” amount) of the same biomarkers as will be determined in the sample profile. This threshold amount can be obtained, for example, from non-responder or responder subjects as outlined above.

The skilled person is readily able to determine suitable “mean”, “threshold”, or “standard range” values for a particular biomarker.

For example, a “mean” value may be determined by: assessing the activity, expression, or amount of a particular biomarker in a population of subjects having (or not having) melanoma; and, averaging the biomarker activity, expression, or amount determined to determine a “mean” value.

For example, a “mean” value may be determined by: assessing the activity, expression, or amount of a particular biomarker in a population of subjects; determining which subjects in the population are susceptible to treatment with an agent capable of inhibiting or reversing EMT or a chemotherapeutic agent; and, averaging the biomarker activity, expression, or amount determined in subjects found to be susceptible to treatment with an agent capable of inhibiting or reversing EMT or a chemotherapeutic agent (that is, responder subjects) to determine a “mean” value.

For example, a “threshold” value may be determined by: assessing the activity, expression, or amount of a particular biomarker in a population of subjects; determining which subjects in the population are susceptible to treatment with an agent capable of inhibiting or reversing EMT or a chemotherapeutic agent; and, selecting as appropriate the highest or lowest biomarker activity, expression, or amount determined in a subject found to be susceptible to treatment with an agent capable of inhibiting or reversing EMT or a chemotherapeutic agent (that is, a responder subject) as the “threshold value”.

For example, a “standard range” of values may be determined by: assessing the activity, expression, or amount of a particular biomarker in a population of subjects having (or not having) melanoma; and, determining a “standard range” of values based on this assessment.

For example, a “standard range” of values may be determined by: assessing the activity, expression, or amount of a particular biomarker in a population of subjects; determining which subjects in the population are susceptible to treatment with an agent capable of inhibiting or reversing EMT or a chemotherapeutic agent; and, determining a “standard range” of values for subjects found to be susceptible to treatment with an agent capable of inhibiting or reversing EMT or a chemotherapeutic agent (that is, a responder subject) based on this assessment.

Where the control profile is a predetermined profile of biomarker expression, activity, or amount values, this may be provided as a “look-up table” or data record. Predetermined control profiles of this type may be a profile of numerical values or numerical ranges corresponding to a measure of central tendency (such as an average, median or mean) amount of the respective biomarkers determined in a plurality of control subjects (for example non-responder or responder subjects as outlined above). The skilled person is readily able to determine both a measure of central tendency and a measure of variability within a population using standard protein quantification techniques and suitable statistical tools.

Accordingly, in embodiments in which the control profile is: obtained from a population of control subjects having melanoma; obtained from a population of control subjects not having melanoma; obtained from a control subject or population of control subjects having melanoma and previously found to lack susceptibility to treatment with an agent capable of inhibiting or reversing EMT or a chemotherapeutic agent (that is non-responder subjects); a predetermined profile of biomarker expression, activity, or amount values (for example a profile of “average, median, or mean” values or “standard ranges” of values) obtained from non-responder subjects; obtained from a control sample having a known “average, median, or mean” value of biomarkers indicative of non-responder subjects; a predetermined profile of biomarker expression, activity, or amount “threshold” values obtained from responder subjects; or, obtained from a control sample having a known “threshold” value of biomarkers indicative of responder subjects; then, a higher activity, expression, or amount of one or more of the biomarkers described herein in a sample profile as compared with the control profile may be indicative of susceptibility to treatment with an agent capable of inhibiting or reversing EMT or a chemotherapeutic agent. Similarly, a lower activity, expression, or amount of one or more of the biomarkers described herein in the sample profile as compared with a control profile may be indicative of susceptibility to treatment with an agent capable of inhibiting or reversing EMT or a chemotherapeutic agent.

In some such embodiments, the method may comprise assessing the activity, expression, or amount of one or more of biomarker selected from: TIMP-1, HB-EGF, ALP, LAP TGF-b1, EGF, PDGF-BB, OPG, NAP-2, ST2, TNF RI, VEGF, GRO-alpha, and/or LDH; wherein a higher activity, expression, or amount of one or more of said biomarkers in the sample profile as compared with the control profile is indicative of susceptibility to treatment with an agent capable of inhibiting or reversing EMT or a chemotherapeutic agent.

In some such embodiments, the method may comprise assessing the activity, expression, or amount of one or more of biomarker selected from: Axl, IL-6r, and/or HCC-4; wherein a lower activity, expression, or amount of one or more of said biomarkers in the sample profile as compared with the control profile is indicative of susceptibility to treatment with an agent capable of inhibiting or reversing EMT or a chemotherapeutic agent.

In some such embodiments: a lower activity, expression, or amount of Axl; and/or a lower activity, expression, or amount of IL-6r; and/or a lower activity, expression, or amount of HCC-4; and/or a higher activity, expression, or amount of TIMP-1; and/or a higher activity, expression, or amount of HB-EGF; and/or a higher activity, expression, or amount of ALP; and/or a higher activity, expression, or amount of LAP TGF-b1; and/or a higher activity, expression, or amount of EGF; and/or a higher activity, expression, or amount of PDGF-BB; and/or a higher activity, expression, or amount of OPG; and/or a higher activity, expression, or amount of NAP-2; and/or a higher activity, expression, or amount of ST2; and/or a higher activity, expression, or amount of TNF RI; and/or a higher activity, expression, or amount of VEGF; and/or a higher activity, expression, or amount of GRO-alpha; and/or a higher activity, expression, or amount of LDH in the sample profile as compared with the control profile is indicative of susceptibility to treatment with an agent capable of inhibiting or reversing EMT or a chemotherapeutic agent.

In embodiments in which the control profile is: obtained from a control subject or population of control subjects having melanoma and previously found to be susceptible to treatment with an agent capable of inhibiting or reversing EMT or a chemotherapeutic agent (that is, responder subjects); then, if the expression, activity, or amount values in the sample profile are above a “threshold” value typical of a responder subject (that is, a subject having melanoma and previously found to be susceptible to treatment with an agent capable of inhibiting or reversing EMT or a chemotherapeutic agent) this is indicative of susceptibility to treatment with an agent capable of inhibiting or reversing EMT or a chemotherapeutic agent. Similarly, if the expression, activity, or amount values in the sample profile are below the “threshold” value typical of a responder subject (that is, a subject having melanoma and previously found to be susceptible to treatment with an agent capable of inhibiting or reversing EMT or a chemotherapeutic agent) this is indicative of a lack of susceptibility to treatment with an agent capable of inhibiting or reversing EMT or a chemotherapeutic agent.

In embodiments in which the control profile is: a predetermined profile of “standard ranges” of biomarker expression, activity, or amount values obtained from responder subjects; then, if the expression, activity, or amount values in the sample profile are within the “standard range” of values typical of a responder subject (that is, a subject having melanoma and previously found to be susceptible to treatment with an agent capable of inhibiting or reversing EMT or a chemotherapeutic agent) this is indicative of susceptibility to treatment with an agent capable of inhibiting or reversing EMT or a chemotherapeutic agent. Similarly, if the expression, activity, or amount values in the sample profile are outside the “standard range” of values typical of a responder subject (that is, a subject having melanoma and previously found to be susceptible to treatment with an agent capable of inhibiting or reversing EMT or a chemotherapeutic agent) this is indicative of a lack of susceptibility to treatment with an agent capable of inhibiting or reversing EMT or a chemotherapeutic agent.

In embodiments in which the control profile is: a predetermined profile of “standard ranges” of biomarker expression, activity, or amount values obtained from non-responder subjects; then, if the expression, activity, or amount values in the sample profile are within the “standard range” of values typical of a non-responder subject (that is, a subject having melanoma and previously found to lack susceptibility to treatment with an agent capable of inhibiting or reversing EMT or a chemotherapeutic agent) this is indicative of a lack of susceptibility to treatment with an agent capable of inhibiting or reversing EMT or a chemotherapeutic agent. Similarly, if the expression, activity, or amount values in the sample profile are outside the “standard range” of values typical of a non-responder subject (that is, a subject having melanoma and previously found to lack susceptibility to treatment with an agent capable of inhibiting or reversing EMT or a chemotherapeutic agent) this may be indicative of susceptibility to treatment with an agent capable of inhibiting or reversing EMT or a chemotherapeutic agent.

In embodiments in which the control profile is: obtained from a population of control subjects not having melanoma; then, if the expression, activity, or amount values in the sample profile are outside a “standard range” of values typical of subjects not having melanoma, this is indicative of susceptibility to treatment with an agent capable of inhibiting or reversing EMT or a chemotherapeutic agent. Similarly, if the expression, activity, or amount values in the sample profile are within a “standard range” of values typical of subjects not having melanoma, this is indicative of a lack of susceptibility to treatment with an agent capable of inhibiting or reversing EMT or a chemotherapeutic agent. In such cases, an expression, activity or amount value may be outside the “standard range” of values when it is either above the upper limit of normal or below the lower limit of normal for a given biomarker. The upper limit of normal and lower limit of normal are the limits of the normal distribution of a given biomarker in a population of control subjects not having melanoma.

In embodiments in which the one or more biomarker comprises LDH, an expression or amount of LDH higher than 287 units per litre (U/L) may be indicative of susceptibility to treatment with an agent capable of inhibiting or reversing EMT or a chemotherapeutic agent.

The skilled person is readily able to determine whether the sample profile is indicative of susceptibility or lack of susceptibility to treatment with an agent capable of inhibiting or reversing EMT or a chemotherapeutic agent using suitable statistical tools and comparison with the appropriate control.

In some embodiments the sample profile may be compared with two or more control profiles as described above.

As used herein, the term “one or more” means 1, 2, 3, 4, 5, 6, 7, 8, 9, 10, 11, 12, 13, 14, 15, 16, 17, 18, 19, or 20 of the biomarkers described herein. In some embodiments the one or more biomarker is Axl. In some embodiments the one or more biomarker is IL-6r. In some embodiments the one or more biomarker is HCC-4. In some embodiments the one or more biomarker is TIMP-1. In some embodiments the one or more biomarker is HB-EGF. In some embodiments the one or more biomarker is ALP. In some embodiments the one or more biomarker is LAP TGF-b1. In some embodiments the one or more biomarker is EGF. In some embodiments the one or more biomarker is PDGF-BB. In some embodiments the one or more biomarker is OPG. In some embodiments the one or more biomarker is NAP-2. In some embodiments the one or more biomarker is ST2. In some embodiments the one or more biomarker is TNF RI. In some embodiments the one or more biomarker is VEGF. In some embodiments the one or more biomarker is GRO-alpha. In some embodiments the one or more biomarker is LDH. In some preferred embodiments the one or more biomarker is Axl. In some preferred embodiments the one or more biomarker is LDH. In other preferred embodiments the one or more biomarker is Axl and LDH.

In some embodiments the method may comprise assessing the activity, expression, or amount of at least 2, 3, 4, 5, 6, 7, 8, 9, 10, 11, 12, 13, 14, 15, 20 of said biomarkers in the subject, or in a sample derived from the subject. In some embodiments the method may comprise assessing the activity, expression, or amount of Axl and at least 1, 2, 3, 4, 5, 6, 7, 8, 9, 10, 11, 12, 13, 14, 15, 20 further biomarkers described herein in the subject, or in a sample derived from the subject. In some embodiments the method may comprise assessing the activity, expression, or amount of LDH and at least 1, 2, 3, 4, 5, 6, 7, 8, 9, 10, 11, 12, 13, 14, 15, 20 further biomarkers described herein in the subject, or in a sample derived from the subject. In some embodiments the method may comprise assessing the activity, expression, or amount of Axl, LDH, and at least 1, 2, 3, 4, 5, 6, 7, 8, 9, 10, 11, 12, 13, 14, 15, 20 further biomarkers described herein in the subject, or in a sample derived from the subject.

Subject

A “subject” as referred to herein is any species of animal classified as a mammal, and includes but is not restricted to, domestic and farm animals, primates and humans. In some preferred embodiments the subject is a human of any sex or race. In some embodiments, the human is an adult human.

In some embodiments the predictive methods described herein are performed before the subject receives a treatment or course of treatment for melanoma. In other embodiments the predictive method is performed during or after a treatment or course of treatment for melanoma. In some embodiments the treatment or course of treatment for melanoma is an agent capable of inhibiting or reversing EMT. In other embodiments the treatment or course of treatment for melanoma is a chemotherapeutic treatment that is not an agent capable of inhibiting or reversing EMT.

In some embodiments of the predictive methods described herein the subject has previously been treated with an agent capable of inhibiting or reversing EMT or a chemotherapeutic agent. In some embodiments the subject has previously been treated with an agent selected from: a PHGDH inhibitor, a Slfn11 inhibitor, an Axl inhibitor, or an Akt3 inhibitor.

In some embodiments of the predictive methods described herein the subject has previously been treated with an agent that is different to the agent capable of inhibiting or reversing EMT or chemotherapeutic agent for which the subject's response is being predicted. That is, the subject has previously been treated with an agent that is not the agent which the predictive method is determining the subject's susceptibility to. Accordingly, in some embodiments, the subject has not previously been treated with an agent capable of inhibiting or reversing EMT or a chemotherapeutic agent. In some preferred embodiments, the subject has not previously been treated with an Axl inhibitor. In other preferred embodiments, the subject has not previously been treated with an Akt3 inhibitor.

Post-Treatment Changes in Biomarkers

In some embodiments of this aspect of the disclosure, the predictive method further comprises a subsequent step of assessing the activity, expression, or amount of one or more biomarker in the subject, or in a sample derived from the subject, to obtain a second sample profile of one or more of the biomarkers described herein after the subject has received a treatment or course of treatment an agent capable of inhibiting or reversing EMT or a chemotherapeutic agent.

That is, in some embodiments, the predictive method comprises:
(a) determining if a subject is susceptible to treatment with an agent capable of inhibiting or reversing EMT or a chemotherapeutic agent by assessing the activity, expression, or amount of one or more biomarker in the subject, as outlined above;
(b) administering to the subject a therapeutically effective amount of an agent capable of inhibiting or reversing EMT or a chemotherapeutic agent; and
(c) subsequently assessing the activity, expression, or amount of one or more biomarker in the subject, or in a sample derived from the subject.

In some embodiments, the predictive method comprises:
(a) determining if a subject is susceptible to treatment with an agent capable of inhibiting or reversing EMT or a chemotherapeutic agent by assessing the activity, expression, or amount of one or more biomarker in the subject, or in a sample derived from the subject, wherein the one or more biomarker is selected from the group consisting of: Axl, IL-6r, HCC-4, TIMP-1, HB-EGF, ALP, LAP TGF-b1, EGF, PDGF-BB, OPG, NAP-2, ST2, TNF RI, VEGF, GRO-alpha, and/or LDH;
(b) administering to the subject a therapeutically effective amount of an agent capable of inhibiting or reversing EMT or a chemotherapeutic agent; and
(c) subsequently assessing the activity, expression, or amount of one or more biomarker in the subject, or in a sample derived from the subject.

In some embodiments, the predictive method comprises:
(a) determining if a subject is susceptible to treatment with an agent capable of inhibiting or reversing EMT or a chemotherapeutic agent by assessing the activity, expression, or amount of one or more biomarker in the subject, or in a sample derived from the subject, wherein the one or more biomarker is selected from the group consisting of: Axl, IL-6r, HCC-4, TIMP-1, HB-EGF, ALP, LAP TGF-b1, EGF, PDGF-BB, OPG, NAP-2, ST2, TNF RI, VEGF, and/or GRO-alpha;
(b) administering to the subject a therapeutically effective amount of an agent capable of inhibiting or reversing EMT or a chemotherapeutic agent; and
(c) subsequently assessing the activity, expression, or amount of one or more biomarker in the subject, or in a sample derived from the subject.

In some preferred embodiments the methods comprise subsequently assessing the activity, expression, or amount of Axl in the subject, or in a sample derived from the subject. In some embodiments an increase in the activity, expression, or amount of Axl in the second sample profile as compared with a control profile after contacting the subject with a cancer therapeutic agent is indicative of higher susceptibility to treatment with an agent capable of inhibiting or reversing EMT or a chemotherapeutic agent. In some preferred embodiments the control profile is obtained from the same subject prior to treatment with an agent capable of inhibiting or reversing EMT or a chemotherapeutic agent.

Post-Treatment Predictive Biomarkers and Methods

By analysing patient data obtained from clinical trial BGBIL006 (NCT02872259), the authors have discovered that, following treatment of subjects having melanoma with an agent capable of inhibiting or reversing EMT or a chemotherapeutic agent, the levels of certain of the biomarkers described herein increases in the serum of “responder” subjects but not in the serum of “non-responder” subjects. Similarly, following treatment with an agent capable of inhibiting or reversing EMT or a chemotherapeutic agent, the levels of certain of the biomarkers described herein decreases in the serum of “responder” subjects but not in the serum of “non-responder” subjects. Thus, by assessing the activity, expression, or amount of one or more of the biomarkers described herein prior to and following treatment with an agent capable of inhibiting or reversing EMT or a chemotherapeutic agent it is possible to determine or confirm that the subject is susceptible or responsive to treatment with said agent.

Accordingly, in an second aspect of this facet the disclosure provides a method of predicting a cancer-related outcome in a subject having, suspected of having, or diagnosed with melanoma, the method comprising assessing the activity, expression, or amount of one or more of the biomarkers described herein in a subject, or in a sample derived from the subject after the subject has been contacted with an agent capable of inhibiting or reversing EMT or a chemotherapeutic agent. In some embodiments the cancer-related outcome is susceptibility to treatment with an agent capable of inhibiting or reversing EMT or a chemotherapeutic agent.

In some embodiments the method comprises assessing the activity, expression, or amount of one or more of the biomarkers described herein in the subject, or in a sample derived from the subject, to obtain a sample profile of the one or more biomarker; and, making a prediction based on the sample profile of the one or more biomarker. In some embodiments of the method the prediction is made by comparing the sample profile to a control profile. In preferred embodiments the sample profile is obtained after contacting the subject with an agent capable of inhibiting or reversing EMT or a chemotherapeutic agent.

A “control profile” as used herein is a profile of numerical values or numerical ranges corresponding to the activity, expression, or amount of the same biomarkers as for the sample profile, determined in a control subject or population of control subjects.

In preferred embodiments of this aspect of the disclosure the control profile is obtained from the same subject prior to treatment with an agent capable of inhibiting or reversing EMT or a chemotherapeutic agent. In some embodiments the control profile is obtained from a population of control subjects having melanoma. In other embodiments, the control profile is a predetermined profile of biomarker expression, activity, or amount values, for example a profile of “threshold” values or “standard ranges” of values. Accordingly, in embodiments in which the control profile is obtained from the same subject prior to treatment with an agent capable of inhibiting or reversing EMT or a chemotherapeutic agent, an increase in the activity, expression, or amount of one or more biomarker in the sample profile as compared with a control profile after contacting the subject with an agent capable of inhibiting or reversing EMT or a cancer therapeutic agent may be indicative of susceptibility to treatment with an agent capable of inhibiting or reversing EMT or a chemotherapeutic agent. In such embodiments, a decrease in the activity, expression, or amount of one or more biomarker in the sample profile as compared with a control profile after contacting the subject with a cancer therapeutic agent may be indicative of higher susceptibility to treatment with an agent capable of inhibiting or reversing EMT or a chemotherapeutic agent.

In some such embodiments, an increase in the activity, expression, or amount of one or more biomarker in the sample profile as compared with a control profile after contacting the subject with a cancer therapeutic agent may be indicative of lower susceptibility to treatment with an agent capable of inhibiting or reversing EMT or a chemotherapeutic agent. In some such embodiments, a decrease in the activity, expression, or amount of one or more biomarker in the sample profile as compared with a control profile after contacting the subject with a cancer therapeutic agent may be indicative of lower susceptibility to treatment with an agent capable of inhibiting or reversing EMT or a chemotherapeutic agent.

In some preferred embodiments the one or more biomarker includes at least Axl. In some embodiments the one or more biomarker is Axl. In some embodiments an increase in the activity, expression, or amount of Axl in the sample profile as compared with a control profile after contacting the subject with a cancer therapeutic agent is indicative of higher susceptibility to treatment with an agent capable of inhibiting or reversing EMT or a chemotherapeutic agent.

Subject

A “subject” as referred to herein is any species of animal classified as a mammal, and includes but is not restricted to, domestic and farm animals, primates and humans. In some preferred embodiments the subject is a human of any sex or race. In some embodiments, the human is an adult human.

In some embodiments of this aspect of the disclosure, the subject was selected for treatment with the agent capable of inhibiting or reversing EMT or a chemotherapeutic agent, using a predictive method according to the first aspect of this facet of the disclosure outlined above.

Agents Capable of Inhibiting or Reversing EMT

The present disclosure provides methods for predicting a cancer-related outcome in a subject having, suspected of having, or diagnosed with melanoma. A “cancer-related outcome” is a clinical prediction or prognosis associated with cancer.

The agents capable of inhibiting or reversing EMT for use in this melenoma facet of the disclosure are defined as described above in the AML facet (see identically titled section above on page 14, line 19 to page 17, line 2), except with preferred embodiments as set out below.

In some preferred embodiments the further cancer treatment is a PD-1 inhibitor or PD-L1 inhibitor. In some preferred embodiments the further cancer treatment is a monoclonal antibody such as pembrolizumab, alemtuzumab, bevacizumab, cetuximab, ibritumomab tiuxetan, or rituximab, or is a B-Raf inhibitor such as debrafenib, a MEK inhibitor such as trametinib or a combination therapy of these agents. In some particularly preferred embodiments the further cancer treatment is pembrolizumab. In other particularly preferred embodiments the further cancer treatment is a combination therapy of debrafenib and trametinib.

Assessing Biomarker Activity, Expression, Amount

In some embodiments of the predictive methods described herein, assessing the activity, expression, or amount of one or more biomarker in a subject, or in a sample derived from a subject, comprises: contacting a sample, or an extract from a sample, with at least 1, 2, 3, 4, 5, 6, 7, 8, 9, 10 or more reagents for detecting the activity, expression, or amount of one or more biomarkers, each of which is selective for a particular biomarker; and, detecting and/or quantifying said reagents for detecting.

In some embodiments comprising assessing the activity of LDH, assessing the activity of LDH comprises determining the enzymatic activity of LDH in the subject or sample derived from the subject. Preferably the enzymatic activity is assessed by determining the level or amount of a substrate or product of the enzymatic reaction catalysed by LDH in the subject or sample derived from the subject. In particularly preferred embodiments the enzymatic activity is assessed by determining the level or amount of reduced nicotinamide adenine dinucleotide (NADH) in the subject or sample derived from the subject. In some embodiments this may be determined using a colorimetric assay, for example, the colorimetric assay available from Sigma-Aldrich with catalogue number MAK066. Other suitable assays for assessing activity of LDH are well known to the skilled person. In particularly preferred embodiments the activity of LDH is assessed by determining the enzymatic activity of LDH in blood or serum.

In some preferred embodiments, assessing the expression or amount of the one or more biomarker comprises determining the level of the one or more biomarker protein or mRNA in the subject or sample derived from the subject. Preferably the expression or amount of the one or more biomarker is assessed by determining the level of protein expression. In particularly preferred embodiments the expression or amount of the one or more biomarker is assessed by determining the level of protein expression in serum.

The term “expression” refers to the transcription of a gene's DNA template to produce the corresponding mRNA and translation of this mRNA to produce the corresponding gene product (i.e., a peptide, polypeptide, or protein) as well as the “expression” of a protein in one or more forms that may have been modified post translation.

In some embodiments determining the level of the one or more biomarker protein or mRNA comprises: contacting a sample, or an extract from a sample, with at least 1, 2, 3, 4, 5, 6, 7, 8, 9, 10 or more specific binding members, each of which selectively binds to a respective biomarker protein or mRNA; and, detecting and/or quantifying formation of complex formed by said specific binding member and said biomarker protein or mRNA. In some embodiments the specific binding member may comprise an antibody molecule or a binding fragment thereof.

A range of suitable techniques for determining protein and mRNA expression levels are well known in the art, for example microarray analysis, Western blotting, and PCR techniques such as QPCR. In some embodiments, determining the expression or amount of the one or more biomarker protein or mRNA may comprise use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.

In some embodiments, the expression or amount of the one or more biomarker is determined as an absolute amount of the respective biomarker protein or mRNA in the sample. In such cases, the respective biomarker protein or mRNA may be quantified in the sample and the amount expressed as a measure of the sample, for example the volume, mass, or total protein content of the sample.

In some embodiments, the expression or amount of the one or more biomarker is determined as a relative amount of the respective biomarker protein or mRNA in the sample, wherein the relative amount is determined relative to a reference protein or mRNA in the sample. Determining the relative amount of biomarker protein or mRNA in this way allows for normalisation to, e.g., account for differences in total protein concentration and to remove bias from sample to sample.

Preferably the reference protein or mRNA is one whose expression or amount does not vary significantly between subjects having cancer and subjects not having cancer, in particular melanoma. That is, the reference protein or mRNA is preferably one whose expression or amount is not altered by melanoma. Accordingly, the reference protein or mRNA may comprise one or more protein or mRNA which is not one of the biomarkers described herein. When expressing the expression or amount of the one or more biomarker as a relative amount, the absolute amount of the respective biomarker protein or mRNA in the sample may be divided by the absolute amount of the reference protein or mRNA in the sample.

Assessing Biomarker Expression at the RNA Level

Gene expression can be detected at the RNA level. RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland). Typical assay formats utilising ribonucleic acid hybridisation include nuclear run-on assays, RT-PCR, RNase protection assays (Melton et al., Nuc. Acids Res. 12:7035), Northern blotting and In Situ hybridization. Gene expression can also be detected by microarray analysis as described below.

Assessing Biomarker Expression at the Protein Level

Preferably biomarker level is assessed by measuring protein expression. Altered gene or protein expression may also be detected by measuring the polypeptides encoded by the gene. This may be achieved by using molecules which bind to the polypeptides encoded by the biomarker gene. Suitable molecules/agents which bind either directly or indirectly to the polypeptides in order to detect the presence of the protein include naturally occurring molecules such as peptides and proteins, for example antibodies, or they may be synthetic molecules.

Antibodies may be derived from commercial sources or through techniques which are familiar to those skilled in the art. In one embodiment, and where altered expression manifests itself through the expression of alteration of post translationally-modified forms of a protein biomarker, antibodies specific for those different forms may be used. For the purposes of this disclosure, the term “antibody”, unless specified to the contrary, includes whole antibodies, or fragments of whole antibodies which retain their binding activity for a target antigen. Such fragments include Fv, F(ab′) and F(ab′)2 fragments, as well as single chain antibodies (scFv). Furthermore, the antibodies and fragments thereof may be humanised antibodies, for example as described in EP239400A. For example: monoclonal and polyclonal antibodies, recombinant antibodies, proteolytic and recombinant fragments of antibodies (Fab, Fv, scFv, diabodies), single-domain antibodies (VHH, sdAb, nanobodies, IgNAR, VNAR), and proteins unrelated to antibodies, which have been engineered to have antibody-like specific binding. Antibodies may be bound to a solid support and/or packaged into kits in a suitable container along with suitable reagents, controls, instructions and the like.

Arrays

Array technology and the various techniques and applications associated with it are well known in the art. Array technology overcomes the disadvantages with traditional methods in molecular biology, which generally work on a “one gene in one experiment” basis, resulting in low throughput and the inability to appreciate the “whole picture” of gene function. In the context of the products and methods of the present disclosure, array technology can be used, for example, in the analysis of the expression of biomarker proteins or mRNA.

In general, any library or group of samples may be arranged in an orderly manner into an array, by spatially separating the members of the library or group. Examples of suitable libraries for arraying include nucleic acid libraries (including DNA, cDNA, oligonucleotide, etc. libraries), peptide, polypeptide and protein libraries, as well as libraries comprising any molecules, such as ligand libraries, among others. Accordingly, where reference is made to a “library” in this document, unless the context dictates otherwise, such reference should be taken to include reference to a library in the form of an array.

Proteins, polypeptides, etc., may also be immobilised in arrays. For example, antibodies have been used in microarray analysis of the proteome using protein chips (Borrebaeck C A, 2000, Immunol Today 21(8):379-82). Polypeptide arrays are reviewed in, for example, MacBeath and Schreiber, 2000, Science, 289(5485):1760-1763.

Sample

Suitable samples include, but are not limited to, tissue samples such as tissue biopsy, blood, urine, buccal scrapes etc., as well as serum, plasma, or tissue culture supernatant samples. In preferred embodiments the expression or amount of the one or more biomarker is assessed by determining the level of protein expression in the sample. In some preferred embodiments the sample is a blood, serum, or plasma sample. In some particularly preferred embodiments the sample is a serum sample.

When assessing the expression or amount of the one or more biomarker proteins or mRNA in serum or plasma samples derived from a subject, samples are removed and subjected to analytical techniques such as flow cytometry, mass cytometry (CyTOF), ELISA, PET, and SELDI-TOF MS. In some embodiments, the method may comprise extracting RNA from said sample and detecting gene expression by QPCR. In some other embodiments, gene expression may be detected by detecting protein products by, for example, Western Blot.

In some embodiments, the level of expression of the one or more biomarker is assessed by determining the copy number of the gene encoding the one or more biomarker. Copy number (i.e. gene duplication events) may be determined using standard techniques known in the art, e.g. using a DNA chip as described in Jiang et al. (Jiang Q, Ho Y Y, Hao L, Nichols Berrios C, Chakravarti A. Copy number variants in candidate genes are genetic modifiers of Hirschsprung disease. PLoS One. 2011; 6(6)).

Is some preferred embodiments of the predictive methods described herein, the method is performed in vitro or ex vivo.

Methods of Selecting a Subject for Treatment

A third aspect of this facet of the present disclosure relates to methods of selecting subjects having, suspected of having, or diagnosed with melanoma for treatment with an agent capable of inhibiting or reversing EMT or a chemotherapeutic agent. In some embodiments the method comprises: identifying subjects susceptible to treatment with an agent capable of inhibiting or reversing EMT or a chemotherapeutic agent using a predictive method defined in accordance with the above described first and second aspects of this facet of the disclosure; and, selecting thus identified subjects for treatment.

In some embodiments this aspect relates to methods of selecting subjects having, suspected of having, or diagnosed with melanoma for continued treatment with an agent capable of inhibiting or reversing EMT or a chemotherapeutic agent. In some embodiments the method comprises: identifying subjects susceptible to treatment with an agent capable of inhibiting or reversing EMT or a chemotherapeutic agent using a predictive method defined in accordance with the above described second aspect of the disclosure; and, selecting thus identified subjects for treatment.

In some embodiments of this aspect of the disclosure, the treatment comprises administering to the subject a therapeutically effective amount of an agent capable of inhibiting or reversing EMT or a chemotherapeutic agent as described herein. Accordingly, in some embodiments the treatment comprises an Axl inhibitor, Akt3 inhibitor, Slfn11 inhibitor, or PHGDH inhibitor. In some preferred embodiments the treatment comprises an Axl inhibitor, for example, BGB324/R428/bemcentinib, cabozantinib, TP-0903, foretinib, merestinib, bosutinib, gilteritinib, crizotinib, amuvatinib, sunitinib, sitravatinib, LDC1267, or UNC2025. Other Axl inhibitors include the anti-Axl antibodies described in WO2015/193428, WO2015/193430, WO2016/097370, and WO2016/166296. In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In some other preferred embodiments, the treatment comprises an Akt3 inhibitor. In some embodiments the Akt3 inhibitor is an Akt3 inhibitor disclosed in WO2016/102672.

In some embodiments of this aspect of the disclosure, the treatment is administered as a single agent. In other embodiments, the treatment is administered in combination with a further cancer treatment. Suitable further cancer treatments are outlined in detail above. In some preferred embodiments the further cancer treatment is a PD-1 inhibitor or PD-L1 inhibitor. In some embodiments the further cancer treatment is a monoclonal antibody such as pembrolizumab, alemtuzumab, bevacizumab, cetuximab, ibritumomab tiuxetan, or rituximab, or is a B-Raf inhibitor such as debrafenib, a MEK inhibitor such as trametinib or a combination therapy of these agents. In some particularly preferred embodiments the further cancer treatment is pembrolizumab. In other particularly preferred embodiments the further cancer treatment is a combination therapy of debrafenib and trametinib.

Kits

A fourth aspect of this facet of the present disclosure relates to diagnostic kits and test devices. In some embodiments the disclosure provides a diagnostic kit comprising 1, 2, 3, 4, 5, 6, 7, 8, 9, 10 or more reagents for detecting the activity, expression, or amount of one or more of the biomarkers described herein, each of which is selective for a particular biomarker. In some embodiments each of the reagents for detecting is selective for a biomarker selected from the group consisting of: Axl, IL-6r, HCC-4, TIMP-1, HB-EGF, ALP, LAP TGF-b1, EGF, PDGF-BB, OPG, NAP-2, ST2, TNF RI, VEGF, GRO-alpha, and/or LDH. In some embodiments each of the reagents for detecting is selective for a biomarker selected from the group consisting of: Axl, IL-6r, HCC-4, TIMP-1, HB-EGF, ALP, LAP TGF-b1, EGF, PDGF-BB, OPG, NAP-2, ST2, TNF RI, VEGF, and/or GRO-alpha.

In other embodiments the disclosure provides a test device comprising 1, 2, 3, 4, 5, 6, 7, 8, 9, 10 or more reagents for detecting the activity, expression, or amount of one or more of the biomarkers described herein, each of which is selective for a particular biomarker. In some embodiments each of the reagents for detecting is selective for a biomarker selected from the group consisting of: Axl, IL-6r, HCC-4, TIMP-1, HB-EGF, ALP, LAP TGF-b1, EGF, PDGF-BB, OPG, NAP-2, ST2, TNF RI, VEGF, GRO-alpha, and/or LDH. In some embodiments each of the reagents for detecting is selective for a biomarker selected from the group consisting of: Axl, IL-6r, HCC-4, TIMP-1, HB-EGF, ALP, LAP TGF-b1, EGF, PDGF-BB, OPG, NAP-2, ST2, TNF RI, VEGF, and/or GRO-alpha.

In some embodiments the diagnostic kit or test device comprises 1, 2, 3, 4, 5, 6, 7, 8, 9, 10 or more specific binding members, each of which selectively binds to one of the biomarkers described herein; and, one or more reagents for detecting said one or more specific binding members, or one or more reagents for detecting and/or quantifying formation of a complex formed by said specific binding member and said biomarker. In some embodiments each specific binding member selectively binds to a biomarker selected from the group consisting of: Axl, IL-6r, HCC-4, TIMP-1, HB-EGF, ALP, LAP TGF-b1, EGF, PDGF-BB, OPG, NAP-2, ST2, TNF RI, VEGF, GRO-alpha, and/or LDH. In some embodiments each specific binding member selectively binds to a biomarker selected from the group consisting of: Axl, IL-6r, HCC-4, TIMP-1, HB-EGF, ALP, LAP TGF-b1, EGF, PDGF-BB, OPG, NAP-2, ST2, TNF RI, VEGF, and/or GRO-alpha.

In some embodiments the diagnostic kit or test device comprises a specific binding member which selectively binds to Axl, and at least 1, 2, 3, 4, 5, 6, 7, 8, 9, 10, or more specific binding members, each of which selectively binds to a biomarker selected from the group consisting of: IL-6r, HCC-4, TIMP-1, HB-EGF, ALP, LAP TGF-b1, EGF, PDGF-BB, OPG, NAP-2, ST2, TNF RI, VEGF, GRO-alpha, and/or LDH. In some embodiments the diagnostic kit or test device comprises a specific binding member which selectively binds to LDH, and at least 1, 2, 3, 4, 5, 6, 7, 8, 9, 10, or more specific binding members, each of which selectively binds to a biomarker selected from the group consisting of: Axl, IL-6r, HCC-4, TIMP-1, HB-EGF, ALP, LAP TGF-b1, EGF, PDGF-BB, OPG, NAP-2, ST2, TNF RI, VEGF, and/or GRO-alpha.

In some embodiments the diagnostic kit or test device comprises a specific binding member which selectively binds to Axl, a specific binding member which selectively binds to LDH, and at least 1, 2, 3, 4, 5, 6, 7, 8, 9, 10, or more specific binding members, each of which selectively binds to a biomarker selected from the group consisting of: IL-6r, HCC-4, TIMP-1, HB-EGF, ALP, LAP TGF-b1, EGF, PDGF-BB, OPG, NAP-2, ST2, TNF RI, VEGF, and/or GRO-alpha.

In some embodiments of this aspect of the disclosure the diagnostic kit or test device comprises a plurality of said specific binding members provided in the form of an array on a substrate or conjugated to a plurality of particles, such as beads or microspheres. The particles may be encoded with a detectable label. In some embodiments the specific binding member comprises an antibody molecule or a binding fragment thereof.

In some embodiments of the diagnostic kit or test device detecting formation of a complex formed by a specific binding member and a biomarker is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.

In some embodiments of the diagnostic kit or test device, the kit or test device comprises no more than 15, 20, 25, 30, 40, or 50 reagents for detecting. In some embodiments of the diagnostic kit or test device, the kit or test device comprises no more than 15, 20, 25, 30, 40, or 50 specific binding members.

The present disclosure also provides use of such diagnostic kits and test devices in methods of predicting a cancer-related outcome in a subject. In preferred embodiments the cancer-related outcome is susceptibility to treatment with an agent capable of inhibiting or reversing EMT or a chemotherapeutic agent as described herein.

In some embodiments the agent capable of inhibiting or reversing EMT or chemotherapeutic agent is an Axl inhibitor, Akt3 inhibitor, Slfn11 inhibitor, or PHGDH inhibitor. In some preferred embodiments the agent capable of inhibiting or reversing EMT or chemotherapeutic agent is an Axl inhibitor, for example BGB324/R428/bemcentinib, cabozantinib, TP-0903, foretinib, merestinib, bosutinib, gilteritinib, crizotinib, amuvatinib, sunitinib, sitravatinib, LDC1267, or UNC2025. Other Axl inhibitors include the anti-Axl antibodies described in WO2015/193428, WO2015/193430, WO2016/097370, and WO2016/166296. In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In some embodiments of the diagnostic kit or test device for use in a predictive method, the subject has, is suspected of having, or has been diagnosed with melanoma.

In some embodiments the method may be as defined in accordance with the above described first and second aspects of this facet of the disclosure.

A fifth aspect of this facet of the present disclosure relates to the use of a reagent for detecting the activity, expression, or amount of a biomarker selected from the group consisting of: Axl, IL-6r, HCC-4, TIMP-1, HB-EGF, ALP, LAP TGF-b1, EGF, PDGF-BB, OPG, NAP-2, ST2, TNF RI, VEGF, GRO-alpha, and/or LDH, in a method of predicting a cancer-related outcome in a subject.

A sixth aspect of this facet of the disclosure relates to the use of 1, 2, 3, 4, 5, 6, 7, 8, 9, 10 or more reagents for detecting the activity, expression, or amount a biomarker, each of which is selective for a particular biomarker selected from the group consisting of: Axl, IL-6r, HCC-4, TIMP-1, HB-EGF, ALP, LAP TGF-b1, EGF, PDGF-BB, OPG, NAP-2, ST2, TNF RI, VEGF, GRO-alpha, and/or LDH, in a method of manufacture of a diagnostic kit or test device for use in a method of predicting a cancer-related outcome in a subject. In some embodiments of these uses the reagent for detecting comprises a specific binding member selective for a particular biomarker. The specific binding member may comprise an antibody molecule or a binding fragment thereof.

In some embodiments the method may be as defined in accordance with the above described first and second aspects of this facet of the disclosure.

Uses

A seventh aspect of this facet of the present disclosure relates to the use of one or more of: Axl, IL-6r, HCC-4, TIMP-1, HB-EGF, ALP, LAP TGF-b1, EGF, PDGF-BB, OPG, NAP-2, ST2, TNF RI, VEGF, GRO-alpha, and/or LDH, as a biomarker for determining if a subject is susceptible to treatment with an agent capable of inhibiting or reversing EMT or a chemotherapeutic agent.

In some embodiments the use relates to use of Axl, and at least 1, 2, 3, 4, 5, 6, 7, 8, 9, 10, or more of: IL-6r, HCC-4, TIMP-1, HB-EGF, ALP, LAP TGF-b1, EGF, PDGF-BB, OPG, NAP-2, ST2, TNF RI, VEGF, GRO-alpha, and/or LDH, as a biomarker for determining if a subject is susceptible to treatment with an agent capable of inhibiting or reversing EMT or a chemotherapeutic agent. In some embodiments the use relates to use of LDH, and at least 1, 2, 3, 4, 5, 6, 7, 8, 9, 10, or more of: Axl, IL-6r, HCC-4, TIMP-1, HB-EGF, ALP, LAP TGF-b1, EGF, PDGF-BB, OPG, NAP-2, ST2, TNF RI, VEGF, and/or GRO-alpha, as a biomarker for determining if a subject is susceptible to treatment with an agent capable of inhibiting or reversing EMT or a chemotherapeutic agent.

In some embodiments the use relates to use of Axl, LDH, and at least 1, 2, 3, 4, 5, 6, 7, 8, 9, 10, or more of: IL-6r, HCC-4, TIMP-1, HB-EGF, ALP, LAP TGF-b1, EGF, PDGF-BB, OPG, NAP-2, ST2, TNF RI, VEGF, and/or GRO-alpha, as a biomarker for determining if a subject is susceptible to treatment with an agent capable of inhibiting or reversing EMT or a chemotherapeutic agent.

In some embodiments the use is as a biomarker in a method as defined in accordance with the above described first and second aspects of this facet of the disclosure.

Methods of Treating a Subject

A eighth aspect of this facet of the present disclosure relates to methods of treating a subject having, suspected of having, or diagnosed with melanoma. In some embodiments the method comprises: identifying a subject for treatment using a predictive method defined in accordance with the above described first and second aspects of this facet of the disclosure; and, administering to the subject a therapeutically effective amount of an agent capable of inhibiting or reversing EMT or a chemotherapeutic agent.

Accordingly, in some embodiments, the method comprises: (a) obtaining a sample from the subject; (b) determining if the subject is susceptible to treatment with an agent capable of inhibiting or reversing EMT or a chemotherapeutic agent using a predictive method defined in accordance with the above described first and second aspects of this facet of the disclosure; and, (c) administering to the subject a therapeutically effective amount of an agent capable of inhibiting or reversing EMT or a chemotherapeutic agent.

In some embodiments the method comprises administering a therapeutically effective amount of an agent capable of inhibiting or reversing EMT or chemotherapeutic agent to a subject identified as susceptible to said treatment using a predictive method defined in accordance with the above described first and second aspects of this facet of the disclosure.

In other embodiments the method comprises: (a) administering to a subject a therapeutically effective amount of an agent capable of inhibiting or reversing EMT or a chemotherapeutic agent; and, (b) determining if the subject is susceptible to said treatment using a predictive method defined in accordance with the above described second aspect of this facet of the disclosure. In some embodiments the method further comprises: (c) administering to the subject one or more further therapeutically effective amounts of an agent capable of inhibiting or reversing EMT or a chemotherapeutic agent provided that the subject is identified as susceptible to said treatment.

A ninth aspect of this facet of the present disclosure relates to an agent capable of inhibiting or reversing EMT or chemotherapeutic agent for use in a method of treating a subject having, suspected of having, or diagnosed with melanoma. In some embodiments the method of treatment is a method as defined above.

A tenth aspect of this facet of the present disclosure relates to use of an agent capable of inhibiting or reversing EMT or chemotherapeutic agent for use in a method of manufacture of a medicament for use in a method of treating a subject having, suspected of having, or diagnosed with melanoma. In some embodiments the method of treatment is a method as defined above.

In some embodiments of these methods, agents for use, or uses, the agent capable of inhibiting or reversing EMT or chemotherapeutic agent comprises an Axl inhibitor, Akt3 inhibitor, Slfn11 inhibitor, or PHGDH inhibitor. In some preferred embodiments the agent capable of inhibiting or reversing EMT or chemotherapeutic agent is an Axl inhibitor, for example BGB324/R428/bemcentinib, cabozantinib, TP-0903, foretinib, merestinib, bosutinib, gilteritinib, crizotinib, amuvatinib, sunitinib, sitravatinib, LDC1267, or UNC2025. Other Axl inhibitors include the anti-Axl antibodies described in WO2015/193428, WO2015/193430, WO2016/097370, and WO2016/166296. In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In some other preferred embodiments, the treatment comprises an Akt3 inhibitor. In some embodiments the Akt3 inhibitor is an Akt3 inhibitor disclosed in WO2016/102672.

In some embodiments of these methods, agents for use, or uses, the treatment is administered as a single agent. In other embodiments, the treatment is administered in combination with a further cancer treatment. Suitable further cancer treatments are outlined in detail above. In some preferred embodiments the further cancer treatment is a PD-1 inhibitor or PD-L1 inhibitor. In some embodiments the further cancer treatment is a monoclonal antibody such as pembrolizumab, alemtuzumab, bevacizumab, cetuximab, ibritumomab tiuxetan, or rituximab, or is a B-Raf inhibitor such as debrafenib, a MEK inhibitor such as trametinib or a combination therapy of these agents. In some particularly preferred embodiments the further cancer treatment is pembrolizumab. In other particularly preferred embodiments the further cancer treatment is a combination therapy of debrafenib and trametinib.

Statements of Disclosure

The following numbered statements relate to aspects of this facet of the present disclosure, and form part of the description:
101. A method of predicting a cancer-related outcome in a subject, the method comprising assessing the activity, expression, or amount of one or more biomarker in the subject, or in a sample derived from the subject;
   wherein the one or more biomarker is selected from the group consisting of: Axl, IL-6r, HCC-4, TIMP-1, HB-EGF, ALP, LAP TGF-b1, EGF, PDGF-BB, OPG, NAP-2, ST2, TNF RI, VEGF, GRO-alpha, and/or LDH; and
   wherein the subject has, is suspected of having, or has been diagnosed with melanoma.
102. A method according to statement 101, wherein the method comprises:
   assessing the activity, expression, or amount of one or more biomarker in the subject, or in a sample derived from the subject, to obtain a sample profile of the one or more biomarker; and
   making a prediction based on the sample profile of the one or more biomarker.
103. A method according to statement 102, wherein the prediction is made by comparing the sample profile to a control profile.
104. A method according to statement 103, wherein the sample profile is obtained before the subject is contacted with or administered an agent capable of inhibiting or reversing EMT or a chemotherapeutic agent.
105. A method according to statement 104, wherein the control profile is:
   (i) obtained from a population of control subjects having melanoma; or
   (ii) obtained from a control subject or population of control subjects having melanoma and previously found to lack susceptibility to treatment with an agent capable of inhibiting or reversing EMT or a chemotherapeutic agent (that is, non-responder subjects);
   (iii) a predetermined profile of biomarker expression, activity, or amount values, for example a profile of “average, median, or mean” values or “standard ranges” of values, obtained from a control subject or population of control subjects having melanoma and previously found to lack susceptibility to treatment with an agent capable of inhibiting or reversing EMT or a chemotherapeutic agent (that is, non-responder subjects);
   (iv) obtained from a control sample having a known “average, median, or mean” value of biomarkers indicative of a control subject or population of control subjects having melanoma and previously found to lack susceptibility to treatment with an agent capable of inhibiting or reversing EMT or a chemotherapeutic agent (that is, non-responder subjects);
   (v) a predetermined profile of biomarker expression, activity, or amount “threshold” values obtained from a control subject or population of control subjects having melanoma and previously found to be susceptible to treatment with an agent capable of inhibiting or reversing EMT or a chemotherapeutic agent (that is, responder subjects); or
   (vi) obtained from a control sample having a known “threshold” value of biomarkers indicative of a control subject or population of control subjects having melanoma and previously found to be susceptible to treatment with an agent capable of inhibiting or reversing EMT or a chemotherapeutic agent (that is, responder subjects).
106. A method according to statement 105, wherein a higher activity, expression, or amount of one or more biomarker in the sample profile as compared with the control profile is indicative of susceptibility to treatment with an agent capable of inhibiting or reversing EMT or a chemotherapeutic agent.
107. A method according to statement 105, wherein a lower activity, expression, or amount of one or more biomarker in the sample profile as compared with the control profile is indicative of susceptibility to treatment with an agent capable of inhibiting or reversing EMT or a chemotherapeutic agent.
108. A method according to any one of statements 105-107, wherein the one or more biomarker includes at least one of: TIMP-1, HB-EGF, ALP, LAP TGF-b1, EGF, PDGF-BB, OPG, NAP-2, ST2, TNF RI, VEGF, GRO-alpha, and/or LDH,
   and wherein a higher activity, expression, or amount of one or more of said biomarkers in the sample profile as compared with the control profile is indicative of susceptibility to treatment with an agent capable of inhibiting or reversing EMT or a chemotherapeutic agent.
109. A method according to any one of statements 105-108, wherein the one or more biomarker includes at least one of: Axl, IL-6r, and/or HCC-4,
   and wherein a lower activity, expression, or amount of one or more of said biomarkers in the sample profile as compared with the control profile is indicative of susceptibility to treatment with an agent capable of inhibiting or reversing EMT or a chemotherapeutic agent.
110. A method according to any one of statements 105-109, wherein:
   a lower activity, expression, or amount of Axl; and/or
   a lower activity, expression, or amount of IL-6r; and/or
   a lower activity, expression, or amount of HCC-4; and/or
   a higher activity, expression, or amount of TIMP-1; and/or
   a higher activity, expression, or amount of HB-EGF; and/or
   a higher activity, expression, or amount of ALP; and/or
   a higher activity, expression, or amount of LAP TGF-b1; and/or
   a higher activity, expression, or amount of EGF; and/or
   a higher activity, expression, or amount of PDGF-BB; and/or
   a higher activity, expression, or amount of OPG; and/or
   a higher activity, expression, or amount of NAP-2; and/or
   a higher activity, expression, or amount of ST2; and/or
   a higher activity, expression, or amount of TNF RI; and/or
   a higher activity, expression, or amount of VEGF; and/or
   a higher activity, expression, or amount of GRO-alpha; and/or
   a higher activity, expression, or amount of LDH;
   in the sample profile as compared with the control profile is indicative of susceptibility to treatment with an agent capable of inhibiting or reversing EMT or a chemotherapeutic agent.
111. A method according to any one of statements 101-110, wherein the one or more biomarker comprises Axl, and/or LDH
   optionally wherein the one or more biomarker comprises Axl and at least 1, 2, 3, 4, 5, 6, 7, 8, 9, 10, or more further biomarkers selected from: IL-6r, HCC-4, TIMP-1, HB-EGF, ALP, LAP TGF-b1, EGF, PDGF-BB, OPG, NAP-2, ST2, TNF RI, VEGF, GRO-alpha, and/or LDH.
112. A method according to any one of statements 101-111, wherein:
   said Axl biomarker comprises an amino acid sequence having at least 70%, 80%, 90%, 95%, 99% or 100% sequence identity with the full-length of the amino acid sequence disclosed at UniProt accession no: P30530-1 or P30530-2 (entry version 196) or a fragment thereof;
   said Interleukin-6 receptor (IL-6r) biomarker comprises an amino acid sequence having at least 70%, 80%, 90%, 95%, 99% or 100% sequence identity with the full-length of the amino acid sequence disclosed at UniProt accession no: P08887-1 or P08887-2 (entry version 210) or a fragment thereof;
   said Chemokine CC-4 (HCC-4) biomarker comprises an amino acid sequence having at least 70%, 80%, 90%, 95%, 99% or 100% sequence identity with the full-length of the amino acid sequence disclosed at UniProt accession no: 015467 (entry version 146) or a fragment thereof;
   said Tissue Inhibitor of Metalloproteinases 1 (TIMP-1) biomarker comprises an amino acid sequence having at least 70%, 80%, 90%, 95%, 99% or 100% sequence identity with the full-length of the amino acid sequence disclosed at UniProt accession no: P01033 (entry version 202) or a fragment thereof;
   said Heparin-Binding EGF-Like Growth Factor (HB-EGF) biomarker comprises an amino acid sequence having at least 70%, 80%, 90%, 95%, 99% or 100% sequence identity with the full-length of the amino acid sequence disclosed at UniProt accession no: Q99075 (entry version 171) or a fragment thereof;
   said Antileukoproteinase (ALP) biomarker comprises an amino acid sequence having at least 70%, 80%, 90%, 95%, 99% or 100% sequence identity with the full-length of the amino acid sequence disclosed at UniProt accession no: P03973 (entry version 184) or a fragment thereof;
   said Latency-Associated Peptide of Transforming Growth Factor beta 1 (LAP TGF-b1) biomarker comprises an amino acid sequence having at least 70%, 80%, 90%, 95%, 99% or 100% sequence identity with the full-length of the amino acid sequence disclosed at UniProt accession no: P01137 (entry version 235) or a fragment thereof;
   said Epidermal Growth Factor (EGF) biomarker comprises an amino acid sequence having at least 70%, 80%, 90%, 95%, 99% or 100% sequence identity with the full-length of the amino acid sequence disclosed at UniProt accession no: P01133-1, P01133-2, or P01133-3 (entry version 204) or a fragment thereof;
   said Platelet-Derived Growth Factor BB (PDGF-BB) biomarker comprises an amino acid sequence having at least 70%, 80%, 90%, 95%, 99% or 100% sequence identity with the full-length of the amino acid sequence disclosed at UniProt accession no: P01127-1 or P01127-2 (entry version 217) or a fragment thereof;
   said Osteoprotegerin (OPG) biomarker comprises an amino acid sequence having at least 70%, 80%, 90%, 95%, 99% or 100% sequence identity with the full-length of the amino acid sequence disclosed at UniProt accession no: 000300 (entry version 155) or a fragment thereof;
   said Neutrophil Activating Peptide 2 (NAP-2) biomarker comprises an amino acid sequence having at least 70%, 80%, 90%, 95%, 99% or 100% sequence identity with the full-length of the amino acid sequence disclosed at UniProt accession no: P02775 (entry version 191) or a fragment thereof;
   said ST2 biomarker comprises an amino acid sequence having at least 70%, 80%, 90%, 95%, 99% or 100% sequence identity with the full-length of the amino acid sequence disclosed at UniProt accession no: Q01638-1, Q01638-2, Q01638-3, or Q01638-4 (entry version 179) or a fragment thereof;
   said Tumor Necrosis Factor Receptor I (TNF RI) biomarker comprises an amino acid sequence having at least 70%, 80%, 90%, 95%, 99% or 100% sequence identity with the full-length of the amino acid sequence disclosed at UniProt accession no: P19438-1, P19438-2, P19438-3, P19438-4, or P19438-5 (entry version 225) or a fragment thereof;
   said Vascular Endothelial Growth Factor (VEGF) biomarker comprises an amino acid sequence having at least 70%, 80%, 90%, 95%, 99% or 100% sequence identity with the full-length of the amino acid sequence disclosed at UniProt accession no: P15692-1, P15692-2, P15692-3, P15692-4, P15692-5, P15692-6, P15692-7, P15692-8, P15692-9, P15692-10, P15692-11, P15692-12, P15692-13, P15692-14, P15692-15, P15692-16, P15692-17, or P15692-18 (entry version 230) or a fragment thereof;
   said Growth-Regulated alpha protein (GRO-alpha) biomarker comprises an amino acid sequence having at least 70%, 80%, 90%, 95%, 99% or 100% sequence identity with the full-length of the amino acid sequence disclosed at UniProt accession no: P09341 (entry version 183) or a fragment thereof;
   said Lactate Dehydrogenase (LDH) biomarker comprises an amino acid sequence having at least 70%, 80%, 90%, 95%, 99% or 100% sequence identity with the full-length of the amino acid sequence disclosed at UniProt accession no: P00338-1, P00338-2, P00338-3, P00338-4, or P00338-5 (entry version 224) or a fragment thereof;
   said Lactate Dehydrogenase (LDH) biomarker comprises an amino acid sequence having at least 70%, 80%, 90%, 95%, 99% or 100% sequence identity with the full-length of the amino acid sequence disclosed at UniProt accession no: P07195-1 (entry version 218) or a fragment thereof;
      or a nucleic acid (either DNA or RNA) having at least 70%, 80%, 90%, 95%, 99% or 100% sequence identity with the full-length nucleic acid sequence encoding said amino acid sequences.
113. A method according to any one of statement 101-112, wherein the method comprises determining the amount of at least 2, 3, 4, 5, 6, 7, 8, 9, 10, 11, 12, 13, 14, 15, 20 of said biomarkers in the subject, or sample derived from the subject.
114. A method according to any one of statements 104-113, wherein assessing the activity, expression, or amount of one or more biomarker in the subject, or in a sample derived from the subject, comprises:
   contacting a sample, or an extract from the sample, with at least 1, 2, 3, 4, 5, 6, 7, 8, 9, 10 or more reagents for detecting the activity, expression, or amount of one or more biomarkers, each of which is selective for a particular biomarker; and
   detecting and/or quantifying said reagents for detecting.
115. A method according to statement 114, wherein assessing the expression or amount of one or more biomarker in the subject, or in a sample derived from the subject, comprises:
   contacting a sample, or an extract from the sample, with at least 1, 2, 3, 4, 5, 6, 7, 8, 9, 10 or more specific binding members, each of which selectively binds to a respective biomarker protein or mRNA; and
   detecting and/or quantifying formation of complex formed by said specific binding member and said biomarker protein or mRNA.
116. A method according to statement 115 wherein the specific binding member comprises an antibody molecule or binding fragment thereof.
117. A method according to statement 115 or 116, wherein detecting formation of a complex formed by the specific binding member and the biomarker protein or mRNA is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.
118. A method according to any one of statements 115-117 wherein the expression or amount of the one or more biomarker is determined as the absolute amount of the respective biomarker protein or mRNA in the sample.
119. A method according to any one of statements 115-117 wherein the expression or amount of the one or more biomarker is determined as a relative amount of the respective biomarker protein or mRNA in the sample,
   optionally wherein the relative amount is determined relative to a reference protein or mRNA in the sample, the expression of which is not altered by melanoma.
120. A method according to any one of statements 101-119, wherein the cancer-related outcome is susceptibility to treatment with an agent capable of inhibiting or reversing EMT or a chemotherapeutic agent.
121. A method according to statement 120, wherein the agent capable of inhibiting or reversing EMT or chemotherapeutic agent is an Axl inhibitor, Akt3 inhibitor, Slfn11 inhibitor, or PHGDH inhibitor.
122. A method according to statement 121, wherein the agent capable of inhibiting or reversing EMT or chemotherapeutic agent is an Axl inhibitor.
123. A method according to statement 122, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
124. A method according to statement 123, wherein the Axl inhibitor is BGB324/R428/bemcentinib.
125. A method according to statement 123, wherein the Axl inhibitor is an anti-Axl antibody described in WO2015/193428, WO2015/193430, WO2016/097370, or WO2016/166296.
126. A method according to statement 121, wherein the agent capable of inhibiting or reversing EMT or chemotherapeutic agent is an Akt3 inhibitor.
127. A method according to statement 126, wherein the Akt3 inhibitor is an Akt3 inhibitor disclosed in WO2016/102672.
128. A method according to any one of statements 120-127, wherein the agent capable of inhibiting or reversing EMT is administered as a single agent.
129. A method according to any one of statements 120-127, wherein the agent capable of inhibiting or reversing EMT is administered in combination with a further cancer treatment.
130. A method according to statement 129, wherein the further cancer treatment is selected from:
   (i) alkylating agents, including alkyl sulfonates such as busulfan;
   (ii) nitrogen mustards such as chlorambucil, cyclophosphamide, estramustine, ifosfamide, mechlorethamine, melphalan, and uramustine;
   (iii) ethyleneimine derivatives such as thiotepa;
   (iv) nitrosoureas such as carmustine, lomustine, and streptozocin;
   (v) triazenes such as dacarbazine, procarbazine, and temozolamide;
   (vi) platinum compounds such as cisplatin, carboplatin, oxaliplatin, satraplatin, and picoplatin onnaplatin, tetraplatin, sprioplatin, iproplatin, chloro(diethylenediamino)-platinum (II) chloride, dichloro(ethylenediamino)-platinum (II), diamino(2-ethylmalonato)platinum (II), (1,2-diaminocyclohexane)malonatoplatinum (II), (4-carboxyphthalo)-(1,2-diaminocyclohexane)platinum (II), (1,2-diaminocyclohexane)-(isocitrato)platinum (II), and (1,2-diaminocyclohexane)-cis-(pyruvato)platinum (II);
   (vii) antimetabolites, including antifolates such as methotrexate, permetrexed, raltitrexed, and trimetrexate;
   (viii) pyrimidine analogues such as azacitidine, capecitabine, cytarabine, edatrexate, floxuridine, fluorouracil, gemcitabine, and troxacitabine;
   (ix) purine analogues such as cladribine, chlorodeoxyadenosine, clofarabine, fludarabine, mercaptopurine, pentostatin, and thioguanine;
   (x) natural products, including antitumor antibiotics such as bleomycin, dactinomycin, mithramycin, mitomycin, mitoxantrone, porfiromycin;
   (xi) anthracyclines such as daunorubicin, doxorubicin, epirubicin, idarubicin, and valrubicin;
   (xii) mitotic inhibitors such as the vinca alkaloids vinblastine, vinvesir, vincristine, vindesine, and vinorelbine;
   (xiii) enzymes such as L-asparaginase and PEG-L-asparaginase;
   (xiv) microtubule polymer stabilizers such as the taxanes paclitaxel and docetaxel;
   (xv) topoisomerase I inhibitors such as the camptothecins irinotecan and topotecan;
   (xvi) topoisomerase II inhibitors such as podophyllotoxin, amsacrine, etoposide, teniposide, losoxantrone and actinomycin;
   (xvii) hormones and hormone antagonists, including
      androgens such as fluoxymesterone and testolactone;
      antiandrogens such as bicalutamide, cyproterone, flutamide, and nilutamide;
      corticosteroids such as dexamethasone and prednisone;
      aromatase inhibitors such as aminoglutethimide, anastrozole, exemestane, formestane, and letrozole;
      estrogens such as diethylstilbestrol;
      antiestrogens such as fulvestrant, raloxifene, tamoxifen, and toremifine;
      luteinising hormone-releasing hormone (LHRH) agonists and antagonists such as abarelix, buserelin, goserelin, leuprolide, histrelin, desorelin, nafarelin acetate and triptorelin;
      progestins such as medroxyprogesterone acetate and megestrol acetate; and
      thyroid hormones such as levothyroxine and liothyronine;
   (xviii) PKB pathway inhibitors, including perifosine, enzastaurin hydrochloride, and triciribine;
   (xix) P13K inhibitors such as semaphore and SF1126, and MTOR inhibitors such as rapamycin and analogues;
   (xx) CDK inhibitors, including seliciclib, alvocidib, and 7-hydroxystaurosporine;
   (xxi) COX-2 inhibitors, including celecoxib;
   (xxii) HDAC inhibitiors, including trichostatin A, suberoylanilide hydroxamic acid, and chlamydocin;
   (xxiii) DNA methylase inhibitors, including temozolomide, and miscellaneous agents, including altretamine, arsenic trioxide, thalidomide, lenalidomide, gallium nitrate, levamisole, mitotane, hydroxyurea, octreotide, procarbazine, suramin, photodynamic compounds such as methoxsalen and sodium porfimer;
   (xxiv) proteasome inhibitors such as bortezomib;
   (xxv) molecular targeted therapy agents including: functional therapeutic agents, including gene therapy agents and antisense therapy agents;
   (xxvi) tyrosine kinase inhibitors such as erlotinib hydrochloride, gefitinib, imatinib mesylate, and semaxanib;
   (xxvii) Raf inhibitors such as sorafenib, and gene expression modulators such as the retinoids and rexinoids, for example adapalene, bexarotene, trans-retinoic acid, 9-cis-retinoic acid, and N-(4-hydroxyphenyl)retinamide;
   (xxviii) phenotype-directed therapy agents, including monoclonal antibodies such as pembrolizumab, alemtuzumab, bevacizumab, cetuximab, ibritumomab tiuxetan, rituximab, and trastuzumab, immunotoxins such as gemtuzumab ozogamicin, radioimmunoconjugates such as I-tositumobab;
   (xxix) cancer vaccines;
   (xxx) biologic therapy agents including: interferons such as interferon-[alpha]2a and interferon-[alpha]2b, and interleukins such as aldesleukin, denileukin diftitox, and oprelvekin; (xxxi) anticancer therapies involving the use of protective or adjunctive agents, including: cytoprotective agents such as amifostine, and dexrazoxane, phosphonates such as pamidronate and zoledronic acid, and stimulating factors such as epoetin, darbeopetin, filgrastim, PEG-filgrastim, and sargramostim;
   (xxxii) Axl inhibitors such as 1-(6,7-dihydro-5H-benzo[6,7]cyclohepta[1,2-c]pyridazin-3-yl)-N3-((7-(S)-pyrrolidin-1-yl)-6,7,8,9-tetrahydro-5H-benzo[7]annulene-2-yl)-1H-1,2,4-triazole-3,5-diamine; and/or
   (xxxiii) further combination chemotherapeutic regimens, such as combinations of carboplatin/paclitaxel, capecitabine/docetaxel, fluorauracil/levamisole, fluorauracil/leucovorin, methotrexate/leucovorin, and trastuzumab/paclitaxel, alone or in further combination with carboplatin.
131. A method according to statement 129, wherein the further cancer treatment is a monoclonal antibody such as pembrolizumab, alemtuzumab, bevacizumab, cetuximab, ibritumomab tiuxetan, or rituximab, or is a B-Raf inhibitor such as debrafenib, a MEK inhibitor such as trametinib or a combination therapy of these agents.
132. A method according to statement 131, wherein the further cancer treatment is pembrolizumab.
133. A method according to statement 131, wherein the further cancer treatment is a combination therapy of debrafenib and trametinib.
134. A method according to any one of statements 101-133, wherein the subject is mammalian.
135. A method according to statement 134, wherein the subject is human.
136. A method according to any one of statements 101-135, wherein the predictive method is performed before the subject receives a treatment or course of treatment for melanoma.
137. A method according to any one of statement 101-136, wherein the predictive method is performed during or after a treatment or course of treatment for melanoma.
138. A method according to any one of statements 101-135 or 137, wherein the subject has previously been treated with an agent capable of inhibiting or reversing EMT or a chemotherapeutic agent.
139. A method according to statement 138, wherein the subject has previously been treated with an agent selected from: a PHGDH inhibitor, a Slfn11 inhibitor, an Axl inhibitor, or an Akt3 inhibitor.
140. A method according to any one of statements 138-139, wherein the subject has previously been treated with an agent that is different to the agent capable of inhibiting or reversing EMT or chemotherapeutic agent for which the subject's susceptibility is being predicted.
141. A method according to any one of statements 101-136, wherein the subject has not previously been treated with an agent capable of inhibiting or reversing EMT or a chemotherapeutic agent.
142. A method according to statement 141, wherein the subject has not previously been treated with an Axl inhibitor.
143. A method according to statement 141, wherein the subject has not previously been treated with an Akt3 inhibitor.
144. A method according to any one of statements 101-143, wherein the sample is selected from: blood, serum, plasma, tissue biopsy, culture supernatant, or urine.
145. A method according to statement 144, wherein the sample is a blood, serum, or plasma sample.
146. A method according to statement 145, wherein the sample is a serum sample.
147. A method according to any one of statements 104-146, wherein the method further comprises a subsequent step of assessing the activity, expression, or amount of one or more biomarker in the subject, in which a sample profile is obtained after contacting the subject with an agent capable of inhibiting or reversing EMT or a chemotherapeutic agent.
148. A method according to statement 103, wherein the sample profile is obtained after contacting the subject with an agent capable of inhibiting or reversing EMT or a chemotherapeutic agent.
149. A method according to statement 147 or 148, wherein the control profile is obtained from the same subject prior to contacting said subject with the agent capable of inhibiting or reversing EMT or a chemotherapeutic agent.
150. A method according to statement 149, wherein an increase in the activity, expression, or amount of one or more biomarker in the sample profile as compared with the control profile is indicative of higher susceptibility to treatment with an agent capable of inhibiting or reversing EMT or a chemotherapeutic agent.
151. A method according to statement 149, wherein a decrease in the activity, expression, or amount of one or more biomarker in the sample profile as compared with the control profile is indicative of higher susceptibility to treatment with an agent capable of inhibiting or reversing EMT or a chemotherapeutic agent.
152. A method according to statement 149, wherein an increase in the activity, expression, or amount of one or more biomarker in the sample profile as compared with the control profile is indicative of lower susceptibility to treatment with an agent capable of inhibiting or reversing EMT or a chemotherapeutic agent.
153. A method according to statement 149, wherein a decrease in the activity, expression, or amount of one or more biomarker in the sample profile as compared with the control profile is indicative of lower susceptibility to treatment with an agent capable of inhibiting or reversing EMT or a chemotherapeutic agent.
154. A method according to statement 150, wherein the one or more biomarker includes at least Axl.
155. A method according to any one of statements 147-154, wherein assessing the activity, expression, or amount of one or more biomarker in the subject, or in a sample derived from the subject, comprises:
   contacting a sample, or an extract from the sample, with at least 1, 2, 3, 4, 5, 6, 7, 8, 9, 10 or more reagents for detecting the activity, expression, or amount of one or more biomarkers, each of which is selective for a particular biomarker; and
   detecting and/or quantifying said reagents for detecting.
156. A method according to statement 155, wherein assessing the expression or amount of one or more biomarker in the subject, or in a sample derived from the subject, comprises:
   contacting a sample, or an extract from the sample, with at least 1, 2, 3, 4, 5, 6, 7, 8, 9, 10 or more specific binding members, each of which selectively binds to a respective biomarker protein or mRNA; and
   detecting and/or quantifying formation of a complex formed by said specific binding member and said biomarker protein or mRNA.
157. A method according to statement 156, wherein the specific binding member comprises an antibody molecule or binding fragment thereof.
158. A method according to statement 156 or 157, wherein detecting formation of a complex formed by the specific binding member and the biomarker protein or mRNA is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.
159. A method according to any one of statements 156-158 wherein the expression or amount of the one or more biomarker is determined as the absolute amount of the respective biomarker protein or mRNA in the sample.
160. A method according to any one of statements 156-158 wherein the expression or amount of the one or more biomarker is determined as a relative amount of the respective biomarker protein or mRNA in the sample,
   optionally wherein the relative amount is determined relative to a reference protein or mRNA in the sample, the expression of which is not altered by melanoma.
161. A method according to any one of statements 148-160, wherein the agent capable of inhibiting or reversing EMT or chemotherapeutic agent is an Axl inhibitor, Akt3 inhibitor, Slfn11 inhibitor, or PHGDH inhibitor.
162. A method according to statement 161, wherein the agent capable of inhibiting or reversing EMT or chemotherapeutic agent is an Axl inhibitor.
163. A method according to statement 162, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
164. A method according to statement 163, wherein the Axl inhibitor is BGB324/R428/bemcentinib.
165. A method according to statement 163, wherein the Axl inhibitor is an anti-Axl antibody described in WO2015/193428, WO2015/193430, WO2016/097370, or WO2016/166296.
166. A method according to statement 161, wherein the agent capable of inhibiting or reversing EMT or chemotherapeutic agent is an Akt3 inhibitor.
167. A method according to statement 166, wherein the Akt3 inhibitor is an Akt3 inhibitor disclosed in WO2016/102672.
168. A method according to any one of statements 161-167, wherein the agent capable of inhibiting or reversing EMT is administered as a single agent.
169. A method according to any one of statements 161-167, wherein the agent capable of inhibiting or reversing EMT is administered in combination with a further cancer treatment.
170. A method according to statement 169, wherein the further cancer treatment is selected from the further cancer treatments listed in statement 130.
171. A method according to statement 169, wherein the further cancer treatment is a monoclonal antibody such as pembrolizumab, alemtuzumab, bevacizumab, cetuximab, ibritumomab tiuxetan, or rituximab, or is a B-Raf inhibitor such as debrafenib, a MEK inhibitor such as trametinib or a combination therapy of these agents.
172. A method according to statement 171, wherein the further cancer treatment is pembrolizumab.
173. A method according to statement 171, wherein the further cancer treatment is a combination therapy of debrafenib and trametinib.
174. A method according to any one of statements 148-173, wherein the subject is mammalian.
175. A method according to statement 174, wherein the subject is human.
176. A method according to any one of statements 148-175, wherein the subject was selected for treatment with the agent capable of inhibiting or reversing EMT or a chemotherapeutic agent, using a predictive method according to any one of statements 101-147.
177. A method according to any one of statements 148-176, wherein the sample is selected from: blood, serum, plasma, tissue biopsy, culture supernatant, or urine.
178. A method according to statement 177, wherein the sample is a blood, serum, or plasma sample.
179. A method according to statement 178, wherein the sample is a serum sample.
180. A method according to any one of statements 1-179, wherein the method is performed in vitro or ex vivo.
201. A method of selecting subjects having, suspected of having, or diagnosed with melanoma for treatment with an agent capable of inhibiting or reversing EMT or a chemotherapeutic agent, the method comprising:
   identifying subjects susceptible to treatment with an agent capable of inhibiting or reversing EMT or a chemotherapeutic agent using a method according to any one of statements 101-180; and
   selecting thus identified subjects for treatment.
202. A method of selecting subjects having, suspected of having, or diagnosed with melanoma for continued treatment with an agent capable of inhibiting or reversing EMT or a chemotherapeutic agent, the method comprising:
   identifying subjects susceptible to treatment with an agent capable of inhibiting or reversing EMT or a chemotherapeutic agent using a method according to any one of statements 148-180; and selecting thus identified subjects for treatment.
203. A method according to any one of statements 201-202, wherein treatment comprises administering to the subject a therapeutically effective amount of an agent capable of inhibiting or reversing EMT or a chemotherapeutic agent.
204. A method according to statement 203, wherein the treatment comprises an Axl inhibitor, Akt3 inhibitor, Slfn11 inhibitor, or PHGDH inhibitor.
205. A method according to statement 204, wherein the treatment comprises an Axl inhibitor.
206. A method according to statement 205, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
207. A method according to statement 206, wherein the Axl inhibitor is BGB324/R428/bemcentinib.
208. A method according to statement 206, wherein the Axl inhibitor is an anti-Axl antibody described in WO2015/193428, WO2015/193430, WO2016/097370, or WO2016/166296.
209. A method according to statement 204, wherein the treatment comprises an Akt3 inhibitor.
210. A method according to statement 209, wherein the Akt3 inhibitor is an Akt3 inhibitor disclosed in WO2016/102672.
211. A method according to any one of statements 203-210, wherein the treatment is administered as a single agent.
212. A method according to any one of statements 203-211, wherein the treatment is administered in combination with a further cancer treatment.
213. A method according to statement 212, wherein the further cancer treatment is selected from the further cancer treatments listed in statement 130.
214. A method according to statement 212, wherein the further cancer treatment is a monoclonal antibody such as pembrolizumab, alemtuzumab, bevacizumab, cetuximab, ibritumomab tiuxetan, or rituximab, or is a B-Raf inhibitor such as debrafenib, a MEK inhibitor such as trametinib or a combination therapy of these agents.
215. A method according to statement 214, wherein the further cancer treatment is pembrolizumab.
216. A method according to statement 214, wherein the further cancer treatment is a combination therapy of debrafenib and trametinib.
301. A diagnostic kit comprising 1, 2, 3, 4, 5, 6, 7, 8, 9, 10 or more reagents for detecting the activity, expression, or amount of one or more biomarkers, each of which is selective for a particular biomarker selected from the group consisting of: Axl, IL-6r, HCC-4, TIMP-1, HB-EGF, ALP, LAP TGF-b1, EGF, PDGF-BB, OPG, NAP-2, ST2, TNF RI, VEGF, GRO-alpha, and/or LDH.
302. A test device comprising 1, 2, 3, 4, 5, 6, 7, 8, 9, 10 or more reagents for detecting the activity, expression, or amount of one or more biomarkers, each of which is selective for a particular biomarker selected from the group consisting of: Axl, IL-6r, HCC-4, TIMP-1, HB-EGF, ALP, LAP TGF-b1, EGF, PDGF-BB, OPG, NAP-2, ST2, TNF RI, VEGF, GRO-alpha, and/or LDH.
303. A diagnostic kit or test device according to any one of statements 301 or 302, comprising:
   1, 2, 3, 4, 5, 6, 7, 8, 9, 10 or more specific binding members, each of which selectively binds to a biomarker selected from the group consisting of: Axl, IL-6r, HCC-4, TIMP-1, HB-EGF, ALP, LAP TGF-b1, EGF, PDGF-BB, OPG, NAP-2, ST2, TNF RI, VEGF, GRO-alpha, and/or LDH; and
   one or more reagents for detecting said one or more specific binding members or one or more reagents for detecting and/or quantifying formation of a complex formed by said specific binding member and said biomarker.
304. A diagnostic kit or test device according to statement 303, wherein the specific binding member comprises an antibody molecule or binding fragment thereof.
305. A diagnostic kit or test device according to statement 303 or 304, wherein detecting formation of a complex formed by the specific binding member and the biomarker is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.
306. A diagnostic kit or test device according to any one of statements 301-305, wherein the kit or test device comprises no more than 15, 20, 25, 30, 40, or 50 reagents for detecting.
307. A diagnostic kit or test device according to any one of statements 301-306 for use in a method of predicting a cancer-related outcome in a subject.
308. A diagnostic kit for use, or test device for use, according to statement 307, wherein the cancer-related outcome is susceptibility to treatment with an agent capable of inhibiting or reversing EMT or a chemotherapeutic agent.
309. A diagnostic kit for use, or test device for use, according to statement 308, wherein the agent capable of inhibiting or reversing EMT or chemotherapeutic agent is an Axl inhibitor, Akt3 inhibitor, Slfn11 inhibitor, or PHGDH inhibitor.
310. A diagnostic kit for use, or test device for use, according to statement 308, wherein the agent capable of inhibiting or reversing EMT or chemotherapeutic agent is an Axl inhibitor.
311. A diagnostic kit for use, or test device for use, according to statement 310, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
312. A diagnostic kit for use, or test device for use, according to statement 311, wherein the Axl inhibitor is BGB324/R428/bemcentinib.
313. A diagnostic kit for use, or test device for use, according to statement 311, wherein the Axl inhibitor is an anti-Axl antibody described in WO2015/193428, WO2015/193430, WO2016/097370, or WO2016/166296.
314. A diagnostic kit for use, or test device for use, according to statement 308, wherein the agent capable of inhibiting or reversing EMT or chemotherapeutic agent is an Akt3 inhibitor.
315. A diagnostic kit for use, or test device for use, according to any one of statements 307-310, wherein the subject has, is suspected of having, or has been diagnosed with melanoma.
316. A diagnostic kit for use, or test device for use, according to any one of statements 307-315, wherein the method is a method according to any one of statements 101-180.
317. Use of a reagent for detecting the activity, expression, or amount of a biomarker selected from the group consisting of: Axl, IL-6r, HCC-4, TIMP-1, HB-EGF, ALP, LAP TGF-b1, EGF, PDGF-BB, OPG, NAP-2, ST2, TNF RI, VEGF, GRO-alpha, and/or LDH in a method of predicting a cancer-related outcome in a subject.
318. Use of 1, 2, 3, 4, 5, 6, 7, 8, 9, 10 or more reagents for detecting the activity, expression, or amount a biomarker, each of which is selective for a particular biomarker selected from the group consisting of: Axl, IL-6r, HCC-4, TIMP-1, HB-EGF, ALP, LAP TGF-b1, EGF, PDGF-BB, OPG, NAP-2, ST2, TNF RI, VEGF, GRO-alpha, and/or LDH, in a method of manufacture of a diagnostic kit or test device for use in a method of predicting a cancer-related outcome in a subject.
319. A use according to statement 317 or 318, wherein the reagent for detecting comprises a specific binding member selective for a particular biomarker.
320. A use according to statement 317 or 318, wherein the method of predicting a cancer-related outcome in a subject is a method according to any one of statements 101-180.
401. Use of one or more of: Axl, IL-6r, HCC-4, TIMP-1, HB-EGF, ALP, LAP TGF-b1, EGF, PDGF-BB, OPG, NAP-2, ST2, TNF RI, VEGF, GRO-alpha, and/or LDH as a biomarker for determining if a subject is susceptible to treatment with an agent capable of inhibiting or reversing EMT or a chemotherapeutic agent.
402. A use according to statement 401, wherein:
   (i) a higher activity, expression, or amount of one or more of said biomarkers in the subject, or sample derived from the subject, compared with a control profile for the respective biomarker is indicative of susceptibility to treatment with an agent capable of inhibiting or reversing EMT or a chemotherapeutic agent; or
   (ii) a lower activity, expression, or amount of one or more of said biomarkers in the subject, or sample derived from the subject, compared with a control profile for the respective biomarker is indicative of susceptibility to treatment with an agent capable of inhibiting or reversing EMT or a chemotherapeutic agent.
403. A use according to statement 401, wherein:
   (i) an increase in the activity, expression, or amount of one or more biomarker relative to a control profile for the respective biomarker after contacting the subject with a cancer therapeutic agent is indicative of increased susceptibility to treatment with an agent capable of inhibiting or reversing EMT or a chemotherapeutic agent; or
   (ii) a decrease in the activity, expression, or amount of one or more biomarker relative to a control profile for the respective biomarker after contacting the subject with a cancer therapeutic agent is indicative of increased susceptibility to treatment with an agent capable of inhibiting or reversing EMT or a chemotherapeutic agent.
404. A use according to any one of statements 401-403, wherein the use is as a biomarker in a method according to any one of statements 101-180
501. A method of treating a subject having, suspected of having, or diagnosed with melanoma, the method comprising:
   identifying a subject susceptible to treatment using a method according to any one of statements 101-180; and
   administering to the subject a therapeutically effective amount of an agent capable of inhibiting or reversing EMT or a chemotherapeutic agent.
502. A method of treating a subject having, suspected of having, or diagnosed with melanoma, the method comprising:
   (a) obtaining a sample from the subject;
   (b) determining if the subject is susceptible to treatment with an agent capable of inhibiting or reversing EMT or a chemotherapeutic agent using a method according to any one of statements 101-180;
   (c) administering to the subject a therapeutically effective amount of an agent capable of inhibiting or reversing EMT or a chemotherapeutic agent.
503. A method of treating a subject having, suspected of having, or diagnosed with melanoma, the method comprising administering a therapeutically effective amount of an agent capable of inhibiting or reversing EMT or chemotherapeutic agent to a subject identified as susceptible to said treatment using a method according to any one of statements 101-180.
504. A method of treating a subject having, suspected of having, or diagnosed with melanoma, the method comprising:
   administering to the subject a therapeutically effective amount of an agent capable of inhibiting or reversing EMT or a chemotherapeutic agent; and
   determining if the subject is susceptible to said treatment using a method according to any one of statements 148-180.
505. A method according to statement 504, wherein the method comprises administering to the subject one or more further therapeutically effective amounts of an agent capable of inhibiting or reversing EMT or a chemotherapeutic agent provided that the subject is identified as susceptible to said treatment.
506. An agent capable of inhibiting or reversing EMT or chemotherapeutic agent for use in a method of treating a subject having, suspected of having, or diagnosed with melanoma,
   wherein the method is a method according to any one of statements 501-505.
507. Use of an agent capable of inhibiting or reversing EMT or chemotherapeutic agent for use in a method of manufacture of a medicament for use in a method of treating a subject having, suspected of having, or diagnosed with melanoma,
   wherein the method of treatment is a method according to any one of statements 501-505.
508. A method, agent for use, or use according to any one of statements 501-507, wherein the agent capable of inhibiting or reversing EMT or chemotherapeutic agent comprises an Axl inhibitor, Akt3 inhibitor, Slfn11 inhibitor, or PHGDH inhibitor.
509. A method, agent for use, or use according to statement 508, wherein the agent capable of inhibiting or reversing EMT or chemotherapeutic agent comprises an Axl inhibitor.
510. A method, agent for use, or use according to statement 509, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
511. A method, agent for use, or use according to statement 510, wherein the Axl inhibitor is BGB324/R428/bemcentinib.
512. A method, agent for use, or use according to statement 510, wherein the Axl inhibitor is an anti-Axl antibody described in WO2015/193428, WO2015/193430, WO2016/097370, or WO2016/166296.
512. A method, agent for use, or use according to statement 508, wherein the agent capable of inhibiting or reversing EMT or chemotherapeutic agent comprises an Akt3 inhibitor.
513. A method, agent for use, or use according to statement 512, wherein the Akt3 inhibitor is an Akt3 inhibitor disclosed in WO2016/102672.
514. A method, agent for use, or use according to any one of statements 501-513, wherein the agent capable of inhibiting or reversing EMT or chemotherapeutic agent is administered as a single agent.
515. A method, agent for use, or use according to any one of statements 501-513, wherein the agent capable of inhibiting or reversing EMT or chemotherapeutic agent is administered in combination with a further cancer treatment.
516. A method, agent for use, or use according to statement 515, wherein the further cancer treatment is selected from the further cancer treatments listed in statement 130.
517. A method, agent for use, or use according to statement 515, wherein the further cancer treatment is a monoclonal antibody such as pembrolizumab, alemtuzumab, bevacizumab, cetuximab, ibritumomab tiuxetan, or rituximab, or is a B-Raf inhibitor such as debrafenib, a MEK inhibitor such as trametinib or a combination therapy of these agents.
518. A method, agent for use, or use according to statement 517, wherein the further cancer treatment is pembrolizumab.
519. A method, agent for use, or use according to statement 517, wherein the further cancer treatment is a combination therapy of debrafenib and trametinib.

Facet 4a: Melanoma

Melanoma, also known as malignant melanoma, is a class of cancer that develops from melanocytes. These cancers typically occur in the skin, but can also occur in the mouth, intestines, or eye. Chemotherapeutic drug therapies for melanoma include, for example ipilimumab, pembrolizumab, and nivolumab. The overall success rate of such treatments in metastatic melanoma remains limited, and there remains a need for targeted therapies for this cancer type (Hsueh et al, 2016).

Accordingly, robust biomarkers for predicting cancer-related outcomes in a subject having melanoma, for example susceptibility to treatment with an agent capable of inhibiting or reversing EMT or a chemotherapeutic agent, would be useful in identifying subjects who are most likely to respond to or benefit from treatment with a particular agent/regimen.

A “responder” subject as described herein is a subject who is likely to, or who does, respond to treatment (or benefit from treatment) with an agent capable of inhibiting or reversing EMT or a chemotherapeutic agent, for example the small molecule Axl kinase inhibitor BGB324/R428/bemcentinib when administered either as a single agent or in combination with another cancer treatment.

Correspondingly, a “non-responder” subject as described herein is a subject who is not likely to, or who does not, respond to treatment (or benefit from treatment) with an agent capable of inhibiting or reversing EMT or a chemotherapeutic agent, for example the small molecule Axl kinase inhibitor BGB324/R428/bemcentinib when administered either as a single agent or in combination with another cancer treatment.

To “respond to treatment” or “benefit from treatment” as used herein means to experience an overall clinical benefit from the treatment. This overall clinical benefit can be any of: prolonged survival, partial or complete disease remission, slowing or absence of disease progression, tumour shrinkage (for example, a reduction in tumour volume of 5, 10, 20, 30, 40% or more), reduction in tumour burden (for example, a reduction in tumour burden of 5, 10, 20, 30, 40% or more), slowing or absence of tumour enlargement, slowing or absence of increase in tumour burden, improved quality of life, progression-free survival, overall survival, or any other positive patient outcome. Suitable methods for determining tumour volume/burden are well known to the skilled person, for example, using: computed tomography (CT), or magnetic resonance imaging (MRI) imaging technologies; X-ray imaging, for example, mammography; ultrasound imaging; nuclear imaging, for example positron emission tomography (PET), PET/CT scans, bone scans, gallium scans, or metaiodobenzylguanidine (MIBG) scans; bioluminescence imaging (BLI); fluorescence imaging (FLI); BD ToF (infrared-based 3D Time-of-Flight camera) imaging.

Biomarkers

The term “marker” or “biomarker” is used herein to refer to a gene or protein whose expression in a subject or sample derived from a subject is altered or modulated, for example, up or down regulated, in cancer. Where the biomarker is a protein, modulation or alteration of expression encompasses modulation through different post-translational modifications.

In accordance with any aspect of this facet of the present disclosure the biomarkers described herein may be defined as follows, wherein:
an Axl biomarker comprises an amino acid sequence having at least 70%, 80%, 90%, 95%, 99% or 100% sequence identity with the full-length of the amino acid sequence disclosed at UniProt accession no: P30530-1 or P30530-2 (entry version 196) or a fragment thereof;
a Alpha-Fetoprotein (AFP) biomarker comprises an amino acid sequence having at least 70%, 80%, 90%, 95%, 99% or 100% sequence identity with the full-length of the amino acid sequence disclosed at UniProt accession no: P02771-1 (entry version 192) or a fragment thereof;
a Heparin-Binding EGF-Like Growth Factor (HB-EGF) biomarker comprises an amino acid sequence having at least 70%, 80%, 90%, 95%, 99% or 100% sequence identity with the full-length of the amino acid sequence disclosed at UniProt accession no: Q99075 (entry version 171) or a fragment thereof;
a Neuronal Cell Adhesion Molecule (Nr-CAM) biomarker comprises an amino acid sequence having at least 70%, 80%, 90%, 95%, 99% or 100% sequence identity with the full-length of the amino acid sequence disclosed at UniProt accession no: Q92823-1, Q92823-2, Q92823-3, Q92823-4, Q92823-5, or Q92823-6 (entry version 172) or a fragment thereof;
a Kidney Injury Molecule-1 (KIM-1) biomarker comprises an amino acid sequence having at least 70%, 80%, 90%, 95%, 99% or 100% sequence identity with the full-length of the amino acid sequence disclosed at UniProt accession no: Q96D42-1 (entry version 141) or a fragment thereof;
a CD 40 antigen (CD40) comprises an amino acid sequence having at least 70%, 80%, 90%, 95%, 99% or 100% sequence identity with the full-length of the amino acid sequence disclosed at UniProt accession no: P25942-1 or P25942-2 (entry version 221) or a fragment thereof;
a Chromogranin-A (CgA) biomarker comprises an amino acid sequence having at least 70%, 80%, 90%, 95%, 99% or 100% sequence identity with the full-length of the amino acid sequence disclosed at UniProt accession no: P10645-1 (entry version 200) or a fragment thereof;
a Interleukin-2 receptor alpha (IL-2 receptor alpha) biomarker comprises an amino acid sequence having at least 70%, 80%, 90%, 95%, 99% or 100% sequence identity with the full-length of the amino acid sequence disclosed at UniProt accession no: P01589-1 (entry version 200) or a fragment thereof;
a Human Chorionic Gonadotropin beta (hCG) comprises an amino acid sequence having at least 70%, 80%, 90%, 95%, 99% or 100% sequence identity with the full-length of the amino acid sequence disclosed at UniProt accession no: PODN86-1 or PODN86-2 (entry version 26) or a fragment thereof;
a Latency-Associated Peptide of Transforming Growth Factor beta 1 (LAP TGF-b1) biomarker comprises an amino acid sequence having at least 70%, 80%, 90%, 95%, 99% or 100% sequence identity with the full-length of the amino acid sequence disclosed at UniProt accession no: P01137 (entry version 235) or a fragment thereof;
a Thymus and activation-regulated chemokine (TARC) biomarker comprises an amino acid sequence having at least 70%, 80%, 90%, 95%, 99% or 100% sequence identity with the full-length of the amino acid sequence disclosed at UniProt accession no: Q92583-1 (entry version 163) or a fragment thereof;
a CD40 Ligand (CD40-L) biomarker comprises an amino acid sequence having at least 70%, 80%, 90%, 95%, 99% or 100% sequence identity with the full-length of the amino acid sequence disclosed at UniProt accession no: P29965 (entry version 199) or a fragment thereof;
a Epidermal Growth Factor (EGF) biomarker comprises an amino acid sequence having at least 70%, 80%, 90%, 95%, 99% or 100% sequence identity with the full-length of the amino acid sequence disclosed at UniProt accession no: P01133-1, P01133-2, or P01133-3 (entry version 204) or a fragment thereof;
a Vascular endothelial growth factor receptor 3 (VEGFR-3) biomarker comprises an amino acid sequence having at least 70%, 80%, 90%, 95%, 99% or 100% sequence identity with the full-length of the amino acid sequence disclosed at UniProt accession no: P35916-1, P35916-2, or P35916-3 (entry version 206) or a fragment thereof;
a Pancreatic secretory trypsin inhibitor (TATI) biomarker comprises an amino acid sequence having at least 70%, 80%, 90%, 95%, 99% or 100% sequence identity with the full-length of the amino acid sequence disclosed at UniProt accession no: P00995-1 (entry version 193) or a fragment thereof;
a Matrix Metalloproteinase-7 (MMP-7) biomarker comprises an amino acid sequence having at least 70%, 80%, 90%, 95%, 99% or 100% sequence identity with the full-length of the amino acid sequence disclosed at UniProt accession no: P09237-1 (entry version 197) or a fragment thereof;
a Leptin biomarker comprises an amino acid sequence having at least 70%, 80%, 90%, 95%, 99% or 100% sequence identity with the full-length of the amino acid sequence disclosed at UniProt accession no: P41159 (entry version 176) or a fragment thereof;
a Apolipoprotein A-I (Apo A-I) biomarker comprises an amino acid sequence having at least 70%, 80%, 90%, 95%, 99% or 100% sequence identity with the full-length of the amino acid sequence disclosed at UniProt accession no: P02647-1 (entry version 241) or a fragment thereof;
a Periostin biomarker comprises an amino acid sequence having at least 70%, 80%, 90%, 95%, 99% or 100% sequence identity with the full-length of the amino acid sequence disclosed at UniProt accession no: Q15063-1, Q15063-2, Q15063-3, Q15063-4, Q15063-5, Q15063-6, Q15063-7 (entry version 160) or a fragment thereof;
a Angiopoietin-2 (ANG-2) biomarker comprises an amino acid sequence having at least 70%, 80%, 90%, 95%, 99% or 100% sequence identity with the full-length of the amino acid sequence disclosed at UniProt accession no: 015123-1, 015123-2, or 015123-3 (entry version 163) or a fragment thereof;
a Cystatin-C biomarker comprises an amino acid sequence having at least 70%, 80%, 90%, 95%, 99% or 100% sequence identity with the full-length of the amino acid sequence disclosed at UniProt accession no: P01034-1 (entry version 213) or a fragment thereof;
a Hepsin biomarker comprises an amino acid sequence having at least 70%, 80%, 90%, 95%, 99% or 100% sequence identity with the full-length of the amino acid sequence disclosed at UniProt accession no: P05981-1 (entry version 183) or a fragment thereof;
a Apolipoprotein C-I (Apo C-I) biomarker comprises an amino acid sequence having at least 70%, 80%, 90%, 95%, 99% or 100% sequence identity with the full-length of the amino acid sequence disclosed at UniProt accession no: P02654 (entry version 175) or a fragment thereof;
a Interleukin-1 receptor type 1 (IL-1RI) biomarker comprises an amino acid sequence having at least 70%, 80%, 90%, 95%, 99% or 100% sequence identity with the full-length of the amino acid sequence disclosed at UniProt accession no: P14778-1 (entry version 215) or a fragment thereof;
a Eotaxin-2 biomarker comprises an amino acid sequence having at least 70%, 80%, 90%, 95%, 99% or 100% sequence identity with the full-length of the amino acid sequence disclosed at UniProt accession no: 000175-1 (entry version 162) or a fragment thereof;
a Myeloperoxidase (MPO) biomarker comprises an amino acid sequence having at least 70%, 80%, 90%, 95%, 99% or 100% sequence identity with the full-length of the amino acid sequence disclosed at UniProt accession no: P05164-1, P05164-2, or P05164-3 (entry version 217) or a fragment thereof;
a Ferritin (FRTN) biomarker comprises an amino acid sequence having at least 70%, 80%, 90%, 95%, 99% or 100% sequence identity with the full-length of the amino acid sequence disclosed at UniProt accession no: P02794 (entry version 213) or a fragment thereof;
a Lactate Dehydrogenase (LDH) biomarker comprises an amino acid sequence having at least 70%, 80%, 90%, 95%, 99% or 100% sequence identity with the full-length of the amino acid sequence disclosed at UniProt accession no: P00338-1, P00338-2, P00338-3, P00338-4, or P00338-5 (entry version 224) or a fragment thereof; and/or
a Lactate Dehydrogenase (LDH) biomarker comprises an amino acid sequence having at least 70%, 80%, 90%, 95%, 99% or 100% sequence identity with the full-length of the amino acid sequence disclosed at UniProt accession no: P07195-1 (entry version 218) or a fragment thereof.

The biomarkers described herein may also be defined as a nucleic acid (either DNA or RNA) having at least 70%, 80%, 90%, 95%, 99% or 100% sequence identity with the full-length nucleic acid sequence encoding any of the above amino acid sequences.

Identity may be as defined using sequence comparisons made using FASTA and FASTP (see Pearson & Lipman, 1988. Methods in Enzymology 183: 63-98). Parameters are preferably set, using the default matrix, as follows: Gapopen (penalty for the first residue in a gap): −12 for proteins/-16 for DNA; Gapext (penalty for additional residues in a gap): −2 for proteins/-4 for DNA; KTUP word length: 2 for proteins/6 for DNA.

Pre-Treatment Predictive Biomarkers and Methods

By analysing patient data obtained from clinical trial BGBIL006 (NCT02872259), the authors have discovered that, prior to treatment of subjects having melanoma, certain of the biomarkers described herein are present in the serum of “responder” subjects at higher levels than in the serum of “non-responder” subjects—see Example 2 and Example 7. Similarly, prior to treatment of the subject certain of the biomarkers described herein are present in the serum of “responder” subjects at lower levels than in the serum of “non-responder” subjects. Thus, by assessing the activity, expression, or amount of one or more of the biomarkers described herein prior to treatment with an agent capable of inhibiting or reversing EMT or a chemotherapeutic agent it is possible to determine the likelihood of that subject being susceptible or responsive to treatment with said agent.

Accordingly, in a first aspect of this facet, the disclosure provides a method of predicting a cancer-related outcome in a subject having, suspected of having, or diagnosed with melanoma, the method comprising assessing the activity, expression, or amount of one or more of the biomarkers described herein in the subject, or in a sample derived from the subject.

In some embodiments the one or more biomarker is selected from the group consisting of: HB-EGF, AFP, CgA, Nr-CAM, KIM-1, CD40, LAP TGF-b1, EGF, IL-2 receptor alpha, hCG, TARO, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH.

A “cancer-related outcome” as described herein is a clinical prediction or prognosis associated with cancer. In some embodiments the cancer-related outcome is susceptibility to treatment with an agent capable of inhibiting or reversing EMT or a chemotherapeutic agent.

In some embodiments the method comprises assessing the activity, expression, or amount of one or more of the biomarkers described herein in the subject, or in a sample derived from the subject, to obtain a sample profile of the one or more biomarker; and, making a prediction based on the sample profile of the one or more biomarker.

A “sample profile” as used herein is a profile of numerical values or numerical ranges corresponding to the activity, expression, or amount of respective biomarkers determined in the subject or sample derived from the subject.

In some embodiments of the method the prediction is made by comparing the sample profile to a control profile. A “control profile” as used herein is a profile of numerical values or numerical ranges corresponding to the activity, expression, or amount of the same biomarkers as for the sample profile, determined in a control subject or population of control subjects. Suitable control profiles are described in more detail below.

Prior to treatment of a subject, certain of the biomarkers described herein have been found to exhibit up-regulated expression in “responder” subjects—that is, certain of the biomarkers described herein have been found to be present in the serum of “responder” subjects at higher levels than in the serum of “non-responder” subjects. Similarly, prior to treatment of a subject, certain of the biomarkers described herein have been found to exhibit down-regulated expression in “responder” subjects—that is, certain of the biomarkers described herein have been found to be present in the serum of “responder” subjects at lower levels than in the serum of “non-responder” subjects.

This difference in expression level between “responder” and “non-responder” subjects can also be expressed in the opposite way. That is to say, prior to treatment of a subject, certain of the biomarkers described herein have been found to exhibit down-regulated expression in “non-responder” subjects—that is, certain of the biomarkers described herein have been found to be present in the serum of “non-responder” subjects at lower levels than in the serum of “responder” subjects. Similarly, prior to treatment of a subject, certain of the biomarkers described herein have been found to exhibit up-regulated expression in “non-responder” subjects—that is, certain of the biomarkers described herein have been found to be present in the serum of “non-responder” subjects at higher levels than in the serum of “responder” subjects.

The discovery of these differential patterns of expression mean that, by assessing the activity, expression, or amount of one or more of the biomarkers described herein prior to treatment of a subject with an agent, it is possible to determine the likelihood of that subject being susceptible or responsive to treatment with said agent. Accordingly, in particularly preferred embodiments the sample profile is obtained before the subject is contacted with or administered the agent capable of inhibiting or reversing EMT or a chemotherapeutic agent.

A subject who is “susceptible to treatment” with an agent capable of inhibiting or reversing EMT or a chemotherapeutic agent is one who is likely to, or who does, respond to treatment (or benefit from treatment) with an agent capable of inhibiting or reversing EMT or a chemotherapeutic agent. That is, a subject who is “susceptible to treatment” with an agent is a “responder” subject for that agent, as defined above. A prediction that is “indicative of susceptibility” is one which suggests or points towards the likelihood that a subject will respond to or benefit from treatment.

In some embodiments the predictive methods of the disclosure comprise assessing the activity, expression, or amount of one or more of the biomarkers described herein in the subject, or in a sample derived from the subject, to obtain a sample profile of the one or more biomarker; and, making a prediction based on the sample profile of the one or more biomarker. In some embodiments of the method the prediction is made by comparing the sample profile to a control profile, wherein the control profile is a profile of numerical values or numerical ranges corresponding to the activity, expression, or amount of the same biomarkers as for the sample profile, determined in a control subject or population of control subjects.

In some embodiments the control profile may be obtained from a population of control subjects having melanoma. In some embodiments the control profile may be obtained from a population of control subjects not having melanoma. In other embodiments the control profile may be obtained from a control subject or population of control subjects having melanoma and previously found to lack susceptibility to treatment with an agent capable of inhibiting or reversing EMT or a chemotherapeutic agent (that is, non-responder subjects).

In some embodiments the control profile may a predetermined profile of biomarker expression, activity, or amount values, for example a profile of “mean” values, “threshold” values, or “standard ranges” of values. This predetermined profile of biomarker expression, activity, or amount values can be obtained, for example, from a control subject or population of control subjects having melanoma, a control subject or population of control subjects not having melanoma, or a control subject or population of control subjects having melanoma and previously found to lack susceptibility to treatment with an agent capable of inhibiting or reversing EMT or a chemotherapeutic agent (that is, non-responder subjects).

In some other embodiments the control profile may be obtained from a control subject or population of control subjects having melanoma and previously found to be susceptible to treatment with an agent capable of inhibiting or reversing EMT or a chemotherapeutic agent (that is, responder subjects). In some embodiments the control profile may a predetermined profile of biomarker expression, activity, or amount values, for example a profile of “threshold”, values or “standard ranges” of values. This predetermined profile of biomarker expression, activity, or amount values can be obtained, for example, from a control subject or population of control subjects having melanoma and previously found to be susceptible to treatment with an agent capable of inhibiting or reversing EMT or a chemotherapeutic agent (that is, responder subjects).

In some other embodiments the control profile may be obtained from a control sample having a known amount (for example a “threshold” amount) of the same biomarkers as will be determined in the sample profile. This threshold amount can be obtained, for example, from non-responder or responder subjects as outlined above.

The skilled person is readily able to determine suitable “mean”, “threshold”, or “standard range” values for a particular biomarker.

For example, a “mean” value may be determined by: assessing the activity, expression, or amount of a particular biomarker in a population of subjects having (or not having) melanoma; and, averaging the biomarker activity, expression, or amount determined to determine a “mean” value.

For example, a “mean” value may be determined by: assessing the activity, expression, or amount of a particular biomarker in a population of subjects; determining which subjects in the population are susceptible to treatment with an agent capable of inhibiting or reversing EMT or a chemotherapeutic agent; and, averaging the biomarker activity, expression, or amount determined in subjects found to be susceptible to treatment with an agent capable of inhibiting or reversing EMT or a chemotherapeutic agent (that is, responder subjects) to determine a “mean” value.

For example, a “threshold” value may be determined by: assessing the activity, expression, or amount of a particular biomarker in a population of subjects; determining which subjects in the population are susceptible to treatment with an agent capable of inhibiting or reversing EMT or a chemotherapeutic agent; and, selecting as appropriate the highest or lowest biomarker activity, expression, or amount determined in a subject found to be susceptible to treatment with an agent capable of inhibiting or reversing EMT or a chemotherapeutic agent (that is, a responder subject) as the “threshold value”.

For example, a “standard range” of values may be determined by: assessing the activity, expression, or amount of a particular biomarker in a population of subjects having (or not having) melanoma; and, determining a “standard range” of values based on this assessment.

For example, a “standard range” of values may be determined by: assessing the activity, expression, or amount of a particular biomarker in a population of subjects; determining which subjects in the population are susceptible to treatment with an agent capable of inhibiting or reversing EMT or a chemotherapeutic agent; and, determining a “standard range” of values for subjects found to be susceptible to treatment with an agent capable of inhibiting or reversing EMT or a chemotherapeutic agent (that is, a responder subject) based on this assessment.

Where the control profile is a predetermined profile of biomarker expression, activity, or amount values, this may be provided as a “look-up table” or data record. Predetermined control profiles of this type may be a profile of numerical values or numerical ranges corresponding to a measure of central tendency (such as an average, median or mean) amount of the respective biomarkers determined in a plurality of control subjects (for example non-responder or responder subjects as outlined above). The skilled person is readily able to determine both a measure of central tendency and a measure of variability within a population using standard protein quantification techniques and suitable statistical tools.

Accordingly, in embodiments in which the control profile is: obtained from a population of control subjects having melanoma; obtained from a population of control subjects not having melanoma; obtained from a control subject or population of control subjects having melanoma and previously found to lack susceptibility to treatment with an agent capable of inhibiting or reversing EMT or a chemotherapeutic agent (that is non-responder subjects); a predetermined profile of biomarker expression, activity, or amount values (for example a profile of “average, median, or mean” values or “standard ranges” of values) obtained from non-responder subjects; obtained from a control sample having a known “average, median, or mean” value of biomarkers indicative of non-responder subjects; a predetermined profile of biomarker expression, activity, or amount “threshold” values obtained from responder subjects; or, obtained from a control sample having a known “threshold” value of biomarkers indicative of responder subjects; then, a higher activity, expression, or amount of one or more of the biomarkers described herein in a sample profile as compared with the control profile may be indicative of susceptibility to treatment with an agent capable of inhibiting or reversing EMT or a chemotherapeutic agent. Similarly, a lower activity, expression, or amount of one or more of the biomarkers described herein in the sample profile as compared with a control profile may be indicative of susceptibility to treatment with an agent capable of inhibiting or reversing EMT or a chemotherapeutic agent.

In some such embodiments, the method may comprise assessing the activity, expression, or amount of one or more of biomarker selected from: HB-EGF, CgA, Nr-CAM, KIM-1, CD40, LAP TGF-b1, EGF, IL-2 receptor alpha, TARC, CD40-L, VEGFR-3, TATI, MMP-7, Leptin, Apo A-I, ANG-2, Cystatin-C, Hepsin Apo C-I, IL-1RI, Axl, and/or LDH; wherein a higher activity, expression, or amount of one or more of said biomarkers in the sample profile as compared with the control profile is indicative of susceptibility to treatment with an agent capable of inhibiting or reversing EMT or a chemotherapeutic agent.

In some such embodiments, the method may comprise assessing the activity, expression, or amount of one or more of biomarker selected from: AFP, Periostin, hCG, Eotaxin-2, MPO, and/or FRTN; wherein a lower activity, expression, or amount of one or more of said biomarkers in the sample profile as compared with the control profile is indicative of susceptibility to treatment with an agent capable of inhibiting or reversing EMT or a chemotherapeutic agent.

In some such embodiments: a lower activity, expression, or amount of AFP; and/or a lower activity, expression, or amount of Periostin; and/or a lower activity, expression, or amount of hCG; and/or a lower activity, expression, or amount of Eotaxin-2; and/or a lower activity, expression, or amount of MPO; and/or a lower activity, expression, or amount of FRTN; and/or a higher activity, expression, or amount of TIMP-1; and/or a higher activity, expression, or amount of HB-EGF; and/or a higher activity, expression, or amount of HB-EGF; and/or a higher activity, expression, or amount of CgA; and/or a higher activity, expression, or amount of Nr-CAM; and/or a higher activity, expression, or amount of KIM-1; and/or a higher activity, expression, or amount of CD40; and/or a higher activity, expression, or amount of LAP TGF-b1; and/or a higher activity, expression, or amount of EGF; and/or a higher activity, expression, or amount of IL-2ra; and/or a higher activity, expression, or amount of TARO; and/or a higher activity, expression, or amount of CD40-L; and/or a higher activity, expression, or amount of VEGFR-3; and/or a higher activity, expression, or amount of TATI; and/or a higher activity, expression, or amount of MMP-7; and/or a higher activity, expression, or amount of Leptin; and/or a higher activity, expression, or amount of Apo A-I; and/or a higher activity, expression, or amount of ANG-2; and/or a higher activity, expression, or amount of Cystatin-C; and/or a higher activity, expression, or amount of Hepsin; and/or a higher activity, expression, or amount of Apo C-I; and/or a higher activity, expression, or amount of IL-1RI; and/or a higher activity, expression, or amount of Axl; and/or a higher activity, expression, or amount of LDH in the sample profile as compared with the control profile is indicative of susceptibility to treatment with an agent capable of inhibiting or reversing EMT or a chemotherapeutic agent.

In embodiments in which the control profile is: obtained from a control subject or population of control subjects having melanoma and previously found to be susceptible to treatment with an agent capable of inhibiting or reversing EMT or a chemotherapeutic agent (that is, responder subjects); then, if the expression, activity, or amount values in the sample profile are above a “threshold” value typical of a responder subject (that is, a subject having melanoma and previously found to be susceptible to treatment with an agent capable of inhibiting or reversing EMT or a chemotherapeutic agent) this is indicative of susceptibility to treatment with an agent capable of inhibiting or reversing EMT or a chemotherapeutic agent. Similarly, if the expression, activity, or amount values in the sample profile are below the “threshold” value typical of a responder subject (that is, a subject having melanoma and previously found to be susceptible to treatment with an agent capable of inhibiting or reversing EMT or a chemotherapeutic agent) this is indicative of a lack of susceptibility to treatment with an agent capable of inhibiting or reversing EMT or a chemotherapeutic agent.

In embodiments in which the control profile is: a predetermined profile of “standard ranges” of biomarker expression, activity, or amount values obtained from responder subjects; then, if the expression, activity, or amount values in the sample profile are within the “standard range” of values typical of a responder subject (that is, a subject having melanoma and previously found to be susceptible to treatment with an agent capable of inhibiting or reversing EMT or a chemotherapeutic agent) this is indicative of susceptibility to treatment with an agent capable of inhibiting or reversing EMT or a chemotherapeutic agent. Similarly, if the expression, activity, or amount values in the sample profile are outside the “standard range” of values typical of a responder subject (that is, a subject having melanoma and previously found to be susceptible to treatment with an agent capable of inhibiting or reversing EMT or a chemotherapeutic agent) this is indicative of a lack of susceptibility to treatment with an agent capable of inhibiting or reversing EMT or a chemotherapeutic agent.

In embodiments in which the control profile is: a predetermined profile of “standard ranges” of biomarker expression, activity, or amount values obtained from non-responder subjects; then, if the expression, activity, or amount values in the sample profile are within the “standard range” of values typical of a non-responder subject (that is, a subject having melanoma and previously found to lack susceptibility to treatment with an agent capable of inhibiting or reversing EMT or a chemotherapeutic agent) this is indicative of a lack of susceptibility to treatment with an agent capable of inhibiting or reversing EMT or a chemotherapeutic agent. Similarly, if the expression, activity, or amount values in the sample profile are outside the “standard range” of values typical of a non-responder subject (that is, a subject having melanoma and previously found to lack susceptibility to treatment with an agent capable of inhibiting or reversing EMT or a chemotherapeutic agent) this may be indicative of susceptibility to treatment with an agent capable of inhibiting or reversing EMT or a chemotherapeutic agent.

In embodiments in which the control profile is: obtained from a population of control subjects not having melanoma; then, if the expression, activity, or amount values in the sample profile are outside a “standard range” of values typical of subjects not having melanoma, this is indicative of susceptibility to treatment with an agent capable of inhibiting or reversing EMT or a chemotherapeutic agent. Similarly, if the expression, activity, or amount values in the sample profile are within a “standard range” of values typical of subjects not having melanoma, this is indicative of a lack of susceptibility to treatment with an agent capable of inhibiting or reversing EMT or a chemotherapeutic agent. In such cases, an expression, activity or amount value may be outside the “standard range” of values when it is either above the upper limit of normal or below the lower limit of normal for a given biomarker. The upper limit of normal and lower limit of normal are the limits of the normal distribution of a given biomarker in a population of control subjects not having melanoma.

In embodiments in which the one or more biomarker comprises LDH, an expression or amount of LDH higher than 287 units per litre (U/L) may be indicative of susceptibility to treatment with an agent capable of inhibiting or reversing EMT or a chemotherapeutic agent.

The skilled person is readily able to determine whether the sample profile is indicative of susceptibility or lack of susceptibility to treatment with an agent capable of inhibiting or reversing EMT or a chemotherapeutic agent using suitable statistical tools and comparison with the appropriate control.

In some embodiments the sample profile may be compared with two or more control profiles as described above.

As used herein, the term “one or more” means 1, 2, 3, 4, 5, 6, 7, 8, 9, 10, 11, 12, 13, 14, 15, 16, 17, 18, 19, or 20 of the biomarkers described herein. In some embodiments the one or more biomarker is HB-EGF. In some embodiments the one or more biomarker is AFP. In some embodiments the one or more biomarker is Axl. In some embodiments the one or more biomarker is CgA. In some embodiments the one or more biomarker is Nr-CAM. In some embodiments the one or more biomarker is KIM-1. In some embodiments the one or more biomarker is CD40. In some embodiments the one or more biomarker is LAP TGF-b1. In some embodiments the one or more biomarker is EGF. In some embodiments the one or more biomarker is IL-2 receptor alpha. In some embodiments the one or more biomarker is hCG. In some embodiments the one or more biomarker is TARO. In some embodiments the one or more biomarker is CD40-L. In some embodiments the one or more biomarker is VEGFR-3. In some embodiments the one or more biomarker is TATI. In some embodiments the one or more biomarker is MMP-7. In some embodiments the one or more biomarker is Leptin. In some embodiments the one or more biomarker is Apo A-I. In some embodiments the one or more biomarker is Periostin. In some embodiments the one or more biomarker is ANG-2. In some embodiments the one or more biomarker is Cystatin-C. In some embodiments the one or more biomarker is Hepsin. In some embodiments the one or more biomarker is Apo C-I. In some embodiments the one or more biomarker is IL-1RI. In some embodiments the one or more biomarker is Eotaxin-21n some embodiments the one or more biomarker is MPO. In some embodiments the one or more biomarker is FRTN. In some embodiments the one or more biomarker is LDH.

In some preferred embodiments the one or more biomarker is HB-EGF. In some preferred embodiments the one or more biomarker is Axl. In some preferred embodiments the one or more biomarker is AFP. In some preferred embodiments the one or more biomarker is CgA. In some preferred embodiments the one or more biomarker is Nr-CAM. In some preferred embodiments the one or more biomarker is KIM-1. In some preferred embodiments the one or more biomarker is CD40. In some preferred embodiments the one or more biomarker is LAP TGF-b1. In some preferred embodiments the one or more biomarker is EGF. In some preferred embodiments the one or more biomarker is TATI.

In some embodiments the one or more biomarker is HB-EGF and AFP. In some embodiments the one or more biomarker is HB-EGF and CgA. In some embodiments the one or more biomarker is HB-EGF and Nr-CAM. In some embodiments the one or more biomarker is HB-EGF and TATI. In some embodiments the one or more biomarker is HB-EGF and KIM-1. In some embodiments the one or more biomarker is HB-EGF and CD40. In some embodiments the one or more biomarker is HB-EGF and LAP TGF-b1. In some embodiments the one or more biomarker is HB-EGF and EGF. In some embodiments the one or more biomarker is HB-EGF and Axl.

In some embodiments the one or more biomarker is AFP and CgA. In some embodiments the one or more biomarker is AFP and Nr-CAM. In some embodiments the one or more biomarker is AFP and TATI. In some embodiments the one or more biomarker is AFP and KIM-1. In some embodiments the one or more biomarker is AFP and CD40. In some embodiments the one or more biomarker is AFP and LAP TGF-b1. In some embodiments the one or more biomarker is AFP and EGF. In some embodiments the one or more biomarker is AFP and Axl.

In some embodiments the one or more biomarker is CgA and Nr-CAM. In some embodiments the one or more biomarker is CgA and TATI. In some embodiments the one or more biomarker is CgA and KIM-1. In some embodiments the one or more biomarker is CgA and CD40. In some embodiments the one or more biomarker is CgA and LAP TGF-b1. In some embodiments the one or more biomarker is CgA and EGF. In some embodiments the one or more biomarker is CgA and Axl.

In some embodiments the one or more biomarker is Nr-CAM and TATI. In some embodiments the one or more biomarker is Nr-CAM and KIM-1. In some embodiments the one or more biomarker is Nr-CAM and CD40. In some embodiments the one or more biomarker is Nr-CAM and LAP TGF-b1. In some embodiments the one or more biomarker is Nr-CAM and EGF. In some embodiments the one or more biomarker is Nr-CAM and Axl.

In some embodiments the one or more biomarker is TATI and KIM-1. In some embodiments the one or more biomarker is TATI and CD40. In some embodiments the one or more biomarker is TATI and LAP TGF-b1. In some embodiments the one or more biomarker is TATI and EGF. In some embodiments the one or more biomarker is TATI and Axl.

In some embodiments the one or more biomarker is KIM-1 and CD40. In some embodiments the one or more biomarker is KIM-1 and LAP TGF-b1. In some embodiments the one or more biomarker is KIM-1 and EGF. In some embodiments the one or more biomarker is KIM-1 and Axl.

In some embodiments the one or more biomarker is CD40 and LAP TGF-b1. In some embodiments the one or more biomarker is CD40 and EGF. In some embodiments the one or more biomarker is CD40 and Axl.

In some embodiments the one or more biomarker is EGF and LAP TGF-b1. In some embodiments the one or more biomarker is EGF and Axl.

In some embodiments the one or more biomarker is Axl and LAP TGF-b1

In some embodiments the one or more biomarker is CgA, HB-EGF and AFP. In some embodiments the one or more biomarker is CgA, HB-EGF and Nr-CAM. In some embodiments the one or more biomarker is CgA, HB-EGF and TATI. In some embodiments the one or more biomarker is CgA, HB-EGF and KIM-1. In some embodiments the one or more biomarker is CgA, HB-EGF and CD40. In some embodiments the one or more biomarker is CgA, HB-EGF and LAP TGF-b1. In some embodiments the one or more biomarker is CgA, HB-EGF and EGF. In some embodiments the one or more biomarker is CgA, HB-EGF and Axl.

In some embodiments the one or more biomarker is CgA, AFP and Nr-CAM. In some embodiments the one or more biomarker is CgA, AFP and TATI. In some embodiments the one or more biomarker is CgA, AFP and KIM-1. In some embodiments the one or more biomarker is CgA, AFP and CD40. In some embodiments the one or more biomarker is CgA, AFP and LAP TGF-b1. In some embodiments the one or more biomarker is CgA, AFP and EGF. In some embodiments the one or more biomarker is CgA, AFP and Axl.

In some embodiments the one or more biomarker is CgA, Nr-CAM and TATI. In some embodiments the one or more biomarker is CgA, Nr-CAM and KIM-1. In some embodiments the one or more biomarker is CgA, Nr-CAM and CD40. In some embodiments the one or more biomarker is CgA, Nr-CAM and LAP TGF-b1. In some embodiments the one or more biomarker is CgA, Nr-CAM and EGF. In some embodiments the one or more biomarker is CgA, Nr-CAM and Axl.

In some embodiments the one or more biomarker is CgA, TATI and KIM-1. In some embodiments the one or more biomarker is CgA, TATI and CD40. In some embodiments the one or more biomarker is CgA, TATI and LAP TGF-b1. In some embodiments the one or more biomarker is CgA, TATI and EGF. In some embodiments the one or more biomarker is CgA, TATI and Axl.

In some embodiments the one or more biomarker is CgA, KIM-1 and CD40. In some embodiments the one or more biomarker is CgA, KIM-1 and LAP TGF-b1. In some embodiments the one or more biomarker is CgA, KIM-1 and EGF. In some embodiments the one or more biomarker is CgA, KIM-1 and Axl.

In some embodiments the one or more biomarker is CgA, CD40 and LAP TGF-b1. In some embodiments the one or more biomarker is CgA, CD40 and EGF. In some embodiments the one or more biomarker is CgA, CD40 and Axl.

In some embodiments the one or more biomarker is CgA, EGF and LAP TGF-b1. In some embodiments the one or more biomarker is CgA, EGF and Axl.

In some embodiments the one or more biomarker is CgA, Axl, and LAP TGF-b1

In some embodiments the one or more biomarker is HB-EGF and AFP. In some embodiments the one or more biomarker is HB-EGF and CgA. In some embodiments the one or more biomarker is HB-EGF and Nr-CAM. In some embodiments the one or more biomarker is HB-EGF and TATI. In some embodiments the one or more biomarker is HB-EGF and KIM-1. In some embodiments the one or more biomarker is HB-EGF and CD40. In some embodiments the one or more biomarker is HB-EGF and LAP TGF-b1. In some embodiments the one or more biomarker is HB-EGF and EGF. In some embodiments the one or more biomarker is HB-EGF and Axl.

In some embodiments the one or more biomarker is AFP, Nr-CAM and TATI. In some embodiments the one or more biomarker is AFP, Nr-CAM and KIM-1. In some embodiments the one or more biomarker is Nr-CAM and CD40. In some embodiments the one or more biomarker is AFP, Nr-CAM and LAP TGF-b1. In some embodiments the one or more biomarker is AFP, Nr-CAM and EGF. In some embodiments the one or more biomarker is AFP, Nr-CAM and Axl.

In some embodiments the one or more biomarker is AFP, TATI and KIM-1. In some embodiments the one or more biomarker is AFP, TATI and CD40. In some embodiments the one or more biomarker is AFP, TATI and LAP TGF-b1. In some embodiments the one or more biomarker is AFP, TATI and EGF. In some embodiments the one or more biomarker is AFP, TATI and Axl.

In some embodiments the one or more biomarker is AFP, KIM-1 and CD40. In some embodiments the one or more biomarker is AFP, KIM-1 and LAP TGF-b1. In some embodiments the one or more biomarker is AFP, KIM-1 and EGF. In some embodiments the one or more biomarker is AFP, KIM-1 and Axl.

In some embodiments the one or more biomarker is AFP, CD40 and LAP TGF-b1. In some embodiments the one or more biomarker is AFP, CD40 and EGF. In some embodiments the one or more biomarker is AFP, CD40 and Axl.

In some embodiments the one or more biomarker is AFP, EGF and LAP TGF-b1. In some embodiments the one or more biomarker is AFP, EGF and Axl.

In some embodiments the one or more biomarker is AFP, Axl and LAP TGF-b1

In some embodiments the one or more biomarker is HB-EGF, Nr-CAM and TATI. In some embodiments the one or more biomarker is HB-EGF, Nr-CAM and KIM-1. In some embodiments the one or more biomarker is HB-EGF, Nr-CAM and CD40. In some embodiments the one or more biomarker is HB-EGF, Nr-CAM and LAP TGF-b1. In some embodiments the one or more biomarker is HB-EGF, Nr-CAM and EGF. In some embodiments the one or more biomarker is HB-EGF, Nr-CAM and Axl.

In some embodiments the one or more biomarker is HB-EGF, TATI and KIM-1. In some embodiments the one or more biomarker is HB-EGF, TATI and CD40. In some embodiments the one or more biomarker is HB-EGF, TATI and LAP TGF-b1. In some embodiments the one or more biomarker is HB-EGF, TATI and EGF. In some embodiments the one or more biomarker is HB-EGF, TATI and Axl.

In some embodiments the one or more biomarker is HB-EGF, KIM-1 and CD40. In some embodiments the one or more biomarker is HB-EGF, KIM-1 and LAP TGF-b1. In some embodiments the one or more biomarker is HB-EGF, KIM-1 and EGF. In some embodiments the one or more biomarker is HB-EGF, KIM-1 and Axl.

In some embodiments the one or more biomarker is HB-EGF, CD40 and LAP TGF-b1. In some embodiments the one or more biomarker is HB-EGF, CD40 and EGF. In some embodiments the one or more biomarker is HB-EGF, CD40 and Axl.

In some embodiments the one or more biomarker is HB-EGF, EGF and LAP TGF-b1. In some embodiments the one or more biomarker is HB-EGF, EGF and Axl.

In some embodiments the one or more biomarker is HB-EGF, Axl and LAP TGF-b1

In some embodiments the one or more biomarker is Nr-CAM, TATI and KIM-1. In some embodiments the one or more biomarker is Nr-CAM, TATI and CD40. In some embodiments the one or more biomarker is Nr-CAM, TATI and LAP TGF-b1. In some embodiments the one or more biomarker is Nr-CAM, TATI and EGF. In some embodiments the one or more biomarker is Nr-CAM, TATI and Axl.

In some embodiments the one or more biomarker is Nr-CAM, KIM-1 and CD40. In some embodiments the one or more biomarker is Nr-CAM, KIM-1 and LAP TGF-b1. In some embodiments the one or more biomarker is Nr-CAM, Nr-CAM, KIM-1 and EGF. In some embodiments the one or more biomarker is Nr-CAM, KIM-1 and Axl.

In some embodiments the one or more biomarker is Nr-CAM, CD40 and LAP TGF-b1. In some embodiments the one or more biomarker is Nr-CAM, CD40 and EGF. In some embodiments the one or more biomarker is Nr-CAM, CD40 and Axl.

In some embodiments the one or more biomarker is Nr-CAM, EGF and LAP TGF-b1. In some embodiments the one or more biomarker is Nr-CAM, EGF and Axl.

In some embodiments the one or more biomarker is Nr-CAM, Axl and LAP TGF-b1

In some embodiments the one or more biomarker is TATI, KIM-1 and CD40. In some embodiments the one or more biomarker is TATI, KIM-1 and LAP TGF-b1. In some embodiments the one or more biomarker is TATI, KIM-1 and EGF. In some embodiments the one or more biomarker is TATI, KIM-1 and Axl.

In some embodiments the one or more biomarker is TATI, CD40 and LAP TGF-b1. In some embodiments the one or more biomarker is TATI, CD40 and EGF. In some embodiments the one or more biomarker is TATI, CD40 and Axl.

In some embodiments the one or more biomarker is TATI, EGF and LAP TGF-b1. In some embodiments the one or more biomarker is TATI, EGF and Axl.

In some embodiments the one or more biomarker is TATI, Axl and LAP TGF-b1

In some embodiments the one or more biomarker is KIM-1, CD40 and LAP TGF-b1. In some embodiments the one or more biomarker is KIM-1, CD40 and EGF. In some embodiments the one or more biomarker is KIM-1, CD40 and Axl.

In some embodiments the one or more biomarker is KIM-1, EGF and LAP TGF-b1. In some embodiments the one or more biomarker is KIM-1, EGF and Axl.

In some embodiments the one or more biomarker is KIM-1, Axl and LAP TGF-b1

In some embodiments the one or more biomarker is CD40, EGF and LAP TGF-b1. In some embodiments the one or more biomarker is CD40, EGF and Axl.

In some embodiments the one or more biomarker is CD40, Axl and LAP TGF-b1

In some embodiments the one or more biomarker is EGF, Axl and LAP TGF-b1

In some embodiments the method may comprise assessing the activity, expression, or amount of at least 2, 3, 4, 5, 6, 7, 8, 9, 10, 11, 12, 13, 14, 15, 20 of said biomarkers in the subject, or in a sample derived from the subject. In some embodiments the method may comprise assessing the activity, expression, or amount of Axl and at least 1, 2, 3, 4, 5, 6, 7, 8, 9, 10, 11, 12, 13, 14, 15, 20 further biomarkers described herein in the subject, or in a sample derived from the subject. In some embodiments the method may comprise assessing the activity, expression, or amount of LDH and at least 1, 2, 3, 4, 5, 6, 7, 8, 9, 10, 11, 12, 13, 14, 15, 20 further biomarkers described herein in the subject, or in a sample derived from the subject. In some embodiments the method may comprise assessing the activity, expression, or amount of Axl, LDH, and at least 1, 2, 3, 4, 5, 6, 7, 8, 9, 10, 11, 12, 13, 14, 15, 20 further biomarkers described herein in the subject, or in a sample derived from the subject.

Subject

A “subject” as referred to herein is any species of animal classified as a mammal, and includes but is not restricted to, domestic and farm animals, primates and humans. In some preferred embodiments the subject is a human of any sex or race. In some embodiments, the human is an adult human.

In some embodiments the predictive methods described herein are performed before the subject receives a treatment or course of treatment for melanoma. In other embodiments the predictive method is performed during or after a treatment or course of treatment for melanoma. In some embodiments the treatment or course of treatment for melanoma is an agent capable of inhibiting or reversing EMT. In other embodiments the treatment or course of treatment for melanoma is a chemotherapeutic treatment that is not an agent capable of inhibiting or reversing EMT.

In some embodiments of the predictive methods described herein the subject has previously been treated with an agent capable of inhibiting or reversing EMT or a chemotherapeutic agent. In some embodiments the subject has previously been treated with an agent selected from: a PHGDH inhibitor, a Slfn11 inhibitor, an Axl inhibitor, or an Akt3 inhibitor.

In some embodiments of the predictive methods described herein the subject has previously been treated with an agent that is different to the agent capable of inhibiting or reversing EMT or chemotherapeutic agent for which the subject's response is being predicted. That is, the subject has previously been treated with an agent that is not the agent which the predictive method is determining the subject's susceptibility to. Accordingly, in some embodiments, the subject has not previously been treated with an agent capable of inhibiting or reversing EMT or a chemotherapeutic agent. In some preferred embodiments, the subject has not previously been treated with an Axl inhibitor. In other preferred embodiments, the subject has not previously been treated with an Akt3 inhibitor.

Post-Treatment Changes in Biomarkers

In some embodiments of this aspect of the disclosure, the predictive method further comprises a subsequent step of assessing the activity, expression, or amount of one or more biomarker in the subject, or in a sample derived from the subject, to obtain a second sample profile of one or more of the biomarkers described herein after the subject has received a treatment or course of treatment an agent capable of inhibiting or reversing EMT or a chemotherapeutic agent.

That is, in some embodiments, the predictive method comprises:
(a) determining if a subject is susceptible to treatment with an agent capable of inhibiting or reversing EMT or a chemotherapeutic agent by assessing the activity, expression, or amount of one or more biomarker in the subject, as outlined above;
(b) administering to the subject a therapeutically effective amount of an agent capable of inhibiting or reversing EMT or a chemotherapeutic agent; and
(c) subsequently assessing the activity, expression, or amount of one or more biomarker in the subject, or in a sample derived from the subject.

In some embodiments, the predictive method comprises:
(a) determining if a subject is susceptible to treatment with an agent capable of inhibiting or reversing EMT or a chemotherapeutic agent by assessing the activity, expression, or amount of one or more biomarker in the subject, or in a sample derived from the subject, wherein the one or more biomarker is selected from the group consisting of: HB-EGF, AFP, CgA, Nr-CAM, KIM-1, CD40, LAP TGF-b1, EGF, IL-2 receptor alpha, hCG, TARO, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH;
(b) administering to the subject a therapeutically effective amount of an agent capable of inhibiting or reversing EMT or a chemotherapeutic agent; and
(c) subsequently assessing the activity, expression, or amount of one or more biomarker in the subject, or in a sample derived from the subject.

In some preferred embodiments the methods comprise subsequently assessing the activity, expression, or amount of HB-EGF, AFP, CgA, Nr-CAM, KIM-1, CD40, LAP TGF-b1, EGF, Axl, and/or LDH in the subject, or in a sample derived from the subject. In some embodiments an increase in the activity, expression, or amount of HB-EGF, AFP, CgA, Nr-CAM, KIM-1, CD40, LAP TGF-b1, EGF, Axl, and/or LDH in the second sample profile as compared with a control profile after contacting the subject with a cancer therapeutic agent is indicative of higher susceptibility to treatment with an agent capable of inhibiting or reversing EMT or a chemotherapeutic agent. In some preferred embodiments the control profile is obtained from the same subject prior to treatment with an agent capable of inhibiting or reversing EMT or a chemotherapeutic agent.

Post-Treatment Predictive Biomarkers and Methods

By analysing patient data obtained from clinical trial BGBIL006 (NCT02872259), the authors have discovered that, following treatment of subjects having melanoma with an agent capable of inhibiting or reversing EMT or a chemotherapeutic agent, the levels of certain of the biomarkers described herein increases in the serum of “responder” subjects but not in the serum of “non-responder” subjects. Similarly, following treatment with an agent capable of inhibiting or reversing EMT or a chemotherapeutic agent, the levels of certain of the biomarkers described herein decreases in the serum of “responder” subjects but not in the serum of “non-responder” subjects. Thus, by assessing the activity, expression, or amount of one or more of the biomarkers described herein prior to and following treatment with an agent capable of inhibiting or reversing EMT or a chemotherapeutic agent it is possible to determine or confirm that the subject is susceptible or responsive to treatment with said agent.

Accordingly, in an second aspect of this facet the disclosure provides a method of predicting a cancer-related outcome in a subject having, suspected of having, or diagnosed with melanoma, the method comprising assessing the activity, expression, or amount of one or more of the biomarkers described herein in a subject, or in a sample derived from the subject after the subject has been contacted with an agent capable of inhibiting or reversing EMT or a chemotherapeutic agent. In some embodiments the cancer-related outcome is susceptibility to treatment with an agent capable of inhibiting or reversing EMT or a chemotherapeutic agent.

In some embodiments the method comprises assessing the activity, expression, or amount of one or more of the biomarkers described herein in the subject, or in a sample derived from the subject, to obtain a sample profile of the one or more biomarker; and, making a prediction based on the sample profile of the one or more biomarker. In some embodiments of the method the prediction is made by comparing the sample profile to a control profile. In preferred embodiments the sample profile is obtained after contacting the subject with an agent capable of inhibiting or reversing EMT or a chemotherapeutic agent.

A “control profile” as used herein is a profile of numerical values or numerical ranges corresponding to the activity, expression, or amount of the same biomarkers as for the sample profile, determined in a control subject or population of control subjects.

In preferred embodiments of this aspect of the disclosure the control profile is obtained from the same subject prior to treatment with an agent capable of inhibiting or reversing EMT or a chemotherapeutic agent. In some embodiments the control profile is obtained from a population of control subjects having melanoma. In other embodiments, the control profile is a predetermined profile of biomarker expression, activity, or amount values, for example a profile of “threshold” values or “standard ranges” of values.

Accordingly, in embodiments in which the control profile is obtained from the same subject prior to treatment with an agent capable of inhibiting or reversing EMT or a chemotherapeutic agent, an increase in the activity, expression, or amount of one or more biomarker in the sample profile as compared with a control profile after contacting the subject with an agent capable of inhibiting or reversing EMT or a cancer therapeutic agent may be indicative of susceptibility to treatment with an agent capable of inhibiting or reversing EMT or a chemotherapeutic agent. In such embodiments, a decrease in the activity, expression, or amount of one or more biomarker in the sample profile as compared with a control profile after contacting the subject with a cancer therapeutic agent may be indicative of higher susceptibility to treatment with an agent capable of inhibiting or reversing EMT or a chemotherapeutic agent.

In some such embodiments, an increase in the activity, expression, or amount of one or more biomarker in the sample profile as compared with a control profile after contacting the subject with a cancer therapeutic agent may be indicative of lower susceptibility to treatment with an agent capable of inhibiting or reversing EMT or a chemotherapeutic agent. In some such embodiments, a decrease in the activity, expression, or amount of one or more biomarker in the sample profile as compared with a control profile after contacting the subject with a cancer therapeutic agent may be indicative of lower susceptibility to treatment with an agent capable of inhibiting or reversing EMT or a chemotherapeutic agent.

In some preferred embodiments the one or more biomarker includes at least HB-EGF. In some embodiments the one or more biomarker is HB-EGF. In some embodiments an increase in the activity, expression, or amount of HB-EGF in the sample profile as compared with a control profile after contacting the subject with a cancer therapeutic agent is indicative of higher susceptibility to treatment with an agent capable of inhibiting or reversing EMT or a chemotherapeutic agent.

In some preferred embodiments the one or more biomarker includes at least AFP. In some embodiments the one or more biomarker is AFP. In some embodiments an increase in the activity, expression, or amount of AFP in the sample profile as compared with a control profile after contacting the subject with a cancer therapeutic agent is indicative of higher susceptibility to treatment with an agent capable of inhibiting or reversing EMT or a chemotherapeutic agent.

In some preferred embodiments the one or more biomarker includes at least CgA. In some embodiments the one or more biomarker is CgA. In some embodiments an increase in the activity, expression, or amount of CgA in the sample profile as compared with a control profile after contacting the subject with a cancer therapeutic agent is indicative of higher susceptibility to treatment with an agent capable of inhibiting or reversing EMT or a chemotherapeutic agent.

In some preferred embodiments the one or more biomarker includes at least Nr-CAM. In some embodiments the one or more biomarker is Nr-CAM. In some embodiments an increase in the activity, expression, or amount of Nr-CAM in the sample profile as compared with a control profile after contacting the subject with a cancer therapeutic agent is indicative of higher susceptibility to treatment with an agent capable of inhibiting or reversing EMT or a chemotherapeutic agent.

In some preferred embodiments the one or more biomarker includes at least KIM-1. In some embodiments the one or more biomarker is KIM-1. In some embodiments an increase in the activity, expression, or amount of KIM-1 in the sample profile as compared with a control profile after contacting the subject with a cancer therapeutic agent is indicative of higher susceptibility to treatment with an agent capable of inhibiting or reversing EMT or a chemotherapeutic agent.

In some preferred embodiments the one or more biomarker includes at least CD40. In some embodiments the one or more biomarker is CD40. In some embodiments an increase in the activity, expression, or amount of CD40 in the sample profile as compared with a control profile after contacting the subject with a cancer therapeutic agent is indicative of higher susceptibility to treatment with an agent capable of inhibiting or reversing EMT or a chemotherapeutic agent.

In some preferred embodiments the one or more biomarker includes at least LAP TGF-b1. In some embodiments the one or more biomarker is LAP TGF-b1. In some embodiments an increase in the activity, expression, or amount of LAP TGF-b1 in the sample profile as compared with a control profile after contacting the subject with a cancer therapeutic agent is indicative of higher susceptibility to treatment with an agent capable of inhibiting or reversing EMT or a chemotherapeutic agent.

In some preferred embodiments the one or more biomarker includes at least EGF. In some embodiments the one or more biomarker is EGF. In some embodiments an increase in the activity, expression, or amount of EGF in the sample profile as compared with a control profile after contacting the subject with a cancer therapeutic agent is indicative of higher susceptibility to treatment with an agent capable of inhibiting or reversing EMT or a chemotherapeutic agent.

In some preferred embodiments the one or more biomarker includes at least Axl. In some embodiments the one or more biomarker is Axl. In some embodiments an increase in the activity, expression, or amount of Axl in the sample profile as compared with a control profile after contacting the subject with a cancer therapeutic agent is indicative of higher susceptibility to treatment with an agent capable of inhibiting or reversing EMT or a chemotherapeutic agent.

Subject

A “subject” as referred to herein is any species of animal classified as a mammal, and includes but is not restricted to, domestic and farm animals, primates and humans. In some preferred embodiments the subject is a human of any sex or race. In some embodiments, the human is an adult human.

In some embodiments of this aspect of the disclosure, the subject was selected for treatment with the agent capable of inhibiting or reversing EMT or a chemotherapeutic agent, using a predictive method according to the first aspect of this facet of the disclosure outlined above.

Agents Capable of Inhibiting or Reversing EMT

The present disclosure provides methods for predicting a cancer-related outcome in a subject having, suspected of having, or diagnosed with melanoma. A “cancer-related outcome” is a clinical prediction or prognosis associated with cancer.

The agents capable of inhibiting or reversing EMT for use in this melenoma facet of the disclosure are defined as described above in the AML facet (see identically titled section above on page 14, line 19 to page 17, line 2), except with preferred embodiments as set out below.

In some preferred embodiments the further cancer treatment is a PD-1 inhibitor or PD-L1 inhibitor. In some preferred embodiments the further cancer treatment is a monoclonal antibody such as pembrolizumab, alemtuzumab, bevacizumab, cetuximab, ibritumomab tiuxetan, or rituximab, or is a B-Raf inhibitor such as debrafenib, a MEK inhibitor such as trametinib or a combination therapy of these agents. In some particularly preferred embodiments the further cancer treatment is pembrolizumab. In other particularly preferred embodiments the further cancer treatment is a combination therapy of debrafenib and trametinib.

Assessing Biomarker Activity, Expression, Amount

In some embodiments of the predictive methods described herein, assessing the activity, expression, or amount of one or more biomarker in a subject, or in a sample derived from a subject, comprises: contacting a sample, or an extract from a sample, with at least 1, 2, 3, 4, 5, 6, 7, 8, 9, 10 or more reagents for detecting the activity, expression, or amount of one or more biomarkers, each of which is selective for a particular biomarker; and, detecting and/or quantifying said reagents for detecting.

In some embodiments comprising assessing the activity of LDH, assessing the activity of LDH comprises determining the enzymatic activity of LDH in the subject or sample derived from the subject. Preferably the enzymatic activity is assessed by determining the level or amount of a substrate or product of the enzymatic reaction catalysed by LDH in the subject or sample derived from the subject. In particularly preferred embodiments the enzymatic activity is assessed by determining the level or amount of reduced nicotinamide adenine dinucleotide (NADH) in the subject or sample derived from the subject. In some embodiments this may be determined using a colorimetric assay, for example, the colorimetric assay available from Sigma-Aldrich with catalogue number MAK066. Other suitable assays for assessing activity of LDH are well known to the skilled person. In particularly preferred embodiments the activity of LDH is assessed by determining the enzymatic activity of LDH in blood or serum.

In some preferred embodiments, assessing the expression or amount of the one or more biomarker comprises determining the level of the one or more biomarker protein or mRNA in the subject or sample derived from the subject. Preferably the expression or amount of the one or more biomarker is assessed by determining the level of protein expression. In particularly preferred embodiments the expression or amount of the one or more biomarker is assessed by determining the level of protein expression in serum.

The term “expression” refers to the transcription of a gene's DNA template to produce the corresponding mRNA and translation of this mRNA to produce the corresponding gene product (i.e., a peptide, polypeptide, or protein) as well as the “expression” of a protein in one or more forms that may have been modified post translation.

In some embodiments determining the level of the one or more biomarker protein or mRNA comprises: contacting a sample, or an extract from a sample, with at least 1, 2, 3, 4, 5, 6, 7, 8, 9, 10 or more specific binding members, each of which selectively binds to a respective biomarker protein or mRNA; and, detecting and/or quantifying formation of complex formed by said specific binding member and said biomarker protein or mRNA. In some embodiments the specific binding member may comprise an antibody molecule or a binding fragment thereof.

A range of suitable techniques for determining protein and mRNA expression levels are well known in the art, for example microarray analysis, Western blotting, and PCR techniques such as QPCR. In some embodiments, determining the expression or amount of the one or more biomarker protein or mRNA may comprise use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.

In some embodiments, the expression or amount of the one or more biomarker is determined as an absolute amount of the respective biomarker protein or mRNA in the sample. In such cases, the respective biomarker protein or mRNA may be quantified in the sample and the amount expressed as a measure of the sample, for example the volume, mass, or total protein content of the sample.

In some embodiments, the expression or amount of the one or more biomarker is determined as a relative amount of the respective biomarker protein or mRNA in the sample, wherein the relative amount is determined relative to a reference protein or mRNA in the sample. Determining the relative amount of biomarker protein or mRNA in this way allows for normalisation to, e.g., account for differences in total protein concentration and to remove bias from sample to sample.

Preferably the reference protein or mRNA is one whose expression or amount does not vary significantly between subjects having cancer and subjects not having cancer, in particular melanoma. That is, the reference protein or mRNA is preferably one whose expression or amount is not altered by melanoma.

Accordingly, the reference protein or mRNA may comprise one or more protein or mRNA which is not one of the biomarkers described herein. When expressing the expression or amount of the one or more biomarker as a relative amount, the absolute amount of the respective biomarker protein or mRNA in the sample may be divided by the absolute amount of the reference protein or mRNA in the sample.

Assessing Biomarker Expression at the RNA Level

Gene expression can be detected at the RNA level. RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland). Typical assay formats utilising ribonucleic acid hybridisation include nuclear run-on assays, RT-PCR, RNase protection assays (Melton et al., Nuc. Acids Res. 12:7035), Northern blotting and In Situ hybridization. Gene expression can also be detected by microarray analysis as described below.

Assessing Biomarker Expression at the Protein Level

Preferably biomarker level is assessed by measuring protein expression. Altered gene or protein expression may also be detected by measuring the polypeptides encoded by the gene. This may be achieved by using molecules which bind to the polypeptides encoded by the biomarker gene. Suitable molecules/agents which bind either directly or indirectly to the polypeptides in order to detect the presence of the protein include naturally occurring molecules such as peptides and proteins, for example antibodies, or they may be synthetic molecules.

Antibodies may be derived from commercial sources or through techniques which are familiar to those skilled in the art. In one embodiment, and where altered expression manifests itself through the expression of alteration of post translationally-modified forms of a protein biomarker, antibodies specific for those different forms may be used. For the purposes of this disclosure, the term “antibody”, unless specified to the contrary, includes whole antibodies, or fragments of whole antibodies which retain their binding activity for a target antigen. Such fragments include Fv, F(ab′) and F(ab′)2 fragments, as well as single chain antibodies (scFv). Furthermore, the antibodies and fragments thereof may be humanised antibodies, for example as described in EP239400A. For example: monoclonal and polyclonal antibodies, recombinant antibodies, proteolytic and recombinant fragments of antibodies (Fab, Fv, scFv, diabodies), single-domain antibodies (VHH, sdAb, nanobodies, IgNAR, VNAR), and proteins unrelated to antibodies, which have been engineered to have antibody-like specific binding. Antibodies may be bound to a solid support and/or packaged into kits in a suitable container along with suitable reagents, controls, instructions and the like.

Arrays

Array technology and the various techniques and applications associated with it are well known in the art. Array technology overcomes the disadvantages with traditional methods in molecular biology, which generally work on a “one gene in one experiment” basis, resulting in low throughput and the inability to appreciate the “whole picture” of gene function. In the context of the products and methods of the present disclosure, array technology can be used, for example, in the analysis of the expression of biomarker proteins or mRNA.

In general, any library or group of samples may be arranged in an orderly manner into an array, by spatially separating the members of the library or group. Examples of suitable libraries for arraying include nucleic acid libraries (including DNA, cDNA, oligonucleotide, etc. libraries), peptide, polypeptide and protein libraries, as well as libraries comprising any molecules, such as ligand libraries, among others. Accordingly, where reference is made to a “library” in this document, unless the context dictates otherwise, such reference should be taken to include reference to a library in the form of an array.

Proteins, polypeptides, etc., may also be immobilised in arrays. For example, antibodies have been used in microarray analysis of the proteome using protein chips (Borrebaeck C A, 2000, Immunol Today 21(8):379-82). Polypeptide arrays are reviewed in, for example, MacBeath and Schreiber, 2000, Science, 289(5485):1760-1763.

Sample

Suitable samples include, but are not limited to, tissue samples such as tissue biopsy, blood, urine, buccal scrapes etc., as well as serum, plasma, or tissue culture supernatant samples. In preferred embodiments the expression or amount of the one or more biomarker is assessed by determining the level of protein expression in the sample. In some preferred embodiments the sample is a blood, serum, or plasma sample. In some particularly preferred embodiments the sample is a serum sample.

When assessing the expression or amount of the one or more biomarker proteins or mRNA in serum or plasma samples derived from a subject, samples are removed and subjected to analytical techniques such as flow cytometry, mass cytometry (CyTOF), ELISA, PET, and SELDI-TOF MS. In some embodiments, the method may comprise extracting RNA from said sample and detecting gene expression by QPCR. In some other embodiments, gene expression may be detected by detecting protein products by, for example, Western Blot.

In some embodiments, the level of expression of the one or more biomarker is assessed by determining the copy number of the gene encoding the one or more biomarker. Copy number (i.e. gene duplication events) may be determined using standard techniques known in the art, e.g. using a DNA chip as described in Jiang et al. (Jiang Q, Ho Y Y, Hao L, Nichols Berrios C, Chakravarti A. Copy number variants in candidate genes are genetic modifiers of Hirschsprung disease. PLoS One. 2011; 6(6)).

Is some preferred embodiments of the predictive methods described herein, the method is performed in vitro or ex vivo.

Methods of Selecting a Subject for Treatment

A third aspect of this facet of the present disclosure relates to methods of selecting subjects having, suspected of having, or diagnosed with melanoma for treatment with an agent capable of inhibiting or reversing EMT or a chemotherapeutic agent. In some embodiments the method comprises: identifying subjects susceptible to treatment with an agent capable of inhibiting or reversing EMT or a chemotherapeutic agent using a predictive method defined in accordance with the above described first and second aspects of this facet of the disclosure; and, selecting thus identified subjects for treatment.

In some embodiments this aspect relates to methods of selecting subjects having, suspected of having, or diagnosed with melanoma for continued treatment with an agent capable of inhibiting or reversing EMT or a chemotherapeutic agent. In some embodiments the method comprises: identifying subjects susceptible to treatment with an agent capable of inhibiting or reversing EMT or a chemotherapeutic agent using a predictive method defined in accordance with the above described second aspect of the disclosure; and, selecting thus identified subjects for treatment.

In some embodiments of this aspect of the disclosure, the treatment comprises administering to the subject a therapeutically effective amount of an agent capable of inhibiting or reversing EMT or a chemotherapeutic agent as described herein. Accordingly, in some embodiments the treatment comprises an Axl inhibitor, Akt3 inhibitor, Slfn11 inhibitor, or PHGDH inhibitor. In some preferred embodiments the treatment comprises an Axl inhibitor, for example, BGB324/R428/bemcentinib, cabozantinib, TP-0903, foretinib, merestinib, bosutinib, gilteritinib, crizotinib, amuvatinib, sunitinib, sitravatinib, LDC1267, or UNC2025. Other Axl inhibitors include the anti-Axl antibodies described in WO2015/193428, WO2015/193430, WO2016/097370, and WO2016/166296. In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In some other preferred embodiments, the treatment comprises an Akt3 inhibitor. In some embodiments the Akt3 inhibitor is an Akt3 inhibitor disclosed in WO2016/102672.

In some embodiments of this aspect of the disclosure, the treatment is administered as a single agent. In other embodiments, the treatment is administered in combination with a further cancer treatment. Suitable further cancer treatments are outlined in detail above. In some preferred embodiments the further cancer treatment is a PD-1 inhibitor or PD-L1 inhibitor. In some embodiments the further cancer treatment is a monoclonal antibody such as pembrolizumab, alemtuzumab, bevacizumab, cetuximab, ibritumomab tiuxetan, or rituximab, or is a B-Raf inhibitor such as debrafenib, a MEK inhibitor such as trametinib or a combination therapy of these agents. In some particularly preferred embodiments the further cancer treatment is pembrolizumab. In other particularly preferred embodiments the further cancer treatment is a combination therapy of debrafenib and trametinib.

Kits

A fourth aspect of this facet of the present disclosure relates to diagnostic kits and test devices. In some embodiments the disclosure provides a diagnostic kit comprising 1, 2, 3, 4, 5, 6, 7, 8, 9, 10 or more reagents for detecting the activity, expression, or amount of one or more of the biomarkers described herein, each of which is selective for a particular biomarker. In some embodiments each of the reagents for detecting is selective for a biomarker selected from the group consisting of: HB-EGF, AFP, CgA, Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH.

In other embodiments the disclosure provides a test device comprising 1, 2, 3, 4, 5, 6, 7, 8, 9, 10 or more reagents for detecting the activity, expression, or amount of one or more of the biomarkers described herein, each of which is selective for a particular biomarker. In some embodiments each of the reagents for detecting is selective for a biomarker selected from the group consisting of: HB-EGF, AFP, CgA, Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH.

In some embodiments the diagnostic kit or test device comprises 1, 2, 3, 4, 5, 6, 7, 8, 9, 10 or more specific binding members, each of which selectively binds to one of the biomarkers described herein; and, one or more reagents for detecting said one or more specific binding members, or one or more reagents for detecting and/or quantifying formation of a complex formed by said specific binding member and said biomarker. In some embodiments each specific binding member selectively binds to a biomarker selected from the group consisting of: HB-EGF, AFP, CgA, Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH.

In some embodiments the diagnostic kit or test device comprises a specific binding member which selectively binds to Axl, and at least 1, 2, 3, 4, 5, 6, 7, 8, 9, 10, or more specific binding members, each of which selectively binds to a biomarker selected from the group consisting of: HB-EGF, AFP, CgA, Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, and/or LDH. In some embodiments the diagnostic kit or test device comprises a specific binding member which selectively binds to LDH, and at least 1, 2, 3, 4, 5, 6, 7, 8, 9, 10, or more specific binding members, each of which selectively binds to a biomarker selected from the group consisting of: HB-EGF, AFP, CgA, Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, and/or Axl. In some embodiments the diagnostic kit or test device comprises a specific binding member which selectively binds to HB-EGF, and at least 1, 2, 3, 4, 5, 6, 7, 8, 9, 10, or more specific binding members, each of which selectively binds to a biomarker selected from the group consisting of: AFP, CgA, Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH. In some embodiments the diagnostic kit or test device comprises a specific binding member which selectively binds to AFP, and at least 1, 2, 3, 4, 5, 6, 7, 8, 9, 10, or more specific binding members, each of which selectively binds to a biomarker selected from the group consisting of: HB-EGF, CgA, Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH. In some embodiments the diagnostic kit or test device comprises a specific binding member which selectively binds to CgA, and at least 1, 2, 3, 4, 5, 6, 7, 8, 9, 10, or more specific binding members, each of which selectively binds to a biomarker selected from the group consisting of: HB-EGF, AFP, Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH. In some embodiments the diagnostic kit or test device comprises a specific binding member which selectively binds to Nr-CAM, and at least 1, 2, 3, 4, 5, 6, 7, 8, 9, 10, or more specific binding members, each of which selectively binds to a biomarker selected from the group consisting of: HB-EGF, AFP, CgA, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH. In some embodiments the diagnostic kit or test device comprises a specific binding member which selectively binds to KIM-1, and at least 1, 2, 3, 4, 5, 6, 7, 8, 9, 10, or more specific binding members, each of which selectively binds to a biomarker selected from the group consisting of: HB-EGF, AFP, CgA, Nr-CAM, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH. In some embodiments the diagnostic kit or test device comprises a specific binding member which selectively binds to CD40, and at least 1, 2, 3, 4, 5, 6, 7, 8, 9, 10, or more specific binding members, each of which selectively binds to a biomarker selected from the group consisting of: HB-EGF, AFP, CgA, Nr-CAM, KIM-1, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH. In some embodiments the diagnostic kit or test device comprises a specific binding member which selectively binds to LAP-TGF b1, and at least 1, 2, 3, 4, 5, 6, 7, 8, 9, 10, or more specific binding members, each of which selectively binds to a biomarker selected from the group consisting of: HB-EGF, AFP, CgA, Nr-CAM, KIM-1, CD40, EGF, IL-2 receptor alpha, hCG, TARO, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH. In some embodiments the diagnostic kit or test device comprises a specific binding member which selectively binds to EGF, and at least 1, 2, 3, 4, 5, 6, 7, 8, 9, 10, or more specific binding members, each of which selectively binds to a biomarker selected from the group consisting of: HB-EGF, AFP, CgA, Nr-CAM, KIM-1, CD40, LAP TGF-b1, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH. In some embodiments the diagnostic kit or test device comprises a specific binding member which selectively binds to TATI, and at least 1, 2, 3, 4, 5, 6, 7, 8, 9, 10, or more specific binding members, each of which selectively binds to a biomarker selected from the group consisting of: HB-EGF, AFP, CgA, Nr-CAM, KIM-1, CD40, LAP TGF-b1, EGF, IL-2 receptor alpha, hCG, TARC, CD40-L, VEGFR-3, MMP-7, Leptin, Apo A-I, Periostin, ANG-2, Cystatin-C, Hepsin Apo C-I, IL-1RI, Eotaxin-2, MPO, FRTN, Axl, and/or LDH.

In some embodiments the diagnostic kit or test device comprises a specific binding member which selectively binds to HB-EGF, a specific binding member which selectively binds to AFP, a specific binding member which selectively binds to CgA, and at least 1, 2, 3, 4, 5, 6, 7, 8, 9, 10, or more specific binding members, each of which selectively binds to a biomarker selected from the group consisting of: Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH.

In some embodiments of this aspect of the disclosure the diagnostic kit or test device comprises a plurality of said specific binding members provided in the form of an array on a substrate or conjugated to a plurality of particles, such as beads or microspheres. The particles may be encoded with a detectable label. In some embodiments the specific binding member comprises an antibody molecule or a binding fragment thereof.

In some embodiments of the diagnostic kit or test device detecting formation of a complex formed by a specific binding member and a biomarker is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.

In some embodiments of the diagnostic kit or test device, the kit or test device comprises no more than 15, 20, 25, 30, 40, or 50 reagents for detecting. In some embodiments of the diagnostic kit or test device, the kit or test device comprises no more than 15, 20, 25, 30, 40, or 50 specific binding members.

The present disclosure also provides use of such diagnostic kits and test devices in methods of predicting a cancer-related outcome in a subject. In preferred embodiments the cancer-related outcome is susceptibility to treatment with an agent capable of inhibiting or reversing EMT or a chemotherapeutic agent as described herein.

In some embodiments the agent capable of inhibiting or reversing EMT or chemotherapeutic agent is an Axl inhibitor, Akt3 inhibitor, Slfn11 inhibitor, or PHGDH inhibitor. In some preferred embodiments the agent capable of inhibiting or reversing EMT or chemotherapeutic agent is an Axl inhibitor, for example BGB324/R428/bemcentinib, cabozantinib, TP-0903, foretinib, merestinib, bosutinib, gilteritinib, crizotinib, amuvatinib, sunitinib, sitravatinib, LDC1267, or UNC2025. Other Axl inhibitors include the anti-Axl antibodies described in WO2015/193428, WO2015/193430, WO2016/097370, and WO2016/166296. In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In some embodiments of the diagnostic kit or test device for use in a predictive method, the subject has, is suspected of having, or has been diagnosed with melanoma.

In some embodiments the method may be as defined in accordance with the above described first and second aspects of this facet of the disclosure.

A fifth aspect of this facet of the present disclosure relates to the use of a reagent for detecting the activity, expression, or amount of a biomarker selected from the group consisting of: HB-EGF, AFP, CgA, Nr-CAM, KIM-1, CD40, LAP TGF-b1, EGF, IL-2 receptor alpha, hCG, TARO, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH, in a method of predicting a cancer-related outcome in a subject.

A sixth aspect of this facet of the disclosure relates to the use of 1, 2, 3, 4, 5, 6, 7, 8, 9, 10 or more reagents for detecting the activity, expression, or amount a biomarker, each of which is selective for a particular biomarker selected from the group consisting of: HB-EGF, AFP, CgA, Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH, in a method of manufacture of a diagnostic kit or test device for use in a method of predicting a cancer-related outcome in a subject. In some embodiments of these uses the reagent for detecting comprises a specific binding member selective for a particular biomarker. The specific binding member may comprise an antibody molecule or a binding fragment thereof.

In some embodiments the method may be as defined in accordance with the above described first and second aspects of this facet of the disclosure.

Uses

A seventh aspect of this facet of the present disclosure relates to the use of one or more of: HB-EGF, AFP, CgA, Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH as a biomarker for determining if a subject is susceptible to treatment with an agent capable of inhibiting or reversing EMT or a chemotherapeutic agent.

In some embodiments the use relates to use of at least one of HB-EGF, AFP, CgA, Nr-CAM, KIM-1, CD40, LAP TGF-b1, EGF, TATI, Axl, And LDH; and at least 1, 2, 3, 4, 5, 6, 7, 8, 9, 10, or more of: IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, and/or FRTN, as a biomarker for determining if a subject is susceptible to treatment with an agent capable of inhibiting or reversing EMT or a chemotherapeutic agent. In some embodiments the use relates to use of HB-EGF, and at least 1, 2, 3, 4, 5, 6, 7, 8, 9, 10, or more of: AFP, CgA, Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH, as a biomarker for determining if a subject is susceptible to treatment with an agent capable of inhibiting or reversing EMT or a chemotherapeutic agent. In some embodiments the use relates to use of AFP, and at least 1, 2, 3, 4, 5, 6, 7, 8, 9, 10, or more of: HB-EGF, CgA, Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH, as a biomarker for determining if a subject is susceptible to treatment with an agent capable of inhibiting or reversing EMT or a chemotherapeutic agent. In some embodiments the use relates to use of CgA, and at least 1, 2, 3, 4, 5, 6, 7, 8, 9, 10, or more of: AFP, HB-EGF, Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH, as a biomarker for determining if a subject is susceptible to treatment with an agent capable of inhibiting or reversing EMT or a chemotherapeutic agent. In some embodiments the use relates to use of Nr-CAM, and at least 1, 2, 3, 4, 5, 6, 7, 8, 9, 10, or more of: CgA, AFP, HB-EGF, KIM-1, CD40, LAP TGF-b1, EGF, IL-2 receptor alpha, hCG, TARO, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH, as a biomarker for determining if a subject is susceptible to treatment with an agent capable of inhibiting or reversing EMT or a chemotherapeutic agent. In some embodiments the use relates to use of KIM-1, and at least 1, 2, 3, 4, 5, 6, 7, 8, 9, 10, or more of: CgA, AFP, HB-EGF, Nr-CAM, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH, as a biomarker for determining if a subject is susceptible to treatment with an agent capable of inhibiting or reversing EMT or a chemotherapeutic agent. In some embodiments the use relates to use of CD40, and at least 1, 2, 3, 4, 5, 6, 7, 8, 9, 10, or more of: CgA, AFP, HB-EGF, Nr-CAM, KIM-1, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH, as a biomarker for determining if a subject is susceptible to treatment with an agent capable of inhibiting or reversing EMT or a chemotherapeutic agent. In some embodiments the use relates to use of LAP TGF-b1, and at least 1, 2, 3, 4, 5, 6, 7, 8, 9, 10, or more of: CgA, AFP, HB-EGF, Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH, as a biomarker for determining if a subject is susceptible to treatment with an agent capable of inhibiting or reversing EMT or a chemotherapeutic agent. In some embodiments the use relates to use of EGF, and at least 1, 2, 3, 4, 5, 6, 7, 8, 9, 10, or more of: CgA, AFP, HB-EGF, Nr-CAM, KIM-1, CD40, LAP TGF-b1, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH, as a biomarker for determining if a subject is susceptible to treatment with an agent capable of inhibiting or reversing EMT or a chemotherapeutic agent. In some embodiments the use relates to use of TATI and at least 1, 2, 3, 4, 5, 6, 7, 8, 9, 10, or more of: CgA, AFP, HB-EGF, Nr-CAM, KIM-1, CD40, LAP TGF-b1, EGF, IL-2 receptor alpha, hCG, TARO, CD40-L, VEGFR-3, MMP-7, Leptin, Apo A-I, Periostin, ANG-2, Cystatin-C, Hepsin Apo C-I, IL-1RI, Eotaxin-2, MPO, FRTN, Axl, and/or LDH, as a biomarker for determining if a subject is susceptible to treatment with an agent capable of inhibiting or reversing EMT or a chemotherapeutic agent.

In some embodiments the use is as a biomarker in a method as defined in accordance with the above described first and second aspects of this facet of the disclosure.

Methods of Treating a Subject

A eighth aspect of this facet of the present disclosure relates to methods of treating a subject having, suspected of having, or diagnosed with melanoma. In some embodiments the method comprises: identifying a subject for treatment using a predictive method defined in accordance with the above described first and second aspects of this facet of the disclosure; and, administering to the subject a therapeutically effective amount of an agent capable of inhibiting or reversing EMT or a chemotherapeutic agent.

Accordingly, in some embodiments, the method comprises: (a) obtaining a sample from the subject; (b) determining if the subject is susceptible to treatment with an agent capable of inhibiting or reversing EMT or a chemotherapeutic agent using a predictive method defined in accordance with the above described first and second aspects of this facet of the disclosure; and, (c) administering to the subject a therapeutically effective amount of an agent capable of inhibiting or reversing EMT or a chemotherapeutic agent.

In some embodiments the method comprises administering a therapeutically effective amount of an agent capable of inhibiting or reversing EMT or chemotherapeutic agent to a subject identified as susceptible to said treatment using a predictive method defined in accordance with the above described first and second aspects of this facet of the disclosure.

In other embodiments the method comprises: (a) administering to a subject a therapeutically effective amount of an agent capable of inhibiting or reversing EMT or a chemotherapeutic agent; and, (b) determining if the subject is susceptible to said treatment using a predictive method defined in accordance with the above described second aspect of this facet of the disclosure. In some embodiments the method further comprises: (c) administering to the subject one or more further therapeutically effective amounts of an agent capable of inhibiting or reversing EMT or a chemotherapeutic agent provided that the subject is identified as susceptible to said treatment.

A ninth aspect of this facet of the present disclosure relates to an agent capable of inhibiting or reversing EMT or chemotherapeutic agent for use in a method of treating a subject having, suspected of having, or diagnosed with melanoma. In some embodiments the method of treatment is a method as defined above.

A tenth aspect of this facet of the present disclosure relates to use of an agent capable of inhibiting or reversing EMT or chemotherapeutic agent for use in a method of manufacture of a medicament for use in a method of treating a subject having, suspected of having, or diagnosed with melanoma. In some embodiments the method of treatment is a method as defined above.

In some embodiments of these methods, agents for use, or uses, the agent capable of inhibiting or reversing EMT or chemotherapeutic agent comprises an Axl inhibitor, Akt3 inhibitor, Slfn11 inhibitor, or PHGDH inhibitor. In some preferred embodiments the agent capable of inhibiting or reversing EMT or chemotherapeutic agent is an Axl inhibitor, for example BGB324/R428/bemcentinib, cabozantinib, TP-0903, foretinib, merestinib, bosutinib, gilteritinib, crizotinib, amuvatinib, sunitinib, sitravatinib, LDC1267, or UNC2025. Other Axl inhibitors include the anti-Axl antibodies described in WO2015/193428, WO2015/193430, WO2016/097370, and WO2016/166296. In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In some other preferred embodiments, the treatment comprises an Akt3 inhibitor. In some embodiments the Akt3 inhibitor is an Akt3 inhibitor disclosed in WO2016/102672.

In some embodiments of these methods, agents for use, or uses, the treatment is administered as a single agent. In other embodiments, the treatment is administered in combination with a further cancer treatment. Suitable further cancer treatments are outlined in detail above. In some preferred embodiments the further cancer treatment is a PD-1 inhibitor or PD-L1 inhibitor. In some embodiments the further cancer treatment is a monoclonal antibody such as pembrolizumab, alemtuzumab, bevacizumab, cetuximab, ibritumomab tiuxetan, or rituximab, or is a B-Raf inhibitor such as debrafenib, a MEK inhibitor such as trametinib or a combination therapy of these agents. In some particularly preferred embodiments the further cancer treatment is pembrolizumab. In other particularly preferred embodiments the further cancer treatment is a combination therapy of debrafenib and trametinib.

Statements of Disclosure

The following numbered statements relate to aspects of this facet of the present disclosure, and form part of the description:
101. A method of predicting a cancer-related outcome in a subject, the method comprising assessing the activity, expression, or amount of one or more biomarker in the subject, or in a sample derived from the subject;
   wherein the one or more biomarker is selected from the group consisting of: HB-EGF, AFP, CgA, Nr-CAM, KIM-1, CD40, LAP TGF-b1, EGF, IL-2 receptor alpha, hCG, TARO, CD40-L, VEGFR-3, TATI, MMP-7, Leptin, Apo A-I, Periostin, ANG-2, Cystatin-C, Hepsin Apo C-I, IL-1 RI, Eotaxin-2, MPO, FRTN, Axl, and/or LDH; and
   wherein the subject has, is suspected of having, or has been diagnosed with melanoma.
102. A method according to statement 101, wherein the method comprises:
   assessing the activity, expression, or amount of one or more biomarker in the subject, or in a sample derived from the subject, to obtain a sample profile of the one or more biomarker; and
   making a prediction based on the sample profile of the one or more biomarker.
103. A method according to statement 102, wherein the prediction is made by comparing the sample profile to a control profile.
104. A method according to statement 103, wherein the sample profile is obtained before the subject is contacted with or administered an agent capable of inhibiting or reversing EMT or a chemotherapeutic agent.
105. A method according to statement 104, wherein the control profile is:
   (i) obtained from a population of control subjects having melanoma; or
   (ii) obtained from a control subject or population of control subjects having melanoma and previously found to lack susceptibility to treatment with an agent capable of inhibiting or reversing EMT or a chemotherapeutic agent (that is, non-responder subjects);
   (iii) a predetermined profile of biomarker expression, activity, or amount values, for example a profile of “average, median, or mean” values or “standard ranges” of values, obtained from a control subject or population of control subjects having melanoma and previously found to lack susceptibility to treatment with an agent capable of inhibiting or reversing EMT or a chemotherapeutic agent (that is, non-responder subjects);
   (iv) obtained from a control sample having a known “average, median, or mean” value of biomarkers indicative of a control subject or population of control subjects having melanoma and previously found to lack susceptibility to treatment with an agent capable of inhibiting or reversing EMT or a chemotherapeutic agent (that is, non-responder subjects);
   (v) a predetermined profile of biomarker expression, activity, or amount “threshold” values obtained from a control subject or population of control subjects having melanoma and previously found to be susceptible to treatment with an agent capable of inhibiting or reversing EMT or a chemotherapeutic agent (that is, responder subjects); or
   (vi) obtained from a control sample having a known “threshold” value of biomarkers indicative of a control subject or population of control subjects having melanoma and previously found to be susceptible to treatment with an agent capable of inhibiting or reversing EMT or a chemotherapeutic agent (that is, responder subjects).
106. A method according to statement 105, wherein a higher activity, expression, or amount of one or more biomarker in the sample profile as compared with the control profile is indicative of susceptibility to treatment with an agent capable of inhibiting or reversing EMT or a chemotherapeutic agent.
107. A method according to statement 105, wherein a lower activity, expression, or amount of one or more biomarker in the sample profile as compared with the control profile is indicative of susceptibility to treatment with an agent capable of inhibiting or reversing EMT or a chemotherapeutic agent.
108. A method according to any one of statements 105-107, wherein the one or more biomarker includes at least one of: HB-EGF, CgA, Nr-CAM, KIM-1, CD40, LAP TGF-b1, EGF, IL-2 receptor alpha, TARC, CD40-L, VEGFR-3, TATI, MMP-7, Leptin, Apo A-I, ANG-2, Cystatin-C, Hepsin, Apo C-I, IL-1RI, Axl, and/or LDH,
   and wherein a higher activity, expression, or amount of one or more of said biomarkers in the sample profile as compared with the control profile is indicative of susceptibility to treatment with an agent capable of inhibiting or reversing EMT or a chemotherapeutic agent.
109. A method according to any one of statements 105-108, wherein the one or more biomarker includes at least one of: AFP, Periostin, hCG, Eotaxin-2, MPO, and/or FRTN,
   and wherein a lower activity, expression, or amount of one or more of said biomarkers in the sample profile as compared with the control profile is indicative of susceptibility to treatment with an agent capable of inhibiting or reversing EMT or a chemotherapeutic agent.
110. A method according to any one of statements 105-109, wherein:
   a lower activity, expression, or amount of AFP; and/or
   a lower activity, expression, or amount of Periostin; and/or
   a lower activity, expression, or amount of hCG; and/or
   a lower activity, expression, or amount of Eotaxin-2; and/or
   a lower activity, expression, or amount of MPO; and/or
   a lower activity, expression, or amount of FRTN; and/or
   a higher activity, expression, or amount of CgA; and/or
   a higher activity, expression, or amount of HB-EGF; and/or
   a higher activity, expression, or amount of Nr-CAM; and/or
   a higher activity, expression, or amount of KIM-1; and/or
   a higher activity, expression, or amount of CD40; and/or
   a higher activity, expression, or amount of LAP TGF-b1; and/or
   a higher activity, expression, or amount of EGF; and/or
   a higher activity, expression, or amount of IL-2ra; and/or
   a higher activity, expression, or amount of TARO; and/or
   a higher activity, expression, or amount of CD40-L; and/or
   a higher activity, expression, or amount of VEGFR-3; and/or
   a higher activity, expression, or amount of TATI; and/or
   a higher activity, expression, or amount of MMP-7; and/or
   a higher activity, expression, or amount of Leptin; and/or
   a higher activity, expression, or amount of Apo A-I; and/or
   a higher activity, expression, or amount of ANG-2; and/or
   a higher activity, expression, or amount of Cystatin-C; and/or
   a higher activity, expression, or amount of Hepsin; and/or
   a higher activity, expression, or amount of Apo C-I; and/or
   a higher activity, expression, or amount of IL-1RI; and/or
   a higher activity, expression, or amount of Axl; and/or
   a higher activity, expression, or amount of LDH;
   in the sample profile as compared with the control profile is indicative of susceptibility to treatment with an agent capable of inhibiting or reversing EMT or a chemotherapeutic agent.
111. A method according to any one of statements 101-110, wherein the one or more biomarker comprises HB-EGF,
   optionally wherein the one or more biomarker comprises at least 1, 2, 3, 4, 5, 6, 7, 8, 9, 10, or more further biomarkers selected from: HB-EGF, AFP, CgA, Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH.
112. A method according to any one of statements 101-110, wherein the one or more biomarker comprises AFP
   optionally wherein the one or more biomarker comprises at least 1, 2, 3, 4, 5, 6, 7, 8, 9, 10, or more further biomarkers selected from: HB-EGF, CgA, Nr-CAM, KIM-1, CD40, LAP TGF-b1, EGF, IL-2 receptor alpha, hCG, TARO, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH.
113. A method according to any one of statements 101-110, wherein the one or more biomarker comprises CgA,
   optionally wherein the one or more biomarker comprises at least 1, 2, 3, 4, 5, 6, 7, 8, 9, 10, or more further biomarkers selected from: HB-EGF, AFP, Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH.
114. A method according to any one of statements 101-110, wherein the one or more biomarker comprises Nr-CAM,
   optionally wherein the one or more biomarker comprises at least 1, 2, 3, 4, 5, 6, 7, 8, 9, 10, or more further biomarkers selected from: HB-EGF, AFP, CgA, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH.
115. A method according to any one of statements 101-110, wherein the one or more biomarker comprises KIM-1,
   optionally wherein the one or more biomarker comprises at least 1, 2, 3, 4, 5, 6, 7, 8, 9, 10, or more further biomarkers selected from: HB-EGF, AFP, CgA, Nr-CAM, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH.
116. A method according to any one of statements 101-110, wherein the one or more biomarker comprises CD40,
   optionally wherein the one or more biomarker comprises at least 1, 2, 3, 4, 5, 6, 7, 8, 9, 10, or more further biomarkers selected from: HB-EGF, AFP, CgA, Nr-CAM, KIM-1, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH.
117. A method according to any one of statements 101-110, wherein the one or more biomarker comprises LAP TGF-b1,
   optionally wherein the one or more biomarker comprises at least 1, 2, 3, 4, 5, 6, 7, 8, 9, 10, or more further biomarkers selected from: HB-EGF, AFP, CgA, Nr-CAM, KIM-1, CD40, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH.
118. A method according to any one of statements 101-110, wherein the one or more biomarker comprises EGF,
   optionally wherein the one or more biomarker comprises at least 1, 2, 3, 4, 5, 6, 7, 8, 9, 10, or more further biomarkers selected from: HB-EGF, AFP, CgA, Nr-CAM, KIM-1, CD40, LAP TGF-b1, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH.
119. A method according to any one of statements 101-110, wherein the one or more biomarker comprises IL-2ra,
   optionally wherein the one or more biomarker comprises at least 1, 2, 3, 4, 5, 6, 7, 8, 9, 10, or more further biomarkers selected from: HB-EGF, AFP, CgA, Nr-CAM, KIM-1, CD40, LAP TGF-b1, EGF, hCG, TARO, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH.
120. A method according to any one of statements 101-110, wherein the one or more biomarker comprises hCG,
   optionally wherein the one or more biomarker comprises at least 1, 2, 3, 4, 5, 6, 7, 8, 9, 10, or more further biomarkers selected from: HB-EGF, AFP, CgA, Nr-CAM, KIM-1, CD40, LAP TGF-b1, EGF, IL-2 receptor alpha, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH.
121. A method according to any one of statements 101-110, wherein the one or more biomarker comprises TARC,
   optionally wherein the one or more biomarker comprises at least 1, 2, 3, 4, 5, 6, 7, 8, 9, 10, or more further biomarkers selected from: HB-EGF, AFP, CgA, Nr-CAM, KIM-1, CD40, LAP TGF-b1, EGF, IL-2 receptor alpha, hCG, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH.
122. A method according to any one of statements 101-110, wherein the one or more biomarker comprises CD40-L,
   optionally wherein the one or more biomarker comprises at least 1, 2, 3, 4, 5, 6, 7, 8, 9, 10, or more further biomarkers selected from: HB-EGF, AFP, CgA, Nr-CAM, KIM-1, CD40, LAP TGF-b1, EGF, IL-2 receptor alpha, hCG, TARC, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH.
123. A method according to any one of statements 101-110, wherein the one or more biomarker comprises VEGFR-3,
   optionally wherein the one or more biomarker comprises at least 1, 2, 3, 4, 5, 6, 7, 8, 9, 10, or more further biomarkers selected from: HB-EGF, AFP, CgA, Nr-CAM, KIM-1, CD40, LAP TGF-b1, EGF, IL-2 receptor alpha, hCG, TARC, CD40-L, TATI, MMP-7, Leptin, Apo A-I, Periostin, ANG-2, Cystatin-C, Hepsin Apo C-I, IL-1RI, Eotaxin-2, MPO, FRTN, Axl, and/or LDH.
124. A method according to any one of statements 101-110, wherein the one or more biomarker comprises TATI,
   optionally wherein the one or more biomarker comprises at least 1, 2, 3, 4, 5, 6, 7, 8, 9, 10, or more further biomarkers selected from: HB-EGF, AFP, CgA, Nr-CAM, KIM-1, CD40, LAP TGF-b1, EGF, IL-2 receptor alpha, hCG, TARC, CD40-L, VEGFR-3, MMP-7, Leptin, Apo A-I, Periostin, ANG-2, Cystatin-C, Hepsin Apo C-I, IL-1RI, Eotaxin-2, MPO, FRTN, Axl, and/or LDH.
125. A method according to any one of statements 101-110, wherein the one or more biomarker comprises MMP-7,
   optionally wherein the one or more biomarker comprises at least 1, 2, 3, 4, 5, 6, 7, 8, 9, 10, or more further biomarkers selected from: HB-EGF, AFP, CgA, Nr-CAM, KIM-1, CD40, LAP TGF-b1, EGF, IL-2 receptor alpha, hCG, TARC, CD40-L, VEGFR-3, TATI, Leptin, Apo A-I, Periostin, ANG-2, Cystatin-C, Hepsin Apo C-I, IL-1RI, Eotaxin-2, MPO, FRTN, Axl, and/or LDH.
126. A method according to any one of statements 101-110, wherein the one or more biomarker comprises Leptin,
   optionally wherein the one or more biomarker comprises at least 1, 2, 3, 4, 5, 6, 7, 8, 9, 10, or more further biomarkers selected from: HB-EGF, AFP, CgA, Nr-CAM, KIM-1, CD40, LAP TGF-b1, EGF, IL-2 receptor alpha, hCG, TARC, CD40-L, VEGFR-3, TATI, MMP-7, Apo A-I, Periostin, ANG-2, Cystatin-C, Hepsin Apo C-I, IL-1RI, Eotaxin-2, MPO, FRTN, Axl, and/or LDH.
127. A method according to any one of statements 101-110, wherein the one or more biomarker comprises Apo A-I,
   optionally wherein the one or more biomarker comprises at least 1, 2, 3, 4, 5, 6, 7, 8, 9, 10, or more further biomarkers selected from: HB-EGF, AFP, CgA, Nr-CAM, KIM-1, CD40, LAP TGF-b1, EGF, IL-2 receptor alpha, hCG, TARO, CD40-L, VEGFR-3, TATI, MMP-7, Leptin, Periostin, ANG-2, Cystatin-C, Hepsin Apo C-I, IL-1RI, Eotaxin-2, MPO, FRTN, Axl, and/or LDH.
128. A method according to any one of statements 101-110, wherein the one or more biomarker comprises Periostin,
   optionally wherein the one or more biomarker comprises at least 1, 2, 3, 4, 5, 6, 7, 8, 9, 10, or more further biomarkers selected from: HB-EGF, AFP, CgA, Nr-CAM, KIM-1, CD40, LAP TGF-b1, EGF, IL-2 receptor alpha, hCG, TARC, CD40-L, VEGFR-3, TATI, MMP-7, Leptin, Apo A-I, ANG-2, Cystatin-C, Hepsin Apo C-I, IL-1RI, Eotaxin-2, MPO, FRTN, Axl, and/or LDH.
129. A method according to any one of statements 101-110, wherein the one or more biomarker comprises ANG-2,
   optionally wherein the one or more biomarker comprises at least 1, 2, 3, 4, 5, 6, 7, 8, 9, 10, or more further biomarkers selected from: HB-EGF, AFP, CgA, Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, Cystatin-C, Hepsin Apo C-I, IL-1RI, Eotaxin-2, MPO, FRTN, Axl, and/or LDH.
130. A method according to any one of statements 101-110, wherein the one or more biomarker comprises Cystain-C,
   optionally wherein the one or more biomarker comprises at least 1, 2, 3, 4, 5, 6, 7, 8, 9, 10, or more further biomarkers selected from: HB-EGF, AFP, CgA, Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Hepsin Apo C-I, IL-1RI, Eotaxin-2, MPO, FRTN, Axl, and/or LDH.
131. A method according to any one of statements 101-110, wherein the one or more biomarker comprises Hepsin,
   optionally wherein the one or more biomarker comprises at least 1, 2, 3, 4, 5, 6, 7, 8, 9, 10, or more further biomarkers selected from: HB-EGF, AFP, CgA, Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Apo C-I, IL-1RI, Eotaxin-2, MPO, FRTN, Axl, and/or LDH.
132. A method according to any one of statements 101-110, wherein the one or more biomarker comprises Apo C-I,
   optionally wherein the one or more biomarker comprises at least 1, 2, 3, 4, 5, 6, 7, 8, 9, 10, or more further biomarkers selected from: HB-EGF, AFP, CgA, Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin, IL-1RI, Eotaxin-2, MPO, FRTN, Axl, and/or LDH.
133. A method according to any one of statements 101-110, wherein the one or more biomarker comprises IL-1RI,
   optionally wherein the one or more biomarker comprises at least 1, 2, 3, 4, 5, 6, 7, 8, 9, 10, or more further biomarkers selected from: HB-EGF, AFP, CgA, Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, Eotaxin-2, MPO, FRTN, Axl, and/or LDH.
134. A method according to any one of statements 101-110, wherein the one or more biomarker comprises HB-EGF,
   optionally wherein the one or more biomarker comprises at least 1, 2, 3, 4, 5, 6, 7, 8, 9, 10, or more further biomarkers selected from: HB-EGF, AFP, CgA, Nr-CAM, KIM-1, CD40, LAP TGF-b1, EGF, IL-2 receptor alpha, hCG, TARO, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH.
135. A method according to any one of statements 101-110, wherein the one or more biomarker comprises Eotaxin-2,
   optionally wherein the one or more biomarker comprises at least 1, 2, 3, 4, 5, 6, 7, 8, 9, 10, or more further biomarkers selected from: HB-EGF, AFP, CgA, Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, MPO, FRTN, Axl, and/or LDH.
136. A method according to any one of statements 101-110, wherein the one or more biomarker comprises MPO,
   optionally wherein the one or more biomarker comprises at least 1, 2, 3, 4, 5, 6, 7, 8, 9, 10, or more further biomarkers selected from: HB-EGF, AFP, CgA, Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, FRTN, Axl, and/or LDH.
137. A method according to any one of statements 101-110, wherein the one or more biomarker comprises FRTN,
   optionally wherein the one or more biomarker comprises at least 1, 2, 3, 4, 5, 6, 7, 8, 9, 10, or more further biomarkers selected from: HB-EGF, AFP, CgA, Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, Axl, and/or LDH.
138. A method according to any one of statements 101-110, wherein the one or more biomarker comprises Axl,
   optionally wherein the one or more biomarker comprises at least 1, 2, 3, 4, 5, 6, 7, 8, 9, 10, or more further biomarkers selected from: HB-EGF, AFP, CgA, Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, and/or LDH.
139. A method according to any one of statements 101-110, wherein the one or more biomarker comprises LDH,
   optionally wherein the one or more biomarker comprises at least 1, 2, 3, 4, 5, 6, 7, 8, 9, 10, or more further biomarkers selected from: HB-EGF, AFP, CgA, Nr-CAM, KIM-1, CD40, LAP TGF-b1, EGF, TATI, IL-2 receptor alpha, hCG, TARC, CD40-L, VEGFR-3, MMP-7, Leptin, Apo A-I, Periostin, ANG-2, Cystatin-C, Hepsin Apo C-I, IL-1RI, Eotaxin-2, MPO, FRTN, and/or Axl.
140. A method according to any one of statements 111-139, wherein the one or more biomarker further comprises AFP.
141. A method according to any one of statements 111-140, wherein the one or more biomarker further comprises CgA.
142. A method according to any one of statements 111-141, wherein the one or more biomarker further comprises Nr-CAM.
143. A method according to any one of statements 111-142, wherein the one or more biomarker further comprises KIM-1.
144. A method according to any one of statements 111-143, wherein the one or more biomarker further comprises CD40.
145. A method according to any one of statements 111-144, wherein the one or more biomarker further comprises LAP-TGF-b1.
146. A method according to any one of statements 111-145, wherein the one or more biomarker further comprises EGF.
147. A method according to any one of statements 111-146, wherein the one or more biomarker further comprises TATI.
148. A method according to any one of statements 111-147, wherein the one or more biomarker further comprises Axl.
149. A method according to any one of statements 111-148, wherein the one or more biomarker further comprises LDH.
150. A method according to any one of statements 111-149, wherein the one or more biomarker further comprises IL-2ra.
151. A method according to any one of statements 111-150, wherein the one or more biomarker further comprises hCG.
152. A method according to any one of statements 111-151, wherein the one or more biomarker further comprises TARO.
153. A method according to any one of statements 111-152, wherein the one or more biomarker further comprises CD40-L.
154. A method according to any one of statements 111-153, wherein the one or more biomarker further comprises VEGFR-3.
155. A method according to any one of statements 111-154, wherein the one or more biomarker further comprises MMP-7.
156. A method according to any one of statements 111-155, wherein the one or more biomarker further comprises Leptin.
157. A method according to any one of statements 111-156, wherein the one or more biomarker further comprises Apo A-I.
158. A method according to any one of statements 111-157, wherein the one or more biomarker further comprises Periostin.
159. A method according to any one of statements 111-158, wherein the one or more biomarker further comprises ANG-2.
160. A method according to any one of statements 111-159, wherein the one or more biomarker further comprises Cystatin-C.
161. A method according to any one of statements 111-160, wherein the one or more biomarker further comprises Hepsin.
162. A method according to any one of statements 111-161, wherein the one or more biomarker further comprises Apo C-I.
163. A method according to any one of statements 111-162, wherein the one or more biomarker further comprises IL-1RI.
164. A method according to any one of statements 111-163, wherein the one or more biomarker further comprises Eotaxin-2.
165. A method according to any one of statements 111-164, wherein the one or more biomarker further comprises MPO.
166. A method according to any one of statements 111-165, wherein the one or more biomarker further comprises FRTN.
167. A method according to any one of statements 101-166, wherein:
   the Axl biomarker comprises an amino acid sequence having at least 70%, 80%, 90%, 95%, 99% or 100% sequence identity with the full-length of the amino acid sequence disclosed at UniProt accession no: P30530-1 or P30530-2 (entry version 196) or a fragment thereof;
   the Alpha-Fetoprotein (AFP) biomarker comprises an amino acid sequence having at least 70%, 80%, 90%, 95%, 99% or 100% sequence identity with the full-length of the amino acid sequence disclosed at UniProt accession no: P02771-1 (entry version 192) or a fragment thereof;
   the Heparin-Binding EGF-Like Growth Factor (HB-EGF) biomarker comprises an amino acid sequence having at least 70%, 80%, 90%, 95%, 99% or 100% sequence identity with the full-length of the amino acid sequence disclosed at UniProt accession no: Q99075 (entry version 171) or a fragment thereof;
   the Neuronal Cell Adhesion Molecule (Nr-CAM) biomarker comprises an amino acid sequence having at least 70%, 80%, 90%, 95%, 99% or 100% sequence identity with the full-length of the amino acid sequence disclosed at UniProt accession no: Q92823-1, Q92823-2, Q92823-3, Q92823-4, Q92823-5, or Q92823-6 (entry version 172) or a fragment thereof;
   the Kidney Injury Molecule-1 (KIM-1) biomarker comprises an amino acid sequence having at least 70%, 80%, 90%, 95%, 99% or 100% sequence identity with the full-length of the amino acid sequence disclosed at UniProt accession no: Q96D42-1 (entry version 141) or a fragment thereof;
   the CD 40 antigen (CD40) comprises an amino acid sequence having at least 70%, 80%, 90%, 95%, 99% or 100% sequence identity with the full-length of the amino acid sequence disclosed at UniProt accession no: P25942-1 or P25942-2 (entry version 221) or a fragment thereof;
   the Chromogranin-A (CgA) biomarker comprises an amino acid sequence having at least 70%, 80%, 90%, 95%, 99% or 100% sequence identity with the full-length of the amino acid sequence disclosed at UniProt accession no: P10645-1 (entry version 200) or a fragment thereof;
   the Interleukin-2 receptor alpha (IL-2 receptor alpha) biomarker comprises an amino acid sequence having at least 70%, 80%, 90%, 95%, 99% or 100% sequence identity with the full-length of the amino acid sequence disclosed at UniProt accession no: P01589-1 (entry version 200) or a fragment thereof;
   the Human Chorionic Gonadotropin beta (hCG) comprises an amino acid sequence having at least 70%, 80%, 90%, 95%, 99% or 100% sequence identity with the full-length of the amino acid sequence disclosed at UniProt accession no: PODN86-1 or PODN86-2 (entry version 26) or a fragment thereof;
   the Latency-Associated Peptide of Transforming Growth Factor beta 1 (LAP TGF-b1) biomarker comprises an amino acid sequence having at least 70%, 80%, 90%, 95%, 99% or 100% sequence identity with the full-length of the amino acid sequence disclosed at UniProt accession no: P01137 (entry version 235) or a fragment thereof;
   the Thymus and activation-regulated chemokine (TARO) biomarker comprises an amino acid sequence having at least 70%, 80%, 90%, 95%, 99% or 100% sequence identity with the full-length of the amino acid sequence disclosed at UniProt accession no: Q92583-1 (entry version 163) or a fragment thereof;
   the CD40 Ligand (CD40-L) biomarker comprises an amino acid sequence having at least 70%, 80%, 90%, 95%, 99% or 100% sequence identity with the full-length of the amino acid sequence disclosed at UniProt accession no: P29965 (entry version 199) or a fragment thereof;
   the Epidermal Growth Factor (EGF) biomarker comprises an amino acid sequence having at least 70%, 80%, 90%, 95%, 99% or 100% sequence identity with the full-length of the amino acid sequence disclosed at UniProt accession no: P01133-1, P01133-2, or P01133-3 (entry version 204) or a fragment thereof;
   the Vascular endothelial growth factor receptor 3 (VEGFR-3) biomarker comprises an amino acid sequence having at least 70%, 80%, 90%, 95%, 99% or 100% sequence identity with the full-length of the amino acid sequence disclosed at UniProt accession no: P35916-1, P35916-2, or P35916-3 (entry version 206) or a fragment thereof;
   the Pancreatic secretory trypsin inhibitor (TATI) biomarker comprises an amino acid sequence having at least 70%, 80%, 90%, 95%, 99% or 100% sequence identity with the full-length of the amino acid sequence disclosed at UniProt accession no: P00995-1 (entry version 193) or a fragment thereof;
   the Matrix Metalloproteinase-7 (MMP-7) biomarker comprises an amino acid sequence having at least 70%, 80%, 90%, 95%, 99% or 100% sequence identity with the full-length of the amino acid sequence disclosed at UniProt accession no: P09237-1 (entry version 197) or a fragment thereof;
   the Leptin biomarker comprises an amino acid sequence having at least 70%, 80%, 90%, 95%, 99% or 100% sequence identity with the full-length of the amino acid sequence disclosed at UniProt accession no: P41159 (entry version 176) or a fragment thereof;
   the Apolipoprotein A-I (Apo A-I) biomarker comprises an amino acid sequence having at least 70%, 80%, 90%, 95%, 99% or 100% sequence identity with the full-length of the amino acid sequence disclosed at UniProt accession no: P02647-1 (entry version 241) or a fragment thereof;
   the Periostin biomarker comprises an amino acid sequence having at least 70%, 80%, 90%, 95%, 99% or 100% sequence identity with the full-length of the amino acid sequence disclosed at UniProt accession no: Q15063-1, Q15063-2, Q15063-3, Q15063-4, Q15063-5, Q15063-6, Q15063-7 (entry version 160) or a fragment thereof;
   the Angiopoietin-2 (ANG-2) biomarker comprises an amino acid sequence having at least 70%, 80%, 90%, 95%, 99% or 100% sequence identity with the full-length of the amino acid sequence disclosed at UniProt accession no: 015123-1, 015123-2, or 015123-3 (entry version 163) or a fragment thereof;
   the Cystatin-C biomarker comprises an amino acid sequence having at least 70%, 80%, 90%, 95%, 99% or 100% sequence identity with the full-length of the amino acid sequence disclosed at UniProt accession no: P01034-1 (entry version 213) or a fragment thereof;
   the Hepsin biomarker comprises an amino acid sequence having at least 70%, 80%, 90%, 95%, 99% or 100% sequence identity with the full-length of the amino acid sequence disclosed at UniProt accession no: P05981-1 (entry version 183) or a fragment thereof;
   the Apolipoprotein C-I (Apo C-I) biomarker comprises an amino acid sequence having at least 70%, 80%, 90%, 95%, 99% or 100% sequence identity with the full-length of the amino acid sequence disclosed at UniProt accession no: P02654 (entry version 175) or a fragment thereof;
   the Interleukin-1 receptor type 1 (IL-1RI) biomarker comprises an amino acid sequence having at least 70%, 80%, 90%, 95%, 99% or 100% sequence identity with the full-length of the amino acid sequence disclosed at UniProt accession no: P14778-1 (entry version 215) or a fragment thereof;
   the Eotaxin-2 biomarker comprises an amino acid sequence having at least 70%, 80%, 90%, 95%, 99% or 100% sequence identity with the full-length of the amino acid sequence disclosed at UniProt accession no: 000175-1 (entry version 162) or a fragment thereof;
   the Myeloperoxidase (MPO) biomarker comprises an amino acid sequence having at least 70%, 80%, 90%, 95%, 99% or 100% sequence identity with the full-length of the amino acid sequence disclosed at UniProt accession no: P05164-1, P05164-2, or P05164-3 (entry version 217) or a fragment thereof;
   the Ferritin (FRTN) biomarker comprises an amino acid sequence having at least 70%, 80%, 90%, 95%, 99% or 100% sequence identity with the full-length of the amino acid sequence disclosed at UniProt accession no: P02794 (entry version 213) or a fragment thereof;
   the Lactate Dehydrogenase (LDH) biomarker comprises an amino acid sequence having at least 70%, 80%, 90%, 95%, 99% or 100% sequence identity with the full-length of the amino acid sequence disclosed at UniProt accession no: P00338-1, P00338-2, P00338-3, P00338-4, or P00338-5 (entry version 224) or a fragment thereof; and/or
   the Lactate Dehydrogenase (LDH) biomarker comprises an amino acid sequence having at least 70%, 80%, 90%, 95%, 99% or 100% sequence identity with the full-length of the amino acid sequence disclosed at UniProt accession no: P07195-1 (entry version 218) or a fragment thereof.
   or a nucleic acid (either DNA or RNA) having at least 70%, 80%, 90%, 95%, 99% or 100% sequence identity with the full-length nucleic acid sequence encoding said amino acid sequences.
168. A method according to any one of statement 101-167, wherein the method comprises determining the amount of at least 2, 3, 4, 5, 6, 7, 8, 9, 10, 11, 12, 13, 14, 15, 20 of said biomarkers in the subject, or sample derived from the subject.
169. A method according to any one of statements 104-168, wherein assessing the activity, expression, or amount of one or more biomarker in the subject, or in a sample derived from the subject, comprises:
   contacting a sample, or an extract from the sample, with at least 1, 2, 3, 4, 5, 6, 7, 8, 9, 10 or more reagents for detecting the activity, expression, or amount of one or more biomarkers, each of which is selective for a particular biomarker; and
   detecting and/or quantifying said reagents for detecting.
170. A method according to statement 169, wherein assessing the expression or amount of one or more biomarker in the subject, or in a sample derived from the subject, comprises:
   contacting a sample, or an extract from the sample, with at least 1, 2, 3, 4, 5, 6, 7, 8, 9, 10 or more specific binding members, each of which selectively binds to a respective biomarker protein or mRNA; and
   detecting and/or quantifying formation of complex formed by said specific binding member and said biomarker protein or mRNA.
171. A method according to statement 170 wherein the specific binding member comprises an antibody molecule or binding fragment thereof.
172. A method according to statement 170 or 171, wherein detecting formation of a complex formed by the specific binding member and the biomarker protein or mRNA is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.
173. A method according to any one of statements 170-172 wherein the expression or amount of the one or more biomarker is determined as the absolute amount of the respective biomarker protein or mRNA in the sample.
174. A method according to any one of statements 170-172 wherein the expression or amount of the one or more biomarker is determined as a relative amount of the respective biomarker protein or mRNA in the sample,
   optionally wherein the relative amount is determined relative to a reference protein or mRNA in the sample, the expression of which is not altered by melanoma.
175. A method according to any one of statements 101-174, wherein the cancer-related outcome is susceptibility to treatment with an agent capable of inhibiting or reversing EMT or a chemotherapeutic agent.
176. A method according to statement 174, wherein the agent capable of inhibiting or reversing EMT or chemotherapeutic agent is an Axl inhibitor, Akt3 inhibitor, Slfn11 inhibitor, or PHGDH inhibitor.
177. A method according to statement 176, wherein the agent capable of inhibiting or reversing EMT or chemotherapeutic agent is an Axl inhibitor.
178. A method according to statement 177, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
179. A method according to statement 178, wherein the Axl inhibitor is BGB324/R428/bemcentinib.
180. A method according to statement 178, wherein the Axl inhibitor is an anti-Axl antibody described in WO2015/193428, WO2015/193430, WO2016/097370, or WO2016/166296.
181. A method according to statement 176, wherein the agent capable of inhibiting or reversing EMT or chemotherapeutic agent is an Akt3 inhibitor.
182. A method according to statement 181, wherein the Akt3 inhibitor is an Akt3 inhibitor disclosed in WO2016/102672.
183. A method according to any one of statements 175-182, wherein the agent capable of inhibiting or reversing EMT is administered as a single agent.
184. A method according to any one of statements 175-182, wherein the agent capable of inhibiting or reversing EMT is administered in combination with a further cancer treatment.
185. A method according to statement 184, wherein the further cancer treatment is selected from:
   (i) alkylating agents, including alkyl sulfonates such as busulfan;
   (ii) nitrogen mustards such as chlorambucil, cyclophosphamide, estramustine, ifosfamide, mechlorethamine, melphalan, and uramustine;
   (iii) ethyleneimine derivatives such as thiotepa;
   (iv) nitrosoureas such as carmustine, lomustine, and streptozocin;
   (v) triazenes such as dacarbazine, procarbazine, and temozolamide;
   (vi) platinum compounds such as cisplatin, carboplatin, oxaliplatin, satraplatin, and picoplatin onnaplatin, tetraplatin, sprioplatin, iproplatin, chloro(diethylenediamino)-platinum (II) chloride, dichloro(ethylenediamino)-platinum (II), diamino(2-ethylmalonato)platinum (II), (1,2-diaminocyclohexane)malonatoplatinum (II), (4-carboxyphthalo)-(1,2-diaminocyclohexane)platinum (II), (1,2-diaminocyclohexane)-(isocitrato)platinum (II), and (1,2-diaminocyclohexane)-cis-(pyruvato)platinum (II);
   (vii) antimetabolites, including antifolates such as methotrexate, permetrexed, raltitrexed, and trimetrexate;
   (viii) pyrimidine analogues such as azacitidine, capecitabine, cytarabine, edatrexate, floxuridine, fluorouracil, gemcitabine, and troxacitabine;
   (ix) purine analogues such as cladribine, chlorodeoxyadenosine, clofarabine, fludarabine, mercaptopurine, pentostatin, and thioguanine;
   (x) natural products, including antitumor antibiotics such as bleomycin, dactinomycin, mithramycin, mitomycin, mitoxantrone, porfiromycin;
   (xi) anthracyclines such as daunorubicin, doxorubicin, epirubicin, idarubicin, and valrubicin;
   (xii) mitotic inhibitors such as the vinca alkaloids vinblastine, vinvesir, vincristine, vindesine, and vinorelbine;
   (xiii) enzymes such as L-asparaginase and PEG-L-asparaginase;
   (xiv) microtubule polymer stabilizers such as the taxanes paclitaxel and docetaxel;
   (xv) topoisomerase I inhibitors such as the camptothecins irinotecan and topotecan;
   (xvi) topoisomerase II inhibitors such as podophyllotoxin, amsacrine, etoposide, teniposide, losoxantrone and actinomycin;
   (xvii) hormones and hormone antagonists, including
      androgens such as fluoxymesterone and testolactone;
      antiandrogens such as bicalutamide, cyproterone, flutamide, and nilutamide;
      corticosteroids such as dexamethasone and prednisone;
      aromatase inhibitors such as aminoglutethimide, anastrozole, exemestane, formestane, and letrozole;
      estrogens such as diethylstilbestrol;
      antiestrogens such as fulvestrant, raloxifene, tamoxifen, and toremifine;
      luteinising hormone-releasing hormone (LHRH) agonists and antagonists such as abarelix, buserelin, goserelin, leuprolide, histrelin, desorelin, nafarelin acetate and triptorelin;
      progestins such as medroxyprogesterone acetate and megestrol acetate; and
      thyroid hormones such as levothyroxine and liothyronine;
   (xviii) PKB pathway inhibitors, including perifosine, enzastaurin hydrochloride, and triciribine;
   (xix) P13K inhibitors such as semaphore and SF1126, and MTOR inhibitors such as rapamycin and analogues;
   (xx) CDK inhibitors, including seliciclib, alvocidib, and 7-hydroxystaurosporine;
   (xxi) COX-2 inhibitors, including celecoxib;
   (xxii) HDAC inhibitiors, including trichostatin A, suberoylanilide hydroxamic acid, and chlamydocin;
   (xxiii) DNA methylase inhibitors, including temozolomide, and miscellaneous agents, including altretamine, arsenic trioxide, thalidomide, lenalidomide, gallium nitrate, levamisole, mitotane, hydroxyurea, octreotide, procarbazine, suramin, photodynamic compounds such as methoxsalen and sodium porfimer;
   (xxiv) proteasome inhibitors such as bortezomib;
   (xxv) molecular targeted therapy agents including: functional therapeutic agents, including gene therapy agents and antisense therapy agents;
   (xxvi) tyrosine kinase inhibitors such as erlotinib hydrochloride, gefitinib, imatinib mesylate, and semaxanib;
   (xxvii) Raf inhibitors such as sorafenib, and gene expression modulators such as the retinoids and rexinoids, for example adapalene, bexarotene, trans-retinoic acid, 9-cis-retinoic acid, and N-(4-hydroxyphenyl)retinamide;
   (xxviii) phenotype-directed therapy agents, including monoclonal antibodies such as pembrolizumab, alemtuzumab, bevacizumab, cetuximab, ibritumomab tiuxetan, rituximab, and trastuzumab, immunotoxins such as gemtuzumab ozogamicin, radioimmunoconjugates such as I-tositumobab;
   (xxix) cancer vaccines;
   (xxx) biologic therapy agents including: interferons such as interferon-[alpha]2a and interferon-[alpha]2b, and interleukins such as aldesleukin, denileukin diftitox, and oprelvekin;
   (xxxi) anticancer therapies involving the use of protective or adjunctive agents, including: cytoprotective agents such as amifostine, and dexrazoxane, phosphonates such as pamidronate and zoledronic acid, and stimulating factors such as epoetin, darbeopetin, filgrastim, PEG-filgrastim, and sargramostim;
   (xxxii) Axl inhibitors such as 1-(6,7-dihydro-5H-benzo[6,7]cyclohepta[1,2-c]pyridazin-3-yl)-N3-((7-(S)-pyrrolidin-1-yl)-6,7,8,9-tetrahydro-5H-benzo[7]annulene-2-yl)-1H-1,2,4-triazole-3,5-diamine; and/or
   (xxxiii) further combination chemotherapeutic regimens, such as combinations of carboplatin/paclitaxel, capecitabine/docetaxel, fluorauracil/levamisole, fluorauracil/leucovorin, methotrexate/leucovorin, and trastuzumab/paclitaxel, alone or in further combination with carboplatin.
186. A method according to statement 184, wherein the further cancer treatment is a monoclonal antibody such as pembrolizumab, alemtuzumab, bevacizumab, cetuximab, ibritumomab tiuxetan, or rituximab, or is a B-Raf inhibitor such as debrafenib, a MEK inhibitor such as trametinib or a combination therapy of these agents.
187. A method according to statement 186, wherein the further cancer treatment is pembrolizumab.
188. A method according to statement 186, wherein the further cancer treatment is a combination therapy of debrafenib and trametinib.
189. A method according to any one of statements 101-186, wherein the subject is mammalian.
190. A method according to statement 189, wherein the subject is human.
191. A method according to any one of statements 101-190, wherein the predictive method is performed before the subject receives a treatment or course of treatment for melanoma.
192. A method according to any one of statement 101-191, wherein the predictive method is performed during or after a treatment or course of treatment for melanoma.
193. A method according to any one of statements 101-190 or 192, wherein the subject has previously been treated with an agent capable of inhibiting or reversing EMT or a chemotherapeutic agent.
194. A method according to statement 193, wherein the subject has previously been treated with an agent selected from: a PHGDH inhibitor, a Slfn11 inhibitor, an Axl inhibitor, or an Akt3 inhibitor.
195. A method according to any one of statements 193-194 wherein the subject has previously been treated with an agent that is different to the agent capable of inhibiting or reversing EMT or chemotherapeutic agent for which the subject's susceptibility is being predicted.
196. A method according to any one of statements 101-191, wherein the subject has not previously been treated with an agent capable of inhibiting or reversing EMT or a chemotherapeutic agent.
197. A method according to statement 196, wherein the subject has not previously been treated with an Axl inhibitor.
198. A method according to statement 196, wherein the subject has not previously been treated with an Akt3 inhibitor.
199. A method according to any one of statements 101-198, wherein the sample is selected from: blood, serum, plasma, tissue biopsy, culture supernatant, or urine.
200. A method according to statement 199, wherein the sample is a blood, serum, or plasma sample.
201. A method according to statement 200, wherein the sample is a serum sample.
202. A method according to any one of statements 104-201, wherein the method further comprises a subsequent step of assessing the activity, expression, or amount of one or more biomarker in the subject, in which a sample profile is obtained after contacting the subject with an agent capable of inhibiting or reversing EMT or a chemotherapeutic agent.
203. A method according to statement 103, wherein the sample profile is obtained after contacting the subject with an agent capable of inhibiting or reversing EMT or a chemotherapeutic agent.
204. A method according to statement 202 or 203, wherein the control profile is obtained from the same subject prior to contacting said subject with the agent capable of inhibiting or reversing EMT or a chemotherapeutic agent.
205. A method according to statement 204, wherein an increase in the activity, expression, or amount of one or more biomarker in the sample profile as compared with the control profile is indicative of higher susceptibility to treatment with an agent capable of inhibiting or reversing EMT or a chemotherapeutic agent.
206. A method according to statement 204, wherein a decrease in the activity, expression, or amount of one or more biomarker in the sample profile as compared with the control profile is indicative of higher susceptibility to treatment with an agent capable of inhibiting or reversing EMT or a chemotherapeutic agent.
207. A method according to statement 204, wherein an increase in the activity, expression, or amount of one or more biomarker in the sample profile as compared with the control profile is indicative of lower susceptibility to treatment with an agent capable of inhibiting or reversing EMT or a chemotherapeutic agent.
208. A method according to statement 204, wherein a decrease in the activity, expression, or amount of one or more biomarker in the sample profile as compared with the control profile is indicative of lower susceptibility to treatment with an agent capable of inhibiting or reversing EMT or a chemotherapeutic agent.
209. A method according to statement 205, wherein the one or more biomarker includes at least HB-EGF.
210. A method according to statement 205, wherein the one or more biomarker includes at least CgA.
211. A method according to statement 205, wherein the one or more biomarker includes at least Nr-CAM.
212. A method according to statement 205, wherein the one or more biomarker includes at least KIM-1.
213. A method according to statement 205, wherein the one or more biomarker includes at least CD40.
214. A method according to statement 205, wherein the one or more biomarker includes at least LAP TGF-b1.
215. A method according to statement 205, wherein the one or more biomarker includes at least EGF.
216. A method according to statement 205, wherein the one or more biomarker includes at least TATI.
217. A method according to statement 205, wherein the one or more biomarker includes at least Axl.
218. A method according to statement 205, wherein the one or more biomarker includes at least LDH.
219. A method according to any one of statements 202-218, wherein assessing the activity, expression, or amount of one or more biomarker in the subject, or in a sample derived from the subject, comprises:
   contacting a sample, or an extract from the sample, with at least 1, 2, 3, 4, 5, 6, 7, 8, 9, 10 or more reagents for detecting the activity, expression, or amount of one or more biomarkers, each of which is selective for a particular biomarker; and
   detecting and/or quantifying said reagents for detecting.
220. A method according to statement 219, wherein assessing the expression or amount of one or more biomarker in the subject, or in a sample derived from the subject, comprises:
   contacting a sample, or an extract from the sample, with at least 1, 2, 3, 4, 5, 6, 7, 8, 9, 10 or more specific binding members, each of which selectively binds to a respective biomarker protein or mRNA; and
   detecting and/or quantifying formation of a complex formed by said specific binding member and said biomarker protein or mRNA.
221. A method according to statement 220, wherein the specific binding member comprises an antibody molecule or binding fragment thereof.
222. A method according to statement 220 or 221, wherein detecting formation of a complex formed by the specific binding member and the biomarker protein or mRNA is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.
223. A method according to any one of statements 220-222 wherein the expression or amount of the one or more biomarker is determined as the absolute amount of the respective biomarker protein or mRNA in the sample.
224. A method according to any one of statements 220-222 wherein the expression or amount of the one or more biomarker is determined as a relative amount of the respective biomarker protein or mRNA in the sample,
   optionally wherein the relative amount is determined relative to a reference protein or mRNA in the sample, the expression of which is not altered by melanoma.
225. A method according to any one of statements 203-224, wherein the agent capable of inhibiting or reversing EMT or chemotherapeutic agent is an Axl inhibitor, Akt3 inhibitor, Slfn11 inhibitor, or PHGDH inhibitor.
226. A method according to statement 225, wherein the agent capable of inhibiting or reversing EMT or chemotherapeutic agent is an Axl inhibitor.
227. A method according to statement 226, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
228. A method according to statement 227, wherein the Axl inhibitor is BGB324/R428/bemcentinib.
229. A method according to statement 227, wherein the Axl inhibitor is an anti-Axl antibody described in WO2015/193428, WO2015/193430, WO2016/097370, or WO2016/166296.
230. A method according to statement 225, wherein the agent capable of inhibiting or reversing EMT or chemotherapeutic agent is an Akt3 inhibitor.
231. A method according to statement 230, wherein the Akt3 inhibitor is an Akt3 inhibitor disclosed in WO2016/102672.
232. A method according to any one of statements 225-231, wherein the agent capable of inhibiting or reversing EMT is administered as a single agent.
233. A method according to any one of statements 225-231, wherein the agent capable of inhibiting or reversing EMT is administered in combination with a further cancer treatment.
234. A method according to statement 233, wherein the further cancer treatment is selected from the further cancer treatments listed in statement 185.
235. A method according to statement 233, wherein the further cancer treatment is a monoclonal antibody such as pembrolizumab, alemtuzumab, bevacizumab, cetuximab, ibritumomab tiuxetan, or rituximab, or is a B-Raf inhibitor such as debrafenib, a MEK inhibitor such as trametinib or a combination therapy of these agents.
236. A method according to statement 235, wherein the further cancer treatment is pembrolizumab.
237. A method according to statement 235, wherein the further cancer treatment is a combination therapy of debrafenib and trametinib.
238. A method according to any one of statements 203-237, wherein the subject is mammalian.
239. A method according to statement 238, wherein the subject is human.
240. A method according to any one of statements 203-239, wherein the subject was selected for treatment with the agent capable of inhibiting or reversing EMT or a chemotherapeutic agent, using a predictive method according to any one of statements 101-202.
241. A method according to any one of statements 203-240, wherein the sample is selected from: blood, serum, plasma, tissue biopsy, culture supernatant, or urine.
242. A method according to statement 241, wherein the sample is a blood, serum, or plasma sample.
243. A method according to statement 242, wherein the sample is a serum sample.
244. A method according to any one of statements 1-243, wherein the method is performed in vitro or ex vivo.
201a. A method of selecting subjects having, suspected of having, or diagnosed with melanoma for treatment with an agent capable of inhibiting or reversing EMT or a chemotherapeutic agent, the method comprising:
   identifying subjects susceptible to treatment with an agent capable of inhibiting or reversing EMT or a chemotherapeutic agent using a method according to any one of statements 101-244; and
   selecting thus identified subjects for treatment.
202a. A method of selecting subjects having, suspected of having, or diagnosed with melanoma for continued treatment with an agent capable of inhibiting or reversing EMT or a chemotherapeutic agent, the method comprising:
   identifying subjects susceptible to treatment with an agent capable of inhibiting or reversing EMT or a chemotherapeutic agent using a method according to any one of statements 203-244; and
   selecting thus identified subjects for treatment.
203a. A method according to any one of statements 201a-202a, wherein treatment comprises administering to the subject a therapeutically effective amount of an agent capable of inhibiting or reversing EMT or a chemotherapeutic agent.
204a. A method according to statement 203a, wherein the treatment comprises an Axl inhibitor, Akt3 inhibitor, Slfn11 inhibitor, or PHGDH inhibitor.
205a. A method according to statement 204a, wherein the treatment comprises an Axl inhibitor.
206a. A method according to statement 205a, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
207a. A method according to statement 206a, wherein the Axl inhibitor is BGB324/R428/bemcentinib.
208a. A method according to statement 206a, wherein the Axl inhibitor is an anti-Axl antibody described in WO2015/193428, WO2015/193430, WO2016/097370, or WO2016/166296.
209a. A method according to statement 204a, wherein the treatment comprises an Akt3 inhibitor.
210a. A method according to statement 209a, wherein the Akt3 inhibitor is an Akt3 inhibitor disclosed in WO2016/102672.
211a. A method according to any one of statements 203a-210a, wherein the treatment is administered as a single agent.
212a. A method according to any one of statements 203a-211a, wherein the treatment is administered in combination with a further cancer treatment.
213a. A method according to statement 212a, wherein the further cancer treatment is selected from the further cancer treatments listed in statement 185.
214a. A method according to statement 212a, wherein the further cancer treatment is a monoclonal antibody such as pembrolizumab, alemtuzumab, bevacizumab, cetuximab, ibritumomab tiuxetan, or rituximab, or is a B-Raf inhibitor such as debrafenib, a MEK inhibitor such as trametinib or a combination therapy of these agents.
215a. A method according to statement 214a, wherein the further cancer treatment is pembrolizumab.
216a. A method according to statement 214a, wherein the further cancer treatment is a combination therapy of debrafenib and trametinib.
301. A diagnostic kit comprising 1, 2, 3, 4, 5, 6, 7, 8, 9, 10 or more reagents for detecting the activity, expression, or amount of one or more biomarkers, each of which is selective for a particular biomarker selected from the group consisting of: HB-EGF, AFP, CgA, Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH.
302. A test device comprising 1, 2, 3, 4, 5, 6, 7, 8, 9, 10 or more reagents for detecting the activity, expression, or amount of one or more biomarkers, each of which is selective for a particular biomarker selected from the group consisting of: HB-EGF, AFP, CgA, Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH.
303. A diagnostic kit or test device according to any one of statements 301 or 302, comprising:
   1, 2, 3, 4, 5, 6, 7, 8, 9, 10 or more specific binding members, each of which selectively binds to a biomarker selected from the group consisting of: HB-EGF, AFP, CgA, Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH; and
   one or more reagents for detecting said one or more specific binding members or one or more reagents for detecting and/or quantifying formation of a complex formed by said specific binding member and said biomarker.
304. A diagnostic kit or test device according to statement 303, wherein the specific binding member comprises an antibody molecule or binding fragment thereof.
305. A diagnostic kit or test device according to statement 303 or 304, wherein detecting formation of a complex formed by the specific binding member and the biomarker is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.
306. A diagnostic kit or test device according to any one of statements 301-305, wherein the kit or test device comprises no more than 15, 20, 25, 30, 40, or 50 reagents for detecting.
307. A diagnostic kit or test device according to any one of statements 301-306 for use in a method of predicting a cancer-related outcome in a subject.
308. A diagnostic kit for use, or test device for use, according to statement 307, wherein the cancer-related outcome is susceptibility to treatment with an agent capable of inhibiting or reversing EMT or a chemotherapeutic agent.
309. A diagnostic kit for use, or test device for use, according to statement 308, wherein the agent capable of inhibiting or reversing EMT or chemotherapeutic agent is an Axl inhibitor, Akt3 inhibitor, Slfn11 inhibitor, or PHGDH inhibitor.
310. A diagnostic kit for use, or test device for use, according to statement 308, wherein the agent capable of inhibiting or reversing EMT or chemotherapeutic agent is an Axl inhibitor.
311. A diagnostic kit for use, or test device for use, according to statement 310, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
312. A diagnostic kit for use, or test device for use, according to statement 311, wherein the Axl inhibitor is BGB324/R428/bemcentinib.
313. A diagnostic kit for use, or test device for use, according to statement 311, wherein the Axl inhibitor is an anti-Axl antibody described in WO2015/193428, WO2015/193430, WO2016/097370, or WO2016/166296.
314. A diagnostic kit for use, or test device for use, according to statement 308, wherein the agent capable of inhibiting or reversing EMT or chemotherapeutic agent is an Akt3 inhibitor.
315. A diagnostic kit for use, or test device for use, according to any one of statements 307-310, wherein the subject has, is suspected of having, or has been diagnosed with melanoma.
316. A diagnostic kit for use, or test device for use, according to any one of statements 307-315, wherein the method is a method according to any one of statements 101-244.
317. Use of a reagent for detecting the activity, expression, or amount of a biomarker selected from the group consisting of: HB-EGF, AFP, CgA, Nr-CAM, KIM-1, CD40, LAP TGF-b1, EGF, IL-2 receptor alpha, hCG, TARO, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH in a method of predicting a cancer-related outcome in a subject.
318. Use of 1, 2, 3, 4, 5, 6, 7, 8, 9, 10 or more reagents for detecting the activity, expression, or amount a biomarker, each of which is selective for a particular biomarker selected from the group consisting of: HB-EGF, AFP, CgA, Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH, in a method of manufacture of a diagnostic kit or test device for use in a method of predicting a cancer-related outcome in a subject.
319. A use according to statement 317 or 318, wherein the reagent for detecting comprises a specific binding member selective for a particular biomarker.
320. A use according to statement 317 or 318, wherein the method of predicting a cancer-related outcome in a subject is a method according to any one of statements 101-244.
401. Use of one or more of: HB-EGF, AFP, CgA, Nr-CAM, KIM-1, CD40, LAP TGF-b1, EGF, IL-2 receptor alpha, hCG, TARC, CD40-L, VEGFR-3, TATI, MMP-7, Leptin, Apo A-I, Periostin, ANG-2, Cystatin-C, Hepsin Apo C-I, IL-1RI, Eotaxin-2, MPO, FRTN, Axl, and/or LDH as a biomarker for determining if a subject is susceptible to treatment with an agent capable of inhibiting or reversing EMT or a chemotherapeutic agent.
402. A use according to statement 401, wherein:
   (i) a higher activity, expression, or amount of one or more of said biomarkers in the subject, or sample derived from the subject, compared with a control profile for the respective biomarker is indicative of susceptibility to treatment with an agent capable of inhibiting or reversing EMT or a chemotherapeutic agent; or
   (ii) a lower activity, expression, or amount of one or more of said biomarkers in the subject, or sample derived from the subject, compared with a control profile for the respective biomarker is indicative of susceptibility to treatment with an agent capable of inhibiting or reversing EMT or a chemotherapeutic agent.
403. A use according to statement 401, wherein:
   (i) an increase in the activity, expression, or amount of one or more biomarker relative to a control profile for the respective biomarker after contacting the subject with a cancer therapeutic agent is indicative of increased susceptibility to treatment with an agent capable of inhibiting or reversing EMT or a chemotherapeutic agent; or
   (ii) a decrease in the activity, expression, or amount of one or more biomarker relative to a control profile for the respective biomarker after contacting the subject with a cancer therapeutic agent is indicative of increased susceptibility to treatment with an agent capable of inhibiting or reversing EMT or a chemotherapeutic agent.
404. A use according to any one of statements 401-403, wherein the use is as a biomarker in a method according to any one of statements 101-244
501. A method of treating a subject having, suspected of having, or diagnosed with melanoma, the method comprising:
   identifying a subject susceptible to treatment using a method according to any one of statements 101-244; and
   administering to the subject a therapeutically effective amount of an agent capable of inhibiting or reversing EMT or a chemotherapeutic agent.
502. A method of treating a subject having, suspected of having, or diagnosed with melanoma, the method comprising:
   (a) obtaining a sample from the subject;
   (b) determining if the subject is susceptible to treatment with an agent capable of inhibiting or reversing EMT or a chemotherapeutic agent using a method according to any one of statements 101-244;
   (c) administering to the subject a therapeutically effective amount of an agent capable of inhibiting or reversing EMT or a chemotherapeutic agent.
503. A method of treating a subject having, suspected of having, or diagnosed with melanoma, the method comprising administering a therapeutically effective amount of an agent capable of inhibiting or reversing EMT or chemotherapeutic agent to a subject identified as susceptible to said treatment using a method according to any one of statements 101-244.
504. A method of treating a subject having, suspected of having, or diagnosed with melanoma, the method comprising:
   administering to the subject a therapeutically effective amount of an agent capable of inhibiting or reversing EMT or a chemotherapeutic agent; and
   determining if the subject is susceptible to said treatment using a method according to any one of statements 203-244.
505. A method according to statement 504, wherein the method comprises administering to the subject one or more further therapeutically effective amounts of an agent capable of inhibiting or reversing EMT or a chemotherapeutic agent provided that the subject is identified as susceptible to said treatment.
506. An agent capable of inhibiting or reversing EMT or chemotherapeutic agent for use in a method of treating a subject having, suspected of having, or diagnosed with melanoma, wherein the method is a method according to any one of statements 501-505.
507. Use of an agent capable of inhibiting or reversing EMT or chemotherapeutic agent for use in a method of manufacture of a medicament for use in a method of treating a subject having, suspected of having, or diagnosed with melanoma,
   wherein the method of treatment is a method according to any one of statements 501-505.
508. A method, agent for use, or use according to any one of statements 501-507, wherein the agent capable of inhibiting or reversing EMT or chemotherapeutic agent comprises an Axl inhibitor, Akt3 inhibitor, Slfn11 inhibitor, or PHGDH inhibitor.
509. A method, agent for use, or use according to statement 508, wherein the agent capable of inhibiting or reversing EMT or chemotherapeutic agent comprises an Axl inhibitor.
510. A method, agent for use, or use according to statement 509, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
511. A method, agent for use, or use according to statement 510, wherein the Axl inhibitor is BGB324/R428/bemcentinib.
512. A method, agent for use, or use according to statement 510, wherein the Axl inhibitor is an anti-Axl antibody described in WO2015/193428, WO2015/193430, WO2016/097370, or WO2016/166296.
512. A method, agent for use, or use according to statement 508, wherein the agent capable of inhibiting or reversing EMT or chemotherapeutic agent comprises an Akt3 inhibitor.
513. A method, agent for use, or use according to statement 512, wherein the Akt3 inhibitor is an Akt3 inhibitor disclosed in WO2016/102672.
514. A method, agent for use, or use according to any one of statements 501-513, wherein the agent capable of inhibiting or reversing EMT or chemotherapeutic agent is administered as a single agent.
515. A method, agent for use, or use according to any one of statements 501-513, wherein the agent capable of inhibiting or reversing EMT or chemotherapeutic agent is administered in combination with a further cancer treatment.
516. A method, agent for use, or use according to statement 515, wherein the further cancer treatment is selected from the further cancer treatments listed in statement 185.
517. A method, agent for use, or use according to statement 515, wherein the further cancer treatment is a monoclonal antibody such as pembrolizumab, alemtuzumab, bevacizumab, cetuximab, ibritumomab tiuxetan, or rituximab, or is a B-Raf inhibitor such as debrafenib, a MEK inhibitor such as trametinib or a combination therapy of these agents.
518. A method, agent for use, or use according to statement 517, wherein the further cancer treatment is pembrolizumab.
519. A method, agent for use, or use according to statement 517, wherein the further cancer treatment is a combination therapy of debrafenib and trametinib.

Facet 5: Triple Negative Breast Cancer (TNBC)

Triple-negative breast cancers (TNBC) are a heterogeneous group of breast cancers that do not express the genes for estrogen receptor (ER), progesterone receptor (PR) and Her2/neu. Because most hormone therapies target one of these receptors, triple-negative breast cancers are among the most difficult to treat, typically requiring combination therapy. In view of the significant degree of heterogeneity in triple-negative breast cancers and patient response to drug therapies, there remains a need for targeted therapies for triple-negative breast cancers (Jhan et al, 2017).

Accordingly, robust biomarkers for predicting cancer-related outcomes in a subject having triple-negative breast cancer, for example susceptibility to treatment with an agent capable of inhibiting or reversing EMT or a chemotherapeutic agent, would be useful in identifying subjects who are most likely to respond to or benefit from treatment with a particular agent/regimen.

A “responder” subject as described herein is a subject who is likely to, or who does, respond to treatment (or benefit from treatment) with an agent capable of inhibiting or reversing EMT or a chemotherapeutic agent, for example the small molecule Axl kinase inhibitor BGB324/R428/bemcentinib when administered either as a single agent or in combination with another cancer treatment.

Correspondingly, a “non-responder” subject as described herein is a subject who is not likely to, or who does not, respond to treatment (or benefit from treatment) with an agent capable of inhibiting or reversing EMT or a chemotherapeutic agent, for example the small molecule Axl kinase inhibitor BGB324/R428/bemcentinib when administered either as a single agent or in combination with another cancer treatment.

To “respond to treatment” or “benefit from treatment” as used herein means to experience an overall clinical benefit from the treatment. This overall clinical benefit can be any of: prolonged survival, partial or complete disease remission, slowing or absence of disease progression, tumour shrinkage (for example, a reduction in tumour volume of 5, 10, 20, 30, 40% or more), reduction in tumour burden (for example, a reduction in tumour burden of 5, 10, 20, 30, 40% or more), slowing or absence of tumour enlargement, slowing or absence of increase in tumour burden, improved quality of life, progression-free survival, overall survival, or any other positive patient outcome. Suitable methods for determining tumour volume are well known to the skilled person, for example, using: computed tomography (CT), or magnetic resonance imaging (MRI) imaging technologies; X-ray imaging, for example, mammography; ultrasound imaging; nuclear imaging, for example positron emission tomography (PET), PET/CT scans, bone scans, gallium scans, or metaiodobenzylguanidine (MIBG) scans; bioluminescence imaging (BLI); fluorescence imaging (FLI); BD ToF (infrared-based 3D Time-of-Flight camera) imaging.

Biomarkers

The term “marker” or “biomarker” is used herein to refer to a gene or protein whose expression in a subject or sample derived from a subject is altered or modulated, for example, up or down regulated, in cancer. Where the biomarker is a protein, modulation or alteration of expression encompasses modulation through different post-translational modifications.

In accordance with any aspect of this facet of the present disclosure the biomarkers described herein may be defined as follows, wherein:
an Axl biomarker comprises an amino acid sequence having at least 70%, 80%, 90%, 95%, 99% or 100% sequence identity with the full-length of the amino acid sequence disclosed at UniProt accession no: P30530-1 or P30530-2 (entry version 196) or a fragment thereof;
an Interleukin-6 receptor subunit beta (IL-6R beta) biomarker comprises an amino acid sequence having at least 70%, 80%, 90%, 95%, 99% or 100% sequence identity with the full-length of the amino acid sequence disclosed at UniProt accession no: P40189-1 or P40189-2 (entry version 209) or a fragment thereof;
an Intercellular Adhesion Molecule 1 (ICAM-1) biomarker comprises an amino acid sequence having at least 70%, 80%, 90%, 95%, 99% or 100% sequence identity with the full-length of the amino acid sequence disclosed at UniProt accession no: P05362 (entry version 221) or a fragment thereof;
a Macrophage Inflammatory Protein-1 beta (MIP-1 beta) biomarker comprises an amino acid sequence having at least 70%, 80%, 90%, 95%, 99% or 100% sequence identity with the full-length of the amino acid sequence disclosed at UniProt accession no: P13236 (entry version 187) or a fragment thereof;
a Periostin biomarker comprises an amino acid sequence having at least 70%, 80%, 90%, 95%, 99% or 100% sequence identity with the full-length of the amino acid sequence disclosed at UniProt accession no: Q15063-1, Q15063-2, Q15063-3, Q15063-4, Q15063-5, Q15063-6, Q15063-7 (entry version 160) or a fragment thereof;
an Interleukin-23 (IL-23) biomarker comprises an amino acid sequence having at least 70%, 80%, 90%, 95%, 99% or 100% sequence identity with the full-length of the amino acid sequence disclosed at UniProt accession no: Q9NPF7 (entry version 133) or a fragment thereof;
a Folate receptor gamma (FOLR3) biomarker comprises an amino acid sequence having at least 70%, 80%, 90%, 95%, 99% or 100% sequence identity with the full-length of the amino acid sequence disclosed at UniProt accession no: P41439-1, P41439-2, or P41439-3 (entry version 127) or a fragment thereof;
a Clusterin (CLU) biomarker comprises an amino acid sequence having at least 70%, 80%, 90%, 95%, 99% or 100% sequence identity with the full-length of the amino acid sequence disclosed at UniProt accession no: P10909-1, P10909-2, P10909-3, P10909-4, or P10909-5 (entry version 209) or a fragment thereof; and/or
a Fas Ligand (FasL) biomarker comprises an amino acid sequence having at least 70%, 80%, 90%, 95%, 99% or 100% sequence identity with the full-length of the amino acid sequence disclosed at UniProt accession no: P48023-1 or P48023-2 (entry version 197) or a fragment thereof.

The biomarkers described herein may also be defined as a nucleic acid (either DNA or RNA) having at least 70%, 80%, 90%, 95%, 99% or 100% sequence identity with the full-length nucleic acid sequence encoding any of the above amino acid sequences.

Identity may be as defined using sequence comparisons made using FASTA and FASTP (see Pearson & Lipman, 1988. Methods in Enzymology 183: 63-98). Parameters are preferably set, using the default matrix, as follows: Gapopen (penalty for the first residue in a gap): −12 for proteins/-16 for DNA; Gapext (penalty for additional residues in a gap): −2 for proteins/-4 for DNA; KTUP word length: 2 for proteins/6 for DNA.

Pre-Treatment Predictive Biomarkers and Methods

By analysing patient data obtained from clinical trial BGBC007 (NCT03184558), the authors have discovered that, prior to treatment of subjects having TNBC, certain of the biomarkers described herein are present in the serum of “responder” subjects at higher levels than in the serum of “non-responder” subjects—see Example 3. Similarly, prior to treatment of the subject certain of the biomarkers described herein are present in the serum of “responder” subjects at lower levels than in the serum of “non-responder” subjects. Thus, by assessing the activity, expression, or amount of one or more of the biomarkers described herein prior to treatment with an agent capable of inhibiting or reversing EMT or a chemotherapeutic agent it is possible to determine the likelihood of that subject being susceptible or responsive to treatment with said agent.

Accordingly, in a first aspect of this facet, the disclosure provides a method of predicting a cancer-related outcome in a subject having, suspected of having, or diagnosed with triple-negative breast cancer (TNBC), the method comprising assessing the activity, expression, or amount of one or more of the biomarkers described herein in the subject, or in a sample derived from the subject.

In some embodiments the one or more biomarker is selected from the group consisting of: Axl, Interleukin-6 receptor subunit beta (IL-6R beta), Intercellular Adhesion Molecule 1 (ICAM-1), Macrophage Inflammatory Protein-1 beta (MIP-1 beta), Periostin, Interleukin-23 (IL-23), Folate receptor gamma (FOLR3), Clusterin (CLU), and/or Fas Ligand (FasL).

A “cancer-related outcome” as described herein is a clinical prediction or prognosis associated with cancer. In some embodiments the cancer-related outcome is susceptibility to treatment with an agent capable of inhibiting or reversing EMT or a chemotherapeutic agent.

In some embodiments the method comprises assessing the activity, expression, or amount of one or more of the biomarkers described herein in the subject, or in a sample derived from the subject, to obtain a sample profile of the one or more biomarker; and, making a prediction based on the sample profile of the one or more biomarker.

A “sample profile” as used herein is a profile of numerical values or numerical ranges corresponding to the activity, expression, or amount of respective biomarkers determined in the subject or sample derived from the subject.

In some embodiments of the method the prediction is made by comparing the sample profile to a control profile. A “control profile” as used herein is a profile of numerical values or numerical ranges corresponding to the activity, expression, or amount of the same biomarkers as for the sample profile, determined in a control subject or population of control subjects. Suitable control profiles are described in more detail below.

Prior to treatment of a subject, certain of the biomarkers described herein have been found to exhibit up-regulated expression in “responder” subjects—that is, certain of the biomarkers described herein have been found to be present in the serum of “responder” subjects at higher levels than in the serum of “non-responder” subjects. Similarly, prior to treatment of a subject, certain of the biomarkers described herein have been found to exhibit down-regulated expression in “responder” subjects—that is, certain of the biomarkers described herein have been found to be present in the serum of “responder” subjects at lower levels than in the serum of “non-responder” subjects.

This difference in expression level between “responder” and “non-responder” subjects can also be expressed in the opposite way. That is to say, prior to treatment of a subject, certain of the biomarkers described herein have been found to exhibit down-regulated expression in “non-responder” subjects—that is, certain of the biomarkers described herein have been found to be present in the serum of “non-responder” subjects at lower levels than in the serum of “responder” subjects. Similarly, prior to treatment of a subject, certain of the biomarkers described herein have been found to exhibit up-regulated expression in “non-responder” subjects—that is, certain of the biomarkers described herein have been found to be present in the serum of “non-responder” subjects at higher levels than in the serum of “responder” subjects.

The discovery of these differential patterns of expression mean that, by assessing the activity, expression, or amount of one or more of the biomarkers described herein prior to treatment of a subject with an agent, it is possible to determine the likelihood of that subject being susceptible or responsive to treatment with said agent. Accordingly, in particularly preferred embodiments the sample profile is obtained before the subject is contacted with or administered the agent capable of inhibiting or reversing EMT or a chemotherapeutic agent.

A subject who is “susceptible to treatment” with an agent capable of inhibiting or reversing EMT or a chemotherapeutic agent is one who is likely to, or who does, respond to treatment (or benefit from treatment) with an agent capable of inhibiting or reversing EMT or a chemotherapeutic agent. That is, a subject who is “susceptible to treatment” with an agent is a “responder” subject for that agent, as defined above. A prediction that is “indicative of susceptibility” is one which suggests or points towards the likelihood that a subject will respond to or benefit from treatment.

In some embodiments the predictive methods of the disclosure comprise assessing the activity, expression, or amount of one or more of the biomarkers described herein in the subject, or in a sample derived from the subject, to obtain a sample profile of the one or more biomarker; and, making a prediction based on the sample profile of the one or more biomarker. In some embodiments of the method the prediction is made by comparing the sample profile to a control profile, wherein the control profile is a profile of numerical values or numerical ranges corresponding to the activity, expression, or amount of the same biomarkers as for the sample profile, determined in a control subject or population of control subjects.

In some embodiments the control profile may be obtained from a population of control subjects having TNBC. In some embodiments the control profile may be obtained from a population of control subjects not having TNBC. In other embodiments the control profile may be obtained from a control subject or population of control subjects having TNBC and previously found to lack susceptibility to treatment with an agent capable of inhibiting or reversing EMT or a chemotherapeutic agent (that is, non-responder subjects).

In some embodiments the control profile may a predetermined profile of biomarker expression, activity, or amount values, for example a profile of “mean” values, “threshold” values, or “standard ranges” of values. This predetermined profile of biomarker expression, activity, or amount values can be obtained, for example, from a control subject or population of control subjects having TNBC, a control subject or population of control subjects not having TNBC, or a control subject or population of control subjects having TNBC and previously found to lack susceptibility to treatment with an agent capable of inhibiting or reversing EMT or a chemotherapeutic agent (that is, non-responder subjects).

In some other embodiments the control profile may be obtained from a control subject or population of control subjects having TNBC and previously found to be susceptible to treatment with an agent capable of inhibiting or reversing EMT or a chemotherapeutic agent (that is, responder subjects). In some embodiments the control profile may a predetermined profile of biomarker expression, activity, or amount values, for example a profile of “threshold”, values or “standard ranges” of values. This predetermined profile of biomarker expression, activity, or amount values can be obtained, for example, from a control subject or population of control subjects having TNBC and previously found to be susceptible to treatment with an agent capable of inhibiting or reversing EMT or a chemotherapeutic agent (that is, responder subjects).

In some other embodiments the control profile may be obtained from a control sample having a known amount (for example a “threshold” amount) of the same biomarkers as will be determined in the sample profile. This threshold amount can be obtained, for example, from non-responder or responder subjects as outlined above.

The skilled person is readily able to determine suitable “mean”, “threshold”, or “standard range” values for a particular biomarker.

For example, a “mean” value may be determined by: assessing the activity, expression, or amount of a particular biomarker in a population of subjects having (or not having) TNBC; and, averaging the biomarker activity, expression, or amount determined to determine a “mean” value.

For example, a “mean” value may be determined by: assessing the activity, expression, or amount of a particular biomarker in a population of subjects; determining which subjects in the population are susceptible to treatment with an agent capable of inhibiting or reversing EMT or a chemotherapeutic agent; and, averaging the biomarker activity, expression, or amount determined in subjects found to be susceptible to treatment with an agent capable of inhibiting or reversing EMT or a chemotherapeutic agent (that is, responder subjects) to determine a “mean” value.

For example, a “threshold” value may be determined by: assessing the activity, expression, or amount of a particular biomarker in a population of subjects; determining which subjects in the population are susceptible to treatment with an agent capable of inhibiting or reversing EMT or a chemotherapeutic agent; and, selecting as appropriate the highest or lowest biomarker activity, expression, or amount determined in a subject found to be susceptible to treatment with an agent capable of inhibiting or reversing EMT or a chemotherapeutic agent (that is, a responder subject) as the “threshold value”.

For example, a “standard range” of values may be determined by: assessing the activity, expression, or amount of a particular biomarker in a population of subjects having (or not having) TNBC; and, determining a “standard range” of values based on this assessment.

For example, a “standard range” of values may be determined by: assessing the activity, expression, or amount of a particular biomarker in a population of subjects; determining which subjects in the population are susceptible to treatment with an agent capable of inhibiting or reversing EMT or a chemotherapeutic agent; and, determining a “standard range” of values for subjects found to be susceptible to treatment with an agent capable of inhibiting or reversing EMT or a chemotherapeutic agent (that is, a responder subject) based on this assessment.

Where the control profile is a predetermined profile of biomarker expression, activity, or amount values, this may be provided as a “look-up table” or data record. Predetermined control profiles of this type may be a profile of numerical values or numerical ranges corresponding to a measure of central tendency (such as an average, median or mean) amount of the respective biomarkers determined in a plurality of control subjects (for example non-responder or responder subjects as outlined above). The skilled person is readily able to determine both a measure of central tendency and a measure of variability within a population using standard protein quantification techniques and suitable statistical tools.

Accordingly, in embodiments in which the control profile is: obtained from a population of control subjects having TNBC; obtained from a population of control subjects not having TNBC; obtained from a control subject or population of control subjects having TNBC and previously found to lack susceptibility to treatment with an agent capable of inhibiting or reversing EMT or a chemotherapeutic agent (that is non-responder subjects); a predetermined profile of biomarker expression, activity, or amount values (for example a profile of “average, median, or mean” values or “standard ranges” of values) obtained from non-responder subjects; obtained from a control sample having a known “average, median, or mean” value of biomarkers indicative of non-responder subjects; a predetermined profile of biomarker expression, activity, or amount “threshold” values obtained from responder subjects; or, obtained from a control sample having a known “threshold” value of biomarkers indicative of responder subjects; then, a higher activity, expression, or amount of one or more of the biomarkers described herein in a sample profile as compared with the control profile may be indicative of susceptibility to treatment with an agent capable of inhibiting or reversing EMT or a chemotherapeutic agent. Similarly, a lower activity, expression, or amount of one or more of the biomarkers described herein in the sample profile as compared with a control profile may be indicative of susceptibility to treatment with an agent capable of inhibiting or reversing EMT or a chemotherapeutic agent.

In some such embodiments, the method may comprise assessing the activity, expression, or amount of one or more of biomarker selected from: IL-23, FOLR3, CLU, and/or FasL; wherein a higher activity, expression, or amount of one or more of said biomarkers in the sample profile as compared with the control profile is indicative of susceptibility to treatment with an agent capable of inhibiting or reversing EMT or a chemotherapeutic agent.

In some such embodiments, the method may comprise assessing the activity, expression, or amount of one or more of biomarker selected from: Axl, IL-6R beta, ICAM-1, MIP-1 beta, and/or Periostin; wherein a lower activity, expression, or amount of one or more of said biomarkers in the sample profile as compared with the control profile is indicative of susceptibility to treatment with an agent capable of inhibiting or reversing EMT or a chemotherapeutic agent.

In some such embodiments: a lower activity, expression, or amount of Axl; and/or a lower activity, expression, or amount of IL-6R beta; and/or a lower activity, expression, or amount of ICAM-1; and/or a lower activity, expression, or amount of MIP-1 beta; and/or a lower activity, expression, or amount of Periostin; and/or a higher activity, expression, or amount of IL-23; and/or a higher activity, expression, or amount of FOLR3; and/or a higher activity, expression, or amount of CLU; and/or a higher activity, expression, or amount of FasL; in the sample profile as compared with the control profile is indicative of susceptibility to treatment with an agent capable of inhibiting or reversing EMT or a chemotherapeutic agent.

In embodiments in which the control profile is: obtained from a control subject or population of control subjects having TNBC and previously found to be susceptible to treatment with an agent capable of inhibiting or reversing EMT or a chemotherapeutic agent (that is, responder subjects); then, if the expression, activity, or amount values in the sample profile are above a “threshold” value typical of a responder subject (that is, a subject having TNBC and previously found to be susceptible to treatment with an agent capable of inhibiting or reversing EMT or a chemotherapeutic agent) this is indicative of susceptibility to treatment with an agent capable of inhibiting or reversing EMT or a chemotherapeutic agent. Similarly, if the expression, activity, or amount values in the sample profile are below the “threshold” value typical of a responder subject (that is, a subject having TNBC and previously found to be susceptible to treatment with an agent capable of inhibiting or reversing EMT or a chemotherapeutic agent) this is indicative of a lack of susceptibility to treatment with an agent capable of inhibiting or reversing EMT or a chemotherapeutic agent.

In embodiments in which the control profile is: a predetermined profile of “standard ranges” of biomarker expression, activity, or amount values obtained from responder subjects; then, if the expression, activity, or amount values in the sample profile are within the “standard range” of values typical of a responder subject (that is, a subject having TNBC and previously found to be susceptible to treatment with an agent capable of inhibiting or reversing EMT or a chemotherapeutic agent) this is indicative of susceptibility to treatment with an agent capable of inhibiting or reversing EMT or a chemotherapeutic agent. Similarly, if the expression, activity, or amount values in the sample profile are outside the “standard range” of values typical of a responder subject (that is, a subject having TNBC and previously found to be susceptible to treatment with an agent capable of inhibiting or reversing EMT or a chemotherapeutic agent) this is indicative of a lack of susceptibility to treatment with an agent capable of inhibiting or reversing EMT or a chemotherapeutic agent.

In embodiments in which the control profile is: a predetermined profile of “standard ranges” of biomarker expression, activity, or amount values obtained from non-responder subjects; then, if the expression, activity, or amount values in the sample profile are within the “standard range” of values typical of a non-responder subject (that is, a subject having TNBC and previously found to lack susceptibility to treatment with an agent capable of inhibiting or reversing EMT or a chemotherapeutic agent) this is indicative of a lack of susceptibility to treatment with an agent capable of inhibiting or reversing EMT or a chemotherapeutic agent. Similarly, if the expression, activity, or amount values in the sample profile are outside the “standard range” of values typical of a non-responder subject (that is, a subject having TNBC and previously found to lack susceptibility to treatment with an agent capable of inhibiting or reversing EMT or a chemotherapeutic agent) this may be indicative of susceptibility to treatment with an agent capable of inhibiting or reversing EMT or a chemotherapeutic agent.

In embodiments in which the control profile is: obtained from a population of control subjects not having TNBC; then, if the expression, activity, or amount values in the sample profile are outside a “standard range” of values typical of subjects not having TNBC, this is indicative of susceptibility to treatment with an agent capable of inhibiting or reversing EMT or a chemotherapeutic agent. Similarly, if the expression, activity, or amount values in the sample profile are within a “standard range” of values typical of subjects not having TNBC, this is indicative of a lack of susceptibility to treatment with an agent capable of inhibiting or reversing EMT or a chemotherapeutic agent. In such cases, an expression, activity or amount value may be outside the “standard range” of values when it is either above the upper limit of normal or below the lower limit of normal for a given biomarker. The upper limit of normal and lower limit of normal are the limits of the normal distribution of a given biomarker in a population of control subjects not having TNBC.

The skilled person is readily able to determine whether the sample profile is indicative of susceptibility or lack of susceptibility to treatment with an agent capable of inhibiting or reversing EMT or a chemotherapeutic agent using suitable statistical tools and comparison with the appropriate control.

In some embodiments the sample profile may be compared with two or more control profiles as described above.

As used herein, the term “one or more” means 1, 2, 3, 4, 5, 6, 7, 8, 9, 10, 11, 12, 13, 14, 15, 16, 17, 18, 19, or 20 of the biomarkers described herein. In some embodiments the one or more biomarker is Axl. In some embodiments the one or more biomarker is IL-6R beta. In some embodiments the one or more biomarker is ICAM-1. In some embodiments the one or more biomarker is MIP-1 beta. In some embodiments the one or more biomarker is Periostin. In some embodiments the one or more biomarker is IL-23. In some embodiments the one or more biomarker is FOLR3. In some embodiments the one or more biomarker is CLU. In some embodiments the one or more biomarker is FasL.

In some embodiments the method may comprise assessing the activity, expression, or amount of at least 2, 3, 4, 5, 6, 7, 8, 9, 10, 11, 12, 13, 14, 15, 20 of said biomarkers in the subject, or in a sample derived from the subject. In some embodiments the method may comprise assessing the activity, expression, or amount of Axl and at least 1, 2, 3, 4, 5, 6, 7, 8, 9, 10, 11, 12, 13, 14, 15, 20 further biomarkers described herein in the subject, or in a sample derived from the subject.

Subject

A “subject” as referred to herein is any species of animal classified as a mammal, and includes but is not restricted to, domestic and farm animals, primates and humans. In some preferred embodiments the subject is a human of any sex or race. In some embodiments, the human is an adult human.

In some embodiments the predictive methods described herein are performed before the subject receives a treatment or course of treatment for TNBC. In other embodiments the predictive method is performed during or after a treatment or course of treatment for TNBC. In some embodiments the treatment or course of treatment for TNBC is an agent capable of inhibiting or reversing EMT. In other embodiments the treatment or course of treatment for TNBC is a chemotherapeutic treatment that is not an agent capable of inhibiting or reversing EMT.

In some embodiments of the predictive methods described herein the subject has previously been treated with an agent capable of inhibiting or reversing EMT or a chemotherapeutic agent. In some embodiments the subject has previously been treated with an agent selected from: a PHGDH inhibitor, a Slfn11 inhibitor, an Axl inhibitor, or an Akt3 inhibitor.

In some embodiments of the predictive methods described herein the subject has previously been treated with an agent that is different to the agent capable of inhibiting or reversing EMT or chemotherapeutic agent for which the subject's response is being predicted. That is, the subject has previously been treated with an agent that is not the agent which the predictive method is determining the subject's susceptibility to. Accordingly, in some embodiments, the subject has not previously been treated with an agent capable of inhibiting or reversing EMT or a chemotherapeutic agent. In some preferred embodiments, the subject has not previously been treated with an Axl inhibitor. In other preferred embodiments, the subject has not previously been treated with an Akt3 inhibitor.

Post-Treatment Changes in Biomarkers

In some embodiments of this aspect of the disclosure, the predictive method further comprises a subsequent step of assessing the activity, expression, or amount of one or more biomarker in the subject, or in a sample derived from the subject, to obtain a second sample profile of one or more of the biomarkers described herein after the subject has received a treatment or course of treatment an agent capable of inhibiting or reversing EMT or a chemotherapeutic agent.

That is, in some embodiments, the predictive method comprises:
(a) determining if a subject is susceptible to treatment with an agent capable of inhibiting or reversing EMT or a chemotherapeutic agent by assessing the activity, expression, or amount of one or more biomarker in the subject, as outlined above;
(b) administering to the subject a therapeutically effective amount of an agent capable of inhibiting or reversing EMT or a chemotherapeutic agent; and
(c) subsequently assessing the activity, expression, or amount of one or more biomarker in the subject, or in a sample derived from the subject.

In some embodiments, the predictive method comprises:
(a) determining if a subject is susceptible to treatment with an agent capable of inhibiting or reversing EMT or a chemotherapeutic agent by assessing the activity, expression, or amount of one or more biomarker in the subject, or in a sample derived from the subject, wherein the one or more biomarker is selected from the group consisting of: Axl, IL-6R beta, ICAM-1, MIP-1 beta, Periostin, IL-23, FOLR3, CLU, and/or FasL;
(b) administering to the subject a therapeutically effective amount of an agent capable of inhibiting or reversing EMT or a chemotherapeutic agent; and
(c) subsequently assessing the activity, expression, or amount of one or more biomarker in the subject, or in a sample derived from the subject.

In some preferred embodiments the methods comprise subsequently assessing the activity, expression, or amount of Axl in the subject, or in a sample derived from the subject. In some embodiments an increase in the activity, expression, or amount of Axl in the second sample profile as compared with a control profile after contacting the subject with a cancer therapeutic agent is indicative of higher susceptibility to treatment with an agent capable of inhibiting or reversing EMT or a chemotherapeutic agent. In some preferred embodiments the control profile is obtained from the same subject prior to treatment with an agent capable of inhibiting or reversing EMT or a chemotherapeutic agent.

Post-Treatment Predictive Biomarkers and Methods

By analysing patient data obtained from clinical trial BGBC007 (NCT03184558), the authors have discovered that, following treatment of subjects having TNBC with an agent capable of inhibiting or reversing EMT or a chemotherapeutic agent, the levels of certain of the biomarkers described herein increases in the serum of “responder” subjects but not in the serum of “non-responder” subjects. Similarly, following treatment with an agent capable of inhibiting or reversing EMT or a chemotherapeutic agent, the levels of certain of the biomarkers described herein decreases in the serum of “responder” subjects but not in the serum of “non-responder” subjects. Thus, by assessing the activity, expression, or amount of one or more of the biomarkers described herein prior to and following treatment with an agent capable of inhibiting or reversing EMT or a chemotherapeutic agent it is possible to determine or confirm that the subject is susceptible or responsive to treatment with said agent.

Accordingly, in an second aspect of this facet the disclosure provides a method of predicting a cancer-related outcome in a subject having, suspected of having, or diagnosed with triple-negative breast cancer (TNBC), the method comprising assessing the activity, expression, or amount of one or more of the biomarkers described herein in a subject, or in a sample derived from the subject after the subject has been contacted with an agent capable of inhibiting or reversing EMT or a chemotherapeutic agent. In some embodiments the cancer-related outcome is susceptibility to treatment with an agent capable of inhibiting or reversing EMT or a chemotherapeutic agent.

In some embodiments the method comprises assessing the activity, expression, or amount of one or more of the biomarkers described herein in the subject, or in a sample derived from the subject, to obtain a sample profile of the one or more biomarker; and, making a prediction based on the sample profile of the one or more biomarker. In some embodiments of the method the prediction is made by comparing the sample profile to a control profile. In preferred embodiments the sample profile is obtained after contacting the subject with an agent capable of inhibiting or reversing EMT or a chemotherapeutic agent.

A “control profile” as used herein is a profile of numerical values or numerical ranges corresponding to the activity, expression, or amount of the same biomarkers as for the sample profile, determined in a control subject or population of control subjects.

In preferred embodiments of this aspect of the disclosure the control profile is obtained from the same subject prior to treatment with an agent capable of inhibiting or reversing EMT or a chemotherapeutic agent. In some embodiments the control profile is obtained from a population of control subjects having TNBC. In other embodiments, the control profile is a predetermined profile of biomarker expression, activity, or amount values, for example a profile of “threshold” values or “standard ranges” of values.

Accordingly, in embodiments in which the control profile is obtained from the same subject prior to treatment with an agent capable of inhibiting or reversing EMT or a chemotherapeutic agent, an increase in the activity, expression, or amount of one or more biomarker in the sample profile as compared with a control profile after contacting the subject with an agent capable of inhibiting or reversing EMT or a cancer therapeutic agent may be indicative of susceptibility to treatment with an agent capable of inhibiting or reversing EMT or a chemotherapeutic agent. In such embodiments, a decrease in the activity, expression, or amount of one or more biomarker in the sample profile as compared with a control profile after contacting the subject with a cancer therapeutic agent may be indicative of higher susceptibility to treatment with an agent capable of inhibiting or reversing EMT or a chemotherapeutic agent.

In some such embodiments, an increase in the activity, expression, or amount of one or more biomarker in the sample profile as compared with a control profile after contacting the subject with a cancer therapeutic agent may be indicative of lower susceptibility to treatment with an agent capable of inhibiting or reversing EMT or a chemotherapeutic agent. In some such embodiments, a decrease in the activity, expression, or amount of one or more biomarker in the sample profile as compared with a control profile after contacting the subject with a cancer therapeutic agent may be indicative of lower susceptibility to treatment with an agent capable of inhibiting or reversing EMT or a chemotherapeutic agent.

In some preferred embodiments the one or more biomarker includes at least Axl. In some embodiments the one or more biomarker is Axl. In some embodiments an increase in the activity, expression, or amount of Axl in the sample profile as compared with a control profile after contacting the subject with a cancer therapeutic agent is indicative of higher susceptibility to treatment with an agent capable of inhibiting or reversing EMT or a chemotherapeutic agent.

Subject

A “subject” as referred to herein is any species of animal classified as a mammal, and includes but is not restricted to, domestic and farm animals, primates and humans. In some preferred embodiments the subject is a human of any sex or race. In some embodiments, the human is an adult human.

In some embodiments of this aspect of the disclosure, the subject was selected for treatment with the agent capable of inhibiting or reversing EMT or a chemotherapeutic agent, using a predictive method according to the first aspect of this facet of the disclosure outlined above.

Agents Capable of Inhibiting or Reversing EMT

The present disclosure provides methods for predicting a cancer-related outcome in a subject having, suspected of having, or diagnosed with triple-negative breast cancer (TNBC). A “cancer-related outcome” is a clinical prediction or prognosis associated with cancer.

The agents capable of inhibiting or reversing EMT for use in this TNBC facet of the disclosure are defined as described above in the AML facet (see identically titled section above on page 14, line 19 to page 17, line 2).

In some preferred embodiments the further cancer treatment is a PD-1 inhibitor or PD-L1 inhibitor. In some embodiments the further cancer treatment is a monoclonal antibody such as pembrolizumab, alemtuzumab, bevacizumab, cetuximab, ibritumomab, tiuxetan, or rituximab. In some particularly preferred embodiments the further cancer treatment is pembrolizumab.

Assessing Biomarker Activity, Expression, Amount

In some embodiments of the predictive methods described herein, assessing the activity, expression, or amount of one or more biomarker in a subject, or in a sample derived from a subject, comprises: contacting a sample, or an extract from a sample, with at least 1, 2, 3, 4, 5, 6, 7, 8, 9, 10 or more reagents for detecting the activity, expression, or amount of one or more biomarkers, each of which is selective for a particular biomarker; and, detecting and/or quantifying said reagents for detecting.

In some preferred embodiments, assessing the expression or amount of the one or more biomarker comprises determining the level of the one or more biomarker protein or mRNA in the subject or sample derived from the subject. Preferably the expression or amount of the one or more biomarker is assessed by determining the level of protein expression. In particularly preferred embodiments the expression or amount of the one or more biomarker is assessed by determining the level of protein expression in serum.

The term “expression” refers to the transcription of a gene's DNA template to produce the corresponding mRNA and translation of this mRNA to produce the corresponding gene product (i.e., a peptide, polypeptide, or protein) as well as the “expression” of a protein in one or more forms that may have been modified post translation.

In some embodiments determining the level of the one or more biomarker protein or mRNA comprises: contacting a sample, or an extract from a sample, with at least 1, 2, 3, 4, 5, 6, 7, 8, 9, 10 or more specific binding members, each of which selectively binds to a respective biomarker protein or mRNA; and, detecting and/or quantifying formation of complex formed by said specific binding member and said biomarker protein or mRNA. In some embodiments the specific binding member may comprise an antibody molecule or a binding fragment thereof.

A range of suitable techniques for determining protein and mRNA expression levels are well known in the art, for example microarray analysis, Western blotting, and PCR techniques such as QPCR. In some embodiments, determining the expression or amount of the one or more biomarker protein or mRNA may comprise use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.

In some embodiments, the expression or amount of the one or more biomarker is determined as an absolute amount of the respective biomarker protein or mRNA in the sample. In such cases, the respective biomarker protein or mRNA may be quantified in the sample and the amount expressed as a measure of the sample, for example the volume, mass, or total protein content of the sample.

In some embodiments, the expression or amount of the one or more biomarker is determined as a relative amount of the respective biomarker protein or mRNA in the sample, wherein the relative amount is determined relative to a reference protein or mRNA in the sample. Determining the relative amount of biomarker protein or mRNA in this way allows for normalisation to, e.g., account for differences in total protein concentration and to remove bias from sample to sample.

Preferably the reference protein or mRNA is one whose expression or amount does not vary significantly between subjects having cancer and subjects not having cancer, in particular TNBC. That is, the reference protein or mRNA is preferably one whose expression or amount is not altered by TNBC. Accordingly, the reference protein or mRNA may comprise one or more protein or mRNA which is not one of the biomarkers described herein. When expressing the expression or amount of the one or more biomarker as a relative amount, the absolute amount of the respective biomarker protein or mRNA in the sample may be divided by the absolute amount of the reference protein or mRNA in the sample.

Assessing Biomarker Expression at the RNA Level

Gene expression can be detected at the RNA level. RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland). Typical assay formats utilising ribonucleic acid hybridisation include nuclear run-on assays, RT-PCR, RNase protection assays (Melton et al., Nuc. Acids Res. 12:7035), Northern blotting and In Situ hybridization. Gene expression can also be detected by microarray analysis as described below.

Assessing Biomarker Expression at the Protein Level

Preferably biomarker level is assessed by measuring protein expression. Altered gene or protein expression may also be detected by measuring the polypeptides encoded by the gene. This may be achieved by using molecules which bind to the polypeptides encoded by the biomarker gene. Suitable molecules/agents which bind either directly or indirectly to the polypeptides in order to detect the presence of the protein include naturally occurring molecules such as peptides and proteins, for example antibodies, or they may be synthetic molecules.

Antibodies may be derived from commercial sources or through techniques which are familiar to those skilled in the art. In one embodiment, and where altered expression manifests itself through the expression of alteration of post translationally-modified forms of a protein biomarker, antibodies specific for those different forms may be used. For the purposes of this disclosure, the term “antibody”, unless specified to the contrary, includes whole antibodies, or fragments of whole antibodies which retain their binding activity for a target antigen. Such fragments include Fv, F(ab′) and F(ab′)2 fragments, as well as single chain antibodies (scFv). Furthermore, the antibodies and fragments thereof may be humanised antibodies, for example as described in EP239400A. For example: monoclonal and polyclonal antibodies, recombinant antibodies, proteolytic and recombinant fragments of antibodies (Fab, Fv, scFv, diabodies), single-domain antibodies (VHH, sdAb, nanobodies, IgNAR, VNAR), and proteins unrelated to antibodies, which have been engineered to have antibody-like specific binding. Antibodies may be bound to a solid support and/or packaged into kits in a suitable container along with suitable reagents, controls, instructions and the like.

Arrays

Array technology and the various techniques and applications associated with it are well known in the art. Array technology overcomes the disadvantages with traditional methods in molecular biology, which generally work on a “one gene in one experiment” basis, resulting in low throughput and the inability to appreciate the “whole picture” of gene function. In the context of the products and methods of the present disclosure, array technology can be used, for example, in the analysis of the expression of biomarker proteins or mRNA.

In general, any library or group of samples may be arranged in an orderly manner into an array, by spatially separating the members of the library or group. Examples of suitable libraries for arraying include nucleic acid libraries (including DNA, cDNA, oligonucleotide, etc. libraries), peptide, polypeptide and protein libraries, as well as libraries comprising any molecules, such as ligand libraries, among others. Accordingly, where reference is made to a “library” in this document, unless the context dictates otherwise, such reference should be taken to include reference to a library in the form of an array.

Proteins, polypeptides, etc., may also be immobilised in arrays. For example, antibodies have been used in microarray analysis of the proteome using protein chips (Borrebaeck C A, 2000, Immunol Today 21(8):379-82). Polypeptide arrays are reviewed in, for example, MacBeath and Schreiber, 2000, Science, 289(5485):1760-1763.

Sample

Suitable samples include, but are not limited to, tissue samples such as tissue biopsy, blood, urine, buccal scrapes etc., as well as serum, plasma, or tissue culture supernatant samples. In preferred embodiments the expression or amount of the one or more biomarker is assessed by determining the level of protein expression in the sample. In some preferred embodiments the sample is a blood, serum, or plasma sample. In some particularly preferred embodiments the sample is a serum sample.

When assessing the expression or amount of the one or more biomarker proteins or mRNA in serum or plasma samples derived from a subject, samples are removed and subjected to analytical techniques such as flow cytometry, mass cytometry (CyTOF), ELISA, PET, and SELDI-TOF MS. In some embodiments, the method may comprise extracting RNA from said sample and detecting gene expression by QPCR. In some other embodiments, gene expression may be detected by detecting protein products by, for example, Western Blot.

In some embodiments, the level of expression of the one or more biomarker is assessed by determining the copy number of the gene encoding the one or more biomarker. Copy number (i.e. gene duplication events) may be determined using standard techniques known in the art, e.g. using a DNA chip as described in Jiang et al. (Jiang Q, Ho Y Y, Hao L, Nichols Berrios C, Chakravarti A. Copy number variants in candidate genes are genetic modifiers of Hirschsprung disease. PLoS One. 2011; 6(6)).

Is some preferred embodiments of the predictive methods described herein, the method is performed in vitro or ex vivo.

Methods of Selecting a Subject for Treatment

An third aspect of this facet of the present disclosure relates to methods of selecting subjects having, suspected of having, or diagnosed with triple-negative breast cancer (TNBC) for treatment with an agent capable of inhibiting or reversing EMT or a chemotherapeutic agent. In some embodiments the method comprises: identifying subjects susceptible to treatment with an agent capable of inhibiting or reversing EMT or a chemotherapeutic agent using a predictive method defined in accordance with the above described first and second aspects of this facet of the disclosure; and, selecting thus identified subjects for treatment.

In some embodiments this aspect relates to methods of selecting subjects having, suspected of having, or diagnosed with triple-negative breast cancer (TNBC) for continued treatment with an agent capable of inhibiting or reversing EMT or a chemotherapeutic agent. In some embodiments the method comprises: identifying subjects susceptible to treatment with an agent capable of inhibiting or reversing EMT or a chemotherapeutic agent using a predictive method defined in accordance with the above described second aspect of this facet of the disclosure; and, selecting thus identified subjects for treatment.

In some embodiments of this aspect of the disclosure, the treatment comprises administering to the subject a therapeutically effective amount of an agent capable of inhibiting or reversing EMT or a chemotherapeutic agent as described herein. Accordingly, in some embodiments the treatment comprises an Axl inhibitor, Akt3 inhibitor, Slfn11 inhibitor, or PHGDH inhibitor. In some preferred embodiments the treatment comprises an Axl inhibitor, for example, BGB324/R428/bemcentinib, cabozantinib, TP-0903, foretinib, merestinib, bosutinib, gilteritinib, crizotinib, amuvatinib, sunitinib, sitravatinib, LDC1267, or UNC2025. Other Axl inhibitors include the anti-Axl antibodies described in WO2015/193428, WO2015/193430, WO2016/097370, and WO2016/166296. In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In some other preferred embodiments, the treatment comprises an Akt3 inhibitor. In some embodiments the Akt3 inhibitor is an Akt3 inhibitor disclosed in WO2016/102672.

In some embodiments of this aspect of the disclosure, the treatment is administered as a single agent. In other embodiments, the treatment is administered in combination with a further cancer treatment. Suitable further cancer treatments are outlined in detail above. In some preferred embodiments the further cancer treatment is a PD-1 inhibitor or PD-L1 inhibitor. In some embodiments the further cancer treatment is a monoclonal antibody such as pembrolizumab, alemtuzumab, bevacizumab, cetuximab, ibritumomab, tiuxetan, or rituximab. In some particularly preferred embodiments the further cancer treatment is pembrolizumab.

Kits

A fourth aspect of this facet of the present disclosure relates to diagnostic kits and test devices. In some embodiments the disclosure provides a diagnostic kit comprising 1, 2, 3, 4, 5, 6, 7, 8, 9, 10 or more reagents for detecting the activity, expression, or amount of one or more of the biomarkers described herein, each of which is selective for a particular biomarker. In some embodiments each of the reagents for detecting is selective for a biomarker selected from the group consisting of: Axl, IL-6R beta, ICAM-1, MIP-1 beta, Periostin, IL-23, FOLR3, CLU, and/or FasL.

In other embodiments the disclosure provides a test device comprising 1, 2, 3, 4, 5, 6, 7, 8, 9, 10 or more reagents for detecting the activity, expression, or amount of one or more of the biomarkers described herein, each of which is selective for a particular biomarker. In some embodiments each of the reagents for detecting is selective for a biomarker selected from the group consisting of: Axl, IL-6R beta, ICAM-1, MIP-1 beta, Periostin, IL-23, FOLR3, CLU, and/or FasL.

In some embodiments the diagnostic kit or test device comprises 1, 2, 3, 4, 5, 6, 7, 8, 9, 10 or more specific binding members, each of which selectively binds to one of the biomarkers described herein; and, one or more reagents for detecting said one or more specific binding members, or one or more reagents for detecting and/or quantifying formation of a complex formed by said specific binding member and said biomarker. In some embodiments each specific binding member selectively binds to a biomarker selected from the group consisting of: Axl, IL-6R beta, ICAM-1, MIP-1 beta, Periostin, IL-23, FOLR3, CLU, and/or FasL.

In some embodiments the diagnostic kit or test device comprises a specific binding member which selectively binds to Axl, and at least 1, 2, 3, 4, 5, 6, 7, 8, 9, 10, or more specific binding members, each of which selectively binds to a biomarker selected from the group consisting of: IL-6R beta, ICAM-1, MIP-1 beta, Periostin, IL-23, FOLR3, CLU, and/or FasL.

In some embodiments of this aspect of the disclosure the diagnostic kit or test device comprises a plurality of said specific binding members provided in the form of an array on a substrate or conjugated to a plurality of particles, such as beads or microspheres. The particles may be encoded with a detectable label. In some embodiments the specific binding member comprises an antibody molecule or a binding fragment thereof.

In some embodiments of the diagnostic kit or test device detecting formation of a complex formed by a specific binding member and a biomarker is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.

In some embodiments of the diagnostic kit or test device, the kit or test device comprises no more than 15, 20, 25, 30, 40, or 50 reagents for detecting. In some embodiments of the diagnostic kit or test device, the kit or test device comprises no more than 15, 20, 25, 30, 40, or 50 specific binding members.

The present disclosure also provides use of such diagnostic kits and test devices in methods of predicting a cancer-related outcome in a subject. In preferred embodiments the cancer-related outcome is susceptibility to treatment with an agent capable of inhibiting or reversing EMT or a chemotherapeutic agent as described herein.

In some embodiments the agent capable of inhibiting or reversing EMT or chemotherapeutic agent is an Axl inhibitor, Akt3 inhibitor, Slfn11 inhibitor, or PHGDH inhibitor. In some preferred embodiments the agent capable of inhibiting or reversing EMT or chemotherapeutic agent is an Axl inhibitor, for example BGB324/R428/bemcentinib, cabozantinib, TP-0903, foretinib, merestinib, bosutinib, gilteritinib, crizotinib, amuvatinib, sunitinib, sitravatinib, LDC1267, or UNC2025. Other Axl inhibitors include the anti-Axl antibodies described in WO2015/193428, WO2015/193430, WO2016/097370, and WO2016/166296. In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In some embodiments of the diagnostic kit or test device for use in a predictive method, the subject has, is suspected of having, or has been diagnosed with TNBC.

In some embodiments the method may be as defined in accordance with the above described first and second aspects of this facet of the disclosure.

A fifth aspect of the present disclosure relates to the use of a reagent for detecting the activity, expression, or amount of a biomarker selected from the group consisting of: Axl, IL-6R beta, ICAM-1, MIP-1 beta, Periostin, IL-23, FOLR3, CLU, and/or FasL, in a method of predicting a cancer-related outcome in a subject.

A sixth aspect of this facet of the disclosure relates to the use of 1, 2, 3, 4, 5, 6, 7, 8, 9, 10 or more reagents for detecting the activity, expression, or amount a biomarker, each of which is selective for a particular biomarker selected from the group consisting of: Axl, IL-6R beta, ICAM-1, MIP-1 beta, Periostin, IL-23, FOLR3, CLU, and/or FasL, in a method of manufacture of a diagnostic kit or test device for use in a method of predicting a cancer-related outcome in a subject. In some embodiments of these uses the reagent for detecting comprises a specific binding member selective for a particular biomarker. The specific binding member may comprise an antibody molecule or a binding fragment thereof.

In some embodiments the method may be as defined in accordance with the above described first and second aspects of this facet of the disclosure.

Uses

A seventh aspect of this facet of the present disclosure relates to the use of one or more of: Axl, IL-6R beta, ICAM-1, MIP-1 beta, Periostin, IL-23, FOLR3, CLU, and/or FasL, as a biomarker for determining if a subject is susceptible to treatment with an agent capable of inhibiting or reversing EMT or a chemotherapeutic agent.

In some embodiments the use relates to use of Axl, and at least 1, 2, 3, 4, 5, 6, 7, 8, 9, 10, or more of: IL-6R beta, ICAM-1, MIP-1 beta, Periostin, IL-23, FOLR3, CLU, and/or FasL, as a biomarker for determining if a subject is susceptible to treatment with an agent capable of inhibiting or reversing EMT or a chemotherapeutic agent.

In some embodiments the use is as a biomarker in a method as defined in accordance with the above described first and second aspects of this facet of the disclosure.

Methods of Treating a Subject

A eighth aspect of this facet of the present disclosure relates to methods of treating a subject having, suspected of having, or diagnosed with triple-negative breast cancer (TNBC). In some embodiments the method comprises: identifying a subject for treatment using a predictive method defined in accordance with the above described first and second aspects of this facet of the disclosure; and, administering to the subject a therapeutically effective amount of an agent capable of inhibiting or reversing EMT or a chemotherapeutic agent.

Accordingly, in some embodiments, the method comprises: (a) obtaining a sample from the subject; (b) determining if the subject is susceptible to treatment with an agent capable of inhibiting or reversing EMT or a chemotherapeutic agent using a predictive method defined in accordance with the above described first and second aspects of this facet of the disclosure; and, (c) administering to the subject a therapeutically effective amount of an agent capable of inhibiting or reversing EMT or a chemotherapeutic agent.

In some embodiments the method comprises administering a therapeutically effective amount of an agent capable of inhibiting or reversing EMT or chemotherapeutic agent to a subject identified as susceptible to said treatment using a predictive method defined in accordance with the above described first and second aspects of this facet of the disclosure.

In other embodiments the method comprises: (a) administering to a subject a therapeutically effective amount of an agent capable of inhibiting or reversing EMT or a chemotherapeutic agent; and, (b) determining if the subject is susceptible to said treatment using a predictive method defined in accordance with the above described second aspect of this facet of the disclosure. In some embodiments the method further comprises: (c) administering to the subject one or more further therapeutically effective amounts of an agent capable of inhibiting or reversing EMT or a chemotherapeutic agent provided that the subject is identified as susceptible to said treatment.

A ninth aspect of this facet of the present disclosure relates to an agent capable of inhibiting or reversing EMT or chemotherapeutic agent for use in a method of treating a subject having, suspected of having, or diagnosed with triple-negative breast cancer (TNBC). In some embodiments the method of treatment is a method as defined above.

A tenth aspect of this facet of the present disclosure relates to use of an agent capable of inhibiting or reversing EMT or chemotherapeutic agent for use in a method of manufacture of a medicament for use in a method of treating a subject having, suspected of having, or diagnosed with triple-negative breast cancer (TNBC). In some embodiments the method of treatment is a method as defined above.

In some embodiments of these methods, agents for use, or uses, the agent capable of inhibiting or reversing EMT or chemotherapeutic agent comprises an Axl inhibitor, Akt3 inhibitor, Slfn11 inhibitor, or PHGDH inhibitor. In some preferred embodiments the agent capable of inhibiting or reversing EMT or chemotherapeutic agent is an Axl inhibitor, for example BGB324/R428/bemcentinib, cabozantinib, TP-0903, foretinib, merestinib, bosutinib, gilteritinib, crizotinib, amuvatinib, sunitinib, sitravatinib, LDC1267, or UNC2025. Other Axl inhibitors include the anti-Axl antibodies described in WO2015/193428, WO2015/193430, WO2016/097370, and WO2016/166296. In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In some other preferred embodiments, the treatment comprises an Akt3 inhibitor. In some embodiments the Akt3 inhibitor is an Akt3 inhibitor disclosed in WO2016/102672.

In some embodiments of these methods, agents for use, or uses, the treatment is administered as a single agent. In other embodiments, the treatment is administered in combination with a further cancer treatment. Suitable further cancer treatments are outlined in detail above. In some preferred embodiments the further cancer treatment is a PD-1 inhibitor or PD-L1 inhibitor. In some embodiments the further cancer treatment is a monoclonal antibody such as pembrolizumab, alemtuzumab, bevacizumab, cetuximab, ibritumomab, tiuxetan, or rituximab. In some particularly preferred embodiments the further cancer treatment is pembrolizumab.

Statements of Disclosure

The following numbered statements relate to aspects of this facet of the present disclosure, and form part of the description:
101. A method of predicting a cancer-related outcome in a subject, the method comprising assessing the activity, expression, or amount of one or more biomarker in the subject, or in a sample derived from the subject;
   wherein the one or more biomarker is selected from the group consisting of: Axl, IL-6R beta, ICAM-1, MIP-1 beta, Periostin, IL-23, FOLR3, CLU, and/or FasL; and
   wherein the subject has, is suspected of having, or has been diagnosed with triple-negative breast cancer (TNBC).
102. A method according to statement 101, wherein the method comprises:
   assessing the activity, expression, or amount of one or more biomarker in the subject, or in a sample derived from the subject, to obtain a sample profile of the one or more biomarker; and
   making a prediction based on the sample profile of the one or more biomarker.
103. A method according to statement 102, wherein the prediction is made by comparing the sample profile to a control profile.
104. A method according to statement 103, wherein the sample profile is obtained before the subject is contacted with or administered an agent capable of inhibiting or reversing EMT or a chemotherapeutic agent.
105. A method according to statement 104, wherein the control profile is:
   (i) obtained from a population of control subjects having TNBC; or
   (ii) obtained from a control subject or population of control subjects having TNBC and previously found to lack susceptibility to treatment with an agent capable of inhibiting or reversing EMT or a chemotherapeutic agent (that is, non-responder subjects);
   (iii) a predetermined profile of biomarker expression, activity, or amount values, for example a profile of “average, median, or mean” values or “standard ranges” of values, obtained from a control subject or population of control subjects having TNBC and previously found to lack susceptibility to treatment with an agent capable of inhibiting or reversing EMT or a chemotherapeutic agent (that is, non-responder subjects);
   (iv) obtained from a control sample having a known “average, median, or mean” value of biomarkers indicative of a control subject or population of control subjects having TNBC and previously found to lack susceptibility to treatment with an agent capable of inhibiting or reversing EMT or a chemotherapeutic agent (that is, non-responder subjects);
   (v) a predetermined profile of biomarker expression, activity, or amount “threshold” values obtained from a control subject or population of control subjects having TNBC and previously found to be susceptible to treatment with an agent capable of inhibiting or reversing EMT or a chemotherapeutic agent (that is, responder subjects); or
   (vi) obtained from a control sample having a known “threshold” value of biomarkers indicative of a control subject or population of control subjects having TNBC and previously found to be susceptible to treatment with an agent capable of inhibiting or reversing EMT or a chemotherapeutic agent (that is, responder subjects).
106. A method according to statement 105, wherein a higher activity, expression, or amount of one or more biomarker in the sample profile as compared with the control profile is indicative of susceptibility to treatment with an agent capable of inhibiting or reversing EMT or a chemotherapeutic agent.
107. A method according to statement 105, wherein a lower activity, expression, or amount of one or more biomarker in the sample profile as compared with the control profile is indicative of susceptibility to treatment with an agent capable of inhibiting or reversing EMT or a chemotherapeutic agent.
108. A method according to any one of statements 105-107, wherein the one or more biomarker includes at least one of: IL-23, FOLR3, CLU, and/or FasL,
   and wherein a higher activity, expression, or amount of one or more of said biomarkers in the sample profile as compared with the control profile is indicative of susceptibility to treatment with an agent capable of inhibiting or reversing EMT or a chemotherapeutic agent.
109. A method according to any one of statements 105-108, wherein the one or more biomarker includes at least one of: Axl, IL-6R beta, ICAM-1, MIP-1 beta, and/or Periostin,
   and wherein a lower activity, expression, or amount of one or more of said biomarkers in the sample profile as compared with the control profile is indicative of susceptibility to treatment with an agent capable of inhibiting or reversing EMT or a chemotherapeutic agent.
110. A method according to any one of statements 105-109, wherein:
   a lower activity, expression, or amount of Axl; and/or
   a lower activity, expression, or amount of IL-6R beta; and/or
   a lower activity, expression, or amount of ICAM-1; and/or
   a lower activity, expression, or amount of MIP-1 beta; and/or
   a lower activity, expression, or amount of Periostin; and/or
   a higher activity, expression, or amount of IL-23; and/or
   a higher activity, expression, or amount of FOLR3; and/or
   a higher activity, expression, or amount of CLU; and/or
   a higher activity, expression, or amount of FasL;
   in the sample profile as compared with the control profile is indicative of susceptibility to treatment with an agent capable of inhibiting or reversing EMT or a chemotherapeutic agent.
111. A method according to any one of statements 101-110, wherein the one or more biomarker comprises Axl,
   optionally wherein the one or more biomarker comprises Axl and at least 1, 2, 3, 4, 5, 6, 7, 8, 9, 10, or more further biomarkers selected from: IL-6R beta, ICAM-1, MIP-1 beta, Periostin, IL-23, FOLR3, CLU, and/or FasL.
112. A method according to any one of statements 101-111, wherein:
   said Axl biomarker comprises an amino acid sequence having at least 70%, 80%, 90%, 95%, 99% or 100% sequence identity with the full-length of the amino acid sequence disclosed at UniProt accession no: P30530-1 or P30530-2 (entry version 196) or a fragment thereof;
   said Interleukin-6 receptor subunit beta (IL-6R beta) biomarker comprises an amino acid sequence having at least 70%, 80%, 90%, 95%, 99% or 100% sequence identity with the full-length of the amino acid sequence disclosed at UniProt accession no: P40189-1 or P40189-2 (entry version 209) or a fragment thereof;
   said Intercellular Adhesion Molecule 1 (ICAM-1) biomarker comprises an amino acid sequence having at least 70%, 80%, 90%, 95%, 99% or 100% sequence identity with the full-length of the amino acid sequence disclosed at UniProt accession no: P05362 (entry version 221) or a fragment thereof;
   said Macrophage Inflammatory Protein-1 beta (MIP-1 beta) biomarker comprises an amino acid sequence having at least 70%, 80%, 90%, 95%, 99% or 100% sequence identity with the full-length of the amino acid sequence disclosed at UniProt accession no: P13236 (entry version 187) or a fragment thereof;
   said Periostin biomarker comprises an amino acid sequence having at least 70%, 80%, 90%, 95%, 99% or 100% sequence identity with the full-length of the amino acid sequence disclosed at UniProt accession no: Q15063-1, Q15063-2, Q15063-3, Q15063-4, Q15063-5, Q15063-6, Q15063-7 (entry version 160) or a fragment thereof;
   said Interleukin-23 (IL-23) biomarker comprises an amino acid sequence having at least 70%, 80%, 90%, 95%, 99% or 100% sequence identity with the full-length of the amino acid sequence disclosed at UniProt accession no: Q9NPF7 (entry version 133) or a fragment thereof;
   said Folate receptor gamma (FOLR3) biomarker comprises an amino acid sequence having at least 70%, 80%, 90%, 95%, 99% or 100% sequence identity with the full-length of the amino acid sequence disclosed at UniProt accession no: P41439-1, P41439-2, or P41439-3 (entry version 127) or a fragment thereof;
   said Clusterin (CLU) biomarker comprises an amino acid sequence having at least 70%, 80%, 90%, 95%, 99% or 100% sequence identity with the full-length of the amino acid sequence disclosed at UniProt accession no: P10909-1, P10909-2, P10909-3, P10909-4, or P10909-5 (entry version 209) or a fragment thereof; and/or
   said Fas Ligand (FasL) biomarker comprises an amino acid sequence having at least 70%, 80%, 90%, 95%, 99% or 100% sequence identity with the full-length of the amino acid sequence disclosed at UniProt accession no: P48023-1 or P48023-2 (entry version 197) or a fragment thereof;
   or a nucleic acid (either DNA or RNA) having at least 70%, 80%, 90%, 95%, 99% or 100% sequence identity with the full-length nucleic acid sequence encoding said amino acid sequences.
113. A method according to any one of statement 101-112, wherein the method comprises determining the amount of at least 2, 3, 4, 5, 6, 7, 8, 9, 10, 11, 12, 13, 14, 15, 20 of said biomarkers in the subject, or sample derived from the subject.
114. A method according to any one of statements 104-113, wherein assessing the activity, expression, or amount of one or more biomarker in the subject, or in a sample derived from the subject, comprises:
   contacting a sample, or an extract from the sample, with at least 1, 2, 3, 4, 5, 6, 7, 8, 9, 10 or more reagents for detecting the activity, expression, or amount of one or more biomarkers, each of which is selective for a particular biomarker; and
   detecting and/or quantifying said reagents for detecting.
115. A method according to statement 114, wherein assessing the expression or amount of one or more biomarker in the subject, or in a sample derived from the subject, comprises:
   contacting a sample, or an extract from the sample, with at least 1, 2, 3, 4, 5, 6, 7, 8, 9, 10 or more specific binding members, each of which selectively binds to a respective biomarker protein or mRNA; and
   detecting and/or quantifying formation of complex formed by said specific binding member and said biomarker protein or mRNA.
116. A method according to statement 115 wherein the specific binding member comprises an antibody molecule or binding fragment thereof.
117. A method according to statement 115 or 116, wherein detecting formation of a complex formed by the specific binding member and the biomarker protein or mRNA is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.
118. A method according to any one of statements 115-117 wherein the expression or amount of the one or more biomarker is determined as the absolute amount of the respective biomarker protein or mRNA in the sample.
119. A method according to any one of statements 115-117 wherein the expression or amount of the one or more biomarker is determined as a relative amount of the respective biomarker protein or mRNA in the sample,
   optionally wherein the relative amount is determined relative to a reference protein or mRNA in the sample, the expression of which is not altered by TNBC.
120. A method according to any one of statements 101-119, wherein the cancer-related outcome is susceptibility to treatment with an agent capable of inhibiting or reversing EMT or a chemotherapeutic agent.
121. A method according to statement 120, wherein the agent capable of inhibiting or reversing EMT or chemotherapeutic agent is an Axl inhibitor, Akt3 inhibitor, Slfn11 inhibitor, or PHGDH inhibitor.
122. A method according to statement 121, wherein the agent capable of inhibiting or reversing EMT or chemotherapeutic agent is an Axl inhibitor.
123. A method according to statement 122, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
124. A method according to statement 123, wherein the Axl inhibitor is BGB324/R428/bemcentinib.
125. A method according to statement 123, wherein the Axl inhibitor is an anti-Axl antibody described in WO2015/193428, WO2015/193430, WO2016/097370, or WO2016/166296.
126. A method according to statement 121, wherein the agent capable of inhibiting or reversing EMT or chemotherapeutic agent is an Akt3 inhibitor.
127. A method according to statement 126, wherein the Akt3 inhibitor is an Akt3 inhibitor disclosed in WO2016/102672.
128. A method according to any one of statements 120-127, wherein the agent capable of inhibiting or reversing EMT is administered as a single agent.
129. A method according to any one of statements 120-127, wherein the agent capable of inhibiting or reversing EMT is administered in combination with a further cancer treatment.
130. A method according to statement 129, wherein the further cancer treatment is selected from:
   (i) alkylating agents, including alkyl sulfonates such as busulfan;
   (ii) nitrogen mustards such as chlorambucil, cyclophosphamide, estramustine, ifosfamide, mechlorethamine, melphalan, and uramustine;
   (iii) ethyleneimine derivatives such as thiotepa;
   (iv) nitrosoureas such as carmustine, lomustine, and streptozocin;
   (v) triazenes such as dacarbazine, procarbazine, and temozolamide;
   (vi) platinum compounds such as cisplatin, carboplatin, oxaliplatin, satraplatin, and picoplatin onnaplatin, tetraplatin, sprioplatin, iproplatin, chloro(diethylenediamino)-platinum (II) chloride, dichloro(ethylenediamino)-platinum (II), diamino(2-ethylmalonato)platinum (II), (1,2-diaminocyclohexane)malonatoplatinum (II), (4-carboxyphthalo)-(1,2-diaminocyclohexane)platinum (II), (1,2-diaminocyclohexane)-(isocitrato)platinum (II), and (1,2-diaminocyclohexane)-cis-(pyruvato)platinum (II);
   (vii) antimetabolites, including antifolates such as methotrexate, permetrexed, raltitrexed, and trimetrexate;
   (viii) pyrimidine analogues such as azacitidine, capecitabine, cytarabine, edatrexate, floxuridine, fluorouracil, gemcitabine, and troxacitabine;
   (ix) purine analogues such as cladribine, chlorodeoxyadenosine, clofarabine, fludarabine, mercaptopurine, pentostatin, and thioguanine;
   (x) natural products, including antitumor antibiotics such as bleomycin, dactinomycin, mithramycin, mitomycin, mitoxantrone, porfiromycin;
   (xi) anthracyclines such as daunorubicin, doxorubicin, epirubicin, idarubicin, and valrubicin;
   (xii) mitotic inhibitors such as the vinca alkaloids vinblastine, vinvesir, vincristine, vindesine, and vinorelbine;
   (xiii) enzymes such as L-asparaginase and PEG-L-asparaginase;
   (xiv) microtubule polymer stabilizers such as the taxanes paclitaxel and docetaxel;
   (xv) topoisomerase I inhibitors such as the camptothecins irinotecan and topotecan;
   (xvi) topoisomerase II inhibitors such as podophyllotoxin, amsacrine, etoposide, teniposide, losoxantrone and actinomycin;
   (xvii) hormones and hormone antagonists, including
      androgens such as fluoxymesterone and testolactone;
      antiandrogens such as bicalutamide, cyproterone, flutamide, and nilutamide;
      corticosteroids such as dexamethasone and prednisone;
      aromatase inhibitors such as aminoglutethimide, anastrozole, exemestane, formestane, and letrozole;
      estrogens such as diethylstilbestrol;
      antiestrogens such as fulvestrant, raloxifene, tamoxifen, and toremifine;
      luteinising hormone-releasing hormone (LHRH) agonists and antagonists such as abarelix, buserelin, goserelin, leuprolide, histrelin, desorelin, nafarelin acetate and triptorelin;
      progestins such as medroxyprogesterone acetate and megestrol acetate; and
      thyroid hormones such as levothyroxine and liothyronine;
   (xviii) PKB pathway inhibitors, including perifosine, enzastaurin hydrochloride, and triciribine;
   (xix) P13K inhibitors such as semaphore and SF1126, and MTOR inhibitors such as rapamycin and analogues;
   (xx) CDK inhibitors, including seliciclib, alvocidib, and 7-hydroxystaurosporine;
   (xxi) COX-2 inhibitors, including celecoxib;
   (xxii) HDAC inhibitiors, including trichostatin A, suberoylanilide hydroxamic acid, and chlamydocin;
   (xxiii) DNA methylase inhibitors, including temozolomide, and miscellaneous agents, including altretamine, arsenic trioxide, thalidomide, lenalidomide, gallium nitrate, levamisole, mitotane, hydroxyurea, octreotide, procarbazine, suramin, photodynamic compounds such as methoxsalen and sodium porfimer;
   (xxiv) proteasome inhibitors such as bortezomib;
   (xxv) molecular targeted therapy agents including: functional therapeutic agents, including gene therapy agents and antisense therapy agents;
   (xxvi) tyrosine kinase inhibitors such as erlotinib hydrochloride, gefitinib, imatinib mesylate, and semaxanib;
   (xxvii) Raf inhibitors such as sorafenib, and gene expression modulators such as the retinoids and rexinoids, for example adapalene, bexarotene, trans-retinoic acid, 9-cis-retinoic acid, and N-(4-hydroxyphenyl)retinamide;
   (xxviii) phenotype-directed therapy agents, including monoclonal antibodies such as pembrolizumab, alemtuzumab, bevacizumab, cetuximab, ibritumomab tiuxetan, rituximab, and trastuzumab, immunotoxins such as gemtuzumab ozogamicin, radioimmunoconjugates such as I-tositumobab;
   (xxix) cancer vaccines;
   (xxx) biologic therapy agents including: interferons such as interferon-[alpha]2a and interferon-[alpha]2b, and interleukins such as aldesleukin, denileukin diftitox, and oprelvekin; (xxxi) anticancer therapies involving the use of protective or adjunctive agents, including: cytoprotective agents such as amifostine, and dexrazoxane, phosphonates such as pamidronate and zoledronic acid, and stimulating factors such as epoetin, darbeopetin, filgrastim, PEG-filgrastim, and sargramostim;
   (xxxii) Axl inhibitors such as 1-(6,7-dihydro-5H-benzo[6,7]cyclohepta[1,2-c]pyridazin-3-yl)-N3-((7-(S)-pyrrolidin-1-yl)-6,7,8,9-tetrahydro-5H-benzo[7]annulene-2-yl)-1H-1,2,4-triazole-3,5-diamine; and/or
   (xxxiii) further combination chemotherapeutic regimens, such as combinations of carboplatin/paclitaxel, capecitabine/docetaxel, fluorauracil/levamisole, fluorauracil/leucovorin, methotrexate/leucovorin, and trastuzumab/paclitaxel, alone or in further combination with carboplatin.
131. A method according to statement 129, wherein the further cancer treatment is a PD-1 inhibitor or PD-L1 inhibitor.
132. A method according to statement 129, wherein the further cancer treatment is a monoclonal antibody such as pembrolizumab, alemtuzumab, bevacizumab, cetuximab, ibritumomab, tiuxetan, or rituximab.
133. A method according to statement 131 or 132, wherein the further cancer treatment is pembrolizumab.
134. A method according to any one of statements 101-133, wherein the subject is mammalian.
135. A method according to statement 134, wherein the subject is human.
136. A method according to any one of statements 101-135, wherein the predictive method is performed before the subject receives a treatment or course of treatment for TNBC.
137. A method according to any one of statement 101-136, wherein the predictive method is performed during or after a treatment or course of treatment for TNBC.
138. A method according to any one of statements 101-135 or 137, wherein the subject has previously been treated with an agent capable of inhibiting or reversing EMT or a chemotherapeutic agent.
139. A method according to statement 138, wherein the subject has previously been treated with an agent selected from: a PHGDH inhibitor, a Slfn11 inhibitor, an Axl inhibitor, or an Akt3 inhibitor.
140. A method according to any one of statements 138-139, wherein the subject has previously been treated with an agent that is different to the agent capable of inhibiting or reversing EMT or chemotherapeutic agent for which the subject's susceptibility is being predicted.
141. A method according to any one of statements 101-136, wherein the subject has not previously been treated with an agent capable of inhibiting or reversing EMT or a chemotherapeutic agent.
142. A method according to statement 141, wherein the subject has not previously been treated with an Axl inhibitor.
143. A method according to statement 141, wherein the subject has not previously been treated with an Akt3 inhibitor.
144. A method according to any one of statements 101-143, wherein the sample is selected from: blood, serum, plasma, tissue biopsy, culture supernatant, or urine.
145. A method according to statement 144, wherein the sample is a blood, serum, or plasma sample.
146. A method according to statement 145, wherein the sample is a serum sample.
147. A method according to any one of statements 104-146, wherein the method further comprises a subsequent step of assessing the activity, expression, or amount of one or more biomarker in the subject, in which a sample profile is obtained after contacting the subject with an agent capable of inhibiting or reversing EMT or a chemotherapeutic agent.
148. A method according to statement 103, wherein the sample profile is obtained after contacting the subject with an agent capable of inhibiting or reversing EMT or a chemotherapeutic agent.
149. A method according to statement 147 or 148, wherein the control profile is obtained from the same subject prior to contacting said subject with the agent capable of inhibiting or reversing EMT or a chemotherapeutic agent.
150. A method according to statement 149, wherein an increase in the activity, expression, or amount of one or more biomarker in the sample profile as compared with the control profile is indicative of higher susceptibility to treatment with an agent capable of inhibiting or reversing EMT or a chemotherapeutic agent.
151. A method according to statement 149, wherein a decrease in the activity, expression, or amount of one or more biomarker in the sample profile as compared with the control profile is indicative of higher susceptibility to treatment with an agent capable of inhibiting or reversing EMT or a chemotherapeutic agent.
152. A method according to statement 149, wherein an increase in the activity, expression, or amount of one or more biomarker in the sample profile as compared with the control profile is indicative of lower susceptibility to treatment with an agent capable of inhibiting or reversing EMT or a chemotherapeutic agent.
153. A method according to statement 149, wherein a decrease in the activity, expression, or amount of one or more biomarker in the sample profile as compared with the control profile is indicative of lower susceptibility to treatment with an agent capable of inhibiting or reversing EMT or a chemotherapeutic agent.
154. A method according to statement 150, wherein the one or more biomarker includes at least Axl.
155. A method according to any one of statements 147-154, wherein assessing the activity, expression, or amount of one or more biomarker in the subject, or in a sample derived from the subject, comprises:
   contacting a sample, or an extract from the sample, with at least 1, 2, 3, 4, 5, 6, 7, 8, 9, 10 or more reagents for detecting the activity, expression, or amount of one or more biomarkers, each of which is selective for a particular biomarker; and
   detecting and/or quantifying said reagents for detecting.
156. A method according to statement 155, wherein assessing the expression or amount of one or more biomarker in the subject, or in a sample derived from the subject, comprises:
   contacting a sample, or an extract from the sample, with at least 1, 2, 3, 4, 5, 6, 7, 8, 9, 10 or more specific binding members, each of which selectively binds to a respective biomarker protein or mRNA; and
   detecting and/or quantifying formation of a complex formed by said specific binding member and said biomarker protein or mRNA.
157. A method according to statement 156, wherein the specific binding member comprises an antibody molecule or binding fragment thereof.
158. A method according to statement 156 or 157, wherein detecting formation of a complex formed by the specific binding member and the biomarker protein or mRNA is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.
159. A method according to any one of statements 156-158 wherein the expression or amount of the one or more biomarker is determined as the absolute amount of the respective biomarker protein or mRNA in the sample.
160. A method according to any one of statements 156-158 wherein the expression or amount of the one or more biomarker is determined as a relative amount of the respective biomarker protein or mRNA in the sample,
   optionally wherein the relative amount is determined relative to a reference protein or mRNA in the sample, the expression of which is not altered by TNBC.
161. A method according to any one of statements 148-160, wherein the agent capable of inhibiting or reversing EMT or chemotherapeutic agent is an Axl inhibitor, Akt3 inhibitor, Slfn11 inhibitor, or PHGDH inhibitor.
162. A method according to statement 161, wherein the agent capable of inhibiting or reversing EMT or chemotherapeutic agent is an Axl inhibitor.
163. A method according to statement 162, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
164. A method according to statement 163, wherein the Axl inhibitor is BGB324/R428/bemcentinib.
165. A method according to statement 163, wherein the Axl inhibitor is an anti-Axl antibody described in WO2015/193428, WO2015/193430, WO2016/097370, or WO2016/166296.
166. A method according to statement 161, wherein the agent capable of inhibiting or reversing EMT or chemotherapeutic agent is an Akt3 inhibitor.
167. A method according to statement 166, wherein the Akt3 inhibitor is an Akt3 inhibitor disclosed in WO2016/102672.
168. A method according to any one of statements 161-167, wherein the agent capable of inhibiting or reversing EMT is administered as a single agent.
169. A method according to any one of statements 161-167, wherein the agent capable of inhibiting or reversing EMT is administered in combination with a further cancer treatment.
170. A method according to statement 169, wherein the further cancer treatment is selected from the further cancer treatments listed in statement 130.
171. A method according to statement 169, wherein the further cancer treatment is a PD-1 inhibitor or PD-L1 inhibitor.
172. A method according to statement 169, wherein the further cancer treatment is a monoclonal antibody such as pembrolizumab, alemtuzumab, bevacizumab, cetuximab, ibritumomab, tiuxetan, or rituximab.
173. A method according to statement 171 or 172, wherein the further cancer treatment is pembrolizumab.
174. A method according to any one of statements 148-173, wherein the subject is mammalian.
175. A method according to statement 174, wherein the subject is human.
176. A method according to any one of statements 148-175, wherein the subject was selected for treatment with the agent capable of inhibiting or reversing EMT or a chemotherapeutic agent, using a predictive method according to any one of statements 101-147.
177. A method according to any one of statements 148-176, wherein the sample is selected from: blood, serum, plasma, tissue biopsy, culture supernatant, or urine.
178. A method according to statement 177, wherein the sample is a blood, serum, or plasma sample.
179. A method according to statement 178, wherein the sample is a serum sample.
180. A method according to any one of statements 1-179, wherein the method is performed in vitro or ex vivo.
201. A method of selecting subjects having, suspected of having, or diagnosed with triple-negative breast cancer (TN BC) for treatment with an agent capable of inhibiting or reversing EMT or a chemotherapeutic agent, the method comprising:
   identifying subjects susceptible to treatment with an agent capable of inhibiting or reversing EMT or a chemotherapeutic agent using a method according to any one of statements 101-180; and
   selecting thus identified subjects for treatment.
202. A method of selecting subjects having, suspected of having, or diagnosed with triple-negative breast cancer (TNBC) for continued treatment with an agent capable of inhibiting or reversing EMT or a chemotherapeutic agent, the method comprising:
   identifying subjects susceptible to treatment with an agent capable of inhibiting or reversing EMT or a chemotherapeutic agent using a method according to any one of statements 148-180; and
   selecting thus identified subjects for treatment.
203. A method according to any one of statements 201-202, wherein treatment comprises administering to the subject a therapeutically effective amount of an agent capable of inhibiting or reversing EMT or a chemotherapeutic agent.
204. A method according to statement 203, wherein the treatment comprises an Axl inhibitor, Akt3 inhibitor, Slfn11 inhibitor, or PHGDH inhibitor.
205. A method according to statement 204, wherein the treatment comprises an Axl inhibitor.
206. A method according to statement 205, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
207. A method according to statement 206, wherein the Axl inhibitor is BGB324/R428/bemcentinib.
208. A method according to statement 206, wherein the Axl inhibitor is an anti-Axl antibody described in WO2015/193428, WO2015/193430, WO2016/097370, or WO2016/166296.
209. A method according to statement 204, wherein the treatment comprises an Akt3 inhibitor.
210. A method according to statement 209, wherein the Akt3 inhibitor is an Akt3 inhibitor disclosed in WO2016/102672.
211. A method according to any one of statements 203-210, wherein the treatment is administered as a single agent.
212. A method according to any one of statements 203-211, wherein the treatment is administered in combination with a further cancer treatment.
213. A method according to statement 212, wherein the further cancer treatment is selected from the further cancer treatments listed in statement 130.
214. A method according to statement 212, wherein the further cancer treatment is a PD-1 inhibitor or PD-L1 inhibitor.
215. A method according to statement 212, wherein the further cancer treatment is a monoclonal antibody such as pembrolizumab, alemtuzumab, bevacizumab, cetuximab, ibritumomab, tiuxetan, or rituximab.
216. A method according to statement 214 or 215, wherein the further cancer treatment is pembrolizumab.
301. A diagnostic kit comprising 1, 2, 3, 4, 5, 6, 7, 8, 9, 10 or more reagents for detecting the activity, expression, or amount of one or more biomarkers, each of which is selective for a particular biomarker selected from the group consisting of: Axl, IL-6R beta, ICAM-1, MIP-1 beta, Periostin, IL-23, FOLR3, CLU, and/or FasL.
302. A test device comprising 1, 2, 3, 4, 5, 6, 7, 8, 9, 10 or more reagents for detecting the activity, expression, or amount of one or more biomarkers, each of which is selective for a particular biomarker selected from the group consisting of: Axl, IL-6R beta, ICAM-1, MIP-1 beta, Periostin, IL-23, FOLR3, CLU, and/or FasL.
303. A diagnostic kit or test device according to any one of statements 301 or 302, comprising:
   1, 2, 3, 4, 5, 6, 7, 8, 9, 10 or more specific binding members, each of which selectively binds to a biomarker selected from the group consisting of: Axl, IL-6R beta, ICAM-1, MIP-1 beta, Periostin, IL-23, FOLR3, CLU, and/or FasL; and
   one or more reagents for detecting said one or more specific binding members or one or more reagents for detecting and/or quantifying formation of a complex formed by said specific binding member and said biomarker.
304. A diagnostic kit or test device according to statement 303, wherein the specific binding member comprises an antibody molecule or binding fragment thereof.
305. A diagnostic kit or test device according to statement 303 or 304, wherein detecting formation of a complex formed by the specific binding member and the biomarker is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.
306. A diagnostic kit or test device according to any one of statements 301-305, wherein the kit or test device comprises no more than 15, 20, 25, 30, 40, or 50 reagents for detecting.
307. A diagnostic kit or test device according to any one of statements 301-306 for use in a method of predicting a cancer-related outcome in a subject.
308. A diagnostic kit for use, or test device for use, according to statement 307, wherein the cancer-related outcome is susceptibility to treatment with an agent capable of inhibiting or reversing EMT or a chemotherapeutic agent.
309. A diagnostic kit for use, or test device for use, according to statement 308, wherein the agent capable of inhibiting or reversing EMT or chemotherapeutic agent is an Axl inhibitor, Akt3 inhibitor, Slfn11 inhibitor, or PHGDH inhibitor.
310. A diagnostic kit for use, or test device for use, according to statement 308, wherein the agent capable of inhibiting or reversing EMT or chemotherapeutic agent is an Axl inhibitor.
311. A diagnostic kit for use, or test device for use, according to statement 310, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
312. A diagnostic kit for use, or test device for use, according to statement 311, wherein the Axl inhibitor is BGB324/R428/bemcentinib.
313. A diagnostic kit for use, or test device for use, according to statement 311, wherein the Axl inhibitor is an anti-Axl antibody described in WO2015/193428, WO2015/193430, WO2016/097370, or WO2016/166296.
314. A diagnostic kit for use, or test device for use, according to statement 308, wherein the agent capable of inhibiting or reversing EMT or chemotherapeutic agent is an Akt3 inhibitor.
315. A diagnostic kit for use, or test device for use, according to any one of statements 307-310, wherein the subject has, is suspected of having, or has been diagnosed with triple-negative breast cancer (TNBC).
316. A diagnostic kit for use, or test device for use, according to any one of statements 307-315, wherein the method is a method according to any one of statements 101-180.
317. Use of a reagent for detecting the activity, expression, or amount of a biomarker selected from the group consisting of: Axl, IL-6R beta, ICAM-1, MIP-1 beta, Periostin, IL-23, FOLR3, CLU, and/or FasL, in a method of predicting a cancer-related outcome in a subject.
318. Use of 1, 2, 3, 4, 5, 6, 7, 8, 9, 10 or more reagents for detecting the activity, expression, or amount a biomarker, each of which is selective for a particular biomarker selected from the group consisting of: Axl, IL-6R beta, ICAM-1, MIP-1 beta, Periostin, IL-23, FOLR3, CLU, and/or FasL, in a method of manufacture of a diagnostic kit or test device for use in a method of predicting a cancer-related outcome in a subject.
319. A use according to statement 317 or 318, wherein the reagent for detecting comprises a specific binding member selective for a particular biomarker.
320. A use according to statement 317 or 318, wherein the method of predicting a cancer-related outcome in a subject is a method according to any one of statements 101-180.
401. Use of one or more of: Axl, IL-6R beta, ICAM-1, MIP-1 beta, Periostin, IL-23, FOLR3, CLU, and/or FasL, as a biomarker for determining if a subject is susceptible to treatment with an agent capable of inhibiting or reversing EMT or a chemotherapeutic agent.
402. A use according to statement 401, wherein:
   (i) a higher activity, expression, or amount of one or more of said biomarkers in the subject, or sample derived from the subject, compared with a control profile for the respective biomarker is indicative of susceptibility to treatment with an agent capable of inhibiting or reversing EMT or a chemotherapeutic agent; or
   (ii) a lower activity, expression, or amount of one or more of said biomarkers in the subject, or sample derived from the subject, compared with a control profile for the respective biomarker is indicative of susceptibility to treatment with an agent capable of inhibiting or reversing EMT or a chemotherapeutic agent.
403. A use according to statement 401, wherein:
   (i) an increase in the activity, expression, or amount of one or more biomarker relative to a control profile for the respective biomarker after contacting the subject with a cancer therapeutic agent is indicative of increased susceptibility to treatment with an agent capable of inhibiting or reversing EMT or a chemotherapeutic agent; or
   (ii) a decrease in the activity, expression, or amount of one or more biomarker relative to a control profile for the respective biomarker after contacting the subject with a cancer therapeutic agent is indicative of increased susceptibility to treatment with an agent capable of inhibiting or reversing EMT or a chemotherapeutic agent.
404. A use according to any one of statements 401-403, wherein the use is as a biomarker in a method according to any one of statements 101-180
   501. A method of treating a subject having, suspected of having, or diagnosed with triple-negative breast cancer (TNBC), the method comprising:
   identifying a subject susceptible to treatment using a method according to any one of statements 101-180; and
   administering to the subject a therapeutically effective amount of an agent capable of inhibiting or reversing EMT or a chemotherapeutic agent.
502. A method of treating a subject having, suspected of having, or diagnosed with triple-negative breast cancer (TNBC), the method comprising:
   (a) obtaining a sample from the subject;
   (b) determining if the subject is susceptible to treatment with an agent capable of inhibiting or reversing EMT or a chemotherapeutic agent using a method according to any one of statements 101-180;
   (c) administering to the subject a therapeutically effective amount of an agent capable of inhibiting or reversing EMT or a chemotherapeutic agent.
503. A method of treating a subject having, suspected of having, or diagnosed with triple-negative breast cancer (TNBC), the method comprising administering a therapeutically effective amount of an agent capable of inhibiting or reversing EMT or chemotherapeutic agent to a subject identified as susceptible to said treatment using a method according to any one of statements 101-180.
504. A method of treating a subject having, suspected of having, or diagnosed with triple-negative breast cancer (TNBC), the method comprising:
   administering to the subject a therapeutically effective amount of an agent capable of inhibiting or reversing EMT or a chemotherapeutic agent; and
   determining if the subject is susceptible to said treatment using a method according to any one of statements 148-180.
505. A method according to statement 504, wherein the method comprises administering to the subject one or more further therapeutically effective amounts of an agent capable of inhibiting or reversing EMT or a chemotherapeutic agent provided that the subject is identified as susceptible to said treatment.
506. An agent capable of inhibiting or reversing EMT or chemotherapeutic agent for use in a method of treating a subject having, suspected of having, or diagnosed with triple-negative breast cancer (TNBC), wherein the method is a method according to any one of statements 501-505.
507. Use of an agent capable of inhibiting or reversing EMT or chemotherapeutic agent for use in a method of manufacture of a medicament for use in a method of treating a subject having, suspected of having, or diagnosed with triple-negative breast cancer (TNBC), wherein the method of treatment is a method according to any one of statements 501-505.
508. A method, agent for use, or use according to any one of statements 501-507, wherein the agent capable of inhibiting or reversing EMT or chemotherapeutic agent comprises an Axl inhibitor, Akt3 inhibitor, Slfn11 inhibitor, or PHGDH inhibitor.
509. A method, agent for use, or use according to statement 508, wherein the agent capable of inhibiting or reversing EMT or chemotherapeutic agent comprises an Axl inhibitor.
510. A method, agent for use, or use according to statement 509, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
511. A method, agent for use, or use according to statement 510, wherein the Axl inhibitor is BGB324/R428/bemcentinib.
512. A method, agent for use, or use according to statement 510, wherein the Axl inhibitor is an anti-Axl antibody described in WO2015/193428, WO2015/193430, WO2016/097370, or WO2016/166296.
512. A method, agent for use, or use according to statement 508, wherein the agent capable of inhibiting or reversing EMT or chemotherapeutic agent comprises an Akt3 inhibitor.
513. A method, agent for use, or use according to statement 512, wherein the Akt3 inhibitor is an Akt3 inhibitor disclosed in WO2016/102672.
514. A method, agent for use, or use according to any one of statements 501-513, wherein the agent capable of inhibiting or reversing EMT or chemotherapeutic agent is administered as a single agent.
515. A method, agent for use, or use according to any one of statements 501-513, wherein the agent capable of inhibiting or reversing EMT or chemotherapeutic agent is administered in combination with a further cancer treatment.
516. A method, agent for use, or use according to statement 515, wherein the further cancer treatment is selected from the further cancer treatments listed in statement 130.
517. A method, agent for use, or use according to statement 515, wherein the further cancer treatment is a PD-1 inhibitor or PD-L1 inhibitor.
518. A method, agent for use, or use according to statement 515, wherein the further cancer treatment is a monoclonal antibody such as pembrolizumab, alemtuzumab, bevacizumab, cetuximab, ibritumomab, tiuxetan, or rituximab.
519. A method, agent for use, or use according to statement 517 or 518, wherein the further cancer treatment is pembrolizumab.

Facet 6: Non-Small Cell Lung Cancer (NSCLC)

Non-small cell lung cancers (NSCLC) are any type of epithelial lung cancer other than small cell lung carcinoma (SCLC), and make up the majority of all lung cancers. Non-small cell lung carcinomas are grouped together because their prognosis and management are similar—the three main histologic classes are: squamous cell lung carcinoma, adenocarcinoma, and large cell lung carcinoma. Adenocarcinoma is the most common histologic type of lung carcinoma. Non-small cell lung cancers are often treated with cisplatin or carboplatin, in combination with gemcitabine, paclitaxel, docetaxel, etoposide or vinorelbine. There remains a need for targeted therapies for NSCLC (Herbst et al, 2018; Zappa et al, 2016).

Accordingly, robust biomarkers for predicting cancer-related outcomes in a subject having NSCLC, for example susceptibility to treatment with an agent capable of inhibiting or reversing EMT or a chemotherapeutic agent, would be useful in identifying subjects who are most likely to respond to or benefit from treatment with a particular agent/regimen.

A “responder” subject as described herein is a subject who is likely to, or who does, respond to treatment (or benefit from treatment) with an agent capable of inhibiting or reversing EMT or a chemotherapeutic agent, for example the small molecule Axl kinase inhibitor BGB324/R428/bemcentinib when administered either as a single agent or in combination with another cancer treatment.

Correspondingly, a “non-responder” subject as described herein is a subject who is not likely to, or who does not, respond to treatment (or benefit from treatment) with an agent capable of inhibiting or reversing EMT or a chemotherapeutic agent, for example the small molecule Axl kinase inhibitor BGB324/R428/bemcentinib when administered either as a single agent or in combination with another cancer treatment.

To “respond to treatment” or “benefit from treatment” as used herein means to experience an overall clinical benefit from the treatment. This overall clinical benefit can be any of: prolonged survival, partial or complete disease remission, slowing or absence of disease progression, tumour shrinkage (for example, a reduction in tumour volume of 5, 10, 20, 30, 40% or more), reduction in tumour burden (for example, a reduction in tumour burden of 5, 10, 20, 30, 40% or more), slowing or absence of tumour enlargement, slowing or absence of increase in tumour burden, improved quality of life, progression-free survival, overall survival, or any other positive patient outcome. Suitable methods for determining tumour volume are well known to the skilled person, for example, using: computed tomography (CT), or magnetic resonance imaging (MRI) imaging technologies; X-ray imaging, for example, mammography; ultrasound imaging; nuclear imaging, for example positron emission tomography (PET), PET/CT scans, bone scans, gallium scans, or metaiodobenzylguanidine (MIBG) scans; bioluminescence imaging (BLI); fluorescence imaging (FLI); BD ToF (infrared-based 3D Time-of-Flight camera) imaging.

Biomarkers

The term “marker” or “biomarker” is used herein to refer to a gene or protein whose expression in a subject or sample derived from a subject is altered or modulated, for example, up or down regulated, in cancer. Where the biomarker is a protein, modulation or alteration of expression encompasses modulation through different post-translational modifications.

In accordance with any aspect of this facet of the present disclosure the biomarkers described herein may be defined as follows, wherein:
an Axl biomarker comprises an amino acid sequence having at least 70%, 80%, 90%, 95%, 99% or 100% sequence identity with the full-length of the amino acid sequence disclosed at UniProt accession no: P30530-1 or P30530-2 (entry version 196) or a fragment thereof;
a Beta Amyloid 1-40 (AB-40) biomarker comprises an amino acid sequence having at least 70%, 80%, 90%, 95%, 99% or 100% sequence identity with the full-length of the amino acid sequence disclosed at UniProt accession no: P05067-1, P05067-2, P05067-3, P05067-4, P05067-5, P05067-6, P05067-7, P05067-8, P05067-9, P05067-10, or P05067-11 (entry version 271) or a fragment thereof;
an Aggrecan core protein (Aggrecan) biomarker comprises an amino acid sequence having at least 70%, 80%, 90%, 95%, 99% or 100% sequence identity with the full-length of the amino acid sequence disclosed at UniProt accession no: P16112-1, P16112-2, or P16112-3 (entry version 197) or a fragment thereof;
a Ferritin (FRTN) biomarker comprises an amino acid sequence having at least 70%, 80%, 90%, 95%, 99% or 100% sequence identity with the full-length of the amino acid sequence disclosed at UniProt accession no: P02794 (entry version 213) or a fragment thereof;
a Receptor for advanced glycosylation end products (RAGE) biomarker comprises an amino acid sequence having at least 70%, 80%, 90%, 95%, 99% or 100% sequence identity with the full-length of the amino acid sequence disclosed at UniProt accession no: Q15109-1, Q15109-2, Q15109-3, Q15109-4, Q15109-5, Q15109-6, Q15109-7, Q15109-8, Q15109-9, Q15109-10 (entry version 189) or a fragment thereof;
a Neuronal Cell Adhesion Molecule (Nr-CAM) biomarker comprises an amino acid sequence having at least 70%, 80%, 90%, 95%, 99% or 100% sequence identity with the full-length of the amino acid sequence disclosed at UniProt accession no: Q92823-1, Q92823-2, Q92823-3, Q92823-4, Q92823-5, or Q92823-6 (entry version 172) or a fragment thereof;
a Sclerostin biomarker comprises an amino acid sequence having at least 70%, 80%, 90%, 95%, 99% or 100% sequence identity with the full-length of the amino acid sequence disclosed at UniProt accession no: Q9BQB4-1 or Q9BQB4-2 (entry version 145) or a fragment thereof;
a Prostate-Specific Antigen, Free (PSA-f) biomarker comprises an amino acid sequence having at least 70%, 80%, 90%, 95%, 99% or 100% sequence identity with the full-length of the amino acid sequence disclosed at UniProt accession no: P07288-1, P07288-2, P07288-3, P07288-4, or P07288-5 (entry version 197) or a fragment thereof;
an Epidermal Growth Factor Receptor (EGFR) biomarker comprises an amino acid sequence having at least 70%, 80%, 90%, 95%, 99% or 100% sequence identity with the full-length of the amino acid sequence disclosed at UniProt accession no: Q504U8 (entry version 138) or a fragment thereof;
a Neurofilament heavy polypeptide (NF-H) biomarker comprises an amino acid sequence having at least 70%, 80%, 90%, 95%, 99% or 100% sequence identity with the full-length of the amino acid sequence disclosed at UniProt accession no: P12036-1 or P12036-2 (entry version 177) or a fragment thereof;
a Dopamine beta-hydroxylase (DBH) biomarker comprises an amino acid sequence having at least 70%, 80%, 90%, 95%, 99% or 100% sequence identity with the full-length of the amino acid sequence disclosed at UniProt accession no: P09172 (entry version 194) or a fragment thereof;
a Fibulin-1C (Fib-1C) biomarker comprises an amino acid sequence having at least 70%, 80%, 90%, 95%, 99% or 100% sequence identity with the full-length of the amino acid sequence disclosed at UniProt accession no: P23142-1, P23142-2, P23142-3, or P23142-4 (entry version 217) or a fragment thereof;
a Myoglobin biomarker comprises an amino acid sequence having at least 70%, 80%, 90%, 95%, 99% or 100% sequence identity with the full-length of the amino acid sequence disclosed at UniProt accession no: P02144 (entry version 178) or a fragment thereof;
a Urokinase-type Plasminogen Activator (uPA) biomarker comprises an amino acid sequence having at least 70%, 80%, 90%, 95%, 99% or 100% sequence identity with the full-length of the amino acid sequence disclosed at UniProt accession no: P00749-1 or P00749-2 (entry version 230) or a fragment thereof;
a Visceral adipose tissue-derived serpin A12 (Vaspin) biomarker comprises an amino acid sequence having at least 70%, 80%, 90%, 95%, 99% or 100% sequence identity with the full-length of the amino acid sequence disclosed at UniProt accession no: Q8IW75 (entry version 122) or a fragment thereof;
an Interleukin-23 (IL-23) biomarker comprises an amino acid sequence having at least 70%, 80%, 90%, 95%, 99% or 100% sequence identity with the full-length of the amino acid sequence disclosed at UniProt accession no: Q9NPF7 (entry version 133) or a fragment thereof;
an Alpha-2-Macroglobulin (A2Macro) biomarker comprises an amino acid sequence having at least 70%, 80%, 90%, 95%, 99% or 100% sequence identity with the full-length of the amino acid sequence disclosed at UniProt accession no: P01023 (entry version 206) or a fragment thereof;
a Tissue Inhibitor of Metalloproteinases 2 (TIMP-2) biomarker comprises an amino acid sequence having at least 70%, 80%, 90%, 95%, 99% or 100% sequence identity with the full-length of the amino acid sequence disclosed at UniProt accession no: P16035 (entry version 187) or a fragment thereof;
a Lactate Dehydrogenase (LDH) biomarker comprises an amino acid sequence having at least 70%, 80%, 90%, 95%, 99% or 100% sequence identity with the full-length of the amino acid sequence disclosed at UniProt accession no: P00338-1, P00338-2, P00338-3, P00338-4, or P00338-5 (entry version 224) or a fragment thereof; and/or
a Lactate Dehydrogenase (LDH) biomarker comprises an amino acid sequence having at least 70%, 80%, 90%, 95%, 99% or 100% sequence identity with the full-length of the amino acid sequence disclosed at UniProt accession no: P07195-1 (entry version 218) or a fragment thereof.

In accordance with any aspect of this facet of the present disclosure the biomarkers described herein may also be defined as follows, wherein:
an Axl biomarker comprises an amino acid sequence having at least 70%, 80%, 90%, 95%, 99% or 100% sequence identity with the full-length of the amino acid sequence disclosed at UniProt accession no: P30530-1 or P30530-2 (entry version 196) or a fragment thereof;
a Thyroid-Stimulating Hormone (TSH) biomarker comprises an amino acid sequence having at least 70%, 80%, 90%, 95%, 99% or 100% sequence identity with the full-length of the amino acid sequence disclosed at UniProt accession no: P01222-1 or P01222-2 (entry version 161) or a fragment thereof;
a Tetranectin biomarker comprises an amino acid sequence having at least 70%, 80%, 90%, 95%, 99% or 100% sequence identity with the full-length of the amino acid sequence disclosed at UniProt accession no: P05452 (entry version 177) or a fragment thereof;
a Myeloid Progenitor Inhibitory Factor 1 (MPIF-1) biomarker comprises an amino acid sequence having at least 70%, 80%, 90%, 95%, 99% or 100% sequence identity with the full-length of the amino acid sequence disclosed at UniProt accession no: P55773-1 or P55773-2 (entry version 162) or a fragment thereof;
a Pancreatic Polypeptide (PPP) biomarker comprises an amino acid sequence having at least 70%, 80%, 90%, 95%, 99% or 100% sequence identity with the full-length of the amino acid sequence disclosed at UniProt accession no: P01298-1 or P01298-2 (entry version 175) or a fragment thereof;
a Visceral adipose tissue-derived serpin A12 (Vaspin) biomarker comprises an amino acid sequence having at least 70%, 80%, 90%, 95%, 99% or 100% sequence identity with the full-length of the amino acid sequence disclosed at UniProt accession no: Q8IW75 (entry version 122) or a fragment thereof;
an Interleukin-7 (IL-7) biomarker comprises an amino acid sequence having at least 70%, 80%, 90%, 95%, 99% or 100% sequence identity with the full-length of the amino acid sequence disclosed at UniProt accession no: P13232-1, P13232-2, or P13232-3 (entry version 170) or a fragment thereof;
a Pulmonary and Activation-Regulated Chemokine (PARC) biomarker comprises an amino acid sequence having at least 70%, 80%, 90%, 95%, 99% or 100% sequence identity with the full-length of the amino acid sequence disclosed at UniProt accession no: P55774 (entry version 152) or a fragment thereof;
a Cancer Antigen 15-3 (CA-15-3) biomarker comprises an amino acid sequence having at least 70%, 80%, 90%, 95%, 99% or 100% sequence identity with the full-length of the amino acid sequence disclosed at UniProt accession no: P15941-1, P15941-2, P15941-3, P15941-4, P15941-5, P15941-6, P15941-7, P15941-8, P15941-9, P15941-10, P15941-11, P15941-12, P15941-13, P15941-14, P15941-15, P15941-16, or P15941-17 (entry version 204) or a fragment thereof;
a Lactate Dehydrogenase (LDH) biomarker comprises an amino acid sequence having at least 70%, 80%, 90%, 95%, 99% or 100% sequence identity with the full-length of the amino acid sequence disclosed at UniProt accession no: P00338-1, P00338-2, P00338-3, P00338-4, or P00338-5 (entry version 224) or a fragment thereof; and/or
a Lactate Dehydrogenase (LDH) biomarker comprises an amino acid sequence having at least 70%, 80%, 90%, 95%, 99% or 100% sequence identity with the full-length of the amino acid sequence disclosed at UniProt accession no: P07195-1 (entry version 218) or a fragment thereof.

In accordance with any aspect of this facet of the present disclosure the biomarkers described herein may also be defined as follows, wherein:
an Axl biomarker comprises an amino acid sequence having at least 70%, 80%, 90%, 95%, 99% or 100% sequence identity with the full-length of the amino acid sequence disclosed at UniProt accession no: P30530-1 or P30530-2 (entry version 196) or a fragment thereof;
a Pepsinogen I (PGI) biomarker comprises an amino acid sequence having at least 70%, 80%, 90%, 95%, 99% or 100% sequence identity with the full-length of the amino acid sequence disclosed at UniProt accession no: P0DJD8 (entry version 50) or a fragment thereof;
an Apolipoprotein E (Apo E) biomarker comprises an amino acid sequence having at least 70%, 80%, 90%, 95%, 99% or 100% sequence identity with the full-length of the amino acid sequence disclosed at UniProt accession no: P02649 (entry version 230) or a fragment thereof;
an Apolipoprotein C-I (Apo C-I) biomarker comprises an amino acid sequence having at least 70%, 80%, 90%, 95%, 99% or 100% sequence identity with the full-length of the amino acid sequence disclosed at UniProt accession no: P02654 (entry version 175) or a fragment thereof;
a Macrophage-Stimulating Protein (MSP) biomarker comprises an amino acid sequence having at least 70%, 80%, 90%, 95%, 99% or 100% sequence identity with the full-length of the amino acid sequence disclosed at UniProt accession no: P26927 (entry version 179) or a fragment thereof;
a C-C motif chemokine 15 (CCL15) biomarker comprises an amino acid sequence having at least 70%, 80%, 90%, 95%, 99% or 100% sequence identity with the full-length of the amino acid sequence disclosed at UniProt accession no: Q16663 (entry version 161) or a fragment thereof;
a Progranulin biomarker comprises an amino acid sequence having at least 70%, 80%, 90%, 95%, 99% or 100% sequence identity with the full-length of the amino acid sequence disclosed at UniProt accession no: P28799-1, P28799-2, or P28799-3 (entry version 197) or a fragment thereof;
a Carcinoembryonic antigen-related cell adhesion molecule 1 (CEACAM1) biomarker comprises an amino acid sequence having at least 70%, 80%, 90%, 95%, 99% or 100% sequence identity with the full-length of the amino acid sequence disclosed at UniProt accession no: P13688-1, P13688-2, P13688-3, P13688-4, P13688-5, P13688-6, P13688-7, P13688-8, P13688-9, P13688-10, or P13688-11 (entry version 197) or a fragment thereof;
a Vitamin D-Binding Protein (VDBP) biomarker comprises an amino acid sequence having at least 70%, 80%, 90%, 95%, 99% or 100% sequence identity with the full-length of the amino acid sequence disclosed at UniProt accession no: P02774-1, P02774-2, or P02774-3 (entry version 201) or a fragment thereof;
a Carbonic anhydrase 9 (CA-9) biomarker comprises an amino acid sequence having at least 70%, 80%, 90%, 95%, 99% or 100% sequence identity with the full-length of the amino acid sequence disclosed at UniProt accession no: Q16790 (entry version 171) or a fragment thereof;
a Lactate Dehydrogenase (LDH) biomarker comprises an amino acid sequence having at least 70%, 80%, 90%, 95%, 99% or 100% sequence identity with the full-length of the amino acid sequence disclosed at UniProt accession no: P00338-1, P00338-2, P00338-3, P00338-4, or P00338-5 (entry version 224) or a fragment thereof; and/or
a Lactate Dehydrogenase (LDH) biomarker comprises an amino acid sequence having at least 70%, 80%, 90%, 95%, 99% or 100% sequence identity with the full-length of the amino acid sequence disclosed at UniProt accession no: P07195-1 (entry version 218) or a fragment thereof.

The biomarkers described herein may also be defined as a nucleic acid (either DNA or RNA) having at least 70%, 80%, 90%, 95%, 99% or 100% sequence identity with the full-length nucleic acid sequence encoding any of the above amino acid sequences.

Identity may be as defined using sequence comparisons made using FASTA and FASTP (see Pearson & Lipman, 1988. Methods in Enzymology 183: 63-98). Parameters are preferably set, using the default matrix, as follows: Gapopen (penalty for the first residue in a gap): −12 for proteins/-16 for DNA; Gapext (penalty for additional residues in a gap): −2 for proteins/-4 for DNA; KTUP word length: 2 for proteins/6 for DNA.

Pre-Treatment Predictive Biomarkers and Methods

By analysing patient data obtained from clinical trial BGBC008 (NCT03184571), BGBIL005 (NCT02922777), and BGBC004 (NCT02424617), the authors have discovered that, prior to treatment of subjects having NSCLC, certain of the biomarkers described herein are present in the serum of “responder” subjects at higher levels than in the serum of “non-responder” subjects—see Examples 4, 5, 6, and 7. Similarly, prior to treatment of the subject certain of the biomarkers described herein are present in the serum of “responder” subjects at lower levels than in the serum of “non-responder” subjects. Thus, by assessing the activity, expression, or amount of one or more of the biomarkers described herein prior to treatment with an agent capable of inhibiting or reversing EMT or a chemotherapeutic agent it is possible to determine the likelihood of that subject being susceptible or responsive to treatment with said agent.

Accordingly, in a first aspect of this facet, the disclosure provides a method of predicting a cancer-related outcome in a subject having, suspected of having, or diagnosed with non-small cell lung cancer (NSCLC), the method comprising assessing the activity, expression, or amount of one or more of the biomarkers described herein in the subject, or in a sample derived from the subject.

In some embodiments the one or more biomarker is selected from the group consisting of: Axl, Beta Amyloid 1-40 (AB-40), Aggrecan core protein (Aggrecan), Ferritin (FRTN), Receptor for advanced glycosylation end products (RAGE), Neuronal Cell Adhesion Molecule (Nr-CAM), Sclerostin, Prostate-Specific Antigen, Free (PSA-f), Epidermal Growth Factor Receptor (EGFR), Neurofilament heavy polypeptide (NF-H), Dopamine beta-hydroxylase (DBH), Fibulin-1C (Fib-1C), Myoglobin, Urokinase-type Plasminogen Activator (uPA), Visceral adipose tissue-derived serpin A12 (Vaspin), Interleukin-23 (IL-23), Alpha-2-Macroglobulin (A2Macro), Tissue Inhibitor of Metalloproteinases 2 (TIMP-2), Thyroid-Stimulating Hormone (TSH), Tetranectin, Myeloid Progenitor Inhibitory Factor 1 (MPIF-1), Pancreatic Polypeptide (PPP), Interleukin-7 (IL-7), Pulmonary and Activation-Regulated Chemokine (PARC), Cancer Antigen 15-3 (CA-15-3), Pepsinogen I (PGI), Apolipoprotein E (Apo E), Apolipoprotein C-I (Apo C-I), Macrophage-Stimulating Protein (MSP), C-C motif chemokine 15 (CCL15), Progranulin, Carcinoembryonic antigen-related cell adhesion molecule 1 (CEACAM1), Vitamin D-Binding Protein (VDBP), Carbonic anhydrase 9 (CA-9), and/or Lactate Dehydrogenase (LDH).

In some embodiments the one or more biomarker is selected from the group consisting of: Axl, Beta Amyloid 1-40 (AB-40), Aggrecan core protein (Aggrecan), Ferritin (FRTN), Receptor for advanced glycosylation end products (RAGE), Neuronal Cell Adhesion Molecule (Nr-CAM), Sclerostin, Prostate-Specific Antigen, Free (PSA-f), Epidermal Growth Factor Receptor (EGFR), Neurofilament heavy polypeptide (NF-H), Dopamine beta-hydroxylase (DBH), Fibulin-1C (Fib-1C), Myoglobin, Urokinase-type Plasminogen Activator (uPA), Visceral adipose tissue-derived serpin A12 (Vaspin), Interleukin-23 (IL-23), Alpha-2-Macroglobulin (A2Macro), Tissue Inhibitor of Metalloproteinases 2 (TIMP-2), and/or Lactate Dehydrogenase (LDH).

In some embodiments the one or more biomarker is selected from the group consisting of: Axl, Beta Amyloid 1-40 (AB-40), Aggrecan core protein (Aggrecan), Ferritin (FRTN), Receptor for advanced glycosylation end products (RAGE), Neuronal Cell Adhesion Molecule (Nr-CAM), Sclerostin, Prostate-Specific Antigen, Free (PSA-f), Epidermal Growth Factor Receptor (EGFR), Neurofilament heavy polypeptide (NF-H), Dopamine beta-hydroxylase (DBH), Fibulin-1C (Fib-1C), Myoglobin, Urokinase-type Plasminogen Activator (uPA), Visceral adipose tissue-derived serpin A12 (Vaspin), Interleukin-23 (IL-23), Alpha-2-Macroglobulin (A2Macro), and/or Tissue Inhibitor of Metalloproteinases 2 (TIMP-2).

In some embodiments the one or more biomarker is selected from the group consisting of: Axl, Thyroid-Stimulating Hormone (TSH), Tetranectin, Myeloid Progenitor Inhibitory Factor 1 (MPIF-1), Pancreatic Polypeptide (PPP), Visceral adipose tissue-derived serpin A12 (Vaspin), Interleukin-7 (IL-7), Pulmonary and Activation-Regulated Chemokine (PARC), Cancer Antigen 15-3 (CA-15-3), and/or Lactate Dehydrogenase (LDH).

In some embodiments the one or more biomarker is selected from the group consisting of: Axl, Thyroid-Stimulating Hormone (TSH), Tetranectin, Myeloid Progenitor Inhibitory Factor 1 (MPIF-1), Pancreatic Polypeptide (PPP), Visceral adipose tissue-derived serpin A12 (Vaspin), Interleukin-7 (IL-7), Pulmonary and Activation-Regulated Chemokine (PARC), and/or Cancer Antigen 15-3 (CA-15-3).

In some embodiments the one or more biomarker is selected from the group consisting of: Axl, Pepsinogen I (PGI), Apolipoprotein E (Apo E), Apolipoprotein C-I (Apo C-I), Macrophage-Stimulating Protein (MSP), C-C motif chemokine 15 (CCL15), Progranulin, Carcinoembryonic antigen-related cell adhesion molecule 1 (CEACAM1), Vitamin D-Binding Protein (VDBP), Carbonic anhydrase 9 (CA-9), and/or Lactate Dehydrogenase (LDH).

In some embodiments the one or more biomarker is selected from the group consisting of: Axl, Pepsinogen I (PGI), Apolipoprotein E (Apo E), Apolipoprotein C-I (Apo C-I), Macrophage-Stimulating Protein (MSP), C-C motif chemokine 15 (CCL15), Progranulin, Carcinoembryonic antigen-related cell adhesion molecule 1 (CEACAM1), Vitamin D-Binding Protein (VDBP), and/or Carbonic anhydrase 9 (CA-9).

A “cancer-related outcome” as described herein is a clinical prediction or prognosis associated with cancer. In some embodiments the cancer-related outcome is susceptibility to treatment with an agent capable of inhibiting or reversing EMT or a chemotherapeutic agent.

In some embodiments the method comprises assessing the activity, expression, or amount of one or more of the biomarkers described herein in the subject, or in a sample derived from the subject, to obtain a sample profile of the one or more biomarker; and, making a prediction based on the sample profile of the one or more biomarker.

A “sample profile” as used herein is a profile of numerical values or numerical ranges corresponding to the activity, expression, or amount of respective biomarkers determined in the subject or sample derived from the subject.

In some embodiments of the method the prediction is made by comparing the sample profile to a control profile. A “control profile” as used herein is a profile of numerical values or numerical ranges corresponding to the activity, expression, or amount of the same biomarkers as for the sample profile, determined in a control subject or population of control subjects. Suitable control profiles are described in more detail below.

Prior to treatment of a subject, certain of the biomarkers described herein have been found to exhibit up-regulated expression in “responder” subjects—that is, certain of the biomarkers described herein have been found to be present in the serum of “responder” subjects at higher levels than in the serum of “non-responder” subjects. Similarly, prior to treatment of a subject, certain of the biomarkers described herein have been found to exhibit down-regulated expression in “responder” subjects—that is, certain of the biomarkers described herein have been found to be present in the serum of “responder” subjects at lower levels than in the serum of “non-responder” subjects.

This difference in expression level between “responder” and “non-responder” subjects can also be expressed in the opposite way. That is to say, prior to treatment of a subject, certain of the biomarkers described herein have been found to exhibit down-regulated expression in “non-responder” subjects—that is, certain of the biomarkers described herein have been found to be present in the serum of “non-responder” subjects at lower levels than in the serum of “responder” subjects. Similarly, prior to treatment of a subject, certain of the biomarkers described herein have been found to exhibit up-regulated expression in “non-responder” subjects—that is, certain of the biomarkers described herein have been found to be present in the serum of “non-responder” subjects at higher levels than in the serum of “responder” subjects.

The discovery of these differential patterns of expression mean that, by assessing the activity, expression, or amount of one or more of the biomarkers described herein prior to treatment of a subject with an agent, it is possible to determine the likelihood of that subject being susceptible or responsive to treatment with said agent. Accordingly, in particularly preferred embodiments the sample profile is obtained before the subject is contacted with or administered the agent capable of inhibiting or reversing EMT or a chemotherapeutic agent.

A subject who is “susceptible to treatment” with an agent capable of inhibiting or reversing EMT or a chemotherapeutic agent is one who is likely to, or who does, respond to treatment (or benefit from treatment) with an agent capable of inhibiting or reversing EMT or a chemotherapeutic agent. That is, a subject who is “susceptible to treatment” with an agent is a “responder” subject for that agent, as defined above. A prediction that is “indicative of susceptibility” is one which suggests or points towards the likelihood that a subject will respond to or benefit from treatment.

In some embodiments the predictive methods of the disclosure comprise assessing the activity, expression, or amount of one or more of the biomarkers described herein in the subject, or in a sample derived from the subject, to obtain a sample profile of the one or more biomarker; and, making a prediction based on the sample profile of the one or more biomarker. In some embodiments of the method the prediction is made by comparing the sample profile to a control profile, wherein the control profile is a profile of numerical values or numerical ranges corresponding to the activity, expression, or amount of the same biomarkers as for the sample profile, determined in a control subject or population of control subjects.

In some embodiments the control profile may be obtained from a population of control subjects having NSCLC. In some embodiments the control profile may be obtained from a population of control subjects not having NSCLC. In other embodiments the control profile may be obtained from a control subject or population of control subjects having NSCLC and previously found to lack susceptibility to treatment with an agent capable of inhibiting or reversing EMT or a chemotherapeutic agent (that is, non-responder subjects).

In some embodiments the control profile may a predetermined profile of biomarker expression, activity, or amount values, for example a profile of “mean” values, “threshold” values, or “standard ranges” of values. This predetermined profile of biomarker expression, activity, or amount values can be obtained, for example, from a control subject or population of control subjects having NSCLC, a control subject or population of control subjects not having NSCLC, or a control subject or population of control subjects having NSCLC and previously found to lack susceptibility to treatment with an agent capable of inhibiting or reversing EMT or a chemotherapeutic agent (that is, non-responder subjects).

In some other embodiments the control profile may be obtained from a control subject or population of control subjects having NSCLC and previously found to be susceptible to treatment with an agent capable of inhibiting or reversing EMT or a chemotherapeutic agent (that is, responder subjects). In some embodiments the control profile may a predetermined profile of biomarker expression, activity, or amount values, for example a profile of “threshold”, values or “standard ranges” of values. This predetermined profile of biomarker expression, activity, or amount values can be obtained, for example, from a control subject or population of control subjects having NSCLC and previously found to be susceptible to treatment with an agent capable of inhibiting or reversing EMT or a chemotherapeutic agent (that is, responder subjects).

In some other embodiments the control profile may be obtained from a control sample having a known amount (for example a “threshold” amount) of the same biomarkers as will be determined in the sample profile. This threshold amount can be obtained, for example, from non-responder or responder subjects as outlined above.

The skilled person is readily able to determine suitable “mean”, “threshold”, or “standard range” values for a particular biomarker.

For example, a “mean” value may be determined by: assessing the activity, expression, or amount of a particular biomarker in a population of subjects having (or not having) NSCLC; and, averaging the biomarker activity, expression, or amount determined to determine a “mean” value.

For example, a “mean” value may be determined by: assessing the activity, expression, or amount of a particular biomarker in a population of subjects; determining which subjects in the population are susceptible to treatment with an agent capable of inhibiting or reversing EMT or a chemotherapeutic agent; and, averaging the biomarker activity, expression, or amount determined in subjects found to be susceptible to treatment with an agent capable of inhibiting or reversing EMT or a chemotherapeutic agent (that is, responder subjects) to determine a “mean” value.

For example, a “threshold” value may be determined by: assessing the activity, expression, or amount of a particular biomarker in a population of subjects; determining which subjects in the population are susceptible to treatment with an agent capable of inhibiting or reversing EMT or a chemotherapeutic agent; and, selecting as appropriate the highest or lowest biomarker activity, expression, or amount determined in a subject found to be susceptible to treatment with an agent capable of inhibiting or reversing EMT or a chemotherapeutic agent (that is, a responder subject) as the “threshold value”.

For example, a “standard range” of values may be determined by: assessing the activity, expression, or amount of a particular biomarker in a population of subjects having (or not having) NSCLC; and, determining a “standard range” of values based on this assessment.

For example, a “standard range” of values may be determined by: assessing the activity, expression, or amount of a particular biomarker in a population of subjects; determining which subjects in the population are susceptible to treatment with an agent capable of inhibiting or reversing EMT or a chemotherapeutic agent; and, determining a “standard range” of values for subjects found to be susceptible to treatment with an agent capable of inhibiting or reversing EMT or a chemotherapeutic agent (that is, a responder subject) based on this assessment.

Where the control profile is a predetermined profile of biomarker expression, activity, or amount values, this may be provided as a “look-up table” or data record. Predetermined control profiles of this type may be a profile of numerical values or numerical ranges corresponding to a measure of central tendency (such as an average, median or mean) amount of the respective biomarkers determined in a plurality of control subjects (for example non-responder or responder subjects as outlined above). The skilled person is readily able to determine both a measure of central tendency and a measure of variability within a population using standard protein quantification techniques and suitable statistical tools.

Accordingly, in embodiments in which the control profile is: obtained from a population of control subjects having NSCLC; obtained from a population of control subjects not having NSCLC; obtained from a control subject or population of control subjects having NSCLC and previously found to lack susceptibility to treatment with an agent capable of inhibiting or reversing EMT or a chemotherapeutic agent (that is non-responder subjects); a predetermined profile of biomarker expression, activity, or amount values (for example a profile of “average, median, or mean” values or “standard ranges” of values) obtained from non-responder subjects; obtained from a control sample having a known “average, median, or mean” value of biomarkers indicative of non-responder subjects; a predetermined profile of biomarker expression, activity, or amount “threshold” values obtained from responder subjects; or, obtained from a control sample having a known “threshold” value of biomarkers indicative of responder subjects; then, a higher activity, expression, or amount of one or more of the biomarkers described herein in a sample profile as compared with the control profile may be indicative of susceptibility to treatment with an agent capable of inhibiting or reversing EMT or a chemotherapeutic agent. Similarly, a lower activity, expression, or amount of one or more of the biomarkers described herein in the sample profile as compared with a control profile may be indicative of susceptibility to treatment with an agent capable of inhibiting or reversing EMT or a chemotherapeutic agent.

In some such embodiments, the method may comprise assessing the activity, expression, or amount of one or more of biomarker selected from: Nr-CAM, Sclerostin, PSA-f, EGFR, NF-H, DBH, Fib-1C, Myoglobin, uPA, Vaspin, IL-23, A2Macro, TIMP-2, and/or LDH; wherein a higher activity, expression, or amount of one or more of said biomarkers in the sample profile as compared with the control profile is indicative of susceptibility to treatment with an agent capable of inhibiting or reversing EMT or a chemotherapeutic agent.

In some such embodiments, the method may comprise assessing the activity, expression, or amount of one or more of biomarker selected from: Axl, AB-40, Aggrecan, FRTN, and/or RAGE; wherein a lower activity, expression, or amount of one or more of said biomarkers in the sample profile as compared with the control profile is indicative of susceptibility to treatment with an agent capable of inhibiting or reversing EMT or a chemotherapeutic agent.

In some such embodiments: a lower activity, expression, or amount of Axl; and/or a lower activity, expression, or amount of AB-40; and/or a lower activity, expression, or amount of Aggrecan; and/or a lower activity, expression, or amount of FRTN; and/or a lower activity, expression, or amount of RAGE; and/or a higher activity, expression, or amount of Nr-CAM; and/or a higher activity, expression, or amount of Sclerostin; and/or a higher activity, expression, or amount of PSA-f; and/or a higher activity, expression, or amount of EGFR; and/or a higher activity, expression, or amount of NF-H; and/or a higher activity, expression, or amount of DBH; and/or a higher activity, expression, or amount of Fib-1C; and/or a higher activity, expression, or amount of Myoglobin; and/or a higher activity, expression, or amount of uPA; and/or a higher activity, expression, or amount of Vaspin; and/or a higher activity, expression, or amount of IL-23; and/or a higher activity, expression, or amount of A2Macro; and/or a higher activity, expression, or amount of TIMP-2; and/or a higher activity, expression, or amount of LDH; in the sample profile as compared with the control profile is indicative of susceptibility to treatment with an agent capable of inhibiting or reversing EMT or a chemotherapeutic agent.

In other such embodiments, the method may comprise assessing the activity, expression, or amount of one or more of biomarker selected from: MPIF-1, PPP, Vaspin, IL-7, PARC, CA-15-3, and/or LDH; wherein a higher activity, expression, or amount of one or more of said biomarkers in the sample profile as compared with the control profile is indicative of susceptibility to treatment with an agent capable of inhibiting or reversing EMT or a chemotherapeutic agent.

In other such embodiments, the method may comprise assessing the activity, expression, or amount of one or more of biomarker selected from: Axl, TSH, and/or Tetranectin; wherein a lower activity, expression, or amount of one or more of said biomarkers in the sample profile as compared with the control profile is indicative of susceptibility to treatment with an agent capable of inhibiting or reversing EMT or a chemotherapeutic agent.

In other such embodiments: a lower activity, expression, or amount of Axl; and/or a lower activity, expression, or amount of TSH; and/or a lower activity, expression, or amount of Tetranectin; and/or a higher activity, expression, or amount of MPIF-1; and/or a higher activity, expression, or amount of PPP; and/or a higher activity, expression, or amount of Vaspin; and/or a higher activity, expression, or amount of IL-7; and/or a higher activity, expression, or amount of PARC; and/or a higher activity, expression, or amount of CA-15-3; and/or a higher activity, expression, or amount of LDH; in the sample profile as compared with the control profile is indicative of susceptibility to treatment with an agent capable of inhibiting or reversing EMT or a chemotherapeutic agent.

In other such embodiments, the method may comprise assessing the activity, expression, or amount of one or more of biomarker selected from: VDBP, CA-9, and/or LDH; wherein a higher activity, expression, or amount of one or more of said biomarkers in the sample profile as compared with the control profile is indicative of susceptibility to treatment with an agent capable of inhibiting or reversing EMT or a chemotherapeutic agent.

In other such embodiments, the method may comprise assessing the activity, expression, or amount of one or more of biomarker selected from: Axl, PGI, Apo E, Apo C-I, MSP, CCL15, Progranulin, and/or CEACAM1; wherein a lower activity, expression, or amount of one or more of said biomarkers in the sample profile as compared with the control profile is indicative of susceptibility to treatment with an agent capable of inhibiting or reversing EMT or a chemotherapeutic agent.

In other such embodiments: a lower activity, expression, or amount of Axl; and/or a lower activity, expression, or amount of PGI; and/or a lower activity, expression, or amount of Apo E; and/or a lower activity, expression, or amount of Apo C-I; and/or a lower activity, expression, or amount of MSP; and/or a lower activity, expression, or amount of CCL15; and/or a lower activity, expression, or amount of Progranulin; and/or a lower activity, expression, or amount of CEACAM1; and/or a higher activity, expression, or amount of VDBP; and/or a higher activity, expression, or amount of CA-9; and/or a higher activity, expression, or amount of LDH; in the sample profile as compared with the control profile is indicative of susceptibility to treatment with an agent capable of inhibiting or reversing EMT or a chemotherapeutic agent.

In embodiments in which the control profile is: obtained from a control subject or population of control subjects having NSCLC and previously found to be susceptible to treatment with an agent capable of inhibiting or reversing EMT or a chemotherapeutic agent (that is, responder subjects); then, if the expression, activity, or amount values in the sample profile are above a “threshold” value typical of a responder subject (that is, a subject having NSCLC and previously found to be susceptible to treatment with an agent capable of inhibiting or reversing EMT or a chemotherapeutic agent) this is indicative of susceptibility to treatment with an agent capable of inhibiting or reversing EMT or a chemotherapeutic agent. Similarly, if the expression, activity, or amount values in the sample profile are below the “threshold” value typical of a responder subject (that is, a subject having NSCLC and previously found to be susceptible to treatment with an agent capable of inhibiting or reversing EMT or a chemotherapeutic agent) this is indicative of a lack of susceptibility to treatment with an agent capable of inhibiting or reversing EMT or a chemotherapeutic agent.

In embodiments in which the control profile is: a predetermined profile of “standard ranges” of biomarker expression, activity, or amount values obtained from responder subjects; then, if the expression, activity, or amount values in the sample profile are within the “standard range” of values typical of a responder subject (that is, a subject having NSCLC and previously found to be susceptible to treatment with an agent capable of inhibiting or reversing EMT or a chemotherapeutic agent) this is indicative of susceptibility to treatment with an agent capable of inhibiting or reversing EMT or a chemotherapeutic agent. Similarly, if the expression, activity, or amount values in the sample profile are outside the “standard range” of values typical of a responder subject (that is, a subject having NSCLC and previously found to be susceptible to treatment with an agent capable of inhibiting or reversing EMT or a chemotherapeutic agent) this is indicative of a lack of susceptibility to treatment with an agent capable of inhibiting or reversing EMT or a chemotherapeutic agent.

In embodiments in which the control profile is: a predetermined profile of “standard ranges” of biomarker expression, activity, or amount values obtained from non-responder subjects; then, if the expression, activity, or amount values in the sample profile are within the “standard range” of values typical of a non-responder subject (that is, a subject having NSCLC and previously found to lack susceptibility to treatment with an agent capable of inhibiting or reversing EMT or a chemotherapeutic agent) this is indicative of a lack of susceptibility to treatment with an agent capable of inhibiting or reversing EMT or a chemotherapeutic agent. Similarly, if the expression, activity, or amount values in the sample profile are outside the “standard range” of values typical of a non-responder subject (that is, a subject having NSCLC and previously found to lack susceptibility to treatment with an agent capable of inhibiting or reversing EMT or a chemotherapeutic agent) this may be indicative of susceptibility to treatment with an agent capable of inhibiting or reversing EMT or a chemotherapeutic agent.

In embodiments in which the control profile is: obtained from a population of control subjects not having NSCLC; then, if the expression, activity, or amount values in the sample profile are outside a “standard range” of values typical of subjects not having NSCLC, this is indicative of susceptibility to treatment with an agent capable of inhibiting or reversing EMT or a chemotherapeutic agent. Similarly, if the expression, activity, or amount values in the sample profile are within a “standard range” of values typical of subjects not having NSCLC, this is indicative of a lack of susceptibility to treatment with an agent capable of inhibiting or reversing EMT or a chemotherapeutic agent. In such cases, an expression, activity or amount value may be outside the “standard range” of values when it is either above the upper limit of normal or below the lower limit of normal for a given biomarker. The upper limit of normal and lower limit of normal are the limits of the normal distribution of a given biomarker in a population of control subjects not having NSCLC.

In embodiments in which the one or more biomarker comprises LDH, an expression or amount of LDH higher than 287 units per litre (U/L) may be indicative of susceptibility to treatment with an agent capable of inhibiting or reversing EMT or a chemotherapeutic agent.

The skilled person is readily able to determine whether the sample profile is indicative of susceptibility or lack of susceptibility to treatment with an agent capable of inhibiting or reversing EMT or a chemotherapeutic agent using suitable statistical tools and comparison with the appropriate control.

In some embodiments the sample profile may be compared with two or more control profiles as described above.

As used herein, the term “one or more” means 1, 2, 3, 4, 5, 6, 7, 8, 9, 10, 11, 12, 13, 14, 15, 16, 17, 18, 19, or 20 of the biomarkers described herein. In some embodiments the one or more biomarker is Axl. In some embodiments the one or more biomarker is AB-40. In some embodiments the one or more biomarker is Aggrecan. In some embodiments the one or more biomarker is FRTN. In some embodiments the one or more biomarker is RAGE. In some embodiments the one or more biomarker is Nr-CAM. In some embodiments the one or more biomarker is Sclerostin. In some embodiments the one or more biomarker is PSA-f. In some embodiments the one or more biomarker is EGFR. In some embodiments the one or more biomarker is NF-H. In some embodiments the one or more biomarker is DBH. In some embodiments the one or more biomarker is Fib-1C. In some embodiments the one or more biomarker is Myoglobin. In some embodiments the one or more biomarker is uPA. In some embodiments the one or more biomarker is Vaspin. In some embodiments the one or more biomarker is IL-23. In some embodiments the one or more biomarker is A2Macro. In some embodiments the one or more biomarker is TIMP-2. In some embodiments the one or more biomarker is LDH. In some preferred embodiments the one or more biomarker is Axl. In some preferred embodiments the one or more biomarker is LDH. In other preferred embodiments the one or more biomarker is Axl and LDH.

In some embodiments the one or more biomarker is Axl. In some embodiments the one or more biomarker is TSH. In some embodiments the one or more biomarker is Tetranectin. In some embodiments the one or more biomarker is MPIF-1. In some embodiments the one or more biomarker is PPP. In some embodiments the one or more biomarker is Vaspin. In some embodiments the one or more biomarker is IL-7. In some embodiments the one or more biomarker is PARC. In some embodiments the one or more biomarker is CA-15-3. In some embodiments the one or more biomarker is LDH. In some preferred embodiments the one or more biomarker is Axl. In some preferred embodiments the one or more biomarker is LDH. In other preferred embodiments the one or more biomarker is Axl and LDH.

In some embodiments the one or more biomarker is Axl. In some embodiments the one or more biomarker is PGI. In some embodiments the one or more biomarker is Apo E. In some embodiments the one or more biomarker is Apo C-I. In some embodiments the one or more biomarker is MSP. In some embodiments the one or more biomarker is CCL15. In some embodiments the one or more biomarker is Progranulin. In some embodiments the one or more biomarker is CEACAM1. In some embodiments the one or more biomarker is VDBP. In some embodiments the one or more biomarker is CA-9. In some embodiments the one or more biomarker is LDH. In some preferred embodiments the one or more biomarker is Axl. In some preferred embodiments the one or more biomarker is CA-9. In some preferred embodiments the one or more biomarker is LDH. In other preferred embodiments the one or more biomarker is Axl and CA-9. In other preferred embodiments the one or more biomarker is Axl and LDH. In other preferred embodiments the one or more biomarker is CA-9 and LDH. In other preferred embodiments the one or more biomarker is Axl, CA-9, and LDH.

In some embodiments the method may comprise assessing the activity, expression, or amount of at least 2, 3, 4, 5, 6, 7, 8, 9, 10, 11, 12, 13, 14, 15, 20 of said biomarkers in the subject, or in a sample derived from the subject. In some preferred embodiments the method may comprise assessing the activity, expression, or amount of Axl and at least 1, 2, 3, 4, 5, 6, 7, 8, 9, 10, 11, 12, 13, 14, 15, 20 further biomarkers described herein in the subject, or in a sample derived from the subject. In some preferred embodiments the method may comprise assessing the activity, expression, or amount of LDH and at least 1, 2, 3, 4, 5, 6, 7, 8, 9, 10, 11, 12, 13, 14, 15, 20 further biomarkers described herein in the subject, or in a sample derived from the subject. In some preferred embodiments the method may comprise assessing the activity, expression, or amount of CA-9, and at least 1, 2, 3, 4, 5, 6, 7, 8, 9, 10, 11, 12, 13, 14, 15, 20 further biomarkers described herein in the subject, or in a sample derived from the subject. In some preferred embodiments the method may comprise assessing the activity, expression, or amount of Axl, LDH, and at least 1, 2, 3, 4, 5, 6, 7, 8, 9, 10, 11, 12, 13, 14, 15, 20 further biomarkers described herein in the subject, or in a sample derived from the subject. In other preferred embodiments the method may comprise assessing the activity, expression, or amount of Axl, CA-9, and at least 1, 2, 3, 4, 5, 6, 7, 8, 9, 10, 11, 12, 13, 14, 15, 20 further biomarkers described herein in the subject, or in a sample derived from the subject. In some preferred embodiments the method may comprise assessing the activity, expression, or amount of CA-9, LDH, and at least 1, 2, 3, 4, 5, 6, 7, 8, 9, 10, 11, 12, 13, 14, 15, 20 further biomarkers described herein in the subject, or in a sample derived from the subject. In some preferred embodiments the method may comprise assessing the activity, expression, or amount of Axl, CA-9, LDH, and at least 1, 2, 3, 4, 5, 6, 7, 8, 9, 10, 11, 12, 13, 14, 15, 20 further biomarkers described herein in the subject, or in a sample derived from the subject.

Subject

A “subject” as referred to herein is any species of animal classified as a mammal, and includes but is not restricted to, domestic and farm animals, primates and humans. In some preferred embodiments the subject is a human of any sex or race. In some embodiments, the human is an adult human.

In some embodiments the predictive methods described herein are performed before the subject receives a treatment or course of treatment for NSCLC. In other embodiments the predictive method is performed during or after a treatment or course of treatment for NSCLC. In some embodiments the treatment or course of treatment for NSCLC is an agent capable of inhibiting or reversing EMT. In other embodiments the treatment or course of treatment for NSCLC is a chemotherapeutic treatment that is not an agent capable of inhibiting or reversing EMT.

In some embodiments of the predictive methods described herein the subject has previously been treated with an agent capable of inhibiting or reversing EMT or a chemotherapeutic agent. In some embodiments the subject has previously been treated with an agent selected from: a PHGDH inhibitor, a Slfn11 inhibitor, an Axl inhibitor, or an Akt3 inhibitor.

In some embodiments of the predictive methods described herein the subject has previously been treated with an agent that is different to the agent capable of inhibiting or reversing EMT or chemotherapeutic agent for which the subject's response is being predicted. That is, the subject has previously been treated with an agent that is not the agent which the predictive method is determining the subject's susceptibility to. Accordingly, in some embodiments, the subject has not previously been treated with an agent capable of inhibiting or reversing EMT or a chemotherapeutic agent. In some preferred embodiments, the subject has not previously been treated with an Axl inhibitor. In other preferred embodiments, the subject has not previously been treated with an Akt3 inhibitor.

Post-Treatment Changes in Biomarkers

In some embodiments of this aspect of the disclosure, the predictive method further comprises a subsequent step of assessing the activity, expression, or amount of one or more biomarker in the subject, or in a sample derived from the subject, to obtain a second sample profile of one or more of the biomarkers described herein after the subject has received a treatment or course of treatment an agent capable of inhibiting or reversing EMT or a chemotherapeutic agent.

That is, in some embodiments, the predictive method comprises:
(a) determining if a subject is susceptible to treatment with an agent capable of inhibiting or reversing EMT or a chemotherapeutic agent by assessing the activity, expression, or amount of one or more biomarker in the subject, as outlined above;
(b) administering to the subject a therapeutically effective amount of an agent capable of inhibiting or reversing EMT or a chemotherapeutic agent; and
(c) subsequently assessing the activity, expression, or amount of one or more biomarker in the subject, or in a sample derived from the subject.

In some embodiments, the predictive method comprises:
(a) determining if a subject is susceptible to treatment with an agent capable of inhibiting or reversing EMT or a chemotherapeutic agent by assessing the activity, expression, or amount of one or more biomarker in the subject, or in a sample derived from the subject, wherein the one or more biomarker is selected from the group consisting of:
(i) Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, TIMP-2, and/or LDH;
(ii) Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, and/or TIMP-2;
(iii) Axl, TSH, MPIF-1, PPP, Vaspin, IL-7, PARC, CA-15-3, and/or LDH;
(iv) Axl, TSH, MPIF-1, PPP, Vaspin, IL-7, PARC, and/or CA-15-3;
(v) Axl, PGI, Apo E, Apo C-I, MSP, CCL15, Progranulin, CEACAM1, VDBP, CA-9, and/or LDH; or
(vi) Axl, PGI, Apo E, Apo C-I, MSP, CCL15, Progranulin, CEACAM1, VDBP, and/or CA-9;
(b) administering to the subject a therapeutically effective amount of an agent capable of inhibiting or reversing EMT or a chemotherapeutic agent; and
(c) subsequently assessing the activity, expression, or amount of one or more biomarker in the subject, or in a sample derived from the subject.

In some preferred embodiments the methods comprise subsequently assessing the activity, expression, or amount of Axl in the subject, or in a sample derived from the subject. In some embodiments an increase in the activity, expression, or amount of Axl in the second sample profile as compared with a control profile after contacting the subject with a cancer therapeutic agent is indicative of higher susceptibility to treatment with an agent capable of inhibiting or reversing EMT or a chemotherapeutic agent. In some preferred embodiments the control profile is obtained from the same subject prior to treatment with an agent capable of inhibiting or reversing EMT or a chemotherapeutic agent.

Post-Treatment Predictive Biomarkers and Methods

By analysing patient data obtained from clinical trial BGBC008 (NCT03184571), BGBIL005 (NCT02922777), and BGBC004 (NCT02424617), the authors have discovered that, following treatment of subjects having NSCLC with an agent capable of inhibiting or reversing EMT or a chemotherapeutic agent, the levels of certain of the biomarkers described herein increases in the serum of “responder” subjects but not in the serum of “non-responder” subjects. Similarly, following treatment with an agent capable of inhibiting or reversing EMT or a chemotherapeutic agent, the levels of certain of the biomarkers described herein decreases in the serum of “responder” subjects but not in the serum of “non-responder” subjects. Thus, by assessing the activity, expression, or amount of one or more of the biomarkers described herein prior to and following treatment with an agent capable of inhibiting or reversing EMT or a chemotherapeutic agent it is possible to determine or confirm that the subject is susceptible or responsive to treatment with said agent.

Accordingly, in a second aspect of this facet the disclosure provides a method of predicting a cancer-related outcome in a subject having, suspected of having, or diagnosed with non-small cell lung cancer (NSCLC), the method comprising assessing the activity, expression, or amount of one or more of the biomarkers described herein in a subject, or in a sample derived from the subject after the subject has been contacted with an agent capable of inhibiting or reversing EMT or a chemotherapeutic agent. In some embodiments the cancer-related outcome is susceptibility to treatment with an agent capable of inhibiting or reversing EMT or a chemotherapeutic agent.

In some embodiments the method comprises assessing the activity, expression, or amount of one or more of the biomarkers described herein in the subject, or in a sample derived from the subject, to obtain a sample profile of the one or more biomarker; and, making a prediction based on the sample profile of the one or more biomarker. In some embodiments of the method the prediction is made by comparing the sample profile to a control profile. In preferred embodiments the sample profile is obtained after contacting the subject with an agent capable of inhibiting or reversing EMT or a chemotherapeutic agent.

A “control profile” as used herein is a profile of numerical values or numerical ranges corresponding to the activity, expression, or amount of the same biomarkers as for the sample profile, determined in a control subject or population of control subjects.

In preferred embodiments of this aspect of the disclosure the control profile is obtained from the same subject prior to treatment with an agent capable of inhibiting or reversing EMT or a chemotherapeutic agent. In some embodiments the control profile is obtained from a population of control subjects having NSCLC. In other embodiments, the control profile is a predetermined profile of biomarker expression, activity, or amount values, for example a profile of “threshold” values or “standard ranges” of values.

Accordingly, in embodiments in which the control profile is obtained from the same subject prior to treatment with an agent capable of inhibiting or reversing EMT or a chemotherapeutic agent, an increase in the activity, expression, or amount of one or more biomarker in the sample profile as compared with a control profile after contacting the subject with an agent capable of inhibiting or reversing EMT or a cancer therapeutic agent may be indicative of susceptibility to treatment with an agent capable of inhibiting or reversing EMT or a chemotherapeutic agent. In such embodiments, a decrease in the activity, expression, or amount of one or more biomarker in the sample profile as compared with a control profile after contacting the subject with a cancer therapeutic agent may be indicative of higher susceptibility to treatment with an agent capable of inhibiting or reversing EMT or a chemotherapeutic agent.

In some such embodiments, an increase in the activity, expression, or amount of one or more biomarker in the sample profile as compared with a control profile after contacting the subject with a cancer therapeutic agent may be indicative of lower susceptibility to treatment with an agent capable of inhibiting or reversing EMT or a chemotherapeutic agent. In some such embodiments, a decrease in the activity, expression, or amount of one or more biomarker in the sample profile as compared with a control profile after contacting the subject with a cancer therapeutic agent may be indicative of lower susceptibility to treatment with an agent capable of inhibiting or reversing EMT or a chemotherapeutic agent.

In some preferred embodiments the one or more biomarker includes at least Axl. In some embodiments the one or more biomarker is Axl. In some embodiments an increase in the activity, expression, or amount of Axl in the sample profile as compared with a control profile after contacting the subject with a cancer therapeutic agent is indicative of higher susceptibility to treatment with an agent capable of inhibiting or reversing EMT or a chemotherapeutic agent.

Subject

A “subject” as referred to herein is any species of animal classified as a mammal, and includes but is not restricted to, domestic and farm animals, primates and humans. In some preferred embodiments the subject is a human of any sex or race. In some embodiments, the human is an adult human.

In some embodiments of this aspect of the disclosure, the subject was selected for treatment with the agent capable of inhibiting or reversing EMT or a chemotherapeutic agent, using a predictive method according to the first aspect of this facet of the disclosure outlined above.

Agents Capable of Inhibiting or Reversing EMT

The present disclosure provides methods for predicting a cancer-related outcome in a subject having, suspected of having, or diagnosed with non-small cell lung cancer (NSCLC). A “cancer-related outcome” is a clinical prediction or prognosis associated with cancer.

The agents capable of inhibiting or reversing EMT for use in this NSCLC facet of the disclosure are defined as described above in the AML facet (see identically titled section above on page 14, line 19 to page 17, line 2).

In some preferred embodiments the further cancer treatment is a PD-1 inhibitor or PD-L1 inhibitor. In some embodiments the further cancer treatment is a monoclonal antibody such as pembrolizumab, alemtuzumab, bevacizumab, cetuximab, ibritumomab, tiuxetan, or rituximab. In some preferred embodiments the further cancer treatment is a microtubule polymer stabilizer such as the taxanes paclitaxel and docetaxel. In some preferred embodiments the further cancer treatment is a tyrosine kinase inhibitors such as erlotinib hydrochloride, gefitinib, imatinib mesylate, and semaxanib. In some particularly preferred embodiments the further cancer treatment is pembrolizumab. In other particularly preferred embodiments the further cancer treatment is docetaxel. In yet other particularly preferred embodiments the further cancer treatment is erlotinib.

Assessing Biomarker Activity, Expression, Amount

In some embodiments of the predictive methods described herein, assessing the activity, expression, or amount of one or more biomarker in a subject, or in a sample derived from a subject, comprises: contacting a sample, or an extract from a sample, with at least 1, 2, 3, 4, 5, 6, 7, 8, 9, 10 or more reagents for detecting the activity, expression, or amount of one or more biomarkers, each of which is selective for a particular biomarker; and, detecting and/or quantifying said reagents for detecting.

In some embodiments comprising assessing the activity of LDH, assessing the activity of LDH comprises determining the enzymatic activity of LDH in the subject or sample derived from the subject. Preferably the enzymatic activity is assessed by determining the level or amount of a substrate or product of the enzymatic reaction catalysed by LDH in the subject or sample derived from the subject. In particularly preferred embodiments the enzymatic activity is assessed by determining the level or amount of reduced nicotinamide adenine dinucleotide (NADH) in the subject or sample derived from the subject. In some embodiments this may be determined using a colorimetric assay, for example, the colorimetric assay available from Sigma-Aldrich with catalogue number MAK066. Other suitable assays for assessing activity of LDH are well known to the skilled person. In particularly preferred embodiments the activity of LDH is assessed by determining the enzymatic activity of LDH in blood or serum.

In some preferred embodiments, assessing the expression or amount of the one or more biomarker comprises determining the level of the one or more biomarker protein or mRNA in the subject or sample derived from the subject. Preferably the expression or amount of the one or more biomarker is assessed by determining the level of protein expression. In particularly preferred embodiments the expression or amount of the one or more biomarker is assessed by determining the level of protein expression in serum.

The term “expression” refers to the transcription of a gene's DNA template to produce the corresponding mRNA and translation of this mRNA to produce the corresponding gene product (i.e., a peptide, polypeptide, or protein) as well as the “expression” of a protein in one or more forms that may have been modified post translation.

In some embodiments determining the level of the one or more biomarker protein or mRNA comprises: contacting a sample, or an extract from a sample, with at least 1, 2, 3, 4, 5, 6, 7, 8, 9, 10 or more specific binding members, each of which selectively binds to a respective biomarker protein or mRNA; and, detecting and/or quantifying formation of complex formed by said specific binding member and said biomarker protein or mRNA. In some embodiments the specific binding member may comprise an antibody molecule or a binding fragment thereof.

A range of suitable techniques for determining protein and mRNA expression levels are well known in the art, for example microarray analysis, Western blotting, and PCR techniques such as QPCR. In some embodiments, determining the expression or amount of the one or more biomarker protein or mRNA may comprise use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.

In some embodiments, the expression or amount of the one or more biomarker is determined as an absolute amount of the respective biomarker protein or mRNA in the sample. In such cases, the respective biomarker protein or mRNA may be quantified in the sample and the amount expressed as a measure of the sample, for example the volume, mass, or total protein content of the sample.

In some embodiments, the expression or amount of the one or more biomarker is determined as a relative amount of the respective biomarker protein or mRNA in the sample, wherein the relative amount is determined relative to a reference protein or mRNA in the sample. Determining the relative amount of biomarker protein or mRNA in this way allows for normalisation to, e.g., account for differences in total protein concentration and to remove bias from sample to sample.

Preferably the reference protein or mRNA is one whose expression or amount does not vary significantly between subjects having cancer and subjects not having cancer, in particular NSCLC. That is, the reference protein or mRNA is preferably one whose expression or amount is not altered by NSCLC. Accordingly, the reference protein or mRNA may comprise one or more protein or mRNA which is not one of the biomarkers described herein. When expressing the expression or amount of the one or more biomarker as a relative amount, the absolute amount of the respective biomarker protein or mRNA in the sample may be divided by the absolute amount of the reference protein or mRNA in the sample.

Assessing Biomarker Expression at the RNA Level

Gene expression can be detected at the RNA level. RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland). Typical assay formats utilising ribonucleic acid hybridisation include nuclear run-on assays, RT-PCR, RNase protection assays (Melton et al., Nuc. Acids Res. 12:7035), Northern blotting and In Situ hybridization. Gene expression can also be detected by microarray analysis as described below.

Assessing Biomarker Expression at the Protein Level

Preferably biomarker level is assessed by measuring protein expression. Altered gene or protein expression may also be detected by measuring the polypeptides encoded by the gene. This may be achieved by using molecules which bind to the polypeptides encoded by the biomarker gene. Suitable molecules/agents which bind either directly or indirectly to the polypeptides in order to detect the presence of the protein include naturally occurring molecules such as peptides and proteins, for example antibodies, or they may be synthetic molecules.

Antibodies may be derived from commercial sources or through techniques which are familiar to those skilled in the art. In one embodiment, and where altered expression manifests itself through the expression of alteration of post translationally-modified forms of a protein biomarker, antibodies specific for those different forms may be used. For the purposes of this disclosure, the term “antibody”, unless specified to the contrary, includes whole antibodies, or fragments of whole antibodies which retain their binding activity for a target antigen. Such fragments include Fv, F(ab′) and F(ab′)2 fragments, as well as single chain antibodies (scFv). Furthermore, the antibodies and fragments thereof may be humanised antibodies, for example as described in EP239400A. For example: monoclonal and polyclonal antibodies, recombinant antibodies, proteolytic and recombinant fragments of antibodies (Fab, Fv, scFv, diabodies), single-domain antibodies (VHH, sdAb, nanobodies, IgNAR, VNAR), and proteins unrelated to antibodies, which have been engineered to have antibody-like specific binding. Antibodies may be bound to a solid support and/or packaged into kits in a suitable container along with suitable reagents, controls, instructions and the like.

Arrays

Array technology and the various techniques and applications associated with it are well known in the art. Array technology overcomes the disadvantages with traditional methods in molecular biology, which generally work on a “one gene in one experiment” basis, resulting in low throughput and the inability to appreciate the “whole picture” of gene function. In the context of the products and methods of the present disclosure, array technology can be used, for example, in the analysis of the expression of biomarker proteins or mRNA.

In general, any library or group of samples may be arranged in an orderly manner into an array, by spatially separating the members of the library or group. Examples of suitable libraries for arraying include nucleic acid libraries (including DNA, cDNA, oligonucleotide, etc. libraries), peptide, polypeptide and protein libraries, as well as libraries comprising any molecules, such as ligand libraries, among others. Accordingly, where reference is made to a “library” in this document, unless the context dictates otherwise, such reference should be taken to include reference to a library in the form of an array.

Proteins, polypeptides, etc., may also be immobilised in arrays. For example, antibodies have been used in microarray analysis of the proteome using protein chips (Borrebaeck C A, 2000, Immunol Today 21(8):379-82). Polypeptide arrays are reviewed in, for example, MacBeath and Schreiber, 2000, Science, 289(5485):1760-1763.

Sample

Suitable samples include, but are not limited to, tissue samples such as tissue biopsy, blood, urine, buccal scrapes etc., as well as serum, plasma, or tissue culture supernatant samples. In preferred embodiments the expression or amount of the one or more biomarker is assessed by determining the level of protein expression in the sample. In some preferred embodiments the sample is a blood, serum, or plasma sample. In some particularly preferred embodiments the sample is a serum sample.

When assessing the expression or amount of the one or more biomarker proteins or mRNA in serum or plasma samples derived from a subject, samples are removed and subjected to analytical techniques such as flow cytometry, mass cytometry (CyTOF), ELISA, PET, and SELDI-TOF MS. In some embodiments, the method may comprise extracting RNA from said sample and detecting gene expression by QPCR. In some other embodiments, gene expression may be detected by detecting protein products by, for example, Western Blot.

In some embodiments, the level of expression of the one or more biomarker is assessed by determining the copy number of the gene encoding the one or more biomarker. Copy number (i.e. gene duplication events) may be determined using standard techniques known in the art, e.g. using a DNA chip as described in Jiang et al. (Jiang Q, Ho Y Y, Hao L, Nichols Berrios C, Chakravarti A. Copy number variants in candidate genes are genetic modifiers of Hirschsprung disease. PLoS One. 2011; 6(6)).

Is some preferred embodiments of the predictive methods described herein, the method is performed in vitro or ex vivo.

Methods of Selecting a Subject for Treatment

A third aspect of this facet of the present disclosure relates to methods of selecting subjects having, suspected of having, or diagnosed with non-small cell lung cancer (NSCLC) for treatment with an agent capable of inhibiting or reversing EMT or a chemotherapeutic agent. In some embodiments the method comprises: identifying subjects susceptible to treatment with an agent capable of inhibiting or reversing EMT or a chemotherapeutic agent using a predictive method defined in accordance with the above described first and second aspects of this facet of the disclosure; and, selecting thus identified subjects for treatment.

In some embodiments this aspect relates to methods of selecting subjects having, suspected of having, or diagnosed with non-small cell lung cancer (NSCLC) for continued treatment with an agent capable of inhibiting or reversing EMT or a chemotherapeutic agent. In some embodiments the method comprises: identifying subjects susceptible to treatment with an agent capable of inhibiting or reversing EMT or a chemotherapeutic agent using a predictive method defined in accordance with the above described second aspect of this facet of the disclosure; and, selecting thus identified subjects for treatment.

In some embodiments of this aspect of the disclosure, the treatment comprises administering to the subject a therapeutically effective amount of an agent capable of inhibiting or reversing EMT or a chemotherapeutic agent as described herein. Accordingly, in some embodiments the treatment comprises an Axl inhibitor, Akt3 inhibitor, Slfn11 inhibitor, or PHGDH inhibitor. In some preferred embodiments the treatment comprises an Axl inhibitor, for example, BGB324/R428/bemcentinib, cabozantinib, TP-0903, foretinib, merestinib, bosutinib, gilteritinib, crizotinib, amuvatinib, sunitinib, sitravatinib, LDC1267, or UNC2025. Other Axl inhibitors include the anti-Axl antibodies described in WO2015/193428, WO2015/193430, WO2016/097370, and WO2016/166296. In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In some other preferred embodiments, the treatment comprises an Akt3 inhibitor. In some embodiments the Akt3 inhibitor is an Akt3 inhibitor disclosed in WO2016/102672.

In some embodiments of this aspect of the disclosure, the treatment is administered as a single agent. In other embodiments, the treatment is administered in combination with a further cancer treatment. Suitable further cancer treatments are outlined in detail above.

In some preferred embodiments the further cancer treatment is a PD-1 inhibitor or PD-L1 inhibitor. In some embodiments the further cancer treatment is a monoclonal antibody such as pembrolizumab, alemtuzumab, bevacizumab, cetuximab, ibritumomab, tiuxetan, or rituximab. In some preferred embodiments the further cancer treatment is a microtubule polymer stabilizer such as the taxanes paclitaxel and docetaxel. In some preferred embodiments the further cancer treatment is a tyrosine kinase inhibitor such as erlotinib hydrochloride, gefitinib, imatinib mesylate, and semaxanib. In some particularly preferred embodiments the further cancer treatment is pembrolizumab. In other particularly preferred embodiments the further cancer treatment is docetaxel. In yet other particularly preferred embodiments the further cancer treatment is erlotinib.

Kits

A fourth aspect of this facet of the present disclosure relates to diagnostic kits and test devices. In some embodiments the disclosure provides a diagnostic kit comprising 1, 2, 3, 4, 5, 6, 7, 8, 9, 10 or more reagents for detecting the activity, expression, or amount of one or more of the biomarkers described herein, each of which is selective for a particular biomarker. In some embodiments each of the reagents for detecting is selective for a biomarker selected from the group consisting of: Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, TIMP-2, TSH, MPIF-1, PPP, IL-7, PARC, CA-15-3, PGI, Apo E, Apo C-I, MSP, CCL15, Progranulin, CEACAM1, VDBP, CA-9, and/or LDH. In some embodiments each of the reagents for detecting is selective for a biomarker selected from the group consisting of: Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, TIMP-2, TSH, MPIF-1, PPP, IL-7, PARC, CA-15-3, PGI, Apo E, Apo C-I, MSP, CCL15, Progranulin, CEACAM1, VDBP, and/or CA-9.

In other embodiments the disclosure provides a test device comprising 1, 2, 3, 4, 5, 6, 7, 8, 9, 10 or more reagents for detecting the activity, expression, or amount of one or more of the biomarkers described herein, each of which is selective for a particular biomarker. In some embodiments each of the reagents for detecting is selective for a biomarker selected from the group consisting of: Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, TIMP-2, TSH, MPIF-1, PPP, IL-7, PARC, CA-15-3, PGI, Apo E, Apo C-I, MSP, CCL15, Progranulin, CEACAM1, VDBP, CA-9, and/or LDH. In some embodiments each of the reagents for detecting is selective for a biomarker selected from the group consisting of: Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, TIMP-2, TSH, MPIF-1, PPP, IL-7, PARC, CA-15-3, PGI, Apo E, Apo C-I, MSP, CCL15, Progranulin, CEACAM1, VDBP, and/or CA-9.

In some embodiments the diagnostic kit or test device comprises 1, 2, 3, 4, 5, 6, 7, 8, 9, 10 or more specific binding members, each of which selectively binds to one of the biomarkers described herein; and, one or more reagents for detecting said one or more specific binding members, or one or more reagents for detecting and/or quantifying formation of a complex formed by said specific binding member and said biomarker. In some embodiments each specific binding member selectively binds to a biomarker selected from the group consisting of: Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, TIMP-2, TSH, MPIF-1, PPP, IL-7, PARC, CA-15-3, PGI, Apo E, Apo C-I, MSP, CCL15, Progranulin, CEACAM1, VDBP, CA-9, and/or LDH. In some embodiments each specific binding member selectively binds to a biomarker selected from the group consisting of: Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, TIMP-2, TSH, MPIF-1, PPP, IL-7, PARC, CA-15-3, PGI, Apo E, Apo C-I, MSP, CCL15, Progranulin, CEACAM1, VDBP, and/or CA-9.

In some embodiments each reagent for detecting or specific binding member is selective for a biomarker selected from the group:
(i) Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, TIMP-2, and/or LDH;
(ii) Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, and/or TIMP-2;
(iii) Axl, TSH, MPIF-1, PPP, Vaspin, IL-7, PARC, CA-15-3, and/or LDH;
(iv) Axl, TSH, MPIF-1, PPP, Vaspin, IL-7, PARC, and/or CA-15-3;
(v) Axl, PGI, Apo E, Apo C-I, MSP, CCL15, Progranulin, CEACAM1, VDBP, CA-9, and/or LDH; or
(vi) Axl, PGI, Apo E, Apo C-I, MSP, CCL15, Progranulin, CEACAM1, VDBP, and/or CA-9.

In some embodiments the diagnostic kit or test device comprises a specific binding member which selectively binds to Axl, and at least 1, 2, 3, 4, 5, 6, 7, 8, 9, 10, or more specific binding members, each of which selectively binds to a biomarker selected from the group consisting of:
(i) Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, TIMP-2, and/or LDH;
(ii) Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, and/or TIMP-2;
(iii) Axl, TSH, MPIF-1, PPP, Vaspin, IL-7, PARC, CA-15-3, and/or LDH;
(iv) Axl, TSH, MPIF-1, PPP, Vaspin, IL-7, PARC, and/or CA-15-3;
(v) Axl, PGI, Apo E, Apo C-I, MSP, CCL15, Progranulin, CEACAM1, VDBP, CA-9, and/or LDH; or
(vi) Axl, PGI, Apo E, Apo C-I, MSP, CCL15, Progranulin, CEACAM1, VDBP, and/or CA-9.

In some embodiments the diagnostic kit or test device comprises a specific binding member which selectively binds to LDH, and at least 1, 2, 3, 4, 5, 6, 7, 8, 9, 10, or more specific binding members, each of which selectively binds to a biomarker selected from the group consisting of:
(i) Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, and/or TIMP-2;
(ii) Axl, TSH, MPIF-1, PPP, Vaspin, IL-7, PARC, and/or CA-15-3; or
(iii) Axl, PGI, Apo E, Apo C-I, MSP, CCL15, Progranulin, CEACAM1, VDBP, and/or CA-9.

In some embodiments the diagnostic kit or test device comprises a specific binding member which selectively binds to Axl, a specific binding member which selectively binds to LDH, and at least 1, 2, 3, 4, 5, 6, 7, 8, 9, 10, or more specific binding members, each of which selectively binds to a biomarker selected from the group consisting of:
(i) AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, and/or TIMP-2;
(ii) TSH, MPIF-1, PPP, Vaspin, IL-7, PARC, and/or CA-15-3; or
(iii) PGI, Apo E, Apo C-I, MSP, CCL15, Progranulin, CEACAM1, VDBP, and/or CA-9.

In some embodiments the diagnostic kit or test device comprises a specific binding member which selectively binds to CA-9, and at least 1, 2, 3, 4, 5, 6, 7, 8, 9, 10, or more specific binding members, each of which selectively binds to a biomarker selected from the group consisting of: Axl, PGI, Apo E, Apo C-I, MSP, CCL15, Progranulin, CEACAM1, VDBP, and/or LDH. In some embodiments the diagnostic kit or test device comprises a specific binding member which selectively binds to CA-9, and at least 1, 2, 3, 4, 5, 6, 7, 8, 9, 10, or more specific binding members, each of which selectively binds to a biomarker selected from the group consisting of: Axl, PGI, Apo E, Apo C-I, MSP, CCL15, Progranulin, CEACAM1, and/or VDBP.

In some embodiments the diagnostic kit or test device comprises a specific binding member which selectively binds to Axl, a specific binding member which selectively binds to CA-9, and at least 1, 2, 3, 4, 5, 6, 7, 8, 9, 10, or more specific binding members, each of which selectively binds to a biomarker selected from the group consisting of: PGI, Apo E, Apo C-I, MSP, CCL15, Progranulin, CEACAM1, VDBP, and/or LDH. In some embodiments the diagnostic kit or test device comprises a specific binding member which selectively binds to Axl, a specific binding member which selectively binds to CA-9, and at least 1, 2, 3, 4, 5, 6, 7, 8, 9, 10, or more specific binding members, each of which selectively binds to a biomarker selected from the group consisting of: PGI, Apo E, Apo C-I, MSP, CCL15, Progranulin, CEACAM1, and/or VDBP.

In some embodiments the diagnostic kit or test device comprises a specific binding member which selectively binds to CA-9, a specific binding member which selectively binds to LDH, and at least 1, 2, 3, 4, 5, 6, 7, 8, 9, 10, or more specific binding members, each of which selectively binds to a biomarker selected from the group consisting of: Axl, PGI, Apo E, Apo C-I, MSP, CCL15, Progranulin, CEACAM1, and/or VDBP.

In some embodiments the diagnostic kit or test device comprises a specific binding member which selectively binds to Axl, a specific binding member which selectively binds to CA-9, a specific binding member which selectively binds to LDH, and at least 1, 2, 3, 4, 5, 6, 7, 8, 9, 10, or more specific binding members, each of which selectively binds to a biomarker selected from the group consisting of: PGI, Apo E, Apo C-I, MSP, CCL15, Progranulin, CEACAM1, and/or VDBP.

In some embodiments of this aspect of the disclosure the diagnostic kit or test device comprises a plurality of said specific binding members provided in the form of an array on a substrate or conjugated to a plurality of particles, such as beads or microspheres. The particles may be encoded with a detectable label. In some embodiments the specific binding member comprises an antibody molecule or a binding fragment thereof.

In some embodiments of the diagnostic kit or test device detecting formation of a complex formed by a specific binding member and a biomarker is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.

In some embodiments of the diagnostic kit or test device, the kit or test device comprises no more than 15, 20, 25, 30, 40, or 50 reagents for detecting. In some embodiments of the diagnostic kit or test device, the kit or test device comprises no more than 15, 20, 25, 30, 40, or 50 specific binding members.

The present disclosure also provides use of such diagnostic kits and test devices in methods of predicting a cancer-related outcome in a subject. In preferred embodiments the cancer-related outcome is susceptibility to treatment with an agent capable of inhibiting or reversing EMT or a chemotherapeutic agent as described herein.

In some embodiments the agent capable of inhibiting or reversing EMT or chemotherapeutic agent is an Axl inhibitor, Akt3 inhibitor, Slfn11 inhibitor, or PHGDH inhibitor. In some preferred embodiments the agent capable of inhibiting or reversing EMT or chemotherapeutic agent is an Axl inhibitor, for example BGB324/R428/bemcentinib, cabozantinib, TP-0903, foretinib, merestinib, bosutinib, gilteritinib, crizotinib, amuvatinib, sunitinib, sitravatinib, LDC1267, or UNC2025. Other Axl inhibitors include the anti-Axl antibodies described in WO2015/193428, WO2015/193430, WO2016/097370, and WO2016/166296. In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In some embodiments of the diagnostic kit or test device for use in a predictive method, the subject has, is suspected of having, or has been diagnosed with NSCLC.

In some embodiments the method may be as defined in accordance with the above described first and second aspects of this facet of the disclosure.

A fifth aspect of this facet of the present disclosure relates to the use of a reagent for detecting the activity, expression, or amount of a biomarker selected from the group consisting of: Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, TIMP-2, TSH, MPIF-1, PPP, IL-7, PARC, CA-15-3, PGI, Apo E, Apo C-I, MSP, CCL15, Progranulin, CEACAM1, VDBP, CA-9, and/or LDH, in a method of predicting a cancer-related outcome in a subject.

A sixth aspect of this facet of the disclosure relates to the use of 1, 2, 3, 4, 5, 6, 7, 8, 9, 10 or more reagents for detecting the activity, expression, or amount a biomarker, each of which is selective for a particular biomarker selected from the group consisting of: Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, TIMP-2, TSH, MPIF-1, PPP, IL-7, PARC, CA-15-3, PGI, Apo E, Apo C-I, MSP, CCL15, Progranulin, CEACAM1, VDBP, CA-9, and/or LDH, in a method of manufacture of a diagnostic kit or test device for use in a method of predicting a cancer-related outcome in a subject. In some embodiments of these uses the reagent for detecting comprises a specific binding member selective for a particular biomarker. The specific binding member may comprise an antibody molecule or a binding fragment thereof.

In some embodiments the reagents for detecting or specific binding members may be selected from the group consisting of:
(i) Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, TIMP-2, and/or LDH;
(ii) Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, and/or TIMP-2;
(iii) Axl, TSH, MPIF-1, PPP, Vaspin, IL-7, PARC, CA-15-3, and/or LDH;
(iv) Axl, TSH, MPIF-1, PPP, Vaspin, IL-7, PARC, and/or CA-15-3;
(v) Axl, PGI, Apo E, Apo C-I, MSP, CCL15, Progranulin, CEACAM1, VDBP, CA-9, and/or LDH; or
(vi) Axl, PGI, Apo E, Apo C-I, MSP, CCL15, Progranulin, CEACAM1, VDBP, and/or CA-9.

In some embodiments the method may be as defined in accordance with the above described first and second aspects of this facet of the disclosure.

Uses

A seventh aspect of this facet of the present disclosure relates to the use of one or more of: Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, TIMP-2, TSH, MPIF-1, PPP, IL-7, PARC, CA-15-3, PGI, Apo E, Apo C-I, MSP, CCL15, Progranulin, CEACAM1, VDBP, CA-9, and/or LDH as a biomarker for determining if a subject is susceptible to treatment with an agent capable of inhibiting or reversing EMT or a chemotherapeutic agent.

In some embodiments the use relates to use of Axl, and at least 1, 2, 3, 4, 5, 6, 7, 8, 9, 10, or more of: AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, TIMP-2, TSH, MPIF-1, PPP, IL-7, PARC, CA-15-3, PGI, Apo E, Apo C-I, MSP, CCL15, Progranulin, CEACAM1, VDBP, CA-9, and/or LDH, as a biomarker for determining if a subject is susceptible to treatment with an agent capable of inhibiting or reversing EMT or a chemotherapeutic agent. In some embodiments the use relates to use of LDH, and at least 1, 2, 3, 4, 5, 6, 7, 8, 9, 10, or more of: Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, TIMP-2, TSH, MPIF-1, PPP, IL-7, PARC, CA-15-3, PGI, Apo E, Apo C-I, MSP, CCL15, Progranulin, CEACAM1, VDBP, and/or CA-9, as a biomarker for determining if a subject is susceptible to treatment with an agent capable of inhibiting or reversing EMT or a chemotherapeutic agent.

In some embodiments the use relates to use of Axl, LDH, and at least 1, 2, 3, 4, 5, 6, 7, 8, 9, 10, or more of: AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, TIMP-2, TSH, MPIF-1, PPP, IL-7, PARC, CA-15-3, PGI, Apo E, Apo C-I, MSP, CCL15, Progranulin, CEACAM1, VDBP, and/or CA-9, as a biomarker for determining if a subject is susceptible to treatment with an agent capable of inhibiting or reversing EMT or a chemotherapeutic agent.

In some embodiments the one or more biomarker is selected from the group consisting of:
(i) Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, TIMP-2, and/or LDH;
(ii) Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, and/or TIMP-2;
(iii) Axl, TSH, MPIF-1, PPP, Vaspin, IL-7, PARC, CA-15-3, and/or LDH;
(iv) Axl, TSH, MPIF-1, PPP, Vaspin, IL-7, PARC, and/or CA-15-3;
(v) Axl, PGI, Apo E, Apo C-I, MSP, CCL15, Progranulin, CEACAM1, VDBP, CA-9, and/or LDH; or
(vi) Axl, PGI, Apo E, Apo C-I, MSP, CCL15, Progranulin, CEACAM1, VDBP, and/or CA-9.

In some preferred embodiments the one or more biomarker is Axl. In some preferred embodiments the one or more biomarker is CA-9. In some preferred embodiments the one or more biomarker is LDH. In some preferred embodiments the one or more biomarker is Axl and CA-9. In some preferred embodiments the one or more biomarker is Axl and LDH. In some preferred embodiments the one or more biomarker is CA-9 and LDH. In some preferred embodiments the one or more biomarker is Axl, CA-9, and LDH.

In other preferred embodiments the one or more biomarker is Axl, and at least 1, 2, 3, 4, 5, 6, 7, 8, 9, 10, 11, 12, 13, 14, 15, 20 further biomarkers described herein. In some other preferred embodiments the one or more biomarker is CA-9, and at least 1, 2, 3, 4, 5, 6, 7, 8, 9, 10, 11, 12, 13, 14, 15, 20 further biomarkers described herein. In some other preferred embodiments the one or more biomarker is LDH, and at least 1, 2, 3, 4, 5, 6, 7, 8, 9, 10, 11, 12, 13, 14, 15, 20 further biomarkers described herein. In some other preferred embodiments the one or more biomarker is Axl, CA-9, and at least 1, 2, 3, 4, 5, 6, 7, 8, 9, 10, 11, 12, 13, 14, 15, 20 further biomarkers described herein. In some other preferred embodiments the one or more biomarker is Axl, LDH, and at least 1, 2, 3, 4, 5, 6, 7, 8, 9, 10, 11, 12, 13, 14, 15, 20 further biomarkers described herein. In some other preferred embodiments the one or more biomarker is CA-9, LDH, and at least 1, 2, 3, 4, 5, 6, 7, 8, 9, 10, 11, 12, 13, 14, 15, 20 further biomarkers described herein. In some other preferred embodiments the one or more biomarker is Axl, CA-9, LDH, and at least 1, 2, 3, 4, 5, 6, 7, 8, 9, 10, 11, 12, 13, 14, 15, 20 further biomarkers described herein.

In some embodiments the use is as a biomarker in a method as defined in accordance with the above described first and second aspects of this facet of the disclosure.

Methods of Treating a Subject

A eighth aspect of this facet of the present disclosure relates to methods of treating a subject having, suspected of having, or diagnosed with non-small cell lung cancer (NSCLC). In some embodiments the method comprises: identifying a subject for treatment using a predictive method defined in accordance with the above described first and second aspects of this facet of the disclosure; and, administering to the subject a therapeutically effective amount of an agent capable of inhibiting or reversing EMT or a chemotherapeutic agent.

Accordingly, in some embodiments, the method comprises: (a) obtaining a sample from the subject; (b) determining if the subject is susceptible to treatment with an agent capable of inhibiting or reversing EMT or a chemotherapeutic agent using a predictive method defined in accordance with the above described first and second aspects of this facet of the disclosure; and, (c) administering to the subject a therapeutically effective amount of an agent capable of inhibiting or reversing EMT or a chemotherapeutic agent.

In some embodiments the method comprises administering a therapeutically effective amount of an agent capable of inhibiting or reversing EMT or chemotherapeutic agent to a subject identified as susceptible to said treatment using a predictive method defined in accordance with the above described first and second aspects of this facet of the disclosure.

In other embodiments the method comprises: (a) administering to a subject a therapeutically effective amount of an agent capable of inhibiting or reversing EMT or a chemotherapeutic agent; and, (b) determining if the subject is susceptible to said treatment using a predictive method defined in accordance with the above described second aspect of this facet of the disclosure. In some embodiments the method further comprises: (c) administering to the subject one or more further therapeutically effective amounts of an agent capable of inhibiting or reversing EMT or a chemotherapeutic agent provided that the subject is identified as susceptible to said treatment.

A ninth aspect of this facet of the present disclosure relates to an agent capable of inhibiting or reversing EMT or chemotherapeutic agent for use in a method of treating a subject having, suspected of having, or diagnosed with non-small cell lung cancer (NSCLC). In some embodiments the method of treatment is a method as defined above.

A tenth aspect of this facet of the present disclosure relates to use of an agent capable of inhibiting or reversing EMT or chemotherapeutic agent for use in a method of manufacture of a medicament for use in a method of treating a subject having, suspected of having, or diagnosed with non-small cell lung cancer (NSCLC). In some embodiments the method of treatment is a method as defined above.

In some embodiments of these methods, agents for use, or uses, the agent capable of inhibiting or reversing EMT or chemotherapeutic agent comprises an Axl inhibitor, Akt3 inhibitor, Slfn11 inhibitor, or PHGDH inhibitor. In some preferred embodiments the agent capable of inhibiting or reversing EMT or chemotherapeutic agent is an Axl inhibitor, for example BGB324/R428/bemcentinib, cabozantinib, TP-0903, foretinib, merestinib, bosutinib, gilteritinib, crizotinib, amuvatinib, sunitinib, sitravatinib, LDC1267, or UNC2025. Other Axl inhibitors include the anti-Axl antibodies described in WO2015/193428, WO2015/193430, WO2016/097370, and WO2016/166296. In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In some other preferred embodiments, the treatment comprises an Akt3 inhibitor. In some embodiments the Akt3 inhibitor is an Akt3 inhibitor disclosed in WO2016/102672.

In some embodiments of these methods, agents for use, or uses, the treatment is administered as a single agent. In other embodiments, the treatment is administered in combination with a further cancer treatment. Suitable further cancer treatments are outlined in detail above.

In some preferred embodiments the further cancer treatment is a PD-1 inhibitor or PD-L1 inhibitor. In some embodiments the further cancer treatment is a monoclonal antibody such as pembrolizumab, alemtuzumab, bevacizumab, cetuximab, ibritumomab, tiuxetan, or rituximab. In some preferred embodiments the further cancer treatment is a microtubule polymer stabilizer such as the taxanes paclitaxel and docetaxel. In some preferred embodiments the further cancer treatment is a tyrosine kinase inhibitor such as erlotinib hydrochloride, gefitinib, imatinib mesylate, and semaxanib. In some particularly preferred embodiments the further cancer treatment is pembrolizumab. In other particularly preferred embodiments the further cancer treatment is docetaxel. In yet other particularly preferred embodiments the further cancer treatment is erlotinib.

Statements of Disclosure

The following numbered statements relate to aspects of this facet of the present disclosure, and form part of the description:
101. A method of predicting a cancer-related outcome in a subject, the method comprising assessing the activity, expression, or amount of one or more biomarker in the subject, or in a sample derived from the subject;
   wherein the one or more biomarker is selected from the group consisting of: Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, TIMP-2, TSH, MPIF-1, PPP, IL-7, PARC, CA-15-3, PGI, Apo E, Apo C-I, MSP, CCL15, Progranulin, CEACAM1, VDBP, CA-9, and/or LDH; and
   wherein the subject has, is suspected of having, or has been diagnosed with non-small cell lung cancer (NSCLC).
102. A method according to statement 101, wherein the method comprises:
   assessing the activity, expression, or amount of one or more biomarker in the subject, or in a sample derived from the subject, to obtain a sample profile of the one or more biomarker; and
   making a prediction based on the sample profile of the one or more biomarker.
103. A method according to statement 102, wherein the prediction is made by comparing the sample profile to a control profile.
104. A method according to statement 103, wherein the sample profile is obtained before the subject is contacted with or administered an agent capable of inhibiting or reversing EMT or a chemotherapeutic agent.
105. A method according to statement 104, wherein the control profile is:
   (i) obtained from a population of control subjects having NSCLC; or
   (ii) obtained from a control subject or population of control subjects having NSCLC and previously found to lack susceptibility to treatment with an agent capable of inhibiting or reversing EMT or a chemotherapeutic agent (that is, non-responder subjects);
   (iii) a predetermined profile of biomarker expression, activity, or amount values, for example a profile of “average, median, or mean” values or “standard ranges” of values, obtained from a control subject or population of control subjects having NSCLC and previously found to lack susceptibility to treatment with an agent capable of inhibiting or reversing EMT or a chemotherapeutic agent (that is, non-responder subjects);
   (iv) obtained from a control sample having a known “average, median, or mean” value of biomarkers indicative of a control subject or population of control subjects having NSCLC and previously found to lack susceptibility to treatment with an agent capable of inhibiting or reversing EMT or a chemotherapeutic agent (that is, non-responder subjects);
   (v) a predetermined profile of biomarker expression, activity, or amount “threshold” values obtained from a control subject or population of control subjects having NSCLC and previously found to be susceptible to treatment with an agent capable of inhibiting or reversing EMT or a chemotherapeutic agent (that is, responder subjects); or
   (vi) obtained from a control sample having a known “threshold” value of biomarkers indicative of a control subject or population of control subjects having NSCLC and previously found to be susceptible to treatment with an agent capable of inhibiting or reversing EMT or a chemotherapeutic agent (that is, responder subjects).
106. A method according to statement 105, wherein a higher activity, expression, or amount of one or more biomarker in the sample profile as compared with the control profile is indicative of susceptibility to treatment with an agent capable of inhibiting or reversing EMT or a chemotherapeutic agent.
107. A method according to statement 105, wherein a lower activity, expression, or amount of one or more biomarker in the sample profile as compared with the control profile is indicative of susceptibility to treatment with an agent capable of inhibiting or reversing EMT or a chemotherapeutic agent.
108. A method according to any one of statements 105-107, wherein the one or more biomarker includes at least one of: Nr-CAM, Sclerostin, PSA-f, EGFR, NF-H, DBH, Fib-1C, Myoglobin, uPA, Vaspin, IL-23, A2Macro, TIMP-2, MPIF-1, PPP, IL-7, PARC, CA-15-3, VDBP, CA-9, and/or LDH
   and wherein a higher activity, expression, or amount of one or more of said biomarkers in the sample profile as compared with the control profile is indicative of susceptibility to treatment with an agent capable of inhibiting or reversing EMT or a chemotherapeutic agent.
109. A method according to any one of statements 105-108, wherein the one or more biomarker includes at least one of: Axl, AB-40, Aggrecan, FRTN, RAGE, TSH, Tetranectin, PGI, Apo E, Apo C-I, MSP, CCL15, Progranulin, CEACAM1, and/or LDH,
   and wherein a lower activity, expression, or amount of one or more of said biomarkers in the sample profile as compared with the control profile is indicative of susceptibility to treatment with an agent capable of inhibiting or reversing EMT or a chemotherapeutic agent.
110. A method according to any one of statements 101-109, wherein the one or more biomarker comprises a group of biomarkers selected from:
   (i) Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, TIMP-2, and/or LDH;
   (ii) Axl, TSH, MPIF-1, PPP, Vaspin, IL-7, PARC, CA-15-3, and/or LDH; or
   (iii) Axl, PGI, Apo E, Apo C-I, MSP, CCL15, Progranulin, CEACAM1, VDBP, CA-9, and/or LDH.
111. A method according to any one of statements 101-110, wherein the one or more biomarker comprises Axl, and/or CA-9, and/or LDH, optionally wherein
   (a) the one or more biomarker comprises Axl and at least 1, 2, 3, 4, 5, 6, 7, 8, 9, 10, or more further biomarkers selected from:
      (i) AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, TIMP-2, and/or LDH;
      (ii) TSH, MPIF-1, PPP, Vaspin, IL-7, PARC, CA-15-3, and/or LDH; or
      (iii) PGI, Apo E, Apo C-I, MSP, CCL15, Progranulin, CEACAM1, VDBP, CA-9, and/or LDH;
   (b) the one or more biomarker comprises CA-9 and at least 1, 2, 3, 4, 5, 6, 7, 8, 9, 10, or more further biomarkers selected from: Axl, PGI, Apo E, Apo C-I, MSP, CCL15, Progranulin, CEACAM1, and/or VDBP; or
   (c) the one or more biomarker comprises Axl, CA-9, and at least 1, 2, 3, 4, 5, 6, 7, 8, 9, 10, or more further biomarkers selected from: PGI, Apo E, Apo C-I, MSP, CCL15, Progranulin, CEACAM1, and/or VDBP.
112. A method according to any one of statements 101-111, wherein:
   said Axl biomarker comprises an amino acid sequence having at least 70%, 80%, 90%, 95%, 99% or 100% sequence identity with the full-length of the amino acid sequence disclosed at UniProt accession no: P30530-1 or P30530-2 (entry version 196) or a fragment thereof;
   said Beta Amyloid 1-40 (AB-40) biomarker comprises an amino acid sequence having at least 70%, 80%, 90%, 95%, 99% or 100% sequence identity with the full-length of the amino acid sequence disclosed at UniProt accession no: P05067-1, P05067-2, P05067-3, P05067-4, P05067-5, P05067-6, P05067-7, P05067-8, P05067-9, P05067-10, or P05067-11 (entry version 271) or a fragment thereof;
   said Aggrecan core protein (Aggrecan) biomarker comprises an amino acid sequence having at least 70%, 80%, 90%, 95%, 99% or 100% sequence identity with the full-length of the amino acid sequence disclosed at UniProt accession no: P16112-1, P16112-2, or P16112-3 (entry version 197) or a fragment thereof;
   said Ferritin (FRTN) biomarker comprises an amino acid sequence having at least 70%, 80%, 90%, 95%, 99% or 100% sequence identity with the full-length of the amino acid sequence disclosed at UniProt accession no: P02794 (entry version 213) or a fragment thereof;
   said Receptor for advanced glycosylation end products (RAGE) biomarker comprises an amino acid sequence having at least 70%, 80%, 90%, 95%, 99% or 100% sequence identity with the full-length of the amino acid sequence disclosed at UniProt accession no: Q15109-1, Q15109-2, Q15109-3, Q15109-4, Q15109-5, Q15109-6, Q15109-7, Q15109-8, Q15109-9, Q15109-10 (entry version 189) or a fragment thereof;
   said Neuronal Cell Adhesion Molecule (Nr-CAM) biomarker comprises an amino acid sequence having at least 70%, 80%, 90%, 95%, 99% or 100% sequence identity with the full-length of the amino acid sequence disclosed at UniProt accession no: Q92823-1, Q92823-2, Q92823-3, Q92823-4, Q92823-5, or Q92823-6 (entry version 172) or a fragment thereof;
   said Sclerostin biomarker comprises an amino acid sequence having at least 70%, 80%, 90%, 95%, 99% or 100% sequence identity with the full-length of the amino acid sequence disclosed at UniProt accession no: Q9BQB4-1 or Q9BQB4-2 (entry version 145) or a fragment thereof;
   said Prostate-Specific Antigen, Free (PSA-f) biomarker comprises an amino acid sequence having at least 70%, 80%, 90%, 95%, 99% or 100% sequence identity with the full-length of the amino acid sequence disclosed at UniProt accession no: P07288-1, P07288-2, P07288-3, P07288-4, or P07288-5 (entry version 197) or a fragment thereof;
   said Epidermal Growth Factor Receptor (EGFR) biomarker comprises an amino acid sequence having at least 70%, 80%, 90%, 95%, 99% or 100% sequence identity with the full-length of the amino acid sequence disclosed at UniProt accession no: Q504U8 (entry version 138) or a fragment thereof;
   said Neurofilament heavy polypeptide (NF-H) biomarker comprises an amino acid sequence having at least 70%, 80%, 90%, 95%, 99% or 100% sequence identity with the full-length of the amino acid sequence disclosed at UniProt accession no: P12036-1 or P12036-2 (entry version 177) or a fragment thereof;
   said Dopamine beta-hydroxylase (DBH) biomarker comprises an amino acid sequence having at least 70%, 80%, 90%, 95%, 99% or 100% sequence identity with the full-length of the amino acid sequence disclosed at UniProt accession no: P09172 (entry version 194) or a fragment thereof;
   said Fibulin-1C (Fib-1C) biomarker comprises an amino acid sequence having at least 70%, 80%, 90%, 95%, 99% or 100% sequence identity with the full-length of the amino acid sequence disclosed at UniProt accession no: P23142-1, P23142-2, P23142-3, or P23142-4 (entry version 217) or a fragment thereof;
   said Myoglobin biomarker comprises an amino acid sequence having at least 70%, 80%, 90%, 95%, 99% or 100% sequence identity with the full-length of the amino acid sequence disclosed at UniProt accession no: P02144 (entry version 178) or a fragment thereof;
   said Urokinase-type Plasminogen Activator (uPA) biomarker comprises an amino acid sequence having at least 70%, 80%, 90%, 95%, 99% or 100% sequence identity with the full-length of the amino acid sequence disclosed at UniProt accession no: P00749-1 or P00749-2 (entry version 230) or a fragment thereof;
   said Visceral adipose tissue-derived serpin A12 (Vaspin) biomarker comprises an amino acid sequence having at least 70%, 80%, 90%, 95%, 99% or 100% sequence identity with the full-length of the amino acid sequence disclosed at UniProt accession no: Q8IW75 (entry version 122) or a fragment thereof;
   said Interleukin-23 (IL-23) biomarker comprises an amino acid sequence having at least 70%, 80%, 90%, 95%, 99% or 100% sequence identity with the full-length of the amino acid sequence disclosed at UniProt accession no: Q9NPF7 (entry version 133) or a fragment thereof;
   said Alpha-2-Macroglobulin (A2Macro) biomarker comprises an amino acid sequence having at least 70%, 80%, 90%, 95%, 99% or 100% sequence identity with the full-length of the amino acid sequence disclosed at UniProt accession no: P01023 (entry version 206) or a fragment thereof;
   said Tissue Inhibitor of Metalloproteinases 2 (TIMP-2) biomarker comprises an amino acid sequence having at least 70%, 80%, 90%, 95%, 99% or 100% sequence identity with the full-length of the amino acid sequence disclosed at UniProt accession no: P16035 (entry version 187) or a fragment thereof;
   said Thyroid-Stimulating Hormone (TSH) biomarker comprises an amino acid sequence having at least 70%, 80%, 90%, 95%, 99% or 100% sequence identity with the full-length of the amino acid sequence disclosed at UniProt accession no: P01222-1 or P01222-2 (entry version 161) or a fragment thereof;
   said Tetranectin biomarker comprises an amino acid sequence having at least 70%, 80%, 90%, 95%, 99% or 100% sequence identity with the full-length of the amino acid sequence disclosed at UniProt accession no: P05452 (entry version 177) or a fragment thereof;
   said Myeloid Progenitor Inhibitory Factor 1 (MPIF-1) biomarker comprises an amino acid sequence having at least 70%, 80%, 90%, 95%, 99% or 100% sequence identity with the full-length of the amino acid sequence disclosed at UniProt accession no: P55773-1 or P55773-2 (entry version 162) or a fragment thereof;
   said Pancreatic Polypeptide (PPP) biomarker comprises an amino acid sequence having at least 70%, 80%, 90%, 95%, 99% or 100% sequence identity with the full-length of the amino acid sequence disclosed at UniProt accession no: P01298-1 or P01298-2 (entry version 175) or a fragment thereof;
   said Interleukin-7 (IL-7) biomarker comprises an amino acid sequence having at least 70%, 80%, 90%, 95%, 99% or 100% sequence identity with the full-length of the amino acid sequence disclosed at UniProt accession no: P13232-1, P13232-2, or P13232-3 (entry version 170) or a fragment thereof;
   said Pulmonary and Activation-Regulated Chemokine (PARC) biomarker comprises an amino acid sequence having at least 70%, 80%, 90%, 95%, 99% or 100% sequence identity with the full-length of the amino acid sequence disclosed at UniProt accession no: P55774 (entry version 152) or a fragment thereof;
   said Cancer Antigen 15-3 (CA-15-3) biomarker comprises an amino acid sequence having at least 70%, 80%, 90%, 95%, 99% or 100% sequence identity with the full-length of the amino acid sequence disclosed at UniProt accession no: P15941-1, P15941-2, P15941-3, P15941-4, P15941-5, P15941-6, P15941-7, P15941-8, P15941-9, P15941-10, P15941-11, P15941-12, P15941-13, P15941-14, P15941-15, P15941-16, or P15941-17 (entry version 204) or a fragment thereof;
   said Pepsinogen I (PGI) biomarker comprises an amino acid sequence having at least 70%, 80%, 90%, 95%, 99% or 100% sequence identity with the full-length of the amino acid sequence disclosed at UniProt accession no: P0DJD8 (entry version 50) or a fragment thereof;
   said Apolipoprotein E (Apo E) biomarker comprises an amino acid sequence having at least 70%, 80%, 90%, 95%, 99% or 100% sequence identity with the full-length of the amino acid sequence disclosed at UniProt accession no: P02649 (entry version 230) or a fragment thereof;
   said Apolipoprotein C-I (Apo C-I) biomarker comprises an amino acid sequence having at least 70%, 80%, 90%, 95%, 99% or 100% sequence identity with the full-length of the amino acid sequence disclosed at UniProt accession no: P02654 (entry version 175) or a fragment thereof;
   said Macrophage-Stimulating Protein (MSP) biomarker comprises an amino acid sequence having at least 70%, 80%, 90%, 95%, 99% or 100% sequence identity with the full-length of the amino acid sequence disclosed at UniProt accession no: P26927 (entry version 179) or a fragment thereof;
   said C-C motif chemokine 15 (CCL15) biomarker comprises an amino acid sequence having at least 70%, 80%, 90%, 95%, 99% or 100% sequence identity with the full-length of the amino acid sequence disclosed at UniProt accession no: Q16663 (entry version 161) or a fragment thereof;
   said Progranulin biomarker comprises an amino acid sequence having at least 70%, 80%, 90%, 95%, 99% or 100% sequence identity with the full-length of the amino acid sequence disclosed at UniProt accession no: P28799-1, P28799-2, or P28799-3 (entry version 197) or a fragment thereof;
   said Carcinoembryonic antigen-related cell adhesion molecule 1 (CEACAM1) biomarker comprises an amino acid sequence having at least 70%, 80%, 90%, 95%, 99% or 100% sequence identity with the full-length of the amino acid sequence disclosed at UniProt accession no: P13688-1, P13688-2, P13688-3, P13688-4, P13688-5, P13688-6, P13688-7, P13688-8, P13688-9, P13688-10, or P13688-11 (entry version 197) or a fragment thereof;
   said Vitamin D-Binding Protein (VDBP) biomarker comprises an amino acid sequence having at least 70%, 80%, 90%, 95%, 99% or 100% sequence identity with the full-length of the amino acid sequence disclosed at UniProt accession no: P02774-1, P02774-2, or P02774-3 (entry version 201) or a fragment thereof;
   said Carbonic anhydrase 9 (CA-9) biomarker comprises an amino acid sequence having at least 70%, 80%, 90%, 95%, 99% or 100% sequence identity with the full-length of the amino acid sequence disclosed at UniProt accession no: Q16790 (entry version 171) or a fragment thereof;
   said Lactate Dehydrogenase (LDH) biomarker comprises an amino acid sequence having at least 70%, 80%, 90%, 95%, 99% or 100% sequence identity with the full-length of the amino acid sequence disclosed at UniProt accession no: P00338-1, P00338-2, P00338-3, P00338-4, or P00338-5 (entry version 224) or a fragment thereof; and/or
   said Lactate Dehydrogenase (LDH) biomarker comprises an amino acid sequence having at least 70%, 80%, 90%, 95%, 99% or 100% sequence identity with the full-length of the amino acid sequence disclosed at UniProt accession no: P07195-1 (entry version 218) or a fragment thereof;
   or a nucleic acid (either DNA or RNA) having at least 70%, 80%, 90%, 95%, 99% or 100% sequence identity with the full-length nucleic acid sequence encoding said amino acid sequences.
113. A method according to any one of statement 101-112, wherein the method comprises determining the amount of at least 2, 3, 4, 5, 6, 7, 8, 9, 10, 11, 12, 13, 14, 15, 20 of said biomarkers in the subject, or sample derived from the subject.
114. A method according to any one of statements 104-113, wherein assessing the activity, expression, or amount of one or more biomarker in the subject, or in a sample derived from the subject, comprises:
   contacting a sample, or an extract from the sample, with at least 1, 2, 3, 4, 5, 6, 7, 8, 9, 10 or more reagents for detecting the activity, expression, or amount of one or more biomarkers, each of which is selective for a particular biomarker; and
   detecting and/or quantifying said reagents for detecting.
115. A method according to statement 114, wherein assessing the expression or amount of one or more biomarker in the subject, or in a sample derived from the subject, comprises:
   contacting a sample, or an extract from the sample, with at least 1, 2, 3, 4, 5, 6, 7, 8, 9, 10 or more specific binding members, each of which selectively binds to a respective biomarker protein or mRNA; and
   detecting and/or quantifying formation of complex formed by said specific binding member and said biomarker protein or mRNA.
116. A method according to statement 115 wherein the specific binding member comprises an antibody molecule or binding fragment thereof.
117. A method according to statement 115 or 116, wherein detecting formation of a complex formed by the specific binding member and the biomarker protein or mRNA is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.
118. A method according to any one of statements 115-117 wherein the expression or amount of the one or more biomarker is determined as the absolute amount of the respective biomarker protein or mRNA in the sample.
119. A method according to any one of statements 115-117 wherein the expression or amount of the one or more biomarker is determined as a relative amount of the respective biomarker protein or mRNA in the sample,
   optionally wherein the relative amount is determined relative to a reference protein or mRNA in the sample, the expression of which is not altered by NSCLC.
120. A method according to any one of statements 101-119, wherein the cancer-related outcome is susceptibility to treatment with an agent capable of inhibiting or reversing EMT or a chemotherapeutic agent.
121. A method according to statement 120, wherein the agent capable of inhibiting or reversing EMT or chemotherapeutic agent is an Axl inhibitor, Akt3 inhibitor, Slfn11 inhibitor, or PHGDH inhibitor.
122. A method according to statement 121, wherein the agent capable of inhibiting or reversing EMT or chemotherapeutic agent is an Axl inhibitor.
123. A method according to statement 122, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
124. A method according to statement 123, wherein the Axl inhibitor is BGB324/R428/bemcentinib.
125. A method according to statement 123, wherein the Axl inhibitor is an anti-Axl antibody described in WO2015/193428, WO2015/193430, WO2016/097370, or WO2016/166296.
126. A method according to statement 121, wherein the agent capable of inhibiting or reversing EMT or chemotherapeutic agent is an Akt3 inhibitor.
127. A method according to statement 126, wherein the Akt3 inhibitor is an Akt3 inhibitor disclosed in WO2016/102672.
128. A method according to any one of statements 120-127, wherein the agent capable of inhibiting or reversing EMT is administered as a single agent.
129. A method according to any one of statements 120-127, wherein the agent capable of inhibiting or reversing EMT is administered in combination with a further cancer treatment.
130. A method according to statement 129, wherein the further cancer treatment is selected from:
   (i) alkylating agents, including alkyl sulfonates such as busulfan;
   (ii) nitrogen mustards such as chlorambucil, cyclophosphamide, estramustine, ifosfamide, mechlorethamine, melphalan, and uramustine;
   (iii) ethyleneimine derivatives such as thiotepa;
   (iv) nitrosoureas such as carmustine, lomustine, and streptozocin;
   (v) triazenes such as dacarbazine, procarbazine, and temozolamide;
   (vi) platinum compounds such as cisplatin, carboplatin, oxaliplatin, satraplatin, and picoplatin onnaplatin, tetraplatin, sprioplatin, iproplatin, chloro(diethylenediamino)-platinum (II) chloride, dichloro(ethylenediamino)-platinum (II), diamino(2-ethylmalonato)platinum (II), (1,2-diaminocyclohexane)malonatoplatinum (II), (4-carboxyphthalo)-(1,2-diaminocyclohexane)platinum (II), (1,2-diaminocyclohexane)-(isocitrato)platinum (II), and (1,2-diaminocyclohexane)-cis-(pyruvato)platinum (II);
   (vii) antimetabolites, including antifolates such as methotrexate, permetrexed, raltitrexed, and trimetrexate;
   (viii) pyrimidine analogues such as azacitidine, capecitabine, cytarabine, edatrexate, floxuridine, fluorouracil, gemcitabine, and troxacitabine;
   (ix) purine analogues such as cladribine, chlorodeoxyadenosine, clofarabine, fludarabine, mercaptopurine, pentostatin, and thioguanine;
   (x) natural products, including antitumor antibiotics such as bleomycin, dactinomycin, mithramycin, mitomycin, mitoxantrone, porfiromycin;
   (xi) anthracyclines such as daunorubicin, doxorubicin, epirubicin, idarubicin, and valrubicin;
   (xii) mitotic inhibitors such as the vinca alkaloids vinblastine, vinvesir, vincristine, vindesine, and vinorelbine;
   (xiii) enzymes such as L-asparaginase and PEG-L-asparaginase;
   (xiv) microtubule polymer stabilizers such as the taxanes paclitaxel and docetaxel;
   (xv) topoisomerase I inhibitors such as the camptothecins irinotecan and topotecan;
   (xvi) topoisomerase II inhibitors such as podophyllotoxin, amsacrine, etoposide, teniposide, losoxantrone and actinomycin;
   (xvii) hormones and hormone antagonists, including
      androgens such as fluoxymesterone and testolactone;
      antiandrogens such as bicalutamide, cyproterone, flutamide, and nilutamide;
      corticosteroids such as dexamethasone and prednisone;
      aromatase inhibitors such as aminoglutethimide, anastrozole, exemestane, formestane, and letrozole;
      estrogens such as diethylstilbestrol;
      antiestrogens such as fulvestrant, raloxifene, tamoxifen, and toremifine;
      luteinising hormone-releasing hormone (LHRH) agonists and antagonists such as abarelix, buserelin, goserelin, leuprolide, histrelin, desorelin, nafarelin acetate and triptorelin;
      progestins such as medroxyprogesterone acetate and megestrol acetate; and
      thyroid hormones such as levothyroxine and liothyronine;
   (xviii) PKB pathway inhibitors, including perifosine, enzastaurin hydrochloride, and triciribine;
   (xix) P13K inhibitors such as semaphore and SF1126, and MTOR inhibitors such as rapamycin and analogues;
   (xx) CDK inhibitors, including seliciclib, alvocidib, and 7-hydroxystaurosporine;
   (xxi) COX-2 inhibitors, including celecoxib;
   (xxii) HDAC inhibitiors, including trichostatin A, suberoylanilide hydroxamic acid, and chlamydocin;
   (xxiii) DNA methylase inhibitors, including temozolomide, and miscellaneous agents, including altretamine, arsenic trioxide, thalidomide, lenalidomide, gallium nitrate, levamisole, mitotane, hydroxyurea, octreotide, procarbazine, suramin, photodynamic compounds such as methoxsalen and sodium porfimer;
   (xxiv) proteasome inhibitors such as bortezomib;
   (xxv) molecular targeted therapy agents including: functional therapeutic agents, including gene therapy agents and antisense therapy agents;
   (xxvi) tyrosine kinase inhibitors such as erlotinib hydrochloride, gefitinib, imatinib mesylate, and semaxanib;
   (xxvii) Raf inhibitors such as sorafenib, and gene expression modulators such as the retinoids and rexinoids, for example adapalene, bexarotene, trans-retinoic acid, 9-cis-retinoic acid, and N-(4-hydroxyphenyl)retinamide;
   (xxviii) phenotype-directed therapy agents, including monoclonal antibodies such as pembrolizumab, alemtuzumab, bevacizumab, cetuximab, ibritumomab tiuxetan, rituximab, and trastuzumab, immunotoxins such as gemtuzumab ozogamicin, radioimmunoconjugates such as I-tositumobab;
   (xxix) cancer vaccines;
   (xxx) biologic therapy agents including: interferons such as interferon-[alpha]2a and interferon-[alpha]2b, and interleukins such as aldesleukin, denileukin diftitox, and oprelvekin;
   (xxxi) anticancer therapies involving the use of protective or adjunctive agents, including: cytoprotective agents such as amifostine, and dexrazoxane, phosphonates such as pamidronate and zoledronic acid, and stimulating factors such as epoetin, darbeopetin, filgrastim, PEG-filgrastim, and sargramostim;
   (xxxii) Axl inhibitors such as 1-(6,7-dihydro-5H-benzo[6,7]cyclohepta[1,2-c]pyridazin-3-yl)-N3-((7-(S)-pyrrolidin-1-yl)-6,7,8,9-tetrahydro-5H-benzo[7]annulene-2-yl)-1H-1,2,4-triazole-3,5-diamine; and/or
   (xxxiii) further combination chemotherapeutic regimens, such as combinations of carboplatin/paclitaxel, capecitabine/docetaxel, fluorauracil/levamisole, fluorauracil/leucovorin, methotrexate/leucovorin, and trastuzumab/paclitaxel, alone or in further combination with carboplatin.
131. A method according to statement 129, wherein the further cancer treatment is:
   (i) a PD-1 inhibitor or PD-L1 inhibitor;
   (ii) a monoclonal antibody such as pembrolizumab, alemtuzumab, bevacizumab, cetuximab, ibritumomab, tiuxetan, or rituximab;
   (iii) a microtubule polymer stabilizer such as the taxanes paclitaxel and docetaxel; and/or
   (iv) a tyrosine kinase inhibitor such as erlotinib hydrochloride, gefitinib, imatinib mesylate, and semaxanib.
132. A method according to statement 131, wherein the further cancer treatment is pembrolizumab.
133. A method according to statement 131, wherein the further cancer treatment is docetaxel.
134. A method according to statement 131, wherein the further cancer treatment is erlotinib.
135. A method according to any one of statements 101-134, wherein the subject is mammalian, optionally wherein the subject is human.
136. A method according to any one of statements 101-135, wherein the predictive method is performed before the subject receives a treatment or course of treatment for NSCLC.
137. A method according to any one of statement 101-136, wherein the predictive method is performed during or after a treatment or course of treatment for NSCLC.
138. A method according to any one of statements 101-135 or 137, wherein the subject has previously been treated with an agent capable of inhibiting or reversing EMT or a chemotherapeutic agent.
139. A method according to statement 138, wherein the subject has previously been treated with an agent selected from: a PHGDH inhibitor, a Slfn11 inhibitor, an Axl inhibitor, or an Akt3 inhibitor.
140. A method according to any one of statements 138-139, wherein the subject has previously been treated with an agent that is different to the agent capable of inhibiting or reversing EMT or chemotherapeutic agent for which the subject's susceptibility is being predicted.
141. A method according to any one of statements 101-136, wherein the subject has not previously been treated with an agent capable of inhibiting or reversing EMT or a chemotherapeutic agent.
142. A method according to statement 141, wherein the subject has not previously been treated with an Axl inhibitor.
143. A method according to statement 141, wherein the subject has not previously been treated with an Akt3 inhibitor.
144. A method according to any one of statements 101-143, wherein the sample is selected from: blood, serum, plasma, tissue biopsy, culture supernatant, or urine.
145. A method according to statement 144, wherein the sample is a blood, serum, or plasma sample.
146. A method according to statement 145, wherein the sample is a serum sample.
147. A method according to any one of statements 104-146, wherein the method further comprises a subsequent step of assessing the activity, expression, or amount of one or more biomarker in the subject, in which a sample profile is obtained after contacting the subject with an agent capable of inhibiting or reversing EMT or a chemotherapeutic agent.
148. A method according to statement 103, wherein the sample profile is obtained after contacting the subject with an agent capable of inhibiting or reversing EMT or a chemotherapeutic agent.
149. A method according to statement 147 or 148, wherein the control profile is obtained from the same subject prior to contacting said subject with the agent capable of inhibiting or reversing EMT or a chemotherapeutic agent.
150. A method according to statement 149, wherein an increase in the activity, expression, or amount of one or more biomarker in the sample profile as compared with the control profile is indicative of higher susceptibility to treatment with an agent capable of inhibiting or reversing EMT or a chemotherapeutic agent.
151. A method according to statement 149, wherein a decrease in the activity, expression, or amount of one or more biomarker in the sample profile as compared with the control profile is indicative of higher susceptibility to treatment with an agent capable of inhibiting or reversing EMT or a chemotherapeutic agent.
152. A method according to statement 149, wherein an increase in the activity, expression, or amount of one or more biomarker in the sample profile as compared with the control profile is indicative of lower susceptibility to treatment with an agent capable of inhibiting or reversing EMT or a chemotherapeutic agent.
153. A method according to statement 149, wherein a decrease in the activity, expression, or amount of one or more biomarker in the sample profile as compared with the control profile is indicative of lower susceptibility to treatment with an agent capable of inhibiting or reversing EMT or a chemotherapeutic agent.
154. A method according to statement 150, wherein the one or more biomarker includes at least Axl.
155. A method according to any one of statements 147-154, wherein assessing the activity, expression, or amount of one or more biomarker in the subject, or in a sample derived from the subject, comprises:
   contacting a sample, or an extract from the sample, with at least 1, 2, 3, 4, 5, 6, 7, 8, 9, 10 or more reagents for detecting the activity, expression, or amount of one or more biomarkers, each of which is selective for a particular biomarker; and
   detecting and/or quantifying said reagents for detecting.
156. A method according to statement 155, wherein assessing the expression or amount of one or more biomarker in the subject, or in a sample derived from the subject, comprises:
   contacting a sample, or an extract from the sample, with at least 1, 2, 3, 4, 5, 6, 7, 8, 9, 10 or more specific binding members, each of which selectively binds to a respective biomarker protein or mRNA; and
   detecting and/or quantifying formation of a complex formed by said specific binding member and said biomarker protein or mRNA.
157. A method according to statement 156, wherein the specific binding member comprises an antibody molecule or binding fragment thereof.
158. A method according to statement 156 or 157, wherein detecting formation of a complex formed by the specific binding member and the biomarker protein or mRNA is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.
159. A method according to any one of statements 156-158 wherein the expression or amount of the one or more biomarker is determined as the absolute amount of the respective biomarker protein or mRNA in the sample.
160. A method according to any one of statements 156-158 wherein the expression or amount of the one or more biomarker is determined as a relative amount of the respective biomarker protein or mRNA in the sample,
   optionally wherein the relative amount is determined relative to a reference protein or mRNA in the sample, the expression of which is not altered by NSCLC.
161. A method according to any one of statements 148-160, wherein the agent capable of inhibiting or reversing EMT or chemotherapeutic agent is an Axl inhibitor, Akt3 inhibitor, Slfn11 inhibitor, or PHGDH inhibitor.
162. A method according to statement 161, wherein the agent capable of inhibiting or reversing EMT or chemotherapeutic agent is an Axl inhibitor.
163. A method according to statement 162, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
164. A method according to statement 163, wherein the Axl inhibitor is BGB324/R428/bemcentinib.
165. A method according to statement 163, wherein the Axl inhibitor is an anti-Axl antibody described in WO2015/193428, WO2015/193430, WO2016/097370, or WO2016/166296.
166. A method according to statement 161, wherein the agent capable of inhibiting or reversing EMT or chemotherapeutic agent is an Akt3 inhibitor.
167. A method according to statement 166, wherein the Akt3 inhibitor is an Akt3 inhibitor disclosed in WO2016/102672.
168. A method according to any one of statements 161-167, wherein the agent capable of inhibiting or reversing EMT is administered as a single agent.
169. A method according to any one of statements 161-167, wherein the agent capable of inhibiting or reversing EMT is administered in combination with a further cancer treatment.
170. A method according to statement 169, wherein the further cancer treatment is selected from the further cancer treatments listed in statement 130.
171. A method according to statement 169, wherein the further cancer treatment is:
   (i) a PD-1 inhibitor or PD-L1 inhibitor;
   (ii) a monoclonal antibody such as pembrolizumab, alemtuzumab, bevacizumab, cetuximab, ibritumomab, tiuxetan, or rituximab;
   (iii) a microtubule polymer stabilizer such as the taxanes paclitaxel and docetaxel; and/or
   (iv) a tyrosine kinase inhibitor such as erlotinib hydrochloride, gefitinib, imatinib mesylate, and semaxanib.
172. A method according to statement 171, wherein the further cancer treatment is pembrolizumab.
173. A method according to statement 171, wherein the further cancer treatment is docetaxel.
174. A method according to statement 171, wherein the further cancer treatment is erlotinib.
175. A method according to any one of statements 148-174, wherein the subject is mammalian, optionally wherein the subject is human.
176. A method according to any one of statements 148-175, wherein the subject was selected for treatment with the agent capable of inhibiting or reversing EMT or a chemotherapeutic agent, using a predictive method according to any one of statements 101-147.
177. A method according to any one of statements 148-176, wherein the sample is selected from: blood, serum, plasma, tissue biopsy, culture supernatant, or urine.
178. A method according to statement 177, wherein the sample is a blood, serum, or plasma sample.
179. A method according to statement 178, wherein the sample is a serum sample.
180. A method according to any one of statements 1-179, wherein the method is performed in vitro or ex vivo.
201. A method of selecting subjects having, suspected of having, or diagnosed with non-small cell lung cancer (NSCLC) for treatment with an agent capable of inhibiting or reversing EMT or a chemotherapeutic agent, the method comprising:
   identifying subjects susceptible to treatment with an agent capable of inhibiting or reversing EMT or a chemotherapeutic agent using a method according to any one of statements 101-180; and
   selecting thus identified subjects for treatment.
202. A method of selecting subjects having, suspected of having, or diagnosed with non-small cell lung cancer (NSCLC) for continued treatment with an agent capable of inhibiting or reversing EMT or a chemotherapeutic agent, the method comprising:
   identifying subjects susceptible to treatment with an agent capable of inhibiting or reversing EMT or a chemotherapeutic agent using a method according to any one of statements 148-180; and
   selecting thus identified subjects for treatment.
203. A method according to any one of statements 201-202, wherein treatment comprises administering to the subject a therapeutically effective amount of an agent capable of inhibiting or reversing EMT or a chemotherapeutic agent.
204. A method according to statement 203, wherein the treatment comprises an Axl inhibitor, Akt3 inhibitor, Slfn11 inhibitor, or PHGDH inhibitor.
205. A method according to statement 204, wherein the treatment comprises an Axl inhibitor.
206. A method according to statement 205, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
207. A method according to statement 206, wherein the Axl inhibitor is BGB324/R428/bemcentinib.
208. A method according to statement 206, wherein the Axl inhibitor is an anti-Axl antibody described in WO2015/193428, WO2015/193430, WO2016/097370, or WO2016/166296.
209. A method according to statement 204, wherein the treatment comprises an Akt3 inhibitor.
210. A method according to statement 209, wherein the Akt3 inhibitor is an Akt3 inhibitor disclosed in WO2016/102672.
211. A method according to any one of statements 203-210, wherein the treatment is administered as a single agent.
212. A method according to any one of statements 203-211, wherein the treatment is administered in combination with a further cancer treatment.
213. A method according to statement 212, wherein the further cancer treatment is selected from the further cancer treatments listed in statement 130.
214. A method according to statement 212, wherein the further cancer treatment is:
   (i) a PD-1 inhibitor or PD-L1 inhibitor;
   (ii) a monoclonal antibody such as pembrolizumab, alemtuzumab, bevacizumab, cetuximab, ibritumomab, tiuxetan, or rituximab;
   (iii) a microtubule polymer stabilizer such as the taxanes paclitaxel and docetaxel; and/or
   (iv) a tyrosine kinase inhibitor such as erlotinib hydrochloride, gefitinib, imatinib mesylate, and semaxanib.
215. A method according to statement 214, wherein the further cancer treatment is pembrolizumab.
216. A method according to statement 214, wherein the further cancer treatment is docetaxel.
217. A method according to statement 214, wherein the further cancer treatment is erlotinib.
301. A diagnostic kit comprising 1, 2, 3, 4, 5, 6, 7, 8, 9, 10 or more reagents for detecting the activity, expression, or amount of one or more biomarkers, each of which is selective for a particular biomarker selected from the group consisting of: Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, TIMP-2, TSH, MPIF-1, PPP, IL-7, PARC, CA-15-3, PGI, Apo E, Apo C-I, MSP, CCL15, Progranulin, CEACAM1, VDBP, CA-9, and/or LDH.
302. A test device comprising 1, 2, 3, 4, 5, 6, 7, 8, 9, 10 or more reagents for detecting the activity, expression, or amount of one or more biomarkers, each of which is selective for a particular biomarker selected from the group consisting of: Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, TIMP-2, TSH, MPIF-1, PPP, IL-7, PARC, CA-15-3, PGI, Apo E, Apo C-I, MSP, CCL15, Progranulin, CEACAM1, VDBP, CA-9, and/or LDH.
303. A diagnostic kit or test device according to any one of statements 301 or 302, comprising:
   1, 2, 3, 4, 5, 6, 7, 8, 9, 10 or more specific binding members, each of which selectively binds to a biomarker selected from the group consisting of: Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, TIMP-2, TSH, MPIF-1, PPP, IL-7, PARC, CA-15-3, PGI, Apo E, Apo C-I, MSP, CCL15, Progranulin, CEACAM1, VDBP, CA-9, and/or LDH; and
   one or more reagents for detecting said one or more specific binding members or one or more reagents for detecting and/or quantifying formation of a complex formed by said specific binding member and said biomarker.
304. A diagnostic kit or test device according to any one of statements 301-303, wherein each reagent for detecting or specific binding member is selective for a biomarker selected from the group:
   (i) Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, TIMP-2, and/or LDH;
   (ii) Axl, TSH, MPIF-1, PPP, Vaspin, IL-7, PARC, CA-15-3, and/or LDH; or
   (iii) Axl, PGI, Apo E, Apo C-I, MSP, CCL15, Progranulin, CEACAM1, VDBP, CA-9, and/or LDH.
305. A diagnostic kit or test device according to statement 303 or 304, wherein the specific binding member comprises an antibody molecule or binding fragment thereof.
306. A diagnostic kit or test device according to any of statements 303-305, wherein detecting formation of a complex formed by the specific binding member and the biomarker is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.
307. A diagnostic kit or test device according to any one of statements 301-306, wherein the kit or test device comprises no more than 15, 20, 25, 30, 40, or 50 reagents for detecting.
308. A diagnostic kit or test device according to any one of statements 301-307 for use in a method of predicting a cancer-related outcome in a subject.
309. A diagnostic kit for use, or test device for use, according to statement 308, wherein the cancer-related outcome is susceptibility to treatment with an agent capable of inhibiting or reversing EMT or a chemotherapeutic agent.
310. A diagnostic kit for use, or test device for use, according to statement 309, wherein the agent capable of inhibiting or reversing EMT or chemotherapeutic agent is an Axl inhibitor, Akt3 inhibitor, Slfn11 inhibitor, or PHGDH inhibitor.
311. A diagnostic kit for use, or test device for use, according to statement 309, wherein the agent capable of inhibiting or reversing EMT or chemotherapeutic agent is an Axl inhibitor.
312. A diagnostic kit for use, or test device for use, according to statement 311, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
313. A diagnostic kit for use, or test device for use, according to statement 312, wherein the Axl inhibitor is BGB324/R428/bemcentinib.
314. A diagnostic kit for use, or test device for use, according to statement 312, wherein the Axl inhibitor is an anti-Axl antibody described in WO2015/193428, WO2015/193430, WO2016/097370, or WO2016/166296.
315. A diagnostic kit for use, or test device for use, according to statement 309, wherein the agent capable of inhibiting or reversing EMT or chemotherapeutic agent is an Akt3 inhibitor.
316. A diagnostic kit for use, or test device for use, according to any one of statements 308-311, wherein the subject has, is suspected of having, or has been diagnosed with non-small cell lung cancer (NSCLC).
317. A diagnostic kit for use, or test device for use, according to any one of statements 308-316, wherein the method is a method according to any one of statements 101-180.
318. Use of a reagent for detecting the activity, expression, or amount of a biomarker selected from the group consisting of: Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, TIMP-2, TSH, MPIF-1, PPP, IL-7, PARC, CA-15-3, PGI, Apo E, Apo C-I, MSP, CCL15, Progranulin, CEACAM1, VDBP, CA-9, and/or LDH, in a method of predicting a cancer-related outcome in a subject.
319. Use of 1, 2, 3, 4, 5, 6, 7, 8, 9, 10 or more reagents for detecting the activity, expression, or amount a biomarker, each of which is selective for a particular biomarker selected from the group consisting of: Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, TIMP-2, TSH, MPIF-1, PPP, IL-7, PARC, CA-15-3, PGI, Apo E, Apo C-I, MSP, CCL15, Progranulin, CEACAM1, VDBP, CA-9, and/or LDH, in a method of manufacture of a diagnostic kit or test device for use in a method of predicting a cancer-related outcome in a subject.
320. A use according to any one of statements 318 or 319, wherein each reagent for detecting is selective for a biomarker selected from the group:
   (i) Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, TIMP-2, and/or LDH;
   (ii) Axl, TSH, MPIF-1, PPP, Vaspin, IL-7, PARC, CA-15-3, and/or LDH; or
   (iii) Axl, PGI, Apo E, Apo C-I, MSP, CCL15, Progranulin, CEACAM1, VDBP, CA-9, and/or LDH.
321. A use according to any one of statements 318-320, wherein the reagent for detecting comprises a specific binding member selective for a particular biomarker.
322. A use according to any one of statement 318-321, wherein the method of predicting a cancer-related outcome in a subject is a method according to any one of statements 101-180.
401. Use of one or more of: Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, TIMP-2, TSH, MPIF-1, PPP, IL-7, PARC, CA-15-3, PGI, Apo E, Apo C-I, MSP, CCL15, Progranulin, CEACAM1, VDBP, CA-9, and/or LDH, as a biomarker for determining if a subject is susceptible to treatment with an agent capable of inhibiting or reversing EMT or a chemotherapeutic agent.
402. A use according to statement 401, wherein:
   (i) a higher activity, expression, or amount of one or more of said biomarkers in the subject, or sample derived from the subject, compared with a control profile for the respective biomarker is indicative of susceptibility to treatment with an agent capable of inhibiting or reversing EMT or a chemotherapeutic agent; or
   (ii) a lower activity, expression, or amount of one or more of said biomarkers in the subject, or sample derived from the subject, compared with a control profile for the respective biomarker is indicative of susceptibility to treatment with an agent capable of inhibiting or reversing EMT or a chemotherapeutic agent.
403. A use according to statement 401, wherein:
   (i) an increase in the activity, expression, or amount of one or more biomarker relative to a control profile for the respective biomarker after contacting the subject with a cancer therapeutic agent is indicative of increased susceptibility to treatment with an agent capable of inhibiting or reversing EMT or a chemotherapeutic agent; or
   (ii) a decrease in the activity, expression, or amount of one or more biomarker relative to a control profile for the respective biomarker after contacting the subject with a cancer therapeutic agent is indicative of increased susceptibility to treatment with an agent capable of inhibiting or reversing EMT or a chemotherapeutic agent.
404. A use according to any one of statement 401-403, wherein the one or more biomarker is selected from the group consisting of:
   (i) Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, TIMP-2, and/or LDH;
   (ii) Axl, TSH, MPIF-1, PPP, Vaspin, IL-7, PARC, CA-15-3, and/or LDH; or
   (iii) Axl, PGI, Apo E, Apo C-I, MSP, CCL15, Progranulin, CEACAM1, VDBP, CA-9, and/or LDH.
405. A use according to any one of statements 401-404, wherein the use is as a biomarker in a method according to any one of statements 101-180
501. A method of treating a subject having, suspected of having, or diagnosed with non-small cell lung cancer (NSCLC), the method comprising:
   identifying a subject susceptible to treatment using a method according to any one of statements 101-180; and
   administering to the subject a therapeutically effective amount of an agent capable of inhibiting or reversing EMT or a chemotherapeutic agent.
502. A method of treating a subject having, suspected of having, or diagnosed with non-small cell lung cancer (NSCLC), the method comprising:
   (a) obtaining a sample from the subject;
   (b) determining if the subject is susceptible to treatment with an agent capable of inhibiting or reversing EMT or a chemotherapeutic agent using a method according to any one of statements 101-180;
   (c) administering to the subject a therapeutically effective amount of an agent capable of inhibiting or reversing EMT or a chemotherapeutic agent.
503. A method of treating a subject having, suspected of having, or diagnosed with non-small cell lung cancer (NSCLC), the method comprising administering a therapeutically effective amount of an agent capable of inhibiting or reversing EMT or chemotherapeutic agent to a subject identified as susceptible to said treatment using a method according to any one of statements 101-180.
504. A method of treating a subject having, suspected of having, or diagnosed with non-small cell lung cancer (NSCLC), the method comprising:
   administering to the subject a therapeutically effective amount of an agent capable of inhibiting or reversing EMT or a chemotherapeutic agent; and
   determining if the subject is susceptible to said treatment using a method according to any one of statements 148-180.
505. A method according to statement 504, wherein the method comprises administering to the subject one or more further therapeutically effective amounts of an agent capable of inhibiting or reversing EMT or a chemotherapeutic agent provided that the subject is identified as susceptible to said treatment.
506. An agent capable of inhibiting or reversing EMT or chemotherapeutic agent for use in a method of treating a subject having, suspected of having, or diagnosed with non-small cell lung cancer (NSCLC),
   wherein the method is a method according to any one of statements 501-505.
507. Use of an agent capable of inhibiting or reversing EMT or chemotherapeutic agent for use in a method of manufacture of a medicament for use in a method of treating a subject having, suspected of having, or diagnosed with non-small cell lung cancer (NSCLC),
   wherein the method of treatment is a method according to any one of statements 501-505.
508. A method, agent for use, or use according to any one of statements 501-507, wherein the agent capable of inhibiting or reversing EMT or chemotherapeutic agent comprises an Axl inhibitor, Akt3 inhibitor, Slfn11 inhibitor, or PHGDH inhibitor.
509. A method, agent for use, or use according to statement 508, wherein the agent capable of inhibiting or reversing EMT or chemotherapeutic agent comprises an Axl inhibitor.
510. A method, agent for use, or use according to statement 509, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
511. A method, agent for use, or use according to statement 510, wherein the Axl inhibitor is BGB324/R428/bemcentinib.
512. A method, agent for use, or use according to statement 510, wherein the Axl inhibitor is an anti-Axl antibody described in WO2015/193428, WO2015/193430, WO2016/097370, or WO2016/166296.
512. A method, agent for use, or use according to statement 508, wherein the agent capable of inhibiting or reversing EMT or chemotherapeutic agent comprises an Akt3 inhibitor.
513. A method, agent for use, or use according to statement 512, wherein the Akt3 inhibitor is an Akt3 inhibitor disclosed in WO2016/102672.
514. A method, agent for use, or use according to any one of statements 501-513, wherein the agent capable of inhibiting or reversing EMT or chemotherapeutic agent is administered as a single agent.
515. A method, agent for use, or use according to any one of statements 501-513, wherein the agent capable of inhibiting or reversing EMT or chemotherapeutic agent is administered in combination with a further cancer treatment.
516. A method, agent for use, or use according to statement 515, wherein the further cancer treatment is selected from the further cancer treatments listed in statement 130.
517. A method, agent for use, or use according to statement 515, wherein the further cancer treatment is:
   (i) a PD-1 inhibitor or PD-L1 inhibitor;
   (ii) a monoclonal antibody such as pembrolizumab, alemtuzumab, bevacizumab, cetuximab, ibritumomab, tiuxetan, or rituximab;
   (iii) a microtubule polymer stabilizer such as the taxanes paclitaxel and docetaxel; and/or
   (iv) a tyrosine kinase inhibitor such as erlotinib hydrochloride, gefitinib, imatinib mesylate, and semaxanib.
518. A method, agent for use, or use according to statement 517, wherein the further cancer treatment is pembrolizumab.
519. A method, agent for use, or use according to statement 517, wherein the further cancer treatment is docetaxel.
520. A method, agent for use, or use according to statement 517, wherein the further cancer treatment is erlotinib.

Facet 6a: Non-Small Cell Lung Cancer (NSCLC)

Non-small cell lung cancers (NSCLC) are any type of epithelial lung cancer other than small cell lung carcinoma (SCLC), and make up the majority of all lung cancers. Non-small cell lung carcinomas are grouped together because their prognosis and management are similar—the three main histologic classes are: squamous cell lung carcinoma, adenocarcinoma, and large cell lung carcinoma. Adenocarcinoma is the most common histologic type of lung carcinoma. Non-small cell lung cancers are often treated with cisplatin or carboplatin, in combination with gemcitabine, paclitaxel, docetaxel, etoposide or vinorelbine. There remains a need for targeted therapies for NSCLC (Herbst et al, 2018; Zappa et al, 2016).

Accordingly, robust biomarkers for predicting cancer-related outcomes in a subject having NSCLC, for example susceptibility to treatment with an agent capable of inhibiting or reversing EMT or a chemotherapeutic agent, would be useful in identifying subjects who are most likely to respond to or benefit from treatment with a particular agent/regimen.

A “responder” subject as described herein is a subject who is likely to, or who does, respond to treatment (or benefit from treatment) with an agent capable of inhibiting or reversing EMT or a chemotherapeutic agent, for example the small molecule Axl kinase inhibitor BGB324/R428/bemcentinib when administered either as a single agent or in combination with another cancer treatment.

Correspondingly, a “non-responder” subject as described herein is a subject who is not likely to, or who does not, respond to treatment (or benefit from treatment) with an agent capable of inhibiting or reversing EMT or a chemotherapeutic agent, for example the small molecule Axl kinase inhibitor BGB324/R428/bemcentinib when administered either as a single agent or in combination with another cancer treatment.

To “respond to treatment” or “benefit from treatment” as used herein means to experience an overall clinical benefit from the treatment. This overall clinical benefit can be any of: prolonged survival, partial or complete disease remission, slowing or absence of disease progression, tumour shrinkage (for example, a reduction in tumour volume of 5, 10, 20, 30, 40% or more), reduction in tumour burden (for example, a reduction in tumour burden of 5, 10, 20, 30, 40% or more), slowing or absence of tumour enlargement, slowing or absence of increase in tumour burden, improved quality of life, progression-free survival, overall survival, or any other positive patient outcome. Suitable methods for determining tumour volume are well known to the skilled person, for example, using: computed tomography (CT), or magnetic resonance imaging (MRI) imaging technologies; X-ray imaging, for example, mammography; ultrasound imaging; nuclear imaging, for example positron emission tomography (PET), PET/CT scans, bone scans, gallium scans, or metaiodobenzylguanidine (MIBG) scans; bioluminescence imaging (BLI); fluorescence imaging (FLI); BD ToF (infrared-based 3D Time-of-Flight camera) imaging.

Biomarkers

The term “marker” or “biomarker” is used herein to refer to a gene or protein whose expression in a subject or sample derived from a subject is altered or modulated, for example, up or down regulated, in cancer. Where the biomarker is a protein, modulation or alteration of expression encompasses modulation through different post-translational modifications.

In accordance with any aspect of this facet of the present disclosure the biomarkers described herein may be defined as follows, wherein:
an Axl biomarker comprises an amino acid sequence having at least 70%, 80%, 90%, 95%, 99% or 100% sequence identity with the full-length of the amino acid sequence disclosed at UniProt accession no: P30530-1 or P30530-2 (entry version 196) or a fragment thereof;
a N-terminal prohormone of brain natriuretic peptide (NT proBNP) biomarker comprises an amino acid sequence having at least 70%, 80%, 90%, 95%, 99% or 100% sequence identity with the full-length of the amino acid sequence disclosed at UniProt accession no: P16860-1 (entry version 186) or a fragment thereof;
a Endostatin biomarker comprises an amino acid sequence having at least 70%, 80%, 90%, 95%, 99% or 100% sequence identity with the full-length of the amino acid sequence disclosed at UniProt accession no: P39059-1 (entry version 170) or a fragment thereof;
a Insulin-like Growth Factor-Binding Protein 2 (IGFBP-2) biomarker comprises an amino acid sequence having at least 70%, 80%, 90%, 95%, 99% or 100% sequence identity with the full-length of the amino acid sequence disclosed at UniProt accession no: P18065-1 (entry version 193) or a fragment thereof;
a Vascular endothelial growth factor D (VEGF-D) biomarker comprises an amino acid sequence having at least 70%, 80%, 90%, 95%, 99% or 100% sequence identity with the full-length of the amino acid sequence disclosed at UniProt accession no: 043915-1 (entry version 159) or a fragment thereof;
a Growth/differentiation factor 15 (GDF-15) comprises an amino acid sequence having at least 70%, 80%, 90%, 95%, 99% or 100% sequence identity with the full-length of the amino acid sequence disclosed at UniProt accession no: Q99988-1 (entry version 166) or a fragment thereof;
a Beta Amyloid 1-40 (AB-40) biomarker comprises an amino acid sequence having at least 70%, 80%, 90%, 95%, 99% or 100% sequence identity with the full-length of the amino acid sequence disclosed at UniProt accession no: P05067-1, P05067-2, P05067-3, P05067-4, P05067-5, P05067-6, P05067-7, P05067-8, P05067-9, P05067-10, or P05067-11 (entry version 271) or a fragment thereof;
a Antileukoproteinase (ALP) biomarker comprises an amino acid sequence having at least 70%, 80%, 90%, 95%, 99% or 100% sequence identity with the full-length of the amino acid sequence disclosed at UniProt accession no: P03973 (entry version 184) or a fragment thereof;
a Monocyte Chemotactic Protein 3 (MCP-3) comprises an amino acid sequence having at least 70%, 80%, 90%, 95%, 99% or 100% sequence identity with the full-length of the amino acid sequence disclosed at UniProt accession no: P80098-1 (entry version 169) or a fragment thereof;
a Superoxide Dismutase 1, soluble (SOD-1) biomarker comprises an amino acid sequence having at least 70%, 80%, 90%, 95%, 99% or 100% sequence identity with the full-length of the amino acid sequence disclosed at UniProt accession no: P00441-1 (entry version 240) or a fragment thereof;
a Bone morphogenetic protein 9 (BMP-9) biomarker comprises an amino acid sequence having at least 70%, 80%, 90%, 95%, 99% or 100% sequence identity with the full-length of the amino acid sequence disclosed at UniProt accession no: Q9UK05-1 (entry version 162) or a fragment thereof;
a Interleukin-8 (IL-8) biomarker comprises an amino acid sequence having at least 70%, 80%, 90%, 95%, 99% or 100% sequence identity with the full-length of the amino acid sequence disclosed at UniProt accession no: P10145 (entry version 210) or a fragment thereof;
a Macrophage Inflammatory Protein-1 beta (MIP-1 beta) biomarker comprises an amino acid sequence having at least 70%, 80%, 90%, 95%, 99% or 100% sequence identity with the full-length of the amino acid sequence disclosed at UniProt accession no: P13236 (entry version 187) or a fragment thereof;
a Receptor for advanced glycosylation end products (RAGE) biomarker comprises an amino acid sequence having at least 70%, 80%, 90%, 95%, 99% or 100% sequence identity with the full-length of the amino acid sequence disclosed at UniProt accession no: Q15109-1, Q15109-2, Q15109-3, Q15109-4, Q15109-5, Q15109-6, Q15109-7, Q15109-8, Q15109-9, Q15109-10 (entry version 189) or a fragment thereof;
a Myeloperoxidase (MPO) biomarker comprises an amino acid sequence having at least 70%, 80%, 90%, 95%, 99% or 100% sequence identity with the full-length of the amino acid sequence disclosed at UniProt accession no: P05164-1, P05164-2, or P05164-3 (entry version 217) or a fragment thereof;
a Chromogranin-A (CgA) biomarker comprises an amino acid sequence having at least 70%, 80%, 90%, 95%, 99% or 100% sequence identity with the full-length of the amino acid sequence disclosed at UniProt accession no: P10645-1 (entry version 200) or a fragment thereof;
a HE4 biomarker comprises an amino acid sequence having at least 70%, 80%, 90%, 95%, 99% or 100% sequence identity with the full-length of the amino acid sequence disclosed at UniProt accession no: Q14508-1, Q14508-2, Q14508-3, Q14508-4, or Q14508-5 (entry version 169) or a fragment thereof;
a Adrenomedullin (ADM) biomarker comprises an amino acid sequence having at least 70%, 80%, 90%, 95%, 99% or 100% sequence identity with the full-length of the amino acid sequence disclosed at UniProt accession no: P35318-1 (entry version 174) or a fragment thereof;
a Beta-2-Microglobulin (B2M) biomarker comprises an amino acid sequence having at least 70%, 80%, 90%, 95%, 99% or 100% sequence identity with the full-length of the amino acid sequence disclosed at UniProt accession no: P61769-1 (entry version 181) or a fragment thereof;
a Vitamin D-Binding Protein (VDBP) biomarker comprises an amino acid sequence having at least 70%, 80%, 90%, 95%, 99% or 100% sequence identity with the full-length of the amino acid sequence disclosed at UniProt accession no: P02774-1, P02774-2, or P02774-3 (entry version 201) or a fragment thereof;
a Cystatin-C biomarker comprises an amino acid sequence having at least 70%, 80%, 90%, 95%, 99% or 100% sequence identity with the full-length of the amino acid sequence disclosed at UniProt accession no: P01034-1 (entry version 213) or a fragment thereof;
a Hepsin biomarker comprises an amino acid sequence having at least 70%, 80%, 90%, 95%, 99% or 100% sequence identity with the full-length of the amino acid sequence disclosed at UniProt accession no: P05981-1 (entry version 183) or a fragment thereof;
a Lactate Dehydrogenase (LDH) biomarker comprises an amino acid sequence having at least 70%, 80%, 90%, 95%, 99% or 100% sequence identity with the full-length of the amino acid sequence disclosed at UniProt accession no: P00338-1, P00338-2, P00338-3, P00338-4, or P00338-5 (entry version 224) or a fragment thereof; and/or
a Lactate Dehydrogenase (LDH) biomarker comprises an amino acid sequence having at least 70%, 80%, 90%, 95%, 99% or 100% sequence identity with the full-length of the amino acid sequence disclosed at UniProt accession no: P07195-1 (entry version 218) or a fragment thereof.

The biomarkers described herein may also be defined as a nucleic acid (either DNA or RNA) having at least 70%, 80%, 90%, 95%, 99% or 100% sequence identity with the full-length nucleic acid sequence encoding any of the above amino acid sequences.

Identity may be as defined using sequence comparisons made using FASTA and FASTP (see Pearson & Lipman, 1988. Methods in Enzymology 183: 63-98). Parameters are preferably set, using the default matrix, as follows: Gapopen (penalty for the first residue in a gap): −12 for proteins/-16 for DNA; Gapext (penalty for additional residues in a gap): −2 for proteins/-4 for DNA; KTUP word length: 2 for proteins/6 for DNA.

Pre-Treatment Predictive Biomarkers and Methods

By analysing further patient data obtained from clinical trial BGBC008 (NCT03184571) the authors have discovered that, prior to treatment of subjects having NSCLC, certain of the biomarkers described herein are present in the serum of “responder” subjects at higher levels than in the serum of “non-responder” subjects—see Examples 4, 5, 6, and 7. Similarly, prior to treatment of the subject certain of the biomarkers described herein are present in the serum of “responder” subjects at lower levels than in the serum of “non-responder” subjects. Thus, by assessing the activity, expression, or amount of one or more of the biomarkers described herein prior to treatment with an agent capable of inhibiting or reversing EMT or a chemotherapeutic agent it is possible to determine the likelihood of that subject being susceptible or responsive to treatment with said agent.

Accordingly, in a first aspect of this facet, the disclosure provides a method of predicting a cancer-related outcome in a subject having, suspected of having, or diagnosed with non-small cell lung cancer (NSCLC), the method comprising assessing the activity, expression, or amount of one or more of the biomarkers described herein in the subject, or in a sample derived from the subject.

In some embodiments the one or more biomarker is selected from the group consisting of: NT proBNP, Endostatin, IGFBP-2, VEGF-D, GDF-15, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl.

In some embodiments the one or more biomarker is selected from the group consisting of: NT proBNP, Endostatin, IGFBP-2, VEGF-D, LDH, and/or Axl.

In some embodiments the one or more biomarker is selected from the group consisting of: NT proBNP, Endostatin, IGFBP-2, and/or VEGF-D.

In some embodiments the one or more biomarker is selected from the group consisting of: AB-40, RAGE, VDBP, LDH, and/or Axl.

In some embodiments the one or more biomarker is selected from the group consisting of: AB-40, RAGE, and/or VDBP.

A “cancer-related outcome” as described herein is a clinical prediction or prognosis associated with cancer. In some embodiments the cancer-related outcome is susceptibility to treatment with an agent capable of inhibiting or reversing EMT or a chemotherapeutic agent.

In some embodiments the method comprises assessing the activity, expression, or amount of one or more of the biomarkers described herein in the subject, or in a sample derived from the subject, to obtain a sample profile of the one or more biomarker; and, making a prediction based on the sample profile of the one or more biomarker.

A “sample profile” as used herein is a profile of numerical values or numerical ranges corresponding to the activity, expression, or amount of respective biomarkers determined in the subject or sample derived from the subject.

In some embodiments of the method the prediction is made by comparing the sample profile to a control profile. A “control profile” as used herein is a profile of numerical values or numerical ranges corresponding to the activity, expression, or amount of the same biomarkers as for the sample profile, determined in a control subject or population of control subjects. Suitable control profiles are described in more detail below.

Prior to treatment of a subject, certain of the biomarkers described herein have been found to exhibit up-regulated expression in “responder” subjects—that is, certain of the biomarkers described herein have been found to be present in the serum of “responder” subjects at higher levels than in the serum of “non-responder” subjects. Similarly, prior to treatment of a subject, certain of the biomarkers described herein have been found to exhibit down-regulated expression in “responder” subjects—that is, certain of the biomarkers described herein have been found to be present in the serum of “responder” subjects at lower levels than in the serum of “non-responder” subjects.

This difference in expression level between “responder” and “non-responder” subjects can also be expressed in the opposite way. That is to say, prior to treatment of a subject, certain of the biomarkers described herein have been found to exhibit down-regulated expression in “non-responder” subjects—that is, certain of the biomarkers described herein have been found to be present in the serum of “non-responder” subjects at lower levels than in the serum of “responder” subjects. Similarly, prior to treatment of a subject, certain of the biomarkers described herein have been found to exhibit up-regulated expression in “non-responder” subjects—that is, certain of the biomarkers described herein have been found to be present in the serum of “non-responder” subjects at higher levels than in the serum of “responder” subjects.

The discovery of these differential patterns of expression mean that, by assessing the activity, expression, or amount of one or more of the biomarkers described herein prior to treatment of a subject with an agent, it is possible to determine the likelihood of that subject being susceptible or responsive to treatment with said agent. Accordingly, in particularly preferred embodiments the sample profile is obtained before the subject is contacted with or administered the agent capable of inhibiting or reversing EMT or a chemotherapeutic agent.

A subject who is “susceptible to treatment” with an agent capable of inhibiting or reversing EMT or a chemotherapeutic agent is one who is likely to, or who does, respond to treatment (or benefit from treatment) with an agent capable of inhibiting or reversing EMT or a chemotherapeutic agent. That is, a subject who is “susceptible to treatment” with an agent is a “responder” subject for that agent, as defined above. A prediction that is “indicative of susceptibility” is one which suggests or points towards the likelihood that a subject will respond to or benefit from treatment.

In some embodiments the predictive methods of the disclosure comprise assessing the activity, expression, or amount of one or more of the biomarkers described herein in the subject, or in a sample derived from the subject, to obtain a sample profile of the one or more biomarker; and, making a prediction based on the sample profile of the one or more biomarker. In some embodiments of the method the prediction is made by comparing the sample profile to a control profile, wherein the control profile is a profile of numerical values or numerical ranges corresponding to the activity, expression, or amount of the same biomarkers as for the sample profile, determined in a control subject or population of control subjects.

In some embodiments the control profile may be obtained from a population of control subjects having NSCLC. In some embodiments the control profile may be obtained from a population of control subjects not having NSCLC. In other embodiments the control profile may be obtained from a control subject or population of control subjects having NSCLC and previously found to lack susceptibility to treatment with an agent capable of inhibiting or reversing EMT or a chemotherapeutic agent (that is, non-responder subjects).

In some embodiments the control profile may a predetermined profile of biomarker expression, activity, or amount values, for example a profile of “mean” values, “threshold” values, or “standard ranges” of values. This predetermined profile of biomarker expression, activity, or amount values can be obtained, for example, from a control subject or population of control subjects having NSCLC, a control subject or population of control subjects not having NSCLC, or a control subject or population of control subjects having NSCLC and previously found to lack susceptibility to treatment with an agent capable of inhibiting or reversing EMT or a chemotherapeutic agent (that is, non-responder subjects).

In some other embodiments the control profile may be obtained from a control subject or population of control subjects having NSCLC and previously found to be susceptible to treatment with an agent capable of inhibiting or reversing EMT or a chemotherapeutic agent (that is, responder subjects). In some embodiments the control profile may a predetermined profile of biomarker expression, activity, or amount values, for example a profile of “threshold”, values or “standard ranges” of values. This predetermined profile of biomarker expression, activity, or amount values can be obtained, for example, from a control subject or population of control subjects having NSCLC and previously found to be susceptible to treatment with an agent capable of inhibiting or reversing EMT or a chemotherapeutic agent (that is, responder subjects).

In some other embodiments the control profile may be obtained from a control sample having a known amount (for example a “threshold” amount) of the same biomarkers as will be determined in the sample profile. This threshold amount can be obtained, for example, from non-responder or responder subjects as outlined above.

The skilled person is readily able to determine suitable “mean”, “threshold”, or “standard range” values for a particular biomarker.

For example, a “mean” value may be determined by: assessing the activity, expression, or amount of a particular biomarker in a population of subjects having (or not having) NSCLC; and, averaging the biomarker activity, expression, or amount determined to determine a “mean” value.

For example, a “mean” value may be determined by: assessing the activity, expression, or amount of a particular biomarker in a population of subjects; determining which subjects in the population are susceptible to treatment with an agent capable of inhibiting or reversing EMT or a chemotherapeutic agent; and, averaging the biomarker activity, expression, or amount determined in subjects found to be susceptible to treatment with an agent capable of inhibiting or reversing EMT or a chemotherapeutic agent (that is, responder subjects) to determine a “mean” value.

For example, a “threshold” value may be determined by: assessing the activity, expression, or amount of a particular biomarker in a population of subjects; determining which subjects in the population are susceptible to treatment with an agent capable of inhibiting or reversing EMT or a chemotherapeutic agent; and, selecting as appropriate the highest or lowest biomarker activity, expression, or amount determined in a subject found to be susceptible to treatment with an agent capable of inhibiting or reversing EMT or a chemotherapeutic agent (that is, a responder subject) as the “threshold value”.

For example, a “standard range” of values may be determined by: assessing the activity, expression, or amount of a particular biomarker in a population of subjects having (or not having) NSCLC; and, determining a “standard range” of values based on this assessment.

For example, a “standard range” of values may be determined by: assessing the activity, expression, or amount of a particular biomarker in a population of subjects; determining which subjects in the population are susceptible to treatment with an agent capable of inhibiting or reversing EMT or a chemotherapeutic agent; and, determining a “standard range” of values for subjects found to be susceptible to treatment with an agent capable of inhibiting or reversing EMT or a chemotherapeutic agent (that is, a responder subject) based on this assessment.

Where the control profile is a predetermined profile of biomarker expression, activity, or amount values, this may be provided as a “look-up table” or data record. Predetermined control profiles of this type may be a profile of numerical values or numerical ranges corresponding to a measure of central tendency (such as an average, median or mean) amount of the respective biomarkers determined in a plurality of control subjects (for example non-responder or responder subjects as outlined above). The skilled person is readily able to determine both a measure of central tendency and a measure of variability within a population using standard protein quantification techniques and suitable statistical tools.

Accordingly, in embodiments in which the control profile is: obtained from a population of control subjects having NSCLC; obtained from a population of control subjects not having NSCLC; obtained from a control subject or population of control subjects having NSCLC and previously found to lack susceptibility to treatment with an agent capable of inhibiting or reversing EMT or a chemotherapeutic agent (that is non-responder subjects); a predetermined profile of biomarker expression, activity, or amount values (for example a profile of “average, median, or mean” values or “standard ranges” of values) obtained from non-responder subjects; obtained from a control sample having a known “average, median, or mean” value of biomarkers indicative of non-responder subjects; a predetermined profile of biomarker expression, activity, or amount “threshold” values obtained from responder subjects; or, obtained from a control sample having a known “threshold” value of biomarkers indicative of responder subjects; then, a higher activity, expression, or amount of one or more of the biomarkers described herein in a sample profile as compared with the control profile may be indicative of susceptibility to treatment with an agent capable of inhibiting or reversing EMT or a chemotherapeutic agent. Similarly, a lower activity, expression, or amount of one or more of the biomarkers described herein in the sample profile as compared with a control profile may be indicative of susceptibility to treatment with an agent capable of inhibiting or reversing EMT or a chemotherapeutic agent.

In some such embodiments, the method may comprise assessing the activity, expression, or amount of one or more of biomarker selected from: MCP-3, SOD-1, BMP-9, IL-8, MIP-1 beta, MPO, VDBP and/or Axl; wherein a higher activity, expression, or amount of one or more of said biomarkers in the sample profile as compared with the control profile is indicative of susceptibility to treatment with an agent capable of inhibiting or reversing EMT or a chemotherapeutic agent.

In some such embodiments, the method may comprise assessing the activity, expression, or amount of one or more of biomarker selected from: NT proBNP, Endostatin, IGFBP-2, VEGF-D, GDF-15, AB-40, ALP, RAGE, CgA, HE4, ADM, B2M, Cystatin-C, Hepsin, and/or LDH; wherein a lower activity, expression, or amount of one or more of said biomarkers in the sample profile as compared with the control profile is indicative of susceptibility to treatment with an agent capable of inhibiting or reversing EMT or a chemotherapeutic agent.

In some such embodiments: and/or a lower activity, expression, or amount of NT proBNP; and/or a lower activity, expression, or amount of Endostatin; and/or a lower activity, expression, or amount of IGFBP-2; and/or a lower activity, expression, or amount of VEGF-D; and/or a lower activity, expression, or amount of GDF-15; and/or a lower activity, expression, or amount of AB-40; and/or a lower activity, expression, or amount of ALP; and/or a lower activity, expression, or amount of RAGE; and/or a lower activity, expression, or amount of CgA; and/or a lower activity, expression, or amount of HE4; and/or a lower activity, expression, or amount of ADM; and/or a lower activity, expression, or amount of B2M; and/or a lower activity, expression, or amount of Cystatin-C; and/or a lower activity, expression, or amount of Hepsin; and/or a lower activity, expression, or amount of LDH; and/or a higher activity, expression, or amount of MCP-3; and/or a higher activity, expression, or amount of SOD-1; and/or a higher activity, expression, or amount of BMP-9; and/or a higher activity, expression, or amount of IL-8; and/or a higher activity, expression, or amount of MIP-1 beta; and/or a higher activity, expression, or amount of MPO; and/or a higher activity, expression, or amount of VDBP; and/or a higher activity, expression, or amount of Axl; in the sample profile as compared with the control profile is indicative of susceptibility to treatment with an agent capable of inhibiting or reversing EMT or a chemotherapeutic agent.

In embodiments in which the control profile is: obtained from a control subject or population of control subjects having NSCLC and previously found to be susceptible to treatment with an agent capable of inhibiting or reversing EMT or a chemotherapeutic agent (that is, responder subjects); then, if the expression, activity, or amount values in the sample profile are above a “threshold” value typical of a responder subject (that is, a subject having NSCLC and previously found to be susceptible to treatment with an agent capable of inhibiting or reversing EMT or a chemotherapeutic agent) this is indicative of susceptibility to treatment with an agent capable of inhibiting or reversing EMT or a chemotherapeutic agent. Similarly, if the expression, activity, or amount values in the sample profile are below the “threshold” value typical of a responder subject (that is, a subject having NSCLC and previously found to be susceptible to treatment with an agent capable of inhibiting or reversing EMT or a chemotherapeutic agent) this is indicative of a lack of susceptibility to treatment with an agent capable of inhibiting or reversing EMT or a chemotherapeutic agent.

In embodiments in which the control profile is: a predetermined profile of “standard ranges” of biomarker expression, activity, or amount values obtained from responder subjects; then, if the expression, activity, or amount values in the sample profile are within the “standard range” of values typical of a responder subject (that is, a subject having NSCLC and previously found to be susceptible to treatment with an agent capable of inhibiting or reversing EMT or a chemotherapeutic agent) this is indicative of susceptibility to treatment with an agent capable of inhibiting or reversing EMT or a chemotherapeutic agent. Similarly, if the expression, activity, or amount values in the sample profile are outside the “standard range” of values typical of a responder subject (that is, a subject having NSCLC and previously found to be susceptible to treatment with an agent capable of inhibiting or reversing EMT or a chemotherapeutic agent) this is indicative of a lack of susceptibility to treatment with an agent capable of inhibiting or reversing EMT or a chemotherapeutic agent.

In embodiments in which the control profile is: a predetermined profile of “standard ranges” of biomarker expression, activity, or amount values obtained from non-responder subjects; then, if the expression, activity, or amount values in the sample profile are within the “standard range” of values typical of a non-responder subject (that is, a subject having NSCLC and previously found to lack susceptibility to treatment with an agent capable of inhibiting or reversing EMT or a chemotherapeutic agent) this is indicative of a lack of susceptibility to treatment with an agent capable of inhibiting or reversing EMT or a chemotherapeutic agent. Similarly, if the expression, activity, or amount values in the sample profile are outside the “standard range” of values typical of a non-responder subject (that is, a subject having NSCLC and previously found to lack susceptibility to treatment with an agent capable of inhibiting or reversing EMT or a chemotherapeutic agent) this may be indicative of susceptibility to treatment with an agent capable of inhibiting or reversing EMT or a chemotherapeutic agent.

In embodiments in which the control profile is: obtained from a population of control subjects not having NSCLC; then, if the expression, activity, or amount values in the sample profile are outside a “standard range” of values typical of subjects not having NSCLC, this is indicative of susceptibility to treatment with an agent capable of inhibiting or reversing EMT or a chemotherapeutic agent. Similarly, if the expression, activity, or amount values in the sample profile are within a “standard range” of values typical of subjects not having NSCLC, this is indicative of a lack of susceptibility to treatment with an agent capable of inhibiting or reversing EMT or a chemotherapeutic agent. In such cases, an expression, activity or amount value may be outside the “standard range” of values when it is either above the upper limit of normal or below the lower limit of normal for a given biomarker. The upper limit of normal and lower limit of normal are the limits of the normal distribution of a given biomarker in a population of control subjects not having NSCLC.

In embodiments in which the one or more biomarker comprises LDH, an expression or amount of LDH higher than 287 units per litre (U/L) may be indicative of susceptibility to treatment with an agent capable of inhibiting or reversing EMT or a chemotherapeutic agent.

The skilled person is readily able to determine whether the sample profile is indicative of susceptibility or lack of susceptibility to treatment with an agent capable of inhibiting or reversing EMT or a chemotherapeutic agent using suitable statistical tools and comparison with the appropriate control.

In some embodiments the sample profile may be compared with two or more control profiles as described above.

As used herein, the term “one or more” means 1, 2, 3, 4, 5, 6, 7, 8, 9, 10, 11, 12, 13, 14, 15, 16, 17, 18, 19, or 20 of the biomarkers described herein. In some embodiments the one or more biomarker is NT proBNP. In some embodiments the one or more biomarker is Endostatin. In some embodiments the one or more biomarker is IGFBP-2. In some embodiments the one or more biomarker is VEGF-D. In some embodiments the one or more biomarker is GDF-15. In some embodiments the one or more biomarker is AB-40. In some embodiments the one or more biomarker is ALP. In some embodiments the one or more biomarker is MCP-3. In some embodiments the one or more biomarker is SOD-1. In some embodiments the one or more biomarker is BMP-9. In some embodiments the one or more biomarker is IL-8. In some embodiments the one or more biomarker is MIP-1 beta. In some embodiments the one or more biomarker is RAGE. In some embodiments the one or more biomarker is MPO. In some embodiments the one or more biomarker is CgA. In some embodiments the one or more biomarker is HE4. In some embodiments the one or more biomarker is VDBP. In some embodiments the one or more biomarker is Cystatin-C. In some embodiments the one or more biomarker is Hepsin. In some embodiments the one or more biomarker is Axl. In some embodiments the one or more biomarker is LDH.

In some embodiments the method may comprise assessing the activity, expression, or amount of at least 2, 3, 4, 5, 6, 7, 8, 9, 10, 11, 12, 13, 14, 15, 20 of said biomarkers in the subject, or in a sample derived from the subject. In some preferred embodiments the method may comprise assessing the activity, expression, or amount of NT proBNP and at least 1, 2, 3, 4, 5, 6, 7, 8, 9, 10, 11, 12, 13, 14, 15, 20 further biomarkers described herein in the subject, or in a sample derived from the subject. In some preferred embodiments the method may comprise assessing the activity, expression, or amount of Endostatin and at least 1, 2, 3, 4, 5, 6, 7, 8, 9, 10, 11, 12, 13, 14, 15, 20 further biomarkers described herein in the subject, or in a sample derived from the subject. In some preferred embodiments the method may comprise assessing the activity, expression, or amount of IGFBP-2, and at least 1, 2, 3, 4, 5, 6, 7, 8, 9, 10, 11, 12, 13, 14, 15, 20 further biomarkers described herein in the subject, or in a sample derived from the subject. In some preferred embodiments the method may comprise assessing the activity, expression, or amount of VEGF-D, and at least 1, 2, 3, 4, 5, 6, 7, 8, 9, 10, 11, 12, 13, 14, 15, 20 further biomarkers described herein in the subject, or in a sample derived from the subject. In some preferred embodiments the method may comprise assessing the activity, expression, or amount of LDH, and at least 1, 2, 3, 4, 5, 6, 7, 8, 9, 10, 11, 12, 13, 14, 15, 20 further biomarkers described herein in the subject, or in a sample derived from the subject. In some preferred embodiments the method may comprise assessing the activity, expression, or amount of Axl, and at least 1, 2, 3, 4, 5, 6, 7, 8, 9, 10, 11, 12, 13, 14, 15, 20 further biomarkers described herein in the subject, or in a sample derived from the subject.

In some preferred embodiments the method may comprise assessing the activity, expression, or amount of NT proBNP, Endostatin, and at least 1, 2, 3, 4, 5, 6, 7, 8, 9, 10, 11, 12, 13, 14, 15, 20 further biomarkers described herein in the subject, or in a sample derived from the subject. In other preferred embodiments the method may comprise assessing the activity, expression, or amount of NT proBNP, IGFBP-2, and at least 1, 2, 3, 4, 5, 6, 7, 8, 9, 10, 11, 12, 13, 14, 15, 20 further biomarkers described herein in the subject, or in a sample derived from the subject. In some preferred embodiments the method may comprise assessing the activity, expression, or amount of NT proBNP, VEGF-D, and at least 1, 2, 3, 4, 5, 6, 7, 8, 9, 10, 11, 12, 13, 14, 15, 20 further biomarkers described herein in the subject, or in a sample derived from the subject. In some preferred embodiments the method may comprise assessing the activity, expression, or amount of Endostatin, IGFBP-2, and at least 1, 2, 3, 4, 5, 6, 7, 8, 9, 10, 11, 12, 13, 14, 15, 20 further biomarkers described herein in the subject, or in a sample derived from the subject. In some preferred embodiments the method may comprise assessing the activity, expression, or amount of AB-40, RAGE, VDBP, and at least 1, 2, 3, 4, 5, 6, 7, 8, 9, 10, 11, 12, 13, 14, 15, 20 further biomarkers described herein in the subject, or in a sample derived from the subject.

Subject

A “subject” as referred to herein is any species of animal classified as a mammal, and includes but is not restricted to, domestic and farm animals, primates and humans. In some preferred embodiments the subject is a human of any sex or race. In some embodiments, the human is an adult human.

In some embodiments the predictive methods described herein are performed before the subject receives a treatment or course of treatment for NSCLC. In other embodiments the predictive method is performed during or after a treatment or course of treatment for NSCLC. In some embodiments the treatment or course of treatment for NSCLC is an agent capable of inhibiting or reversing EMT. In other embodiments the treatment or course of treatment for NSCLC is a chemotherapeutic treatment that is not an agent capable of inhibiting or reversing EMT.

In some embodiments of the predictive methods described herein the subject has previously been treated with an agent capable of inhibiting or reversing EMT or a chemotherapeutic agent. In some embodiments the subject has previously been treated with an agent selected from: a PHGDH inhibitor, a Slfn11 inhibitor, an Axl inhibitor, or an Akt3 inhibitor.

In some embodiments of the predictive methods described herein the subject has previously been treated with an agent that is different to the agent capable of inhibiting or reversing EMT or chemotherapeutic agent for which the subject's response is being predicted. That is, the subject has previously been treated with an agent that is not the agent which the predictive method is determining the subject's susceptibility to. Accordingly, in some embodiments, the subject has not previously been treated with an agent capable of inhibiting or reversing EMT or a chemotherapeutic agent. In some preferred embodiments, the subject has not previously been treated with an Axl inhibitor. In other preferred embodiments, the subject has not previously been treated with an Akt3 inhibitor.

Post-Treatment Changes in Biomarkers

In some embodiments of this aspect of the disclosure, the predictive method further comprises a subsequent step of assessing the activity, expression, or amount of one or more biomarker in the subject, or in a sample derived from the subject, to obtain a second sample profile of one or more of the biomarkers described herein after the subject has received a treatment or course of treatment an agent capable of inhibiting or reversing EMT or a chemotherapeutic agent.

That is, in some embodiments, the predictive method comprises:
(a) determining if a subject is susceptible to treatment with an agent capable of inhibiting or reversing EMT or a chemotherapeutic agent by assessing the activity, expression, or amount of one or more biomarker in the subject, as outlined above;
(b) administering to the subject a therapeutically effective amount of an agent capable of inhibiting or reversing EMT or a chemotherapeutic agent; and
(c) subsequently assessing the activity, expression, or amount of one or more biomarker in the subject, or in a sample derived from the subject.

In some embodiments, the predictive method comprises:
(a) determining if a subject is susceptible to treatment with an agent capable of inhibiting or reversing EMT or a chemotherapeutic agent by assessing the activity, expression, or amount of one or more biomarker in the subject, or in a sample derived from the subject, wherein the one or more biomarker is selected from the group consisting of NT proBNP, Endostatin, IGFBP-2, VEGF-D, GDF-15, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl;
(b) administering to the subject a therapeutically effective amount of an agent capable of inhibiting or reversing EMT or a chemotherapeutic agent; and
(c) subsequently assessing the activity, expression, or amount of one or more biomarker in the subject, or in a sample derived from the subject.

In some preferred embodiments the methods comprise subsequently assessing the activity, expression, or amount of Axl in the subject, or in a sample derived from the subject. In some embodiments an increase in the activity, expression, or amount of Axl in the second sample profile as compared with a control profile after contacting the subject with a cancer therapeutic agent is indicative of higher susceptibility to treatment with an agent capable of inhibiting or reversing EMT or a chemotherapeutic agent. In some preferred embodiments the control profile is obtained from the same subject prior to treatment with an agent capable of inhibiting or reversing EMT or a chemotherapeutic agent.

Post-Treatment Predictive Biomarkers and Methods

By analysing further patient data obtained from clinical trial BGBC008 (NCT03184571) the authors have discovered that, following treatment of subjects having NSCLC with an agent capable of inhibiting or reversing EMT or a chemotherapeutic agent, the levels of certain of the biomarkers described herein increases in the serum of “responder” subjects but not in the serum of “non-responder” subjects. Similarly, following treatment with an agent capable of inhibiting or reversing EMT or a chemotherapeutic agent, the levels of certain of the biomarkers described herein decreases in the serum of “responder” subjects but not in the serum of “non-responder” subjects. Thus, by assessing the activity, expression, or amount of one or more of the biomarkers described herein prior to and following treatment with an agent capable of inhibiting or reversing EMT or a chemotherapeutic agent it is possible to determine or confirm that the subject is susceptible or responsive to treatment with said agent.

Accordingly, in a second aspect of this facet the disclosure provides a method of predicting a cancer-related outcome in a subject having, suspected of having, or diagnosed with non-small cell lung cancer (NSCLC), the method comprising assessing the activity, expression, or amount of one or more of the biomarkers described herein in a subject, or in a sample derived from the subject after the subject has been contacted with an agent capable of inhibiting or reversing EMT or a chemotherapeutic agent. In some embodiments the cancer-related outcome is susceptibility to treatment with an agent capable of inhibiting or reversing EMT or a chemotherapeutic agent.

In some embodiments the method comprises assessing the activity, expression, or amount of one or more of the biomarkers described herein in the subject, or in a sample derived from the subject, to obtain a sample profile of the one or more biomarker; and, making a prediction based on the sample profile of the one or more biomarker. In some embodiments of the method the prediction is made by comparing the sample profile to a control profile. In preferred embodiments the sample profile is obtained after contacting the subject with an agent capable of inhibiting or reversing EMT or a chemotherapeutic agent.

A “control profile” as used herein is a profile of numerical values or numerical ranges corresponding to the activity, expression, or amount of the same biomarkers as for the sample profile, determined in a control subject or population of control subjects.

In preferred embodiments of this aspect of the disclosure the control profile is obtained from the same subject prior to treatment with an agent capable of inhibiting or reversing EMT or a chemotherapeutic agent. In some embodiments the control profile is obtained from a population of control subjects having NSCLC. In other embodiments, the control profile is a predetermined profile of biomarker expression, activity, or amount values, for example a profile of “threshold” values or “standard ranges” of values.

Accordingly, in embodiments in which the control profile is obtained from the same subject prior to treatment with an agent capable of inhibiting or reversing EMT or a chemotherapeutic agent, an increase in the activity, expression, or amount of one or more biomarker in the sample profile as compared with a control profile after contacting the subject with an agent capable of inhibiting or reversing EMT or a cancer therapeutic agent may be indicative of susceptibility to treatment with an agent capable of inhibiting or reversing EMT or a chemotherapeutic agent. In such embodiments, a decrease in the activity, expression, or amount of one or more biomarker in the sample profile as compared with a control profile after contacting the subject with a cancer therapeutic agent may be indicative of higher susceptibility to treatment with an agent capable of inhibiting or reversing EMT or a chemotherapeutic agent.

In some such embodiments, an increase in the activity, expression, or amount of one or more biomarker in the sample profile as compared with a control profile after contacting the subject with a cancer therapeutic agent may be indicative of lower susceptibility to treatment with an agent capable of inhibiting or reversing EMT or a chemotherapeutic agent. In some such embodiments, a decrease in the activity, expression, or amount of one or more biomarker in the sample profile as compared with a control profile after contacting the subject with a cancer therapeutic agent may be indicative of lower susceptibility to treatment with an agent capable of inhibiting or reversing EMT or a chemotherapeutic agent.

In some preferred embodiments the one or more biomarker includes at least Axl. In some embodiments the one or more biomarker is Axl. In some embodiments an increase in the activity, expression, or amount of Axl in the sample profile as compared with a control profile after contacting the subject with a cancer therapeutic agent is indicative of higher susceptibility to treatment with an agent capable of inhibiting or reversing EMT or a chemotherapeutic agent.

Subject

A “subject” as referred to herein is any species of animal classified as a mammal, and includes but is not restricted to, domestic and farm animals, primates and humans. In some preferred embodiments the subject is a human of any sex or race. In some embodiments, the human is an adult human.

In some embodiments of this aspect of the disclosure, the subject was selected for treatment with the agent capable of inhibiting or reversing EMT or a chemotherapeutic agent, using a predictive method according to the first aspect of this facet of the disclosure outlined above.

Agents Capable of Inhibiting or Reversing EMT

The present disclosure provides methods for predicting a cancer-related outcome in a subject having, suspected of having, or diagnosed with non-small cell lung cancer (NSCLC). A “cancer-related outcome” is a clinical prediction or prognosis associated with cancer.

The agents capable of inhibiting or reversing EMT for use in this NSCLC facet of the disclosure are defined as described above in the AML facet (see identically titled section above on page 14, line 19 to page 17, line 2).

In some preferred embodiments the further cancer treatment is a PD-1 inhibitor or PD-L1 inhibitor. In some embodiments the further cancer treatment is a monoclonal antibody such as pembrolizumab, alemtuzumab, bevacizumab, cetuximab, ibritumomab, tiuxetan, or rituximab. In some preferred embodiments the further cancer treatment is a microtubule polymer stabilizer such as the taxanes paclitaxel and docetaxel. In some preferred embodiments the further cancer treatment is a tyrosine kinase inhibitors such as erlotinib hydrochloride, gefitinib, imatinib mesylate, and semaxanib. In some particularly preferred embodiments the further cancer treatment is pembrolizumab. In other particularly preferred embodiments the further cancer treatment is docetaxel. In yet other particularly preferred embodiments the further cancer treatment is erlotinib.

Assessing Biomarker Activity, Expression, Amount

In some embodiments of the predictive methods described herein, assessing the activity, expression, or amount of one or more biomarker in a subject, or in a sample derived from a subject, comprises: contacting a sample, or an extract from a sample, with at least 1, 2, 3, 4, 5, 6, 7, 8, 9, 10 or more reagents for detecting the activity, expression, or amount of one or more biomarkers, each of which is selective for a particular biomarker; and, detecting and/or quantifying said reagents for detecting.

In some embodiments comprising assessing the activity of LDH, assessing the activity of LDH comprises determining the enzymatic activity of LDH in the subject or sample derived from the subject. Preferably the enzymatic activity is assessed by determining the level or amount of a substrate or product of the enzymatic reaction catalysed by LDH in the subject or sample derived from the subject. In particularly preferred embodiments the enzymatic activity is assessed by determining the level or amount of reduced nicotinamide adenine dinucleotide (NADH) in the subject or sample derived from the subject. In some embodiments this may be determined using a colorimetric assay, for example, the colorimetric assay available from Sigma-Aldrich with catalogue number MAK066. Other suitable assays for assessing activity of LDH are well known to the skilled person. In particularly preferred embodiments the activity of LDH is assessed by determining the enzymatic activity of LDH in blood or serum.

In some preferred embodiments, assessing the expression or amount of the one or more biomarker comprises determining the level of the one or more biomarker protein or mRNA in the subject or sample derived from the subject. Preferably the expression or amount of the one or more biomarker is assessed by determining the level of protein expression. In particularly preferred embodiments the expression or amount of the one or more biomarker is assessed by determining the level of protein expression in serum.

The term “expression” refers to the transcription of a gene's DNA template to produce the corresponding mRNA and translation of this mRNA to produce the corresponding gene product (i.e., a peptide, polypeptide, or protein) as well as the “expression” of a protein in one or more forms that may have been modified post translation.

In some embodiments determining the level of the one or more biomarker protein or mRNA comprises: contacting a sample, or an extract from a sample, with at least 1, 2, 3, 4, 5, 6, 7, 8, 9, 10 or more specific binding members, each of which selectively binds to a respective biomarker protein or mRNA; and, detecting and/or quantifying formation of complex formed by said specific binding member and said biomarker protein or mRNA. In some embodiments the specific binding member may comprise an antibody molecule or a binding fragment thereof.

A range of suitable techniques for determining protein and mRNA expression levels are well known in the art, for example microarray analysis, Western blotting, and PCR techniques such as QPCR. In some embodiments, determining the expression or amount of the one or more biomarker protein or mRNA may comprise use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.

In some embodiments, the expression or amount of the one or more biomarker is determined as an absolute amount of the respective biomarker protein or mRNA in the sample. In such cases, the respective biomarker protein or mRNA may be quantified in the sample and the amount expressed as a measure of the sample, for example the volume, mass, or total protein content of the sample.

In some embodiments, the expression or amount of the one or more biomarker is determined as a relative amount of the respective biomarker protein or mRNA in the sample, wherein the relative amount is determined relative to a reference protein or mRNA in the sample. Determining the relative amount of biomarker protein or mRNA in this way allows for normalisation to, e.g., account for differences in total protein concentration and to remove bias from sample to sample.

Preferably the reference protein or mRNA is one whose expression or amount does not vary significantly between subjects having cancer and subjects not having cancer, in particular NSCLC. That is, the reference protein or mRNA is preferably one whose expression or amount is not altered by NSCLC. Accordingly, the reference protein or mRNA may comprise one or more protein or mRNA which is not one of the biomarkers described herein. When expressing the expression or amount of the one or more biomarker as a relative amount, the absolute amount of the respective biomarker protein or mRNA in the sample may be divided by the absolute amount of the reference protein or mRNA in the sample.

Assessing Biomarker Expression at the RNA Level

Gene expression can be detected at the RNA level. RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland). Typical assay formats utilising ribonucleic acid hybridisation include nuclear run-on assays, RT-PCR, RNase protection assays (Melton et al., Nuc. Acids Res. 12:7035), Northern blotting and In Situ hybridization. Gene expression can also be detected by microarray analysis as described below.

Assessing Biomarker Expression at the Protein Level

Preferably biomarker level is assessed by measuring protein expression. Altered gene or protein expression may also be detected by measuring the polypeptides encoded by the gene. This may be achieved by using molecules which bind to the polypeptides encoded by the biomarker gene. Suitable molecules/agents which bind either directly or indirectly to the polypeptides in order to detect the presence of the protein include naturally occurring molecules such as peptides and proteins, for example antibodies, or they may be synthetic molecules.

Antibodies may be derived from commercial sources or through techniques which are familiar to those skilled in the art. In one embodiment, and where altered expression manifests itself through the expression of alteration of post translationally-modified forms of a protein biomarker, antibodies specific for those different forms may be used. For the purposes of this disclosure, the term “antibody”, unless specified to the contrary, includes whole antibodies, or fragments of whole antibodies which retain their binding activity for a target antigen. Such fragments include Fv, F(ab′) and F(ab′)2 fragments, as well as single chain antibodies (scFv). Furthermore, the antibodies and fragments thereof may be humanised antibodies, for example as described in EP239400A. For example: monoclonal and polyclonal antibodies, recombinant antibodies, proteolytic and recombinant fragments of antibodies (Fab, Fv, scFv, diabodies), single-domain antibodies (VHH, sdAb, nanobodies, IgNAR, VNAR), and proteins unrelated to antibodies, which have been engineered to have antibody-like specific binding. Antibodies may be bound to a solid support and/or packaged into kits in a suitable container along with suitable reagents, controls, instructions and the like.

Arrays

Array technology and the various techniques and applications associated with it are well known in the art. Array technology overcomes the disadvantages with traditional methods in molecular biology, which generally work on a “one gene in one experiment” basis, resulting in low throughput and the inability to appreciate the “whole picture” of gene function. In the context of the products and methods of the present disclosure, array technology can be used, for example, in the analysis of the expression of biomarker proteins or mRNA.

In general, any library or group of samples may be arranged in an orderly manner into an array, by spatially separating the members of the library or group. Examples of suitable libraries for arraying include nucleic acid libraries (including DNA, cDNA, oligonucleotide, etc. libraries), peptide, polypeptide and protein libraries, as well as libraries comprising any molecules, such as ligand libraries, among others. Accordingly, where reference is made to a “library” in this document, unless the context dictates otherwise, such reference should be taken to include reference to a library in the form of an array.

Proteins, polypeptides, etc., may also be immobilised in arrays. For example, antibodies have been used in microarray analysis of the proteome using protein chips (Borrebaeck C A, 2000, Immunol Today 21(8):379-82). Polypeptide arrays are reviewed in, for example, MacBeath and Schreiber, 2000, Science, 289(5485):1760-1763.

Sample

Suitable samples include, but are not limited to, tissue samples such as tissue biopsy, blood, urine, buccal scrapes etc., as well as serum, plasma, or tissue culture supernatant samples. In preferred embodiments the expression or amount of the one or more biomarker is assessed by determining the level of protein expression in the sample. In some preferred embodiments the sample is a blood, serum, or plasma sample. In some particularly preferred embodiments the sample is a serum sample.

When assessing the expression or amount of the one or more biomarker proteins or mRNA in serum or plasma samples derived from a subject, samples are removed and subjected to analytical techniques such as flow cytometry, mass cytometry (CyTOF), ELISA, PET, and SELDI-TOF MS. In some embodiments, the method may comprise extracting RNA from said sample and detecting gene expression by QPCR. In some other embodiments, gene expression may be detected by detecting protein products by, for example, Western Blot.

In some embodiments, the level of expression of the one or more biomarker is assessed by determining the copy number of the gene encoding the one or more biomarker. Copy number (i.e. gene duplication events) may be determined using standard techniques known in the art, e.g. using a DNA chip as described in Jiang et al. (Jiang Q, Ho Y Y, Hao L, Nichols Berrios C, Chakravarti A. Copy number variants in candidate genes are genetic modifiers of Hirschsprung disease. PLoS One. 2011; 6(6)).

Is some preferred embodiments of the predictive methods described herein, the method is performed in vitro or ex vivo.

Methods of Selecting a Subject for Treatment

A third aspect of this facet of the present disclosure relates to methods of selecting subjects having, suspected of having, or diagnosed with non-small cell lung cancer (NSCLC) for treatment with an agent capable of inhibiting or reversing EMT or a chemotherapeutic agent. In some embodiments the method comprises: identifying subjects susceptible to treatment with an agent capable of inhibiting or reversing EMT or a chemotherapeutic agent using a predictive method defined in accordance with the above described first and second aspects of this facet of the disclosure; and, selecting thus identified subjects for treatment.

In some embodiments this aspect relates to methods of selecting subjects having, suspected of having, or diagnosed with non-small cell lung cancer (NSCLC) for continued treatment with an agent capable of inhibiting or reversing EMT or a chemotherapeutic agent. In some embodiments the method comprises: identifying subjects susceptible to treatment with an agent capable of inhibiting or reversing EMT or a chemotherapeutic agent using a predictive method defined in accordance with the above described second aspect of this facet of the disclosure; and, selecting thus identified subjects for treatment.

In some embodiments of this aspect of the disclosure, the treatment comprises administering to the subject a therapeutically effective amount of an agent capable of inhibiting or reversing EMT or a chemotherapeutic agent as described herein. Accordingly, in some embodiments the treatment comprises an Axl inhibitor, Akt3 inhibitor, Slfn11 inhibitor, or PHGDH inhibitor. In some preferred embodiments the treatment comprises an Axl inhibitor, for example, BGB324/R428/bemcentinib, cabozantinib, TP-0903, foretinib, merestinib, bosutinib, gilteritinib, crizotinib, amuvatinib, sunitinib, sitravatinib, LDC1267, or UNC2025. Other Axl inhibitors include the anti-Axl antibodies described in WO2015/193428, WO2015/193430, WO2016/097370, and WO2016/166296. In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In some other preferred embodiments, the treatment comprises an Akt3 inhibitor. In some embodiments the Akt3 inhibitor is an Akt3 inhibitor disclosed in WO2016/102672.

In some embodiments of this aspect of the disclosure, the treatment is administered as a single agent. In other embodiments, the treatment is administered in combination with a further cancer treatment. Suitable further cancer treatments are outlined in detail above.

In some preferred embodiments the further cancer treatment is a PD-1 inhibitor or PD-L1 inhibitor. In some embodiments the further cancer treatment is a monoclonal antibody such as pembrolizumab, alemtuzumab, bevacizumab, cetuximab, ibritumomab, tiuxetan, or rituximab. In some preferred embodiments the further cancer treatment is a microtubule polymer stabilizer such as the taxanes paclitaxel and docetaxel. In some preferred embodiments the further cancer treatment is a tyrosine kinase inhibitor such as erlotinib hydrochloride, gefitinib, imatinib mesylate, and semaxanib. In some particularly preferred embodiments the further cancer treatment is pembrolizumab. In other particularly preferred embodiments the further cancer treatment is docetaxel. In yet other particularly preferred embodiments the further cancer treatment is erlotinib.

Kits

A fourth aspect of this facet of the present disclosure relates to diagnostic kits and test devices. In some embodiments the disclosure provides a diagnostic kit comprising 1, 2, 3, 4, 5, 6, 7, 8, 9, 10 or more reagents for detecting the activity, expression, or amount of one or more of the biomarkers described herein, each of which is selective for a particular biomarker. In some embodiments each of the reagents for detecting is selective for a biomarker selected from the group consisting of: NT proBNP, Endostatin, IGFBP-2, VEGF-D, GDF-15, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl.

In some embodiments the diagnostic kit or test device comprises 1, 2, 3, 4, 5, 6, 7, 8, 9, 10 or more specific binding members, each of which selectively binds to one of the biomarkers described herein; and, one or more reagents for detecting said one or more specific binding members, or one or more reagents for detecting and/or quantifying formation of a complex formed by said specific binding member and said biomarker. In some embodiments each specific binding member selectively binds to a biomarker selected from the group consisting of: NT proBNP, Endostatin, IGFBP-2, VEGF-D, GDF-15, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl.

In some embodiments each reagent for detecting or specific binding member is selective for a biomarker selected from the group: NT proBNP, Endostatin, IGFBP-2, VEGF-D, GDF-15, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl.

In some embodiments the diagnostic kit or test device comprises a specific binding member which selectively binds to NT proBNP, and at least 1, 2, 3, 4, 5, 6, 7, 8, 9, 10, or more specific binding members, each of which selectively binds to a biomarker selected from the group consisting of: Endostatin, IGFBP-2, VEGF-D, GDF-15, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl.

In some embodiments the diagnostic kit or test device comprises a specific binding member which selectively binds to Endostatin, and at least 1, 2, 3, 4, 5, 6, 7, 8, 9, 10, or more specific binding members, each of which selectively binds to a biomarker selected from the group consisting of NT proBNP, IGFBP-2, VEGF-D, GDF-15, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl.

In some embodiments the diagnostic kit or test device comprises a specific binding member which selectively binds to IGFBP-2, and at least 1, 2, 3, 4, 5, 6, 7, 8, 9, 10, or more specific binding members, each of which selectively binds to a biomarker selected from the group consisting of: NT proBNP, Endostatin, VEGF-D, GDF-15, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl.

In some embodiments the diagnostic kit or test device comprises a specific binding member which selectively binds to VEGF-D, and at least 1, 2, 3, 4, 5, 6, 7, 8, 9, 10, or more specific binding members, each of which selectively binds to a biomarker selected from the group consisting of: NT proBNP, Endostatin, IGFBP-2, GDF-15, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl.

In some embodiments the diagnostic kit or test device comprises a specific binding member which selectively binds to GDF-15, and at least 1, 2, 3, 4, 5, 6, 7, 8, 9, 10, or more specific binding members, each of which selectively binds to a biomarker selected from the group consisting of: NT proBNP, Endostatin, IGFBP-2, VEGF-D, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl.

In some embodiments the diagnostic kit or test device comprises a specific binding member which selectively binds to AB-40, and at least 1, 2, 3, 4, 5, 6, 7, 8, 9, 10, or more specific binding members, each of which selectively binds to a biomarker selected from the group consisting of: NT proBNP, Endostatin, IGFBP-2, VEGF-D, GDF-15, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl.

In some embodiments the diagnostic kit or test device comprises a specific binding member which selectively binds to AB-40, a specific binding member which selectively binds to RAGE, and at least 1, 2, 3, 4, 5, 6, 7, 8, 9, 10, or more specific binding members, each of which selectively binds to a biomarker selected from the group consisting of: NT proBNP, Endostatin, IGFBP-2, VEGF-D, GDF-15, ALP, MCP-3, SOD-1, BMP-9, IL-8, MIP-1 beta, MPO, CgA, HE4, ADM, B2M, VDBP, Cystatin-C, Hepsin, LDH, and/or Axl.

In some embodiments the diagnostic kit or test device comprises a specific binding member which selectively binds to AB-40, a specific binding member which selectively binds to RAGE, a specific binding member which selectively binds to VDBP, and at least 1, 2, 3, 4, 5, 6, 7, 8, 9, 10, or more specific binding members, each of which selectively binds to a biomarker selected from the group consisting of: NT proBNP, Endostatin, IGFBP-2, VEGF-D, GDF-15, ALP, MCP-3, SOD-1, BMP-9, IL-8, MIP-1 beta, MPO, CgA, HE4, ADM, B2M, Cystatin-C, Hepsin, LDH, and/or Axl.

In some embodiments of this aspect of the disclosure the diagnostic kit or test device comprises a plurality of said specific binding members provided in the form of an array on a substrate or conjugated to a plurality of particles, such as beads or microspheres. The particles may be encoded with a detectable label. In some embodiments the specific binding member comprises an antibody molecule or a binding fragment thereof.

In some embodiments of the diagnostic kit or test device detecting formation of a complex formed by a specific binding member and a biomarker is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.

In some embodiments of the diagnostic kit or test device, the kit or test device comprises no more than 15, 20, 25, 30, 40, or 50 reagents for detecting. In some embodiments of the diagnostic kit or test device, the kit or test device comprises no more than 15, 20, 25, 30, 40, or 50 specific binding members.

The present disclosure also provides use of such diagnostic kits and test devices in methods of predicting a cancer-related outcome in a subject. In preferred embodiments the cancer-related outcome is susceptibility to treatment with an agent capable of inhibiting or reversing EMT or a chemotherapeutic agent as described herein.

In some embodiments the agent capable of inhibiting or reversing EMT or chemotherapeutic agent is an Axl inhibitor, Akt3 inhibitor, Slfn11 inhibitor, or PHGDH inhibitor. In some preferred embodiments the agent capable of inhibiting or reversing EMT or chemotherapeutic agent is an Axl inhibitor, for example BGB324/R428/bemcentinib, cabozantinib, TP-0903, foretinib, merestinib, bosutinib, gilteritinib, crizotinib, amuvatinib, sunitinib, sitravatinib, LDC1267, or UNC2025. Other Axl inhibitors include the anti-Axl antibodies described in WO2015/193428, WO2015/193430, WO2016/097370, and WO2016/166296. In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In some embodiments of the diagnostic kit or test device for use in a predictive method, the subject has, is suspected of having, or has been diagnosed with NSCLC.

In some embodiments the method may be as defined in accordance with the above described first and second aspects of this facet of the disclosure.

A fifth aspect of this facet of the present disclosure relates to the use of a reagent for detecting the activity, expression, or amount of a biomarker selected from the group consisting of: NT proBNP, Endostatin, IGFBP-2, VEGF-D, GDF-15, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl, in a method of predicting a cancer-related outcome in a subject.

A sixth aspect of this facet of the disclosure relates to the use of 1, 2, 3, 4, 5, 6, 7, 8, 9, 10 or more reagents for detecting the activity, expression, or amount a biomarker, each of which is selective for a particular biomarker selected from the group consisting of: NT proBNP, Endostatin, IGFBP-2, VEGF-D, GDF-15, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl, in a method of manufacture of a diagnostic kit or test device for use in a method of predicting a cancer-related outcome in a subject. In some embodiments of these uses the reagent for detecting comprises a specific binding member selective for a particular biomarker.

The specific binding member may comprise an antibody molecule or a binding fragment thereof.

In some embodiments the method may be as defined in accordance with the above described first and second aspects of this facet of the disclosure.

Uses

A seventh aspect of this facet of the present disclosure relates to the use of one or more of: NT proBNP, Endostatin, IGFBP-2, VEGF-D, GDF-15, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl, as a biomarker for determining if a subject is susceptible to treatment with an agent capable of inhibiting or reversing EMT or a chemotherapeutic agent.

In some embodiments the use relates to use of NT proBNP and at least 1, 2, 3, 4, 5, 6, 7, 8, 9, 10, or more of: Endostatin, IGFBP-2, VEGF-D, GDF-15, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl, as a biomarker for determining if a subject is susceptible to treatment with an agent capable of inhibiting or reversing EMT or a chemotherapeutic agent. In some embodiments the use relates to use of Endostatin, and at least 1, 2, 3, 4, 5, 6, 7, 8, 9, 10, or more of: NT proBNP, IGFBP-2, VEGF-D, GDF-15, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl, as a biomarker for determining if a subject is susceptible to treatment with an agent capable of inhibiting or reversing EMT or a chemotherapeutic agent.

In some embodiments the use relates to use of IGFBP-2, and at least 1, 2, 3, 4, 5, 6, 7, 8, 9, 10, or more of: NT proBNP, Endostatin, VEGF-D, GDF-15, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl, as a biomarker for determining if a subject is susceptible to treatment with an agent capable of inhibiting or reversing EMT or a chemotherapeutic agent.

In some embodiments the use relates to use of VEGF-D, and at least 1, 2, 3, 4, 5, 6, 7, 8, 9, 10, or more of: NT proBNP, IGFBP-2, Endostatin, GDF-15, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl, as a biomarker for determining if a subject is susceptible to treatment with an agent capable of inhibiting or reversing EMT or a chemotherapeutic agent.

In some embodiments the use relates to use of GDF-15, and at least 1, 2, 3, 4, 5, 6, 7, 8, 9, 10, or more of: NT proBNP, IGFBP-2, Endostatin, VEGF-D, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl, as a biomarker for determining if a subject is susceptible to treatment with an agent capable of inhibiting or reversing EMT or a chemotherapeutic agent.

In some embodiments the use relates to use of AB-40, and at least 1, 2, 3, 4, 5, 6, 7, 8, 9, 10, or more of: NT proBNP, IGFBP-2, Endostatin, VEGF-D, GDF-15, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl, as a biomarker for determining if a subject is susceptible to treatment with an agent capable of inhibiting or reversing EMT or a chemotherapeutic agent.

In some preferred embodiments the one or more biomarker is NT proBNP. In some preferred embodiments the one or more biomarker is Endostatin. In some preferred embodiments the one or more biomarker is IGFBP-2. In some preferred embodiments the one or more biomarker is VEGF-D. In some preferred embodiments the one or more biomarker is GDF-15. In some preferred embodiments the one or more biomarker is AB-40. In some preferred embodiments the one or more biomarker is ALP. In some preferred embodiments the one or more biomarker is MCP-3. In some preferred embodiments the one or more biomarker is SOD-1. In some preferred embodiments the one or more biomarker is BMP-9. In some preferred embodiments the one or more biomarker is IL-8. In some preferred embodiments the one or more biomarker is MIP-1 beta. In some preferred embodiments the one or more biomarker is RAGE. In some preferred embodiments the one or more biomarker is MPO. In some preferred embodiments the one or more biomarker is CgA. In some preferred embodiments the one or more biomarker is HE4. In some preferred embodiments the one or more biomarker is ADM. In some preferred embodiments the one or more biomarker is B2M. In some preferred embodiments the one or more biomarker is VDBP. In some preferred embodiments the one or more biomarker is Cystatin-C. In some preferred embodiments the one or more biomarker is Hepsin. In some preferred embodiments the one or more biomarker is LDH. In some preferred embodiments the one or more biomarker is Axl.

In some embodiments the use is as a biomarker in a method as defined in accordance with the above described first and second aspects of this facet of the disclosure.

Methods of Treating a Subject

A eighth aspect of this facet of the present disclosure relates to methods of treating a subject having, suspected of having, or diagnosed with non-small cell lung cancer (NSCLC). In some embodiments the method comprises: identifying a subject for treatment using a predictive method defined in accordance with the above described first and second aspects of this facet of the disclosure; and, administering to the subject a therapeutically effective amount of an agent capable of inhibiting or reversing EMT or a chemotherapeutic agent.

Accordingly, in some embodiments, the method comprises: (a) obtaining a sample from the subject; (b) determining if the subject is susceptible to treatment with an agent capable of inhibiting or reversing EMT or a chemotherapeutic agent using a predictive method defined in accordance with the above described first and second aspects of this facet of the disclosure; and, (c) administering to the subject a therapeutically effective amount of an agent capable of inhibiting or reversing EMT or a chemotherapeutic agent.

In some embodiments the method comprises administering a therapeutically effective amount of an agent capable of inhibiting or reversing EMT or chemotherapeutic agent to a subject identified as susceptible to said treatment using a predictive method defined in accordance with the above described first and second aspects of this facet of the disclosure.

In other embodiments the method comprises: (a) administering to a subject a therapeutically effective amount of an agent capable of inhibiting or reversing EMT or a chemotherapeutic agent; and, (b) determining if the subject is susceptible to said treatment using a predictive method defined in accordance with the above described second aspect of this facet of the disclosure. In some embodiments the method further comprises: (c) administering to the subject one or more further therapeutically effective amounts of an agent capable of inhibiting or reversing EMT or a chemotherapeutic agent provided that the subject is identified as susceptible to said treatment.

A ninth aspect of this facet of the present disclosure relates to an agent capable of inhibiting or reversing EMT or chemotherapeutic agent for use in a method of treating a subject having, suspected of having, or diagnosed with non-small cell lung cancer (NSCLC). In some embodiments the method of treatment is a method as defined above.

A tenth aspect of this facet of the present disclosure relates to use of an agent capable of inhibiting or reversing EMT or chemotherapeutic agent for use in a method of manufacture of a medicament for use in a method of treating a subject having, suspected of having, or diagnosed with non-small cell lung cancer (NSCLC). In some embodiments the method of treatment is a method as defined above.

In some embodiments of these methods, agents for use, or uses, the agent capable of inhibiting or reversing EMT or chemotherapeutic agent comprises an Axl inhibitor, Akt3 inhibitor, Slfn11 inhibitor, or PHGDH inhibitor. In some preferred embodiments the agent capable of inhibiting or reversing EMT or chemotherapeutic agent is an Axl inhibitor, for example BGB324/R428/bemcentinib, cabozantinib, TP-0903, foretinib, merestinib, bosutinib, gilteritinib, crizotinib, amuvatinib, sunitinib, sitravatinib, LDC1267, or UNC2025. Other Axl inhibitors include the anti-Axl antibodies described in WO2015/193428, WO2015/193430, WO2016/097370, and WO2016/166296. In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In some other preferred embodiments, the treatment comprises an Akt3 inhibitor. In some embodiments the Akt3 inhibitor is an Akt3 inhibitor disclosed in WO2016/102672.

In some embodiments of these methods, agents for use, or uses, the treatment is administered as a single agent. In other embodiments, the treatment is administered in combination with a further cancer treatment. Suitable further cancer treatments are outlined in detail above.

In some preferred embodiments the further cancer treatment is a PD-1 inhibitor or PD-L1 inhibitor. In some embodiments the further cancer treatment is a monoclonal antibody such as pembrolizumab, alemtuzumab, bevacizumab, cetuximab, ibritumomab, tiuxetan, or rituximab. In some preferred embodiments the further cancer treatment is a microtubule polymer stabilizer such as the taxanes paclitaxel and docetaxel. In some preferred embodiments the further cancer treatment is a tyrosine kinase inhibitor such as erlotinib hydrochloride, gefitinib, imatinib mesylate, and semaxanib. In some particularly preferred embodiments the further cancer treatment is pembrolizumab. In other particularly preferred embodiments the further cancer treatment is docetaxel. In yet other particularly preferred embodiments the further cancer treatment is erlotinib.

Statements of Disclosure

The following numbered statements relate to aspects of this facet of the present disclosure, and form part of the description:
101. A method of predicting a cancer-related outcome in a subject, the method comprising assessing the activity, expression, or amount of one or more biomarker in the subject, or in a sample derived from the subject;
   wherein the one or more biomarker is selected from the group consisting of: NT proBNP, Endostatin, IGFBP-2, VEGF-D, GDF-15, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl; and
   wherein the subject has, is suspected of having, or has been diagnosed with non-small cell lung cancer (NSCLC).
102. A method according to statement 101, wherein the method comprises:
   assessing the activity, expression, or amount of one or more biomarker in the subject, or in a sample derived from the subject, to obtain a sample profile of the one or more biomarker; and
   making a prediction based on the sample profile of the one or more biomarker.
103. A method according to statement 102, wherein the prediction is made by comparing the sample profile to a control profile.
104. A method according to statement 103, wherein the sample profile is obtained before the subject is contacted with or administered an agent capable of inhibiting or reversing EMT or a chemotherapeutic agent.
105. A method according to statement 104, wherein the control profile is:
   (i) obtained from a population of control subjects having NSCLC; or
   (ii) obtained from a control subject or population of control subjects having NSCLC and previously found to lack susceptibility to treatment with an agent capable of inhibiting or reversing EMT or a chemotherapeutic agent (that is, non-responder subjects);
   (iii) a predetermined profile of biomarker expression, activity, or amount values, for example a profile of “average, median, or mean” values or “standard ranges” of values, obtained from a control subject or population of control subjects having NSCLC and previously found to lack susceptibility to treatment with an agent capable of inhibiting or reversing EMT or a chemotherapeutic agent (that is, non-responder subjects);
   (iv) obtained from a control sample having a known “average, median, or mean” value of biomarkers indicative of a control subject or population of control subjects having NSCLC and previously found to lack susceptibility to treatment with an agent capable of inhibiting or reversing EMT or a chemotherapeutic agent (that is, non-responder subjects);
   (v) a predetermined profile of biomarker expression, activity, or amount “threshold” values obtained from a control subject or population of control subjects having NSCLC and previously found to be susceptible to treatment with an agent capable of inhibiting or reversing EMT or a chemotherapeutic agent (that is, responder subjects); or
   (vi) obtained from a control sample having a known “threshold” value of biomarkers indicative of a control subject or population of control subjects having NSCLC and previously found to be susceptible to treatment with an agent capable of inhibiting or reversing EMT or a chemotherapeutic agent (that is, responder subjects).
106. A method according to statement 105, wherein a higher activity, expression, or amount of one or more biomarker in the sample profile as compared with the control profile is indicative of susceptibility to treatment with an agent capable of inhibiting or reversing EMT or a chemotherapeutic agent.
107. A method according to statement 105, wherein a lower activity, expression, or amount of one or more biomarker in the sample profile as compared with the control profile is indicative of susceptibility to treatment with an agent capable of inhibiting or reversing EMT or a chemotherapeutic agent.
108. A method according to any one of statements 105-107, wherein the one or more biomarker includes at least one of: MCP-3, SOD-1, BMP-9, IL-8, MIP-1 beta, MPO, VDBP and/or Axl,
   and wherein a higher activity, expression, or amount of one or more of said biomarkers in the sample profile as compared with the control profile is indicative of susceptibility to treatment with an agent capable of inhibiting or reversing EMT or a chemotherapeutic agent.
109. A method according to any one of statements 105-108, wherein the one or more biomarker includes at least one of: NT proBNP, Endostatin, IGFBP-2, VEGF-D, GDF-15, AB-40, ALP, RAGE, CgA, HE4, ADM, B2M, Cystatin-C, Hepsin, and/or LDH,
   and wherein a lower activity, expression, or amount of one or more of said biomarkers in the sample profile as compared with the control profile is indicative of susceptibility to treatment with an agent capable of inhibiting or reversing EMT or a chemotherapeutic agent.
110. A method according to any one of statements 104-109, wherein:
   a lower activity, expression, or amount of NT proBNP and/or
   a lower activity, expression, or amount of Endostatin; and/or
   a lower activity, expression, or amount of IGFBP-2; and/or
   a lower activity, expression, or amount of VEGF-D; and/or
   a lower activity, expression, or amount of GDF-15; and/or
   a lower activity, expression, or amount of AB-40; and/or
   a lower activity, expression, or amount of ALP; and/or
   a lower activity, expression, or amount of RAGE; and/or
   a lower activity, expression, or amount of CgA; and/or
   a lower activity, expression, or amount of HE4; and/or
   a lower activity, expression, or amount of ADM; and/or
   a lower activity, expression, or amount of B2M; and/or
   a lower activity, expression, or amount of Cystatin-C; and/or
   a lower activity, expression, or amount of Hepsin; and/or
   a lower activity, expression, or amount of LDH; and/or
   a higher activity, expression, or amount of MCP-3; and/or
   a higher activity, expression, or amount of SOD-1; and/or
   a higher activity, expression, or amount of BMP-9; and/or
   a higher activity, expression, or amount of IL-8; and/or
   a higher activity, expression, or amount of MIP-1 beta, and/or
   a higher activity, expression, or amount of MPO; and/or
   a higher activity, expression, or amount of VDBP; and/or
   a higher activity, expression, or amount of Axl;
   in the sample profile as compared with the control profile is indicative of susceptibility to treatment with an agent capable of inhibiting or reversing EMT or a chemotherapeutic agent.
111. A method according to any one of statements 101-110, wherein the one or more biomarker comprises NT proBNP,
   optionally wherein the one or more biomarker comprises at least 1, 2, 3, 4, 5, 6, 7, 8, 9, 10, or more further biomarkers selected from: Endostatin, IGFBP-2, VEGF-D, GDF-15, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl.
112. A method according to any one of statements 101-110, wherein the one or more biomarker comprises Endostatin,
   optionally wherein the one or more biomarker comprises at least 1, 2, 3, 4, 5, 6, 7, 8, 9, 10, or more further biomarkers selected from: NT proBNP, IGFBP-2, VEGF-D, GDF-15, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl.
113. A method according to any one of statements 101-110, wherein the one or more biomarker comprises IGFBP-2,
   optionally wherein the one or more biomarker comprises at least 1, 2, 3, 4, 5, 6, 7, 8, 9, 10, or more further biomarkers selected from: NT proBNP, Endostatin, VEGF-D, GDF-15, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl.
114. A method according to any one of statements 101-110, wherein the one or more biomarker comprises VEGF-D,
   optionally wherein the one or more biomarker comprises at least 1, 2, 3, 4, 5, 6, 7, 8, 9, 10, or more further biomarkers selected from: NT proBNP, Endostatin, IGFBP-2, GDF-15, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl.
115. A method according to any one of statements 101-110, wherein the one or more biomarker comprises GDF-15,
   optionally wherein the one or more biomarker comprises at least 1, 2, 3, 4, 5, 6, 7, 8, 9, 10, or more further biomarkers selected from: NT proBNP, Endostatin, IGFBP-2, VEGF-D, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl.
116. A method according to any one of statements 101-110, wherein the one or more biomarker comprises AB-40,
   optionally wherein the one or more biomarker comprises at least 1, 2, 3, 4, 5, 6, 7, 8, 9, 10, or more further biomarkers selected from: NT proBNP, Endostatin, IGFBP-2, VEGF-D, GDF-15, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl.
117. A method according to any one of statements 101-110, wherein the one or more biomarker comprises ALP,
   optionally wherein the one or more biomarker comprises at least 1, 2, 3, 4, 5, 6, 7, 8, 9, 10, or more further biomarkers selected from: NT proBNP, Endostatin, IGFBP-2, VEGF-D, GDF-15, AB-40, MCP-3, SOD-1, BMP-9, IL-8, MIP-1 beta, RAGE, MPO, CgA, HE4, ADM, B2M, VDBP, Cystatin-C, Hepsin, LDH, and/or Axl.
118. A method according to any one of statements 101-110, wherein the one or more biomarker comprises MCP-3,
   optionally wherein the one or more biomarker comprises at least 1, 2, 3, 4, 5, 6, 7, 8, 9, 10, or more further biomarkers selected from: NT proBNP, Endostatin, IGFBP-2, VEGF-D, GDF-15, AB-40, ALP, SOD-1, BMP-9, IL-8, MIP-1 beta, RAGE, MPO, CgA, HE4, ADM, B2M, VDBP, Cystatin-C, Hepsin, LDH, and/or Axl.
119. A method according to any one of statements 101-110, wherein the one or more biomarker comprises SOD-1,
   optionally wherein the one or more biomarker comprises at least 1, 2, 3, 4, 5, 6, 7, 8, 9, 10, or more further biomarkers selected from: NT proBNP, Endostatin, IGFBP-2, VEGF-D, GDF-15, AB-40, ALP, MCP-3, BMP-9, IL-8, MIP-1 beta, RAGE, MPO, CgA, HE4, ADM, B2M, VDBP, Cystatin-C, Hepsin, LDH, and/or Axl.
120. A method according to any one of statements 101-110, wherein the one or more biomarker comprises BMP-9,
   optionally wherein the one or more biomarker comprises at least 1, 2, 3, 4, 5, 6, 7, 8, 9, 10, or more further biomarkers selected from: NT proBNP, Endostatin, IGFBP-2, VEGF-D, GDF-15, AB-40, ALP, MCP-3, SOD-1, IL-8, MIP-1 beta, RAGE, MPO, CgA, HE4, ADM, B2M, VDBP, Cystatin-C, Hepsin, LDH, and/or Axl.
121. A method according to any one of statements 101-110, wherein the one or more biomarker comprises IL-8,
   optionally wherein the one or more biomarker comprises at least 1, 2, 3, 4, 5, 6, 7, 8, 9, 10, or more further biomarkers selected from: NT proBNP, Endostatin, IGFBP-2, VEGF-D, GDF-15, AB-40, ALP, MCP-3, SOD-1, BMP-9, MIP-1 beta, RAGE, MPO, CgA, HE4, ADM, B2M, VDBP, Cystatin-C, Hepsin, LDH, and/or Axl.
122. A method according to any one of statements 101-110, wherein the one or more biomarker comprises MIP-1 beta,
   optionally wherein the one or more biomarker comprises at least 1, 2, 3, 4, 5, 6, 7, 8, 9, 10, or more further biomarkers selected from: NT proBNP, Endostatin, IGFBP-2, VEGF-D, GDF-15, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl.
123. A method according to any one of statements 101-110, wherein the one or more biomarker comprises RAGE,
   optionally wherein the one or more biomarker comprises at least 1, 2, 3, 4, 5, 6, 7, 8, 9, 10, or more further biomarkers selected from: NT proBNP, Endostatin, IGFBP-2, VEGF-D, GDF-15, AB-40, ALP, MCP-3, SOD-1, BMP-9, IL-8, MIP-1 beta, MPO, CgA, HE4, ADM, B2M, VDBP, Cystatin-C, Hepsin, LDH, and/or Axl.
124. A method according to any one of statements 101-110, wherein the one or more biomarker comprises MPO,
   optionally wherein the one or more biomarker comprises at least 1, 2, 3, 4, 5, 6, 7, 8, 9, 10, or more further biomarkers selected from: NT proBNP, Endostatin, IGFBP-2, VEGF-D, GDF-15, AB-40, ALP, MCP-3, SOD-1, BMP-9, IL-8, MIP-1 beta, RAGE, CgA, HE4, ADM, B2M, VDBP, Cystatin-C, Hepsin, LDH, and/or Axl.
125. A method according to any one of statements 101-110, wherein the one or more biomarker comprises CgA,
   optionally wherein the one or more biomarker comprises at least 1, 2, 3, 4, 5, 6, 7, 8, 9, 10, or more further biomarkers selected from: NT proBNP, Endostatin, IGFBP-2, VEGF-D, GDF-15, AB-40, ALP, MCP-3, SOD-1, BMP-9, IL-8, MIP-1 beta, RAGE, MPO, HE4, ADM, B2M, VDBP, Cystatin-C, Hepsin, LDH, and/or Axl.
126. A method according to any one of statements 101-110, wherein the one or more biomarker comprises HE4,
   optionally wherein the one or more biomarker comprises at least 1, 2, 3, 4, 5, 6, 7, 8, 9, 10, or more further biomarkers selected from: NT proBNP, Endostatin, IGFBP-2, VEGF-D, GDF-15, AB-40, ALP, MCP-3, SOD-1, BMP-9, IL-8, MIP-1 beta, RAGE, MPO, CgA, ADM, B2M, VDBP, Cystatin-C, Hepsin, LDH, and/or Axl.
127. A method according to any one of statements 101-110, wherein the one or more biomarker comprises ADM,
   optionally wherein the one or more biomarker comprises at least 1, 2, 3, 4, 5, 6, 7, 8, 9, 10, or more further biomarkers selected from: NT proBNP, Endostatin, IGFBP-2, VEGF-D, GDF-15, AB-40, ALP, MCP-3, SOD-1, BMP-9, IL-8, MIP-1 beta, RAGE, MPO, CgA, HE4, B2M, VDBP, Cystatin-C, Hepsin, LDH, and/or Axl.
128. A method according to any one of statements 101-110, wherein the one or more biomarker comprises B2M,
   optionally wherein the one or more biomarker comprises at least 1, 2, 3, 4, 5, 6, 7, 8, 9, 10, or more further biomarkers selected from: NT proBNP, Endostatin, IGFBP-2, VEGF-D, GDF-15, AB-40, ALP, MCP-3, SOD-1, BMP-9, IL-8, MIP-1 beta, RAGE, MPO, CgA, HE4, ADM, VDBP, Cystatin-C, Hepsin, LDH, and/or Axl.
129. A method according to any one of statements 101-110, wherein the one or more biomarker comprises VDBP,
   optionally wherein the one or more biomarker comprises at least 1, 2, 3, 4, 5, 6, 7, 8, 9, 10, or more further biomarkers selected from: NT proBNP, Endostatin, IGFBP-2, VEGF-D, GDF-15, AB-40, ALP, MCP-3, SOD-1, BMP-9, IL-8, MIP-1 beta, RAGE, MPO, CgA, HE4, ADM, B2M, Cystatin-C, Hepsin, LDH, and/or Axl.
130. A method according to any one of statements 101-110, wherein the one or more biomarker comprises Cystain-C,
   optionally wherein the one or more biomarker comprises at least 1, 2, 3, 4, 5, 6, 7, 8, 9, 10, or more further biomarkers selected from: NT proBNP, Endostatin, IGFBP-2, VEGF-D, GDF-15, AB-40, ALP, MCP-3, SOD-1, BMP-9, IL-8, MIP-1 beta, RAGE, MPO, CgA, HE4, ADM, B2M, VDBP, Hepsin, LDH, and/or Axl.
131. A method according to any one of statements 101-110, wherein the one or more biomarker comprises Hepsin,
   optionally wherein the one or more biomarker comprises at least 1, 2, 3, 4, 5, 6, 7, 8, 9, 10, or more further biomarkers selected from: NT proBNP, Endostatin, IGFBP-2, VEGF-D, GDF-15, AB-40, ALP, MCP-3, SOD-1, BMP-9, IL-8, MIP-1 beta, RAGE, MPO, CgA, HE4, ADM, B2M, VDBP, Cystatin-C, LDH, and/or Axl.
132. A method according to any one of statements 101-110, wherein the one or more biomarker comprises LDH,
   optionally wherein the one or more biomarker comprises at least 1, 2, 3, 4, 5, 6, 7, 8, 9, 10, or more further biomarkers selected from: NT proBNP, Endostatin, IGFBP-2, VEGF-D, GDF-15, AB-40, ALP, MCP-3, SOD-1, BMP-9, IL-8, MIP-1 beta, RAGE, MPO, CgA, HE4, ADM, B2M, VDBP, Cystatin-C, Hepsin, and/or Axl.
133. A method according to any one of statements 101-110, wherein the one or more biomarker comprises Axl,
   optionally wherein the one or more biomarker comprises at least 1, 2, 3, 4, 5, 6, 7, 8, 9, 10, or more further biomarkers selected from: NT proBNP, Endostatin, IGFBP-2, VEGF-D, GDF-15, AB-40, ALP, MCP-3, SOD-1, BMP-9, IL-8, MIP-1 beta, RAGE, MPO, CgA, HE4, ADM, B2M, VDBP, Cystatin-C, Hepsin, and/or LDH.
134. A method according to any one of statements 111-133, wherein the one or more biomarker further comprises NT proBNP.
135. A method according to any one of statements 111-134, wherein the one or more biomarker further comprises Endostatin.
136. A method according to any one of statements 111-135, wherein the one or more biomarker further comprises IGFBP-2.
137. A method according to any one of statements 111-136, wherein the one or more biomarker further comprises VEGF-D.
138. A method according to any one of statements 111-137, wherein the one or more biomarker further comprises GDF-15.
139. A method according to any one of statements 111-138, wherein the one or more biomarker further comprises AB-40.
140. A method according to any one of statements 111-139, wherein the one or more biomarker further comprises ALP.
141. A method according to any one of statements 111-140, wherein the one or more biomarker further comprises MCP-3.
142. A method according to any one of statements 111-141, wherein the one or more biomarker further comprises SOD-1.
143. A method according to any one of statements 111-142, wherein the one or more biomarker further comprises BMP-9.
144. A method according to any one of statements 111-143, wherein the one or more biomarker further comprises IL-8.
145. A method according to any one of statements 111-144, wherein the one or more biomarker further comprises MIP-1 beta.
146. A method according to any one of statements 111-145, wherein the one or more biomarker further comprises RAGE.
147. A method according to any one of statements 111-146, wherein the one or more biomarker further comprises MPO.
148. A method according to any one of statements 111-147, wherein the one or more biomarker further comprises CgA.
149. A method according to any one of statements 111-148, wherein the one or more biomarker further comprises HE4.
150. A method according to any one of statements 111-149, wherein the one or more biomarker further comprises ADM.
151. A method according to any one of statements 111-150, wherein the one or more biomarker further comprises B2M.
152. A method according to any one of statements 111-151, wherein the one or more biomarker further comprises VDBP.
153. A method according to any one of statements 111-152, wherein the one or more biomarker further comprises Cystatin-C.
154. A method according to any one of statements 111-153, wherein the one or more biomarker further comprises Hepsin.
155. A method according to any one of statements 111-154, wherein the one or more biomarker further comprises LDH.
156. A method according to any one of statements 111-155, wherein the one or more biomarker further comprises Axl.
157. A method according to any one of statements 101-156, wherein:
   the Axl biomarker comprises an amino acid sequence having at least 70%, 80%, 90%, 95%, 99% or 100% sequence identity with the full-length of the amino acid sequence disclosed at UniProt accession no: P30530-1 or P30530-2 (entry version 196) or a fragment thereof;
   the N-terminal prohormone of brain natriuretic peptide (NT proBNP) biomarker comprises an amino acid sequence having at least 70%, 80%, 90%, 95%, 99% or 100% sequence identity with the full-length of the amino acid sequence disclosed at UniProt accession no: P16860-1 (entry version 186) or a fragment thereof;
   the Endostatin biomarker comprises an amino acid sequence having at least 70%, 80%, 90%, 95%, 99% or 100% sequence identity with the full-length of the amino acid sequence disclosed at UniProt accession no: P39059-1 (entry version 170) or a fragment thereof;
   the Insulin-like Growth Factor-Binding Protein 2 (IGFBP-2) biomarker comprises an amino acid sequence having at least 70%, 80%, 90%, 95%, 99% or 100% sequence identity with the full-length of the amino acid sequence disclosed at UniProt accession no: P18065-1 (entry version 193) or a fragment thereof;
   the Vascular endothelial growth factor D (VEGF-D) biomarker comprises an amino acid sequence having at least 70%, 80%, 90%, 95%, 99% or 100% sequence identity with the full-length of the amino acid sequence disclosed at UniProt accession no: 043915-1 (entry version 159) or a fragment thereof;
   the Growth/differentiation factor 15 (GDF-15) comprises an amino acid sequence having at least 70%, 80%, 90%, 95%, 99% or 100% sequence identity with the full-length of the amino acid sequence disclosed at UniProt accession no: Q99988-1 (entry version 166) or a fragment thereof;
   the Beta Amyloid 1-40 (AB-40) biomarker comprises an amino acid sequence having at least 70%, 80%, 90%, 95%, 99% or 100% sequence identity with the full-length of the amino acid sequence disclosed at UniProt accession no: P05067-1, P05067-2, P05067-3, P05067-4, P05067-5, P05067-6, P05067-7, P05067-8, P05067-9, P05067-10, or P05067-11 (entry version 271) or a fragment thereof;
   the Antileukoproteinase (ALP) biomarker comprises an amino acid sequence having at least 70%, 80%, 90%, 95%, 99% or 100% sequence identity with the full-length of the amino acid sequence disclosed at UniProt accession no: P03973 (entry version 184) or a fragment thereof;
   the Monocyte Chemotactic Protein 3 (MCP-3) comprises an amino acid sequence having at least 70%, 80%, 90%, 95%, 99% or 100% sequence identity with the full-length of the amino acid sequence disclosed at UniProt accession no: P80098-1 (entry version 169) or a fragment thereof;
   the Superoxide Dismutase 1, soluble (SOD-1) biomarker comprises an amino acid sequence having at least 70%, 80%, 90%, 95%, 99% or 100% sequence identity with the full-length of the amino acid sequence disclosed at UniProt accession no: P00441-1 (entry version 240) or a fragment thereof;
   the Bone morphogenetic protein 9 (BMP-9) biomarker comprises an amino acid sequence having at least 70%, 80%, 90%, 95%, 99% or 100% sequence identity with the full-length of the amino acid sequence disclosed at UniProt accession no: Q9UK05-1 (entry version 162) or a fragment thereof;
   the Interleukin-8 (IL-8) biomarker comprises an amino acid sequence having at least 70%, 80%, 90%, 95%, 99% or 100% sequence identity with the full-length of the amino acid sequence disclosed at UniProt accession no: P10145 (entry version 210) or a fragment thereof;
   the Macrophage Inflammatory Protein-1 beta (MIP-1 beta) biomarker comprises an amino acid sequence having at least 70%, 80%, 90%, 95%, 99% or 100% sequence identity with the full-length of the amino acid sequence disclosed at UniProt accession no: P13236 (entry version 187) or a fragment thereof;
   the Receptor for advanced glycosylation end products (RAGE) biomarker comprises an amino acid sequence having at least 70%, 80%, 90%, 95%, 99% or 100% sequence identity with the full-length of the amino acid sequence disclosed at UniProt accession no: Q15109-1, Q15109-2, Q15109-3, Q15109-4, Q15109-5, Q15109-6, Q15109-7, Q15109-8, Q15109-9, Q15109-10 (entry version 189) or a fragment thereof;
   the Myeloperoxidase (MPO) biomarker comprises an amino acid sequence having at least 70%, 80%, 90%, 95%, 99% or 100% sequence identity with the full-length of the amino acid sequence disclosed at UniProt accession no: P05164-1, P05164-2, or P05164-3 (entry version 217) or a fragment thereof;
   the Chromogranin-A (CgA) biomarker comprises an amino acid sequence having at least 70%, 80%, 90%, 95%, 99% or 100% sequence identity with the full-length of the amino acid sequence disclosed at UniProt accession no: P10645-1 (entry version 200) or a fragment thereof;
   the HE4 biomarker comprises an amino acid sequence having at least 70%, 80%, 90%, 95%, 99% or 100% sequence identity with the full-length of the amino acid sequence disclosed at UniProt accession no: Q14508-1, Q14508-2, Q14508-3, Q14508-4, or Q14508-5 (entry version 169) or a fragment thereof;
   the Adrenomedullin (ADM) biomarker comprises an amino acid sequence having at least 70%, 80%, 90%, 95%, 99% or 100% sequence identity with the full-length of the amino acid sequence disclosed at UniProt accession no: P35318-1 (entry version 174) or a fragment thereof;
   the Beta-2-Microglobulin (B2M) biomarker comprises an amino acid sequence having at least 70%, 80%, 90%, 95%, 99% or 100% sequence identity with the full-length of the amino acid sequence disclosed at UniProt accession no: P61769-1 (entry version 181) or a fragment thereof;
   the Vitamin D-Binding Protein (VDBP) biomarker comprises an amino acid sequence having at least 70%, 80%, 90%, 95%, 99% or 100% sequence identity with the full-length of the amino acid sequence disclosed at UniProt accession no: P02774-1, P02774-2, or P02774-3 (entry version 201) or a fragment thereof;
   the Cystatin-C biomarker comprises an amino acid sequence having at least 70%, 80%, 90%, 95%, 99% or 100% sequence identity with the full-length of the amino acid sequence disclosed at UniProt accession no: P01034-1 (entry version 213) or a fragment thereof;
   the Hepsin biomarker comprises an amino acid sequence having at least 70%, 80%, 90%, 95%, 99% or 100% sequence identity with the full-length of the amino acid sequence disclosed at UniProt accession no: P05981-1 (entry version 183) or a fragment thereof;
   the Lactate Dehydrogenase (LDH) biomarker comprises an amino acid sequence having at least 70%, 80%, 90%, 95%, 99% or 100% sequence identity with the full-length of the amino acid sequence disclosed at UniProt accession no: P00338-1, P00338-2, P00338-3, P00338-4, or P00338-5 (entry version 224) or a fragment thereof; and/or
   the Lactate Dehydrogenase (LDH) biomarker comprises an amino acid sequence having at least 70%, 80%, 90%, 95%, 99% or 100% sequence identity with the full-length of the amino acid sequence disclosed at UniProt accession no: P07195-1 (entry version 218) or a fragment thereof.
   or a nucleic acid (either DNA or RNA) having at least 70%, 80%, 90%, 95%, 99% or 100% sequence identity with the full-length nucleic acid sequence encoding said amino acid sequences.
158. A method according to any one of statement 101-157, wherein the method comprises determining the amount of at least 2, 3, 4, 5, 6, 7, 8, 9, 10, 11, 12, 13, 14, 15, 20 of said biomarkers in the subject, or sample derived from the subject.
159. A method according to any one of statements 104-158, wherein assessing the activity, expression, or amount of one or more biomarker in the subject, or in a sample derived from the subject, comprises:
   contacting a sample, or an extract from the sample, with at least 1, 2, 3, 4, 5, 6, 7, 8, 9, 10 or more reagents for detecting the activity, expression, or amount of one or more biomarkers, each of which is selective for a particular biomarker; and
   detecting and/or quantifying said reagents for detecting.
160. A method according to statement 159, wherein assessing the expression or amount of one or more biomarker in the subject, or in a sample derived from the subject, comprises:
   contacting a sample, or an extract from the sample, with at least 1, 2, 3, 4, 5, 6, 7, 8, 9, 10 or more specific binding members, each of which selectively binds to a respective biomarker protein or mRNA; and
   detecting and/or quantifying formation of complex formed by said specific binding member and said biomarker protein or mRNA.
161. A method according to statement 160 wherein the specific binding member comprises an antibody molecule or binding fragment thereof.
162. A method according to statement 160 or 161, wherein detecting formation of a complex formed by the specific binding member and the biomarker protein or mRNA is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.
163. A method according to any one of statements 160-162 wherein the expression or amount of the one or more biomarker is determined as the absolute amount of the respective biomarker protein or mRNA in the sample.
164. A method according to any one of statements 160-162 wherein the expression or amount of the one or more biomarker is determined as a relative amount of the respective biomarker protein or mRNA in the sample,
   optionally wherein the relative amount is determined relative to a reference protein or mRNA in the sample, the expression of which is not altered by NSCLC.
165. A method according to any one of statements 101-164, wherein the cancer-related outcome is susceptibility to treatment with an agent capable of inhibiting or reversing EMT or a chemotherapeutic agent.
166. A method according to statement 165, wherein the agent capable of inhibiting or reversing EMT or chemotherapeutic agent is an Axl inhibitor, Akt3 inhibitor, Slfn11 inhibitor, or PHGDH inhibitor.
167. A method according to statement 166, wherein the agent capable of inhibiting or reversing EMT or chemotherapeutic agent is an Axl inhibitor.
168. A method according to statement 167, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
169. A method according to statement 168, wherein the Axl inhibitor is BGB324/R428/bemcentinib.
170. A method according to statement 168, wherein the Axl inhibitor is an anti-Axl antibody described in WO2015/193428, WO2015/193430, WO2016/097370, or WO2016/166296.
171. A method according to statement 166, wherein the agent capable of inhibiting or reversing EMT or chemotherapeutic agent is an Akt3 inhibitor.
172. A method according to statement 171, wherein the Akt3 inhibitor is an Akt3 inhibitor disclosed in WO2016/102672.
173. A method according to any one of statements 165-172, wherein the agent capable of inhibiting or reversing EMT is administered as a single agent.
174. A method according to any one of statements 165-172, wherein the agent capable of inhibiting or reversing EMT is administered in combination with a further cancer treatment.
175. A method according to statement 174, wherein the further cancer treatment is selected from:
   (i) alkylating agents, including alkyl sulfonates such as busulfan;
   (ii) nitrogen mustards such as chlorambucil, cyclophosphamide, estramustine, ifosfamide, mechlorethamine, melphalan, and uramustine;
   (iii) ethyleneimine derivatives such as thiotepa;
   (iv) nitrosoureas such as carmustine, lomustine, and streptozocin;
   (v) triazenes such as dacarbazine, procarbazine, and temozolamide;
   (vi) platinum compounds such as cisplatin, carboplatin, oxaliplatin, satraplatin, and picoplatin onnaplatin, tetraplatin, sprioplatin, iproplatin, chloro(diethylenediamino)-platinum (II) chloride, dichloro(ethylenediamino)-platinum (II), diamino(2-ethylmalonato)platinum (II), (1,2-diaminocyclohexane)malonatoplatinum (II), (4-carboxyphthalo)-(1,2-diaminocyclohexane)platinum (II), (1,2-diaminocyclohexane)-(isocitrato)platinum (II), and (1,2-diaminocyclohexane)-cis-(pyruvato)platinum (II);
   (vii) antimetabolites, including antifolates such as methotrexate, permetrexed, raltitrexed, and trimetrexate;
   (viii) pyrimidine analogues such as azacitidine, capecitabine, cytarabine, edatrexate, floxuridine, fluorouracil, gemcitabine, and troxacitabine;
   (ix) purine analogues such as cladribine, chlorodeoxyadenosine, clofarabine, fludarabine, mercaptopurine, pentostatin, and thioguanine;
   (x) natural products, including antitumor antibiotics such as bleomycin, dactinomycin, mithramycin, mitomycin, mitoxantrone, porfiromycin;
   (xi) anthracyclines such as daunorubicin, doxorubicin, epirubicin, idarubicin, and valrubicin;
   (xii) mitotic inhibitors such as the vinca alkaloids vinblastine, vinvesir, vincristine, vindesine, and vinorelbine;
   (xiii) enzymes such as L-asparaginase and PEG-L-asparaginase;
   (xiv) microtubule polymer stabilizers such as the taxanes paclitaxel and docetaxel;
   (xv) topoisomerase I inhibitors such as the camptothecins irinotecan and topotecan;
   (xvi) topoisomerase II inhibitors such as podophyllotoxin, amsacrine, etoposide, teniposide, losoxantrone and actinomycin;
   (xvii) hormones and hormone antagonists, including
      androgens such as fluoxymesterone and testolactone;
      antiandrogens such as bicalutamide, cyproterone, flutamide, and nilutamide;
      corticosteroids such as dexamethasone and prednisone;
      aromatase inhibitors such as aminoglutethimide, anastrozole, exemestane, formestane, and letrozole;
      estrogens such as diethylstilbestrol;
      antiestrogens such as fulvestrant, raloxifene, tamoxifen, and toremifine;
      luteinising hormone-releasing hormone (LHRH) agonists and antagonists such as abarelix, buserelin, goserelin, leuprolide, histrelin, desorelin, nafarelin acetate and triptorelin;
      progestins such as medroxyprogesterone acetate and megestrol acetate; and
      thyroid hormones such as levothyroxine and liothyronine;
   (xviii) PKB pathway inhibitors, including perifosine, enzastaurin hydrochloride, and triciribine;
   (xix) P13K inhibitors such as semaphore and SF1126, and MTOR inhibitors such as rapamycin and analogues;
   (xx) CDK inhibitors, including seliciclib, alvocidib, and 7-hydroxystaurosporine;
   (xxi) COX-2 inhibitors, including celecoxib;
   (xxii) HDAC inhibitiors, including trichostatin A, suberoylanilide hydroxamic acid, and chlamydocin;
   (xxiii) DNA methylase inhibitors, including temozolomide, and miscellaneous agents, including altretamine, arsenic trioxide, thalidomide, lenalidomide, gallium nitrate, levamisole, mitotane, hydroxyurea, octreotide, procarbazine, suramin, photodynamic compounds such as methoxsalen and sodium porfimer;
   (xxiv) proteasome inhibitors such as bortezomib;
   (xxv) molecular targeted therapy agents including: functional therapeutic agents, including gene therapy agents and antisense therapy agents;
   (xxvi) tyrosine kinase inhibitors such as erlotinib hydrochloride, gefitinib, imatinib mesylate, and semaxanib;
   (xxvii) Raf inhibitors such as sorafenib, and gene expression modulators such as the retinoids and rexinoids, for example adapalene, bexarotene, trans-retinoic acid, 9-cis-retinoic acid, and N-(4-hydroxyphenyl)retinamide;
   (xxviii) phenotype-directed therapy agents, including monoclonal antibodies such as pembrolizumab, alemtuzumab, bevacizumab, cetuximab, ibritumomab tiuxetan, rituximab, and trastuzumab, immunotoxins such as gemtuzumab ozogamicin, radioimmunoconjugates such as I-tositumobab;
   (xxix) cancer vaccines;
   (xxx) biologic therapy agents including: interferons such as interferon-[alpha]2a and interferon-[alpha]2b, and interleukins such as aldesleukin, denileukin diftitox, and oprelvekin;
   (xxxi) anticancer therapies involving the use of protective or adjunctive agents, including: cytoprotective agents such as amifostine, and dexrazoxane, phosphonates such as pamidronate and zoledronic acid, and stimulating factors such as epoetin, darbeopetin, filgrastim, PEG-filgrastim, and sargramostim;
   (xxxii) Axl inhibitors such as 1-(6,7-dihydro-5H-benzo[6,7]cyclohepta[1,2-c]pyridazin-3-yl)-N3-((7-(S)-pyrrolidin-1-yl)-6,7,8,9-tetrahydro-5H-benzo[7]annulene-2-yl)-1H-1,2,4-triazole-3,5-diamine; and/or
   (xxxiii) further combination chemotherapeutic regimens, such as combinations of carboplatin/paclitaxel, capecitabine/docetaxel, fluorauracil/levamisole, fluorauracil/leucovorin, methotrexate/leucovorin, and trastuzumab/paclitaxel, alone or in further combination with carboplatin.
176. A method according to statement 129, wherein the further cancer treatment is:
   (i) a PD-1 inhibitor or PD-L1 inhibitor;
   (ii) a monoclonal antibody such as pembrolizumab, alemtuzumab, bevacizumab, cetuximab, ibritumomab, tiuxetan, or rituximab;
   (iii) a microtubule polymer stabilizer such as the taxanes paclitaxel and docetaxel; and/or
   (iv) a tyrosine kinase inhibitor such as erlotinib hydrochloride, gefitinib, imatinib mesylate, and semaxanib.
177. A method according to statement 176, wherein the further cancer treatment is pembrolizumab.
178. A method according to statement 176, wherein the further cancer treatment is docetaxel.
179. A method according to statement 176, wherein the further cancer treatment is erlotinib.
180. A method according to any one of statements 101-179, wherein the subject is mammalian, optionally wherein the subject is human.
181. A method according to any one of statements 101-180, wherein the predictive method is performed before the subject receives a treatment or course of treatment for NSCLC.
182. A method according to any one of statement 101-181, wherein the predictive method is performed during or after a treatment or course of treatment for NSCLC.
183. A method according to any one of statements 101-180 or 182, wherein the subject has previously been treated with an agent capable of inhibiting or reversing EMT or a chemotherapeutic agent.
184. A method according to statement 183, wherein the subject has previously been treated with an agent selected from: a PHGDH inhibitor, a Slfn11 inhibitor, an Axl inhibitor, or an Akt3 inhibitor.
185. A method according to any one of statements 183-184, wherein the subject has previously been treated with an agent that is different to the agent capable of inhibiting or reversing EMT or chemotherapeutic agent for which the subject's susceptibility is being predicted.
186. A method according to any one of statements 101-181, wherein the subject has not previously been treated with an agent capable of inhibiting or reversing EMT or a chemotherapeutic agent.
187. A method according to statement 186, wherein the subject has not previously been treated with an Axl inhibitor.
188. A method according to statement 186, wherein the subject has not previously been treated with an Akt3 inhibitor.
189. A method according to any one of statements 101-188, wherein the sample is selected from: blood, serum, plasma, tissue biopsy, culture supernatant, or urine.
190. A method according to statement 189, wherein the sample is a blood, serum, or plasma sample.
191. A method according to statement 190, wherein the sample is a serum sample.
192. A method according to any one of statements 104-191, wherein the method further comprises a subsequent step of assessing the activity, expression, or amount of one or more biomarker in the subject, in which a sample profile is obtained after contacting the subject with an agent capable of inhibiting or reversing EMT or a chemotherapeutic agent.
193. A method according to statement 103, wherein the sample profile is obtained after contacting the subject with an agent capable of inhibiting or reversing EMT or a chemotherapeutic agent.
194. A method according to statement 192 or 193, wherein the control profile is obtained from the same subject prior to contacting said subject with the agent capable of inhibiting or reversing EMT or a chemotherapeutic agent.
195. A method according to statement 194, wherein an increase in the activity, expression, or amount of one or more biomarker in the sample profile as compared with the control profile is indicative of higher susceptibility to treatment with an agent capable of inhibiting or reversing EMT or a chemotherapeutic agent.
196. A method according to statement 194, wherein a decrease in the activity, expression, or amount of one or more biomarker in the sample profile as compared with the control profile is indicative of higher susceptibility to treatment with an agent capable of inhibiting or reversing EMT or a chemotherapeutic agent.
197. A method according to statement 194, wherein an increase in the activity, expression, or amount of one or more biomarker in the sample profile as compared with the control profile is indicative of lower susceptibility to treatment with an agent capable of inhibiting or reversing EMT or a chemotherapeutic agent.
198. A method according to statement 194, wherein a decrease in the activity, expression, or amount of one or more biomarker in the sample profile as compared with the control profile is indicative of lower susceptibility to treatment with an agent capable of inhibiting or reversing EMT or a chemotherapeutic agent.
199. A method according to statement 195, wherein the one or more biomarker includes at least Axl.
200. A method according to statement 195, wherein the one or more biomarker includes at least NT proBNP.
201. A method according to statement 195, wherein the one or more biomarker includes at least Endostatin.
202. A method according to statement 195, wherein the one or more biomarker includes at least IGFBP-2.
203. A method according to statement 195, wherein the one or more biomarker includes at least VEGF-D.
204. A method according to statement 195, wherein the one or more biomarker includes at least GDF-15.
205. A method according to statement 195, wherein the one or more biomarker includes at least AB-40.
206. A method according to statement 195, wherein the one or more biomarker includes at least ALP.
207. A method according to statement 195, wherein the one or more biomarker includes at least MCP-3.
208. A method according to statement 195, wherein the one or more biomarker includes at least SOD-1.
209. A method according to statement 195, wherein the one or more biomarker includes at least BMP-9.
210. A method according to statement 195, wherein the one or more biomarker includes at least IL-8.
211. A method according to statement 195, wherein the one or more biomarker includes at least MIP-1 beta.
212. A method according to statement 195, wherein the one or more biomarker includes at least RAGE
213. A method according to statement 195, wherein the one or more biomarker includes at least MPO.
214. A method according to statement 195, wherein the one or more biomarker includes at least CgA.
215. A method according to statement 195, wherein the one or more biomarker includes at least HE4.
216. A method according to statement 195, wherein the one or more biomarker includes at least ADM.
217. A method according to statement 195, wherein the one or more biomarker includes at least B2M.
218. A method according to statement 195, wherein the one or more biomarker includes at least VDBP.
219. A method according to statement 195, wherein the one or more biomarker includes at least Cystatin-C.
220. A method according to statement 195, wherein the one or more biomarker includes at least Hepsin.
221. A method according to statement 195, wherein the one or more biomarker includes at least LDH.
222. A method according to any one of statements 192-221, wherein assessing the activity, expression, or amount of one or more biomarker in the subject, or in a sample derived from the subject, comprises:
   contacting a sample, or an extract from the sample, with at least 1, 2, 3, 4, 5, 6, 7, 8, 9, 10 or more reagents for detecting the activity, expression, or amount of one or more biomarkers, each of which is selective for a particular biomarker; and
   detecting and/or quantifying said reagents for detecting.
223. A method according to statement 222, wherein assessing the expression or amount of one or more biomarker in the subject, or in a sample derived from the subject, comprises:
   contacting a sample, or an extract from the sample, with at least 1, 2, 3, 4, 5, 6, 7, 8, 9, 10 or more specific binding members, each of which selectively binds to a respective biomarker protein or mRNA; and
   detecting and/or quantifying formation of a complex formed by said specific binding member and said biomarker protein or mRNA.
224. A method according to statement 223, wherein the specific binding member comprises an antibody molecule or binding fragment thereof.
225. A method according to statement 223 or 224, wherein detecting formation of a complex formed by the specific binding member and the biomarker protein or mRNA is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.
226. A method according to any one of statements 223-225 wherein the expression or amount of the one or more biomarker is determined as the absolute amount of the respective biomarker protein or mRNA in the sample.
227. A method according to any one of statements 223-225 wherein the expression or amount of the one or more biomarker is determined as a relative amount of the respective biomarker protein or mRNA in the sample,
   optionally wherein the relative amount is determined relative to a reference protein or mRNA in the sample, the expression of which is not altered by NSCLC.
228. A method according to any one of statements 196-227, wherein the agent capable of inhibiting or reversing EMT or chemotherapeutic agent is an Axl inhibitor, Akt3 inhibitor, Slfn11 inhibitor, or PHGDH inhibitor.
229. A method according to statement 228, wherein the agent capable of inhibiting or reversing EMT or chemotherapeutic agent is an Axl inhibitor.
230. A method according to statement 229, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
231. A method according to statement 230, wherein the Axl inhibitor is BGB324/R428/bemcentinib.
232. A method according to statement 230, wherein the Axl inhibitor is an anti-Axl antibody described in WO2015/193428, WO2015/193430, WO2016/097370, or WO2016/166296.
233. A method according to statement 228, wherein the agent capable of inhibiting or reversing EMT or chemotherapeutic agent is an Akt3 inhibitor.
234. A method according to statement 233, wherein the Akt3 inhibitor is an Akt3 inhibitor disclosed in WO2016/102672.
235. A method according to any one of statements 228-234, wherein the agent capable of inhibiting or reversing EMT is administered as a single agent.
236. A method according to any one of statements 228-234, wherein the agent capable of inhibiting or reversing EMT is administered in combination with a further cancer treatment.
237. A method according to statement 236, wherein the further cancer treatment is selected from the further cancer treatments listed in statement 175.
238. A method according to statement 236, wherein the further cancer treatment is:
   (i) a PD-1 inhibitor or PD-L1 inhibitor;
   (ii) a monoclonal antibody such as pembrolizumab, alemtuzumab, bevacizumab, cetuximab, ibritumomab, tiuxetan, or rituximab;
   (iii) a microtubule polymer stabilizer such as the taxanes paclitaxel and docetaxel; and/or
   (iv) a tyrosine kinase inhibitor such as erlotinib hydrochloride, gefitinib, imatinib mesylate, and semaxanib.
239. A method according to statement 238, wherein the further cancer treatment is pembrolizumab.
240. A method according to statement 238, wherein the further cancer treatment is docetaxel.
241. A method according to statement 238, wherein the further cancer treatment is erlotinib.
242. A method according to any one of statements 193-241, wherein the subject is mammalian, optionally wherein the subject is human.
243. A method according to any one of statements 193-242, wherein the subject was selected for treatment with the agent capable of inhibiting or reversing EMT or a chemotherapeutic agent, using a predictive method according to any one of statements 101-192.
244. A method according to any one of statements 193-243, wherein the sample is selected from: blood, serum, plasma, tissue biopsy, culture supernatant, or urine.
245. A method according to statement 244, wherein the sample is a blood, serum, or plasma sample.
246. A method according to statement 245, wherein the sample is a serum sample.
247. A method according to any one of statements 1-246, wherein the method is performed in vitro or ex vivo.
201a. A method of selecting subjects having, suspected of having, or diagnosed with non-small cell lung cancer (NSCLC) for treatment with an agent capable of inhibiting or reversing EMT or a chemotherapeutic agent, the method comprising:
   identifying subjects susceptible to treatment with an agent capable of inhibiting or reversing EMT or a chemotherapeutic agent using a method according to any one of statements 101-247; and
   selecting thus identified subjects for treatment.
202a. A method of selecting subjects having, suspected of having, or diagnosed with non-small cell lung cancer (NSCLC) for continued treatment with an agent capable of inhibiting or reversing EMT or a chemotherapeutic agent, the method comprising:
   identifying subjects susceptible to treatment with an agent capable of inhibiting or reversing EMT or a chemotherapeutic agent using a method according to any one of statements 193-247; and
   selecting thus identified subjects for treatment.
203a. A method according to any one of statements 201a-202a, wherein treatment comprises administering to the subject a therapeutically effective amount of an agent capable of inhibiting or reversing EMT or a chemotherapeutic agent.
204a. A method according to statement 203a, wherein the treatment comprises an Axl inhibitor, Akt3 inhibitor, Slfn11 inhibitor, or PHGDH inhibitor.
205a. A method according to statement 204a, wherein the treatment comprises an Axl inhibitor.
206a. A method according to statement 205a, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
207a. A method according to statement 206a, wherein the Axl inhibitor is BGB324/R428/bemcentinib.
208a. A method according to statement 206a, wherein the Axl inhibitor is an anti-Axl antibody described in WO2015/193428, WO2015/193430, WO2016/097370, or WO2016/166296.
209a. A method according to statement 204a, wherein the treatment comprises an Akt3 inhibitor.
210a. A method according to statement 209a, wherein the Akt3 inhibitor is an Akt3 inhibitor disclosed in WO2016/102672.
211a. A method according to any one of statements 203a-210a, wherein the treatment is administered as a single agent.
212a. A method according to any one of statements 203a-211a, wherein the treatment is administered in combination with a further cancer treatment.
213a. A method according to statement 212a, wherein the further cancer treatment is selected from the further cancer treatments listed in statement 175.
214a. A method according to statement 212a, wherein the further cancer treatment is:
   (i) a PD-1 inhibitor or PD-L1 inhibitor;
   (ii) a monoclonal antibody such as pembrolizumab, alemtuzumab, bevacizumab, cetuximab, ibritumomab, tiuxetan, or rituximab;
   (iii) a microtubule polymer stabilizer such as the taxanes paclitaxel and docetaxel; and/or
   (iv) a tyrosine kinase inhibitor such as erlotinib hydrochloride, gefitinib, imatinib mesylate, and semaxanib.
215a. A method according to statement 214a, wherein the further cancer treatment is pembrolizumab.
216a. A method according to statement 214a, wherein the further cancer treatment is docetaxel.
217a. A method according to statement 214a, wherein the further cancer treatment is erlotinib.
301. A diagnostic kit comprising 1, 2, 3, 4, 5, 6, 7, 8, 9, 10 or more reagents for detecting the activity, expression, or amount of one or more biomarkers, each of which is selective for a particular biomarker selected from the group consisting of: NT proBNP, Endostatin, IGFBP-2, VEGF-D, GDF-15, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl.
302. A test device comprising 1, 2, 3, 4, 5, 6, 7, 8, 9, 10 or more reagents for detecting the activity, expression, or amount of one or more biomarkers, each of which is selective for a particular biomarker selected from the group consisting of: NT proBNP, Endostatin, IGFBP-2, VEGF-D, GDF-15, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl.
303. A diagnostic kit or test device according to any one of statements 301 or 302, comprising:
   1, 2, 3, 4, 5, 6, 7, 8, 9, 10 or more specific binding members, each of which selectively binds to a biomarker selected from the group consisting of: NT proBNP, Endostatin, IGFBP-2, VEGF-D, GDF-15, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl; and
   one or more reagents for detecting said one or more specific binding members or one or more reagents for detecting and/or quantifying formation of a complex formed by said specific binding member and said biomarker.
304. A diagnostic kit or test device according to statement 303, wherein the specific binding member comprises an antibody molecule or binding fragment thereof.
305. A diagnostic kit or test device according to any of statements 303-304, wherein detecting formation of a complex formed by the specific binding member and the biomarker is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.
306. A diagnostic kit or test device according to any one of statements 301-305, wherein the kit or test device comprises no more than 15, 20, 25, 30, 40, or 50 reagents for detecting.
307. A diagnostic kit or test device according to any one of statements 301-306 for use in a method of predicting a cancer-related outcome in a subject.
308. A diagnostic kit for use, or test device for use, according to statement 307, wherein the cancer-related outcome is susceptibility to treatment with an agent capable of inhibiting or reversing EMT or a chemotherapeutic agent.
309. A diagnostic kit for use, or test device for use, according to statement 308, wherein the agent capable of inhibiting or reversing EMT or chemotherapeutic agent is an Axl inhibitor, Akt3 inhibitor, Slfn11 inhibitor, or PHGDH inhibitor.
310. A diagnostic kit for use, or test device for use, according to statement 308, wherein the agent capable of inhibiting or reversing EMT or chemotherapeutic agent is an Axl inhibitor.
311. A diagnostic kit for use, or test device for use, according to statement 310, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
312. A diagnostic kit for use, or test device for use, according to statement 311, wherein the Axl inhibitor is BGB324/R428/bemcentinib.
313. A diagnostic kit for use, or test device for use, according to statement 311, wherein the Axl inhibitor is an anti-Axl antibody described in WO2015/193428, WO2015/193430, WO2016/097370, or WO2016/166296.
314. A diagnostic kit for use, or test device for use, according to statement 308, wherein the agent capable of inhibiting or reversing EMT or chemotherapeutic agent is an Akt3 inhibitor.
315. A diagnostic kit for use, or test device for use, according to any one of statements 307-310, wherein the subject has, is suspected of having, or has been diagnosed with non-small cell lung cancer (NSCLC).
316. A diagnostic kit for use, or test device for use, according to any one of statements 307-315, wherein the method is a method according to any one of statements 101-247.
317. Use of a reagent for detecting the activity, expression, or amount of a biomarker selected from the group consisting of: NT proBNP, Endostatin, IGFBP-2, VEGF-D, GDF-15, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl, in a method of predicting a cancer-related outcome in a subject.
318. Use of 1, 2, 3, 4, 5, 6, 7, 8, 9, 10 or more reagents for detecting the activity, expression, or amount a biomarker, each of which is selective for a particular biomarker selected from the group consisting of: NT proBNP, Endostatin, IGFBP-2, VEGF-D, GDF-15, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl, in a method of manufacture of a diagnostic kit or test device for use in a method of predicting a cancer-related outcome in a subject.
319. A use according to any one of statements 317-318, wherein the reagent for detecting comprises a specific binding member selective for a particular biomarker.
320. A use according to any one of statement 317-319, wherein the method of predicting a cancer-related outcome in a subject is a method according to any one of statements 101-247.
401. Use of one or more of: NT proBNP, Endostatin, IGFBP-2, VEGF-D, GDF-15, AB-40, ALP, MCP-3, SOD-1, BMP-9, IL-8, MIP-1 beta, RAGE, MPO, CgA, HE4, ADM, B2M, VDBP, Cystatin-C, Hepsin, LDH, and/or Axl, as a biomarker for determining if a subject is susceptible to treatment with an agent capable of inhibiting or reversing EMT or a chemotherapeutic agent.
402. A use according to statement 401, wherein:
   (i) a higher activity, expression, or amount of one or more of said biomarkers in the subject, or sample derived from the subject, compared with a control profile for the respective biomarker is indicative of susceptibility to treatment with an agent capable of inhibiting or reversing EMT or a chemotherapeutic agent; or
   (ii) a lower activity, expression, or amount of one or more of said biomarkers in the subject, or sample derived from the subject, compared with a control profile for the respective biomarker is indicative of susceptibility to treatment with an agent capable of inhibiting or reversing EMT or a chemotherapeutic agent.
403. A use according to statement 401, wherein:
   (i) an increase in the activity, expression, or amount of one or more biomarker relative to a control profile for the respective biomarker after contacting the subject with a cancer therapeutic agent is indicative of increased susceptibility to treatment with an agent capable of inhibiting or reversing EMT or a chemotherapeutic agent; or
   (ii) a decrease in the activity, expression, or amount of one or more biomarker relative to a control profile for the respective biomarker after contacting the subject with a cancer therapeutic agent is indicative of increased susceptibility to treatment with an agent capable of inhibiting or reversing EMT or a chemotherapeutic agent.
404. A use according to any one of statements 401-403, wherein the use is as a biomarker in a method according to any one of statements 101-247
501. A method of treating a subject having, suspected of having, or diagnosed with non-small cell lung cancer (NSCLC), the method comprising:
   identifying a subject susceptible to treatment using a method according to any one of statements 101-247; and
   administering to the subject a therapeutically effective amount of an agent capable of inhibiting or reversing EMT or a chemotherapeutic agent.
502. A method of treating a subject having, suspected of having, or diagnosed with non-small cell lung cancer (NSCLC), the method comprising:
   (a) obtaining a sample from the subject;
   (b) determining if the subject is susceptible to treatment with an agent capable of inhibiting or reversing EMT or a chemotherapeutic agent using a method according to any one of statements 101-247;
   (c) administering to the subject a therapeutically effective amount of an agent capable of inhibiting or reversing EMT or a chemotherapeutic agent.
503. A method of treating a subject having, suspected of having, or diagnosed with non-small cell lung cancer (NSCLC), the method comprising administering a therapeutically effective amount of an agent capable of inhibiting or reversing EMT or chemotherapeutic agent to a subject identified as susceptible to said treatment using a method according to any one of statements 101-247.
504. A method of treating a subject having, suspected of having, or diagnosed with non-small cell lung cancer (NSCLC), the method comprising:
   administering to the subject a therapeutically effective amount of an agent capable of inhibiting or reversing EMT or a chemotherapeutic agent; and
   determining if the subject is susceptible to said treatment using a method according to any one of statements 193-247.
505. A method according to statement 504, wherein the method comprises administering to the subject one or more further therapeutically effective amounts of an agent capable of inhibiting or reversing EMT or a chemotherapeutic agent provided that the subject is identified as susceptible to said treatment.
506. An agent capable of inhibiting or reversing EMT or chemotherapeutic agent for use in a method of treating a subject having, suspected of having, or diagnosed with non-small cell lung cancer (NSCLC),
   wherein the method is a method according to any one of statements 501-505.
507. Use of an agent capable of inhibiting or reversing EMT or chemotherapeutic agent for use in a method of manufacture of a medicament for use in a method of treating a subject having, suspected of having, or diagnosed with non-small cell lung cancer (NSCLC),
   wherein the method of treatment is a method according to any one of statements 501-505.
508. A method, agent for use, or use according to any one of statements 501-507, wherein the agent capable of inhibiting or reversing EMT or chemotherapeutic agent comprises an Axl inhibitor, Akt3 inhibitor, Slfn11 inhibitor, or PHGDH inhibitor.
509. A method, agent for use, or use according to statement 508, wherein the agent capable of inhibiting or reversing EMT or chemotherapeutic agent comprises an Axl inhibitor.
510. A method, agent for use, or use according to statement 509, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
511. A method, agent for use, or use according to statement 510, wherein the Axl inhibitor is BGB324/R428/bemcentinib.
512. A method, agent for use, or use according to statement 510, wherein the Axl inhibitor is an anti-Axl antibody described in WO2015/193428, WO2015/193430, WO2016/097370, or WO2016/166296.
512. A method, agent for use, or use according to statement 508, wherein the agent capable of inhibiting or reversing EMT or chemotherapeutic agent comprises an Akt3 inhibitor.
513. A method, agent for use, or use according to statement 512, wherein the Akt3 inhibitor is an Akt3 inhibitor disclosed in WO2016/102672.
514. A method, agent for use, or use according to any one of statements 501-513, wherein the agent capable of inhibiting or reversing EMT or chemotherapeutic agent is administered as a single agent.
515. A method, agent for use, or use according to any one of statements 501-513, wherein the agent capable of inhibiting or reversing EMT or chemotherapeutic agent is administered in combination with a further cancer treatment.
516. A method, agent for use, or use according to statement 515, wherein the further cancer treatment is selected from the further cancer treatments listed in statement 175.
517. A method, agent for use, or use according to statement 515, wherein the further cancer treatment is:
   (i) a PD-1 inhibitor or PD-L1 inhibitor;
   (ii) a monoclonal antibody such as pembrolizumab, alemtuzumab, bevacizumab, cetuximab, ibritumomab, tiuxetan, or rituximab;
   (iii) a microtubule polymer stabilizer such as the taxanes paclitaxel and docetaxel; and/or
   (iv) a tyrosine kinase inhibitor such as erlotinib hydrochloride, gefitinib, imatinib mesylate, and semaxanib.
518. A method, agent for use, or use according to statement 517, wherein the further cancer treatment is pembrolizumab.
519. A method, agent for use, or use according to statement 517, wherein the further cancer treatment is docetaxel.
520. A method, agent for use, or use according to statement 517, wherein the further cancer treatment is erlotinib.

Facet 7: Lactate Dehydrogenase (LDH)

Melanoma, also known as malignant melanoma, is a class of cancer that develops from melanocytes. These cancers typically occur in the skin, but can also occur in the mouth, intestines, or eye. Chemotherapeutic drug therapies for melanoma include, for example ipilimumab, pembrolizumab, and nivolumab. The overall success rate of such treatments in metastatic melanoma remains limited, and there remains a need for targeted therapies for this cancer type (Hsueh et al, 2016).

Non-small cell lung cancers (NSCLC) are any type of epithelial lung cancer other than small cell lung carcinoma (SCLC), and make up the majority of all lung cancers. Non-small cell lung carcinomas are grouped together because their prognosis and management are similar—the three main histologic classes are: squamous cell lung carcinoma, adenocarcinoma, and large cell lung carcinoma. Adenocarcinoma is the most common histologic type of lung carcinoma. Non-small cell lung cancers are often treated with cisplatin or carboplatin, in combination with gemcitabine, paclitaxel, docetaxel, etoposide or vinorelbine. There remains a need for targeted therapies for NSCLC (Herbst et al, 2018; Zappa et al, 2016).

Accordingly, robust biomarkers for predicting cancer-related outcomes in a subject having melanoma or NSCLC, for example susceptibility to treatment with an agent capable of inhibiting or reversing EMT or a chemotherapeutic agent, would be useful in identifying subjects who are most likely to respond to or benefit from treatment with a particular agent/regimen.

A “responder” subject as described herein is a subject who is likely to, or who does, respond to treatment (or benefit from treatment) with an agent capable of inhibiting or reversing EMT or a chemotherapeutic agent, for example the small molecule Axl kinase inhibitor BGB324/R428/bemcentinib when administered either as a single agent or in combination with another cancer treatment.

Correspondingly, a “non-responder” subject as described herein is a subject who is not likely to, or who does not, respond to treatment (or benefit from treatment) with an agent capable of inhibiting or reversing EMT or a chemotherapeutic agent, for example the small molecule Axl kinase inhibitor BGB324/R428/bemcentinib when administered either as a single agent or in combination with another cancer treatment.

To “respond to treatment” or “benefit from treatment” as used herein means to experience an overall clinical benefit from the treatment. This overall clinical benefit can be any of: prolonged survival, partial or complete disease remission, slowing or absence of disease progression, tumour shrinkage (for example, a reduction in tumour volume of 5, 10, 20, 30, 40% or more), reduction in tumour burden (for example, a reduction in tumour burden of 5, 10, 20, 30, 40% or more), slowing or absence of tumour enlargement, slowing or absence of increase in tumour burden, improved quality of life, progression-free survival, overall survival, or any other positive patient outcome. Suitable methods for determining tumour volume/burden are well known to the skilled person, for example, using: computed tomography (CT), or magnetic resonance imaging (MRI) imaging technologies; X-ray imaging, for example, mammography; ultrasound imaging; nuclear imaging, for example positron emission tomography (PET), PET/CT scans, bone scans, gallium scans, or metaiodobenzylguanidine (MIBG) scans; bioluminescence imaging (BLI); fluorescence imaging (FLI); BD ToF (infrared-based 3D Time-of-Flight camera) imaging.

Biomarkers

The term “marker” or “biomarker” is used herein to refer to a gene or protein whose expression in a subject or sample derived from a subject is altered or modulated, for example, up or down regulated, in cancer. Where the biomarker is a protein, modulation or alteration of expression encompasses modulation through different post-translational modifications.

In accordance with any aspect of this facet of the present disclosure the biomarkers described herein may be defined as follows, wherein:
a Lactate Dehydrogenase (LDH) biomarker comprises an amino acid sequence having at least 70%, 80%, 90%, 95%, 99% or 100% sequence identity with the full-length of the amino acid sequence disclosed at UniProt accession no: P00338-1, P00338-2, P00338-3, P00338-4, or P00338-5 (entry version 224) or a fragment thereof; and/or
a Lactate Dehydrogenase (LDH) biomarker comprises an amino acid sequence having at least 70%, 80%, 90%, 95%, 99% or 100% sequence identity with the full-length of the amino acid sequence disclosed at UniProt accession no: P07195-1 (entry version 218) or a fragment thereof.

The biomarkers described herein may also be defined as a nucleic acid (either DNA or RNA) having at least 70%, 80%, 90%, 95%, 99% or 100% sequence identity with the full-length nucleic acid sequence encoding any of the above amino acid sequences.

Identity may be as defined using sequence comparisons made using FASTA and FASTP (see Pearson & Lipman, 1988. Methods in Enzymology 183: 63-98). Parameters are preferably set, using the default matrix, as follows: Gapopen (penalty for the first residue in a gap): −12 for proteins/-16 for DNA; Gapext (penalty for additional residues in a gap): −2 for proteins/-4 for DNA; KTUP word length: 2 for proteins/6 for DNA.

Pre-Treatment Predictive Biomarkers and Methods

By analysing patient data obtained from clinical trial BGBIL006 (NCT02872259) and BGBC008 (NCT03184571), the authors have discovered that, prior to treatment of subjects having NSCLC or melanoma, the enzyme lactate dehydrogenase (LDH) is present in the serum of “responder” subjects at higher levels than in the serum of “non-responder” subjects—see Example 7. Thus, by assessing the activity, expression, or amount of this biomarker prior to treatment with an agent capable of inhibiting or reversing EMT or a chemotherapeutic agent it is possible to determine the likelihood of that subject being susceptible or responsive to treatment with said agent.

Accordingly, in a first aspect of this facet, the disclosure provides a method of predicting a cancer-related outcome in a subject having, suspected of having, or diagnosed with non-small cell lung cancer (NSCLC) or melanoma, the method comprising assessing the activity, expression, or amount of the biomarker LDH in the subject, or in a sample derived from the subject.

In embodiments in which the subject is a subject having, suspected of having, or diagnosed with non-small cell lung cancer (NSCLC), the method may comprise assessing the activity, expression, or amount of the biomarker LDH and one or more biomarker selected from the group consisting of: Axl, Beta Amyloid 1-40 (AB-40), Aggrecan core protein (Aggrecan), Ferritin (FRTN), Receptor for advanced glycosylation end products (RAGE), Neuronal Cell Adhesion Molecule (Nr-CAM), Sclerostin, Prostate-Specific Antigen, Free (PSA-f), Epidermal Growth Factor Receptor (EGFR), Neurofilament heavy polypeptide (NF-H), Dopamine beta-hydroxylase (DBH), Fibulin-1C (Fib-1C), Myoglobin, Urokinase-type Plasminogen Activator (uPA), Visceral adipose tissue-derived serpin A12 (Vaspin), Interleukin-23 (IL-23), Alpha-2-Macroglobulin (A2Macro), Tissue Inhibitor of Metalloproteinases 2 (TIMP-2), Thyroid-Stimulating Hormone (TSH), Tetranectin, Myeloid Progenitor Inhibitory Factor 1 (MPIF-1), Pancreatic Polypeptide (PPP), Interleukin-7 (IL-7), Pulmonary and Activation-Regulated Chemokine (PARC), Cancer Antigen 15-3 (CA-15-3), Pepsinogen I (PGI), Apolipoprotein E (Apo E), Apolipoprotein C-I (Apo C-I), Macrophage-Stimulating Protein (MSP), C-C motif chemokine 15 (CCL15), Progranulin, Carcinoembryonic antigen-related cell adhesion molecule 1 (CEACAM1), Vitamin D-Binding Protein (VDBP), Carbonic anhydrase 9 (CA-9), and/or Lactate Dehydrogenase (LDH) in the subject, or in a sample derived from the subject.

In embodiments in which the subject is a subject having, suspected of having, or diagnosed with melanoma, the method may comprise assessing the activity, expression, or amount of the biomarker LDH and one or more biomarker selected from the group consisting of: Axl, Interleukin-6 receptor (IL-6r), Chemokine CC-4 (HCC-4), Tissue Inhibitor of Metalloproteinases 1 (TIMP-1), Heparin-Binding EGF-Like Growth Factor (HB-EGF), Antileukoproteinase (ALP), Latency-Associated Peptide of Transforming Growth Factor beta 1 (LAP TGF-b1), Epidermal Growth Factor (EGF), Platelet-Derived Growth Factor BB (PDGF-BB), Osteoprotegerin (OPG), Neutrophil Activating Peptide 2 (NAP-2), ST2, Tumor Necrosis Factor Receptor I (TNF RI), Vascular Endothelial Growth Factor (VEGF), and/or Growth-Regulated alpha protein (GRO-alpha) in the subject, or in a sample derived from the subject.

A “cancer-related outcome” as described herein is a clinical prediction or prognosis associated with cancer. In some embodiments the cancer-related outcome is susceptibility to treatment with an agent capable of inhibiting or reversing EMT or a chemotherapeutic agent.

In some embodiments the method comprises assessing the activity, expression, or amount of LDH in the subject, or in a sample derived from the subject, to obtain a sample profile of LDH; and, making a prediction based on the sample profile.

A “sample profile” as used herein is a numerical value or numerical range corresponding to the activity, expression, or amount of LDH determined in the subject or sample derived from the subject.

In some embodiments of the method the prediction is made by comparing the sample profile to a control profile. A “control profile” as used herein is a numerical value or numerical range corresponding to the activity, expression, or amount of LDH, determined in a control subject or population of control subjects. Suitable control profiles are described in more detail below.

Prior to treatment of a subject, LDH has been found to exhibit up-regulated expression in “responder” subjects—that is, LDH has been found to be present in the serum of “responder” subjects at higher levels than in the serum of “non-responder” subjects.

This difference in expression level between “responder” and “non-responder” subjects can also be expressed in the opposite way. That is to say, prior to treatment of a subject, LDH has been found to exhibit down-regulated expression in “non-responder” subjects—that is, LDH has been found to be present in the serum of “non-responder” subjects at lower levels than in the serum of “responder” subjects.

The discovery of this differential pattern of expression means that, by assessing the activity, expression, or amount of LDH prior to treatment of a subject with an agent, it is possible to determine the likelihood of that subject being susceptible or responsive to treatment with said agent. Accordingly, in particularly preferred embodiments the sample profile is obtained before the subject is contacted with or administered the agent capable of inhibiting or reversing EMT or a chemotherapeutic agent.

A subject who is “susceptible to treatment” with an agent capable of inhibiting or reversing EMT or a chemotherapeutic agent is one who is likely to, or who does, respond to treatment (or benefit from treatment) with an agent capable of inhibiting or reversing EMT or a chemotherapeutic agent. That is, a subject who is “susceptible to treatment” with an agent is a “responder” subject for that agent, as defined above. A prediction that is “indicative of susceptibility” is one which suggests or points towards the likelihood that a subject will respond to or benefit from treatment.

In some embodiments the predictive methods of the disclosure comprise assessing the activity, expression, or amount of LDH in the subject, or in a sample derived from the subject, to obtain a sample profile of LDH; and, making a prediction based on the sample profile. In some embodiments of the method the prediction is made by comparing the sample profile to a control profile, wherein the control profile is a numerical value or numerical range corresponding to the activity, expression, or amount of LDH, determined in a control subject or population of control subjects.

In some embodiments the control profile may be obtained from a population of control subjects having NSCLC and/or a population of control subjects having melanoma. In some embodiments the control profile may be obtained from a population of control subjects not having NSCLC and/or a population of control subjects not having melanoma. In other embodiments the control profile may be obtained from a control subject or population of control subjects having NSCLC and previously found to lack susceptibility to treatment with an agent capable of inhibiting or reversing EMT or a chemotherapeutic agent (that is, non-responder subjects) and/or a control subject or population of control subjects having melanoma and previously found to lack susceptibility to treatment with an agent capable of inhibiting or reversing EMT or a chemotherapeutic agent (that is, non-responder subjects).

In some embodiments the control profile may be a predetermined profile of LDH expression, activity, or amount values, for example a “mean” value, “threshold” value, or “standard range” of values. This predetermined profile of LDH expression, activity, or amount values can be obtained, for example, from a control subject or population of control subjects having NSCLC and/or a control subject or population of control subjects having melanoma, or from a control subject or population of control subjects not having NSCLC and/or a control subject or population of control subjects not having melanoma. This predetermined profile of LDH expression, activity, or amount values could also be obtained, for example, from or a control subject or population of control subjects having NSCLC and previously found to lack susceptibility to treatment with an agent capable of inhibiting or reversing EMT or a chemotherapeutic agent (that is, non-responder subjects) and/or a control subject or population of control subjects having melanoma and previously found to lack susceptibility to treatment with an agent capable of inhibiting or reversing EMT or a chemotherapeutic agent (that is, non-responder subjects).

In some other embodiments the control profile may be obtained from a control subject or population of control subjects having NSCLC and previously found to be susceptible to treatment with an agent capable of inhibiting or reversing EMT or a chemotherapeutic agent (that is, responder subjects) and/or a control subject or population of control subjects having melanoma and previously found to be susceptible to treatment with an agent capable of inhibiting or reversing EMT or a chemotherapeutic agent (that is, responder subjects). In some embodiments the control profile may a predetermined profile of biomarker expression, activity, or amount values, for example a “threshold” value or “standard range” of values. This predetermined profile of biomarker expression, activity, or amount values can be obtained, for example, from a control subject or population of control subjects having NSCLC and previously found to be susceptible to treatment with an agent capable of inhibiting or reversing EMT or a chemotherapeutic agent (that is, responder subjects) and/or a control subject or population of control subjects having melanoma and previously found to be susceptible to treatment with an agent capable of inhibiting or reversing EMT or a chemotherapeutic agent (that is, responder subjects).

In some other embodiments the control profile may be obtained from a control sample having a known amount (for example a “threshold” amount) of LDH. This threshold amount can be obtained, for example, from non-responder or responder subjects as outlined above.

The skilled person is readily able to determine suitable “mean”, “threshold”, or “standard range” values for a particular biomarker.

For example, a “mean” value may be determined by: assessing the activity, expression, or amount of a particular biomarker in a population of subjects having (or not having) NSCLC and/or a control subject or population of control subjects having (or not having) melanoma; and, averaging the biomarker activity, expression, or amount determined to determine a “mean” value.

For example, a “mean” value may be determined by: assessing the activity, expression, or amount of a particular biomarker in a population of subjects; determining which subjects in the population are susceptible to treatment with an agent capable of inhibiting or reversing EMT or a chemotherapeutic agent; and, averaging the biomarker activity, expression, or amount determined in subjects found to be susceptible to treatment with an agent capable of inhibiting or reversing EMT or a chemotherapeutic agent (that is, responder subjects) to determine a “mean” value.

For example, a “threshold” value may be determined by: assessing the activity, expression, or amount of a particular biomarker in a population of subjects; determining which subjects in the population are susceptible to treatment with an agent capable of inhibiting or reversing EMT or a chemotherapeutic agent; and, selecting as appropriate the highest or lowest biomarker activity, expression, or amount determined in a subject found to be susceptible to treatment with an agent capable of inhibiting or reversing EMT or a chemotherapeutic agent (that is, a responder subject) as the “threshold value”.

For example, a “standard range” of values may be determined by: assessing the activity, expression, or amount of a particular biomarker in a population of subjects having (or not having) NSCLC and/or a control subject or population of control subjects having (or not having) melanoma; and, determining a “standard range” of values based on this assessment.

For example, a “standard range” of values may be determined by: assessing the activity, expression, or amount of a particular biomarker in a population of subjects; determining which subjects in the population are susceptible to treatment with an agent capable of inhibiting or reversing EMT or a chemotherapeutic agent; and, determining a “standard range” of values for subjects found to be susceptible to treatment with an agent capable of inhibiting or reversing EMT or a chemotherapeutic agent (that is, a responder subject) based on this assessment.

Where the control profile is a predetermined profile of LDH expression, activity, or amount values, this may be provided as a “look-up table” or data record. Predetermined control profiles of this type may be a numerical value or numerical range corresponding to a measure of central tendency (such as an average, median or mean) amount of LDH determined in a plurality of control subjects (for example non-responder or responder subjects as outlined above). The skilled person is readily able to determine both a measure of central tendency and a measure of variability within a population using standard protein quantification techniques and suitable statistical tools.

Accordingly, in embodiments in which the control profile is: obtained from a population of control subjects having NSCLC and/or a population of control subjects having melanoma; obtained from a population of control subjects not having NSCLC and/or a population of control subjects not having melanoma; obtained from a control subject or population of control subjects having NSCLC and previously found to lack susceptibility to treatment with an agent capable of inhibiting or reversing EMT or a chemotherapeutic agent (that is non-responder subjects) and/or a control subject or population of control subjects having melanoma and previously found to lack susceptibility to treatment with an agent capable of inhibiting or reversing EMT or a chemotherapeutic agent (that is non-responder subjects); a predetermined profile of LDH expression, activity, or amount values (for example a profile of “average, median, or mean” values or “standard ranges” of values) obtained from non-responder subjects; obtained from a control sample having a known “average, median, or mean” value of LDH indicative of non-responder subjects; a predetermined profile of LDH expression, activity, or amount “threshold” values obtained from responder subjects; or, obtained from a control sample having a known “threshold” value of LDH indicative of responder subjects; then, a higher activity, expression, or amount of LDH in a sample profile as compared with the control profile may be indicative of susceptibility to treatment with an agent capable of inhibiting or reversing EMT or a chemotherapeutic agent. Similarly, a lower activity, expression, or amount of LDH in the sample profile as compared with a control profile may be indicative of lack of susceptibility to treatment with an agent capable of inhibiting or reversing EMT or a chemotherapeutic agent.

In embodiments in which the control profile is: obtained from a control subject or population of control subjects having NSCLC and previously found to be susceptible to treatment with an agent capable of inhibiting or reversing EMT or a chemotherapeutic agent (that is, responder subjects); then, if the expression, activity, or amount values in the sample profile are above a “threshold” value typical of a responder subject (that is, a subject having NSCLC and previously found to be susceptible to treatment with an agent capable of inhibiting or reversing EMT or a chemotherapeutic agent) this is indicative of susceptibility to treatment with an agent capable of inhibiting or reversing EMT or a chemotherapeutic agent. Similarly, if the expression, activity, or amount values in the sample profile are below the “threshold” value typical of a responder subject (that is, a subject having NSCLC and previously found to be susceptible to treatment with an agent capable of inhibiting or reversing EMT or a chemotherapeutic agent) this is indicative of a lack of susceptibility to treatment with an agent capable of inhibiting or reversing EMT or a chemotherapeutic agent.

In embodiments in which the control profile is: a predetermined profile of “standard ranges” of LDH expression, activity, or amount values obtained from responder subjects; then, if the expression, activity, or amount values in the sample profile are within the “standard range” of values typical of a responder subject (that is, a subject having NSCLC or melanoma and previously found to be susceptible to treatment with an agent capable of inhibiting or reversing EMT or a chemotherapeutic agent) this is indicative of susceptibility to treatment with an agent capable of inhibiting or reversing EMT or a chemotherapeutic agent. Similarly, if the expression, activity, or amount values in the sample profile are outside the “standard range” of values typical of a responder subject (that is, a subject having NSCLC or melanoma and previously found to be susceptible to treatment with an agent capable of inhibiting or reversing EMT or a chemotherapeutic agent) this is indicative of a lack of susceptibility to treatment with an agent capable of inhibiting or reversing EMT or a chemotherapeutic agent.

In embodiments in which the control profile is: a predetermined profile of “standard ranges” of LDH expression, activity, or amount values obtained from non-responder subjects; then, if the expression, activity, or amount values in the sample profile are within the “standard range” of values typical of a non-responder subject (that is, a subject having NSCLC or melanoma and previously found to lack susceptibility to treatment with an agent capable of inhibiting or reversing EMT or a chemotherapeutic agent) this is indicative of a lack of susceptibility to treatment with an agent capable of inhibiting or reversing EMT or a chemotherapeutic agent. Similarly, if the expression, activity, or amount values in the sample profile are outside the “standard range” of values typical of a non-responder subject (that is, a subject having NSCLC or melanoma and previously found to lack susceptibility to treatment with an agent capable of inhibiting or reversing EMT or a chemotherapeutic agent) this may be indicative of susceptibility to treatment with an agent capable of inhibiting or reversing EMT or a chemotherapeutic agent.

In embodiments in which the control profile is: obtained from a population of control subjects not having NSCLC and/or a control subject or population of control subjects not having melanoma; then, if the expression, activity, or amount values in the sample profile are outside a “standard range” of values typical of subjects not having NSCLC and/or subjects not having melanoma, this is indicative of susceptibility to treatment with an agent capable of inhibiting or reversing EMT or a chemotherapeutic agent. Similarly, if the expression, activity, or amount values in the sample profile are within a “standard range” of values typical of subjects not having NSCLC and/or subjects not having melanoma, this is indicative of a lack of susceptibility to treatment with an agent capable of inhibiting or reversing EMT or a chemotherapeutic agent. In such cases, an expression, activity or amount value may be outside the “standard range” of values when it is either above the upper limit of normal or below the lower limit of normal for a given biomarker. The upper limit of normal and lower limit of normal are the limits of the normal distribution of a given biomarker in a population of control subjects not having NSCLC and/or a control subject or population of control subjects not having melanoma.

In some preferred embodiments, the control profile is a predetermined profile of LDH expression, activity, or amount values. In some such preferred embodiments, the control profile is a “threshold” value determined by: assessing LDH activity, expression, or amount in a population of subjects; determining which subjects in the population are susceptible to treatment with an agent capable of inhibiting or reversing EMT or a chemotherapeutic agent; and, selecting the lowest LDH activity, expression, or amount determined in a subject found to be susceptible to treatment with an agent capable of inhibiting or reversing EMT or a chemotherapeutic agent (that is, a responder subject) as the “threshold” value.

In some such embodiments, the control threshold value of LDH is 287 units per litre (U/L). That is, in some embodiments, an activity of LDH biomarker higher than 287 units per litre (U/L) in a sample profile may be indicative of susceptibility to treatment with an agent capable of inhibiting or reversing EMT or a chemotherapeutic agent.

In other preferred embodiments, the control profile is a “standard range” of values determined by: assessing LDH activity, expression, or amount in a population of subjects not having NSCLC and/or a population of subjects not having melanoma; and, determining a “standard range” of values based on this assessment. In such embodiments, an expression, activity or amount value above or outside the “standard range” of values, for example above the upper limit of normal for LDH, may be indicative of susceptibility to treatment with an agent capable of inhibiting or reversing EMT or a chemotherapeutic agent.

The skilled person is readily able to determine whether the sample profile is indicative of susceptibility or lack of susceptibility to treatment with an agent capable of inhibiting or reversing EMT or a chemotherapeutic agent using suitable statistical tools and comparison with the appropriate control.

In some embodiments the sample profile may be compared with two or more control profiles as described above.

Subject

A “subject” as referred to herein is any species of animal classified as a mammal, and includes but is not restricted to, domestic and farm animals, primates and humans. In some preferred embodiments the subject is a human of any sex or race. In some embodiments, the human is an adult human.

In some embodiments the predictive methods described herein are performed before the subject receives a treatment or course of treatment for NSCLC or melanoma. In other embodiments the predictive method is performed during or after a treatment or course of treatment for NSCLC or melanoma. In some embodiments the treatment or course of treatment for NSCLC or melanoma is an agent capable of inhibiting or reversing EMT. In other embodiments the treatment or course of treatment for NSCLC or melanoma is a chemotherapeutic treatment that is not an agent capable of inhibiting or reversing EMT.

In some embodiments of the predictive methods described herein the subject has previously been treated with an agent capable of inhibiting or reversing EMT or a chemotherapeutic agent. In some embodiments the subject has previously been treated with an agent selected from: a PHGDH inhibitor, a Slfn11 inhibitor, an Axl inhibitor, or an Akt3 inhibitor.

In some embodiments of the predictive methods described herein the subject has previously been treated with an agent that is different to the agent capable of inhibiting or reversing EMT or chemotherapeutic agent for which the subject's response is being predicted. That is, the subject has previously been treated with an agent that is not the agent which the predictive method is determining the subject's susceptibility to. Accordingly, in some embodiments, the subject has not previously been treated with an agent capable of inhibiting or reversing EMT or a chemotherapeutic agent. In some preferred embodiments, the subject has not previously been treated with an Axl inhibitor. In other preferred embodiments, the subject has not previously been treated with an Akt3 inhibitor.

Agents Capable of Inhibiting or Reversing EMT

The present disclosure provides methods for predicting a cancer-related outcome in a subject having, suspected of having, or diagnosed with non-small cell lung cancer (NSCLC) or melanoma. A “cancer-related outcome” is a clinical prediction or prognosis associated with cancer.

The agents capable of inhibiting or reversing EMT for use in this facet of the disclosure are defined as described above in the AML facet (see identically titled section above on page 14, line 19 to page 17, line 2).

In some embodiments in which the subject has, is suspected of having, or has been diagnosed with non-small cell lung cancer (NSCLC) the further cancer treatment is a PD-1 inhibitor or PD-L1 inhibitor. In some such embodiments the further cancer treatment is a monoclonal antibody such as pembrolizumab, alemtuzumab, bevacizumab, cetuximab, ibritumomab, tiuxetan, or rituximab

In some embodiments in which the subject has, is suspected of having, or has been diagnosed with melanoma the further cancer treatment is a PD-1 inhibitor or PD-L1 inhibitor. In some preferred embodiments the further cancer treatment is a monoclonal antibody such as pembrolizumab, alemtuzumab, bevacizumab, cetuximab, ibritumomab tiuxetan, or rituximab, or is a B-Raf inhibitor such as debrafenib, a MEK inhibitor such as trametinib or a combination therapy of these agents. In some particularly preferred embodiments the further cancer treatment is pembrolizumab. In other particularly preferred embodiments the further cancer treatment is a combination therapy of debrafenib and trametinib.

Assessing Biomarker Activity, Expression, Amount

In some embodiments of the predictive methods described herein, assessing the activity, expression, or amount of LDH in a subject, or in a sample derived from a subject, comprises: contacting a sample, or an extract from a sample, with a reagent for detecting the activity, expression, or amount of LDH, which reagent is selective for LDH; and, detecting and/or quantifying said reagents for detecting.

In some preferred embodiments, assessing the activity of LDH comprises determining the enzymatic activity of LDH in the subject or sample derived from the subject. Preferably the enzymatic activity is assessed by determining the level or amount of a substrate or product of the enzymatic reaction catalysed by LDH in the subject or sample derived from the subject. In particularly preferred embodiments the enzymatic activity is assessed by determining the level or amount of reduced nicotinamide adenine dinucleotide (NADH) in the subject or sample derived from the subject. In some embodiments this may be determined using a colorimetric assay, for example, the colorimetric assay available from Sigma-Aldrich with catalogue number MAK066. Other suitable assays for assessing activity of LDH are well known to the skilled person. In particularly preferred embodiments the activity of LDH is assessed by determining the enzymatic activity of LDH in blood or serum.

In some embodiments, assessing the expression or amount of LDH comprises determining the level of LDH protein or mRNA in the subject or sample derived from the subject. The expression or amount of LDH may be assessed by determining the level of protein expression. The expression or amount of LDH may be assessed by determining the level of protein expression in serum.

The term “expression” refers to the transcription of a gene's DNA template to produce the corresponding mRNA and translation of this mRNA to produce the corresponding gene product (i.e., a peptide, polypeptide, or protein) as well as the “expression” of a protein in one or more forms that may have been modified post translation.

In some embodiments determining the level of LDH protein or mRNA comprises: contacting a sample, or an extract from a sample, with a specific binding members, which specific binding member selectively binds to LDH protein or mRNA; and, detecting and/or quantifying formation of complex formed by said specific binding member and said LDH protein or mRNA. In some embodiments the specific binding member may comprise an antibody molecule or a binding fragment thereof.

A range of suitable techniques for determining protein and mRNA expression levels are well known in the art, for example microarray analysis, Western blotting, and PCR techniques such as QPCR. In some embodiments, determining the expression or amount of the one or more biomarker protein or mRNA may comprise use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.

In some embodiments, the expression or amount of LDH is determined as an absolute amount of protein or mRNA in the sample. In such cases, the protein or mRNA may be quantified in the sample and the amount expressed as a measure of the sample, for example the volume, mass, or total protein content of the sample.

In some embodiments, the expression or amount of LDH is determined as a relative amount of protein or mRNA in the sample, wherein the relative amount is determined relative to a reference protein or mRNA in the sample. Determining the relative amount of LDH protein or mRNA in this way allows for normalisation to, e.g., account for differences in total protein concentration and to remove bias from sample to sample.

Preferably the reference protein or mRNA is one whose expression or amount does not vary significantly between subjects having cancer and subjects not having cancer, in particular NSCLC or melanoma. That is, the reference protein or mRNA is preferably one whose expression or amount is not altered by NSCLC or melanoma. Accordingly, the reference protein or mRNA may comprise one or more protein or mRNA which is not one of the biomarkers described herein. When expressing the expression or amount of LDH as a relative amount, the absolute amount of LDH protein or mRNA in the sample may be divided by the absolute amount of the reference protein or mRNA in the sample.

Assessing Biomarker Expression at the RNA Level

Gene expression can be detected at the RNA level. RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland). Typical assay formats utilising ribonucleic acid hybridisation include nuclear run-on assays, RT-PCR, RNase protection assays (Melton et al., Nuc. Acids Res. 12:7035), Northern blotting and In Situ hybridization. Gene expression can also be detected by microarray analysis as described below.

Assessing Biomarker Expression at the Protein Level

LDH level may be assessed by measuring protein expression. Altered gene or protein expression may also be detected by measuring the polypeptides encoded by the gene. This may be achieved by using molecules which bind to the polypeptides encoded by the biomarker gene. Suitable molecules/agents which bind either directly or indirectly to the polypeptides in order to detect the presence of the protein include naturally occurring molecules such as peptides and proteins, for example antibodies, or they may be synthetic molecules.

Antibodies may be derived from commercial sources or through techniques which are familiar to those skilled in the art. In one embodiment, and where altered expression manifests itself through the expression of alteration of post translationally-modified forms of a protein biomarker, antibodies specific for those different forms may be used. For the purposes of this disclosure, the term “antibody”, unless specified to the contrary, includes whole antibodies, or fragments of whole antibodies which retain their binding activity for a target antigen. Such fragments include Fv, F(ab′) and F(ab′)2 fragments, as well as single chain antibodies (scFv). Furthermore, the antibodies and fragments thereof may be humanised antibodies, for example as described in EP239400A. For example: monoclonal and polyclonal antibodies, recombinant antibodies, proteolytic and recombinant fragments of antibodies (Fab, Fv, scFv, diabodies), single-domain antibodies (VHH, sdAb, nanobodies, IgNAR, VNAR), and proteins unrelated to antibodies, which have been engineered to have antibody-like specific binding. Antibodies may be bound to a solid support and/or packaged into kits in a suitable container along with suitable reagents, controls, instructions and the like.

Arrays

Array technology and the various techniques and applications associated with it are well known in the art. Array technology overcomes the disadvantages with traditional methods in molecular biology, which generally work on a “one gene in one experiment” basis, resulting in low throughput and the inability to appreciate the “whole picture” of gene function. In the context of the products and methods of the present disclosure, array technology can be used, for example, in the analysis of the expression of biomarker proteins or mRNA.

In general, any library or group of samples may be arranged in an orderly manner into an array, by spatially separating the members of the library or group. Examples of suitable libraries for arraying include nucleic acid libraries (including DNA, cDNA, oligonucleotide, etc. libraries), peptide, polypeptide and protein libraries, as well as libraries comprising any molecules, such as ligand libraries, among others. Accordingly, where reference is made to a “library” in this document, unless the context dictates otherwise, such reference should be taken to include reference to a library in the form of an array.

Proteins, polypeptides, etc., may also be immobilised in arrays. For example, antibodies have been used in microarray analysis of the proteome using protein chips (Borrebaeck C A, 2000, Immunol Today 21(8):379-82). Polypeptide arrays are reviewed in, for example, MacBeath and Schreiber, 2000, Science, 289(5485):1760-1763.

Sample

Suitable samples include, but are not limited to, tissue samples such as tissue biopsy, blood, urine, buccal scrapes etc., as well as serum, plasma, or tissue culture supernatant samples. In preferred embodiments the expression or amount of LDH is assessed by determining the level of protein expression in the sample. In some preferred embodiments the sample is a blood, serum, or plasma sample. In some particularly preferred embodiments the sample is a serum sample.

When assessing the expression or amount of LDH protein or mRNA in serum or plasma samples derived from a subject, samples are removed and subjected to analytical techniques such as flow cytometry, mass cytometry (CyTOF), ELISA, PET, and SELDI-TOF MS. In some embodiments, the method may comprise extracting RNA from said sample and detecting gene expression by QPCR. In some other embodiments, gene expression may be detected by detecting protein products by, for example, Western Blot.

In some embodiments, the level of expression of LDH is assessed by determining the copy number of the gene encoding the one or more biomarker. Copy number (i.e. gene duplication events) may be determined using standard techniques known in the art, e.g. using a DNA chip as described in Jiang et al. (Jiang Q, Ho Y Y, Hao L, Nichols Berrios C, Chakravarti A. Copy number variants in candidate genes are genetic modifiers of Hirschsprung disease. PLoS One. 2011; 6(6)).

Is some preferred embodiments of the predictive methods described herein, the method is performed in vitro or ex vivo.

Methods of Selecting a Subject for Treatment

A second aspect of this facet of the present disclosure relates to methods of selecting subjects having, suspected of having, or diagnosed with non-small cell lung cancer (NSCLC) or melanoma for treatment with an agent capable of inhibiting or reversing EMT or a chemotherapeutic agent. In some embodiments the method comprises: identifying subjects susceptible to treatment with an agent capable of inhibiting or reversing EMT or a chemotherapeutic agent using a predictive method defined in accordance with the above described first aspect of this facet of the disclosure; and, selecting thus identified subjects for treatment.

In some embodiments this aspect relates to methods of selecting subjects having, suspected of having, or diagnosed with non-small cell lung cancer (NSCLC) or melanoma for continued treatment with an agent capable of inhibiting or reversing EMT or a chemotherapeutic agent.

In some embodiments of this aspect of the disclosure, the treatment comprises administering to the subject a therapeutically effective amount of an agent capable of inhibiting or reversing EMT or a chemotherapeutic agent as described herein. Accordingly, in some embodiments the treatment comprises an Axl inhibitor, Akt3 inhibitor, Slfn11 inhibitor, or PHGDH inhibitor. In some preferred embodiments the treatment comprises an Axl inhibitor, for example, BGB324/R428/bemcentinib, cabozantinib, TP-0903, foretinib, merestinib, bosutinib, gilteritinib, crizotinib, amuvatinib, sunitinib, sitravatinib, LDC1267, or UNC2025. Other Axl inhibitors include the anti-Axl antibodies described in WO2015/193428, WO2015/193430, WO2016/097370, and WO2016/166296. In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In some other preferred embodiments, the treatment comprises an Akt3 inhibitor. In some embodiments the Akt3 inhibitor is an Akt3 inhibitor disclosed in WO2016/102672.

In some embodiments of this aspect of the disclosure, the treatment is administered as a single agent. In other embodiments, the treatment is administered in combination with a further cancer treatment. Suitable further cancer treatments are outlined in detail above.

In some embodiments in which the subject has, is suspected of having, or has been diagnosed with non-small cell lung cancer (NSCLC) the further cancer treatment is a PD-1 inhibitor or PD-L1 inhibitor. In some such embodiments the further cancer treatment is a monoclonal antibody such as pembrolizumab, alemtuzumab, bevacizumab, cetuximab, ibritumomab, tiuxetan, or rituximab

In some embodiments in which the subject has, is suspected of having, or has been diagnosed with melanoma the further cancer treatment is a PD-1 inhibitor or PD-L1 inhibitor. In some preferred embodiments the further cancer treatment is a monoclonal antibody such as pembrolizumab, alemtuzumab, bevacizumab, cetuximab, ibritumomab tiuxetan, or rituximab, or is a B-Raf inhibitor such as debrafenib, a MEK inhibitor such as trametinib or a combination therapy of these agents. In some particularly preferred embodiments the further cancer treatment is pembrolizumab. In other particularly preferred embodiments the further cancer treatment is a combination therapy of debrafenib and trametinib.

Kits

A third aspect of this facet of the present disclosure relates to diagnostic kits and test devices. In some embodiments the disclosure provides a diagnostic kit comprising a reagent for detecting the activity, expression, or amount of LDH, which reagent for detecting is selective for LDH.

In other embodiments the disclosure provides a test device comprising a reagent for detecting the activity, expression, or amount of LDH, which reagent for detecting is selective for LDH.

In some embodiments the diagnostic kit or test device comprises a reagent for determining the enzymatic activity of LDH. In some embodiments the diagnostic kit or test device comprises a reagent for determining the level or amount of a substrate or product of the enzymatic reaction catalysed by LDH. In some embodiments the diagnostic kit or test device comprises a reagent for determining the level or amount of reduced nicotinamide adenine dinucleotide (NADH). In some embodiments the diagnostic kit or test device comprises a probe specific for NADH. In some embodiments the probe interacts with NADH to produce a signal, for example a colour.

In some embodiments the diagnostic kit or test device comprises a specific binding member which selectively binds to LDH; and, one or more reagents for detecting said specific binding members, or one or more reagents for detecting and/or quantifying formation of a complex formed by said specific binding member and LDH.

In some embodiments of this aspect of the disclosure the diagnostic kit or test device comprises a plurality of said specific binding members provided in the form of an array on a substrate or conjugated to a plurality of particles, such as beads or microspheres. The particles may be encoded with a detectable label. In some embodiments the specific binding member comprises an antibody molecule or a binding fragment thereof.

In some embodiments of the diagnostic kit or test device detecting formation of a complex formed by a specific binding member and LDH is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.

In some embodiments of the diagnostic kit or test device, the kit or test device comprises no more than 2, 5, 10, 15, 20, 25, 30, 40, or 50 reagents for detecting. In some embodiments of the diagnostic kit or test device, the kit or test device comprises no more than 2, 5, 10, 15, 20, 25, 30, 40, or 50 specific binding members.

The present disclosure also provides use of such diagnostic kits and test devices in methods of predicting a cancer-related outcome in a subject. In preferred embodiments the cancer-related outcome is susceptibility to treatment with an agent capable of inhibiting or reversing EMT or a chemotherapeutic agent as described herein.

In some embodiments the agent capable of inhibiting or reversing EMT or chemotherapeutic agent is an Axl inhibitor, Akt3 inhibitor, Slfn11 inhibitor, or PHGDH inhibitor. In some preferred embodiments the agent capable of inhibiting or reversing EMT or chemotherapeutic agent is an Axl inhibitor, for example BGB324/R428/bemcentinib, cabozantinib, TP-0903, foretinib, merestinib, bosutinib, gilteritinib, crizotinib, amuvatinib, sunitinib, sitravatinib, LDC1267, or UNC2025. Other Axl inhibitors include the anti-Axl antibodies described in WO2015/193428, WO2015/193430, WO2016/097370, and WO2016/166296. In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In some embodiments of the diagnostic kit or test device for use in a predictive method, the subject has, is suspected of having, or has been diagnosed with NSCLC or melanoma. In some embodiments of the diagnostic kit or test device for use in a predictive method, the subject has, is suspected of having, or has been diagnosed with NSCLC. In some embodiments of the diagnostic kit or test device for use in a predictive method, the subject has, is suspected of having, or has been diagnosed with melanoma.

In some embodiments the method may be as defined in accordance with the above described first aspect of this facet of the disclosure.

A fourth aspect of this facet of the present disclosure relates to the use of a reagent for detecting the activity, expression, or amount of LDH, in a method of predicting a cancer-related outcome in a subject.

A fifth aspect of this facet of the disclosure relates to the use of a reagent for detecting the activity, expression, or amount of LDH, which reagent for detecting is selective for LDH, in a method of manufacture of a diagnostic kit or test device for use in a method of predicting a cancer-related outcome in a subject. In some embodiments of these uses the reagent for detecting comprises a specific binding member selective for a particular biomarker. The specific binding member may comprise an antibody molecule or a binding fragment thereof.

In some embodiments the method may be as defined in accordance with the above described first aspect of this facet of the disclosure.

Uses

A sixth aspect of this facet of the present disclosure relates to the use of LDH as a biomarker for determining if a subject is susceptible to treatment with an agent capable of inhibiting or reversing EMT or a chemotherapeutic agent.

In some embodiments the use relates to use of LDH, and at least 1, 2, 3, 4, 5, 6, 7, 8, 9, 10, or more of: Axl, AB-40, Aggrecan, FRTN, RAGE, Nr-CAM, Sclerostin, PSA-f, EGFR, NF-H, DBH, Fib-1C, Myoglobin, uPA, Vaspin, IL-23, A2Macro, TIMP-2, TSH, Tetranectin, MPIF-1, PPP, IL-7, PARC, CA-15-3, PGI, Apo E, Apo C-I, MSP, CCL15, Progranulin, CEACAM1, VDBP, and/or CA-9 as a biomarker for determining if a subject is susceptible to treatment with an agent capable of inhibiting or reversing EMT or a chemotherapeutic agent.

In some embodiments the use relates to use of LDH, and at least 1, 2, 3, 4, 5, 6, 7, 8, 9, 10, or more of: Axl, IL-6r, HCC-4, TIMP-1, HB-EGF, ALP, LAP TGF-b1, EGF, PDGF-BB, OPG, NAP-2, ST2, TNF RI, VEGF, and/or GRO-alpha as a biomarker for determining if a subject is susceptible to treatment with an agent capable of inhibiting or reversing EMT or a chemotherapeutic agent.

In some embodiments the use is as a biomarker in a method as defined in accordance with the above described first aspect of this facet of the disclosure.

Methods of Treating a Subject

A seventh aspect of this facet of the present disclosure relates to methods of treating a subject having, suspected of having, or diagnosed with non-small cell lung cancer (NSCLC) or melanoma. In some embodiments the method comprises: identifying a subject for treatment using a predictive method defined in accordance with the above described first aspect of this facet of the disclosure; and, administering to the subject a therapeutically effective amount of an agent capable of inhibiting or reversing EMT or a chemotherapeutic agent.

Accordingly, in some embodiments, the method comprises: (a) obtaining a sample from the subject; (b) determining if the subject is susceptible to treatment with an agent capable of inhibiting or reversing EMT or a chemotherapeutic agent using a predictive method defined in accordance with the above described first aspect of this facet of the disclosure; and, (c) administering to the subject a therapeutically effective amount of an agent capable of inhibiting or reversing EMT or a chemotherapeutic agent.

In some embodiments the method comprises administering a therapeutically effective amount of an agent capable of inhibiting or reversing EMT or chemotherapeutic agent to a subject identified as susceptible to said treatment using a predictive method defined in accordance with the above described first aspect of this facet of the disclosure.

An eighth aspect of this facet of the present disclosure relates to an agent capable of inhibiting or reversing EMT or chemotherapeutic agent for use in a method of treating a subject having, suspected of having, or diagnosed with non-small cell lung cancer (NSCLC) or melanoma. In some embodiments the method of treatment is a method as defined above.

A ninth aspect of this facet of the present disclosure relates to use of an agent capable of inhibiting or reversing EMT or chemotherapeutic agent for use in a method of manufacture of a medicament for use in a method of treating a subject having, suspected of having, or diagnosed with non-small cell lung cancer (NSCLC) or melanoma. In some embodiments the method of treatment is a method as defined above.

In some embodiments of these methods, agents for use, or uses, the agent capable of inhibiting or reversing EMT or chemotherapeutic agent comprises an Axl inhibitor, Akt3 inhibitor, Slfn11 inhibitor, or PHGDH inhibitor. In some preferred embodiments the agent capable of inhibiting or reversing EMT or chemotherapeutic agent is an Axl inhibitor, for example BGB324/R428/bemcentinib, cabozantinib, TP-0903, foretinib, merestinib, bosutinib, gilteritinib, crizotinib, amuvatinib, sunitinib, sitravatinib, LDC1267, or UNC2025. Other Axl inhibitors include the anti-Axl antibodies described in WO2015/193428, WO2015/193430, WO2016/097370, and WO2016/166296. In some preferred embodiments the Axl inhibitor is an anti-Axl antibody described in WO2015/193428, WO2015/193430, WO2016/097370, or WO2016/166296. In some particularly preferred embodiments the Axl inhibitor is BGB324/R428/bemcentinib.

In some other preferred embodiments, the treatment comprises an Akt3 inhibitor. In some embodiments the Akt3 inhibitor is an Akt3 inhibitor disclosed in WO2016/102672.

In some embodiments of these methods, agents for use, or uses, the treatment is administered as a single agent. In other embodiments, the treatment is administered in combination with a further cancer treatment. Suitable further cancer treatments are outlined in detail above.

In some preferred embodiments the further cancer treatment is a PD-1 inhibitor or PD-L1 inhibitor. In some preferred embodiments the further cancer treatment is a monoclonal antibody such as pembrolizumab, alemtuzumab, bevacizumab, cetuximab, ibritumomab tiuxetan, or rituximab, or is a B-Raf inhibitor such as debrafenib, a MEK inhibitor such as trametinib or a combination therapy of these agents. In some particularly preferred embodiments the further cancer treatment is pembrolizumab. In other particularly preferred embodiments the further cancer treatment is a combination therapy of debrafenib and trametinib.

Statements of Disclosure

The following numbered statements relate to aspects of this facet of the present disclosure, and form part of the description:
1. A method of predicting a cancer-related outcome in a subject, the method comprising assessing the activity, expression, or amount of an LDH biomarker in the subject, or in a sample derived from the subject;
   wherein the subject has, is suspected of having, or has been diagnosed with non-small cell lung cancer (NSCLC) or melanoma.
2. A method according to statement 1, wherein the method comprises:
   assessing the activity, expression, or amount of one an LDH biomarker in the subject, or in a sample derived from the subject, to obtain a sample profile of the LDH biomarker; and
   making a prediction based on the sample profile of the LDH biomarker;
   optionally wherein the prediction is made by comparing the sample profile to a control profile.
3. A method according to statement 2, wherein the sample profile is obtained before the subject is contacted with or administered an agent capable of inhibiting or reversing EMT or a chemotherapeutic agent.
4. A method according to statement 3, wherein the control profile is:
   (i) obtained from a population of control subjects having non-small cell lung cancer (NSCLC) or melanoma; or
   (ii) obtained from a population of control subjects not having non-small cell lung cancer (NSCLC) or melanoma; or
   (iii) obtained from a control subject or population of control subjects having non-small cell lung cancer (NSCLC) or melanoma and previously found to lack susceptibility to treatment with an agent capable of inhibiting or reversing EMT or a chemotherapeutic agent;
   (iv) a predetermined profile of “average, median, or mean” or “standard ranges” of biomarker expression, activity, or amount values obtained from a control subject or population of control subjects having non-small cell lung cancer (NSCLC) or melanoma and previously found to lack susceptibility to treatment with an agent capable of inhibiting or reversing EMT or a chemotherapeutic agent;
   (v) obtained from a control sample having a known “average, median, or mean” value of biomarkers indicative of a control subject or population of control subjects having non-small cell lung cancer (NSCLC) or melanoma and previously found to lack susceptibility to treatment with an agent capable of inhibiting or reversing EMT or a chemotherapeutic agent;
   (vi) a predetermined profile of biomarker expression, activity, or amount “threshold” values obtained from a control subject or population of control subjects having non-small cell lung cancer (NSCLC) or melanoma and previously found to be susceptible to treatment with an agent capable of inhibiting or reversing EMT or a chemotherapeutic agent; or
   (vii) obtained from a control sample having a known “threshold” value of biomarkers indicative of a control subject or population of control subjects having non-small cell lung cancer (NSCLC) or melanoma and previously found to be susceptible to treatment with an agent capable of inhibiting or reversing EMT or a chemotherapeutic agent.
5. A method according to statement 4, wherein the control profile is:
   (i) a “threshold” value determined by: assessing LDH activity, expression, or amount in a population of subjects; determining which subjects in the population are susceptible to treatment with an agent capable of inhibiting or reversing EMT or a chemotherapeutic agent; and, selecting the lowest LDH activity, expression, or amount determined in a subject found to be susceptible to treatment with an agent capable of inhibiting or reversing EMT or a chemotherapeutic agent (that is, a responder subject) as the “threshold” value; or
   (ii) a “standard range” of values determined by: assessing LDH activity, expression, or amount in a population of subjects not having NSCLC and/or a population of subjects not having melanoma; and, determining a “standard range” of LDH activity, expression, or amount values in these subjects.
6. A method according to any one of statements 3-5, wherein a higher activity, expression, or amount of LDH biomarker in the sample profile as compared with the control profile is indicative of susceptibility to treatment with an agent capable of inhibiting or reversing EMT or a chemotherapeutic agent.
7. A method according to any one of statements 1-6, wherein the method comprises assessing the activity, expression, or amount of one or more further biomarker selected from the group consisting of:
   (i) Axl, Beta Amyloid 1-40 (AB-40), Aggrecan core protein (Aggrecan), Ferritin (FRTN), Receptor for advanced glycosylation end products (RAGE), Neuronal Cell Adhesion Molecule (Nr-CAM), Sclerostin, Prostate-Specific Antigen, Free (PSA-f), Epidermal Growth Factor Receptor (EGFR), Neurofilament heavy polypeptide (NF-H), Dopamine beta-hydroxylase (DBH), Fibulin-1C (Fib-1C), Myoglobin, Urokinase-type Plasminogen Activator (uPA), Visceral adipose tissue-derived serpin A12 (Vaspin), Interleukin-23 (IL-23), Alpha-2-Macroglobulin (A2Macro), Tissue Inhibitor of Metalloproteinases 2 (TIMP-2), Thyroid-Stimulating Hormone (TSH), Tetranectin, Myeloid Progenitor Inhibitory Factor 1 (MPIF-1), Pancreatic Polypeptide (PPP), Interleukin-7 (IL-7), Pulmonary and Activation-Regulated Chemokine (PARC), Cancer Antigen 15-3 (CA-15-3), Pepsinogen I (PGI), Apolipoprotein E (Apo E), Apolipoprotein C-I (Apo C-I), Macrophage-Stimulating Protein (MSP), C-C motif chemokine 15 (CCL15), Progranulin, Carcinoembryonic antigen-related cell adhesion molecule 1 (CEACAM1), Vitamin D-Binding Protein (VDBP), Carbonic anhydrase 9 (CA-9), and/or Lactate Dehydrogenase (LDH); or
   (ii) Axl, Interleukin-6 receptor (IL-6r), Chemokine CC-4 (HCC-4), Tissue Inhibitor of Metalloproteinases 1 (TIMP-1), Heparin-Binding EGF-Like Growth Factor (HB-EGF), Antileukoproteinase (ALP), Latency-Associated Peptide of Transforming Growth Factor beta 1 (LAP TGF-b1), Epidermal Growth Factor (EGF), Platelet-Derived Growth Factor BB (PDGF-BB), Osteoprotegerin (OPG), Neutrophil Activating Peptide 2 (NAP-2), ST2, Tumor Necrosis Factor Receptor I (TNF RI), Vascular Endothelial Growth Factor (VEGF), and/or Growth-Regulated alpha protein (GRO-alpha);
   in the subject, or in a sample derived from the subject.
8. A method according to any one of statements 1-7, wherein assessing the activity, expression, or amount of LDH biomarker in the subject, or in a sample derived from the subject, comprises:
   contacting a sample, or an extract from the sample, with a reagent for detecting the activity, expression, or amount of LDH biomarker, which reagent is selective for the LDH biomarker; and
   detecting and/or quantifying said reagent for detecting.
9. A method according to statement 8, wherein assessing the activity of LDH biomarker in the subject, or in a sample derived from the subject, comprises determining the enzymatic activity of LDH in the subject or in a sample derived from the subject;

optionally wherein the enzymatic activity is assessed by determining the level or amount of a substrate or product of the enzymatic reaction catalysed by LDH
10. A method according to statement 8, wherein assessing the expression or amount of LDH biomarker in the subject, or in a sample derived from the subject, comprises:
   contacting a sample, or an extract from the sample, with a specific binding member, which specific binding member selectively binds to LDH protein or mRNA; and
   detecting and/or quantifying formation of a complex formed by said specific binding member and said LDH protein or mRNA;
   optionally wherein the specific binding member comprises an antibody molecule or binding fragment thereof.
11. A method according to any one of statements 3-10, wherein the agent capable of inhibiting or reversing EMT or chemotherapeutic agent is an Axl inhibitor.
12. A method according to statement 11, wherein the Axl inhibitor is BGB324/R428/bemcentinib.
13. A method according to statement 11, wherein the Axl inhibitor is an anti-Axl antibody described in WO2015/193428, WO2015/193430, WO2016/097370, or WO2016/166296
14. A method according to any one of statements 3-13, wherein the agent capable of inhibiting or reversing EMT is administered in combination with a further cancer treatment.
15. A method according to statement 14, wherein the further cancer treatment is a monoclonal antibody such as pembrolizumab, alemtuzumab, bevacizumab, cetuximab, ibritumomab tiuxetan, or rituximab, or is a B-Raf inhibitor such as debrafenib, a MEK inhibitor such as trametinib or a combination therapy of these agents.
16. A method according to statement 15, wherein the further cancer treatment is pembrolizumab.
17. A method according to statement 15, wherein the further cancer treatment is a combination therapy of debrafenib and trametinib.
18. A method according to any one of statements 1-17, wherein the subject is mammalian; optionally wherein the subject is human.
19. A method according to any one of statements 1-18, wherein the sample is a blood, serum, or plasma sample;

optionally wherein the sample is: (a) a serum sample; or (b) a blood sample.
20. A method according to any one of statements 1-19, wherein the method is performed in vitro or ex vivo.
21. A method of selecting subjects having, suspected of having, or diagnosed with non-small cell lung cancer (NSCLC) or melanoma for treatment with an agent capable of inhibiting or reversing EMT or a chemotherapeutic agent, the method comprising:
   identifying subjects susceptible to treatment with an agent capable of inhibiting or reversing EMT or a chemotherapeutic agent using a method according to any one of statements 1-20; and
   selecting thus identified subjects for treatment.
22. A method according to statement 21 wherein the agent capable of inhibiting or reversing EMT or chemotherapeutic agent is an Axl inhibitor;

optionally wherein the agent capable of inhibiting or reversing EMT is administered in combination with a further cancer treatment.
23. A diagnostic kit or test device comprising a reagent for detecting the activity, expression, or amount of an LDH biomarker, which reagent for detecting is selective for LDH.
24. A diagnostic kit or test device according to statement 23 for use in a method of predicting a cancer-related outcome in a subject,
   optionally wherein the method is a method according to any one of statements 1-20.
25. Use of LDH as a biomarker for determining if a subject is susceptible to treatment with an agent capable of inhibiting or reversing EMT or a chemotherapeutic agent,
   optionally wherein the use is as a biomarker in a method according to any one of statements 1-20.
101. A method of predicting a cancer-related outcome in a subject, the method comprising assessing the activity, expression, or amount of an LDH biomarker in the subject, or in a sample derived from the subject;
   wherein the subject has, is suspected of having, or has been diagnosed with non-small cell lung cancer (NSCLC) or melanoma.
102. A method according to statement 101, wherein the method comprises:
   assessing the activity, expression, or amount of an LDH biomarker in the subject, or in a sample derived from the subject, to obtain a sample profile of the LDH biomarker; and
   making a prediction based on the sample profile of the LDH biomarker.
103. A method according to statement 101 or 102, wherein the method comprises assessing the activity, expression, or amount of one or more further biomarker selected from the group consisting of:
   (i) Axl, Beta Amyloid 1-40 (AB-40), Aggrecan core protein (Aggrecan), Ferritin (FRTN), Receptor for advanced glycosylation end products (RAGE), Neuronal Cell Adhesion Molecule (Nr-CAM), Sclerostin, Prostate-Specific Antigen, Free (PSA-f), Epidermal Growth Factor Receptor (EGFR), Neurofilament heavy polypeptide (NF-H), Dopamine beta-hydroxylase (DBH), Fibulin-1C (Fib-1C), Myoglobin, Urokinase-type Plasminogen Activator (uPA), Visceral adipose tissue-derived serpin A12 (Vaspin), Interleukin-23 (IL-23), Alpha-2-Macroglobulin (A2Macro), Tissue Inhibitor of Metalloproteinases 2 (TIMP-2), Thyroid-Stimulating Hormone (TSH), Tetranectin, Myeloid Progenitor Inhibitory Factor 1 (MPIF-1), Pancreatic Polypeptide (PPP), Interleukin-7 (IL-7), Pulmonary and Activation-Regulated Chemokine (PARC), Cancer Antigen 15-3 (CA-15-3), Pepsinogen I (PGI), Apolipoprotein E (Apo E), Apolipoprotein C-I (Apo C-I), Macrophage-Stimulating Protein (MSP), C-C motif chemokine 15 (CCL15), Progranulin, Carcinoembryonic antigen-related cell adhesion molecule 1 (CEACAM1), Vitamin D-Binding Protein (VDBP), Carbonic anhydrase 9 (CA-9), and/or Lactate Dehydrogenase (LDH); or
   (ii) Axl, Interleukin-6 receptor (IL-6r), Chemokine CC-4 (HCC-4), Tissue Inhibitor of Metalloproteinases 1 (TIMP-1), Heparin-Binding EGF-Like Growth Factor (HB-EGF), Antileukoproteinase (ALP), Latency-Associated Peptide of Transforming Growth Factor beta 1 (LAP TGF-b1), Epidermal Growth Factor (EGF), Platelet-Derived Growth Factor BB (PDGF-BB), Osteoprotegerin (OPG), Neutrophil Activating Peptide 2 (NAP-2), ST2, Tumor Necrosis Factor Receptor I (TNF RI), Vascular Endothelial Growth Factor (VEGF), and/or Growth-Regulated alpha protein (GRO-alpha);
   in the subject, or in a sample derived from the subject.
104. A method according to statement 102 or 103, wherein the prediction is made by comparing the sample profile to a control profile.
105. A method according to statement 104, wherein the sample profile is obtained before the subject is contacted with or administered an agent capable of inhibiting or reversing EMT or a chemotherapeutic agent.
106. A method according to statement 105, wherein the control profile is:
   (i) obtained from a population of control subjects having non-small cell lung cancer (NSCLC) or melanoma; or
   (ii) obtained from a population of control subjects not having non-small cell lung cancer (NSCLC) or melanoma
   (iii) obtained from a control subject or population of control subjects having non-small cell lung cancer (NSCLC) or melanoma and previously found to lack susceptibility to treatment with an agent capable of inhibiting or reversing EMT or a chemotherapeutic agent (that is, non-responder subjects);
   (iv) a predetermined profile of LDH biomarker expression, activity, or amount values, for example a profile of “average, median, or mean” values or “standard ranges” of values, obtained from a control subject or population of control subjects having non-small cell lung cancer (NSCLC) or melanoma and previously found to lack susceptibility to treatment with an agent capable of inhibiting or reversing EMT or a chemotherapeutic agent (that is, non-responder subjects);
   (v) obtained from a control sample having a known “average, median, or mean” value of LDH biomarker indicative of a control subject or population of control subjects having non-small cell lung cancer (NSCLC) or melanoma and previously found to lack susceptibility to treatment with an agent capable of inhibiting or reversing EMT or a chemotherapeutic agent (that is, non-responder subjects);
   (vi) a predetermined profile of LDH biomarker expression, activity, or amount “threshold” values obtained from a control subject or population of control subjects having non-small cell lung cancer (NSCLC) or melanoma and previously found to be susceptible to treatment with an agent capable of inhibiting or reversing EMT or a chemotherapeutic agent (that is, responder subjects); or
   (vii) obtained from a control sample having a known “threshold” value of LDH biomarker indicative of a control subject or population of control subjects having non-small cell lung cancer (NSCLC) or melanoma and previously found to be susceptible to treatment with an agent capable of inhibiting or reversing EMT or a chemotherapeutic agent (that is, responder subjects).
107. A method according to statement 105, wherein the control profile is:
   (i) a “threshold” value determined by: assessing LDH activity, expression, or amount in a population of subjects; determining which subjects in the population are susceptible to treatment with an agent capable of inhibiting or reversing EMT or a chemotherapeutic agent; and, selecting the lowest LDH activity, expression, or amount determined in a subject found to be susceptible to treatment with an agent capable of inhibiting or reversing EMT or a chemotherapeutic agent (that is, a responder subject) as the “threshold” value; or
   (ii) a “standard range” of values determined by: assessing LDH activity, expression, or amount in a population of subjects not having NSCLC and/or a population of subjects not having melanoma; and, determining a “standard range” of LDH activity, expression, or amount values in these subjects.
108. A method according to statement 106 or 107, wherein a higher activity, expression, or amount of LDH biomarker in the sample profile as compared with the control profile is indicative of susceptibility to treatment with an agent capable of inhibiting or reversing EMT or a chemotherapeutic agent.
109. A method according to any one of statements 101-108, wherein:
   said Lactate Dehydrogenase (LDH) biomarker comprises an amino acid sequence having at least 70%, 80%, 90%, 95%, 99% or 100% sequence identity with the full-length of the amino acid sequence disclosed at UniProt accession no: P00338-1, P00338-2, P00338-3, P00338-4, or P00338-5 (entry version 224) or a fragment thereof; and/or
   said Lactate Dehydrogenase (LDH) biomarker comprises an amino acid sequence having at least 70%, 80%, 90%, 95%, 99% or 100% sequence identity with the full-length of the amino acid sequence disclosed at UniProt accession no: P07195-1 (entry version 218) or a fragment thereof;
   or a nucleic acid (either DNA or RNA) having at least 70%, 80%, 90%, 95%, 99% or 100% sequence identity with the full-length nucleic acid sequence encoding said amino acid sequences.
110. A method according to any one of statements 105-109, wherein assessing the activity, expression, or amount of LDH biomarker in the subject, or in a sample derived from the subject, comprises:
   contacting a sample, or an extract from the sample, with a reagent for detecting the activity, expression, or amount of LDH biomarker, which reagent is selective for the LDH biomarker; and
   detecting and/or quantifying said reagent for detecting.
111. A method according to statement 110, wherein assessing the activity of LDH biomarker in the subject, or in a sample derived from the subject, comprises determining the enzymatic activity of LDH in the subject or in a sample derived from the subject;
   optionally wherein the enzymatic activity is assessed by determining the level or amount of a substrate or product of the enzymatic reaction catalysed by LDH.
112. A method according to statement 110, wherein assessing the expression or amount of LDH biomarker in the subject, or in a sample derived from the subject, comprises:
   contacting a sample, or an extract from the sample, with a specific binding member, which specific binding member selectively binds to LDH protein or mRNA; and
   detecting and/or quantifying formation of a complex formed by said specific binding member and said LDH protein or mRNA.
113. A method according to statement 112 wherein the specific binding member comprises an antibody molecule or binding fragment thereof.
114. A method according to statement 112 or 113, wherein detecting formation of a complex formed by the specific binding member and the LDH biomarker protein or mRNA is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.
115. A method according to any one of statements 112-114 wherein the expression or amount of the LDH biomarker is determined as the absolute amount of LDH biomarker protein or mRNA in the sample.
116. A method according to any one of statements 112-114 wherein the expression or amount of the LDH biomarker is determined as a relative amount of protein or mRNA in the sample,
   optionally wherein the relative amount is determined relative to a reference protein or mRNA in the sample, the expression of which is not altered by non-small cell lung cancer (NSCLC) or melanoma.
117. A method according to any one of statements 101-116, wherein the cancer-related outcome is susceptibility to treatment with an agent capable of inhibiting or reversing EMT or a chemotherapeutic agent.
118. A method according to statement 117, wherein the agent capable of inhibiting or reversing EMT or chemotherapeutic agent is an Axl inhibitor, Akt3 inhibitor, Slfn11 inhibitor, or PHGDH inhibitor.
119. A method according to statement 118, wherein the agent capable of inhibiting or reversing EMT or chemotherapeutic agent is an Axl inhibitor.
120. A method according to statement 119, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
121. A method according to statement 119, wherein the Axl inhibitor is BGB324/R428/bemcentinib.
122. A method according to statement 119, wherein the Axl inhibitor is an anti-Axl antibody described in WO2015/193428, WO2015/193430, WO2016/097370, or WO2016/166296.
123. A method according to statement 118, wherein the agent capable of inhibiting or reversing EMT or chemotherapeutic agent is an Akt3 inhibitor.
124. A method according to statement 123, wherein the Akt3 inhibitor is an Akt3 inhibitor disclosed in WO2016/102672.
125. A method according to any one of statements 117-124, wherein the agent capable of inhibiting or reversing EMT is administered as a single agent.
126. A method according to any one of statements 117-124, wherein the agent capable of inhibiting or reversing EMT is administered in combination with a further cancer treatment.
127. A method according to statement 126, wherein the further cancer treatment is selected from:
   (i) alkylating agents, including alkyl sulfonates such as busulfan;
   (ii) nitrogen mustards such as chlorambucil, cyclophosphamide, estramustine, ifosfamide, mechlorethamine, melphalan, and uramustine;
   (iii) ethyleneimine derivatives such as thiotepa;
   (iv) nitrosoureas such as carmustine, lomustine, and streptozocin;
   (v) triazenes such as dacarbazine, procarbazine, and temozolamide;
   (vi) platinum compounds such as cisplatin, carboplatin, oxaliplatin, satraplatin, and picoplatin onnaplatin, tetraplatin, sprioplatin, iproplatin, chloro(diethylenediamino)-platinum (II) chloride, dichloro(ethylenediamino)-platinum (II), diamino(2-ethylmalonato)platinum (II), (1,2-diaminocyclohexane)malonatoplatinum (II), (4-carboxyphthalo)-(1,2-diaminocyclohexane)platinum (II), (1,2-diaminocyclohexane)-(isocitrato)platinum (II), and (1,2-diaminocyclohexane)-cis-(pyruvato)platinum (II);
   (vii) antimetabolites, including antifolates such as methotrexate, permetrexed, raltitrexed, and trimetrexate;
   (viii) pyrimidine analogues such as azacitidine, capecitabine, cytarabine, edatrexate, floxuridine, fluorouracil, gemcitabine, and troxacitabine;
   (ix) purine analogues such as cladribine, chlorodeoxyadenosine, clofarabine, fludarabine, mercaptopurine, pentostatin, and thioguanine;
   (x) natural products, including antitumor antibiotics such as bleomycin, dactinomycin, mithramycin, mitomycin, mitoxantrone, porfiromycin;
   (xi) anthracyclines such as daunorubicin, doxorubicin, epirubicin, idarubicin, and valrubicin;
   (xii) mitotic inhibitors such as the vinca alkaloids vinblastine, vinvesir, vincristine, vindesine, and vinorelbine;
   (xiii) enzymes such as L-asparaginase and PEG-L-asparaginase;
   (xiv) microtubule polymer stabilizers such as the taxanes paclitaxel and docetaxel;
   (xv) topoisomerase I inhibitors such as the camptothecins irinotecan and topotecan;
   (xvi) topoisomerase II inhibitors such as podophyllotoxin, amsacrine, etoposide, teniposide, losoxantrone and actinomycin;
   (xvii) hormones and hormone antagonists, including
      androgens such as fluoxymesterone and testolactone;
      antiandrogens such as bicalutamide, cyproterone, flutamide, and nilutamide;
      corticosteroids such as dexamethasone and prednisone;
      aromatase inhibitors such as aminoglutethimide, anastrozole, exemestane, formestane, and letrozole;
      estrogens such as diethylstilbestrol;
      antiestrogens such as fulvestrant, raloxifene, tamoxifen, and toremifine;
      luteinising hormone-releasing hormone (LHRH) agonists and antagonists such as abarelix, buserelin, goserelin, leuprolide, histrelin, desorelin, nafarelin acetate and triptorelin;
      progestins such as medroxyprogesterone acetate and megestrol acetate; and
      thyroid hormones such as levothyroxine and liothyronine;
   (xviii) PKB pathway inhibitors, including perifosine, enzastaurin hydrochloride, and triciribine;
   (xix) P13K inhibitors such as semaphore and SF1126, and MTOR inhibitors such as rapamycin and analogues;
   (xx) CDK inhibitors, including seliciclib, alvocidib, and 7-hydroxystaurosporine;
   (xxi) COX-2 inhibitors, including celecoxib;
   (xxii) HDAC inhibitiors, including trichostatin A, suberoylanilide hydroxamic acid, and chlamydocin;
   (xxiii) DNA methylase inhibitors, including temozolomide, and miscellaneous agents, including altretamine, arsenic trioxide, thalidomide, lenalidomide, gallium nitrate, levamisole, mitotane, hydroxyurea, octreotide, procarbazine, suramin, photodynamic compounds such as methoxsalen and sodium porfimer;
   (xxiv) proteasome inhibitors such as bortezomib;
   (xxv) molecular targeted therapy agents including: functional therapeutic agents, including gene therapy agents and antisense therapy agents;
   (xxvi) tyrosine kinase inhibitors such as erlotinib hydrochloride, gefitinib, imatinib mesylate, and semaxanib;
   (xxvii) Raf inhibitors such as sorafenib, and gene expression modulators such as the retinoids and rexinoids, for example adapalene, bexarotene, trans-retinoic acid, 9-cis-retinoic acid, and N-(4-hydroxyphenyl)retinamide;
   (xxviii) phenotype-directed therapy agents, including monoclonal antibodies such as pembrolizumab, alemtuzumab, bevacizumab, cetuximab, ibritumomab tiuxetan, rituximab, and trastuzumab, immunotoxins such as gemtuzumab ozogamicin, radioimmunoconjugates such as I-tositumobab;
   (xxix) cancer vaccines;
   (xxx) biologic therapy agents including: interferons such as interferon-[alpha]2a and interferon-[alpha]2b, and interleukins such as aldesleukin, denileukin diftitox, and oprelvekin; (xxxi) anticancer therapies involving the use of protective or adjunctive agents, including: cytoprotective agents such as amifostine, and dexrazoxane, phosphonates such as pamidronate and zoledronic acid, and stimulating factors such as epoetin, darbeopetin, filgrastim, PEG-filgrastim, and sargramostim;
   (xxxii) Axl inhibitors such as 1-(6,7-dihydro-5H-benzo[6,7]cyclohepta[1,2-c]pyridazin-3-yl)-N3-((7-(S)-pyrrolidin-1-yl)-6,7,8,9-tetrahydro-5H-benzo[7]annulene-2-yl)-1H-1,2,4-triazole-3,5-diamine; and/or
   (xxxiii) further combination chemotherapeutic regimens, such as combinations of carboplatin/paclitaxel, capecitabine/docetaxel, fluorauracil/levamisole, fluorauracil/leucovorin, methotrexate/leucovorin, and trastuzumab/paclitaxel, alone or in further combination with carboplatin.
128. A method according to statement 126, wherein the further cancer treatment is a monoclonal antibody such as pembrolizumab, alemtuzumab, bevacizumab, cetuximab, ibritumomab tiuxetan, or rituximab, or is a B-Raf inhibitor such as debrafenib, a MEK inhibitor such as trametinib or a combination therapy of these agents.
129. A method according to statement 128, wherein the further cancer treatment is pembrolizumab.
130. A method according to statement 128, wherein the further cancer treatment is a combination therapy of debrafenib and trametinib.
131. A method according to any one of statements 101-130, wherein the subject is mammalian.
132. A method according to statement 131, wherein the subject is human.
133. A method according to any one of statements 101-132, wherein the predictive method is performed before the subject receives a treatment or course of treatment for non-small cell lung cancer (NSCLC) or melanoma.
134. A method according to any one of statement 101-133, wherein the predictive method is performed during or after a treatment or course of treatment for non-small cell lung cancer (NSCLC) or melanoma.
135. A method according to any one of statements 101-132 or 134, wherein the subject has previously been treated with an agent capable of inhibiting or reversing EMT or a chemotherapeutic agent.
136. A method according to statement 135, wherein the subject has previously been treated with an agent selected from: a PHGDH inhibitor, a Slfn11 inhibitor, an Axl inhibitor, or an Akt3 inhibitor.
137. A method according to any one of statements 135-136, wherein the subject has previously been treated with an agent that is different to the agent capable of inhibiting or reversing EMT or chemotherapeutic agent for which the subject's susceptibility is being predicted.
138. A method according to any one of statements 101-133, wherein the subject has not previously been treated with an agent capable of inhibiting or reversing EMT or a chemotherapeutic agent.
139. A method according to statement 138, wherein the subject has not previously been treated with an Axl inhibitor.
140. A method according to statement 138, wherein the subject has not previously been treated with an Akt3 inhibitor.
141. A method according to any one of statements 101-140, wherein the sample is selected from: blood, serum, plasma, tissue biopsy, culture supernatant, or urine.
142. A method according to statement 141, wherein the sample is a blood, serum, or plasma sample.
143. A method according to statement 142, wherein the sample is a serum sample.
144. A method according to statement 142, wherein the sample is a blood sample.
201. A method of selecting subjects having, suspected of having, or diagnosed with non-small cell lung cancer (NSCLC) or melanoma for treatment with an agent capable of inhibiting or reversing EMT or a chemotherapeutic agent, the method comprising:
   identifying subjects susceptible to treatment with an agent capable of inhibiting or reversing EMT or a chemotherapeutic agent using a method according to any one of statements 101-144; and
   selecting thus identified subjects for treatment.
202. A method according to statement 201, wherein treatment comprises administering to the subject a therapeutically effective amount of an agent capable of inhibiting or reversing EMT or a chemotherapeutic agent.
203. A method according to statement 202, wherein the treatment comprises an Axl inhibitor, Akt3 inhibitor, Slfn11 inhibitor, or PHGDH inhibitor.
204. A method according to statement 203, wherein the treatment comprises an Axl inhibitor.
205. A method according to statement 204, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
206. A method according to statement 205, wherein the Axl inhibitor is BGB324/R428/bemcentinib.
207. A method according to statement 205, wherein the Axl inhibitor is an anti-Axl antibody described in WO2015/193428, WO2015/193430, WO2016/097370, or WO2016/166296.
208. A method according to statement 203, wherein the treatment comprises an Akt3 inhibitor.
209. A method according to statement 208, wherein the Akt3 inhibitor is an Akt3 inhibitor disclosed in WO2016/102672.
210. A method according to any one of statements 202-209, wherein the treatment is administered as a single agent.
211. A method according to any one of statements 202-209, wherein the treatment is administered in combination with a further cancer treatment.
212. A method according to statement 211, wherein the further cancer treatment is selected from the further cancer treatments listed in statement 126.
213. A method according to statement 211, wherein the further cancer treatment is a monoclonal antibody such as pembrolizumab, alemtuzumab, bevacizumab, cetuximab, ibritumomab tiuxetan, or rituximab, or is a B-Raf inhibitor such as debrafenib, a MEK inhibitor such as trametinib or a combination therapy of these agents.
214. A method according to statement 213, wherein the further cancer treatment is pembrolizumab.
215. A method according to statement 213, wherein the further cancer treatment is a combination therapy of debrafenib and trametinib.
301. A diagnostic kit comprising a reagent for detecting the activity, expression, or amount of an LDH biomarker, which reagent for detecting is selective for LDH.
302. A test device comprising a reagent for detecting the activity, expression, or amount of an LDH biomarker, which reagent is selective for LDH.
303. A diagnostic kit or test device according to any one of statements 301 or 302, comprising:
   a specific binding member which selectively binds to an LDH biomarker; and
   one or more reagents for detecting said specific binding member, or one or more reagents for detecting and/or quantifying formation of a complex formed by said specific binding member and said LDH biomarker.
304. A diagnostic kit or test device according to statement 303, wherein the specific binding member comprises an antibody molecule or binding fragment thereof.
305. A diagnostic kit or test device according to statement 303 or 304, wherein detecting formation of a complex formed by the specific binding member and the biomarker is carried out by use of a technique selected from: Western blot; enzyme-linked immunosorbent assay (ELISA); radioimmunoassay (RIA); competitive enzyme immunoassay; double antibody sandwich ELISA (DAS-ELISA); liquid immunoarray technology; immunocytochemistry; immunohistochemistry; antibody microarray detection; precipitation of colloidal gold; affinity chromatography; ligand binding assay; and lectin binding assay.
306. A diagnostic kit or test device according to any one of statements 301-305, wherein the kit or test device comprises no more than 2, 5, 10, 15, 20, 25, 30, 40, or 50 reagents for detecting.
307. A diagnostic kit or test device according to any one of statements 301-306 for use in a method of predicting a cancer-related outcome in a subject.
308. A diagnostic kit for use, or test device for use, according to statement 307, wherein the cancer-related outcome is susceptibility to treatment with an agent capable of inhibiting or reversing EMT or a chemotherapeutic agent.
309. A diagnostic kit for use, or test device for use, according to statement 308, wherein the agent capable of inhibiting or reversing EMT or chemotherapeutic agent is an Axl inhibitor, Akt3 inhibitor, Slfn11 inhibitor, or PHGDH inhibitor.
310. A diagnostic kit for use, or test device for use, according to statement 308, wherein the agent capable of inhibiting or reversing EMT or chemotherapeutic agent is an Axl inhibitor.
311. A diagnostic kit for use, or test device for use, according to statement 310, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
312. A diagnostic kit for use, or test device for use, according to statement 311, wherein the Axl inhibitor is BGB324/R428/bemcentinib.
313. A diagnostic kit for use, or test device for use, according to statement 311, wherein the Axl inhibitor is an anti-Axl antibody described in WO2015/193428, WO2015/193430, WO2016/097370, or WO2016/166296.
314. A diagnostic kit for use, or test device for use, according to statement 308, wherein the agent capable of inhibiting or reversing EMT or chemotherapeutic agent is an Akt3 inhibitor.
315. A diagnostic kit for use, or test device for use, according to any one of statements 307-310, wherein the subject has, is suspected of having, or has been diagnosed with non-small cell lung cancer (NSCLC) or melanoma.
316. A diagnostic kit for use, or test device for use, according to any one of statements 307-315, wherein the method is a method according to any one of statements 101-144.
317. Use of a reagent for detecting the activity, expression, or amount of an LDH biomarker in a method of predicting a cancer-related outcome in a subject.
318. Use of a reagent for detecting the activity, expression, or amount an LDH biomarker, which reagent for detecting is selective for LDH, in a method of manufacture of a diagnostic kit or test device for use in a method of predicting a cancer-related outcome in a subject.
319. A use according to statement 317 or 318, wherein the reagent for detecting comprises a specific binding member selective for an LDH biomarker.
320. A use according to statement 317 or 318, wherein the method of predicting a cancer-related outcome in a subject is a method according to any one of statements 101-144.
401. Use of LDH as a biomarker for determining if a subject is susceptible to treatment with an agent capable of inhibiting or reversing EMT or a chemotherapeutic agent.
402. A use according to statement 401, wherein:
   (i) a higher activity, expression, or amount of said LDH biomarker in the subject, or sample derived from the subject, compared with a control profile for the respective biomarker is indicative of susceptibility to treatment with an agent capable of inhibiting or reversing EMT or a chemotherapeutic agent; or
   (ii) a lower activity, expression, or amount of said LDH biomarker in the subject, or sample derived from the subject, compared with a control profile for the respective biomarker is indicative of susceptibility to treatment with an agent capable of inhibiting or reversing EMT or a chemotherapeutic agent.
403. A use according to any one of statements 401-402, wherein the use is as a biomarker in a method according to any one of statements 101-144.
501. A method of treating a subject having, suspected of having, or diagnosed with non-small cell lung cancer (NSCLC) or melanoma, the method comprising:
   identifying a subject susceptible to treatment using a method according to any one of statements 101-144; and
   administering to the subject a therapeutically effective amount of an agent capable of inhibiting or reversing EMT or a chemotherapeutic agent.
502. A method of treating a subject having, suspected of having, or diagnosed with non-small cell lung cancer (NSCLC) or melanoma, the method comprising:
   (a) obtaining a sample from the subject;
   (b) determining if the subject is susceptible to treatment with an agent capable of inhibiting or reversing EMT or a chemotherapeutic agent using a method according to any one of statements 101-144;
   (c) administering to the subject a therapeutically effective amount of an agent capable of inhibiting or reversing EMT or a chemotherapeutic agent.
503. A method of treating a subject having, suspected of having, or diagnosed with non-small cell lung cancer (NSCLC) or melanoma, the method comprising administering a therapeutically effective amount of an agent capable of inhibiting or reversing EMT or chemotherapeutic agent to a subject identified as susceptible to said treatment using a method according to any one of statements 101-144.
504. An agent capable of inhibiting or reversing EMT or chemotherapeutic agent for use in a method of treating a subject having, suspected of having, or diagnosed with non-small cell lung cancer (NSCLC) or melanoma,
   wherein the method is a method according to any one of statements 501-503.
505. Use of an agent capable of inhibiting or reversing EMT or chemotherapeutic agent for use in a method of manufacture of a medicament for use in a method of treating a subject having, suspected of having, or diagnosed with non-small cell lung cancer (NSCLC) or melanoma,
   wherein the method of treatment is a method according to any one of statements 501-503.
506. A method, agent for use, or use according to any one of statements 501-505, wherein the agent capable of inhibiting or reversing EMT or chemotherapeutic agent comprises an Axl inhibitor, Akt3 inhibitor, Slfn11 inhibitor, or PHGDH inhibitor.
507. A method, agent for use, or use according to statement 506, wherein the agent capable of inhibiting or reversing EMT or chemotherapeutic agent comprises an Axl inhibitor.
508. A method, agent for use, or use according to statement 507, wherein the Axl inhibitor is BGB324/R428/bemcentinib, cabozantinib, TP-0903, foretinib, merestinib, bosutinib, gilteritinib, crizotinib, amuvatinib, sunitinib, sitravatinib, LDC1267, UNC2025, or an anti-Axl antibody described in WO2015/193428, WO2015/193430, WO2016/097370, or WO2016/166296.
509. A method, agent for use, or use according to statement 507, wherein the Axl inhibitor is BGB324/R428/bemcentinib.
510. A method, agent for use, or use according to statement 507, wherein the Axl inhibitor is an anti-Axl antibody described in WO2015/193428, WO2015/193430, WO2016/097370, or WO2016/166296.
511. A method, agent for use, or use according to statement 506, wherein the agent capable of inhibiting or reversing EMT or chemotherapeutic agent comprises an Akt3 inhibitor.
512. A method, agent for use, or use according to statement 511, wherein the Akt3 inhibitor is an Akt3 inhibitor disclosed in WO2016/102672.
513. A method, agent for use, or use according to any one of statements 501-512, wherein the agent capable of inhibiting or reversing EMT or chemotherapeutic agent is administered as a single agent.
514. A method, agent for use, or use according to any one of statements 501-512, wherein the agent capable of inhibiting or reversing EMT or chemotherapeutic agent is administered in combination with a further cancer treatment.
515. A method, agent for use, or use according to statement 514, wherein the further cancer treatment is selected from the further cancer treatments listed in statement 126.
516. A method, agent for use, or use according to statement 514, wherein the further cancer treatment is a monoclonal antibody such as pembrolizumab, alemtuzumab, bevacizumab, cetuximab, ibritumomab tiuxetan, or rituximab, or is a B-Raf inhibitor such as debrafenib, a MEK inhibitor such as trametinib or a combination therapy of these agents.
517. A method, agent for use, or use according to statement 516, wherein the further cancer treatment is pembrolizumab.
518. A method, agent for use, or use according to statement 516, wherein the further cancer treatment is a combination therapy of debrafenib and trametinib.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the disclosure in diverse forms thereof.

While the disclosure has been described in conjunction with the exemplary embodiments described above, many equivalent modifications and variations will be apparent to those skilled in the art when given this disclosure. Accordingly, the exemplary embodiments of the disclosure set forth above are considered to be illustrative and not limiting. Various changes to the described embodiments may be made without departing from the spirit and scope of the disclosure.

For the avoidance of any doubt, any theoretical explanations provided herein are provided for the purposes of improving the understanding of a reader. The authors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

Serum samples were obtained from cohorts of cancer patients both before and once after these subjects were administered a drug treatment of Axl inhibitor, either alone or in combination with another agent. Subjects in these cohorts were categorised as either “responders” or “non-responders” based on response to the administered treatment.

Subsequently, the serum samples were analysed by a contract research organization (CRO) using the DiscoveryMAP v 3.3 soluble protein panel to measure the levels of soluble protein markers (281 proteins in total) in serum samples, and statistical analysis of this protein panel data was performed to identify differentially expressed proteins in the responder and non-responder groups.

In these comparative analyses pre-treatment (C1D1) differences in protein panel data were identified between responder and non-responder groups: prior to treatment certain biomarkers are present in the serum of “responder” subjects at higher levels than in the serum of “non-responder” subjects. Similarly, prior to treatment certain biomarkers are present in the serum of “responder” subjects at lower levels than in the serum of “non-responder” subjects.

Thus, the present authors have identified multiple serum based soluble protein biomarkers that are predictive of patient benefit following treatment with Axl inhibitors.

### Example 1

This example describes the results from analyses of trial BGBC003 (NCT02488408), which consists of samples from patients diagnosed with Acute Myeloid Leukaemia (AML) and Myelodysplastic Syndrome (MDS). Subjects were administered the Axl inhibitor BGB324 either as a single agent or in combination with cytarabine or decitabine.

The protein panel dataset was split into two subsets, one subset with only AML samples, and another with only MDS samples. Pre-treatment (C1D1) differences in protein expression were identified between responder and non-responder groups by statistical analysis of the protein panel data.

A first analysis identified Axl as a biomarker which was significantly down-regulated in responders as compared with non-responders prior to treatment with Axl inhibitor (p=0.006), and found that Axl levels increase in responders but not non-responders following treatment with an Axl inhibitor.

A subsequent analysis identified a further 12 proteins in the AML subset as significantly different (p<0.05) between responders and non-responders in the pre-treatment samples (log FC=Log 2 fold change of the protein; a positive value indicates up regulation, while a negative value indicates down regulation in the comparison of interest):

| | | | P. | adj. | |
|---|---|---|---|---|---|
| Biomarker ID | FC | logFC | Value | P. Val | |
| Haptoglobin | 6.973 | 2.802 | 0.009401 | 0.9861 | up |
| Fibroblast Growth | −4.877 | −2.286 | 0.0148 | 0.9861 | down |
| Factor 21 (FGF-21) | | | | | |
| Receptor for advanced | −2.676 | −1.42 | 0.01642 | 0.9861 | down |
| glycosylation end | | | | | |
| products (RAGE) | | | | | |
| Carcinoembryonic | −2.147 | −1.102 | 0.01764 | 0.9861 | down |
| Antigen (CEA) | | | | | |
| Follicle-Stimulating | −2.818 | −1.495 | 0.02104 | 0.9861 | down |
| Hormone (FSH) | | | | | |
| Matrix | −1.881 | −0.911 | 0.02581 | 0.9861 | down |
| Metalloproteinase-10 | | | | | |
| (MMP-10) | | | | | |
| Neutrophil Activating | 3.271 | 1.71 | 0.03312 | 0.9861 | up |
| Peptide 2 (NAP-2) | | | | | |
| Immunoglobulin | 2.611 | 1.385 | 0.03414 | 0.9861 | up |
| E (IgE) | | | | | |
| Omentin | −1.795 | −0.844 | 0.03658 | 0.9861 | down |
| CD40 Ligand | 2.877 | 1.525 | 0.04076 | 0.9861 | up |
| (CD40-L) | | | | | |
| Cancer Antigen | −5.153 | −2.365 | 0.04178 | 0.9861 | down |
| 19-9 (CA-19-9) | | | | | |
| Luteinizing | −2.136 | −1.095 | 0.04562 | 0.9861 | down |
| Hormone (LH) | | | | | |

16 proteins were identified in the MDS subset as significantly different (p<0.05) between responders and non-responders in the pre-treatment samples (log FC=Log 2 fold change of the protein; a positive value indicates up regulation, while a negative value indicates down regulation in the comparison of interest):

| | | | P. | adj. | |
|---|---|---|---|---|---|
| Biomarker ID | FC | logFC | Value | P. Val | |
| Tamm-Horsfall Urinary | 4.949 | 2.307 | 0.0001461 | 0.04106 | up |
| Glycoprotein (THP) | | | | | |
| TNF-Related Apoptosis- | 3.139 | 1.65 | 0.0003568 | 0.05013 | up |
| Inducing Ligand | | | | | |
| Receptor 3 (TRAIL-R3) | | | | | |
| Transferrin receptor | −4.02 | −2.007 | 0.00185 | 0.1733 | down |
| protein 1 | | | | | |
| FASLG Receptor (FAS) | −3.503 | −1.809 | 0.005781 | 0.4061 | down |
| Macrophage-Derived | 4.945 | 2.306 | 0.007458 | 0.4191 | up |
| Chemokine | | | | | |
| Antileukoproteinase | 1.406 | 0.491 | 0.01516 | 0.6529 | up |
| (ALP) | | | | | |
| Trefoil Factor | 2.292 | 1.196 | 0.01626 | 0.6529 | up |
| 3 (TFF3) | | | | | |
| Fatty Acid-Binding | 2.543 | 1.347 | 0.02218 | 0.6569 | up |
| Protein, adipocyte | | | | | |
| (FABP, adipocyte) | | | | | |
| von Willebrand | 4.858 | 2.28 | 0.02423 | 0.6569 | up |
| Factor (vWF) | | | | | |
| Vitamin D-Binding | 1.82 | 0.864 | 0.0248 | 0.6569 | up |
| Protein (VDBP) | | | | | |
| Cystatin-B | 1.618 | 0.694 | 0.02571 | 0.6569 | up |
| Uteroglobin | 3.804 | 1.927 | 0.03509 | 0.8217 | up |
| Fibrinogen | 1.679 | 0.748 | 0.03906 | 0.8374 | up |
| Interleukin-8 (IL-8) | −6.695 | −2.743 | 0.04371 | 0.8374 | down |
| EGFR | 1.283 | 0.36 | 0.0493 | 0.8374 | up |
| Leptin | 4.473 | 2.161 | 0.04959 | 0.8374 | up |

7 proteins were identified in the combined (AML and MDS) set as significantly different (p<0.05) between responders and non-responders in the pre-treatment samples (log FC=Log 2 fold change of the protein; a positive value indicates up regulation, while a negative value indicates down regulation in the comparison of interest):

| | | | P. | adj. | |
|---|---|---|---|---|---|
| Biomarker ID | FC | logFC | Value | P. Val | |
| Tamm-Horsfall Urinary | 1.841 | 0.88 | 0.0113 | 0.99650 | up |
| Glycoprotein (THP) | | | | | |
| Fibroblast Growth | −3.226 | −1.69 | 0.0164 | 0.99650 | down |
| Factor 21 (FGF-21) | | | | | |
| Matrix | −1.753 | −0.81 | 0.0190 | 0.99650 | down |
| Metalloproteinase-10 | | | | | |
| (MMP-10) | | | | | |
| Transferrin receptor | −1.757 | −0.813 | 0.0207 | 0.99650 | down |
| protein 1 (TFR1) | | | | | |
| Hemopexin | 1.307 | 0.386 | 0.0420 | 0.99650 | up |
| Haptoglobin | 6.34 | 2.664 | 0.0456 | 0.99650 | up |
| Immunoglobulin | 2.284 | 1.192 | 0.0499 | 0.99650 | up |
| M (IgM) | | | | | |

### Example 2

This example describes the results from analysis of trial BGBIL006 (NCT02872259), which consists of samples from patients diagnosed with melanoma. Subjects were administered the Axl inhibitor BGB324 in combination with either pembrolizumab or dabrafenib and trametinib.

14 proteins were identified as significantly different (p<0.05) between responders and non-responders in the pre-treatment samples (log FC=Log 2 fold change of the protein; a positive value indicates up regulation, while a negative value indicates down regulation in the comparison of interest):

| | | | P. | adj. | |
|---|---|---|---|---|---|
| Biomarker ID | FC | logFC | Value | P. Val | |
| Interleukin-6 receptor | −1.492 | −0.577 | 0.001992 | 0.5598 | down |
| (IL-6r) | | | | | |
| Tissue Inhibitor of | 1.451 | 0.537 | 0.007126 | 0.6736 | up |
| Metalloproteinases | | | | | |
| 1 (TIMP-1) | | | | | |
| Heparin-Binding EGF- | 1.472 | 0.558 | 0.01055 | 0.6736 | up |
| Like Growth Factor | | | | | |
| (HB-EGF) | | | | | |
| Antileukoproteinase | 1.253 | 0.325 | 0.01084 | 0.6736 | up |
| (ALP) | | | | | |
| Latency-Associated | 1.827 | 0.869 | 0.01222 | 0.6736 | up |
| Peptide of Transforming | | | | | |
| Growth Factor beta | | | | | |
| 1 (LAP TGF-b1) | | | | | |
| Epidermal Growth | 2.115 | 1.081 | 0.01656 | 0.6736 | up |
| Factor (EGF) | | | | | |
| Platelet-Derived Growth | 2.299 | 1.201 | 0.01726 | 0.6736 | up |
| Factor BB (PDGF-BB) | | | | | |
| Osteoprotegerin (OPG) | 1.502 | 0.587 | 0.01918 | 0.6736 | up |
| Neutrophil Activating | 1.67 | 0.74 | 0.02807 | 0.7262 | up |
| Peptide 2 (NAP-2) | | | | | |
| Chemokine CC-4 | −1.407 | −0.493 | 0.02965 | 0.7262 | down |
| (HCC-4) | | | | | |
| ST2 | 1.721 | 0.783 | 0.03301 | 0.7262 | up |
| Tumor Necrosis Factor | 1.556 | 0.638 | 0.03657 | 0.7262 | up |
| Receptor I (TNF RI) | | | | | |
| Vascular Endothelial | 1.862 | 0.897 | 0.03698 | 0.7262 | up |
| Growth Factor (VEGF) | | | | | |
| Growth-Regulated alpha | 1.72 | 0.782 | 0.0375 | 0.7262 | up |
| protein (GRO-alpha) | | | | | |

### Example 3

This example describes the results from analysis of trial BGBC007 (NCT03184558), which consists of samples from patients diagnosed with melanoma. Subjects were administered the Axl inhibitor BGB324 in combination with pembrolizumab.

8 proteins were identified as significantly different (p<0.05) between responders and non-responders in the pre-treatment samples (log FC=Log 2 fold change of the protein; a positive value indicates up regulation, while a negative value indicates down regulation in the comparison of interest):

| | | | P. | adj. | |
|---|---|---|---|---|---|
| Biomarker ID | FC | logFC | Value | P. Val | |
| Interleukin-23 (IL-23) | 1.699 | 0.765 | 0.006719 | 0.5822 | up |
| Folate receptor | 6.235 | 2.64 | 0.007624 | 0.5822 | up |
| gamma (FOLR3) | | | | | |
| Interleukin-6 receptor | −1.955 | −0.967 | 0.008476 | 0.5822 | down |
| subunit beta (IL-6R beta) | | | | | |
| Clusterin (CLU) | 1.596 | 0.674 | 0.009641 | 0.5822 | up |
| Intercellular Adhesion | −2.957 | −1.564 | 0.01036 | 0.5822 | down |
| Molecule 1 (ICAM-1) | | | | | |
| Fas Ligand (FasL) | 2.337 | 1.225 | 0.01244 | 0.5825 | up |
| Macrophage | −4.287 | −2.1 | 0.02594 | 1 | down |
| Inflammatory | | | | | |
| Protein-1 beta | | | | | |
| (MIP-1 beta) | | | | | |
| Periostin | −3.803 | −1.927 | 0.04617 | 1 | down |

### Example 4

This example describes the results from analysis of trial BGBC008 (NCT03184571), which consists of samples from patients diagnosed with NSCLC. Subjects were administered the Axl inhibitor BGB324 in combination with pembrolizumab.

17 proteins were identified as significantly different (p<0.05) between responders and non-responders in the pre-treatment samples (log FC=Log 2 fold change of the protein; a positive value indicates up regulation, while a negative value indicates down regulation in the comparison of interest):

| | | | P. | adj. | |
|---|---|---|---|---|---|
| Biomarker ID | FC | logFC | Value | P. Val | |
| Neuronal Cell Adhesion | 3.919 | 1.97 | 0.0001822 | 0.05119 | up |
| Molecule (Nr-CAM) | | | | | |
| Beta Amyloid | −2.826 | −1.499 | 0.007978 | 0.6786 | down |
| 1-40 (AB-40) | | | | | |
| Sclerostin | 2.072 | 1.051 | 0.01051 | 0.6786 | up |
| Aggrecan core | −1.118 | −0.16 | 0.01283 | 0.6786 | down |
| protein (Aggrecan) | | | | | |
| Prostate-Specific | 94.617 | 6.564 | 0.01456 | 0.6786 | up |
| Antigen, Free (PSA-f) | | | | | |
| Epidermal Growth Factor | 1.404 | 0.49 | 0.01939 | 0.6786 | up |
| Receptor (EGFR) | | | | | |
| Neurofilament heavy | 2.653 | 1.408 | 0.02215 | 0.6786 | up |
| polypeptide (NF-H) | | | | | |
| Ferritin (FRTN) | −8.136 | −3.024 | 0.0224 | 0.6786 | down |
| Receptor for advanced | −9.265 | −3.212 | 0.02571 | 0.6786 | down |
| glycosylation end | | | | | |
| products (RAGE) | | | | | |
| Dopamine beta- | 4.841 | 2.275 | 0.02934 | 0.6786 | up |
| hydroxylase (DBH) | | | | | |
| Fibulin-1C (Fib-1C) | 2.492 | 1.317 | 0.02939 | 0.6786 | up |
| Myoglobin | 4.394 | 2.135 | 0.03025 | 0.6786 | up |
| Urokinase-type | 1.631 | 0.706 | 0.03318 | 0.6786 | up |
| Plasminogen | | | | | |
| Activator (uPA) | | | | | |
| Visceral adipose tissue - | 3.013 | 1.591 | 0.03489 | 0.6786 | up |
| derived serpin | | | | | |
| A12 (Vaspin) | | | | | |
| Interleukin-23 (IL-23) | 1.467 | 0.553 | 0.03623 | 0.6786 | up |
| Alpha-2-Macroglobulin | 1.467 | 0.553 | 0.03879 | 0.6813 | up |
| (A2Macro) | | | | | |
| Tissue Inhibitor of | 1.139 | 0.188 | 0.04656 | 0.7696 | up |
| Metalloproteinases | | | | | |
| 2 (TIMP-2) | | | | | |

### Example 5

This example describes the results from analysis of trial BGBIL005 (NCT02922777), which consists of samples from patients diagnosed with NSCLC. Subjects were administered the Axl inhibitor BGB324 in combination with docetaxel.

8 proteins were identified as significantly different (p<0.05) between responders and non-responders in the pre-treatment samples (log FC=Log 2 fold change of the protein; a positive value indicates up regulation, while a negative value indicates down regulation in the comparison of interest):

| | | | P. | adj. | |
|---|---|---|---|---|---|
| Biomarker ID | FC | logFC | Value | P. Val | |
| Myeloid Progenitor | 2.515 | 1.33 | 0.0004716 | 0.1325 | up |
| Inhibitory Factor 1 | | | | | |
| (MPIF-1) | | | | | |
| Pancreatic | 5.255 | 2.394 | 0.01592 | 1 | up |
| Polypeptide (PPP) | | | | | |
| Visceral adipose tissue - | 5.786 | 2.533 | 0.02239 | 1 | up |
| derived serpin | | | | | |
| A12 (Vaspin) | | | | | |
| Thyroid-Stimulating | −4.362 | −2.125 | 0.02275 | 1 | down |
| Hormone | | | | | |
| Interleukin-7 (IL-7) | 1.254 | 0.327 | 0.03036 | 1 | up |
| Pulmonary and | 1.57 | 0.651 | 0.03059 | 1 | up |
| Activation-Regulated | | | | | |
| Chemokine (PARC) | | | | | |
| Cancer Antigen | 4.86 | 2.281 | 0.03891 | 1 | up |
| 15-3 (CA-15-3) | | | | | |
| Tetranectin | −1.294 | −0.372 | 0.04522 | 1 | down |

### Example 6

This example describes the results from analysis of trial BGBC004 (NCT02424617), which consists of samples from patients diagnosed with NSCLC. Subjects were administered the Axl inhibitor BGB324 in combination with erlotinib.

9 proteins were identified as significantly different (p<0.05) between responders and non-responders in the pre-treatment samples (log FC=Log 2 fold change of the protein; a positive value indicates up regulation, while a negative value indicates down regulation in the comparison of interest):

| | | | P. | adj. | |
|---|---|---|---|---|---|
| Biomarker ID | FC | logFC | Value | P. Val | |
| Pepsinogen I (PGI) | −2.029 | −1.021 | 0.002814 | 0.7907 | down |
| Apolipoprotein E | −2.524 | −1.336 | 0.006299 | 0.8575 | down |
| (Apo E) | | | | | |
| Apolipoprotein C-I | −1.381 | −0.466 | 0.01258 | 0.8575 | down |
| (Apo C-I) | | | | | |
| Vitamin D-Binding | 1.795 | 0.844 | 0.01442 | 0.8575 | up |
| Protein (VDBP) | | | | | |
| Macrophage-Stimulating | −2.406 | −1.267 | 0.01756 | 0.8575 | down |
| Protein | | | | | |
| C-C motif chemokine | −1.904 | −0.929 | 0.02042 | 0.8575 | down |
| 15 (CCL15) | | | | | |
| Progranulin | −1.78 | −0.832 | 0.023 | 0.8575 | down |
| Carcinoembryonic | −1.772 | −0.825 | 0.02441 | 0.8575 | down |
| antigen-related cell | | | | | |
| adhesion molecule | | | | | |
| 1 (CEACAM1) | | | | | |
| Carbonic anhydrase | 1.878 | 0.909 | 0.03379 | 0.9893 | up |
| 9 (CA-9) | | | | | |

### Example 7

This example describes the results from analysis of trial BGBIL006 (NCT02872259), which consists of samples from patients diagnosed with melanoma, and trial BGBC008 (NCT03184571), which consists of samples from patients diagnosed with NSCLC. Subjects were administered the Axl inhibitor BGB324 in combination with either pembrolizumab or dabrafenib and trametinib.

Samples from a total of 29 patients were collected and analysed (7 from NCT02872259 and 22 from NCT03184571). Pre-treatment levels of the blood-based biomarker Lactate Dehydrogenase (LDH) were measured by clinical laboratories as part of the clinical trial screening process, and entered into the electronic case report form (eCRF). Subjects were administered the Axl inhibitor BGB324 in combination with either pembrolizumab or dabrafenib and trametinib, and the response was measured by RECIST (Response Evaluation Criteria in Solid Tumors) criteria.

LDH protein was found to be significantly different between responders and non-responders in the pre-treatment samples. Using a preliminary cut-off of 287 units per litre (U/L), patients can be classified as responders and non-responders with a test accuracy of 79%. A “normal” LDH level is considered to be between 115-255 U/L:

| | Screen | | | | | | | | |
|---|---|---|---|---|---|---|---|---|---|
| | LDH | | Test | Correct | True | False | True | False | LDH |
| ID | Levels | Responder | Result | Call | Responder | Responder | NR | NR | cutoff |
| 233003 | 135 | No | No | TRUE | | | TNR | | 287 |
| 232004 | 148 | No | No | TRUE | | | TNR | | |
| 233001 | 159 | No | No | TRUE | | | TNR | | |
| 01-006 | 162 | No | No | TRUE | | | TNR | | |
| 236001 | 189 | No | No | TRUE | | | TNR | | |
| 236003 | 192 | No | No | TRUE | | | TNR | | |
| 232003 | 205 | No | No | TRUE | | | TNR | | |
| 221003 | 209 | No | No | TRUE | | | TNR | | |
| 01-020 | 209 | No | No | TRUE | | | TNR | | |
| 01-012 | 233 | No | No | TRUE | | | TNR | | |
| 01-017 | 234 | Yes | No | FALSE | | | | FNR | |
| 201001 | 241 | No | No | TRUE | | | TNR | | |
| 01-016 | 246 | No | No | TRUE | | | TNR | | |
| 211001 | 250 | No | No | TRUE | | | TNR | | |
| 234004 | 264 | No | No | TRUE | | | TNR | | |
| 221001 | 274 | No | No | TRUE | | | TNR | | |
| 234002 | 277 | No | No | TRUE | | | TNR | | |
| 212002 | 277 | No | No | TRUE | | | TNR | | |
| 01-010 | 288 | Yes | Yes | TRUE | TR | | | | |
| 237002 | 298 | Yes | Yes | TRUE | TR | | | | |
| 212005 | 351 | Yes | Yes | TRUE | TR | | | | |
| 01-003 | 381 | No | Yes | FALSE | | FR | | | |
| 234006 | 402 | No | Yes | FALSE | | FR | | | |
| 212001 | 409 | Yes | Yes | TRUE | TR | | | | |
| 234005 | 461 | Yes | Yes | TRUE | TR | | | | |
| 237001 | 463 | Yes | Yes | TRUE | TR | | | | |
| 234003 | 482 | No | Yes | FALSE | | FR | | | |
| 234007 | 669 | No | Yes | FALSE | | FR | | | |
| 231001 | 828 | No | Yes | FALSE | | FR | | | |
| | | | | | 6 | 5 | 17 | 1 | |

| | | |
|---|---|---|
| True | No correctly identified | 6 |
| Responder | as responder | |
| False | No incorrectly identified | 5 |
| Responder | as responder | |
| True Non | No correctly identified | 17 |
| Responder | as non responder | |
| False Non | No incorrectly identified | 1 |
| Responder | as non responder | |

| | | |
|---|---|---|
| Accuracy | 79% | Overall accuracy of test |
| Sensitivity | 86% | Probability test result will be Responder when a Responder is |
| | | tested |
| Specificity | 77% | Probability test result will be Non Responder when a Non |
| | | Responder is tested |
| NPV | 94% | Probability that the patient is a Non Responder when the test |
| | | gives a Non Responder Result |
| PPV | 55% | Probability that the patient is a Responder when the test gives |
| | | a Responder Result |

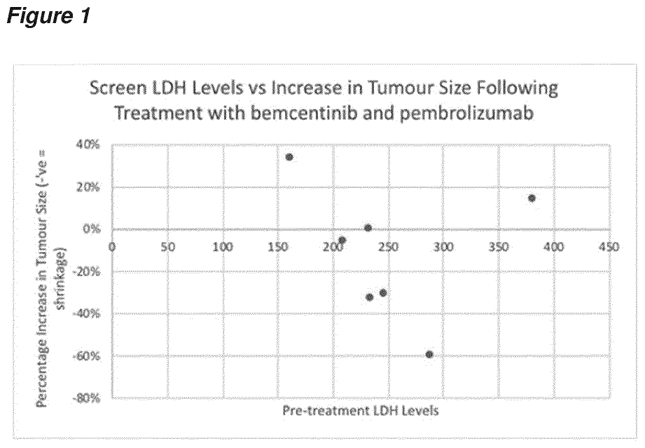

In addition, data was analysed from trial BGBIL006 (NCT02872259) to investigate the correlation between pre-treatment LDH levels and increase/decrease in tumour size following treatment with BGB324 and pembrolizumab. The results of this analysis are shown in FIG. 1.

### Example 8

This example describes the results from further analyses of data from trial BGBC003 (NCT02488408), which consists of samples from patients diagnosed with Acute Myeloid Leukaemia (AML) and Myelodysplastic Syndrome (MDS). Subjects were administered the Axl inhibitor BGB324 either as a single agent or in combination with cytarabine or decitabine.

The protein panel dataset was split into two subsets, one subset with only AML samples, and another with only MDS samples. Pre-treatment (C1D1) differences in protein expression were identified between responder and non-responder groups by statistical analysis of the protein panel data.

The analysis identified 10 proteins in the combined AML and MDS monotherapy subset as significantly different (p<0.05) between responders and non-responders in the pre-treatment samples (log FC=Log 2 fold change of the protein; a positive value indicates up regulation, while a negative value indicates down regulation in the comparison of interest):

| | | | P. | adj. | |
|---|---|---|---|---|---|
| Biomarker ID | FC | logFC | Value | P. Val | |
| Vitamin D-Binding Protein | 2.045 | 1.032 | 0.002 | 0.480 | up |
| Growth/differentiation | −2.006 | −1.005 | 0.016 | 0.856 | down |
| factor 15 (GDF-15) | | | | | |
| EGFR | 1.294 | 0.372 | 0.018 | 0.856 | up |
| Epithelial cell adhesion | 2.457 | 1.297 | 0.019 | 0.856 | up |
| molecule (EpCam) | | | | | |
| Tamm-Horsfall Urinary | 1.665 | 0.735 | 0.023 | 0.856 | up |
| Glycoprotein (THP) | | | | | |
| Macrophage-Stimulating | 1.605 | 0.682 | 0.025 | 0.856 | up |
| Protein | | | | | |
| Paraoxonase-1 (PON-1) | 1.660 | 0.731 | 0.036 | 0.856 | up |
| Fetuin-A | 1.268 | 0.343 | 0.040 | 0.856 | up |
| C-Peptide | −1.773 | −0.826 | 0.045 | 0.856 | down |
| Apolipoprotein A-II | 1.309 | 0.389 | 0.045 | 0.856 | up |
| (Apo A-II) | | | | | |

The analysis identified 5 proteins in the combined AML and MDS combination therapy subset as significantly different (p<0.05) between responders and non-responders in the pre-treatment samples (log FC=Log 2 fold change of the protein; a positive value indicates up regulation, while a negative value indicates down regulation in the comparison of interest):

| | | | P. | adj. | |
|---|---|---|---|---|---|
| Biomarker ID | FC | logFC | Value | P. Val | |
| Tamm-Horsfall Urinary | 1.362 | 0.446 | 0.012 | 1 | up |
| Glycoprotein (THP) | | | | | |
| Vitamin D-Binding | 1.405 | 0.490 | 0.013 | 1 | up |
| Protein (VDBP) | | | | | |
| Interleukin-6 receptor | 1.159 | 0.213 | 0.028 | 1 | up |
| subunit beta | | | | | |
| Complement Factor H - | −2.540 | −1.345 | 0.031 | 1 | down |
| Related Protein 1 (CFHR1) | | | | | |
| Epithelial cell adhesion | 1.664 | 0.734 | 0.032 | 1 | up |
| molecule (EpCam) | | | | | |

### Example 9

This example describes the results from analysis of trial BGBIL006 (NCT02872259), which consists of samples from patients diagnosed with melanoma. Subjects were administered the Axl inhibitor BGB324 in combination with either pembrolizumab or dabrafenib and trametinib.

The protein panel dataset was split into two subsets, one subset with only the pembrolizumab combination samples, and another with all patient samples. Pre-treatment (C1D1) differences in protein expression were identified between responder and non-responder groups by statistical analysis of the protein panel data.

The analysis identified 19 proteins in the pembrolizumab subset as significantly different (p<0.05) between responders and non-responders in the pre-treatment samples (log FC=Log 2 fold change of the protein; a positive value indicates up regulation, while a negative value indicates down regulation in the comparison of interest):

| | | | P. | adj. | |
|---|---|---|---|---|---|
| Biomarker ID | FC | logFC | Value | P. Val | |
| Pancreatic secretory trypsin | 1.674 | 0.743 | 0.002 | 0.290 | up |
| inhibitor (TATI) | | | | | |
| Chromogranin-A (CgA) | 2.680 | 1.422 | 0.002 | 0.290 | up |
| Matrix Metalloproteinase-7 | 2.029 | 1.020 | 0.003 | 0.290 | up |
| (MMP-7) | | | | | |
| Kidney Injury Molecule-1 | 1.625 | 0.701 | 0.011 | 0.482 | up |
| (KIM-1) | | | | | |
| Neuronal Cell Adhesion | 1.390 | 0.475 | 0.012 | 0.482 | up |
| Molecule (Nr-CAM) | | | | | |
| Leptin | 3.140 | 1.651 | 0.013 | 0.482 | up |
| Heparin-Binding EGF-Like | 1.500 | 0.583 | 0.013 | 0.482 | up |
| Growth Factor (HB-EGF) | | | | | |
| Apolipoprotein A-I | 1.313 | 0.393 | 0.022 | 0.602 | up |
| (Apo A-I) | | | | | |
| Periostin | −1.473 | −0.559 | 0.023 | 0.602 | down |
| Alpha-Fetoprotein (AFP) | −1.746 | −0.804 | 0.025 | 0.602 | down |
| Angiopoietin-2 (ANG-2) | 1.408 | 0.493 | 0.026 | 0.602 | up |
| Cystatin-C | 1.339 | 0.421 | 0.029 | 0.602 | up |
| CD 40 antigen (CD40) | 1.450 | 0.536 | 0.030 | 0.602 | up |
| Hepsin | 1.458 | 0.544 | 0.034 | 0.620 | up |
| Apolipoprotein C-I | 1.266 | 0.340 | 0.035 | 0.620 | up |
| (Apo C-I) | | | | | |
| Interleukin-1 receptor | 1.302 | 0.381 | 0.042 | 0.629 | up |
| type 1 (IL-1RI) | | | | | |
| Eotaxin-2 | −1.542 | −0.625 | 0.043 | 0.629 | down |
| Myeloperoxidase (MPO) | −1.646 | −0.719 | 0.046 | 0.629 | down |
| Ferritin (FRTN) | −2.186 | −1.129 | 0.046 | 0.629 | down |

The analysis identified 13 proteins in the all patient data set as significantly different (p<0.05) between responders and non-responders in the pre-treatment samples (log FC=Log 2 fold change of the protein; a positive value indicates up regulation, while a negative value indicates down regulation in the comparison of interest):

| | | | P. | adj. | |
|---|---|---|---|---|---|
| Biomarker ID | FC | logFC | Value | P. Val | |
| Heparin-Binding EGF-Like | 1.417 | 0.503 | 0.002 | 0.426 | up |
| Growth Factor (HB-EGF) | | | | | |
| Alpha-Fetoprotein (AFP) | −1.642 | −0.715 | 0.005 | 0.426 | down |
| Human Chorionic | −1.224 | −0.292 | 0.005 | 0.426 | down |
| Gonadotropin beta (hCG) | | | | | |
| Interleukin-2 receptor alpha | 1.608 | 0.685 | 0.015 | 0.801 | up |
| (IL-2 receptor alpha) | | | | | |
| Latency-Associated | 1.545 | 0.628 | 0.017 | 0.801 | up |
| Peptide of Transforming | | | | | |
| Growth Factor beta 1 | | | | | |
| (LAP TGF-b1) | | | | | |
| Thymus and activation- | 2.104 | 1.073 | 0.020 | 0.801 | up |
| regulated chemokine | | | | | |
| (TARC) | | | | | |
| Neuronal Cell Adhesion | 1.717 | 0.780 | 0.024 | 0.801 | up |
| Molecule (Nr-CAM) | | | | | |
| CD40 Ligand (CD40-L) | 1.649 | 0.721 | 0.026 | 0.801 | up |
| Kidney Injury Molecule-1 | 1.346 | 0.428 | 0.029 | 0.801 | up |
| (KIM-1) | | | | | |
| CD 40 antigen (CD40) | 1.366 | 0.450 | 0.031 | 0.801 | up |
| Chromogranin-A (CgA) | 1.627 | 0.702 | 0.035 | 0.826 | up |
| Epidermal Growth Factor | 1.731 | 0.791 | 0.041 | 0.902 | up |
| (EGF) | | | | | |
| Vascular endothelial | 1.305 | 0.384 | 0.047 | 0.938 | up |
| growth factor receptor 3 | | | | | |
| (VEGFR-3) | | | | | |

### Example 10

This example describes the results from further analysis of trial BGBC008 (NCT03184571), which consists of samples from patients diagnosed with NSCLC. Subjects were administered the Axl inhibitor BGB324 in combination with pembrolizumab.

21 proteins were identified as significantly different (p<0.05) between responders and non-responders in the pre-treatment samples (log FC=Log 2 fold change of the protein; a positive value indicates up regulation, while a negative value indicates down regulation in the comparison of interest):

| | | | P. | adj. | |
|---|---|---|---|---|---|
| Biomarker ID | FC | logFC | Value | P. Val | |
| N-terminal | −5.742 | −2.521 | 2.01E−04 | 0.0529 | down |
| prohormone of | | | | | |
| brain natriuretic | | | | | |
| peptide (NT | | | | | |
| proBNP) | | | | | |
| Endostatin | −1.942 | −0.960 | 7.83E−04 | 0.1029 | down |
| Insulin-like Growth | −1.9352 | −0.953 | 0.006 | 0.490 | down |
| Factor-Binding | | | | | |
| Protein 2 (IGFBP-2) | | | | | |
| Vascular endothelial | −1.516 | −0.600 | 0.008 | 0.490 | down |
| growth factor D | | | | | |
| (VEGF-D) | | | | | |
| Growth/differentiation | −1.656 | −0.728 | 0.012 | 0.490 | down |
| factor 15 (GDF-15) | | | | | |
| Beta Amyloid | −1.879 | −0.910 | 0.014 | 0.490 | down |
| 1-40 (AB-40) | | | | | |
| Antileukoproteinase | −1.511 | −0.595 | 0.017 | 0.490 | down |
| (ALP) | | | | | |
| Monocyte Chemotactic | 1.694 | 0.760 | 0.019 | 0.490 | Up |
| Protein 3 (MCP-3) | | | | | |
| Superoxide Dismutase | 1.702 | 0.767 | 0.020 | 0.490 | Up |
| 1, soluble (SOD-1) | | | | | |
| Bone morphogenetic | 1.894 | 0.921 | 0.021 | 0.490 | Up |
| protein 9 (BMP-9) | | | | | |
| Interleukin-8 | 5.086 | 2.346 | 0.022 | 0.490 | Up |
| (IL-8) | | | | | |
| Macrophage | 1.984 | 0.988 | 0.022 | 0.490 | Up |
| Inflammatory | | | | | |
| Protein-1 beta | | | | | |
| (MIP-1 beta) | | | | | |
| Receptor for advanced | −1.930 | −0.949 | 0.027 | 0.549 | Down |
| glycosylation end | | | | | |
| products (RAGE) | | | | | |
| Myeloperoxidase | 2.163 | 1.113 | 0.031 | 0.578 | Up |
| (MPO) | | | | | |
| Chromogranin-A | −1.920 | −0.941 | 0.037 | 0.594 | Down |
| (CgA) | | | | | |
| HE4 | −1.649 | −0.722 | 0.041 | 0.594 | Down |
| Adrenomedullin | −1.668 | −0.738 | 0.041 | 0.594 | Down |
| (ADM) | | | | | |
| Beta-2- | −1.311 | −0.390 | 0.043 | 0.594 | Down |
| Microglobulin | | | | | |
| (B2M) | | | | | |
| Vitamin D-Binding | 1.275 | 0.351 | 0.044 | 0.594 | Down |
| Protein (VDBP) | | | | | |
| Cystatin-C | −1.248 | −0.320 | 0.045 | 0.594 | Down |
| Hepsin | −1.208 | −0.273 | 0.050 | 0.613 | Down |

### REFERENCES

A number of publications are cited above in order to more fully describe and disclose the disclosure and the state of the art to which the disclosure pertains. Full citations for these references are provided below. The entirety of each of these references is incorporated herein.
De Kouchkovsky et al, 2016, Blood Cancer J., 6(7):e441
Ben-Batalla et al, 2013, Blood, 122(14):2443-52
Burnett et al., 2011, J Clin Oncol. 29(5):487-94
Haferlach, 2008, Hematology Am Soc Hematol Educ Program, 1: 400-11
Ma et al, 2018, Medicine (Baltimore), 97(16):e0434
Hsueh et al, 2016, Exp Hematol Oncol., 5:23
Jhan et al, 2017, Pharmacogenomics, 18(17):1595-1609.
Herbst et al, 2018, Nature, 553(7689):446-454.
Zappa et al, 2016, Transl Lung Cancer Res. 5(3):288-300
WO2015/193428
WO2015/193430
WO2016/097370
WO2016/102672
WO2016/166296
For standard molecular biology techniques, see Sambrook, J., Russel, D. W. Molecular Cloning, A Laboratory Manual. 3 ed. 2001, Cold Spring Harbor, N.Y.: Cold Spring Harbor Laboratory Press

1. A method of predicting a cancer-related outcome in a subject, the method comprising assessing the activity, expression, or amount of one or more biomarker in the subject, or in a sample derived from the subject;
wherein the one or more biomarker is selected from the group consisting of: Axl, FGF-21, RAGE, CEA, FSH, MMP-10, Omentin, CA-19-9, LH, Haptoglobin, NAP-2, IgE, and/or CD40-L; and
wherein the subject has, is suspected of having, or has been diagnosed with acute myelocytic leukemia (AML).

2. A method according to claim 1, wherein the method comprises:
assessing the activity, expression, or amount of one or more biomarker in the subject, or in a sample derived from the subject, to obtain a sample profile of the one or more biomarker; and
making a prediction based on the sample profile of the one or more biomarker;
optionally wherein the prediction is made by comparing the sample profile to a control profile.

3. A method according to claim 2, wherein the sample profile is obtained before the subject is contacted with or administered an agent capable of inhibiting or reversing EMT or a chemotherapeutic agent.

4. A method according to claim 3, wherein the control profile is:
(i) obtained from a population of control subjects having AML; or
(ii) obtained from a control subject or population of control subjects having AML and previously found to lack susceptibility to treatment with an agent capable of inhibiting or reversing EMT or a chemotherapeutic agent;
(iii) a predetermined profile of “average, median, or mean” or “standard ranges” of biomarker expression, activity, or amount values obtained from a control subject or population of control subjects having AML and previously found to lack susceptibility to treatment with an agent capable of inhibiting or reversing EMT or a chemotherapeutic agent;
(iv) obtained from a control sample having a known “average, median, or mean” value of biomarkers indicative of a control subject or population of control subjects having AML and previously found to lack susceptibility to treatment with an agent capable of inhibiting or reversing EMT or a chemotherapeutic agent;
(v) a predetermined profile of biomarker expression, activity, or amount “threshold” values obtained from a control subject or population of control subjects having AML and previously found to be susceptible to treatment with an agent capable of inhibiting or reversing EMT or a chemotherapeutic agent; or
(vi) obtained from a control sample having a known “threshold” value of biomarkers indicative of a control subject or population of control subjects having AML and previously found to be susceptible to treatment with an agent capable of inhibiting or reversing EMT or a chemotherapeutic agent.

5. A method according to any one of claims 3-4, wherein the one or more biomarker includes at least one of: Haptoglobin, NAP-2, IgE, and/or CD40-L,
and wherein a higher activity, expression, or amount of one or more of said biomarkers in the sample profile as compared with the control profile is indicative of susceptibility to treatment with an agent capable of inhibiting or reversing EMT or a chemotherapeutic agent.

6. A method according to any one of claims 3-5, wherein the one or more biomarker includes at least one of: Axl, FGF-21, RAGE, CEA, FSH, MMP-10, Omentin, CA-19-9, and/or LH,
and wherein a lower activity, expression, or amount of one or more of said biomarkers in the sample profile as compared with the control profile is indicative of susceptibility to treatment with an agent capable of inhibiting or reversing EMT or a chemotherapeutic agent.

7. A method according to any one of claims 1-6, wherein the one or more biomarker comprises Axl,
optionally wherein the one or more biomarker comprises Axl and at least 1, 2, 3, 4, 5, 6, 7, 8, 9, 10, or more further biomarkers selected from: FGF-21, RAGE, CEA, FSH, MMP-10, Omentin, CA-19-9, LH, Haptoglobin, NAP-2, IgE, and/or CD40-L.

8. A method according to claim 2, wherein the sample profile is obtained after contacting the subject with an agent capable of inhibiting or reversing EMT or a chemotherapeutic agent,
optionally wherein the control profile is obtained from the same subject prior to contacting said subject with the agent capable of inhibiting or reversing EMT or a chemotherapeutic agent.

9. A method according to claim 8, wherein an increase in the activity, expression, or amount of one or more biomarker in the sample profile as compared with the control profile is indicative of higher susceptibility to treatment with an agent capable of inhibiting or reversing EMT or a chemotherapeutic agent.

10. A method according to claim 9, wherein the one or more biomarker includes at least Axl.

11. A method according to any one of claims 1-10, wherein the method comprises determining the amount of at least 2, 3, 4, 5, 6, 7, 8, 9, 10, 11, 12, 13, 14, 15, 20 of said biomarkers in the subject, or sample derived from the subject.

12. A method according to any one of claims 1-11, wherein assessing the expression or amount of one or more biomarker in the subject, or in a sample derived from the subject, comprises:
contacting a sample, or an extract from the sample, with at least 1, 2, 3, 4, 5, 6, 7, 8, 9, 10 or more specific binding members, each of which selectively binds to a respective biomarker protein; and
detecting and/or quantifying formation of complex formed by said specific binding member and said biomarker protein,
optionally wherein the specific binding member comprises an antibody molecule or binding fragment thereof.

13. A method according to any one of claims 3-12, wherein the agent capable of inhibiting or reversing EMT or chemotherapeutic agent is an Axl inhibitor.

14. A method according to claim 13, wherein the Axl inhibitor is BGB324/R428/bemcentinib.

15. A method according to claim 13, wherein the Axl inhibitor is an anti-Axl antibody described in WO2015/193428, WO2015/193430, WO2016/097370, or WO2016/166296

16. A method according to any one of claims 3-15, wherein the agent capable of inhibiting or reversing EMT is administered in combination with a further cancer treatment.

17. A method according to claim 16, wherein the further cancer treatment is cytarabine or decitabine.

18. A method according to any one of claims 1-17, wherein the subject is mammalian;
optionally wherein the subject is human.

19. A method according to any one of claims 1-18, wherein the sample is a blood, serum, or plasma sample;
optionally wherein the sample is a serum sample.

20. A method according to any one of any one of claims 1-19, wherein the method is performed in vitro or ex vivo.

21. A method of selecting subjects having, suspected of having, or diagnosed with acute myelocytic leukemia (AML) for treatment with an agent capable of inhibiting or reversing EMT or a chemotherapeutic agent, the method comprising:
identifying subjects susceptible to treatment with an agent capable of inhibiting or reversing EMT or a chemotherapeutic agent using a method according to any one of claims 1-20; and
selecting thus identified subjects for treatment.

22. A method of selecting subjects having, suspected of having, or diagnosed with acute myelocytic leukemia (AML) for continued treatment with an agent capable of inhibiting or reversing EMT or a chemotherapeutic agent, the method comprising:
identifying subjects susceptible to treatment with an agent capable of inhibiting or reversing EMT or a chemotherapeutic agent using a method according to any one of claims 8-20; and
selecting thus identified subjects for continued treatment.

23. A diagnostic kit or test device comprising:
1, 2, 3, 4, 5, 6, 7, 8, 9, 10 or more specific binding members, each of which selectively binds to a biomarker selected from the group consisting of: Axl, FGF-21, RAGE, CEA, FSH, MMP-10, Omentin, CA-19-9, LH, Haptoglobin, NAP-2, IgE, and/or CD40-L; and
one or more reagents for detecting said one or more specific binding members or one or more reagents for detecting and/or quantifying formation of a complex formed by said specific binding member and said biomarker.

24. A diagnostic kit or test device according to claim 23 for use in a method of predicting a cancer-related outcome in a subject,
optionally wherein the method is a method according to any one of claims 1-20.

25. Use of one or more of: Axl, FGF-21, RAGE, CEA, FSH, MMP-10, Omentin, CA-19-9, LH, Haptoglobin, NAP-2, IgE, and/or CD40-L, as a biomarker for determining if a subject is susceptible to treatment with an agent capable of inhibiting or reversing EMT or a chemotherapeutic agent,
optionally wherein the use is as a biomarker in a method according to any one of claims 1-20.
